(19) United States
(12) Patent Application Publication
(10) Pub. No.: US 2023/0382910 A1
(43) Pub. Date: Nov. 30, 2023

(54) HETEROARYL COMPOUNDS FOR THE TREATMENT OF PAIN

(71) Applicant: VERTEX PHARMACEUTICALS INCORPORATED, Boston, MA (US)

(72) Inventors: Mark Thomas Miller, Rancho Santa Fe, CA (US); Dennis James Hurley, San Marcos, CA (US); Timothy Donald Neubert, San Diego, CA (US); Elamprakash Nesan Savariar, San Diego, CA (US); Urvi Patel, San Diego, CA (US); Sara Sabina Hadida Ruah, La Jolla, CA (US); Jason McCartney, Cardiff by the Sea, CA (US); Jinglan Zhou, San Diego, CA (US); Robert Martin Demoret, San Diego, CA (US); Roman Askatovich Valiulin, San Diego, CA (US); Alexander Frederik Kintzer, Carlsbad, CA (US); Peter Webb, San Diego, CA (US); David Robert Slochower, San Diego, CA (US); Kathleen Aertgeerts, San Diego, CA (US); Elizabeth Mary Beck, Abingdon (GB); Ronald Marcellus Alphonsus Knegtel, Abingdon, (GB); Ewa Iwona Chudyk, Wantage (GB); Joanne Louise Pinder, Didcot (GB)

(21) Appl. No.: 18/305,186

(22) Filed: Apr. 21, 2023

Related U.S. Application Data
(60) Provisional application No. 63/333,873, filed on Apr. 22, 2022.

(51) Int. Cl. C07D 471/04; C07D 215/233; C07D 215/22; C07D 215/60; C07D 215/48; C07D 401/04

(52) U.S. Cl. CPC C07D 471/04; C07D 215/233; C07D 215/22; C07D 215/60; C07D 215/48; C07D 401/04

(57) ABSTRACT

Compounds, and pharmaceutically acceptable salts thereof, useful as inhibitors of sodium channels are provided. Also provided are pharmaceutical compositions comprising the compounds or pharmaceutically acceptable salts and methods of using the compounds, pharmaceutically acceptable salts, and pharmaceutical compositions in the treatment of various disorders, including pain.

### CROSS-REFERENCE TO RELATED APPLICATIONS

This application claims the benefit of U.S. Provisional Application No. 63/333,873, filed Apr. 22, 2022, which is incorporated by reference herein in its entirety.

### BACKGROUND

Pain is a protective mechanism that allows healthy animals to avoid tissue damage and to prevent further damage to injured tissue. Nonetheless, there are many conditions where pain persists beyond its usefulness, or where patients would benefit from inhibition of pain. Neuropathic pain is a form of chronic pain caused by an injury to the sensory nerves (Dieleman, J. P., et al., Incidence rates and treatment of neuropathic pain conditions in the general population. Pain, 2008. 137(3): p. 681-8). Neuropathic pain can be divided into two categories, pain caused by generalized metabolic damage to the nerve and pain caused by a discrete nerve injury. The metabolic neuropathies include post-herpetic neuropathy, diabetic neuropathy, and drug-induced neuropathy. Discrete nerve injury indications include post-amputation pain, post-surgical nerve injury pain, and nerve entrapment injuries like neuropathic back pain.

Voltage-gated sodium channels (Na_{V}s) are involved in pain signaling. Na_{V}s are biological mediators of electrical signaling as they mediate the rapid upstroke of the action potential of many excitable cell types (e.g. neurons, skeletal myocytes, cardiac myocytes). The evidence for the role of these channels in normal physiology, the pathological states arising from mutations in sodium channel genes, preclinical work in animal models, and the clinical pharmacology of known sodium channel modulating agents all point to the central role of Na_{V}s in pain sensation (Rush, A. M. and T. R. Cummins, Painful Research: Identification of a Small-Molecule Inhibitor that Selectively Targets Na_{V}1.8 Sodium Channels. Mol. Interv., 2007. 7(4): p. 192-5); England, S., Voltage-gated sodium channels: the search for subtype-selective analgesics. Expert Opin. Investig. Drugs 17 (12), p. 1849-64 (2008); Krafte, D. S. and Bannon, A. W., Sodium channels and nociception: recent concepts and therapeutic opportunities. Curr. Opin. Pharmacol. 8 (1), p. 50-56 (2008)). Na_{V}s mediate the rapid upstroke of the action potential of many excitable cell types (e.g. neurons, skeletal myocytes, cardiac myocytes), and thus are involved in the initiation of signaling in those cells (Hille, Bertil, Ion Channels of Excitable Membranes, Third ed. (Sinauer Associates, Inc., Sunderland, M A, 2001)). Because of the role Na_{V}s play in the initiation and propagation of neuronal signals, antagonists that reduce Na_{V} currents can prevent or reduce neural signaling and Na_{V} channels have been considered likely targets to reduce pain in conditions where hyper-excitability is observed (Chahine, M., Chatelier, A., Babich, O., and Krupp, J. J., Voltage-gated sodium channels in neurological disorders. CNS Neurol. Disord. Drug Targets 7 (2), p. 144-58 (2008)). Several clinically useful analgesics have been identified as inhibitors of Na_{V} channels. The local anesthetic drugs such as lidocaine block pain by inhibiting Na_{V} channels, and other compounds, such as carbamazepine, lamotrigine, and tricyclic antidepressants that have proven effective at reducing pain have also been suggested to act by sodium channel inhibition (Soderpalm, B., Anticonvulsants: aspects of their mechanisms of action. Eur. J. Pain 6 Suppl. A, p. 3-9 (2002); Wang, G. K., Mitchell, J., and Wang, S. Y., Block of persistent late Na⁺ currents by antidepressant sertraline and paroxetine. J. Membr. Biol. 222 (2), p. 79-90 (2008)).

The Na_{V}s form a subfamily of the voltage-gated ion channel super-family and comprises 9 isoforms, designated Na_{V}1.1-Na_{V}1.9. The tissue localizations of the nine isoforms vary. Na_{V}1.4 is the primary sodium channel of skeletal muscle, and Na_{V}1.5 is primary sodium channel of cardiac myocytes. Na_{V}s 1.7, 1.8 and 1.9 are primarily localized to the peripheral nervous system, while Na_{V}s 1.1, 1.2, 1.3, and 1.6 are neuronal channels found in both the central and peripheral nervous systems. The functional behaviors of the nine isoforms are similar but distinct in the specifics of their voltage-dependent and kinetic behavior (Catterall, W. A., Goldin, A. L., and Waxman, S. G., International Union of Pharmacology. XLVII. Nomenclature and structure-function relationships of voltage-gated sodium channels. Pharmacol. Rev. 57 (4), p. 397 (2005)).

Upon their discovery, Na_{V}1.8 channels were identified as likely targets for analgesia (Akopian, A. N., L. Sivilotti, and J. N. Wood, A tetrodotoxin-resistant voltage-gated sodium channel expressed by sensory neurons. Nature, 1996. 379(6562): p. 257-62). Since then, Na_{V}1.8 has been shown to be a carrier of the sodium current that maintains action potential firing in small dorsal root ganglia (DRG) neurons (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22(23): p. 10277-90). Na_{V}1.8 is involved in spontaneous firing in damaged neurons, like those that drive neuropathic pain (Roza, C., et al., The tetrodotoxin-resistant Na⁺ channel Na_{V}1.8 is essential for the expression of spontaneous activity in damaged sensory axons of mice. J. Physiol., 2003. 550(Pt 3): p. 921-6; Jarvis, M. F., et al., A-803467, a potent and selective Na_{V}1.8 sodium channel blocker, attenuates neuropathic and inflammatory pain in the rat. Proc. Natl. Acad. Sci. USA, 2007. 104(20): p. 8520-5; Joshi, S. K., et al., Involvement of the TTX-resistant sodium channel Na_{V}1.8 in inflammatory and neuropathic, but not post-operative, pain states. Pain, 2006. 123(1-2): pp. 75-82; Lai, J., et al., Inhibition of neuropathic pain by decreased expression of the tetrodotoxin-resistant sodium channel, Na_{V}1.8. Pain, 2002. 95(1-2): p. 143-52; Dong, X. W., et al., Small interfering RNA-mediated selective knockdown of Na_{V}1.8 tetrodotoxin-resistant sodium channel reverses mechanical allodynia in neuropathic rats. Neuroscience, 2007. 146(2): p. 812-21; Huang, H. L., et al., Proteomic profiling of neuromas reveals alterations in protein composition and local protein synthesis in hyper-excitable nerves. Mol. Pain, 2008. 4: p. 33; Black, J. A., et al., Multiple sodium channel isoforms and mitogen-activated protein kinases are present in painful human neuromas. Ann. Neurol., 2008. 64(6): p. 644-53; Coward, K., et al., Immunolocalization of SNS/PN3 and NaN/SNS2 sodium channels in human pain states. Pain, 2000. 85(1-2): p. 41-50; Yiangou, Y., et al., SNS/PN3 and SNS2/NaN sodium channel-like immunoreactivity in human adult and neonate injured sensory nerves. FEBS Lett., 2000. 467(2-3): p. 249-52; Ruangsri, S., et al., Relationship of axonal voltage-gated sodium channel 1.8 (Na_{V}1.8) mRNA accumulation to sciatic nerve injury-induced painful neuropathy in rats. J. Biol. Chem. 286(46): p. 39836-47). The small DRG neurons where Na_{V}1.8 is expressed include the nociceptors involved in pain signaling. Na_{V}1.8 mediates large amplitude action potentials in small neurons of the dorsal root ganglia (Blair, N. T. and B. P. Bean, Roles of tetrodotoxin (TTX)-sensitive Na⁺ current, TTX-resistant Na⁺ current, and Ca²⁺ current in the action potentials of nociceptive sensory neurons. J. Neurosci., 2002. 22(23): p. 10277-90). Na_{V}1.8 is necessary for rapid repetitive action potentials in nociceptors, and for spontaneous activity of damaged neurons. (Choi, J. S. and S. G. Waxman, Physiological interactions between Na_{V}1.7 and Na_{V}1.8 sodium channels: a computer simulation study. J. Neurophysiol. 106(6): p. 3173-84; Renganathan, M., T. R. Cummins, and S. G. Waxman, Contribution of Na(_{V})1.8 sodium channels to action potential electrogenesis in DRG neurons. J. Neurophysiol., 2001. 86(2): p. 629-40; Roza, C., et al., The tetrodotoxin-resistant Na⁺ channel Na_{V}1.8 is essential for the expression of spontaneous activity in damaged sensory axons of mice. J. Physiol., 2003. 550(Pt 3): p. 921-6). In depolarized or damaged DRG neurons, Na_{V}1.8 appears to be a driver of hyper-excitability (Rush, A. M., et al., A single sodium channel mutation produces hyper- or hypoexcitability in different types of neurons. Proc. Natl. Acad. Sci. USA, 2006. 103(21): p. 8245-50). In some animal pain models, Na_{V}1.8 mRNA expression levels have been shown to increase in the DRG (Sun, W., et al., Reduced conduction failure of the main axon of polymodal nociceptive C-fibers contributes to painful diabetic neuropathy in rats. Brain, 135(Pt 2): p. 359-75; Strickland, I. T., et al., Changes in the expression of Na_{V}1.7, Na_{V}1.8 and Na_{V}1.9 in a distinct population of dorsal root ganglia innervating the rat knee joint in a model of chronic inflammatory joint pain. Eur. J Pain, 2008. 12(5): p. 564-72; Qiu, F., et al., Increased expression of tetrodotoxin-resistant sodium channels Na_{V}1.8 and Na_{V}1.9 within dorsal root ganglia in a rat model of bone cancer pain. Neurosci. Lett., 512(2): p. 61-6).

The inventors have discovered that some voltage-gated sodium channel inhibitors have limitations as therapeutic agents due to, for example, a poor therapeutic window (e.g., due to a lack of Na_{V} isoform selectivity, low potency, and/or other reasons). Accordingly, there remains a need to develop selective voltage-gated sodium channel inhibitors, such as selective Na_{V}1.8 inhibitors.

### SUMMARY

In one aspect, the invention relates to a compound described herein, or a pharmaceutically acceptable salt thereof.

In another aspect, the invention relates to a pharmaceutical composition comprising the compound, or a pharmaceutically acceptable salt thereof, and one or more pharmaceutically acceptable carriers or vehicles.

In still another aspect, the invention relates to a method of inhibiting a voltage gated sodium channel in a subject by administering the compound, pharmaceutically acceptable salt, or pharmaceutical composition to the subject.

In yet another aspect, the invention relates to a method of treating or lessening the severity in a subject of a variety of diseases, disorders, or conditions, including, but not limited to, chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, and cardiac arrhythmia, by administering the compound, pharmaceutically acceptable salt, or pharmaceutical composition to the subject.

### DETAILED DESCRIPTION

In one aspect, the invention relates to a compound of formula (I):

or a pharmaceutically acceptable salt thereof, wherein:
X₅ₐ is N, N⁺—O⁻, or N⁺—CH₃;
X₉ₐ is N or CR^{9a};
R^{3a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
R^{4a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
R^{6a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —NHC(O)NH₂, —NHC(O)NH(C₁-C₆ alkyl), —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′};
R^{9a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
each R^{a′} is independently halo, —OH, C₁-C₆ alkyl, C₁-C₆ haloalkyl, —C(O)O(C₁-C₆ alkyl), or —C(O)NH₂;
Y is

X_{2b} is N or CR^{2b};
X_{3b} is N or CR^{3b};
X_{4b} is N or CR^{4b};
X_{5b} is N or CR^{5b};
X_{6b} is N or CR^{6b};
Z is a 5-7 membered aromatic or nonaromatic ring optionally containing 1-3 heteroatoms selected from nitrogen and oxygen and optionally substituted with one or more R^{z};
R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{3b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ haloalkylene)-OH, —(C₁-C₆ alkylene)-C(O)OH, —(C₁-C₆ haloalkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′};
R^{5b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{6b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl,
   (C₁-C₆ alkyl)-(C₃-C₆ cycloalkyl)-, or (C₁-C₆ haloalkyl)-(C₃-C₆ cycloalkyl)- is optionally substituted with one or more halo;
R^{7b} is C₁-C₆ alkyl;
R^{9b} is C₁-C₆ alkyl;
R^{10b} is H or halo;
each R^{b′} is independently C₁-C₆ alkyl or C₁-C₆ haloalkyl; and
R^{z} is halo, C₁-C₆ alkyl, C₁-C₆ alkenyl, C₁-C₆ alkoxy, C₁-C₆ haloalkyl, C₁-C₆ haloalkenyl, —CN, —C(O)NH₂, —C(O)NH(C₁-C₆ alkyl), —C(O)N(C₁-C₆ alkyl)₂, or —C(O)OH;
   provided that:
(i) if Y is

X_{3b} is CR^{3b}, and X_{6b}, is CR^{6b}, then no more than three of R^{2b}, R^{3b}, R^{4b}, R^{5b}, and R^{6b} are H; and
(ii) if Y is

X_{3b} is N, and X_{6b} is CR^{6b}, then no more than three of R^{2b}, R^{4b}, R^{5b}, and R^{6b} are H; and
(iii) if Y is

X_{3b} is CR^{3b}, and X_{6b} is N, then no more than three of R^{2b}, R^{3b}, R^{4b}, and R^{5b} are H; and
(iv) if X₉ₐ is

X_{3b} is CR^{3b}, and X_{6b} is CR^{6b}, then no more than two of R^{2b}, R^{3b}, R^{4b}, R^{5b}, and R^{6b} are halo; and
(v) if X₉ₐ is

X_{4b} is CR^{4b}, X_{5b} is CR^{5b}, and X_{6b} is CR^{6b}, then:
   no more than two of R^{4b}, R^{5b}, and R^{6b} are H; or
   Z is substituted with one or more R^{z}.

In another aspect, the invention relates to a compound of formula (II):

or a pharmaceutically acceptable salt thereof, wherein:
R^{3c} is H, halo, or C₁-C₆ alkyl;
R^{4c} is H, halo, —CN, or C₁-C₆ alkoxy;
R^{5c} is H, halo, —CN, C₁-C₆ alkyl, C₁-C₆ haloalkyl, C₁-C₆ alkoxy, or —C(O)O(C₁-C₆ alkyl);
R^{6c} is H, halo, —OH, —CN, C₁-C₆ alkoxy, C(O)NH₂, or 5-10 membered heteroaryl comprising 1-3 heteroatoms selected from nitrogen and oxygen;
R^{9c} is H, C₁-C₆ alkyl, —(C₁-C₆ alkylene)-OH, —(C₁-C₆ alkylene)-O(C₁-C₆ alkyl), or —C(O)O(C₁-C₆ alkyl);
Y is

X_{3d} is N or CR^{3d};
   R^{2d}, R^{3d}, and R^{4d} are defined as follows:
(i) R^{2d} is H, halo, —OH, C₁-C₆ alkyl, C₁-C₆ alkoxy, C₁-C₆ haloalkoxy, or —(C₁-C₆ alkylene)-OH;
   R^{3d} is H, halo, C₁-C₆ alkyl, C₁-C₆ haloalkyl, C₁-C₆ alkoxy, or C₁-C₆ haloalkoxy; and
   R^{4d} is H, halo, C₁-C₆ alkyl, C₁-C₆ haloalkyl, C₁-C₆ haloalkenyl, C₁-C₆ alkoxy, C₁-C₆ haloalkoxy, —(C₁-C₆ alkylene)-OH, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN; or (ii) R^{2d} and R^{3d}, together with the carbon atoms to which they are attached, form a ring of formula:

and
   R^{4d} is H, halo, C₁-C₆ alkyl, C₁-C₆ haloalkyl, C₁-C₆ haloalkenyl, C₁-C₆ alkoxy, C₁-C₆ haloalkoxy, —(C₁-C₆ alkylene)-OH, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN; or
(iii) R^{2d} is H, halo, —OH, C₁-C₆ alkyl, C₁-C₆ alkoxy, C₁-C₆ haloalkoxy, or —(C₁-C₆ alkylene)-OH; and
   R^{3d} and R^{4d}, together with the carbon atoms to which they are attached, form a ring of formula:

R^{5d} is H, halo, C₁-C₆ alkyl, C₁-C₆ haloalkyl, C₁-C₆ alkoxy, or —C(O)(C₁-C₆ alkyl);
R^{6d} is H, halo, or C₁-C₆ alkyl;
R^{7d} is C₁-C₆ alkyl; and
R^{9d} is C₁-C₆ alkyl;
   provided that:
(i) if Y′ is

and X_{3d} is CR^{3d}, then no more than four of R^{2d}, R^{3d}, R^{4d}, R^{5d}, and R^{6d} are H; and
(ii) if Y′ is

and X_{3d} is N, then no more than three of R^{2d}, R^{4d}, R^{5d}, and R^{6d} are H.

In another aspect, the invention relates to a compound of formula (III):

or a pharmaceutically acceptable salt thereof, wherein:
X₃ₖ is N or CH;
X₄ₖ is N or CH;
X₅ₖ is N or CR^{5k};
X₆ₖ is N, N⁺—O⁻, or CR^{6k};
R^{5k} is H, C₁-C₆ alkoxy, —OH, —OCH₂CH₂N(CH₃)₂, or —N(CH₃)(CH₂CH₂OCH₃);
R^{6k} is H, —OH, C₁-C₆ alkoxy, or —C(O)NH₂;
R^{2L} is C₁-C₆ alkyl;
R^{4L} is C₁-C₆ alkyl, C₁-C₆ haloalkyl, or (C₁-C₆ haloalkyl)-(C₃-C₆ cycloalkyl)-; and
R^{5L} is H, halo, or C₁-C₆ alkyl,
   provided that:
(i) at least one of X₃ₖ, X₄ₖ, and X₅ₖ is N, or X₆ₖ is N or N⁺—O⁻; and
(ii) no more than two of X₃ₖ, X₄ₖ, X₅ₖ, and X₆ₖ are N; and
(iii) if X₆ₖ is N⁺—O⁻, then X₃ₖ and X₄ₖ are CH, and X₅ₖ is CR^{5k}; and
(iv) if X₅ₖ is N, then X₄ₖ is N.

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry,” Thomas Sorrell, University Science Books, Sausalito: 1999, and “March's Advanced Organic Chemistry,” 5^{th} Ed., Ed.: Smith, M. B. and March, J., John Wiley & Sons, New York: 2001, the entire contents of which are hereby incorporated by reference.

As used herein, the term “compounds of the invention” refers to the compounds of formulas (I), (II), and (III) and all of the embodiments thereof (e.g., formulas (I-A), (I-B), (I-C), (I-D), (I-E), (I-F), (I-G), (II-A), (II-B), (II-C), (III-A), etc.), as described herein, and to the compounds identified in Table A and Table B.

As described herein, the compounds of the invention comprise multiple variable groups (e.g., X₅ₐ, R^{4a}, Y, Z, etc.). As one of ordinary skill in the art will recognize, combinations of groups envisioned by this invention are those combinations that result in the formation of stable or chemically feasible compounds. The term “stable,” in this context, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The chemical structures depicted herein are intended to be understood as they would be understood by one of ordinary skill in the art. For example, with respect to formulas (III) and (III-A), X₆ₖ and X₅ₖ are connected by a double bond, and X₅ₖ and X₄ₖ are connected by a single bond, even though the bonds between these groups may be obscured by the atom labels in the chemical structures. Moreover, a substituent depicted as “CF₃” or “F₃C” in a chemical structure refers to a trifluoromethyl substituent, regardless of which depiction appears in the chemical structure.

As used herein, the term “halo” means F, Cl, Br or I.

As used herein, the term “alkyl” refers to a straight or branched hydrocarbon chain radical group consisting solely of carbon and hydrogen atoms, containing no unsaturation, and having the specified number of carbon atoms, which is attached to the rest of the molecule by a single bond. For example, a “C₁-C₆ alkyl” group is an alkyl group having between one and six carbon atoms.

As used herein, the term “alkenyl” refers to a straight or branched hydrocarbon chain radical group consisting solely of carbon and hydrogen atoms, containing one or more carbon-carbon double bonds, and having the specified number of carbon atoms, which is attached to the rest of the molecule by a single bond. For example, a “C₂-C₆ alkenyl” group is an alkenyl group having between two and six carbon atoms.

As used herein, the term “cycloalkyl” refers to a stable, non-aromatic, mono- or bicyclic (fused, bridged, or spiro) saturated hydrocarbon radical consisting solely of carbon and hydrogen atoms, having the specified number of carbon ring atoms, and which is attached to the rest of the molecule by a single bond. For example, a “C₃-C₈ cycloalkyl” group is a cycloalkyl group having between three and eight carbon atoms.

As used herein, the term “alkoxy” refers to a radical of the formula —ORₐ where Rₐ is an alkyl group having the specified number of carbon atoms. For example, a “C₁-C₆ alkoxy” group is a radical of the formula —ORₐ where Rₐ is an alkyl group having the between one and six carbon atoms.

As used herein, the term “haloalkyl” refers to an alkyl group having the specified number of carbon atoms, wherein one or more of the hydrogen atoms of the alkyl group are replaced by halo groups. For example, a “C₁-C₆ haloalkyl” group is an alkyl group having between one and six carbon atoms, wherein one or more of the hydrogen atoms of the alkyl group are replaced by halo groups.

As used herein, the term “haloalkenyl” refers to an alkenyl group having the specified number of carbon atoms, wherein one or more of the hydrogen atoms of the alkenyl group are replaced by halo groups. For example, a “C₁-C₆ haloalkenyl” group is an alkenyl group having between one and six carbon atoms, wherein one or more of the hydrogen atoms of the alkenyl group are replaced by halo groups.

As used herein, the term “haloalkoxy” refers to an alkoxy group having the specified number of carbon atoms, wherein one or more of the hydrogen atoms of the of the alkyl group are replaced by halo groups.

As used herein, the term “alkylene” refers to a divalent, straight or branched hydrocarbon chain radical group consisting solely of carbon and hydrogen atoms, containing no unsaturation, and having the specified number of carbon atoms, which is attached to the rest of the molecule by two single bonds. For example, a “C₁-C₆ alkylene” group is an alkylene group having between one and six carbon atoms.

As used herein, the term “alkenylene” refers to a divalent, straight or branched hydrocarbon chain radical group consisting solely of carbon and hydrogen atoms, containing one or more carbon-carbon double bonds, and having the specified number of carbon atoms, which is attached to the rest of the molecule by two single bonds. For example, a “C₂-C₆ alkenylene” is an alkenylene group having between one and six carbon atoms.

As used herein, the term “haloalkylene” refers to an alkylene group having the specified number of carbon atoms, wherein one or more of the hydrogen atoms of the alkylene group are replaced by halo groups. For example, a “C₁-C₆ haloalkylene” group is an alkylene group having between one and six carbon atoms, wherein one or more of the hydrogen atoms of the alkylene group are replaced by halo groups.

As used herein, the term “heterocyclyl” refers to a stable, non-aromatic, mono-, bi-, or tricyclic (fused, bridged, or spiro) radical in which one or more ring atoms is a heteroatom (e.g., a heteroatom independently selected from N, O, P, and S), which has the specified number of ring atoms, and which is attached to the rest of the molecule by a single bond. Heterocyclic rings can be saturated, or can contain one or more double or triple bonds. In some embodiments, the “heterocyclyl” group has the indicated number of ring members, in which one or more ring members is a heteroatom independently selected from oxygen, sulfur, nitrogen, and phosphorus, and each ring in the ring system contains 3 to 7 ring members. For example, a 6-membered heterocyclyl includes a total of 6 ring members, at least one of which is a heteroatom (e.g., a heteroatom independently selected from N, O, P, and S).

As used herein, the term “heteroaryl” refers to a stable mono-, bi-, or tricyclic ring radical having the specified number of ring atoms, wherein at least one ring in the system is aromatic, at least one aromatic ring in the system contains one or more heteroatoms (e.g., one or more heteroatoms independently selected from N, O, P, and S). In some embodiments, each ring in the system contains 3 to 7 ring members. For example, a 6-membered heteroaryl includes a total of 6 ring members, at least one of which is a heteroatom selected from N, S, O, and P. The term “heteroaryl” may be used interchangeably with the term “heteroaryl ring” or the term “heteroaromatic”.

As used herein, labels such as “*2” and “*3”, such as those shown in the following structures, designate the carbon atoms to which the corresponding R groups (in this case, the R^{2d} and R^{3d} groups, respectively) are attached:

Similarly, “*3” and “*4” in the following structures designate the carbon atoms to which the R^{3d} and R^{4d} groups, respectively, are attached:

Unless otherwise specified, the compounds of the invention, whether identified by chemical name or chemical structure, include all stereoisomers (e.g., enantiomers and diastereomers), double bond isomers (e.g., (Z) and (E)), conformational isomers, and tautomers of the compounds identified by the chemical names and chemical structures provided herein. In addition, single stereoisomers, double bond isomers, conformational isomers, and tautomers as well as mixtures of stereoisomers, double bond isomers, conformational isomers, and tautomers are within the scope of the invention.

As used herein, in any chemical structure or formula, a non-bold, straight bond attached to a stereocenter of a compound, such as in

denotes that the configuration of the stereocenter is unspecified. The compound may have any configuration, or a mixture of configurations, at the stereocenter.

As used herein, the prefix “rac-,” when used in connection with a chiral compound, refers to a racemic mixture of the compound.

As used herein, the prefix “rel-,” when used in connection with a chiral compound, refers to a single enantiomer of unknown absolute configuration. In a compound bearing the “rel-” prefix, the (R)- and (S)-designators in the chemical name reflect the relative stereochemistry of the compound, but do not necessarily reflect the absolute stereochemistry of the compound. Where the relative stereochemistry of a given stereocenter is unknown, no stereochemical designator is provided.

As used herein, the term “compound,” when referring to the compounds of the invention, refers to a collection of molecules having identical chemical structures, except that there may be isotopic variation among the constituent atoms of the molecules. The term “compound” includes such a collection of molecules without regard to the purity of a given sample containing the collection of molecules. Thus, the term “compound” includes such a collection of molecules in pure form, in a mixture (e.g., solution, suspension, colloid, or pharmaceutical composition, or dosage form) with one or more other substances, or in the form of a hydrate, solvate, or co-crystal.

In the specification and claims, unless otherwise specified, any atom not specifically designated as a particular isotope in any compound of the invention is meant to represent any stable isotope of the specified element. In the Examples, where an atom is not specifically designated as a particular isotope in any compound of the invention, no effort was made to enrich that atom in a particular isotope, and therefore a person of ordinary skill in the art would understand that such atom likely was present at approximately the natural abundance isotopic composition of the specified element.

As used herein, the term “stable,” when referring to an isotope, means that the isotope is not known to undergo spontaneous radioactive decay. Stable isotopes include, but are not limited to, the isotopes for which no decay mode is identified in V. S. Shirley & C. M. Lederer, Isotopes Project, Nuclear Science Division, Lawrence Berkeley Laboratory, Table of Nuclides (January 1980).

As used herein in the specification and claims, “H” refers to hydrogen and includes any stable isotope of hydrogen, namely ¹H and D. In the Examples, where an atom is designated as “H,” no effort was made to enrich that atom in a particular isotope of hydrogen, and therefore a person of ordinary skill in the art would understand that such hydrogen atom likely was present at approximately the natural abundance isotopic composition of hydrogen.

As used herein, “¹H” refers to protium. Where an atom in a compound of the invention, or a pharmaceutically acceptable salt thereof, is designated as protium, protium is present at the specified position with at least the natural abundance concentration of protium.

As used herein, “D,” “d,” and “²H” refer to deuterium.

In some embodiments, the compounds of the invention, and pharmaceutically acceptable salts thereof, include each constituent atom at approximately the natural abundance isotopic composition of the specified element.

In some embodiments, the compounds of the invention, and pharmaceutically acceptable salts thereof, include one or more atoms having an atomic mass or mass number which differs from the atomic mass or mass number of the most abundant isotope of the specified element (“isotope-labeled” compounds and salts). Examples of stable isotopes which are commercially available and suitable for the invention include without limitation isotopes of hydrogen, carbon, nitrogen, oxygen, and phosphorus, for example ²H, ¹³C, ¹⁵N, ¹⁸O, ¹⁷O, and ³¹P, respectively.

The isotope-labeled compounds and salts can be used in a number of beneficial ways, including as medicaments. In some embodiments, the isotope-labeled compounds and salts are deuterium (²H)-labeled. Deuterium (²H)-labeled compounds and salts are therapeutically useful with potential therapeutic advantages over the non-²H-labeled compounds. In general, deuterium (²H)-labeled compounds and salts can have higher metabolic stability as compared to those that are not isotope-labeled owing to the kinetic isotope effect described below. Higher metabolic stability translates directly into an increased in vivo half-life or lower dosages, which under most circumstances would represent a preferred embodiment of the present invention. The isotope-labeled compounds and salts can usually be prepared by carrying out the procedures disclosed in the synthesis schemes, the Examples and the related description, replacing a non-isotope-labeled reactant by a readily available isotope-labeled reactant.

The deuterium (²H)-labeled compounds and salts can manipulate the rate of oxidative metabolism of the compound by way of the primary kinetic isotope effect. The primary kinetic isotope effect is a change of the rate for a chemical reaction that results from exchange of isotopic nuclei, which in turn is caused by the change in ground state energies of the covalent bonds involved in the reaction. Exchange of a heavier isotope usually results in a lowering of the ground state energy for a chemical bond and thus causes a reduction in the rate-limiting bond breakage. If the bond breakage occurs in or in the vicinity of a saddle-point region along the coordinate of a multi-product reaction, the product distribution ratios can be altered substantially. For example, if deuterium is bonded to a carbon atom at a non-exchangeable position, rate differences of k_{H}/k_{D}=2-7 are typical. For a further discussion, see S. L. Harbeson and R. D. Tung, Deuterium In Drug Discovery and Development, Ann. Rep. Med. Chem. 2011, 46, 403-417, incorporated in its entirety herein by reference.

The concentration of an isotope (e.g., deuterium) incorporated at a given position of an isotope-labeled compound of the invention, or a pharmaceutically acceptable salt thereof, may be defined by the isotopic enrichment factor. The term “isotopic enrichment factor,” as used herein, means the ratio between the abundance of an isotope at a given position in an isotope-labeled compound (or salt) and the natural abundance of the isotope.

Where an atom in a compound of the invention, or a pharmaceutically acceptable salt thereof, is designated as deuterium, such compound (or salt) has an isotopic enrichment factor for such atom of at least 3000 (˜45% deuterium incorporation). In some embodiments, the isotopic enrichment factor is at least 3500 (˜52.5% deuterium incorporation), at least 4000 (˜60% deuterium incorporation), at least 4500 (˜67.5% deuterium incorporation), at least 5000 (˜75% deuterium incorporation), at least 5500 (˜82.5% deuterium incorporation), at least 6000 (˜90% deuterium incorporation), at least 6333.3 (˜95% deuterium incorporation), at least 6466.7 (˜97% deuterium incorporation), at least 6600 (˜99% deuterium incorporation), or at least 6633.3 (˜99.5% deuterium incorporation).

In some embodiments, the invention relates to a compound of formula (I), wherein:
R^{6a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
Y is

R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo.

In some embodiments, the invention relates to a compound of formula (I), or a pharmaceutically acceptable salt thereof, wherein X₅ₐ is N. In some embodiments, the invention relates to a compound of formula (I), or a pharmaceutically acceptable salt thereof, wherein X₅ₐ is N⁺—O⁻. In some embodiments, the invention relates to a compound of formula (I), or a pharmaceutically acceptable salt thereof, wherein X₅ₐ is N⁺—CH₃.

In some embodiments, the invention relates to a compound of formula (I), or a pharmaceutically acceptable salt thereof, wherein X₉ₐ is CR^{9a}. In some embodiments, the invention relates to a compound of formula (I), wherein X₉ₐ is CR^{9a} and R^{9a} is H, halo, C₁-C₆ alkyl, C₁-C₆ alkoxy, —C(O)OH, or —C(O)O(C₁-C₆ alkyl). In some embodiments, the invention relates to a compound of formula (I), or a pharmaceutically acceptable salt thereof, wherein X₉ₐ is CR^{9a} and R^{9a} is H, halo, C₁-C₆ alkyl, or C₁-C₆ alkoxy. In other embodiments, R^{9a} is halo. In other embodiments, R^{9a} is C₁-C₆ alkyl. In other embodiments, R^{9a} is C₁-C₆ alkoxy. In other embodiments, R^{9a} is —C(O)OH. In other embodiments, R^{9a} is —C(O)O(C₁-C₆ alkyl). In some embodiments, the invention relates to a compound of formula (I), R^{9a} is H, Cl, Br, —CH₃, —OCH₃, —OCH₂CH₃, —C(O)OH, or —C(O)OCH₃. In some embodiments, the invention relates to a compound of formula (I), or a pharmaceutically acceptable salt thereof, wherein X₉ₐ is CR^{9a} and R^{9a} is H, Cl, Br, —CH₃, —OCH₃, or —OCH₂CH₃. In other embodiments, R^{9a} is H. In other embodiments, R^{9a} is Cl. In other embodiments, R^{9a} is Br. In other embodiments, R^{9a} is —CH₃. In other embodiments, R^{9a} is —OCH₃. In other embodiments, R^{9a} is —OCH₂CH₃. In other embodiments, R^{9a} is —C(O)OCH₃.

In some embodiments, the invention relates to a compound of formula (I), or a pharmaceutically acceptable salt thereof, wherein R^{3a} is H or C₁-C₆ alkyl. In some embodiments, the invention relates to a compound of formula (I), or a pharmaceutically acceptable salt thereof, wherein R^{3a} is H or —CH₃. In some embodiments, the invention relates to a compound of formula (I), or a pharmaceutically acceptable salt thereof, wherein R^{3a} is H. In some embodiments, the invention relates to a compound of formula (I), or a pharmaceutically acceptable salt thereof, wherein R^{3a} is —CH₃.

In some embodiments, the invention relates to a compound of formula (I), or a pharmaceutically acceptable salt thereof, wherein R^{4a} is H.

In some embodiments, the invention relates to a compound of formula (I), or a pharmaceutically acceptable salt thereof, wherein R^{6a} is H, —CN, C₁-C₆ alkoxy, —NH(C₁-C₆ alkyl), —N(C₁-C₆ alkyl)₂, —NHC(O)NH₂, —NHC(O)NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′}; and each R^{a′} is independently Cl, —OH, —CH₃, —CH₂CH₃, —CF₃, —C(O)OCH₂CH₃, or —C(O)NH₂. In some embodiments, the invention relates to a compound of formula (I), or a pharmaceutically acceptable salt thereof, wherein R^{6a} is H, —CN, C₁-C₆ alkoxy, —NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂. In other embodiments, R^{6a} is C₁-C₆ alkoxy. In other embodiments, R^{6a} is —NH(C₁-C₆ alkyl). In other embodiments, R^{6a} is —O(C₁-C₆ alkylene)-(C₁-C₆ alkoxy). In other embodiments, R^{6a} is —O(C₁-C₆ alkylene)-N(C₁-C₆ alkyl)₂. In other embodiments, R^{6a} is —C(O)NH(C₁-C₆ alkyl). In other embodiments, R^{6a} is —C(O)N(C₁-C₆ alkyl)₂. In other embodiments, R^{6a} is —C(O)O(C₁-C₆ alkyl). In other embodiments, R^{6a} is —N(C₁-C₆ alkyl)₂. In other embodiments, R^{6a} is —NHC(O)NH₂. In other embodiments, R^{6a} is —NHC(O)NH(C₁-C₆ alkyl). In other embodiments, R^{6a} is —C(NH)NH₂. In other embodiments, R^{6a} is —SO₂NH₂. In other embodiments, R^{6a} is 5-10 membered heteroaryl comprising 1-3 heteroatoms selected from nitrogen, oxygen, and sulfur, wherein said heteroaryl is optionally substituted with 1-2 R^{a′}; and each R^{a′} is independently Cl, —OH, —CH₃, —CH₂CH₃, —CF₃, —C(O)OCH₂CH₃, or —C(O)NH2. In some embodiments, the invention relates to a compound of formula (I), or a pharmaceutically acceptable salt thereof, wherein R^{6a} is H, —CN, —OCH₃, —OCH₂CH₂CH(CH₃)₂, —NH(CH₃), —N(CH₃)₂, —NHC(O)NH₂, —NHC(O)NHCH₃, —OH, —OCH₂CH₂OCH₃, —OCH₂CH₂N(CH₃)₂, —C(O)NH₂, —C(O)NH(CH₃), —C(O)N(CH₃)₂, —C(O)OH, —C(O)OCH₃, —C(NH)NH₂, —C(S)NH₂, —SO₂NH₂,

In some embodiments, the invention relates to a compound of formula (I), or a pharmaceutically acceptable salt thereof, wherein R^{6a} is H, —CN, —OCH₃, —OCH₂CH₂CH(CH₃)₂, —NH(CH₃), —OH, —OCH₂CH₂OCH₃, —OCH₂CH₂N(CH₃)₂, —C(O)NH₂, —C(O)NH(CH₃), —C(O)N(CH₃)₂, —C(O)OH, —C(O)OCH₃, or —C(S)NH₂. In other embodiments, R^{6a} is H. In other embodiments, R^{6a} is —CN. In other embodiments, R^{6a} is —OCH₃. In other embodiments, R^{6a} is —OCH₂CH₂CH(CH₃)₂. In other embodiments, R^{6a} is —NH(CH₃). In other embodiments, R^{6a} is —OH. In other embodiments, R^{6a} is —OCH₂CH₂OCH₃. In other embodiments, R^{6a} is —OCH₂CH₂N(CH₃)₂. In other embodiments, R^{6a} is —C(O)NH₂. In other embodiments, R^{6a} is —C(O)NH(CH₃). In other embodiments, R^{6a} is —C(O)N(CH₃)₂. In other embodiments, R^{6a} is —C(O)OH. In other embodiments, R^{6a} is —C(O)OCH₃. In other embodiments, R^{6a} is —C(S)NH₂. In other embodiments, R^{6a} is —N(CH₃)₂. In other embodiments, R^{6a} is —NHC(O)NH₂. In other embodiments, R^{6a} is —NHC(O)NHCH₃. In other embodiments, R^{6a} is —C(NH)NH₂. In other embodiments, R^{6a} is —SO₂NH₂. In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In some embodiments, the invention relates to a compound of formula (I), or a pharmaceutically acceptable salt thereof, wherein Y is

In some embodiments, the invention relates to a compound of formula (I), or a pharmaceutically acceptable salt thereof, wherein Y is

In some embodiments, the invention relates to a compound of formula (I), or a pharmaceutically acceptable salt thereof, wherein Y is

In some embodiments, the invention relates to a compound of formula (I), or a pharmaceutically acceptable salt thereof, wherein Y is

In some embodiments, the invention relates to a compound of formula (I), or a pharmaceutically acceptable salt thereof, wherein Y is

In some embodiments, the invention relates to a compound of formula (I), or a pharmaceutically acceptable salt thereof, wherein X_{3b} is N. In some embodiments, the invention relates to a compound of formula (I), or a pharmaceutically acceptable salt thereof, wherein X_{3b} is CR^{3b}. In some embodiments, the invention relates to a compound of formula (I), or a pharmaceutically acceptable salt thereof, wherein X_{3b} is CR^{3b} and R^{3b} is H, halo, C₁-C₆ alkyl, or C₁-C₆ haloalkyl. In some embodiments, the invention relates to a compound of formula (I), or a pharmaceutically acceptable salt thereof, wherein X_{3b} is CR^{3b} and R^{3b} is H, C₁-C₆ alkyl, or C₁-C₆ haloalkyl. In other embodiments, R^{3b} is C₁-C₆ alkyl. In other embodiments, R^{3b} is C₁-C₆ haloalkyl. In other embodiments, R^{3b} is halo. In some embodiments, the invention relates to a compound of formula (I), or a pharmaceutically acceptable salt thereof, wherein R^{3b} is H, F, —CH₃ or —CF₃. In some embodiments, the invention relates to a compound of formula (I), or a pharmaceutically acceptable salt thereof, wherein X_{3b} is CR^{3b} and R^{3b} is H, —CH₃, or —CF₃. In other embodiments, R^{3b} is H. In other embodiments, R^{3b} is —CH₃. In other embodiments, R^{3b} is —CF₃. In other embodiments, R^{3b} is D. In other embodiments, R^{3b} is —C(CD₃)₃. In other embodiments, R^{3b} is F.

In some embodiments, the invention relates to a compound of formula (I), or a pharmaceutically acceptable salt thereof, wherein X_{6b} is N. In some embodiments, the invention relates to a compound of formula (I), or a pharmaceutically acceptable salt thereof, wherein X_{6b} is CR^{6b}. In some embodiments, the invention relates to a compound of formula (I), or a pharmaceutically acceptable salt thereof, wherein X_{6b} is CR^{6b} and R^{6b} is H, halo, or C₁-C₆ alkyl. In other embodiments, R^{6b} is halo. In other embodiments, R^{6b} is C₁-C₆ alkyl. In some embodiments, the invention relates to a compound of formula (I), or a pharmaceutically acceptable salt thereof, wherein X_{6b} is CR^{6b} and R^{6b} is H, F, or —CH₃. In other embodiments, R^{6b} is H. In other embodiments, R^{6b} is F. In other embodiments, R^{6b} is —CH₃. In other embodiments, R^{6b} is D. In other embodiments, R^{6b} is —C(CD₃)₃.

In some embodiments, the invention relates to a compound of formula (I), or a pharmaceutically acceptable salt thereof, wherein R^{2b} is H, halo, C₁-C₆ alkyl, C₁-C₆ alkoxy, —(C₁-C₆ alkylene)-OH, or —C(O)O(C₁-C₆ alkyl). In some embodiments, the invention relates to a compound of formula (I), or a pharmaceutically acceptable salt thereof, wherein R^{2b} is H, halo, C₁-C₆ alkyl, C₁-C₆ alkoxy, or —(C₁-C₆ alkylene)-OH. In other embodiments, R^{2b} is halo. In other embodiments, R^{2b} is C₁-C₆ alkyl. In other embodiments, R^{2b} is C₁-C₆ alkoxy. In other embodiments, R^{2b} is —(C₁-C₆ alkylene)-OH. In other embodiments, R^{2b} is —C(O)O(C₁-C₆ alkyl). In some embodiments, the invention relates to a compound of formula (I), or a pharmaceutically acceptable salt thereof, wherein R^{2b} is H, F, Cl, —CH₃, —CH₂CH₃, —OCH₃, —CH₂OH, —CH₂CH₂CH₂OH, or —C(O)OCH₃. In some embodiments, the invention relates to a compound of formula (I), or a pharmaceutically acceptable salt thereof, wherein R^{2b} is H, F, Cl, —CH₃, —CH₂CH₃, —OCH₃, or —CH₂CH₂CH₂OH. In other embodiments, R^{2b} is H. In other embodiments, R^{2b} is F. In other embodiments, R^{2b} is Cl. In other embodiments, R^{2b} is —CH₃. In other embodiments, R^{2b} is —CH₂CH₃. In other embodiments, R^{2b} is —OCH₃. In other embodiments, R^{2b} is —CH₂CH₂CH₂OH. In other embodiments, R^{2b} is D. In other embodiments, R^{2b} is —C(CD₃)₃. In other embodiments, R^{2b} is —CH₂OH. In other embodiments, R^{2b} is —C(O)OCH₃.

In some embodiments, the invention relates to a compound of formula (I), or a pharmaceutically acceptable salt thereof, wherein R^{4b} is H, halo, C₁-C₆ alkyl, C₁-C₆ alkenyl, C₁-C₆ haloalkyl, C₁-C₆ haloalkenyl, C₁-C₆ haloalkoxy, —(C₁-C₆ alkylene)-OH, —(C₁-C₆ haloalkylene)-OH, —(C₁-C₆ alkylene)-C(O)OH, —(C₁-C₆ haloalkylene)-C(O)OH, —C(O)O(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I), or a pharmaceutically acceptable salt thereof, wherein R^{4b} is H, halo, C₁-C₆ alkyl, C₁-C₆ haloalkyl, C₁-C₆ haloalkenyl, —(C₁-C₆ alkylene)-OH, —(C₁-C₆ alkylene)-C(O)OH, —Si(C₁-C₆ alkyl)₃, or (C₁-C₆ haloalkyl)-(C₃-C₆ cycloalkyl)-. In other embodiments, R^{4b} is halo. In other embodiments, R^{4b} is C₁-C₆ alkyl. In other embodiments, R^{4b} is C₁-C₆ haloalkyl. In other embodiments, R^{4b} is C₁-C₆ haloalkenyl. In other embodiments, R^{4b} is —(C₁-C₆ alkylene)-OH. In other embodiments, R^{4b} is —(C₁-C₆ alkylene)-C(O)OH. In other embodiments, R^{4b} is —Si(C₁-C₆ alkyl)₃. In other embodiments, R^{4b} is (C₁-C₆ haloalkyl)-(C₃-C₆ cycloalkyl)-. In other embodiments, R^{4b} is C₁-C₆ alkenyl. In other embodiments, R^{4b} is C₁-C₆ haloalkoxy. In other embodiments, R^{4b} is —(C₁-C₆ haloalkylene)-OH. In other embodiments, R^{4b} is —(C₁-C₆ haloalkylene)-C(O)OH. In other embodiments, R^{4b} is —C(O)O(C₁-C₆ alkyl). In other embodiments, R^{4b} is C₆-C₁₀ aryl. In other embodiments, R^{4b} is C₃-C₁₀ cycloalkyl. In other embodiments, R^{4b} is (C₁-C₆ alkyl)-(C₃-C₆ cycloalkyl)-. In other embodiments, R^{4b} is 4-10 membered heterocyclyl comprising 1-3 heteroatoms selected from nitrogen and oxygen. In other embodiments, the cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I), or a pharmaceutically acceptable salt thereof, wherein R^{4b} is H, Cl, —CH₃, —CH(CH₃)₂, —C(CH₃)₃, —C(CH₃)₂(CH₂CH₃), —CF₃, —C(CH₃)₂(CHF₂), —C(CH₃)₂(CF₃), —CH₂C(CH₃)₂F, —C(═CH₂)(CF₃), —CH═C(CH₃)₂, —OCF₃, —C(CH₃)₂(CH₂OH), —C(CH₃)(CF₃)(CH₂OH), —C(CH₃)₂(C(O)OH), —C(CH₃)(CF₃)(C(O)OH), —C(O)OCH₃, —Si(CH₃)₃, phenyl, 1-methylcyclopropyl, 1-trifluoromethylcyclopropyl, cyclobutyl, 1-methylcyclobutyl, 3,3-difluoro-1-methylcyclobutyl, 4,4-difluoro-1-methylcyclohexyl,

In some embodiments, the invention relates to a compound of formula (I), or a pharmaceutically acceptable salt thereof, wherein R^{4b} is H, Cl, —CH₃, —CH(CH₃)₂, —C(CH₃)₃, —C(CH₃)₂(CH₂CH₃), —CF₃, —C(CH₃)₂(CF₃), —C(═CH₂)(CF₃), —C(CH₃)₂(CH₂OH), —C(CH₃)₂(C(O)OH), —Si(CH₃)₃, or 1-trifluoromethylcyclopropyl. In other embodiments, R^{4b} is H. In other embodiments, R^{4b} is Cl. In other embodiments, R^{4b} is —CH₃. In other embodiments, R^{4b} is —CH(CH₃)₂. In other embodiments, R^{4b} is —C(CH₃)₃. In other embodiments, R^{4b} is —C(CH₃)₂(CH₂CH₃). In other embodiments, R^{4b} is —CF₃. In other embodiments, R^{4b} is —C(CH₃)₂(CF₃). In other embodiments, R^{4b} is —C(═CH₂)(CF₃). In other embodiments, R^{4b} is —C(CH₃)₂(CH₂OH). In other embodiments, R^{4b} is —C(CH₃)₂(C(O)OH). In other embodiments, R^{4b} is —Si(CH₃)₃. In other embodiments, R^{4b} is 1-trifluoromethylcyclopropyl. In other embodiments, R^{4b} is D. In other embodiments, R^{4b} is —C(CD₃)₃. In other embodiments, R^{4b} is 1-(methyl-d₃)cyclopropyl. In other embodiments, R^{4b} is —C(CD₃)(CH₃)(CF₃). In other embodiments, R^{4b} is —C(¹³CD₃)(CH₃)(CF₃). In other embodiments, R^{4b} is —C(CD₃)₂(CD₂OH). In some embodiments, the invention relates to a compound of formula (I), or a pharmaceutically acceptable salt thereof, wherein R^{5b} is H, halo, C₁-C₆ alkyl, C₁-C₆ haloalkyl, C₁-C₆ alkoxy, or C₃-C₆ cycloalkyl. In other embodiments, R^{5b} is halo. In other embodiments, R^{5b} is C₁-C₆ alkyl. In other embodiments, R^{5b} is C₁-C₆ haloalkyl. In other embodiments, R^{5b} is C₁-C₆ alkoxy. In other embodiments, R^{5b} is C₃-C₆ cycloalkyl. In some embodiments, the invention relates to a compound of formula (I), or a pharmaceutically acceptable salt thereof, wherein R^{5b} is H, F, Cl, —CH₃, —CH₂CH₃, —CH(CH₃)₂, —C(CH₃)₃, —CF₃, —OCH₃, or cyclopropyl. In other embodiments, R^{5b} is H. In other embodiments, R^{5b} is F. In other embodiments, R^{5b} is Cl. In other embodiments, R^{5b} is —CH₃. In other embodiments, R^{5b} is —CH₂CH₃. In other embodiments, R^{5b} is —CH(CH₃)₂. In other embodiments, R^{5b} is —C(CH₃)₃. In other embodiments, R^{5b} is —CF₃. In other embodiments, R^{5b} is —OCH₃. In other embodiments, R^{5b} is cyclopropyl. In other embodiments, R^{5b} is D. In other embodiments, R^{5b} is —C(CD₃)³. In other embodiments, R^{5b} is —C(CH₃)₂(CHF₂). In other embodiments, R^{5b} is —CH₂C(CH₃)₂F. In other embodiments, R^{5b} is —CH═C(CH₃)₂. In other embodiments, R^{5b} is —OCF₃. In other embodiments, R^{5b} is —C(CH₃)(CF₃)(CH₂OH). In other embodiments, R^{5b} is —C(CH₃)(CF₃)(C(O)OH). In other embodiments, R^{5b} is —C(O)OCH₃. In other embodiments, R^{5b} is phenyl. In other embodiments, R^{5b} is 1-methylcyclopropyl. In other embodiments, R^{5b} is cyclobutyl. In other embodiments, R^{5b} is 1-methylcyclobutyl. In other embodiments, R^{5b} is 3,3-difluoro-1-methylcyclobutyl. In other embodiments, R^{5b} is cyclopentyl. In other embodiments, R^{5b} is 1-methylcyclopentyl. In other embodiments, R^{5b} is 1-trifluoromethylcyclopentyl. In other embodiments, R^{5b} is 3,3-difluoro-1-methylcyclopentyl. In other embodiments, R^{5b} is 4,4-difluoro-1-methylcyclohexyl. In other embodiments, R^{5b} is

In other embodiments, R^{5b} is

In other embodiments, R^{5b} is

In other embodiments, R^{5b} is

In other embodiments, R^{5b} is

In other embodiments, R^{5b} is

In other embodiments, R^{5b} is

In other embodiments, R^{5b} is

In other embodiments, R^{5b} is

In other embodiments, R^{5b} is

In other embodiments, R^{5b} is

In other embodiments, R^{5b} is

In other embodiments, R^{5b} is

In other embodiments, R^{5b} is

In other embodiments, R^{5b} is

In some embodiments, the invention relates to a compound of formula (I), or a pharmaceutically acceptable salt thereof, wherein X_{2b} is CR^{2b}. In some embodiments, the invention relates to a compound of formula (I), or a pharmaceutically acceptable salt thereof, wherein X_{2b} is CR^{2b} and R^{2b} is H or C₁-C₆ alkyl. In other embodiments, R^{2b} is C₁-C₆ alkyl. In some embodiments, the invention relates to a compound of formula (I), or a pharmaceutically acceptable salt thereof, wherein X_{2b} is CR^{2b} and R^{2b} is H or —CH₃. In other embodiments, R^{2b} is H. In other embodiments, R^{2b} is —CH₃.

In some embodiments, the invention relates to a compound of formula (I), or a pharmaceutically acceptable salt thereof, wherein X_{5b} is N. In some embodiments, the invention relates to a compound of formula (I), or a pharmaceutically acceptable salt thereof, wherein X_{5b} is CR^{5b}. In some embodiments, the invention relates to a compound of formula (I), or a pharmaceutically acceptable salt thereof, wherein X_{5b} is CR^{5b} and R^{5b} is H or C₁-C₆ alkyl. In other embodiments, R^{5b} is C₁-C₆ alkyl. In some embodiments, the invention relates to a compound of formula (I), or a pharmaceutically acceptable salt thereof, wherein X_{5b} is CR^{5b} and R^{5b} is H or —CH₃. In other embodiments, R^{5b} is H. In other embodiments, R^{5b} is —CH₃.

In some embodiments, the invention relates to a compound of formula (I), or a pharmaceutically acceptable salt thereof, wherein X_{6b} is CR^{6b}. In some embodiments, the invention relates to a compound of formula (I), or a pharmaceutically acceptable salt thereof, wherein X_{6b} is CR^{6b} and R^{6b} is H, C₁-C₆ alkyl, —OH, or —N(C₁-C₆ alkyl)₂. In other embodiments, R^{6b} is C₁-C₆ alkyl. In other embodiments, R^{6b} is —N(C₁-C₆ alkyl)₂. In some embodiments, the invention relates to a compound of formula (I), or a pharmaceutically acceptable salt thereof, wherein X₆, is CR^{6b} and R^{6b} is H, —CH₃, —OH, —N(CH₃)₂, —N(CH₂CH₂CH₂)₃, —N(CH₃)(CH₂CH₂CH₂CH₃), —N(CH₂CH₃)(CH₂CH₂CH₂CH₃), or —N(CH₂CH(CH₃)₂)₂. In other embodiments, R^{6b} is H. In other embodiments, R^{6b} is —CH₃. In other embodiments, R^{6b} is —OH. In other embodiments, R^{6b} is —N(CH₃)₂. In other embodiments, R^{6b} is —N(CH₂CH₂CH₂)₃. In other embodiments, R^{6b} is —N(CH₃)(CH₂CH₂CH₂CH₃). In other embodiments, R^{6b} is —N(CH₂CH₃)(CH₂CH₂CH₂CH₃). In other embodiments, R^{6b} is —N(CH₂CH(CH₃)₂)₂.

In some embodiments, the invention relates to a compound of formula (I), or a pharmaceutically acceptable salt thereof, wherein Z is a 5-6 membered aromatic or nonaromatic carbocycle optionally substituted with one to four R^{z}. In some embodiments, the invention relates to a compound of formula (I), or a pharmaceutically acceptable salt thereof, wherein Z is

In other embodiments, Z is

In other embodiments, Z is

In other embodiments, Z is

In other embodiments, Z is

In other embodiments, Z is

In other embodiments, Z is

In other embodiments, Z is

In other embodiments, Z is

In other embodiments, Z is

In some embodiments, the invention relates to a compound of formula (I), or a pharmaceutically acceptable salt thereof, wherein R^{z} is C₁-C₆ alkyl or C₁-C₆ haloalkyl. In other embodiments, R^{z} is C₁-C₆ alkyl. In other embodiments, R^{z} is C₁-C₆ haloalkyl. In some embodiments, the invention relates to a compound of formula (I), or a pharmaceutically acceptable salt thereof, wherein R^{z} is —CH₃ or —CF₃. In other embodiments, R^{z} is —CH₃. In other embodiments, R^{z} is —CF₃.

In some embodiments, the invention relates to a compound of formula (I), or a pharmaceutically acceptable salt thereof, wherein R^{7b} is —CH₃ or —CH(CH₃)₂. In other embodiments, R^{7b} is —CH₃. In other embodiments, R^{7b} is —CH(CH₃)₂.

In some embodiments, the invention relates to a compound of formula (I), or a pharmaceutically acceptable salt thereof, wherein R^{9b} is —C(CH₃)₃.

In some embodiments, the invention relates to a compound of formula (I), or a pharmaceutically acceptable salt thereof, wherein R^{10b} is H or Cl. In other embodiments, R^{10b} is H. In other embodiments, R^{10b} is Cl.

In some embodiments, the invention relates to a compound of formula (I-A):

or a pharmaceutically acceptable salt thereof, wherein:
X₅ₐ is N or N⁺—O⁻;
X₉ₐ is N or CR^{9a};
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —NHC(O)NH₂, —NHC(O)NH(C₁-C₆ alkyl), —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′};
R^{9a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
each R^{a′} is independently halo, —OH, C₁-C₆ alkyl, C₁-C₆ haloalkyl, —C(O)O(C₁-C₆ alkyl), or —C(O)NH₂;
Y is

X_{2b} is N or CR^{2b};
X_{3b} is N or CR^{3b};
X_{4b} is N or CR^{4b};
X_{5b} is N or CR^{5b};
X_{6b} is N or CR^{6b};
Z is a 5-7 membered aromatic or nonaromatic ring optionally containing 1-3 heteroatoms selected from nitrogen and oxygen and optionally substituted with one or more R^{z};
R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{3b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ haloalkylene)-OH, —(C₁-C₆ haloalkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′};
R^{5b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{6b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)(C₂-C₆ alkyl), —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{7b} is C₁-C₆ alkyl;
R^{9b} is C₁-C₆ alkyl;
R^{10b} is H or halo;
each R^{b′} is independently C₁-C₆ alkyl or C₁-C₆ haloalkyl; and
R^{z} is halo, C₁-C₆ alkyl, C₁-C₆ alkenyl, C₁-C₆ alkoxy, C₁-C₆ haloalkenyl, —CN, —C(O)NH₂, —C(O)NH(C₁-C₆ alkyl), —C(O)N(C₁-C₆ alkyl)₂, or —C(O)OH;
   provided that:
(i) if Y is

X_{3b} is CR^{3b}, and X_{6b} is CR^{6b}, then no more than three of R^{2b}, R^{3b}, R^{4b}, R^{5b}, and R^{6b} are H; and
(ii) if Y is

X_{3b} is N, and X_{6b} is CR^{6b}, then no more than three of R^{2b}, R^{4b}, R^{5b}, and R^{6b} are H; and
(iii) if Y is

X_{3b} is CR^{3b}, and X_{6b} is N, then no more than three of R^{2b}, R^{3b}, R^{4b}, and R^{5b} are H; and
(iv) if X₉ₐ is

X_{3b} is CR^{3b}, and X_{6b} is CR^{6b}, then no more than two of R^{2b}, R^{3b}, R^{4b}, R^{5b}, and R^{6b} are halo; and
(v) if X₉ₐ is

X_{4b} is CR^{4b}, X_{5b} is CR^{5b}, and X_{6b} is CR^{6b}, then:
   no more than two of R^{4b}, R^{5b}, and R^{6b} are H; or
   Z is substituted with one or more R^{z}.

In some embodiments, the invention relates to a compound of formula (I-A), or a pharmaceutically acceptable salt thereof, wherein:
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
Y is

R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo.

In some embodiments, the invention relates to a compound of formula (I-A), or a pharmaceutically acceptable salt thereof, wherein X₅ₐ is N. In some embodiments, the invention relates to a compound of formula (I-A), or a pharmaceutically acceptable salt thereof, wherein X₅ₐ is N⁺—O⁻.

In some embodiments, the invention relates to a compound of formula (I-A), or a pharmaceutically acceptable salt thereof, wherein X₉ₐ is CR^{9a}. In some embodiments, the invention relates to a compound of formula (I-A), or a pharmaceutically acceptable salt thereof, wherein X₉ₐ is CR^{9a} and R^{9a} is H, halo, C₁-C₆ alkyl, C₁-C₆ alkoxy, or —C(O)O(C₁-C₆ alkyl). In some embodiments, the invention relates to a compound of formula (I-A), or a pharmaceutically acceptable salt thereof, wherein X₉ₐ is CR^{9a} and R^{9a} is H, halo, C₁-C₆ alkyl, or C₁-C₆ alkoxy. In other embodiments, R^{9a} is halo. In other embodiments, R^{9a} is C₁-C₆ alkyl. In other embodiments, R^{9a} is C₁-C₆ alkoxy. In other embodiments, R^{9a} is —C(O)O(C₁-C₆ alkyl). In some embodiments, the invention relates to a compound of formula (I-A), or a pharmaceutically acceptable salt thereof, wherein R^{9a} is H, Cl, Br, —CH₃, —OCH₃, —OCH₂CH₃, or —C(O)OCH₃. In some embodiments, the invention relates to a compound of formula (I-A), or a pharmaceutically acceptable salt thereof, wherein X₉ₐ is CR^{9a} and R^{9a} is H, Cl, Br, —CH₃, —OCH₃, or —OCH₂CH₃. In other embodiments, R^{9a} is H. In other embodiments, R^{9a} is Cl. In other embodiments, R^{9a} is Br. In other embodiments, R^{9a} is —CH₃. In other embodiments, R^{9a} is —OCH₃. In other embodiments, R^{9a} is —OCH₂CH₃. In other embodiments, R^{9a} is —C(O)OCH₃.

In some embodiments, the invention relates to a compound of formula (I-A), or a pharmaceutically acceptable salt thereof, wherein R^{6a} is H, —CN, C₁-C₄ alkoxy, —NH(C₁-C₆ alkyl), —N(C₁-C₆ alkyl)₂, —NHC(O)NH₂, —NHC(O)NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′}; and each R^{a′} is independently Cl, —OH, —CH₃, —CH₂CH₃, —CF₃, —C(O)OCH₂CH₃, or —C(O)NH₂. In some embodiments, the invention relates to a compound of formula (I-A), or a pharmaceutically acceptable salt thereof, wherein R^{6a} is H, —CN, C₁-C₄ alkoxy, —NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂. In other embodiments, R^{6′} is H. In other embodiments, R^{6a} is C₁-C₄ alkoxy. In other embodiments, R^{6a} is —NH(C₁-C₆ alkyl). In other embodiments, R^{6a} is —O(C₁-C₆ alkylene)-(C₁-C₆ alkoxy). In other embodiments, R^{6a} is —O(C₁-C₆ alkylene)-N(C₁-C₆ alkyl)₂. In other embodiments, R^{6a} is —C(O)NH(C₁-C₆ alkyl). In other embodiments, R^{6a} is —C(O)N(C₁-C₆ alkyl)₂. In other embodiments, R^{6a} is —C(O)O(C₁-C₆ alkyl). In other embodiments, R^{6a} is —N(C₁-C₆ alkyl)₂. In other embodiments, R^{6a} is —NHC(O)NH₂. In other embodiments, R^{6a} is —NHC(O)NH(C₁-C₆ alkyl). In other embodiments, R^{6a} is —C(NH)NH₂. In other embodiments, R^{6a} is —SO₂NH₂. In other embodiments, R^{6a} is 5-10 membered heteroaryl comprising 1-3 heteroatoms selected from nitrogen, oxygen, and sulfur, wherein said heteroaryl is optionally substituted with 1-2 R^{a′}; and each R^{a′} is independently Cl, —OH, —CH₃, —CH₂CH₃, —CF₃, —C(O)OCH₂CH₃, or —C(O)NH₂. In some embodiments, the invention relates to a compound of formula (I-A), or a pharmaceutically acceptable salt thereof, wherein R^{6a} is H, —CN, —OCH₃, —NH(CH₃), —N(CH₃)₂, —NHC(O)NH₂, —NHC(O)NHCH₃, —OH, —OCH₂CH₂OCH₃, —OCH₂CH₂N(CH₃)₂, —C(O)NH₂, —C(O)NH(CH₃), —C(O)N(CH₃)₂, —C(O)OH, —C(O)OCH₃, —C(NH)NH₂, —C(S)NH₂, —SO₂NH₂,

In some embodiments, the invention relates to a compound of formula (I-A), or a pharmaceutically acceptable salt thereof, wherein R^{6a} is H, —CN, —OCH₃, —NH(CH₃), —OH, —OCH₂CH₂OCH₃, —OCH₂CH₂N(CH₃)₂, —C(O)NH₂, —C(O)NH(CH₃), —C(O)N(CH₃)₂, —C(O)OH, —C(O)OCH₃, or —C(S)NH₂. In other embodiments, R^{6a} is H. In other embodiments, R^{6a} is —CN. In other embodiments, R^{6a} is —OCH₃. In other embodiments, R^{6a} is —NH(CH₃). In other embodiments, R^{6a} is —OH. In other embodiments, R^{6a} is —OCH₂CH₂OCH₃. In other embodiments, R^{6a} is —OCH₂CH₂N(CH₃)₂. In other embodiments, R^{6a} is —C(O)NH₂. In other embodiments, R^{6a} is —C(O)NH(CH₃). In other embodiments, R^{6a} is —C(O)N(CH₃)₂. In other embodiments, R^{6a} is —C(O)OH. In other embodiments, R^{6a} is —C(O)OCH₃. In other embodiments, R^{6a} is —C(S)NH₂. In other embodiments, R^{6a} is —N(CH₃)₂. In other embodiments, R^{6a} is —NHC(O)NH₂. In other embodiments, R^{6a} is —NHC(O)NHCH₃. In other embodiments, R^{6a} is —C(NH)NH₂. In other embodiments, R^{6a} is —SO₂NH₂. In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In some embodiments, the invention relates to a compound of formula (I-A), or a pharmaceutically acceptable salt thereof, wherein Y is

In some embodiments, the invention relates to a compound of formula (I-A), or a pharmaceutically acceptable salt thereof, wherein Y is

In other embodiments, the invention relates to a compound of formula (I-A), or a pharmaceutically acceptable salt thereof, wherein Y is

In some embodiments, the invention relates to a compound of formula (I-A), or a pharmaceutically acceptable salt thereof, wherein Y is

In some embodiments, the invention relates to a compound of formula (I-A), or a pharmaceutically acceptable salt thereof, wherein Y is

In some embodiments, the invention relates to a compound of formula (I-A), or a pharmaceutically acceptable salt thereof, wherein X_{3b} is N. In some embodiments, the invention relates to a compound of formula (I-A), or a pharmaceutically acceptable salt thereof, wherein X_{3b} is CR^{3b}. In some embodiments, the invention relates to a compound of formula (I-A), or a pharmaceutically acceptable salt thereof, wherein X_{3b} is CR^{3b} and R^{3b} is H, halo, C₁-C₆ alkyl, or C₁-C₆ haloalkyl. In some embodiments, the invention relates to a compound of formula (I-A), or a pharmaceutically acceptable salt thereof, wherein X_{3b} is CR^{3b} and R^{3b} is H, C₁-C₆ alkyl, or C₁-C₆ haloalkyl. In other embodiments, R^{3b} is C₁-C₆ alkyl. In other embodiments, R^{3b} is C₁-C₆ haloalkyl. In other embodiments, R^{3b} is halo. In some embodiments, the invention relates to a compound of formula (I-A), or a pharmaceutically acceptable salt thereof, wherein R^{3b} is H, F, —CH₃, or —CF₃. In some embodiments, the invention relates to a compound of formula (I-A), or a pharmaceutically acceptable salt thereof, wherein R^{3b} is H, —CH₃, or —CF₃. In other embodiments, R^{3b} is H. In other embodiments, R^{3b} is —CH₃. In other embodiments, R^{3b} is —CF₃. In other embodiments, R^{3b} is D. In other embodiments, R^{3b} is —C(CD₃)₃. In other embodiments, R^{3b} is F.

In some embodiments, the invention relates to a compound of formula (I-A), or a pharmaceutically acceptable salt thereof, wherein X_{6b} is N. In some embodiments, the invention relates to a compound of formula (I-A), or a pharmaceutically acceptable salt thereof, wherein X_{6b} is CR^{6b}. In some embodiments, the invention relates to a compound of formula (I-A), or a pharmaceutically acceptable salt thereof, wherein X_{6b} is CR^{6b} and R^{6b} is H, halo, or C₁-C₆ alkyl. In other embodiments, R^{6b} is halo. In other embodiments, R^{6b} is C₁-C₆ alkyl. In some embodiments, the invention relates to a compound of formula (I-A), or a pharmaceutically acceptable salt thereof, wherein X_{6b} is CR^{6b} and R^{6b} is H, F, or —CH₃. In other embodiments, R^{6b} is H. In other embodiments, R^{6b} is F. In other embodiments, R^{6b} is —CH₃. In other embodiments, R^{6b} is D. In other embodiments, R^{6b} is —C(CD₃)₃.

In some embodiments, the invention relates to a compound of formula (I-A), or a pharmaceutically acceptable salt thereof, wherein R^{2b} is H, halo, C₁-C₆ alkyl, C₁-C₆ alkoxy, —(C₁-C₆ alkylene)-OH, or —C(O)O(C₁-C₆ alkyl). In some embodiments, the invention relates to a compound of formula (I-A), or a pharmaceutically acceptable salt thereof, wherein R^{2b} is H, halo, C₁-C₆ alkyl, C₁-C₆ alkoxy, or —(C₁-C₆ alkylene)-OH. In other embodiments, R^{2b} is halo. In other embodiments, R^{2b} is C₁-C₆ alkyl. In other embodiments, R^{2b} is C₁-C₆ alkoxy. In other embodiments, R^{2b} is —(C₁-C₆ alkylene)-OH. In other embodiments, R^{2b} is —C(O)O(C₁-C₆ alkyl). In some embodiments, the invention relates to a compound of formula (I-A), or a pharmaceutically acceptable salt thereof, wherein R^{2b} is H, F, Cl, —CH₃, —CH₂CH₃, —OCH₃, —CH₂OH, —CH₂CH₂CH₂OH, or —C(O)OCH₃. In some embodiments, the invention relates to a compound of formula (I-A), or a pharmaceutically acceptable salt thereof, wherein R^{2b} is H, F, Cl, —CH₃, —CH₂CH₃, —OCH₃, or —CH₂CH₂CH₂OH. In other embodiments, R^{2b} is H. In other embodiments, R^{2b} is F. In other embodiments, R^{2b} is Cl. In other embodiments, R^{2b} is —CH₃. In other embodiments, R^{2b} is —CH₂CH₃. In other embodiments, R^{2b} is —OCH₃. In other embodiments, R^{2b} is —CH₂CH₂CH₂OH. In other embodiments, R^{2b} is D. In other embodiments, R^{2b} is —C(CD₃)₃. In other embodiments, R^{2b} is —CH₂OH. In other embodiments, R^{2b} is —C(O)OCH₃.

In some embodiments, the invention relates to a compound of formula (I-A), or a pharmaceutically acceptable salt thereof, wherein R^{4b} is H, halo, C₁-C₆ alkyl, C₁-C₆ alkenyl, C₁-C₆ haloalkyl, C₁-C₆ haloalkenyl, C₁-C₆ haloalkoxy, —(C₁-C₆ alkylene)-OH, —(C₁-C₆ haloalkylene)-OH, —(C₁-C₆ alkylene)-C(O)OH, —(C₁-C₆ haloalkylene)-C(O)OH, —C(O)O(C₁-C₆ alkyl), —Si(C₁-C₆ alkyl)₃, C₆-C₁₀ aryl, C₃-C₁₀ cycloalkyl, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I-A), or a pharmaceutically acceptable salt thereof, wherein R^{4b} is H, halo, C₁-C₆ alkyl, C₁-C₆ haloalkyl, C₁-C₆ haloalkenyl, —(C₁-C₆ alkylene)-OH, —Si(C₁-C₆ alkyl)₃, or (C₁-C₆ haloalkyl)-(C₃-C₆ cycloalkyl)-. In other embodiments, R^{4b} is halo. In other embodiments, R^{4b} is C₁-C₆ alkyl. In other embodiments, R^{4b} is C₁-C₆ haloalkyl. In other embodiments, R^{4b} is C₁-C₆ haloalkenyl. In other embodiments, R^{4b} is —(C₁-C₆ alkylene)-OH. In other embodiments, R^{4b} is —Si(C₁-C₆ alkyl)₃. In other embodiments, R^{4b} is (C₁-C₆ haloalkyl)-(C₃-C₆ cycloalkyl)-. In other embodiments, R^{4b} is C₁-C₆ alkenyl. In other embodiments, R^{4b} is C₁-C₆ haloalkoxy. In other embodiments, R^{4b} is —(C₁-C₆ haloalkylene)-OH. In other embodiments, R^{4b} is —(C₁-C₆ alkylene)-C(O)OH. In other embodiments, R^{4b} is —(C₁-C₆ haloalkylene)-C(O)OH. In other embodiments, R^{4b} is —C(O)O(C₁-C₆ alkyl). In other embodiments, R^{4b} is C₆-C₁₀ aryl. In other embodiments, R^{4b} is C₃-C₁₀ cycloalkyl. In other embodiments, R^{4b} is 4-10 membered heterocyclyl comprising 1-3 heteroatoms selected from nitrogen and oxygen. In other embodiments, the cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I-A), or a pharmaceutically acceptable salt thereof, wherein R^{4b} is H, Cl, —CH₃, —CH(CH₃)₂, —C(CH₃)₃, —C(CH₃)₂(CH₂CH₃), —CF₃, —C(CH₃)₂(CHF₂), —C(CH₃)₂(CF₃), —CH₂C(CH₃)₂F, —CH═C(CH₃)₂, —C(═CH₂)(CF₃), —OCF₃, —C(CH₃)₂(CH₂OH), —C(CH₃)(CF₃)(CH₂OH), —C(CH₃)₂(C(O)OH), —C(CH₃)(CF₃)(C(O)OH), —C(O)OCH₃, —Si(CH₃)₃, phenyl, 1-methylcyclopropyl, 4,4-difluoro-1-methylcyclohexyl,

In some embodiments, the invention relates to a compound of formula (I-A), or a pharmaceutically acceptable salt thereof, wherein R^{4b} is H, Cl, —CH₃, —CH(CH₃)₂, —C(CH₃)₃, —C(CH₃)₂(CH₂CH₃), —CF₃, —C(CH₃)₂(CF₃), —C(═CH₂)(CF₃), —C(CH₃)₂(CH₂OH), —Si(CH₃)₃, or 1-trifluoromethylcyclopropyl. In other embodiments, R^{4b} is H. In other embodiments, R^{4b} is Cl. In other embodiments, R^{4b} is —CH₃. In other embodiments, R^{4b} is —CH(CH₃)₂. In other embodiments, R^{4b} is —C(CH₃)₃. In other embodiments, R^{4b} is —C(CH₃)₂(CH₂CH₃). In other embodiments, R^{4b} is —CF₃. In other embodiments, R^{4b} is —C(CH₃)₂(CF₃). In other embodiments, R^{4b} is —C(═CH₂)(CF₃). In other embodiments, R^{4b} is —C(CH₃)₂(CH₂OH). In other embodiments, R^{4b} is —Si(CH₃)₃. In other embodiments, R^{4b} is 1-trifluoromethylcyclopropyl. In other embodiments, R^{4b} is D. In other embodiments, R^{4b} is —C(CD₃)₃. In other embodiments, R^{4b} is —C(CH₃)₂(CHF₂). In other embodiments, R^{4b} is —CH₂C(CH₃)₂F. In other embodiments, R^{4b} is —CH═C(CH₃)₂. In other embodiments, R^{4b} is —OCF₃. In other embodiments, R^{4b} is —C(CH₃)(CF₃)(CH₂OH). In other embodiments, R^{4b} is —C(CH₃)₂(C(O)OH). In other embodiments, R^{4b} is —C(CH₃)(CF₃)(C(O)OH). In other embodiments, R^{4b} is —C(O)OCH₃. In other embodiments, R^{4b} is phenyl. In other embodiments, R⁴ is 1-methylcyclopropyl. In other embodiments, R^{4b} is cyclobutyl. In other embodiments, R^{4b} is 1-methylcyclobutyl. In other embodiments, R^{4b} is 3,3-difluoro-1-methylcyclobutyl. In other embodiments, R^{4b} is cyclopentyl. In other embodiments, R^{4b} is 1-methylcyclopentyl. In other embodiments, R^{4b} is 1-trifluoromethylcyclopentyl. In other embodiments, R^{4b} is 3,3-difluoro-1-methylcyclopentyl. In other embodiments, R^{4b} is 4,4-difluoro-1-methylcyclohexyl. In other embodiments, R^{4b} is

In other embodiments, R^{4b} is

In other embodiments R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In some embodiments, the invention relates to a compound of formula (I-A), or a pharmaceutically acceptable salt thereof, wherein R^{5b} is H, halo, C₁-C₆ alkyl, C₁-C₆ haloalkyl, C₁-C₆ alkoxy, or C₃-C₆ cycloalkyl. In other embodiments, R^{5b} is halo. In other embodiments, R^{5b} is C₁-C₆ alkyl. In other embodiments, R^{5b} is C₁-C₆ haloalkyl. In other embodiments, R^{5b} is C₁-C₆ alkoxy. In other embodiments, R^{5b} is C₃-C₆ cycloalkyl. In some embodiments, the invention relates to a compound of formula (I-A), or a pharmaceutically acceptable salt thereof, wherein R^{5b} is H, F, Cl, —CH₃, —CH₂CH₃, —CH(CH₃)₂, —C(CH₃)₃, —CF₃, —OCH₃, or cyclopropyl. In other embodiments, R^{5b} is H. In other embodiments, R^{5b} is F. In other embodiments, R^{5b} is Cl. In other embodiments, R^{5b} is —CH₃. In other embodiments, R^{5b} is —CH₂CH₃. In other embodiments, R^{5b} is —CH(CH₃)₂. In other embodiments, R^{5b} is —C(CH₃)₃. In other embodiments, R^{5b} is —CF₃. In other embodiments, R^{5b} is —OCH₃. In other embodiments, R^{5b} is cyclopropyl. In other embodiments, R^{5b} is D. In other embodiments, R^{5b} is —C(CD₃)₃.

In some embodiments, the invention relates to a compound of formula (I-A), or a pharmaceutically acceptable salt thereof, wherein X_{2b} is CR^{2b}. In some embodiments, the invention relates to a compound of formula (I-A), or a pharmaceutically acceptable salt thereof, wherein X_{2b} is CR^{2b} and R^{2b} is H or C₁-C₆ alkyl. In other embodiments, R^{2b} is C₁-C₆ alkyl. In some embodiments, the invention relates to a compound of formula (I-A), or a pharmaceutically acceptable salt thereof, wherein X_{2b} is CR^{2b} and R^{2b} is H or —CH₃. In other embodiments, R^{2b} is H. In other embodiments, R^{2b} is —CH₃.

In some embodiments, the invention relates to a compound of formula (I-A), or a pharmaceutically acceptable salt thereof, wherein X_{5b} is N. In some embodiments, the invention relates to a compound of formula (I-A), or a pharmaceutically acceptable salt thereof, wherein X_{5b} is CR^{5b}. In some embodiments, the invention relates to a compound of formula (I-A), or a pharmaceutically acceptable salt thereof, wherein X_{5b} is CR^{5b} and R^{5b} is H or C₁-C₆ alkyl. In other embodiments, R^{5b} is C₁-C₆ alkyl. In some embodiments, the invention relates to a compound of formula (I-A), or a pharmaceutically acceptable salt thereof, wherein X₅, is CR^{5b} and R^{5b} is H or —CH₃. In other embodiments, R^{5b} is H. In other embodiments, R^{5b} is —CH₃.

In some embodiments, the invention relates to a compound of formula (I-A), or a pharmaceutically acceptable salt thereof, wherein X_{6b} is CR^{6b}. In some embodiments, the invention relates to a compound of formula (I-A), or a pharmaceutically acceptable salt thereof, wherein X_{6b} is CR^{6b} and R^{6b} is H, C₁-C₆ alkyl, or —N(C₁-C₆ alkyl)(C₂-C₆ alkyl). In other embodiments, R^{6b} is C₁-C₆ alkyl. In other embodiments, R^{6b} is —N(C₁-C₆ alkyl)(C₂-C₆ alkyl). In some embodiments, the invention relates to a compound of formula (I-A), or a pharmaceutically acceptable salt thereof, wherein X_{6b} is CR^{6b} and R^{6b} is H, —CH₃, —N(CH₃)(CH₂CH₂CH₂CH₃), —N(CH₂CH₃)(CH₂CH₂CH₃), —N(CH₂CH₂CH₃)₂, or —N(CH₂CH(CH₃)₂)₂. In other embodiments, R^{6b} is H. In other embodiments, R^{6b} is —CH₃. In other embodiments, R^{6b} is —N(CH₃)(CH₂CH₂CH₂CH₃). In other embodiments, R^{6b} is —N(CH₂CH₃)(CH₂CH₂CH₃). In other embodiments, R^{6b} is —N(CH₂CH₂CH₃)₂. In other embodiments, R^{6b} is —N(CH₂CH(CH₃)₂)₂.

In some embodiments, the invention relates to a compound of formula (I-A), or a pharmaceutically acceptable salt thereof, wherein Z is a 5-6 membered aromatic or nonaromatic carbocycle optionally substituted with one to four R^{z}. In some embodiments, the invention relates to a compound of formula (I-A), or a pharmaceutically acceptable salt thereof, wherein Z is

In other embodiments, Z is

In other embodiments Z is

In other embodiments, Z is

In other embodiments, Z is

In other embodiments, Z is

In other embodiments, Z is

In other embodiments, Z is

In other embodiments, Z is

In some embodiments, the invention relates to a compound of formula (I-A), or a pharmaceutically acceptable salt thereof, wherein R^{z} is C₁-C₆ alkyl. In some embodiments, the invention relates to a compound of formula (I-A), or a pharmaceutically acceptable salt thereof, wherein R^{z} is —CH₃.

In some embodiments, the invention relates to a compound of formula (I-A), or a pharmaceutically acceptable salt thereof, wherein R^{7b} is —CH₃ or —CH(CH₃)₂. In other embodiments, R^{7b} is —CH₃. In other embodiments, R^{7b} is —CH(CH₃)₂

In some embodiments, the invention relates to a compound of formula (I-A), or a pharmaceutically acceptable salt thereof, wherein R^{9b} is —C(CH₃)₃.

In some embodiments, the invention relates to a compound of formula (I-A), or a pharmaceutically acceptable salt thereof, wherein R^{10b} is H or Cl. In other embodiments, R^{10b} is H. In other embodiments, R^{10b} is Cl.

In some embodiments, the invention relates to a compound of formula (I-B):

or a pharmaceutically acceptable salt thereof, wherein:
X₅ₐ is N or N⁺—O⁻;
R^{3a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
R^{4a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —NHC(O)NH₂, —NHC(O)NH(C₁-C₆ alkyl), —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′};
R^{9a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
each R^{a′} is independently halo, —OH, C₁-C₆ alkyl, C₁-C₆ haloalkyl, —C(O)O(C₁-C₆ alkyl), or —C(O)NH₂;
Y is

X_{2b} is N or CR^{2b};
X_{3b} is N or CR^{3b};
X_{4b} is N or CR^{4b};
X_{5b} is N or CR^{5b};
X_{6b} is N or CR^{6b};
Z is a 5-7 membered aromatic or nonaromatic ring optionally containing 1-3 heteroatoms selected from nitrogen and oxygen and optionally substituted with one or more R^{z};
R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{3b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ haloalkylene)-OH, —(C₁-C₆ haloalkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′};
R^{5b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{6b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)(C₂-C₆ alkyl), —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{7b} is C₁-C₆ alkyl;
R^{9b} is C₁-C₆ alkyl;
R^{10b} is H or halo;
each R^{b′} is independently C₁-C₆ alkyl or C₁-C₆ haloalkyl; and
R^{z} is halo, C₁-C₆ alkyl, C₁-C₆ alkenyl, C₁-C₆ alkoxy, C₁-C₆ haloalkenyl, —CN, —C(O)NH₂, —C(O)NH(C₁-C₆ alkyl), —C(O)N(C₁-C₆ alkyl)₂, or —C(O)OH;
provided that:
(i) if Y is

X_{3b} is CR^{3b}, and X_{6b} is CR^{6b}, then no more than three of R^{2b}, R^{3b}, R^{4b}, R^{5b}, and R^{6b} are H.

In some embodiments, the invention relates to a compound of formula (I-B), or a pharmaceutically acceptable salt thereof, wherein:
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
Y is

R^{2b} is H, halo, C₁-C₆ alkyl, C₁-C₆ alkenyl, C₁-C₆ haloalkyl, C₁-C₆ haloalkenyl, i-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo.

In some embodiments, the invention relates to a compound of formula (I-B), or a pharmaceutically acceptable salt thereof, wherein X₅ₐ is N. In some embodiments, the invention relates to a compound of formula (I-B), or a pharmaceutically acceptable salt thereof, wherein X₅ₐ is N⁺—O⁻.

In some embodiments, the invention relates to a compound of formula (I-B), or a pharmaceutically acceptable salt thereof, wherein R^{3a} is H or C₁-C₆ alkyl. In some embodiments, the invention relates to a compound of formula (I-B), or a pharmaceutically acceptable salt thereof, wherein R^{3a} is H or —CH₃. In some embodiments, the invention relates to a compound of formula (I-B), or a pharmaceutically acceptable salt thereof, wherein R^{3a} is H. In other embodiments, R^{3a} is C₁-C₆ alkyl. In other embodiments, R^{3a} is —CH₃.

In some embodiments, the invention relates to a compound of formula (I-B), or a pharmaceutically acceptable salt thereof, wherein R^{4a} is H.

In some embodiments, the invention relates to a compound of formula (I-B), or a pharmaceutically acceptable salt thereof, wherein R^{6a} is H, —CN, C₁-C₄ alkoxy, —NH(C₁-C₆ alkyl), —N(C₁-C₆ alkyl)₂, —NHC(O)NH₂, —NHC(O)NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′}; and each R^{a′} is Cl, —OH, —CH₃, —CH₂CH₃, —CF₃, —C(O)OCH₂CH₃, or —C(O)NH2. In some embodiments, the invention relates to a compound of formula (I-B), or a pharmaceutically acceptable salt thereof, wherein R^{6a} is H, —CN, C₁-C₄ alkoxy, —NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂. In other embodiments, R^{6a} is C₁-C₄ alkoxy. In other embodiments, R^{6a} is —NH(C₁-C₆ alkyl). In other embodiments, R^{6a} is —O(C₁-C₆ alkylene)-(C₁-C₆ alkoxy). In other embodiments, R^{6a} is —O(C₁-C₆ alkylene)-N(C₁-C₆ alkyl)₂. In other embodiments, R^{6a} is —C(O)NH(C₁-C₆ alkyl). In other embodiments, R^{6a} is —C(O)N(C₁-C₆ alkyl)₂. In other embodiments, R^{6a} is —C(O)O(C₁-C₆ alkyl). In other embodiments, R^{6a} is —N(C₁-C₆ alkyl)₂. In other embodiments, R^{6a} is —NHC(O)NH₂. In other embodiments, R^{6a} is —NHC(O)NH(C₁-C₆ alkyl). In other embodiments, R^{6a} is —C(NH)NH₂. In other embodiments, R^{6a} is —SO₂NH₂. In other embodiments, R^{6a} is 5-10 membered heteroaryl comprising 1-3 heteroatoms selected from nitrogen, oxygen, and sulfur, wherein said heteroaryl is optionally substituted with 1-2 R^{a′}; and each R^{a′} is Cl, —OH, —CH₃, —CH₂CH₃, —CF₃, —C(O)OCH₂CH₃, or —C(O)NH₂. In some embodiments, the invention relates to a compound of formula (I-B), or a pharmaceutically acceptable salt thereof, wherein R^{6a} is H, —CN, —OCH₃, —NH(CH₃), —N(CH₃)₂, —NHC(O)NH₂, —NHC(O)NHCH₃, —OH, —OCH₂CH₂OCH₃, —OCH₂CH₂N(CH₃)₂, —C(O)NH₂, —C(O)NH(CH₃), —C(O)N(CH₃)₂, —C(O)OH, —C(O)OCH₃, —C(NH)NH₂, —C(S)NH₂, —SO₂NH₂,

In some embodiments, the invention relates to a compound of formula (I-B), or a pharmaceutically acceptable salt thereof, wherein R^{6a} is H, —CN, —OCH₃, —NH(CH₃), —OH, —OCH₂CH₂OCH₃, —OCH₂CH₂N(CH₃)₂, —C(O)NH₂, —C(O)NH(CH₃), —C(O)N(CH₃)₂, —C(O)OH, —C(O)OCH₃, or —C(S)NH₂. In other embodiments, R^{6a} is H. In other embodiments, R^{6a} is —CN. In other embodiments, R^{6a} is —OCH₃. In other embodiments, R^{6a} is —NH(CH₃). In other embodiments, R^{6a} is —OH. In other embodiments, R^{6a} is —OCH₂CH₂OCH₃. In other embodiments, R^{6a} is —OCH₂CH₂N(CH₃)₂. In other embodiments, R^{6a} is —C(O)NH₂. In other embodiments, R^{6a} is —C(O)NH(CH₃). In other embodiments, R^{6a} is —C(O)N(CH₃)₂. In other embodiments, R^{6a} is —C(O)OH. In other embodiments, R^{6a} is —C(O)OCH₃. In other embodiments, R^{6a} is —C(S)NH₂. In other embodiments, R^{6a} is —N(CH₃)₂. In other embodiments, R^{6a} is —NHC(O)NH₂. In other embodiments, R^{6a} is —NHC(O)NHCH₃. In other embodiments, R^{6a} is —C(NH)NH₂. In other embodiments, R^{6a} is —SO₂NH₂. In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In some embodiments, the invention relates to a compound of formula (I-B), or a pharmaceutically acceptable salt thereof, wherein R^{9a} is H, halo, C₁-C₆ alkyl, C₁-C₆ alkoxy, —C(O)OH, or —C(O)O(C₁-C₆ alkyl). In some embodiments, the invention relates to a compound of formula (I-B), or a pharmaceutically acceptable salt thereof, wherein R^{9a} is H, halo, C₁-C₆ alkyl, or C₁-C₆ alkoxy. In other embodiments, R^{9a} is halo. In other embodiments, R^{9a} is C₁-C₆ alkyl. In other embodiments, R^{9a} is C₁-C₆ alkoxy. In other embodiments, R^{9a} is —C(O)OH. In other embodiments, R^{9a} is —C(O)O(C₁-C₆ alkyl). In some embodiments, the invention relates to a compound of formula (I-B), or a pharmaceutically acceptable salt thereof, wherein R^{9a} is H, Cl, Br, —CH₃, —OCH₃, —OCH₂CH₃, —C(O)OH, or —C(O)OCH₃. In some embodiments, the invention relates to a compound of formula (I-B), or a pharmaceutically acceptable salt thereof, wherein R^{9a} is H, Cl, Br, —CH₃, —OCH₃, or —OCH₂CH₃. In other embodiments, R^{9a} is H. In other embodiments, R^{9a} is Cl. In other embodiments, R^{9a} is Br. In other embodiments, R^{9a} is —CH₃. In other embodiments, R^{9a} is —OCH₃. In other embodiments, R^{9a} is —OCH₂CH₃. In other embodiments, R^{9a} is —C(O)OH. In other embodiments, R^{9a} is —C(O)OCH₃.

In some embodiments, the invention relates to a compound of formula (I-B), or a pharmaceutically acceptable salt thereof, wherein Y is

In some embodiments, the invention relates to a compound of formula (I-B), or a pharmaceutically acceptable salt thereof, wherein Y is

In some embodiments, the invention relates to a compound of formula (I-B), or a pharmaceutically acceptable salt thereof, wherein Y is

In some embodiments, the invention relates to a compound of formula (I-B), or a pharmaceutically acceptable salt thereof, wherein Y is

In some embodiments, the invention relates to a compound of formula (I-B), or a pharmaceutically acceptable salt thereof, wherein Y is

In some embodiments, the invention relates to a compound of formula (I-B), or a pharmaceutically acceptable salt thereof, wherein X_{3b} is N. In some embodiments, the invention relates to a compound of formula (I-B), or a pharmaceutically acceptable salt thereof, wherein X_{3b} is CR^{3b}. In some embodiments, the invention relates to a compound of formula (I-B), or a pharmaceutically acceptable salt thereof, wherein X_{3b} is CR^{3b} and R^{3b} is H, halo, C₁-C₆ alkyl, or C₁-C₆ haloalkyl. In some embodiments, the invention relates to a compound of formula (I-B), or a pharmaceutically acceptable salt thereof, wherein X_{3b} is CR^{3b} and R^{3b} is H, C₁-C₆ alkyl, or C₁-C₆ haloalkyl. In other embodiments, R^{3b} is C₁-C₆ alkyl. In other embodiments, R^{3b} is C₁-C₆ haloalkyl. In other embodiments, R^{3b} is halo. In some embodiments, the invention relates to a compound of formula (I-B), or a pharmaceutically acceptable salt thereof, wherein R^{3b} is H, F, —CH₃, or —CF₃. In some embodiments, the invention relates to a compound of formula (I-B), or a pharmaceutically acceptable salt thereof, wherein X_{3b} is CR^{3b} and R^{3b} is R^{3b} is H, —CH₃, or —CF₃. In other embodiments, R^{3b} is H. In other embodiments, R^{3b} is —CH₃. In other embodiments, R^{3b} is —CF₃. In other embodiments, R^{3b} is D. In other embodiments, R^{3b} is —C(CD₃)₃. In other embodiments, R^{3b} is F.

In some embodiments, the invention relates to a compound of formula (I-B), or a pharmaceutically acceptable salt thereof, wherein X₆, is N. In some embodiments, the invention relates to a compound of formula (I-B), or a pharmaceutically acceptable salt thereof, wherein X₆, is CR^{6b}. In some embodiments, the invention relates to a compound of formula (I-B), or a pharmaceutically acceptable salt thereof, wherein X_{6b} is CR^{6b} and R^{6b} is H, halo, or C₁-C₆ alkyl. In other embodiments, R^{6b} is halo. In other embodiments, R^{6b} is C₁-C₆ alkyl. In some embodiments, the invention relates to a compound of formula (I-B), or a pharmaceutically acceptable salt thereof, wherein X_{6b} is CR^{6b} and R^{6b} is H, F, or —CH₃. In other embodiments, R^{6b} is H. In other embodiments, R^{6b} is F. In other embodiments, R^{6b} is —CH₃. In other embodiments, R^{6b} is D. In other embodiments, R^{6b} is —C(CD₃)₃.

In some embodiments, the invention relates to a compound of formula (I-B), or a pharmaceutically acceptable salt thereof, wherein R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo. In other embodiments, R^{2b} is D. In other embodiments, R^{2b} is —C(CD₃)₃.

In some embodiments, the invention relates to a compound of formula (I-B), or a pharmaceutically acceptable salt thereof, wherein R^{2b} is H, halo, C₁-C₆ alkyl, C₁-C₆ alkoxy, —(C₁-C₆ alkylene)-OH, or —C(O)O(C₁-C₆ alkyl). In some embodiments, the invention relates to a compound of formula (I-B), or a pharmaceutically acceptable salt thereof, wherein R^{2b} is H, halo, C₁-C₆ alkyl, C₁-C₆ alkoxy, or —(C₁-C₆ alkylene)-OH. In other embodiments, R^{2b} is H. In other embodiments, R^{2b} is halo. In other embodiments, R^{2b} is C₁-C₆ alkyl. In other embodiments, R^{2b} is C₁-C₆ alkoxy. In other embodiments, R^{2b} is —(C₁-C₆ alkylene)-OH. In other embodiments, R^{2b} is —C(O)O(C₁-C₆ alkyl). In some embodiments, the invention relates to a compound of formula (I-B), or a pharmaceutically acceptable salt thereof, wherein R^{2b} is H, F, Cl, —CH₃, —CH₂CH₃, —OCH₃, —CH₂OH, —CH₂CH₂CH₂OH, or —C(O)OCH₃. In some embodiments, the invention relates to a compound of formula (I-B), or a pharmaceutically acceptable salt thereof, wherein R^{2b} is H, F, Cl, —CH₃, —CH₂CH₃, —OCH₃, or —CH₂CH₂CH₂OH. In other embodiments, R^{2b} is H. In other embodiments, R^{2b} is F. In other embodiments, R^{2b} is Cl. In other embodiments, R^{2b} is —CH₃. In other embodiments, R^{2b} is —CH₂CH₃. In other embodiments, R^{2b} is —OCH₃. In other embodiments, R^{2b} is —CH₂CH₂CH₂OH. In other embodiments, R^{2b} is —CH₂OH. In other embodiments, R^{2b} is —C(O)OCH₃.

In some embodiments, the invention relates to a compound of formula (I-B), or a pharmaceutically acceptable salt thereof, wherein R^{4b} is H, halo, C₁-C₆ alkyl, C₁-C₆ alkenyl, C₁-C₆ haloalkyl, C₁-C₆ haloalkenyl, C₁-C₆ haloalkoxy, —(C₁-C₆ alkylene)-OH, —(C₁-C₆ haloalkylene)-OH, —(C₁-C₆ alkylene)-C(O)OH, —(C₁-C₆ haloalkylene)-C(O)OH, —C(O)O(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)- (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I-B), or a pharmaceutically acceptable salt thereof, wherein R^{4b} is H, halo, C₁-C₆ alkyl, C₁-C₆ haloalkyl, C₁-C₆ haloalkenyl, —(C₁-C₆ alkylene)-OH, —Si(C₁-C₆ alkyl)₃, or (C₁-C₆ haloalkyl)-(C₃-C₆ cycloalkyl)-. In other embodiments, R^{4b} is halo. In other embodiments, R^{4b} is C₁-C₆ alkyl. In other embodiments, R^{4b} is C₁-C₆ haloalkyl. In other embodiments, R^{4b} is C₁-C₆ haloalkenyl. In other embodiments, R^{4b} is —(C₁-C₆ alkylene)-OH. In other embodiments, R^{4b} is —Si(C₁-C₆ alkyl)₃. In other embodiments, R^{4b} is (C₁-C₆ haloalkyl)-(C₃-C₆ cycloalkyl)-. In other embodiments, R^{4b} is C₁-C₆ alkenyl. In other embodiments, R^{4b} is C₁-C₆ haloalkoxy. In other embodiments, R^{4b} is —(C₁-C₆ haloalkylene)-OH. In other embodiments, R^{4b} is —(C₁-C₆ alkylene)-C(O)OH. In other embodiments, R^{4b} is —(C₁-C₆ haloalkylene)-C(O)OH. In other embodiments, R^{4b} is —C(O)O(C₁-C₆ alkyl). In other embodiments, R^{4b} is C₆-C₁₀ aryl. In other embodiments, R^{4b} is C₃-C₁₀ cycloalkyl. In other embodiments, R^{4b} is (C₁-C₆ alkyl)-(C₃-C₆ cycloalkyl)-. In other embodiments, R^{4b} is 4-10 membered heterocyclyl comprising 1-3 heteroatoms selected from nitrogen and oxygen. In other embodiments, the cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I-B), or a pharmaceutically acceptable salt thereof, wherein R^{4b} is H, Cl, —CH₃, —CH(CH₃)₂, —C(CH₃)₃, —C(CH₃)₂(CH₂CH₃), —CF₃, —C(CH₃)₂(CHF₂), —C(CH₃)₂(CF₃), —CH₂C(CH₃)_{2F}, —CH═C(CH₃)₂, —C(═CH₂)(CF₃), —OCF₃, —C(CH₃)₂(CH₂OH), —C(CH₃)(CF₃)(CH₂OH), —C(CH₃)₂(C(O)OH), —C(CH₃)(CF₃)(C(O)OH), —C(O)OCH₃, —Si(CH₃)₃, phenyl, 1-methylcyclopropyl, 1-trifluoromethylcyclopropyl, cyclobutyl, 1-methylcyclobutyl, 3,3-difluoro-1-methylcyclobutyl, cyclopentyl, 1-methylcyclopentyl, 1-trifluoromethylcyclopentyl, 3,3-difluoro-1-methylcyclopentyl, 4,4-difluoro-1-methylcyclohexyl,

In some embodiments, the invention relates to a compound of formula (I-B), or a pharmaceutically acceptable salt thereof, wherein R^{4b} is H, Cl, —CH₃, —CH(CH₃)₂, —C(CH₃)₃, —C(CH₃)₂(CH₂CH₃), —CF₃, —C(CH₃)₂(CF₃), —C(═CH₂)(CF₃), —C(CH₃)₂(CH₂OH), —Si(CH₃)₃, or 1-trifluoromethylcyclopropyl. In other embodiments, R^{4b} is H. In other embodiments, R^{4b} is Cl. In other embodiments, R^{4b} is —CH₃. In other embodiments, R^{4b} is —CH(CH₃)₂. In other embodiments, R^{4b} is —C(CH₃)₃. In other embodiments, R^{4b} is —C(CH₃)₂(CH₂CH₃). In other embodiments, R^{4b} is —CF₃. In other embodiments, R^{4b} is —C(CH₃)₂(CF₃). In other embodiments, R^{4b} is —C(═CH₂)(CF₃). In other embodiments, R^{4b} is —C(CH₃)₂(CH₂OH). In other embodiments, R^{4b} is —Si(CH₃)₃. In other embodiments, R^{4b} is 1-trifluoromethylcyclopropyl. In other embodiments, R^{4b} is D. In other embodiments, R^{4b} is —C(CD₃)₃. In other embodiments, R^{4b} is-C(CH₃)₂(CHF₂). In other embodiments, R^{4b} is —CH₂C(CH₃)₂F. In other embodiments, R^{4b} is —CH═C(CH₃)₂. In other embodiments, R^{4b} is —OCF₃. In other embodiments, R^{4b} is —C(CH₃)(CF₃)(CH₂OH). In other embodiments, R^{4b} is —C(CH₃)₂(C(O)OH). In other embodiments, R^{4b} is —C(CH₃)(CF₃)(C(O)OH). In other embodiments, R^{4b} is —C(O)OCH₃. In other embodiments, R^{4b} is phenyl. In other embodiments, R^{4b} is 1-methylcyclopropyl. In other embodiments, R^{4b} is cyclobutyl. In other embodiments, R^{4b} is 1-methylcyclobutyl. In other embodiments, R^{4b} is 3,3-difluoro-1-methylcyclobutyl. In other embodiments, R^{4b} is cyclopentyl. In other embodiments, R^{4b} is 1-methylcyclopentyl. In other embodiments, R^{4b} is 1-trifluoromethylcyclopentyl. In other embodiments, R^{4b} is 3,3-difluoro-1-methylcyclopentyl. In other embodiments, R^{4b} is 4,4-difluoro-1-methylcyclohexyl. In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In some embodiments, the invention relates to a compound of formula (I-B), or a pharmaceutically acceptable salt thereof, wherein R^{5b} is H, halo, C₁-C₆ alkyl, C₁-C₆ haloalkyl, C₁-C₆ alkoxy, or C₃-C₆ cycloalkyl. In other embodiments, R^{5b} is halo. In other embodiments, R^{5b} is C₁-C₆ alkyl. In other embodiments, R^{5b} is C₁-C₆ haloalkyl. In other embodiments, R^{5b} is C₁-C₆ alkoxy. In other embodiments, R^{5b} is C₃-C₆ cycloalkyl. In some embodiments, the invention relates to a compound of formula (I-B), or a pharmaceutically acceptable salt thereof, wherein R^{5b} is H, F, Cl, —CH₃, —CH₂CH₃, —CH(CH₃)₂, —C(CH₃)₃, —CF₃, —OCH₃, or cyclopropyl. In other embodiments, R^{5b} is H. In other embodiments, R^{5b} is F. In other embodiments, R^{5b} is Cl. In other embodiments, R^{5b} is —CH₃. In other embodiments, R^{5b} is —CH₂CH₃. In other embodiments, R^{5b} is —CH(CH₃)₂. In other embodiments, R^{5b} is —C(CH₃)₃. In other embodiments, R^{5b} is —CF₃. In other embodiments, R^{5b} is —OCH₃. In other embodiments, R^{5b} is cyclopropyl. In other embodiments, R^{5b} is D. In other embodiments, R^{5b} is —C(CD₃)₃.

In some embodiments, the invention relates to a compound of formula (I-B), or a pharmaceutically acceptable salt thereof, wherein X_{2b} is CR^{2b}. In some embodiments, the invention relates to a compound of formula (I-B), or a pharmaceutically acceptable salt thereof, wherein X_{2b} is CR^{2b} and R^{2b} is H or C₁-C₆ alkyl. In other embodiments, R^{2b} is C₁-C₆ alkyl. In some embodiments, the invention relates to a compound of formula (I-B), or a pharmaceutically acceptable salt thereof, wherein X_{2b} is CR^{2b} and R^{2b} is H or —CH₃. In other embodiments, R^{2b} is H. In other embodiments, R^{2b} is —CH₃.

In some embodiments, the invention relates to a compound of formula (I-B), or a pharmaceutically acceptable salt thereof, wherein X_{5b} is N. In some embodiments, the invention relates to a compound of formula (I-B), or a pharmaceutically acceptable salt thereof, wherein X_{5b} is CR^{5b}. In some embodiments, the invention relates to a compound of formula (I-B), or a pharmaceutically acceptable salt thereof, wherein X_{5b} is CR^{5b} and R^{5b} is H or C₁-C₆ alkyl. In other embodiments, R^{5b} is C₁-C₆ alkyl. In some embodiments, the invention relates to a compound of formula (I-B), or a pharmaceutically acceptable salt thereof, wherein X_{5b} is CR^{5b} and R^{5b} is H or —CH₃. In other embodiments, R^{5b} is H. In other embodiments, R^{5b} is —CH₃.

In some embodiments, the invention relates to a compound of formula (I-B), or a pharmaceutically acceptable salt thereof, wherein X_{6b} is CR^{6b}. In some embodiments, the invention relates to a compound of formula (I-B), or a pharmaceutically acceptable salt thereof, wherein X_{6b} is CR^{6b} and R^{6b} is H, C₁-C₆ alkyl, or —N(C₁-C₆ alkyl)(C₂-C₆ alkyl). In other embodiments, R^{6b} is C₁-C₆ alkyl. In other embodiments, R^{6b} is —N(C₁-C₆ alkyl)(C₂-C₆ alkyl). In some embodiments, the invention relates to a compound of formula (I-B), or a pharmaceutically acceptable salt thereof, wherein X_{6b} is CR^{6b} and R^{6b} is R^{6b} is H, —CH₃, —N(CH₃)(CH₂CH₂CH₂CH₃), —N(CH₂CH₃)(CH₂CH₂CH₃), —N(CH₂CH₂CH₃)₂, or —N(CH₂CH(CH₃)₂)₂. In other embodiments, R^{6b} is H. In other embodiments, R^{6b} is —CH₃. In other embodiments, R^{6b} is —N(CH₃)(CH₂CH₂CH₂CH₃). In other embodiments, R^{6b} is —N(CH₂CH₃)(CH₂CH₂CH₃). In other embodiments, R^{6b} is —N(CH₂CH₂CH₃)₂. In other embodiments, R^{6b} is —N(CH₂CH(CH₃)₂)₂.

In some embodiments, the invention relates to a compound of formula (I-B), or a pharmaceutically acceptable salt thereof, wherein Z is a 5-6 membered aromatic or nonaromatic carbocycle optionally substituted with one to four R^{z}. In some embodiments, the invention relates to a compound of formula (I-B), or a pharmaceutically acceptable salt thereof, wherein Z is

In other embodiments, Z is

In other embodiments, Z is

In other embodiments, Z is

In other embodiments, Z is

In other embodiments, Z is

In other embodiments, Z is

In other embodiments, Z is

In other embodiments, Z is

In some embodiments, the invention relates to a compound of formula (I-B), or a pharmaceutically acceptable salt thereof, wherein R^{z} is C₁-C₆ alkyl. In some embodiments, the invention relates to a compound of formula (I-B), or a pharmaceutically acceptable salt thereof, wherein R^{z} is —CH₃.

In some embodiments, the invention relates to a compound of formula (I-B), or a pharmaceutically acceptable salt thereof, wherein R^{7b} is —CH₃ or —CH(CH₃)₂. In other embodiments, R^{7b} is —CH₃. In other embodiments, R^{7b} is —CH(CH₃)₂.

In some embodiments, the invention relates to a compound of formula (I-B), or a pharmaceutically acceptable salt thereof, wherein R^{9b} is —C(CH₃)₃.

In some embodiments, the invention relates to a compound of formula (I-B), or a pharmaceutically acceptable salt thereof, wherein R^{10b} is H or Cl. In other embodiments, R^{10b} is H. In other embodiments, R^{10b} is Cl.

In some embodiments, the invention relates to a compound of formula (I-C):

or a pharmaceutically acceptable salt thereof, wherein:
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —NHC(O)NH₂, —NHC(O)NH(C₁-C₆ alkyl), —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′};
R^{9a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
each R^{a′} is independently halo, —OH, C₁-C₆ alkyl, C₁-C₆ haloalkyl, —C(O)O(C₁-C₆ alkyl), or —C(O)NH₂;
Y is

X_{2b} is N or CR^{2b};
X_{3b} is N or CR^{3b};
X_{4b} is N or CR^{4b};
X_{5b} is N or CR^{5b};
X_{6b} is N or CR^{6b};
Z is a 5-7 membered aromatic or nonaromatic ring optionally containing 1-3 heteroatoms selected from nitrogen and oxygen and optionally substituted with one or more R^{z};
R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{3b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ haloalkylene)-OH, —(C₁-C₆ haloalkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′};
R^{5b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{6b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)(C₂-C₆ alkyl), —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{7b} is C₁-C₆ alkyl;
R^{9b} is C₁-C₆ alkyl;
R^{10b} is H or halo;
each R^{b′} is independently C₁-C₆ alkyl or C₁-C₆ haloalkyl; and
R^{z} is halo, C₁-C₆ alkyl, C₁-C₆ alkenyl, C₁-C₆ alkoxy, C₁-C₆ haloalkenyl, —CN, —C(O)NH₂, —C(O)NH(C₁-C₆ alkyl), —C(O)N(C₁-C₆ alkyl)₂, or —C(O)OH;
   provided that:
(i) if Y is

X_{3b} is CR^{3b}, and X_{6b}, is CR^{6b}, then no more than three of R^{2b}, R^{3b}, R^{4b}, R^{5b}, and R^{6b} are H.

In some embodiments, the invention relates to a compound of formula (I-C), or a pharmaceutically acceptable salt thereof, wherein:
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
Y is

R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo.

In some embodiments, the invention relates to a compound of formula (I-C), or a pharmaceutically acceptable salt thereof, wherein R^{6a} is H, —CN, C₁-C₄ alkoxy, —NH(C₁-C₆ alkyl), —N(C₁-C₆ alkyl)₂, —NHC(O)NH₂, —NHC(O)NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′}; and each R^{a′} is Cl, —OH, —CH₃, —CH₂CH₃, —CF₃, —C(O)OCH₂CH₃, or —C(O)NH₂. In some embodiments, the invention relates to a compound of formula (I-C), or a pharmaceutically acceptable salt thereof, wherein R^{6a} is H, —CN, C₁-C₄ alkoxy, —NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂. In other embodiments, R^{6a} is C₁-C₄ alkoxy. In other embodiments, R^{6′} is —NH(C₁-C₆ alkyl). In other embodiments, R^{6a} is —O(C₁-C₆ alkylene)-(C₁-C₆ alkoxy). In other embodiments, R^{6a} is —O(C₁-C₆ alkylene)-N(C₁-C₆ alkyl)₂. In other embodiments, R^{6a} is —C(O)NH(C₁-C₆ alkyl). In other embodiments, R^{6a} is —C(O)N(C₁-C₆ alkyl)₂. In other embodiments, R^{6a} is —C(O)O(C₁-C₆ alkyl). In other embodiments, R^{6a} is —N(C₁-C₆ alkyl)₂. In other embodiments, R^{6a} is —NHC(O)NH₂. In other embodiments, R^{6a} is —NHC(O)NH(C₁-C₆ alkyl). In other embodiments, R^{6a} is —C(NH)NH₂. In other embodiments, R^{6a} is —SO₂NH₂. In other embodiments, R^{6a} is 5-10 membered heteroaryl comprising 1-3 heteroatoms selected from nitrogen, oxygen, and sulfur, wherein said heteroaryl is optionally substituted with 1-2 R^{a′}; and each R^{a′} is Cl, —OH, —CH₃, —CH₂CH₃, —CF₃, —C(O)OCH₂CH₃, or —C(O)NH₂. In some embodiments, the invention relates to a compound of formula (I-C), or a pharmaceutically acceptable salt thereof, wherein R^{6a} is H, —CN, —OCH₃, —NH(CH₃), —N(CH₃)₂, —NHC(O)NH₂, —NHC(O)NHCH₃, —OH, —OCH₂CH₂OCH₃, —OCH₂CH₂N(CH₃)₂, —C(O)NH₂, —C(O)NH(CH₃), —C(O)N(CH₃)₂, —C(O)OH, —C(O)OCH₃, —C(NH)NH₂, —C(S)NH₂, —SO₂NH₂,

In some embodiments, the invention relates to a compound of formula (I-C), or a pharmaceutically acceptable salt thereof, wherein R^{6a} is H, —CN, —OCH₃, —NH(CH₃), —OH, —OCH₂CH₂OCH₃, —OCH₂CH₂N(CH₃)₂, —C(O)NH₂, —C(O)NH(CH₃), —C(O)N(CH₃)₂, —C(O)OH, —C(O)OCH₃, or —C(S)NH₂. In other embodiments, R^{6a} is H. In other embodiments, R^{6a} is —CN. In other embodiments, R^{6a} is —OCH₃. In other embodiments, R^{6a} is —NH(CH₃). In other embodiments, R^{6a} is —OH. In other embodiments, R^{6a} is —OCH₂CH₂OCH₃. In other embodiments, R^{6a} is —OCH₂CH₂N(CH₃)₂. In other embodiments, R^{6a} is —C(O)NH₂. In other embodiments, R^{6a} is —C(O)NH(CH₃). In other embodiments, R^{6a} is —C(O)N(CH₃)₂. In other embodiments, R^{6a} is —C(O)OH. In other embodiments, R^{6a} is —C(O)OCH₃. In other embodiments, R^{6a} is —C(S)NH₂. In other embodiments, R^{6a} is —N(CH₃)₂. In other embodiments, R^{6a} is —NHC(O)NH₂. In other embodiments, R^{6a} is —NHC(O)NHCH₃. In other embodiments, R^{6a} is —C(NH)NH₂. In other embodiments, R^{6a} is —SO₂NH₂. In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In some embodiments, the invention relates to a compound of formula (I-C), or a pharmaceutically acceptable salt thereof, wherein R^{9a} is H, halo, C₁-C₆ alkyl, C₁-C₆ alkoxy, or —C(O)O(C₁-C₆ alkyl). In some embodiments, the invention relates to a compound of formula (I-C), or a pharmaceutically acceptable salt thereof, wherein R^{9a} is H, halo, C₁-C₆ alkyl, or C₁-C₆ alkoxy. In other embodiments, R^{9a} is halo. In other embodiments, R^{9a} is C₁-C₆ alkyl. In other embodiments, R^{9a} is C₁-C₆ alkoxy. In other embodiments, R^{9a} is —C(O)O(C₁-C₆ alkyl). In some embodiments, the invention relates to a compound of formula (I-C), or a pharmaceutically acceptable salt thereof, wherein R^{9a} is H, Cl, Br, —CH₃, —OCH₃, —OCH₂CH₃, or —C(O)OCH₃. In some embodiments, the invention relates to a compound of formula (I-C), or a pharmaceutically acceptable salt thereof, wherein R^{9a} is H, Cl, Br, —CH₃, —OCH₃, or —OCH₂CH₃. In other embodiments, R^{9a} is H. In other embodiments, R^{9a} is Cl. In other embodiments, R^{9a} is Br. In other embodiments, R^{9a} is —CH₃. In other embodiments, R^{9a} is —OCH₃. In other embodiments, R^{9a} is —OCH₂CH₃. In other embodiments, R^{9a} is —C(O)OCH₃.

In some embodiments, the invention relates to a compound of formula (I-C), or a pharmaceutically acceptable salt thereof, wherein Y

In some embodiments, the invention relates to a compound of formula (I-C), or a pharmaceutically acceptable salt thereof, wherein Y is

In some embodiments, the invention relates to a compound of formula (I-C), or a pharmaceutically acceptable salt thereof, wherein

In some embodiments, the invention relates to a compound of formula (I-C), or a pharmaceutically acceptable salt thereof, wherein

In some embodiments, the invention relates to a compound of formula (I-C), or a pharmaceutically acceptable salt thereof, wherein Y is

In some embodiments, the invention relates to a compound of formula (I-C), or a pharmaceutically acceptable salt thereof, wherein X_{3b} is N. In some embodiments, the invention relates to a compound of formula (I-C), or a pharmaceutically acceptable salt thereof, wherein X_{3b} is CR^{3b} and R^{3b}. In some embodiments, the invention relates to a compound of formula (I-C), or a pharmaceutically acceptable salt thereof, wherein X_{3b} is CR^{3b} and R^{3b} is H, halo, C₁-C₆ alkyl, or C₁-C₆ haloalkyl. In some embodiments, the invention relates to a compound of formula (I-C), or a pharmaceutically acceptable salt thereof, wherein X_{3b} is CR^{3b} and R^{3b} is H, C₁-C₆ alkyl, or C₁-C₆ haloalkyl. In other embodiments, R^{3b} is C₁-C₆ alkyl. In other embodiments, R^{3b} is C₁-C₆ haloalkyl. In other embodiments, R^{3b} is halo. In some embodiments, the invention relates to a compound of formula (I-C), or a pharmaceutically acceptable salt thereof, wherein R^{3b} is H, F, —CH₃, or —CF₃. In some embodiments, the invention relates to a compound of formula (I-C), or a pharmaceutically acceptable salt thereof, wherein X_{3b} is CR^{3b} and R^{3b} is H, —CH₃, or —CF₃. In other embodiments, R^{3b} is H. In other embodiments, R^{3b} is —CH₃. In other embodiments, R^{3b} is —CF₃. In other embodiments, R^{3b} is D. In other embodiments, R^{3b} is —C(CD₃)₃. In other embodiments, R^{3b} is F.

In some embodiments, the invention relates to a compound of formula (I-C), or a pharmaceutically acceptable salt thereof, wherein X_{6b} is N. In some embodiments, the invention relates to a compound of formula (I-C), or a pharmaceutically acceptable salt thereof, wherein X_{6b} is CR^{6b}. In some embodiments, the invention relates to a compound of formula (I-C), or a pharmaceutically acceptable salt thereof, wherein X_{6b} is CR^{6b} and R^{6b} is H, halo, or C₁-C₆ alkyl. In other embodiments, R^{6b} is halo. In other embodiments, R^{6b} is C₁-C₆ alkyl. In some embodiments, the invention relates to a compound of formula (I-C), or a pharmaceutically acceptable salt thereof, wherein X_{6b} is CR^{6b} and R^{6b} is H, F, or —CH₃. In other embodiments, R^{6b} is H. In other embodiments, R^{6b} is F. In other embodiments, R^{6b} is —CH₃. In other embodiments, R^{6b} is D. In other embodiments, R^{6b} is —C(CD₃)₃.

In some embodiments, the invention relates to a compound of formula (I-C), or a pharmaceutically acceptable salt thereof, wherein R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo. In other embodiments, R^{2b} is D. In other embodiments, R^{2b} is —C(CD₃)₃.

In some embodiments, the invention relates to a compound of formula (I-C), or a pharmaceutically acceptable salt thereof, wherein R^{2b} is halo, C₁-C₆ alkyl, C₁-C₆ alkoxy, —(C₁-C₆ alkylene)-OH, or —C(O)O(C₁-C₆ alkyl). In some embodiments, the invention relates to a compound of formula (I-C), or a pharmaceutically acceptable salt thereof, wherein R^{2b} is halo, C₁-C₆ alkyl, C₁-C₆ alkoxy, or —(C₁-C₆ alkylene)-OH. In other embodiments, R^{2b} is halo. In other embodiments, R^{2b} is C₁-C₆ alkyl. In other embodiments, R^{2b} is C₁-C₆ alkoxy. In other embodiments, R^{2b} is —(C₁-C₆ alkylene)-OH. In other embodiments, R^{2b} is —C(O)O(C₁-C₆ alkyl). In some embodiments, the invention relates to a compound of formula (I-C), or a pharmaceutically acceptable salt thereof, wherein R^{2b} is F, Cl, —CH₃, —CH₂CH₃, —OCH₃, —CH₂OH, —CH₂CH₂CH₂OH, or —C(O)OCH₃. In some embodiments, the invention relates to a compound of formula (I-C), or a pharmaceutically acceptable salt thereof, wherein R^{2b} is F, Cl, —CH₃, —CH₂CH₃, —OCH₃, or —CH₂CH₂CH₂OH. In other embodiments, R^{2b} is F. In other embodiments, R^{2b} is Cl. In other embodiments, R^{2b} is —CH₃. In other embodiments, R^{2b} is —CH₂CH₃. In other embodiments, R^{2b} is —OCH₃. In other embodiments, R^{2b} is —CH₂CH₂CH₂OH. In other embodiments, R^{2b} is —CH₂OH. In other embodiments, R^{2b} is —C(O)OCH₃.

In some embodiments, the invention relates to a compound of formula (I-C), or a pharmaceutically acceptable salt thereof, wherein R^{4b} is H, halo, C₁-C₆ alkyl, C₁-C₆ alkenyl, C₁-C₆ haloalkyl, C₁-C₆ haloalkenyl, C₁-C₆ haloalkoxy, —(C₁-C₆ alkylene)-OH, —(C₁-C₆ haloalkylene)-OH, —(C₁-C₆ alkylene)-C(O)OH, —(C₁-C₆ haloalkylene)-C(O)OH, —C(O)(O)(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I-C), or a pharmaceutically acceptable salt thereof, wherein R^{4b} is H, halo, C₁-C₆ alkyl, C₁-C₆ haloalkyl, C₁-C₆ haloalkenyl, —(C₁-C₆ alkylene)-OH, —Si(C₁-C₆ alkyl)₃, or (C₁-C₆ haloalkyl)-(C₃-C₆ cycloalkyl)-. In other embodiments, R^{4b} is halo. In other embodiments, R^{4b} is C₁-C₆ alkyl. In other embodiments, R^{4b} is C₁-C₆ haloalkyl. In other embodiments, R^{4b} is C₁-C₆ haloalkenyl. In other embodiments, R^{4b} is —(C₁-C₆ alkylene)-OH. In other embodiments, R^{4b} is —Si(C₁-C₆ alkyl)₃. In other embodiments, R^{4b} is (C₁-C₆ haloalkyl)-(C₃-C₆ cycloalkyl)-. In other embodiments, R^{4b} is C₁-C₆ alkenyl. In other embodiments, R^{4b} is C₁-C₆ haloalkoxy. In other embodiments, R^{4b} is —(C₁-C₆ haloalkylene)-OH. In other embodiments, R^{4b} is —(C₁-C₆ alkylene)-C(O)OH. In other embodiments, R^{4b} is —(C₁-C₆ haloalkylene)-C(O)OH. In other embodiments, R^{4b} is —C(O)(O)(C₁-C₆ alkyl). In other embodiments, R^{4b} is C₆-C₁₀ aryl. In other embodiments, R^{4b} is C₃-C₁₀ cycloalkyl. In other embodiments, R^{4b} is (C₁-C₆ alkyl)-(C₃-C₆ cycloalkyl)-. In other embodiments, R^{4b} is 4-10 membered heterocyclyl comprising 1-3 heteroatoms selected from nitrogen and oxygen. In other embodiments, the cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I-C), or a pharmaceutically acceptable salt thereof, wherein R^{4b} is H, Cl, —CH₃, —CH(CH₃)₂, —C(CH₃)₃, —C(CH₃)₂(CH₂CH₃), —CF₃, —C(CH₃)₂(CHF₂), —C(CH₃)₂(CF₃), —CH₂C(CH₃)₂F, —CH═C(CH₃)₂, —C(═CH₂)(CF₃), —OCF₃, —C(CH₃)₂(CH₂OH), —C(CH₃)(CF₃)(CH₂OH), —C(CH₃)₂(C(O)OH), —C(CH₃)(CF₃)(C(O)OH), —C(O)OCH₃, —Si(CH₃)₃, phenyl, 1-methylcyclopropyl, 1-trifluoromethylcyclopropyl, cyclobutyl, 1-methylcyclobutyl, 3,3-difluoro-1-methylcyclobutyl, cyclopentyl, 1-methylcyclopentyl, 1-trifluoromethylcyclopentyl, 3,3-difluoro-1-methylcyclopentyl, 4,4-difluoro-1-methylcyclohexyl,

In some embodiments, the invention relates to a compound of formula (I-C), or a pharmaceutically acceptable salt thereof, wherein R^{4b} is H, Cl, —CH₃, —CH(CH₃)₂, —C(CH₃)₃, —C(CH₃)₂(CH₂CH₃), —CF₃, —C(CH₃)₂(CF₃), —C(═CH₂)(CF₃), —C(CH₃)₂(CH₂OH), —Si(CH₃)₃, or 1-trifluoromethylcyclopropyl. In other embodiments, R^{4b} is H. In other embodiments, R^{4b} is Cl. In other embodiments, R^{4b} is —CH₃. In other embodiments, R^{4b} is —CH(CH₃)₂. In other embodiments, R^{4b} is —C(CH₃)₃. In other embodiments, R^{4b} is —C(CH₃)₂(CH₂CH₃). In other embodiments, R^{4b} is —CF₃. In other embodiments, R^{4b} is —C(CH₃)₂(CF₃). In other embodiments, R^{4b} is —C(═CH₂)(CF₃). In other embodiments, R^{4b} is —C(CH₃)₂(CH₂OH). In other embodiments, R^{4b} is —Si(CH₃)₃. In other embodiments, R^{4b} is 1-trifluoromethylcyclopropyl. In other embodiments, R^{4b} is D. In other embodiments, R^{4b} is —C(CD₃)₃. In other embodiments, R^{4b} is —C(CH₃)₂(CHF₂). In other embodiments, R^{4b} is —CH₂C(CH₃)₂F. In other embodiments, R^{4b} is —CH═C(CH₃)₂. In other embodiments, R^{4b} is —OCF₃. In other embodiments, R^{4b} is —C(CH₃)(CF₃)(CH₂OH). In other embodiments, R^{4b} is —C(CH₃)₂(C(O)OH). In other embodiments, R^{4b} is —C(CH₃)(CF₃)(C(O)OH). In other embodiments, R^{4b} is —C(O)OCH₃. In other embodiments, R^{4b} is phenyl. In other embodiments, R^{4b} is 1-methylcyclopropyl. In other embodiments, R^{4b} is cyclobutyl. In other embodiments, R^{4b} is 1-methylcyclobutyl. In other embodiments, R^{4b} is 3,3-difluoro-1-methylcyclobutyl. In other embodiments, R^{4b} is cyclopentyl. In other embodiments, R^{4b} is 1-methylcyclopentyl. In other embodiments, R^{4b} is 1-trifluoromethylcyclopentyl. In other embodiments, R^{4b} is 3,3-difluoro-1-methylcyclopentyl. In other embodiments, R^{4b} is 4,4-difluoro-1-methylcyclohexyl. In other embodiments, R^{4b} is

In other embodiments, R^{4b} is

In other embodiments, R^{4b} is

In other embodiments, R^{4b} b is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In some embodiments, the invention relates to a compound of formula (I-C), or a pharmaceutically acceptable salt thereof, wherein R^{5b} is H, halo, C₁-C₆ alkyl, C₁-C₆ haloalkyl, C₁-C₆ alkoxy, or C₃-C₆ cycloalkyl. In other embodiments, R^{5b} is halo. In other embodiments, R^{5b} is C₁-C₆ alkyl. In other embodiments, R^{5b} is C₁-C₆ haloalkyl. In other embodiments, R^{5b} is C₁-C₆ alkoxy. In other embodiments, R^{5b} is or C₃-C₆ cycloalkyl. In some embodiments, the invention relates to a compound of formula (I-C), or a pharmaceutically acceptable salt thereof, wherein R^{5b} is H, F, Cl, —CH₃, —CH₂CH₃, —CH(CH₃)₂, —C(CH₃)₃, —CF₃, —OCH₃, or cyclopropyl. In other embodiments, R^{5b} is H. In other embodiments, R^{5b} is F. In other embodiments, R^{5b} is Cl. In other embodiments, R^{5b} is —CH₃. In other embodiments, R^{5b} is —CH₂CH₃. In other embodiments, R^{5b} is —CH(CH₃)₂. In other embodiments, R^{5b} is —C(CH₃)₃. In other embodiments, R^{5b} is —CF₃. In other embodiments, R^{5b} is —OCH₃. In other embodiments, R is cyclopropyl. In other embodiments, R^{5b} is D. In other embodiments, R^{5b} is —C(CD₃)₃.

In some embodiments, the invention relates to a compound of formula (I-C), or a pharmaceutically acceptable salt thereof, wherein X_{2b} is CR^{2b}. In some embodiments, the invention relates to a compound of formula (I-C), or a pharmaceutically acceptable salt thereof, wherein X_{2b} is CR^{2b} and R^{2b} is H or C₁-C₆ alkyl. In other embodiments, R^{2b} is C₁-C₆ alkyl. In some embodiments, the invention relates to a compound of formula (I-C), or a pharmaceutically acceptable salt thereof, wherein X_{2b} is CR^{2b} and R^{2b} is H or —CH₃. In other embodiments, R^{2b} is H. In other embodiments, R^{2b} is —CH₃.

In some embodiments, the invention relates to a compound of formula (I-C), or a pharmaceutically acceptable salt thereof, wherein X_{5b} is N. In some embodiments, the invention relates to a compound of formula (I-C), or a pharmaceutically acceptable salt thereof, wherein X_{5b} is CR^{5b}. In some embodiments, the invention relates to a compound of formula (I-C), or a pharmaceutically acceptable salt thereof, wherein X_{5b} is CR^{5b} and R^{5b} is H or C₁-C₆ alkyl. In other embodiments, R^{5b} is C₁-C₆ alkyl. In some embodiments, the invention relates to a compound of formula (I-C), or a pharmaceutically acceptable salt thereof, wherein X_{5b} is CR^{5b} and R^{5b} is H or —CH₃. In other embodiments, R^{5b} is H. In other embodiments, R^{5b} is —CH₃.

In some embodiments, the invention relates to a compound of formula (I-C), or a pharmaceutically acceptable salt thereof, wherein X_{6b} is CR^{6b}. In some embodiments, the invention relates to a compound of formula (I-C), or a pharmaceutically acceptable salt thereof, wherein X_{6b} is CR^{6b} and R^{6b} is H, C₁-C₆ alkyl, or —N(C₁-C₆ alkyl)(C₂-C₆ alkyl). In other embodiments, R^{6b} is C₁-C₆ alkyl. In other embodiments, R^{6b} is —N(C₁-C₆ alkyl)(C₂-C₆ alkyl). In some embodiments, the invention relates to a compound of formula (I-C), or a pharmaceutically acceptable salt thereof, wherein X_{6b} is CR^{6b} and R^{6b} is H, —CH₃, —N(CH₃)(CH₂CH₂CH₂CH₃), —N(CH₂CH₃)(CH₂CH₂CH₃), —N(CH₂CH₂CH₃)₂, or —N(CH₂CH(CH₃)₂)₂. In other embodiments, R^{6b} is H. In other embodiments, R^{6b} is —CH₃. In other embodiments, R^{6b} is —N(CH₃)(CH₂CH₂CH₂CH₃). In other embodiments, R^{6b} is —N(CH₂CH₃)(CH₂CH₂CH₃). In other embodiments, R^{6b} is —N(CH₂CH₂CH₃)₂. In other embodiments, R^{6b} is —N(CH₂CH(CH₃)₂)₂.

In some embodiments, the invention relates to a compound of formula (I-C), or a pharmaceutically acceptable salt thereof, wherein Z is a 5-6 membered aromatic or nonaromatic carbocycle optionally substituted with one to four R^{z}. In some embodiments, the invention relates to a compound of formula (I-C), or a pharmaceutically acceptable salt thereof, wherein Z is

In other embodiments Z is

In other embodiments, Z is

In other embodiments, Z is

In other embodiments, Z is

In other embodiments, Z is

In other embodiments, Z is

In other embodiments, Z is

In other embodiments, Z is

In some embodiments, the invention relates to a compound of formula (I-C), or a pharmaceutically acceptable salt thereof, wherein R^{z} is C₁-C₆ alkyl. In some embodiments, the invention relates to a compound of formula (I-C), or a pharmaceutically acceptable salt thereof, wherein R^{z} is —CH₃.

In some embodiments, the invention relates to a compound of formula (I-C), or a pharmaceutically acceptable salt thereof, wherein R^{7b} is —CH₃ or —CH(CH₃)₂. In other embodiments, R^{7b} is —CH₃. In other embodiments, R^{7b} is —CH(CH₃)₂.

In some embodiments, the invention relates to a compound of formula (I-C), or a pharmaceutically acceptable salt thereof, wherein R^{9b} is —C(CH₃)₃.

In some embodiments, the invention relates to a compound of formula (I-C), or a pharmaceutically acceptable salt thereof, wherein R^{10b} is H or Cl. In other embodiments, R^{10b} is H. In other embodiments, R^{10b} is Cl.

In some embodiments, the invention relates to a compound of formula (I-D):

or a pharmaceutically acceptable salt thereof, wherein:
R^{6a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —NHC(O)NH₂, —NHC(O)NH(C₁-C₆ alkyl), —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′};
R^{9a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
each R^{a′} is independently halo, —OH, C₁-C₆ alkyl, C₁-C₆ haloalkyl, —C(O)O(C₁-C₆ alkyl), or —C(O)NH₂;
X_{2b} is N or CR^{2b};
X_{5b} is N or CR^{5b};
X_{6b} is N or CR^{6b};
Z is a 5-7 membered aromatic or nonaromatic ring optionally containing 1-3 heteroatoms selected from nitrogen and oxygen and is optionally substituted with one or more R^{z};
R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{5b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{6b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)(C₂-C₆ alkyl), —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
R^{z} is halo, C₁-C₆ alkyl, C₁-C₆ alkenyl, C₁-C₆ alkoxy, C₁-C₆ haloalkenyl, —CN, —C(O)NH₂, —C(O)NH(C₁-C₆ alkyl), —C(O)N(C₁-C₆ alkyl)₂, or —C(O)OH.

In some embodiments, the invention relates to a compound of formula (I-D), or a pharmaceutically acceptable salt thereof, wherein:
R^{6a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂; and
R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo.

In some embodiments, the invention relates to a compound of formula (I-D), or a pharmaceutically acceptable salt thereof, wherein R^{6a} is —OH or —C(O)NH₂. In other embodiments, R^{6a} is —OH. In other embodiments, R^{6a} is —C(O)NH₂.

In some embodiments, the invention relates to a compound of formula (I-D), or a pharmaceutically acceptable salt thereof, wherein R^{9a} is H.

In some embodiments, the invention relates to a compound of formula (I-D), or a pharmaceutically acceptable salt thereof, wherein X_{2b} is CR^{2b}. In some embodiments, the invention relates to a compound of formula (I-D), or a pharmaceutically acceptable salt thereof, wherein X_{2b} is CR^{2b} and R^{2b} is H or C₁-C₆ alkyl. In other embodiments, R^{2b} is C₁-C₆ alkyl. In some embodiments, the invention relates to a compound of formula (I-D), or a pharmaceutically acceptable salt thereof, wherein X_{2b} is CR^{2b} and R^{2b} is H or —CH₃. In other embodiments, R^{2b} is H. In other embodiments, R^{2b} is —CH₃.

In some embodiments, the invention relates to a compound of formula (I-D), or a pharmaceutically acceptable salt thereof, wherein R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo. In other embodiments, R^{2b} is D. In other embodiments, R^{2b} is —C(CD₃)₃.

In some embodiments, the invention relates to a compound of formula (I-D), or a pharmaceutically acceptable salt thereof, wherein X_{5b} is N. In some embodiments, the invention relates to a compound of formula (I-D), or a pharmaceutically acceptable salt thereof, wherein X_{5b} is CR^{5b}. In some embodiments, the invention relates to a compound of formula (I-D), or a pharmaceutically acceptable salt thereof, wherein X_{5b} is CR^{5b} and R^{5b} is H or C₁-C₆ alkyl. In other embodiments, R^{5b} is C₁-C₆ alkyl. In some embodiments, the invention relates to a compound of formula (I-D), or a pharmaceutically acceptable salt thereof, wherein X₅, is CR^{5b} and R^{5b} is H or —CH₃. In other embodiments, R^{5b} is H. In other embodiments, R^{5b} is —CH₃.

In some embodiments, the invention relates to a compound of formula (I-D), or a pharmaceutically acceptable salt thereof, wherein X_{6b} is CR^{6b}. In some embodiments, the invention relates to a compound of formula (I-D), or a pharmaceutically acceptable salt thereof, wherein X_{6b} is CR^{6b} and R^{6b} is H, C₁-C₆ alkyl, or —N(C₁-C₆ alkyl)(C₂-C₆ alkyl). In other embodiments, R^{6b} is C₁-C₆ alkyl. In other embodiments, R^{6b} is —N(C₁-C₆ alkyl)(C₂-C₆ alkyl). In some embodiments, the invention relates to a compound of formula (I-D), or a pharmaceutically acceptable salt thereof, wherein X_{6b} is CR^{6b} and R^{6b} is H, —CH₃, —N(CH₃)(CH₂CH₂CH₂CH₃), —N(CH₂CH₃)(CH₂CH₂CH₃), —N(CH₂CH₂CH₃)₂, or —N(CH₂CH(CH₃)₂)₂. In other embodiments, R^{6b} is H. In other embodiments, R^{6b} is —CH₃. In other embodiments, R^{6b} is —N(CH₃)(CH₂CH₂CH₂CH₃). In other embodiments, R^{6b} is —N(CH₂CH₃)(CH₂CH₂CH₃). In other embodiments, R^{6b} is —N(CH₂CH₂CH₃)₂. In other embodiments, R^{6b} is —N(CH₂CH(CH₃)₂)₂.

In some embodiments, the invention relates to a compound of formula (I-D), or a pharmaceutically acceptable salt thereof, wherein Z is a 5-6 membered aromatic or nonaromatic carbocycle optionally substituted with one to four R^{z}. In some embodiments, the invention relates to a compound of formula (I-D), or a pharmaceutically acceptable salt thereof, wherein Z is

In other embodiments, Z is

In other embodiments, Z is

In other embodiments, Z is

In other embodiments, Z is

In other embodiments, Z is

In other embodiments, Z is

In other embodiments, Z is

In other embodiments, Z is

In some embodiments, the invention relates to a compound of formula (I-D), or a pharmaceutically acceptable salt thereof, wherein R^{z} is C₁-C₆ alkyl. In some embodiments, the invention relates to a compound of formula (I-D), or a pharmaceutically acceptable salt thereof, wherein R^{z} is —CH₃.

In some embodiments, the invention relates to a compound of formula (I-E):

or a pharmaceutically acceptable salt thereof, wherein:
X₅ₐ is N or N⁺—O⁻;
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —NHC(O)NH₂, —NHC(O)NH(C₁-C₆ alkyl), —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′};
R^{9a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
each R^{a′} is independently halo, —OH, C₁-C₆ alkyl, C₁-C₆ haloalkyl, —C(O)O(C₁-C₆ alkyl), or —C(O)NH₂;
X_{3b} is N or CR^{3b};
X_{6b} is N or CR^{6b};
R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{3b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ haloalkylene)-OH, —(C₁-C₆ haloalkylene)-C(O)OH, —C(O)(C₁-C₆ alkyl), —C(O)(C₁-C₆ haloalkyl), —C(O)(O)(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, or (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′};
R^{5b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{6b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
each R^{b′} is independently C₁-C₆ alkyl or C₁-C₆ haloalkyl,
provided that if X_{3b} is CR^{3b} and X_{6b} is CR^{6b}, then no more than three of R^{2b}, R^{3b}, R^{4b}, R^{5b}, and R^{6b} are H.

In some embodiments, the invention relates to a compound of formula (I-E), or a pharmaceutically acceptable salt thereof, wherein:
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo.

In some embodiments, the invention relates to a compound of formula (I-E), or a pharmaceutically acceptable salt thereof, wherein X₅ₐ is N. In some embodiments, the invention relates to a compound of formula (I-E), or a pharmaceutically acceptable salt thereof, wherein X₅ₐ is N⁺—O⁻.

In some embodiments, the invention relates to a compound of formula (I-E), or a pharmaceutically acceptable salt thereof, wherein R^{6a} is H, —CN, C₁-C₄ alkoxy, —NH(C₁-C₆ alkyl), —N(C₁-C₆ alkyl)₂, —NHC(O)NH₂, —NHC(O)NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′}; and each R^{a′} is Cl, —OH, —CH₃, —CH₂CH₃, —CF₃, —C(O)OCH₂CH₃, or —C(O)NH₂. In some embodiments, the invention relates to a compound of formula (I-E), or a pharmaceutically acceptable salt thereof, wherein R^{6a} is H, —CN, C₁-C₄ alkoxy, —NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂. In other embodiments, R^{6a} is C₁-C₄ alkoxy. In other embodiments, R^{6a} is —NH(C₁-C₆ alkyl). In other embodiments, R^{6a} is —O(C₁-C₆ alkylene)-(C₁-C₆ alkoxy). In other embodiments, R^{6a} is —O(C₁-C₆ alkylene)-N(C₁-C₆ alkyl)₂. In other embodiments, R^{6a} is —C(O)NH(C₁-C₆ alkyl). In other embodiments, R^{6a} is —C(O)N(C₁-C₆ alkyl)₂. In other embodiments, R^{6a} is —C(O)O(C₁-C₆ alkyl). In other embodiments, R^{6a} is —N(C₁-C₆ alkyl)₂. In other embodiments, R^{6a} is —NHC(O)NH₂. In other embodiments, R^{6a} is —NHC(O)NH(C₁-C₆ alkyl). In other embodiments, R^{6a} is —C(NH)NH₂. In other embodiments, R^{6a} is —SO₂NH₂. In other embodiments, R^{6a} is 5-10 membered heteroaryl comprising 1-3 heteroatoms selected from nitrogen, oxygen, and sulfur, wherein said heteroaryl is optionally substituted with 1-2 R^{a′}; and each R^{a′} is Cl, —OH, —CH₃, —CH₂CH₃, —CF₃, —C(O)OCH₂CH₃, or —C(O)NH₂. In some embodiments, the invention relates to a compound of formula (I-E), or a pharmaceutically acceptable salt thereof, wherein R^{6a} is H, —CN, —OCH₃, —NH(CH₃), —N(CH₃)₂, —NHC(O)NH₂, —NHC(O)NHCH₃, —OH, —OCH₂CH₂OCH₃, —OCH₂CH₂N(CH₃)₂, —C(O)NH₂, —C(O)NH(CH₃), —C(O)N(CH₃)₂, —C(O)OH, —C(O)OCH₃, —C(NH)NH₂, —C(S)NH₂, —SO₂NH₂,

In some embodiments, the invention relates to a compound of formula (I-E), or a pharmaceutically acceptable salt thereof, wherein R^{6a} is H, —CN, —OCH₃, —NH(CH₃), —OH, —OCHCH₂OCH₃, —OCHOCH₂N(CH₃)₂, —C(O)NH₂, —C(O)NH(CH₃), —C(O)N(CH₃)₂, —C(O)OH, —C(O)OCH₃, or —C(S)NH₂. In other embodiments, R^{6a} is H. In other embodiments, R^{6a} is —CN. In other embodiments, R^{6a} is —OCH₃. In other embodiments, R^{6a} is —NH(CH₃). In other embodiments, R^{6a} is —OH. In other embodiments, R^{6a} is —OCH₂CH₂OCH₃. In other embodiments, R^{6a} is —OCH₂CH₂N(CH₃)₂. In other embodiments, R^{6a} is —C(O)NH₂. In other embodiments, R^{6a} is —C(O)NH(CH₃). In other embodiments, R^{6a} is —C(O)N(CH₃)₂. In other embodiments, R^{6a} is —C(O)OH. In other embodiments, R^{6a} is —C(O)OCH₃. In other embodiments, R^{6a} is —C(S)NH₂. In other embodiments, R^{6a} is —N(CH₃)₂. In other embodiments, R^{6a} is —NHC(O)NH₂. In other embodiments, R^{6a} is —NHC(O)NHCH₃. In other embodiments, R^{6a} is —C(NH)NH₂. In other embodiments, R^{6a} is —SO₂NH₂. In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} a is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is or

In some embodiments, the invention relates to a compound of formula (I-E), or a pharmaceutically acceptable salt thereof, wherein R^{9a} is H, halo, C₁-C₆ alkyl, C₁-C₆ alkoxy, or —C(O)O(C₁-C₆ alkyl). In some embodiments, the invention relates to a compound of formula (I-E), or a pharmaceutically acceptable salt thereof, wherein R^{9a} is H, halo, C₁-C₆ alkyl, or C₁-C₆ alkoxy. In other embodiments, R^{9a} is halo. In other embodiments, R^{9a} is C₁-C₆ alkyl. In other embodiments, R^{9a} is C₁-C₆ alkoxy. In other embodiments, R^{9a} is —C(O)O(C₁-C₆ alkyl). In some embodiments, the invention relates to a compound of formula (I-E), or a pharmaceutically acceptable salt thereof, wherein H, Cl, Br, —CH₃, —OCH₃, —OCH₂CH₃, or —C(O)OCH₃. In some embodiments, the invention relates to a compound of formula (I-E), or a pharmaceutically acceptable salt thereof, wherein R^{9a} is H, Cl, Br, —CH₃, —OCH₃, or —OCH₂CH₃. In other embodiments, R^{9a} is H. In other embodiments, R^{9a} is Cl. In other embodiments, R^{9a} is Br. In other embodiments, R^{9a} is —CH₃. In other embodiments, R^{9a} is —OCH₃. In other embodiments, R^{9a} is —OCH₂CH₃. In other embodiments, R^{9a} is —C(O)OCH₃.

In some embodiments, the invention relates to a compound of formula (I-E), or a pharmaceutically acceptable salt thereof, wherein X_{3b} is N. In some embodiments, the invention relates to a compound of formula (I-E), or a pharmaceutically acceptable salt thereof, wherein X_{3b} is CR^{3b}. In some embodiments, the invention relates to a compound of formula (I-E), or a pharmaceutically acceptable salt thereof, wherein X_{3b} is CR^{3b} and R^{3b} is H, halo, C₁-C₆ alkyl, or C₁-C₆ haloalkyl. In some embodiments, the invention relates to a compound of formula (I-E), or a pharmaceutically acceptable salt thereof, wherein X_{3b} is CR^{3b} and R^{3b} is H, C₁-C₆ alkyl, or C₁-C₆ haloalkyl. In other embodiments, R^{3b} is C₁-C₆ alkyl. In other embodiments, R^{3b} is C₁-C₆ haloalkyl. In other embodiments, R^{3b} is halo. In some embodiments, the invention relates to a compound of formula (I-E), or a pharmaceutically acceptable salt thereof, wherein R^{3b} is H, F, —CH₃, or —CF₃. In some embodiments, the invention relates to a compound of formula (I-E), or a pharmaceutically acceptable salt thereof, wherein X_{3b} is CR^{3b} and R^{3b} is H, —CH₃, or —CF₃. In other embodiments, R^{3b} is H. In other embodiments, R^{3b} is —CH₃. In other embodiments, R^{3b} is —CF₃. In other embodiments, R^{3b} is F.

In some embodiments, the invention relates to a compound of formula (I-E), or a pharmaceutically acceptable salt thereof, wherein X_{6b} is N. In some embodiments, the invention relates to a compound of formula (I-E), or a pharmaceutically acceptable salt thereof, wherein X_{6b} is CR^{6b}. In some embodiments, the invention relates to a compound of formula (I-E), or a pharmaceutically acceptable salt thereof, wherein X_{6b} is CR^{6b} and R^{6b} is H, halo, or C₁-C₆ alkyl. In other embodiments, R^{6b} is halo. In other embodiments, R^{6b} is C₁-C₆ alkyl. In some embodiments, the invention relates to a compound of formula (I-E), or a pharmaceutically acceptable salt thereof, wherein X_{6b} is CR^{6b} and R^{6b} is H, F, or —CH₃. In other embodiments, R^{6b} is H. In other embodiments, R^{6b} is F. In other embodiments, R^{6b} is —CH₃.

In some embodiments, the invention relates to a compound of formula (I-E), or a pharmaceutically acceptable salt thereof, wherein R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo. In other embodiments, R^{2b} is D. In other embodiments, R^{2b} is —C(CD₃)₃.

In some embodiments, the invention relates to a compound of formula (I-E), or a pharmaceutically acceptable salt thereof, wherein R_{2b} is halo, C₁-C₆ alkyl, C₁-C₆ alkoxy, —(C₁-C₆ alkylene)-OH, or —C(O)O(C₁-C₆ alkyl). In some embodiments, the invention relates to a compound of formula (I-E), or a pharmaceutically acceptable salt thereof, wherein R^{2b} is halo, C₁-C₆ alkyl, C₁-C₆ alkoxy, or —(C₁-C₆ alkylene)-OH. In other embodiments, R^{2b} is halo. In other embodiments, R^{2b} is C₁-C₆ alkyl. In other embodiments, R^{2b} is C₁-C₆ alkoxy. In other embodiments, R^{2b} is —(C₁-C₆ alkylene)-OH. In other embodiments, R^{2b} is —C(O)O(C₁-C₆ alkyl). In some embodiments, the invention relates to a compound of formula (I-E), or a pharmaceutically acceptable salt thereof, wherein R^{2b} is F, Cl, —CH₃, —CH₂CH₃, —OCH₃, —CH₂OH, —CH₂CH₂CH₂OH, or —C(O)OCH₃. In some embodiments, the invention relates to a compound of formula (I-E), or a pharmaceutically acceptable salt thereof, wherein R^{2b} is F, Cl, —CH₃, —CH₂CH₃, —OCH₃, or —CH₂CH₂CH₂OH. In other embodiments, R^{2b} is F. In other embodiments, R^{2b} is Cl. In other embodiments, R^{2b} is —CH₃. In other embodiments, R^{2b} is —CH₂CH₃. In other embodiments, R^{2b} is —OCH₃. In other embodiments, R^{2b} is —CH₂CH₂CH₂OH. In other embodiments, R^{2b} is —CH₂OH. In other embodiments, R^{2b} is —C(O)OCH₃.

In some embodiments, the invention relates to a compound of formula (I-E), or a pharmaceutically acceptable salt thereof, wherein R^{4b} is H, halo, C₁-C₆ alkyl, C₁-C₆ alkenyl, C₁-C₆ haloalkyl, C₁-C₆ haloalkenyl, C₁-C₆ haloalkoxy, —(C₁-C₆ alkylene)-OH, —(C₁-C₆ haloalkylene)-OH, —(C₁-C₆ alkylene)-C(O)OH, —(C₁-C₆ haloalkylene)-C(O)OH, —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I-E), or a pharmaceutically acceptable salt thereof, wherein R^{4b} is H, halo, C₁-C₆ alkyl, C₁-C₆ haloalkyl, C₁-C₆ haloalkenyl, —(C₁-C₆ alkylene)-OH, —Si(C₁-C₆ alkyl)₃, or (C₁-C₆ haloalkyl)-(C₃-C₆ cycloalkyl)-. In other embodiments, R^{4b} is halo. In other embodiments, R^{4b} is C₁-C₆ alkyl. In other embodiments, R^{4b} is C₁-C₆ haloalkyl. In other embodiments, R^{4b} is C₁-C₆ haloalkenyl. In other embodiments, R^{4b} is —(C₁-C₆ alkylene)-OH. In other embodiments, R^{4b} is —Si(C₁-C₆ alkyl)₃. In other embodiments, R^{4b} is (C₁-C₆ haloalkyl)-(C₃-C₆ cycloalkyl)-. In other embodiments, R^{4b} is C₁-C₆ alkenyl. In other embodiments, R^{4b} is C₁-C₆ haloalkoxy. In other embodiments, R^{4b} is —(C₁-C₆ haloalkylene)-OH. In other embodiments, R^{4b} is —(C₁-C₆ alkylene)-C(O)OH. In other embodiments, R^{4b} is —(C₁-C₆ haloalkylene)-C(O)OH. In other embodiments, R^{4b} is C₆-C₁₀ aryl. In other embodiments, R^{4b} is C₃-C₁₀ cycloalkyl. In other embodiments, R^{4b} is (C₁-C₆ alkyl)-(C₃-C₆ cycloalkyl)-. In other embodiments, R^{4b} is 4-10 membered heterocyclyl comprising 1-3 heteroatoms selected from nitrogen and oxygen. In other embodiments, the cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I-E), or a pharmaceutically acceptable salt thereof, wherein R^{4b} is H, Cl, —CH₃, —CH(CH₃)₂, —C(CH₃)₃, —C(CH₃)₂(CH₂CH₃), —CH═C(CH₃)₂, —CF₃, —C(CH₃)₂(CHF₂), —C(CH₃)₂(CF₃), —CH₂C(CH₃)₂F, —C(═CH₂)(CF₃), —OCF₃, —C(CH₃)₂(CH₂OH), —C(CH₃)(CF₃)(CH₂OH), —C(CH₃)₂(C(O)OH), —C(CH₃)(CF₃)(C(O)OH), —C(O)OCH₃, —Si(CH₃)₃, phenyl, 1-methylcyclopropyl, 1-trifluoromethylcyclopropyl, cyclobutyl, 1-methylcyclobutyl, 3,3-difluoro-1-methylcyclobutyl, cyclopentyl, 1-methylcyclopentyl, 1-trifluoromethylcyclopentyl, 3,3-difluoro-1-methylcyclopentyl, 4,4-difluoro-1-methylcyclohexyl,

In some embodiments, the invention relates to a compound of formula (I-E), or a pharmaceutically acceptable salt thereof, wherein R^{4b} is H, Cl, —CH₃, —CH(CH₃)₂, —C(CH₃)₃, —C(CH₃)₂(CH₂CH₃), —CF₃, —C(CH₃)₂(CF₃), —C(═CH₂)(CF₃), —C(CH₃)₂(CH₂OH), —Si(CH₃)₃, or 1-trifluoromethylcyclopropyl. In other embodiments, R^{4b} is H. In other embodiments, R^{4b} is Cl. In other embodiments, R^{4b} is —CH₃. In other embodiments, R^{4b} is —CH(CH₃)₂. In other embodiments, R^{4b} is —C(CH₃)₃. In other embodiments, R^{4b} is —C(CH₃)₂(CH₂CH₃). In other embodiments, R^{4b} is —CF₃. In other embodiments, R^{4b} is —C(CH₃)₂(CF₃). In other embodiments, R^{4b} is —C(═CH₂)(CF₃). In other embodiments, R^{4b} is —C(CH₃)₂(CH₂OH). In other embodiments, R^{4b} is —Si(CH₃)₃. In other embodiments, R^{4b} is 1-trifluoromethylcyclopropyl. In other embodiments, R^{4b} is D. In other embodiments, R^{4b} is —C(CD₃)₃. In other embodiments, R^{4b} is —CH═C(CH₃)₂. In other embodiments, R^{4b} is —C(CH₃)₂(CHF₂). In other embodiments, R^{4b} is —CH₂C(CH₃)₂F. In other embodiments, R^{4b} is —OCF₃. In other embodiments, R^{4b} is —C(CH₃)(CF₃)(CH₂OH). In other embodiments, R^{4b} is —C(CH₃)₂(C(O)OH). In other embodiments, R^{4b} is —C(CH₃)(CF₃)(C(O)OH). In other embodiments, R^{4b} is —C(O)OCH₃. In other embodiments, R^{4b} is phenyl. In other embodiments, R^{4b} is 1-methylcyclopropyl. In other embodiments, R^{4b} is cyclobutyl. In other embodiments, R^{4b} is 1-methylcyclobutyl. In other embodiments, R^{4b} is 3,3-difluoro-1-methylcyclobutyl. In other embodiments, R^{4b} is cyclopentyl. In other embodiments, R^{4b} is 1-methylcyclopentyl. In other embodiments, R^{4b} is 1-trifluoromethylcyclopentyl. In other embodiments, R^{4b} is 3,3-difluoro-1-methylcyclopentyl. In other embodiments, R^{4b} is 4,4-difluoro-1-methylcyclohexyl. In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} b is

In some embodiments, the invention relates to a compound of formula (I-E), or a pharmaceutically acceptable salt thereof, wherein R^{5b} is H, halo, C₁-C₆ alkyl, C₁-C₆ haloalkyl, C₁-C₆ alkoxy, or C₃-C₆ cycloalkyl. In other embodiments, R^{5b} is halo. In other embodiments, R^{5b} is C₁-C₆ alkyl. In other embodiments, R^{5b} is C₁-C₆ haloalkyl. In other embodiments, R^{5b} is C₁-C₆ alkoxy. In other embodiments, R^{5b} is C₃-C₆ cycloalkyl. In some embodiments, the invention relates to a compound of formula (I-E), or a pharmaceutically acceptable salt thereof, wherein R^{5b} is H, F, Cl, —CH₃, —CH₂CH₃, —CH(CH₃)₂, —C(CH₃)₃, —CF₃, —OCH₃, or cyclopropyl. In other embodiments, R^{5b} is H. In other embodiments, R^{5b} is F. In other embodiments, R^{5b} is Cl. In other embodiments, R^{5b} is —CH₃. In other embodiments, R^{5b} is —CH₂CH₃. In other embodiments, R^{5b} is —CH(CH₃)₂. In other embodiments, R^{5b} is —C(CH₃)₃. In other embodiments, R^{5b} is —CF₃. In other embodiments, R^{5b} is —OCH₃. In other embodiments, R^{5b} is cyclopropyl. In other embodiments, R^{5b} is D. In other embodiments, R^{5b} is —C(CD₃)₃.

In some embodiments, the invention relates to a compound of formula (I-F):

or a pharmaceutically acceptable salt thereof, wherein:
X₅ₐ is N, N⁺—O⁻, or N⁺—CH₃;
R^{6a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —NHC(O)NH₂, —NHC(O)NH(C₁-C₆ alkyl), —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′};
R^{9a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
each R^{a′} is independently halo, —OH, C₁-C₆ alkyl, C₁-C₆ haloalkyl, —C(O)O(C₁-C₆ alkyl), or —C(O)NH₂;
X_{3b} is N or CH;
R^{2b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{4b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ haloalkylene)-OH, —(C₁-C₆ haloalkylene)-C(O)OH, —C(O)(C₁-C₆ alkyl), —C(O)(C₁-C₆ haloalkyl), —C(O)(O)(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′};
R^{5b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
each R^{b′} is independently C₁-C₆ alkyl or C₁-C₆ haloalkyl.

In some embodiments, the invention relates to a compound of formula (I-F), or a pharmaceutically acceptable salt thereof, wherein:
R^{6a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
R^{2b} is, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
R^{4b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo.

In some embodiments, the invention relates to a compound of formula (I-F), or a pharmaceutically acceptable salt thereof, wherein X₅ₐ is N. In some embodiments, the invention relates to a compound of formula (I-F), or a pharmaceutically acceptable salt thereof, wherein X₅ₐ is N⁺—O⁻.

In some embodiments, the invention relates to a compound of formula (I-F), or a pharmaceutically acceptable salt thereof, wherein R^{6a} is H, —CN, —N(C₁-C₆ alkyl)₂, —NHC(O)NH₂, —NHC(O)NH(C₁-C₆ alkyl), —OH, C₁-C₆ alkoxy, —C(O)NH₂, —C(O)NH(C₁-C₆ alkyl), —C(O)N(C₁-C₆ alkyl)₂, —C(O)OH, —C(NH)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′}; and each R^{a′} is Cl, —OH, —CH₃, —CH₂CH₃, —CF₃, —C(O)OCH₂CH₃, or —C(O)NH₂. In some embodiments, the invention relates to a compound of formula (I-F), or a pharmaceutically acceptable salt thereof, wherein R^{6a} is H, —CN, —OH, —C(O)NH₂, —C(O)NH(C₁-C₆ alkyl), —C(O)N(C₁-C₆ alkyl)₂, or —C(O)OH. In other embodiments, R^{6a} is —C(O)NH(C₁-C₆ alkyl). In other embodiments, R^{6a} is —C(O)N(C₁-C₆ alkyl)₂. In other embodiments, R^{6a} is —N(C₁-C₆ alkyl)₂. In other embodiments, R^{6a} is —NHC(O)NH₂. In other embodiments, R^{6a} is —NHC(O)NH(C₁-C₆ alkyl). In other embodiments, R^{6a} is C₁-C₆ alkoxy. In other embodiments, R^{6a} is —C(NH)NH₂. In other embodiments, R^{6a} is —SO₂NH₂. In other embodiments, R^{6a} is 5-10 membered heteroaryl comprising 1-3 heteroatoms selected from nitrogen, oxygen, and sulfur, wherein said heteroaryl is optionally substituted with 1-2 R^{a′}; and each R^{a′} is Cl, —OH, —CH₃, —CH₂CH₃, —CF₃, —C(O)OCH₂CH₃, or —C(O)NH₂. In some embodiments, the invention relates to a compound of formula (I-F), or a pharmaceutically acceptable salt thereof, wherein R^{6a} is H, —CN, —N(CH₃)₂, —NHC(O)NH₂, —NHC(O)NHCH₃, —OH, —OCH₃, —C(O)NH₂, —C(O)NH(CH₃), —C(O)N(CH₃)₂, —C(O)OH, —C(NH)NH₂, —SO₂NH₂,

In some embodiments, the invention relates to a compound of formula (I-F), or a pharmaceutically acceptable salt thereof, wherein R^{6a} is H, —CN, —OH, —C(O)NH₂, —C(O)NH(CH₃), —C(O)NH(CH₃)₂, or —C(O)OH. In other embodiments, R^{6a} is H. In other embodiments, R^{6a} is —CN. In other embodiments, R^{6a} is —OH. In other embodiments, R^{6a} is —C(O)NH₂. In other embodiments, R^{6a} is —C(O)NH(CH₃). In other embodiments, R^{6a} is —C(O)NH(CH₃)₂. In other embodiments, R^{6a} is —C(O)OH. In other embodiments, R^{6a} is —N(CH₃)₂. In other embodiments, R^{6a} is —NHC(O)NH₂. In other embodiments, R^{6a} is —NHC(O)NHCH₃. In other embodiments, R^{6a} is —OCH₃. In other embodiments, R^{6a} is —C(NH)NH₂. In other embodiments, R^{6a} is —SO₂NH₂. In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In other embodiments, R^{6a} is

In some embodiments, the invention relates to a compound of formula (I-F), or a pharmaceutically acceptable salt thereof, wherein R^{9a} is H or C₁-C₆ alkyl. In other embodiments, R^{9a} is C₁-C₆ alkyl. In some embodiments, the invention relates to a compound of formula (I-F), or a pharmaceutically acceptable salt thereof, wherein R^{9a} is H or —CH₃. In other embodiments, R^{9a} is H. In other embodiments, R^{9a} is —CH₃.

In some embodiments, the invention relates to a compound of formula (I-F), or a pharmaceutically acceptable salt thereof, wherein X_{3b} is N. In some embodiments, the invention relates to a compound of formula (I-F), or a pharmaceutically acceptable salt thereof, wherein X_{3b} is CH.

In some embodiments, the invention relates to a compound of formula (I-F), or a pharmaceutically acceptable salt thereof, wherein R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo. In other embodiments, R^{2b} is D. In other embodiments, R^{2b} is —C(CD₃)₃.

In some embodiments, the invention relates to a compound of formula (I-F), or a pharmaceutically acceptable salt thereof, wherein R^{2b} is C₁-C₆ alkyl, —(C₁-C₆ alkylene)-OH, or —C(O)O(C₁-C₆ alkyl). In some embodiments, the invention relates to a compound of formula (I-F), or a pharmaceutically acceptable salt thereof, wherein R^{2b} is C₁-C₆ alkyl. In other embodiments, R^{2b} is —(C₁-C₆ alkylene)-OH. In other embodiments, R^{2b} is —C(O)O(C₁-C₆ alkyl). In some embodiments, the invention relates to a compound of formula (I-F), or a pharmaceutically acceptable salt thereof, wherein R^{2b} is —CH₃, —CH₂OH, or —C(O)OCH₃. In some embodiments, the invention relates to a compound of formula (I-F), or a pharmaceutically acceptable salt thereof, wherein R^{2b} is —CH₃. In other embodiments R^{2b} is —CH₃. In other embodiments R^{2b} is —CH₂OH. In other embodiments R^{2b} is —C(O)OCH₃.

In some embodiments, the invention relates to a compound of formula (I-F), or a pharmaceutically acceptable salt thereof, wherein R^{4b} is halo, C₁-C₆ alkyl, C₁-C₆ alkenyl, C₁-C₆ haloalkyl, C₁-C₆ haloalkenyl, C₁-C₆ haloalkoxy, or —(C₁-C₆ alkylene)-OH, —(C₁-C₆ haloalkylene)-OH, —(C₁-C₆ alkylene)-C(O)OH, —(C₁-C₆ haloalkylene)-C(O)OH, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I-F), or a pharmaceutically acceptable salt thereof, wherein R^{4b} is halo, C₁-C₆ alkyl, C₁-C₆ haloalkyl, C₁-C₆ haloalkenyl, or —(C₁-C₆ alkylene)-OH. In other embodiments, R^{4b} is halo. In other embodiments, R^{4b} is C₁-C₆ alkyl. In other embodiments, R^{4b} is C₁-C₆ haloalkyl. In other embodiments, R^{4b} is C₁-C₆ haloalkenyl. In other embodiments, R^{4b} is —(C₁-C₆ alkylene)-OH. In other embodiments, R^{4b} is C₁-C₆ alkenyl. In other embodiments, R^{4b} is C₁-C₆ haloalkoxy. In other embodiments, R^{4b} is —(C₁-C₆ haloalkylene)-OH. In other embodiments, R^{4b} is —(C₁-C₆ alkylene)-C(O)OH. In other embodiments, R^{4b} is —(C₁-C₆ haloalkylene)-C(O)OH. In other embodiments, R^{4b} is C₆-C₁₀ aryl. In other embodiments, R^{4b} is C₃-C₁₀ cycloalkyl. In other embodiments, R^{4b} is (C₁-C₆ alkyl)-(C₃-C₆ cycloalkyl)-. In other embodiments, R^{4b} is (C₁-C₆ haloalkyl)-(C₃-C₆ cycloalkyl)-. In other embodiments, R^{4b} is 4-10 membered heterocyclyl comprising 1-3 heteroatoms selected from nitrogen and oxygen. In other embodiments, the cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and each R^{b′} is independently —CH₃ or —CF₃. In some embodiments, the invention relates to a compound of formula (I-F), or a pharmaceutically acceptable salt thereof, wherein R^{4b} is Cl, —CH₃, —CH(CH₃)₂, —C(CH₃)₃, —CH═C(CH₃)₂, —CF₃, —C(CH₃)₂(CHF₂), —C(CH₃)₂(CF₃), —CH₂C(CH₃)₂F, —C(═CH₂)(CF₃), —OCF₃, —C(CH₃)₂(CH₂OH), —C(CH₃)(CF₃)(CH₂OH), —C(CH₃)₂(C(O)OH), —C(CH₃)(CF₃)(C(O)OH), phenyl, 1-methylcyclopropyl, 1-trifluoromethylcyclopropyl, cyclobutyl, 1-methylcyclobutyl, 3,3-difluoro-1-methylcyclobutyl, cyclopentyl, 1-methylcyclopentyl, 1-trifluoromethylcyclopentyl, 3,3-difluoro-1-methylcyclopentyl, 4,4-difluoro-1-methylcyclohexyl,

In some embodiments, the invention relates to a compound of formula (I-F), or a pharmaceutically acceptable salt thereof, wherein R^{4b} is Cl, —CH₃, —CH(CH₃)₂, —C(CH₃)₃, —CF₃, —C(CH₃)₂(CF₃), —C(═CH₂)(CF₃), or —C(CH₃)₂(CH₂OH). In other embodiments, R^{4b} is Cl. In other embodiments, R^{4b} is —CH₃. In other embodiments, R^{4b} is —CH(CH₃)₂. In other embodiments, R^{4b} is —C(CH₃)₃. In other embodiments, R^{4b} is —CF₃. In other embodiments, R^{4b} is —C(CH₃)₂(CF₃). In other embodiments, R^{4b} is —C(═CH₂)(CF₃). In other embodiments, R^{4b} is —C(CH₃)₂(CH₂OH). In other embodiments, R^{4b} is D. In other embodiments, R^{4b} is —C(CD₃)₃. In other embodiments, R^{4b} is —CH═C(CH₃)₂. In other embodiments, R^{4b} is —C(CH₃)₂(CHF₂). In other embodiments, R^{4b} is —CH₂C(CH₃)₂F. In other embodiments, R^{4b} is —OCF₃. In other embodiments, R^{4b} is —C(CH₃)(CF₃)(CH₂OH). In other embodiments, R^{4b} is —C(CH₃)₂(C(O)OH). In other embodiments, R^{4b} is —C(CH₃)(CF₃)(C(O)OH). In other embodiments, R^{4b} is phenyl. In other embodiments, R^{4b} is 1-methylcyclopropyl. In other embodiments, R^{4b} is 1-trifluoromethylcyclopropyl. In other embodiments, R^{4b} is cyclobutyl. In other embodiments, R^{4b} is 1-methylcyclobutyl. In other embodiments, R^{4b} is 3,3-difluoro-1-methylcyclobutyl. In other embodiments, R^{4b} is cyclopentyl. In other embodiments, R^{4b} is 1-methylcyclopentyl. In other embodiments, R^{4b} is 1-trifluoromethylcyclopentyl. In other embodiments, R^{4b} is 3,3-difluoro-1-methylcyclopentyl. In other embodiments, R^{4b} is 4,4-difluoro-1-methylcyclohexyl. In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In other embodiments, R^{4b} is

In some embodiments, the invention relates to a compound of formula (I-F), or a pharmaceutically acceptable salt thereof, wherein R^{5b} is halo, C₁-C₆ alkyl, C₁-C₆ haloalkyl, C₁-C₆ alkoxy, or C₃-C₆ cycloalkyl. In other embodiments, R^{5b} is halo. In other embodiments, R^{5b} is C₁-C₆ alkyl. In other embodiments, R^{5b} is C₁-C₆ haloalkyl. In other embodiments, R^{5b} is C₁-C₆ alkoxy. In other embodiments, R^{5b} is C₃-C₆ cycloalkyl. In some embodiments, the invention relates to a compound of formula (I-F), or a pharmaceutically acceptable salt thereof, wherein R^{5b} is F, Cl, —CH₃, —CH₂CH₃, —CH(CH₃)₂, —CF₃, —OCH₃, or cyclopropyl. In other embodiments, R^{5b} is F. In other embodiments, R^{5b} is Cl. In other embodiments, R^{5b} is —CH₃. In other embodiments, R^{5b} is —CH₂CH₃. In other embodiments, R^{5b} is —CH(CH₃)₂. In other embodiments, R^{5b} is —CF₃. In other embodiments, R^{5b} is —OCH₃. In other embodiments, R^{5b} is cyclopropyl. In other embodiments, R^{5b} is D. In other embodiments, R^{5b} is —C(CD₃)₃.

In some embodiments, the invention relates to a compound of formula (I-G):

or a pharmaceutically acceptable salt thereof, wherein:
X₅ₐ is N or N⁺—O⁻;
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —NHC(O)NH₂, —NHC(O)NH(C₁-C₆ alkyl), —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′};
R^{9a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
each R^{a′} is independently halo, —OH, C₁-C₆ alkyl, C₁-C₆ haloalkyl, —C(O)O(C₁-C₆ alkyl), or —C(O)NH₂;
X_{3b} is N or CH;
R^{2b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{4b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ haloalkylene)-OH, —(C₁-C₆ alkylene)-C(O)OH, —(C₁-C₆ haloalkylene)-C(O)OH, —C(O)(C₁-C₆ alkyl), —C(O)(C₁-C₆ haloalkyl), —C(O)(O)(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and
each R^{b′} is independently C₁-C₆ alkyl or C₁-C₆ haloalkyl.

In some embodiments, the invention relates to a compound of formula (I-G), or a pharmaceutically acceptable salt thereof, wherein:
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
R^{2b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
R^{4b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo.

In some embodiments, the invention relates to a compound of formula (I-G), or a pharmaceutically acceptable salt thereof, wherein X₅ₐ is N. In some embodiments, the invention relates to a compound of formula (I-G), or a pharmaceutically acceptable salt thereof, wherein X₅ₐ is N⁺—O⁻.

In some embodiments, the invention relates to a compound of formula (I-G), or a pharmaceutically acceptable salt thereof, wherein R^{6a} is H, —CN, C₁-C₄ alkoxy, —NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)OH, —C(O)O(C₁-C₆ alkyl), or —C(S)NH₂. In other embodiments, R^{6′} is C₁-C₄ alkoxy. In other embodiments, R^{6a} is —NH(C₁-C₆ alkyl). In other embodiments, R^{6a} is —O(C₁-C₆ alkylene)-(C₁-C₆ alkoxy). In other embodiments, R^{6a} is —O(C₁-C₆ alkylene)-N(C₁-C₆ alkyl)₂. In other embodiments, R^{6a} is —C(O)O(C₁-C₆ alkyl). In some embodiments, the invention relates to a compound of formula (I-G), or a pharmaceutically acceptable salt thereof, wherein R^{6a} is H, —CN, —OCH₃, —NH(CH₃), —OH, —OCH₂CH₂OCH₃, —OCH₂CH₂N(CH₃)₂, C(O)NH₂, —C(O)OH, —C(O)OCH₃, or —C(S)NH₂. In other embodiments, R^{6a} is H. In other embodiments, R^{6a} is —CN. In other embodiments, R^{6a} is —OCH₃. In other embodiments, R^{6a} is —NH(CH₃). In other embodiments, R^{6a} is —OH. In other embodiments, R^{6a} is —OCH₂CH₂OCH₃. In other embodiments, R^{6a} is —OCH₂CH₂N(CH₃)₂. In other embodiments, R^{6a} is C(O)NH₂. In other embodiments, R^{6a} is —C(O)OH. In other embodiments, R^{6a} is —C(O)OCH₃. In other embodiments, R^{6a} is —C(S)NH₂.

In some embodiments, the invention relates to a compound of formula (I-G), or a pharmaceutically acceptable salt thereof, wherein R^{9a} is H, halo, C₁-C₆ alkoxy, or —C(O)O(C₁-C₆ alkyl). In some embodiments, the invention relates to a compound of formula (I-G), or a pharmaceutically acceptable salt thereof, wherein R^{9a} is H, halo, or C₁-C₆ alkoxy. In other embodiments, R^{9a} is halo. In other embodiments, R^{9a} is C₁-C₆ alkoxy. In other embodiments, R^{9a} is —C(O)O(C₁-C₆ alkyl). In some embodiments, the invention relates to a compound of formula (I-G), or a pharmaceutically acceptable salt thereof, wherein R^{9a} is H, Cl, Br, —OCH₃, —OCH₂CH₃, or —C(O)OCH₃. In some embodiments, the invention relates to a compound of formula (I-G), or a pharmaceutically acceptable salt thereof, wherein R^{9a} is H, Cl, Br, —OCH₃, or —OCH₂CH₃. In other embodiments, R^{9a} is H. In other embodiments, R^{9a} is Cl. In other embodiments, R^{9a} is Br. In other embodiments, R^{9a} is —OCH₃. In other embodiments, R^{9a} is —OCH₂CH₃. In other embodiments, R^{9a} is —C(O)OCH₃.

In some embodiments, the invention relates to a compound of formula (I-G), or a pharmaceutically acceptable salt thereof, wherein X_{3b} is N. In some embodiments, the invention relates to a compound of formula (I-G), or a pharmaceutically acceptable salt thereof, wherein X_{3b} is CH.

In some embodiments, the invention relates to a compound of formula (I-G), or a pharmaceutically acceptable salt thereof, wherein R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo.

In some embodiments, the invention relates to a compound of formula (I-G), or a pharmaceutically acceptable salt thereof, wherein R^{2b} is halo, C₁-C₆ alkyl, C₁-C₆ alkoxy, or —(C₁-C₆ alkylene)-OH. In other embodiments, R^{2b} is halo. In other embodiments, R^{2b} is C₁-C₆ alkyl. In other embodiments, R^{2b} is C₁-C₆ alkoxy. In other embodiments, R^{2b} is —(C₁-C₆ alkylene)-OH. In some embodiments, the invention relates to a compound of formula (I-G), or a pharmaceutically acceptable salt thereof, wherein R^{2b} is F, Cl, —CH₃, —CH₂CH₃, —OCH₃, or —OCH₂CH₂CH₂OH. In other embodiments, R^{2b} is F. In other embodiments, R^{2b} is Cl. In other embodiments, R^{2b} is —CH₃. In other embodiments, R^{2b} is —CH₂CH₃. In other embodiments, R^{2b} is —OCH₃. In other embodiments, R^{2b} is —OCH₂CH₂CH₂OH. In other embodiments, R^{2b} is D. In other embodiments, R^{2b} is —C(CD₃)₃.

In some embodiments, the invention relates to a compound of formula (I-G), or a pharmaceutically acceptable salt thereof, wherein R^{4b} is C₁-C₆ alkyl, C₁-C₆ haloalkyl, —Si(C₁-C₆ alkyl)₃, or (C₁-C₆ haloalkyl)-(C₃-C₆ cycloalkyl)-. In other embodiments R^{4b} is C₁-C₆ alkyl. In other embodiments R^{4b} is C₁-C₆ haloalkyl. In other embodiments R^{4b} is —Si(C₁-C₆ alkyl)₃. In other embodiments R^{4b} is (C₁-C₆ haloalkyl)-(C₃-C₆ cycloalkyl)-. In some embodiments, the invention relates to a compound of formula (I-G), or a pharmaceutically acceptable salt thereof, wherein R^{4b} is-C(CH₃)₃, —C(CH₃)₂(CH₂)(CH₃), —C(CH₃)₂(CF₃), —Si(CH₃)₃, or 1-trifluoromethylcyclopropyl. In other embodiments, R^{4b} is-C(CH₃)₃. In other embodiments, R^{4b} is —C(CH₃)₂(CH₂)(CH₃). In other embodiments, R^{4b} is —C(CH₃)₂(CF₃). In other embodiments, R^{4b} is, —Si(CH₃)₃. In other embodiments, R^{4b} is 1-trifluoromethylcyclopropyl. In other embodiments, R^{4b} is D. In other embodiments, R^{4b} is —C(CD₃)₃.

In some embodiments, the invention relates to a compound of formula (II), or a pharmaceutically acceptable salt thereof, wherein:
R^{6c} is H, halo, —OH, —CN, C₁-C₆ alkoxy, or C(O)NH₂; and
Y is

In some embodiments, the invention relates to a compound of formula (II), or a pharmaceutically acceptable salt thereof, wherein R^{3c} is H, halo, or C₁-C₆ alkyl. In other embodiments, R^{3c} is halo. In other embodiments, R^{3c} is C₁-C₆ alkyl. In some embodiments, the invention relates to a compound of formula (II), or a pharmaceutically acceptable salt thereof, wherein R^{3c} is H, F, or —CH₃. In other embodiments, R^{3c} is H. In other embodiments, R^{3c} is F. In other embodiments, R^{3c} is —CH₃.

In some embodiments, the invention relates to a compound of formula (II), or a pharmaceutically acceptable salt thereof, wherein R^{4c} is H, halo, —CN, or C₁-C₆ alkoxy. In other embodiments, R^{4c} is halo. In other embodiments, R^{4c} is C₁-C₆ alkoxy. In some embodiments, the invention relates to a compound of formula (II), or a pharmaceutically acceptable salt thereof, wherein R^{4c} is H, F, —CN, or —OCH₃. In other embodiments, R^{4c} is H. In other embodiments, R^{4c} is F. In other embodiments, R^{4c} is —CN. In other embodiments, R^{4c} is —OCH₃.

In some embodiments, the invention relates to a compound of formula (II), or a pharmaceutically acceptable salt thereof, wherein R^{5c} is H, halo, —CN, C₁-C₆ alkyl, C₁-C₆ haloalkyl, C₁-C₆ alkoxy, or —C(O)O(C₁-C₆ alkyl). In other embodiments, R^{5c} is halo. In other embodiments, R^{5c} is C₁-C₆ alkyl. In other embodiments, R^{5c} is C₁-C₆ haloalkyl. In other embodiments, R^{5c} is C₁-C₆ alkoxy. In other embodiments, R^{5c} is —C(O)O(C₁-C₆ alkyl). In some embodiments, the invention relates to a compound of formula (II), or a pharmaceutically acceptable salt thereof, wherein R^{5c} is H, F, —CN, —CH₃, —CF₃, —OCH₃, or —C(O)OCH₃. In other embodiments, R^{5c} is H. In other embodiments, R^{5c} is F. In other embodiments, R^{5c} is —CN. In other embodiments, R^{5c} is —CH₃. In other embodiments, R^{5c} is —CF₃. In other embodiments, R^{5c} is —OCH₃. In other embodiments, R^{5c} is —C(O)OCH₃.

In some embodiments, the invention relates to a compound of formula (II), or a pharmaceutically acceptable salt thereof, wherein R^{6c} is H, F, Cl, —OH, —CN, —OCH₃, C(O)NH₂, or

In some embodiments, the invention relates to a compound of formula (II), or a pharmaceutically acceptable salt thereof, wherein R^{6c} is H, halo, —OH, —CN, C₁-C₆ alkoxy, or C(O)NH₂. In other embodiments, R^{6c} is halo. In other embodiments, R^{6c} is C₁-C₆ alkoxy. In some embodiments, the invention relates to a compound of formula (II), or a pharmaceutically acceptable salt thereof, wherein R^{6c} is H, F, Cl, —OH, —CN, —OCH₃, or C(O)NH₂. In other embodiments, R^{6c} is H. In other embodiments, R^{6c} is F. In other embodiments, R^{6c} is Cl. In other embodiments, R^{6c} is —OH. In other embodiments, R^{6c} is —CN. In other embodiments, R^{6c} is —OCH₃. In other embodiments, R^{6c} is C(O)NH₂. In other embodiments, R^{6c} is

In some embodiments, the invention relates to a compound of formula (II), or a pharmaceutically acceptable salt thereof, wherein R^{9c} is H, C₁-C₆ alkyl, —(C₁-C₆ alkylene)-OH, —(C₁-C₆ alkylene)-O(C₁-C₆ alkyl), or —C(O)O(C₁-C₆ alkyl). In other embodiments, R^{9c} is C₁-C₆ alkyl. In other embodiments, R^{9c} is —(C₁-C₆ alkylene)-OH. In other embodiments, R^{9c} is —(C₁-C₆ alkylene)-O(C₁-C₆ alkyl). In other embodiments, R^{9c} is —C(O)O(C₁-C₆ alkyl). In some embodiments, the invention relates to a compound of formula (II), or a pharmaceutically acceptable salt thereof, wherein R^{9c} is H, —CH₃, —CH₂OH, —CH₂OCH₃, or —C(O)OCH₂CH₃. In other embodiments, R^{9c} is H. In other embodiments, R^{9c} is —CH₃. In other embodiments, R^{9c} is —CH₂OH. In other embodiments, R^{9c} is —CH₂OCH₃. In other embodiments, R^{9c} is —C(O)OCH₂CH₃.

In some embodiments, the invention relates to a compound of formula (II), or a pharmaceutically acceptable salt thereof, wherein Y is

In some embodiments, the invention relates to a compound of formula (II), or a pharmaceutically acceptable salt thereof, wherein Y is

In some embodiments, the invention relates to a compound of formula (II), or a pharmaceutically acceptable salt thereof, wherein X_{3d} is N. In some embodiments, the invention relates to a compound of formula (II), or a pharmaceutically acceptable salt thereof, wherein X₃a is CR^{3d}. In some embodiments, the invention relates to a compound of formula (II), or a pharmaceutically acceptable salt thereof, wherein X₃a is CR^{3d} and R^{3d} is H, halo, C₁-C₆ alkyl, C₁-C₆ haloalkyl, C₁-C₆ alkoxy, or C₁-C₆ haloalkoxy. In other embodiments, R^{3d} is halo. In other embodiments, R^{3d} is C₁-C₆ alkyl. In other embodiments, R^{3d} is C₁-C₆ haloalkyl. In other embodiments, R^{3d} is C₁-C₆ alkoxy. In other embodiments, R^{3d} is C₁-C₆ haloalkoxy. In some embodiments, the invention relates to a compound of formula (II), or a pharmaceutically acceptable salt thereof, wherein X₃a is CR^{3d} and R^{3d} is H, F, Cl, —CH₃, —CH₂CH₃, —CF₃, —OCH₃, —OCH₂CH₃, or —OCF₃. In other embodiments, R^{3d} is H. In other embodiments, R^{3d} is F. In other embodiments, R^{3d} is Cl. In other embodiments, R^{3d} is —CH₃. In other embodiments, R^{3d} is —CH₂CH₃. In other embodiments, R^{3d} is —CF₃. In other embodiments, R^{3d} is —OCH₃. In other embodiments, R^{3d} is —OCH₂CH₃. In other embodiments, R^{3d} is —OCF₃.

In some embodiments, the invention relates to a compound of formula (II), or a pharmaceutically acceptable salt thereof, wherein X_{3d} is CR^{3d}; and R^{2d} and R^{3d}, together with the carbon atoms to which they are attached, form a ring of formula

In other embodiments, R^{2d} and R^{3d}, together with the carbon atoms to which they are attached, form a ring of formula

In other embodiments, R^{2d} and R^{3d}, together with the carbon atoms to which they are attached, form a ring of formula

In some embodiments, the invention relates to a compound of formula (II), or a pharmaceutically acceptable salt thereof, wherein X_{3d} is CR^{3d}; and R^{3d} and R^{4d}, together with the carbon atoms to which they are attached form a ring of formula

In other embodiments, R^{3d} and R^{4d}, together with the carbon atoms to which they are attached, form a ring of formula

In other embodiments, R^{3d} and R^{4d}, together with the carbon atoms to which they are attached, form a ring of formula

In other embodiments, R^{3d} and R^{4d}, together with the carbon atoms to which they are attached, form a ring of formula

In other embodiments, R^{3d} and R^{4d}, together with the carbon atoms to which they are attached, form a ring of formula

In other embodiments, R^{3d} and R^{4d}, together with the carbon atoms to which they are attached, form a ring of formula

In other embodiments, R^{3d} and R^{4d}, together with the carbon atoms to which they are attached, form a ring of formula

In other embodiments, R^{3d} and R^{4d}, together with the carbon atoms to which they are attached, form a ring of formula

In some embodiments, the invention relates to a compound of formula (II), or a pharmaceutically acceptable salt thereof, wherein R^{2d} is H, halo, —OH, C₁-C₆ alkyl, C₁-C₆ alkoxy, C₁-C₆ haloalkoxy, or —(C₁-C₆ alkylene)-OH. In other embodiments, R^{2d} is halo. In other embodiments, R^{2d} is C₁-C₆ alkyl. In other embodiments, R^{2d} is C₁-C₆ alkoxy. In other embodiments, R^{2d} is C₁-C₆ haloalkoxy. In other embodiments, R^{2d} is —(C₁-C₆ alkylene)-OH. In some embodiments, the invention relates to a compound of formula (II), or a pharmaceutically acceptable salt thereof, wherein R^{2d} is H, F, Cl, —OH, —CH₃, —CH₂CH₃, —CH(CH₃)₂, —C(CH₃)₃, —OCH₃, —OCH₂CH₃, —OCH(CH₃)₂, —OCF₃, or —CH₂OH. In other embodiments, R^{2d} is H. In other embodiments, R^{2d} is F. In other embodiments, R^{2d} is Cl. In other embodiments, R^{2d} is —OH. In other embodiments, R^{2d} is —CH₃. In other embodiments, R^{2d} is —CH₂CH₃. In other embodiments, R^{2d} is —CH(CH₃)₂. In other embodiments, R^{2d} is —C(CH₃)₃. In other embodiments, R^{2d} is —OCH₃. In other embodiments, R^{2d} is —OCH₂CH₃. In other embodiments, R^{2d} is —OCH(CH₃)₂. In other embodiments, R^{2d} is —OCF₃. In other embodiments, R^{2d} is —CH₂OH.

In some embodiments, the invention relates to a compound of formula (II), or a pharmaceutically acceptable salt thereof, wherein R^{4d} is H, halo, C₁-C₆ alkyl, C₁-C₆ haloalkyl, C₁-C₆ haloalkenyl, C₁-C₆ alkoxy, C₁-C₆ haloalkoxy, —(C₁-C₆ alkylene)-OH, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN. In other embodiments, R^{4d} is halo. In other embodiments, R^{4d} is C₁-C₆ alkyl. In other embodiments, R^{4d} is C₁-C₆ haloalkyl. In other embodiments, R^{4d} is C₁-C₆ haloalkenyl. In other embodiments, R^{4d} is C₁-C₆ alkoxy. In other embodiments, R^{4d} is C₁-C₆ haloalkoxy. In other embodiments, R^{4d} is —(C₁-C₆ alkylene)-OH. In other embodiments, R^{4d} is —(C₁-C₆ alkylene)-(C₁-C₆ alkoxy). In other embodiments, R^{4d} is —Si(C₁-C₆ alkyl)₃. In other embodiments, R^{4d} is —C(O)O(C₁-C₆ alkyl). In other embodiments, R^{4d} is (C₁-C₆ alkyl)-(C₃-C₆ cycloalkyl)-. In other embodiments, R^{4d} is (C₁-C₆ haloalkyl)-(C₃-C₆ cycloalkyl)-. In other embodiments, R^{4d} is C₃-C₆ cycloalkyl, wherein said cycloalkyl is optionally substituted with one or more halogen or —CN. In some embodiments, the invention relates to a compound of formula (II), or a pharmaceutically acceptable salt thereof, wherein R^{4d} is H, F, Cl, —CH₃, —CH₂CH₃, —CH(CH₃)₂, —C(CH₃)₃, —CH₂C(CH₃)₃, —CF₃, —CH(CH₃)(CF₃), —C(CH₃)₂(CF₃), —C(═CH₂)(CF₃), —OCH₃, —OCH(CH₃)₂, —OC(CH₃)₃, —OCF₃, —C(CH₃)₂OH, —C(CH₃)₂(OCH₃), —Si(CH₃)₃, —C(O)OCH₂CH₃, cyclopropyl, 1-cyanocyclopropyl, 1-methylcyclopropyl, 1-trifluoromethylcyclopropyl, 1-methylcyclobutyl, or 3,3-difluorocyclobutyl. In other embodiments, R^{4d} is H. In other embodiments, R^{4d} is F. In other embodiments, R^{4d} is Cl. In other embodiments, R^{4d} is —CH₃. In other embodiments, R^{4d} is —CH₂CH₃. In other embodiments, R^{4d} is —CH(CH₃)₂. In other embodiments, R^{4d} is —C(CH₃)₃. In other embodiments, R^{4d} is —CH₂C(CH₃)₃. In other embodiments, R^{4d} is —CF₃. In other embodiments, R^{4d} is —CH(CH₃)(CF₃). In other embodiments, R^{4d} is —C(CH₃)₂(CF₃). In other embodiments, R^{4d} is —C(═CH₂)(CF₃). In other embodiments, R^{4d} is —OCH₃. In other embodiments, R^{4d} is —OCH(CH₃)₂. In other embodiments, R^{4d} is —OC(CH₃)₃. In other embodiments, R^{4d} is —OCF₃. In other embodiments, R^{4d} is —C(CH₃)₂OH. In other embodiments, R^{4d} is —C(CH₃)₂(OCH₃). In other embodiments, R^{4d} is —Si(CH₃)₃. In other embodiments, R^{4d} is —C(O)OCH₂CH₃. In other embodiments, R^{4d} is cyclopropyl. In other embodiments, R^{4d} is 1-cyanocyclopropyl. In other embodiments, R^{4d} is 1-methylcyclopropyl. In other embodiments, R^{4d} is 1-trifluoromethylcyclopropyl. In other embodiments, R^{4d} is 1-methylcyclobutyl. In other embodiments, R^{4d} is 3,3-difluorocyclobutyl.

In some embodiments, the invention relates to a compound of formula (II), or a pharmaceutically acceptable salt thereof, wherein R^{5d} is H, halo, C₁-C₆ alkyl, C₁-C₆ haloalkyl, C₁-C₆ alkoxy, or —C(O)(C₁-C₆ alkyl). In other embodiments, R^{5d} is halo. In other embodiments, R^{5d} is C₁-C₆ alkyl. In other embodiments, R^{5d} is C₁-C₆ haloalkyl. In other embodiments, R^{5d} is C₁-C₆ alkoxy. In other embodiments, R^{5d} is —C(O)(C₁-C₆ alkyl). In some embodiments, the invention relates to a compound of formula (II), or a pharmaceutically acceptable salt thereof, wherein R^{5d} is H, F, Cl, Br, —CH₃, —C(CH₃)₃, —CF₃, —OCH₃, or —C(O)CH₃. In other embodiments, R^{5d} is H. In other embodiments, R^{5d} is F. In other embodiments, R^{5d} is Cl. In other embodiments, R^{5d} is Br. In other embodiments, R^{5d} is —CH₃. In other embodiments, R^{5d} is —C(CH₃)₃. In other embodiments, R^{5d} is —CF₃. In other embodiments, R^{5d} is —OCH₃. In other embodiments, R^{5d} is —C(O)CH₃.

In some embodiments, the invention relates to a compound of formula (II), or a pharmaceutically acceptable salt thereof, wherein R^{6d} is H, halo, or C₁-C₆ alkyl. In other embodiments, R^{6d} is halo. In other embodiments, R^{6d} is C₁-C₆ alkyl. In some embodiments, the invention relates to a compound of formula (II), or a pharmaceutically acceptable salt thereof, wherein R^{6d} is H, Cl, or —CH₃. In other embodiments, R^{6d} is H. In other embodiments, R^{6d} is Cl. In other embodiments, R^{6d} is —CH₃.

In some embodiments, the invention relates to a compound of formula (II), or a pharmaceutically acceptable salt thereof, wherein R^{7d} is —CH₃.

In some embodiments, the invention relates to a compound of formula (II), or a pharmaceutically acceptable salt thereof, wherein R^{9d} is —C(CH₃)₃.

In some embodiments, the invention relates to a compound of formula (II-A):

or a pharmaceutically acceptable salt thereof, wherein:
R^{3c} is H, halo, or C₁-C₆ alkyl;
R^{4c} is H, halo, —CN, or C₁-C₆ alkoxy;
R^{5c} is H, halo, —CN, C₁-C₆ alkyl, or C₁-C₆ alkoxy;
R^{6c} is H, halo, —OH, —CN, C₁-C₆ alkoxy, C(O)NH₂, or 5-10 membered heteroaryl comprising 1-3 heteroatoms selected from nitrogen and oxygen;
R^{9c} is H, C₁-C₆ alkyl, —(C₁-C₆ alkylene)-OH, —(C₁-C₆ alkylene)-O(C₁-C₆ alkyl), or —C(O)O(C₁-C₆ alkyl);
X_{3d} is N or CR^{3d};
   R^{2d}, R^{3d}, and R^{4d} are defined as follows:
(i) R^{2d} is H, halo, —OH, C₁-C₆ alkyl, or C₁-C₆ alkoxy;
   R^{3d} is H or halo; and
   R^{4d} is H, chloro, C₁-C₆ alkyl, C₁-C₆ haloalkyl, C₁-C₆ haloalkenyl, C₁-C₆ alkoxy, C₁-C₆ haloalkoxy, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN; or
(ii) R^{2d} is H, halo, —OH, C₁-C₆ alkyl, C₁-C₆ alkoxy, C₁-C₆ haloalkoxy, or —(C₁-C₆ alkylene)-OH; and
   R^{3d} and R^{4d}, together with the carbon atoms to which they are attached, form a ring of

R^{5d} is H, halo, C₁-C₆ alkyl, or C₁-C₆ haloalkyl; and
R^{6d} is H, halo, or C₁-C₆ alkyl, provided that:
(i) if X_{3d} is N, then no more than two of R^{2d}, R^{4d}, R^{5d}, and R^{6d} are H; and
(ii) if R^{3d} and R^{4d}, together with the carbon atoms to which they are attached, form a ring of formula:

then no more than two of R^{2d}, R^{5d}, and R^{6d} are H; and
(iii) if R^{4d} is H, then R^{2d} is C₁-C₆ alkyl and R^{5d} is C₁-C₆ alkyl or C₁-C₆ haloalkyl; and
(iv) if R^{4d} is chloro, then R^{2d} is —OH or C₁-C₆ alkyl; and
(v) if R^{4d} is —CH₃, then R^{2d} is —OH, C₁-C₆ alkyl, or C₁-C₂ alkoxy; and
(vi) if R^{4d} is —C(CH₃)₃, then R^{2d} is H, chloro, —OH, C₁-C₆ alkyl, or C₁-C₆ alkoxy; and
(vii) if R^{4d} is —CF₃, then R^{2d} is C₁-C₆ alkyl or C₁-C₆ alkoxy; and
(viii) if R^{4d} is —OCH₃, then R^{2d} is halo, —OH, C₁-C₆ alkyl, or C₁-C₆ alkoxy; and R^{5d} is halo, C₁-C₆ alkyl, or C₁-C₆ haloalkyl; and
(ix) if R^{4d} is —OC(CH₃)₃, then no more than three of R², R^{3d}, R^{5d}, and R^{6d} are H;
(x) if R^{4d} is —OCF₃, then R^{2d} is halo, —OH, C₁-C₆ alkyl, or C₁-C₆ alkoxy; and
(xi) if R^{4d} is cyclopropyl, then R^{2d} is H, chloro, —OH, C₁-C₆ alkyl, or C₁-C₆ alkoxy; and R^{6d} is H, or C₁-C₆ alkyl; and
(xii) if R^{2d} is —OCH₃ and R^{4d} is —CH₃, then R^{2d} is halo, C₁-C₆ alkyl, or C₁-C₆ haloalkyl.

In some embodiments, the invention relates to a compound of formula (II-A), or a pharmaceutically acceptable salt thereof, wherein R^{6c} is H, halo, —OH, —CN, C₁-C₆ alkoxy, or C(O)NH₂.

In some embodiments, the invention relates to a compound of formula (II-A), or a pharmaceutically acceptable salt thereof, wherein R^{3c} is H, halo, or C₁-C₆ alkyl. In other embodiments, R^{3c} is halo. In other embodiments, R^{3c} is C₁-C₆ alkyl. In some embodiments, the invention relates to a compound of formula (II-A), or a pharmaceutically acceptable salt thereof, wherein R^{3c} is H, F, or —CH₃. In other embodiments, R^{3c} is H. In other embodiments, R^{3c} is F. In other embodiments, R^{3c} is —CH₃.

In some embodiments, the invention relates to a compound of formula (II-A), or a pharmaceutically acceptable salt thereof, wherein R^{4c} is H, halo, —CN, or C₁-C₆ alkoxy. In other embodiments, R^{4c} is halo. In other embodiments, R^{4c} is C₁-C₆ alkoxy. In some embodiments, the invention relates to a compound of formula (II-A), or a pharmaceutically acceptable salt thereof, wherein R^{4c} is H, F, —CN, or —OCH₃. In other embodiments, R^{4c} is H. In other embodiments, R^{4c} is F. In other embodiments, R^{4c} is —CN. In other embodiments, R^{4c} is —OCH₃.

In some embodiments, the invention relates to a compound of formula (II-A), or a pharmaceutically acceptable salt thereof, wherein R^{5c} is H, halo, —CN, C₁-C₆ alkyl, or C₁-C₆ alkoxy. In other embodiments, R^{5c} is halo. In other embodiments, R^{5c} is C₁-C₆ alkyl. In other embodiments, R^{5c} is C₁-C₆ alkoxy. In some embodiments, the invention relates to a compound of formula (II-A), or a pharmaceutically acceptable salt thereof, wherein R^{5c} is H, F, —CN, —CH₃, or —OCH₃. In other embodiments, R^{5c} is H. In other embodiments, R^{5c} is F. In other embodiments, R^{5c} is —CN. In other embodiments, R^{5c} is —CH₃. In other embodiments, R^{5c} is —OCH₃.

In some embodiments, the invention relates to a compound of formula (II-A), or a pharmaceutically acceptable salt thereof, wherein R^{6c} is H, F, Cl, —OH, —CN, —OCH₃, —C(O)NH₂, or

In some embodiments, the invention relates to a compound of formula (II-A), or a pharmaceutically acceptable salt thereof, wherein R^{6c} is H, halo, —OH, —CN, C₁-C₆ alkoxy, or C(O)NH₂. In other embodiments, R^{6c} is halo. In other embodiments, R^{6c} is C₁-C₆ alkoxy. In some embodiments, the invention relates to a compound of formula (II-A), or a pharmaceutically acceptable salt thereof, wherein R^{6c} is H, F, Cl, —OH, —CN, —OCH₃, or —C(O)NH₂. In other embodiments, R^{6c} is H. In other embodiments, R^{6c} is F. In other embodiments, R^{6c} is Cl. In other embodiments, R^{6c} is —OH. In other embodiments, R^{6c} is —CN. In other embodiments, R^{6c} is —OCH₃. In other embodiments, R^{6c} is —C(O)NH₂. In other embodiments, R^{6c} is

In some embodiments, the invention relates to a compound of formula (II-A), or a pharmaceutically acceptable salt thereof, wherein R^{9c} is H, C₁-C₆ alkyl, —(C₁-C₆alkylene)-OH, —(C₁-C₆ alkylene)-O(C₁-C₆ alkyl), or —C(O)O(C₁-C₆ alkyl). In other embodiments, R^{9c} is C₁-C₆ alkyl. In other embodiments, R^{9c} is —(C₁-C₆ alkylene)-OH. In other embodiments, R^{9c} is —(C₁-C₆ alkylene)-O(C₁-C₆ alkyl). In other embodiments, R^{9c} is —C(O)O(C₁-C₆ alkyl). In some embodiments, the invention relates to a compound of formula (II-A), or a pharmaceutically acceptable salt thereof, wherein R^{9c} is H, —CH₃, —CH₂OH, —CH₂OCH₃, or —C(O)OCH₂CH₃. In other embodiments, R^{9c} is H. In other embodiments, R^{9c} is —CH₃. In other embodiments, R^{9c} is —CH₂OH. In other embodiments, R^{9c} is —CH₂OCH₃. In other embodiments, R^{9c} is —C(O)OCH₂CH₃.

In some embodiments, the invention relates to a compound of formula (II-A), or a pharmaceutically acceptable salt thereof, wherein X_{3d} is N. In some embodiments, the invention relates to a compound of formula (II-A), or a pharmaceutically acceptable salt thereof, wherein X_{3d} is CR^{3d}. In some embodiments, the invention relates to a compound of formula (II-A), or a pharmaceutically acceptable salt thereof, wherein X_{3d} is CR^{3d} and R^{3d} is H or halo. In other embodiments, R^{3d} is halo. In some embodiments, the invention relates to a compound of formula (II-A), or a pharmaceutically acceptable salt thereof, wherein X_{3d} is CR^{3d} and R^{3d} is H or Cl. In other embodiments, R^{3d} is H. In other embodiments, R^{3d} is Cl.

In some embodiments, the invention relates to a compound of formula (II-A), or a pharmaceutically acceptable salt thereof, wherein X_{3d} is CR^{3d}; and R^{3d} and R^{4d}, together with the carbon atoms to which they are attached, form a ring of formula

In other embodiments, R^{3d} and R^{4d}, together with the carbon atoms to which they are attached, form a ring of formula

In other embodiments, R^{3d} and R^{4d}, together with the carbon atoms to which they are attached, form a ring of formula

In other embodiments, R^{3d} and R^{4d}, together with the carbon atoms to which they are attached, form a ring of formula

In other embodiments, R^{3d} and R^{4d}, together with the carbon atoms to which they are attached, form a ring of formula

In some embodiments, the invention relates to a compound of formula (II-A), or a pharmaceutically acceptable salt thereof, wherein R^{2d} is H, halo, —OH, C₁-C₆ alkyl, or C₁-C₆ alkoxy. In other embodiments, R^{2d} is halo. In other embodiments, R^{2d} is C₁-C₆ alkyl. In other embodiments, R^{2d} is C₁-C₆ alkoxy. In some embodiments, the invention relates to a compound of formula (II-A), or a pharmaceutically acceptable salt thereof, wherein R^{2d} is H, F, Cl, —OH, —CH₃, —CH₂CH₃, —CH(CH₃)₂, —OCH₃, or —OCH₂CH₃. In other embodiments, R^{2d} is H. In other embodiments, R^{2d} is F. In other embodiments, R^{2d} is Cl. In other embodiments, R^{2d} is —OH. In other embodiments, R^{2d} is —CH₃. In other embodiments, R^{2d} is —CH₂CH₃. In other embodiments, R^{2d} is —CH(CH₃)₂. In other embodiments, R^{2d} is —OCH₃. In other embodiments, R^{2d} is —OCH₂CH₃.

In some embodiments, the invention relates to a compound of formula (II-A), or a pharmaceutically acceptable salt thereof, wherein R^{4d} is H, chloro, C₁-C₆ alkyl, C₁-C₆ haloalkyl, C₁-C₆ haloalkenyl, C₁-C₆ alkoxy, C₁-C₆ haloalkoxy, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN. In other embodiments, R^{4d} is C₁-C₆ alkyl. In other embodiments, R^{4d} is C₁-C₆ haloalkyl. In other embodiments, R^{4d} is C₁-C₆ haloalkenyl. In other embodiments, R^{4d} is C₁-C₆ alkoxy. In other embodiments, R^{4d} is C₁-C₆ haloalkoxy. In other embodiments, R^{4d} is —(C₁-C₆ alkylene)-(C₁-C₆ alkoxy). In other embodiments, R^{4d} is —Si(C₁-C₆ alkyl)₃. In other embodiments, R^{4d} is —C(O)O(C₁-C₆ alkyl). In other embodiments, R^{4d} is (C₁-C₆ alkyl)-(C₃-C₆ cycloalkyl)-. In other embodiments, R^{4d} is (C₁-C₆ haloalkyl)-(C₃-C₆ cycloalkyl)-. In other embodiments, R^{4d} is or C₃-C₆ cycloalkyl wherein said cycloalkyl is optionally substituted with one or more halogen or —CN. In some embodiments, the invention relates to a compound of formula (II-A), or a pharmaceutically acceptable salt thereof, wherein R^{4d} is H, Cl, —CH₃, —CH(CH₃)₂, —C(CH₃)₃, —CH₂C(CH₃)₃, —CF₃, —CH(CH₃)(CF₃), —C(CH₃)₂(CF₃), —C(═CH₂)(CF₃), —OCH₃, —OCF₃, —C(CH₃)₂(CH₂OCH₃), —Si(CH₃)₃, —C(O)OCH₂CH₃, cyclopropyl, 1-cyanocyclopropyl, 1-methylcyclopropyl, 1-trifluoromethylcyclopropyl, 1-methylcyclobutyl, or 3,3-difluorocyclobutyl. In other embodiments, R^{4d} is H. In other embodiments, R^{4d} is Cl. In other embodiments, R^{4d} is —CH₃. In other embodiments, R^{4d} is —CH(CH₃)₂. In other embodiments, R^{4d} is —C(CH₃)₃. In other embodiments, R^{4d} is —CH₂C(CH₃)₃. In other embodiments, R^{4d} is —CF₃. In other embodiments, R^{4d} is —CH(CH₃)(CF₃). In other embodiments, R^{4d} is —C(CH₃)₂(CF₃). In other embodiments, R^{4d} is —C(═CH₂)(CF₃). In other embodiments, R^{4d} is —OCH₃. In other embodiments, R^{4d} is —OCF₃. In other embodiments, R^{4d} is —C(CH₃)₂(CH₂OCH₃). In other embodiments, R^{4d} is —Si(CH₃)₃. In other embodiments, R^{4d} is —C(O)OCH₂CH₃. In other embodiments, R^{4d} is cyclopropyl. In other embodiments, R^{4d} is 1-cyanocyclopropyl. In other embodiments, R^{4d} is 1-methylcyclopropyl. In other embodiments, R^{4d} is 1-trifluoromethylcyclopropyl. In other embodiments, R^{4d} is 1-methylcyclobutyl. In other embodiments, R^{4d} is 3,3-difluorocyclobutyl.

In some embodiments, the invention relates to a compound of formula (II-A), or a pharmaceutically acceptable salt thereof, wherein R^{5d} is H, halo, C₁-C₆ alkyl, or C₁-C₆ haloalkyl. In other embodiments, R^{5d} is halo. In other embodiments, R^{5d} is C₁-C₆ alkyl. In other embodiments, R^{5d} is C₁-C₆ haloalkyl. In some embodiments, the invention relates to a compound of formula (II-A), or a pharmaceutically acceptable salt thereof, wherein R^{5d} is H, F, Cl, Br, —CH₃, —C(CH₃)₃, or —CF₃. In other embodiments, R^{5d} is H. In other embodiments, R^{5d} is F. In other embodiments, R^{5d} is Cl. In other embodiments, R^{5d} is Br. In other embodiments, R^{5d} is —CH₃. In other embodiments, R^{5d} is —C(CH₃)₃. In other embodiments, R^{5d} is —CF₃.

In some embodiments, the invention relates to a compound of formula (II-A), or a pharmaceutically acceptable salt thereof, wherein R^{6d} is H, halo, or C₁-C₆ alkyl. In other embodiments, R^{6d} is halo. In other embodiments, R^{6d} is C₁-C₆ alkyl. In some embodiments, the invention relates to a compound of formula (II-A), or a pharmaceutically acceptable salt thereof, wherein R^{6d} is H, Cl, or —CH₃. In other embodiments, R^{6d} is H. In other embodiments, R^{6d} is Cl. In other embodiments, R^{6d} is —CH₃.

In some embodiments, the invention relates to a compound of formula (II-B):

or a pharmaceutically acceptable salt thereof, wherein:
R^{3c} is H, halo, or C₁-C₆ alkyl;
R^{4c} is H, halo, —CN, or C₁-C₆ alkoxy;
R^{5c} is H, halo, —CN, C₁-C₆ alkyl, or C₁-C₆ alkoxy;
R^{6c} is H, halo, —OH, —CN, C₁-C₆ alkoxy, C(O)NH₂, or 5-10 membered heteroaryl comprising 1-3 heteroatoms selected from nitrogen and oxygen;
R^{9c} is H, C₁-C₆ alkyl, —(C₁-C₆ alkylene)-OH, —(C₁-C₆ alkylene)-O(C₁-C₆ alkyl), or —C(O)O(C₁-C₆ alkyl);
X_{3d} is N or CH;
R^{2d} is halo, —OH, C₁-C₆ alkyl, or C₁-C₆ alkoxy;
R^{4d} is halo, C₁-C₆ alkyl, C₁-C₆ haloalkyl, C₁-C₆ haloalkenyl, C₁-C₆ alkoxy, C₁-C₆ haloalkoxy, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN; and
R^{5d} is halo, C₁-C₆ alkyl, or C₁-C₆ haloalkyl, provided that:
(i) if R^{2d} is chloro, then X₃a is N; and
(ii) if R^{4d} is —CH₃ and Rid is —CH₃, then R^{2d} is —OH, C₁-C₆ alkyl, or C₁-C₂ alkoxy.

In some embodiments, the invention relates to a compound of formula (II-B), or a pharmaceutically acceptable salt thereof, wherein R^{6c} is H, halo, —OH, —CN, C₁-C₆ alkoxy, or C(O)NH₂.

In some embodiments, the invention relates to a compound of formula (II-B), or a pharmaceutically acceptable salt thereof, wherein R^{3c} is H or halo. In other embodiments, R^{3c} is halo. In some embodiments, the invention relates to a compound of formula (II-B), or a pharmaceutically acceptable salt thereof, wherein R^{3c} is H or F. In other embodiments, R^{3c} is H. In other embodiments, R^{3c} is F.

In some embodiments, the invention relates to a compound of formula (II-B), or a pharmaceutically acceptable salt thereof, wherein R^{4c} is H, halo, —CN, or C₁-C₆ alkoxy. In other embodiments, R^{4c} is halo. In other embodiments, R^{4c} is C₁-C₆ alkoxy. In some embodiments, the invention relates to a compound of formula (II-B), or a pharmaceutically acceptable salt thereof, wherein R^{4c} is H, F, —CN, or —OCH₃. In other embodiments, R^{4c} is H. In other embodiments, R^{4c} is F. In other embodiments, R^{4c} is —CN. In other embodiments, R^{4c} is —OCH₃.

In some embodiments, the invention relates to a compound of formula (II-B), or a pharmaceutically acceptable salt thereof, wherein R^{5c} is H, halo, or C₁-C₆ alkoxy. In other embodiments, R^{5c} is halo. In other embodiments, R^{5c} is C₁-C₆ alkoxy. In some embodiments, the invention relates to a compound of formula (II-B), or a pharmaceutically acceptable salt thereof, wherein R^{5c} is H, F, or —OCH₃. In other embodiments, R^{5c} is H. In other embodiments, R^{5c} is F. In other embodiments, R^{5c} is —OCH₃.

In some embodiments, the invention relates to a compound of formula (II-B), or a pharmaceutically acceptable salt thereof, wherein R^{6c} is H, F, Cl, —OH, —CN, —OCH₃, —C(O)NH₂, or

In some embodiments, the invention relates to a compound of formula (II-B), or a pharmaceutically acceptable salt thereof, wherein R^{6c} is H, halo, —OH, —CN, C₁-C₆ alkoxy, or C(O)NH₂. In other embodiments, R^{6c} is halo. In other embodiments, R^{6c} is C₁-C₆ alkoxy. In some embodiments, the invention relates to a compound of formula (II-B), or a pharmaceutically acceptable salt thereof, wherein R^{6c} is H, F, Cl, —OH, —CN, —OCH₃, or —C(O)NH₂. In other embodiments, R^{6c} is H. In other embodiments, R^{6c} is F. In other embodiments, R^{6c} is Cl. In other embodiments, R^{6c} is —OH. In other embodiments, R^{6c} is —CN. In other embodiments, R^{6c} is —OCH₃. In other embodiments, R^{6c} is —C(O)NH₂. In other embodiments, R^{6c} is

In some embodiments, the invention relates to a compound of formula (II-B), or a pharmaceutically acceptable salt thereof, wherein R^{9c} is H.

In some embodiments, the invention relates to a compound of formula (II-B), or a pharmaceutically acceptable salt thereof, wherein X^{3d} is N. In some embodiments, the invention relates to a compound of formula (II-B), or a pharmaceutically acceptable salt thereof, wherein X^{3d} is CH.

In some embodiments, the invention relates to a compound of formula (II-B), or a pharmaceutically acceptable salt thereof, wherein R^{2d} is halo, C₁-C₆ alkyl, or C₁-C₆ alkoxy. In other embodiments, R^{2d} is halo. In other embodiments, R^{2d} is C₁-C₆ alkyl. In other embodiments, R^{2d} is C₁-C₆ alkoxy. In some embodiments, the invention relates to a compound of formula (II-B), or a pharmaceutically acceptable salt thereof, wherein R^{2d} is F, Cl, —CH₃, or —OCH₃. In other embodiments, R^{2d} is F. In other embodiments, R^{2d} is Cl. In other embodiments, R^{2d} is —CH₃. In other embodiments, R^{2d} is —OCH₃.

In some embodiments, the invention relates to a compound of formula (II-B), or a pharmaceutically acceptable salt thereof, wherein R^{4d} is halo, C₁-C₆ alkyl, C₁-C₆ haloalkyl, C₁-C₆ haloalkenyl, or C₁-C₆ alkoxy. In other embodiments, R^{4d} is halo. In other embodiments, R^{4d} is C₁-C₆ alkyl. In other embodiments, R^{4d} is C₁-C₆ haloalkyl. In other embodiments, R^{4d} is C₁-C₆ haloalkenyl. In other embodiments, R^{4d} is C₁-C₆ alkoxy. In some embodiments, the invention relates to a compound of formula (II-B), or a pharmaceutically acceptable salt thereof, wherein R^{4d} is Cl, —CH₃, —CH(CH₃)₂, —C(CH₃)₃, —CF₃, —CH(CH₃)(CF₃), —C(CH₃)₂(CF₃), —C(═CH₂)(CF₃), or —OCH₃. In other embodiments, R^{4d} is Cl. In other embodiments, R^{4d} is —CH₃. In other embodiments, R^{4d} is —CH(CH₃)₂. In other embodiments, R^{4d} is —C(CH₃)₃. In other embodiments, R^{4d} is —CF₃. In other embodiments, R^{4d} is —CH(CH₃)(CF₃). In other embodiments, R^{4d} is —C(CH₃)₂(CF₃). In other embodiments, R^{4d} is —C(═CH₂)(CF₃). In other embodiments, R^{4d} is —OCH₃.

In some embodiments, the invention relates to a compound of formula (II-B), or a pharmaceutically acceptable salt thereof, wherein R^{5d} is halo or C₁-C₆ alkyl. In other embodiments, R^{5d} is halo. In other embodiments, R^{5d} is C₁-C₆ alkyl. In some embodiments, the invention relates to a compound of formula (II-B), or a pharmaceutically acceptable salt thereof, wherein R^{5d} is F, Cl, Br, or —CH₃. In other embodiments, R^{5d} is F. In other embodiments, R^{5d} is Cl. In other embodiments, R^{5d} is Br. In other embodiments, R^{5d} is —CH₃.

In some embodiments, the invention relates to a compound of formula (II-C):

or a pharmaceutically acceptable salt thereof, wherein:
R^{3c} is H, halo, or C₁-C₆ alkyl;
R^{4c} is H, halo, —CN, or C₁-C₆ alkoxy;
R^{5c} is H, halo, —CN, C₁-C₆ alkyl, or C₁-C₆ alkoxy;
R^{6c} is H, halo, —OH, —CN, C₁-C₆ alkoxy, C(O)NH₂, or 5-10 membered heteroaryl comprising 1-3 heteroatoms selected from nitrogen and oxygen;
R^{9c} is H, C₁-C₆ alkyl, —(C₁-C₆ alkylene)-OH, —(C₁-C₆ alkylene)-O(C₁-C₆ alkyl), or —C(O)O(C₁-C₆ alkyl);
X_{3d} is N or CH;
R^{2d} is —OH, C₁-C₆ alkyl, or C₁-C₆ alkoxy; and
R^{4d} is halo, C₁-C₆ alkyl, C₁-C₆ haloalkyl, C₁-C₆ haloalkenyl, C₁-C₆ alkoxy, C₁-C₆ haloalkoxy, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN; provided that:
(i) if R^{2d} is —OH, then R^{4d} is halo, C₁-C₆ alkyl, C₁-C₆ haloalkenyl, C₁-C₆ alkoxy, C₁-C₆ haloalkoxy, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN; and
(ii) if R^{2d} is —CH₃, then R^{4d} is halo, C₁-C₆ alkyl, C₁-C₆ haloalkyl, C₁-C₆ haloalkenyl, C₁-C₆ haloalkoxy, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN; and
(iii) if R^{2d} is —OCH₃, then R^{4d} is C₂-C₆ alkyl, C₁-C₆ haloalkyl, C₁-C₆ haloalkenyl, C₁-C₆ alkoxy, C₁-C₆ haloalkoxy, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN.

In some embodiments, the invention relates to a compound of formula (II-C), or a pharmaceutically acceptable salt thereof, wherein R^{6c} is H, halo, —OH, —CN, C₁-C₆ alkoxy, or C(O)NH₂.

In some embodiments, the invention relates to a compound of formula (II-C), or a pharmaceutically acceptable salt thereof, wherein R^{3c} is H, halo, or C₁-C₆ alkyl. In other embodiments, R^{3c} is halo. In other embodiments, R^{3c} is or C₁-C₆ alkyl. In some embodiments, the invention relates to a compound of formula (II-C), or a pharmaceutically acceptable salt thereof, wherein R^{3c} is H, F, or —CH₃. In other embodiments, R^{3c} is H. In other embodiments, R^{3c} is F. In other embodiments, R^{3c} is —CH₃.

In some embodiments, the invention relates to a compound of formula (II-C), or a pharmaceutically acceptable salt thereof, wherein R^{4c} is H, halo, —CN, or C₁-C₆ alkoxy. In other embodiments, R^{4c} is halo. In other embodiments, R^{4c} is C₁-C₆ alkoxy. In some embodiments, the invention relates to a compound of formula (II-C), or a pharmaceutically acceptable salt thereof, wherein R^{4c} is H, F, —CN, or —OCH₃. In other embodiments, R^{4c} is H. In other embodiments, R^{4c} is F. In other embodiments, R^{4c} is —CN. In other embodiments, R^{4c} is —OCH₃.

In some embodiments, the invention relates to a compound of formula (II-C), or a pharmaceutically acceptable salt thereof, wherein R^{5c} is H, halo, —CN, C₁-C₆ alkyl, or C₁-C₆ alkoxy. In other embodiments, R^{5c} is halo. In other embodiments, R^{5c} is C₁-C₆ alkyl. In other embodiments, R^{5c} is C₁-C₆ alkoxy. In some embodiments, the invention relates to a compound of formula (II-C), or a pharmaceutically acceptable salt thereof, wherein R^{5c} is H, F, —CN, —CH₃, or —OCH₃. In other embodiments, R^{5c} is H. In other embodiments, R^{5c} is F. In other embodiments, R^{5c} is —CN. In other embodiments, R^{5c} is —CH₃. In other embodiments, R^{5c} is —OCH₃.

In some embodiments, the invention relates to a compound of formula (II-C), or a pharmaceutically acceptable salt thereof, wherein R^{6c} is H, halo, —OH, —CN, C₁-C₆ alkoxy, or C(O)NH₂. In other embodiments, R^{6c} is halo. In other embodiments, R^{6c} is C₁-C₆ alkoxy. In some embodiments, the invention relates to a compound of formula (II-C), or a pharmaceutically acceptable salt thereof, wherein R^{6c} is H, F, —OH, —CN, —OCH₃, or —C(O)NH₂. In other embodiments, R^{6c} is H. In other embodiments, R^{6c} is F. In other embodiments, R^{6c} is —OH. In other embodiments, R^{6c} is —CN. In other embodiments, R^{6c} is —OCH₃. In other embodiments, R^{6c} is —C(O)NH₂.

In some embodiments, the invention relates to a compound of formula (II-C), or a pharmaceutically acceptable salt thereof, wherein R^{9c} is H, C₁-C₆ alkyl, —(C₁-C₆ alkylene)-OH, —(C₁-C₆ alkylene)-O(C₁-C₆ alkyl), or —C(O)O(C₁-C₆ alkyl). In other embodiments, R^{9c} is H. In other embodiments, R^{9c} is C₁-C₆ alkyl. In other embodiments, R^{9c} is —(C₁-C₆ alkylene)-OH. In other embodiments, R^{9c} is —(C₁-C₆ alkylene)-O(C₁-C₆ alkyl). In other embodiments, R^{9c} is —C(O)O(C₁-C₆ alkyl). In some embodiments, the invention relates to a compound of formula (II-C), or a pharmaceutically acceptable salt thereof, wherein R^{9c} is H, —CH₃, —CH₂OH, —CH₂OCH₃, or —C(O)OCH₂CH₃. In other embodiments, R^{9c} is H. In other embodiments, R^{9c} is —CH₃. In other embodiments, R^{9c} is —CH₂OH. In other embodiments, R^{9c} is —CH₂OCH₃. In other embodiments, R^{9c} is —C(O)OCH₂CH₃.

In some embodiments, the invention relates to a compound of formula (II-C), or a pharmaceutically acceptable salt thereof, wherein X_{3d} is N. In some embodiments, the invention relates to a compound of formula (II-C), or a pharmaceutically acceptable salt thereof, wherein X_{3d} is CH.

In some embodiments, the invention relates to a compound of formula (II-C), or a pharmaceutically acceptable salt thereof, wherein R^{2d} is —OH, C₁-C₆ alkyl, or C₁-C₆ alkoxy. In other embodiments, R^{2d} is C₁-C₆ alkyl. In other embodiments, R^{2d} is C₁-C₆ alkoxy. In some embodiments, the invention relates to a compound of formula (II-C), or a pharmaceutically acceptable salt thereof, wherein R^{2d} is —OH, —CH₃, —CH₂CH₃, —CH(CH₃)₂, —OCH₃, or —OCH₂CH₃. In other embodiments, R^{2d} is —OH. In other embodiments, R^{2d} is —CH₃. In other embodiments, R^{2d} is —CH₂CH₃. In other embodiments, R^{2d} is —CH(CH₃)₂. In other embodiments, R^{2d} is —OCH₃. In other embodiments, R^{2d} is —OCH₂CH₃.

In some embodiments, the invention relates to a compound of formula (II-C), or a pharmaceutically acceptable salt thereof, wherein R^{4d} is halo, C₁-C₆ alkyl, C₁-C₆ haloalkyl, C₁-C₆ haloalkoxy, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN. In other embodiments, R^{4d} is halo. In other embodiments, R^{4d} is C₁-C₆ alkyl. In other embodiments, R^{4d} is C₁-C₆ haloalkyl. In other embodiments, R^{4d} is C₁-C₆ haloalkoxy. In other embodiments, R^{4d} is —(C₁-C₆ alkylene)-(C₁-C₆ alkoxy). In other embodiments, R^{4d} is —Si(C₁-C₆ alkyl)₃. In other embodiments, R^{4d} is —C(O)O(C₁-C₆ alkyl). In other embodiments, R^{4d} is (C₁-C₆ alkyl)-(C₃-C₆ cycloalkyl)-. In other embodiments, R^{4d} is (C₁-C₆ haloalkyl)-(C₃-C₆ cycloalkyl)-. In other embodiments, R^{4d} is C₃-C₆ cycloalkyl, wherein said cycloalkyl is optionally substituted with one or more halogen or —CN. In some embodiments, the invention relates to a compound of formula (II-C), or a pharmaceutically acceptable salt thereof, wherein R^{4d} is Cl, —CH₃, —CH(CH₃)₂, —C(CH₃)₃, —CH₂C(CH₃)₃, —CF₃, —C(CH₃)₂(CF₃), —OCF₃, —CH(CH₂)(OCH₃), —Si(CH₃)₃, —C(O)OCH₂CH₃, cyclopropyl, 1-cyanocyclopropyl, 1-methylcyclopropyl, 1-trifluoromethylcyclopropyl, 1-methylcyclobutyl, or 3,3-difluorocyclobutyl. In other embodiments, R^{4d} is Cl. In other embodiments, R^{4d} is —CH₃. In other embodiments, R^{4d} is —CH(CH₃)₂. In other embodiments, R^{4d} is —C(CH₃)₃. In other embodiments, R^{4d} is —CH₂C(CH₃)₃. In other embodiments, R^{4d} is —CF₃. In other embodiments, R^{4d} is —C(CH₃)₂(CF₃). In other embodiments, R^{4d} is —OCF₃. In other embodiments, R^{4d} is —CH(CH₂)(OCH₃). In other embodiments, R^{4d} is —Si(CH₃)₃. In other embodiments, R^{4d} is —C(O)OCH₂CH₃. In other embodiments, R^{4d} is cyclopropyl. In other embodiments, R^{4d} is 1-cyanocyclopropyl. In other embodiments, R^{4d} is 1-methylcyclopropyl. In other embodiments, R^{4d} is 1-trifluoromethylcyclopropyl. In other embodiments, R^{4d} is 1-methylcyclobutyl. In other embodiments, R^{4d} is 3,3-difluorocyclobutyl.

In some embodiments, the invention relates to a compound of formula (III), or a pharmaceutically acceptable salt thereof, wherein R^{k} is H, —OH, or C₁-C₆ alkoxy.

In some embodiments, the invention relates to a compound of formula (III), or a pharmaceutically acceptable salt thereof, wherein X₃ₖ is N. In some embodiments, the invention relates to a compound of formula (III), or a pharmaceutically acceptable salt thereof, wherein X₃ₖ is CH.

In some embodiments, the invention relates to a compound of formula (III), or a pharmaceutically acceptable salt thereof, wherein X₄ₖ is N. In some embodiments, the invention relates to a compound of formula (III), or a pharmaceutically acceptable salt thereof, wherein X₄ₖ is CH.

In some embodiments, the invention relates to a compound of formula (III), or a pharmaceutically acceptable salt thereof, wherein X₅ₖ is N. In some embodiments, the invention relates to a compound of formula (III), or a pharmaceutically acceptable salt thereof, wherein X₅ₖ is CR^{5k}. In some embodiments, the invention relates to a compound of formula (III), or a pharmaceutically acceptable salt thereof, wherein X₅ₖ is CR^{5k} and R^{5k} is H, C₁-C₆ alkoxy, —OH, —OCH₂CH₂N(CH₃)₂, or —N(CH₃)(CH₂CH₂OCH₃). In other embodiments, R^{5k} is C₁-C₆ alkoxy. In some embodiments, the invention relates to a compound of formula (III), or a pharmaceutically acceptable salt thereof, wherein X₅ₖ is CR^{5k} and R^{5k} is H, —OH, —OCH₃, —OCH₂CH₂N(CH₃)₂, or —N(CH₃)(CH₂CH₂OCH₃). In other embodiments, R^{5k} is H. In other embodiments, R^{5k} is —OH. In other embodiments, R^{5k} is —OCH₃. In other embodiments, R^{5k} is —OCH₂CH₂N(CH₃)₂. In other embodiments, R^{5k} is —N(CH₃)(CH₂CH₂OCH₃).

In some embodiments, the invention relates to a compound of formula (III), or a pharmaceutically acceptable salt thereof, wherein X₆ₖ is N. In some embodiments, the invention relates to a compound of formula (III), or a pharmaceutically acceptable salt thereof, wherein X₆ₖ is N⁺—O⁻. In some embodiments, the invention relates to a compound of formula (III), or a pharmaceutically acceptable salt thereof, wherein X₆ₖ is CR^{6k} and R^{6k} is H, —OH, —OCH₃, or —C(O)NH₂. In some embodiments, the invention relates to a compound of formula (III), or a pharmaceutically acceptable salt thereof, wherein X₆ₖ is CR^{6k} and R^{6k} is H, —OH, or C₁-C₆ alkoxy. In other embodiments, R^{k} is C₁-C₆ alkoxy. In some embodiments, the invention relates to a compound of formula (III), or a pharmaceutically acceptable salt thereof, wherein X₆ₖ is CR^{6k} and R^{6k} is H, —OH, or —OCH₃. In other embodiments, R^{6k} is H. In other embodiments, R^{6k} is —OH. In other embodiments, R^{6k} is —OCH₃. In other embodiments, R^{6k} is —C(O)NH₂.

In some embodiments, the invention relates to a compound of formula (III), or a pharmaceutically acceptable salt thereof, wherein R^{2L} is —CH₃.

In some embodiments, the invention relates to a compound of formula (III), or a pharmaceutically acceptable salt thereof, wherein R^{4L} is C₁-C₆ alkyl, C₁-C₆ haloalkyl, or (C₁-C₆ haloalkyl)-(C₃-C₆ cycloalkyl)-. In other embodiments, R^{4L} is C₁-C₆ alkyl. In other embodiments, R^{4L} is C₁-C₆ haloalkyl. In other embodiments, R^{4L} is (C₁-C₆ haloalkyl)-(C₃-C₆ cycloalkyl)-. In some embodiments, the invention relates to a compound of formula (III), or a pharmaceutically acceptable salt thereof, wherein R^{4L} is —C(CH₃)₃, —C(CH₃)₂(CF₃), or 1-trifluoromethylcyclopropyl. In other embodiments, R^{4L} is —C(CH₃)₃. In other embodiments, R^{4L} is —C(CH₃)₂(CF₃). In other embodiments, R^{4L} is 1-trifluoromethylcyclopropyl.

In some embodiments, the invention relates to a compound of formula (III), or a pharmaceutically acceptable salt thereof, wherein R^{5L} is H, halo, or C₁-C₆ alkyl. In other embodiments, R^{5L} is halo. In other embodiments, R^{5L} is C₁-C₆ alkyl. In some embodiments, the invention relates to a compound of formula (III), or a pharmaceutically acceptable salt thereof, wherein R^{5L} is H, F, Cl, or —CH₃. In other embodiments, R^{5L} is H. In other embodiments, R^{5L} is F. In other embodiments, R^{5L} is Cl. In other embodiments, R^{5L} is —CH₃.

In some embodiments, the invention relates to a compound of formula (III-A):

or a pharmaceutically acceptable salt thereof, wherein:
X₃ₖ is N or CH;
X₄ₖ is N or CH;
X₅ₖ is N or CR^{5k};
X₆ₖ is N, N⁺—O⁻, or CR^{6k};
R^{5k} is H, C₁-C₆ alkoxy, —OH, or —OCH₂CH₂N(CH₃)₂;
R^{6k} is H, —OH, C₁-C₆ alkoxy, or —C(O)NH₂;
R^{2L} is C₁-C₆ alkyl;
R^{4L} is C₁-C₆ alkyl, C₁-C₆ haloalkyl, or (C₁-C₆ haloalkyl)-(C₃-C₆ cycloalkyl)-; and
R^{5L} is H, halo, or C₁-C₆ alkyl, provided that:
(i) no more than two of X₃ₖ, X₄ₖ, X₅ₖ, and X₆ₖ are N; and
(ii) if X₆ₖ is N⁺—O⁻, then X₃ₖ and X₄ₖ are CH, and X₅ₖ is CR^{5k}; and
(iii) if X₅ₖ is N, then X₄ₖ is N.

In some embodiments, the invention relates to a compound of formula (III-A), or a pharmaceutically acceptable salt thereof, wherein R^{6k} is H, —OH, or C₁-C₆ alkoxy.

In some embodiments, the invention relates to a compound of formula (III-A), or a pharmaceutically acceptable salt thereof, wherein X₃ₖ is N. In some embodiments, the invention relates to a compound of formula (III-A), or a pharmaceutically acceptable salt thereof, wherein X₃ₖ is CH.

In some embodiments, the invention relates to a compound of formula (III-A), or a pharmaceutically acceptable salt thereof, wherein X₄ₖ is N. In some embodiments, the invention relates to a compound of formula (III-A), or a pharmaceutically acceptable salt thereof, wherein X₄ₖ is CH.

In some embodiments, the invention relates to a compound of formula (III-A), or a pharmaceutically acceptable salt thereof, wherein X₅ₖ is N. In some embodiments, the invention relates to a compound of formula (III-A), or a pharmaceutically acceptable salt thereof, wherein X₅ₖ is CR^{5k}. In some embodiments, the invention relates to a compound of formula (III-A), or a pharmaceutically acceptable salt thereof, wherein X₅ₖ is CR^{5k} and R^{5k} is H, C₁-C₆ alkoxy, —OH, or —OCH₂CH₂N(CH₃)₂. In other embodiments, R^{5k} is C₁-C₆ alkoxy. In some embodiments, the invention relates to a compound of formula (III-A), or a pharmaceutically acceptable salt thereof, wherein X₅ₖ is CR^{5k} and R^{5k} is H, —OCH₃, —OH, or —OCH₂CH₂N(CH₃)₂. In other embodiments, R^{5k} is H. In other embodiments, R^{5k} is —OCH₃. In other embodiments, R^{5k} is —OH. In other embodiments, R^{5k} is —OCH₂CH₂N(CH₃)₂.

In some embodiments, the invention relates to a compound of formula (III-A), or a pharmaceutically acceptable salt thereof, wherein X₆ₖ is N. In some embodiments, the invention relates to a compound of formula (III-A), or a pharmaceutically acceptable salt thereof, wherein X₆ₖ is N⁺—O⁻. In some embodiments, the invention relates to a compound of formula (III-A), or a pharmaceutically acceptable salt thereof, wherein X₆ₖ is CR^{6k}. In some embodiments, the invention relates to a compound of formula (III-A), or a pharmaceutically acceptable salt thereof, wherein X₆ₖ is CR^{6k} and R^{k} is H, —OH, —OCH₃, or —C(O)NH₂. In some embodiments, the invention relates to a compound of formula (III-A), or a pharmaceutically acceptable salt thereof, wherein X₆ₖ is CR^{6k} and R^{k} is H, —OH, or C₁-C₆ alkoxy. In other embodiments, C₁-C₆ alkoxy. In some embodiments, the invention relates to a compound of formula (III-A), or a pharmaceutically acceptable salt thereof, wherein X₆ₖ is CR^{6k} and R^{6k} is H, —OH, or —OCH₃. In other embodiments, R^{6k} is H. In other embodiments, R^{6k} is —OH. In other embodiments, R^{6k} is —OCH₃. In other embodiments, R^{6k} is —C(O)NH₂.

In some embodiments, the invention relates to a compound of formula (III-A), or a pharmaceutically acceptable salt thereof, wherein R^{2L} is —CH₃.

In some embodiments, the invention relates to a compound of formula (III-A), or a pharmaceutically acceptable salt thereof, wherein R^{4L} is C₁-C₆ alkyl, C₁-C₆ haloalkyl, or (C₁-C₆ haloalkyl)-(C₃-C₆ cycloalkyl)-. In other embodiments, R^{4L} is C₁-C₆ alkyl. In other embodiments, R^{4L} is C₁-C₆ haloalkyl. In other embodiments, R^{4L} is (C₁-C₆ haloalkyl)-(C₃-C₆ cycloalkyl)-. In some embodiments, the invention relates to a compound of formula (III-A), or a pharmaceutically acceptable salt thereof, wherein R^{4L} is —C(CH₃)₃, —C(CH₃)₂(CF₃), or 1-trifluoromethylcyclopropyl. In other embodiments, R^{4L} is —C(CH₃)₃. In other embodiments, R^{4L} is —C(CH₃)₂(CF₃). In other embodiments, R^{4L} is 1-trifluoromethylcyclopropyl.

In some embodiments, the invention relates to a compound of formula (III-A), or a pharmaceutically acceptable salt thereof, wherein R^{5L} is H, halo, or C₁-C₆ alkyl. In other embodiments, R^{5L} is halo. In other embodiments, R^{5L} is C₁-C₆ alkyl. In some embodiments, the invention relates to a compound of formula (III-A), or a pharmaceutically acceptable salt thereof, wherein R^{5L} is H, F, Cl, or —CH₃. In other embodiments, R^{5L} is H. In other embodiments, R^{5L} is F. In other embodiments, R^{5L} is Cl. In other embodiments, R^{5L} is —CH₃.

In some embodiments, the invention relates to a compound selected from Table A, or a pharmaceutically acceptable salt thereof. In other embodiments, the invention relates to a compound selected from Table A, i.e., the compound in non-salt form.

TABLE A
Compound Structures and Names.

| | |
|---|---|
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |
| | |

In some embodiments, the invention relates to a compound selected from Table B, or a pharmaceutically acceptable salt thereof. In other embodiments, the invention relates to a compound selected from Table B, i.e., the compound in non-salt form.

```
TABLE B
Compound Structures and Names.
  2-(5-chloro-4-cyclobutyl-2-methyl-phenyl)-4-oxo- 1H-1,6-naphthyridine-5-carboxamide
  2-[5-chloro-2-methyl-4-(1- methylcyclopropyl)phenyl]-4-oxo-1H-1,6- naphthyridine-5-carboxamide
  2-[5-chloro-4-(4,4-difluoro-1-methyl-cyclohexyl)-2- methyl-phenyl]-4-oxo-1H-1,6-naphthyridine-5- carboxamide
  2-[5-chloro-2-methyl-4-(1- methylcyclopentyl)phenyl]-4-oxo-1H-1,6- naphthyridine-5-carboxamide
  2-[5-chloro-2-methyl-4-(1- methylcyclobutyl)phenyl]-4-oxo-1H-1,6- naphthyridine-5-carboxamide
  2-[5-chloro-2-methyl-4-[1- (trifluoromethyl)cyclopropyl]phenyl]-4-oxo-1H- 1,6-naphthyridine-5-carboxamide
  2-[5-fluoro-2-methyl-4-(1- methylcyclopentyl)phenyl]-4-oxo-1H-1,6- naphthyridine-5-carboxamide
  2-[5-chloro-4-(3,3-difluoro-1-methyl-cyclobutyl)-2- methyl-phenyl]-4-oxo-1H-1,6-naphthyridine-5- carboxamide
  2-(5-chloro-2-methyl-4-phenyl-phenyl)-4-oxo-1H- 1,6-naphthyridine-5-carboxamide
  2-(5-chloro-4-cyclopentyl-2-methyl-phenyl)-4-oxo- 1H-1,6-naphthyridine-5-carboxamide
  2-[5-chloro-2-methyl-4-[2-(trifluoromethyl)oxetan- 2-yl]phenyl]-4-oxo-1H-1,6-naphthyridine-5- carboxamide
  (R)-2-(5-chloro-2-methyl-4-(2- (trifluoromethyl)oxetan-2-yl)phenyl)-4-oxo-1,4- dihydro-1,6-naphthyridine-5-carboxamide
  (S)-2-(5-chloro-2-methyl-4-(2- (trifluoromethyl)oxetan-2-yl)phenyl)-4-oxo-1,4- dihydro-1,6-naphthyridine-5-carboxamide
  2-[4-(1-bicyclo[1.1.1]pentanyl)-5-chloro-2-methyl- phenyl]-4-oxo-1H-1,6-naphthyridine-5-carboxamide
  2-[5-chloro-4-(2,2-difluorospiro[3.3]heptan-6-yl)-2- methyl-phenyl]-4-oxo-1H-1,6-naphthyridine-5- carboxamide
  2-(5-chloro-2-methyl-4-spiro[2.3]hexan-5-yl- phenyl)-4-oxo-1H-1,6-naphthyridine-5-carboxamide
  2-[4-(1-bicyclo[3.1.0]hexanyl)-5-chloro-2-methyl- phenyl]-4-oxo-1H-1,6-naphthyridine-5-carboxamide
  2-(4-((1R,5R)-bicyclo[3.1.0]hexan-1-yl)-5-chloro-2- methylphenyl)-4-oxo-1,4-dihydro-1,6- naphthyridine-5-carboxamide
  2-(4-((1S,5S)-bicyclo[3.1.0]hexan-1-yl)-5-chloro-2- methylphenyl)-4-oxo-1,4-dihydro-1,6- naphthyridine-5-carboxamide
  2-(5-chloro-2-methyl-4-norbornan-2-yl-phenyl)-4- oxo-1H-1,6-naphthyridine-5-carboxamide
  2-[3-chloro-2-fluoro-6-methyl-4-(2,2,2-trifluoro-1,1- dimethyl-ethyl)phenyl]-4-oxo-1H-1,6- naphthyridine-5-carboxamide
  2-[4-(1-bicyclo[2.2.2]octanyl)-5-chloro-2-methyl- phenyl]-4-oxo-1H-1,6-naphthyridine-5-carboxamide
  2-[5-chloro-4-(2,2-difluoro-1,1-dimethyl-ethyl)-2- methyl-phenyl]-4-oxo-1H-1,6-naphthyridine-5- carboxamide
  2-[5-chloro-2-methyl-4-(trifluoromethoxy)phenyl]- 4-oxo-1H-1,6-naphthyridine-5-carboxamide
  2-[5-chloro-2-methyl-4-[1- (trifluoromethyl)cyclopentyl]phenyl]-4-oxo-1H- 1,6-naphthyridine-5-carboxamide
  2-[4-(3-bicyclo[3.1.0]hexanyl)-5-chloro-2-methyl- phenyl]-4-oxo-1H-1,6-naphthyridine-5-carboxamide
  2-(4-((1R,3r,5S)-bicyclo[3.1.0]hexan-3-yl)-5- chloro-2-methylphenyl)-4-oxo-1,4-dihydro-1,6- naphthyridine-5-carboxamide
  2-(4-((1R,3s,5S)-bicyclo[3.1.0]hexan-3-yl)-5- chloro-2-methylphenyl)-4-oxo-1,4-dihydro-1,6- naphthyridine-5-carboxamide
  2-(5-chloro-2-methyl-4-trimethylsilyl-phenyl)-4- oxo-1H-1,6-naphthyridine-5-carboxamide
  2-(5-chloro-2-methyl-6-phenyl-3-pyridyl)-4-oxo- 1H-1,6-naphthyridine-5-carboxamide
  2-[6-(1-bicyclo[2.2.2]octanyl)-5-chloro-2-methyl-3- pyridyl]-4-oxo-1H-1,6-naphthyridine-5- carboxamide
  2-(5-chloro-6-dispiro[2.0.2⁴.1³]heptan-7-yl-2- methyl-3-pyridyl)-4-oxo-1H-1,6-naphthyridine-5- carboxamide
  2-[6-(1-bicyclo[1.1.1]pentanyl)-5-chloro-2-methyl- 3-pyridyl]-4-oxo-1H-1,6-naphthyridine-5- carboxamide
  2-[6-(1-bicyclo[2.1.1]hexanyl)-5-chloro-2-methyl-3- pyridyl]-4-oxo-1H-1,6-naphthyridine-5- carboxamide
  2-[5-chloro-6-(3,3-difluoro-1-methyl-cyclobutyl)-2- methyl-3-pyridyl]-4-oxo-1H-1,6-naphthyridine-5- carboxamide
  2-[5-chloro-6-(3,3-difluoro-1-methyl-cyclopentyl)- 2-methyl-3-pyridyl]-4-oxo-1H-1,6-naphthyridine-5- carboxamide
  (R)-2-(5-chloro-6-(3,3-difluoro-1- methylcyclopentyl)-2-methylpyridin-3-yl)-4-oxo- 1,4-dihydro-1,6-naphthyridine-5-carboxamide
  (S)-2-(5-chloro-6-(3,3-difluoro-1- methylcyclopentyl)-2-methylpyridin-3-yl)-4-oxo- 1,4-dihydro-1,6-naphthyridine-5-carboxamide
  2-(5-chloro-2-methyl-6-spiro[3.3 ]heptan-2-yl-3- pyridyl)-4-oxo-1H-1,6-naphthyridine-5- carboxamide
  2-[5-chloro-2-methyl-6-(2,2,2-trifluoro-1,1- dimethyl-ethyl)-3-pyridyl]-4-oxo-1H-1,6- naphthyridine-5-carboxamide
  2-[5-chloro-2-methyl-6-(1-methylcyclobutyl)-3- pyridyl]-4-oxo-1H-1,6-naphthyridine-5- carboxamide
  2-[5-chloro-2-methyl-6-(3-methyl-1- bicyclo[1.1.1]pentanyl)-3-pyridyl]-4-oxo-1H-1,6- naphthyridine-5-carboxamide
  2-(5-chloro-2-methyl-6-norbornan-1-yl-3-pyridyl)- 4-oxo-1H-1,6-naphthyridine-5-carboxamide
  2-[5-chloro-2-methyl-6-(1-methylcyclopentyl)-3- pyridyl]-4-oxo-1H-1,6-naphthyridine-5- carboxamide
  2-[5-chloro-2-methyl-6-(1-methylcyclopropyl)-3- pyridyl]-4-oxo-1H-1,6-naphthyridine-5- carboxamide
  2-[5-chloro-6-(2,2-dimethylpyrrolidin-1-yl)-2- methyl-3-pyridyl]-4-oxo-1H-1,6-naphthyridine-5- carboxamide
  2-(4-tert-butyl-3,5-difluoro-2-methyl-phenyl)-4- oxo-1H-1,6-naphthyridine-5-carboxamide
  2-(4-tert-butyl-5-chloro-3-fluoro-2-methyl- phenyl)-4-oxo-1H-1,6-naphthyridine-5- carboxamide
  2-(4-tert-butyl-3-chloro-2-fluoro-6-methyl- phenyl)-4-oxo-1H-1,6-naphthyridine-5- carboxamide
  2-(4-tert-butyl-5-fluoro-2-methyl-phenyl)-4-oxo- 1H-1,6-naphthyridine-5-carboxamide
  2-(4-tert-butyl-2,3-difluoro-6-methyl-phenyl)-4- oxo-1H-1,6-naphthyridine-5-carboxamide
  methyl 5-tert-butyl-2-(5-carbamoyl-4-oxo-1H- 1,6-naphthyridin-2-yl)-4-chloro-benzoate
  2-[4-tert-butyl-5-chloro-2-(hydroxymethyl) phenyl]-4-oxo-1H-1,6-naphthyridine-5- carboxamide
  2-[4-(2-hydroxy-1,1-dimethyl-ethyl)-2-methyl-5- (trifluoromethyl)phenyl]-4-oxo-1H-1,6- naphthyridine-5-carboxamide
  2-(4-tert-butyl-5-chloro-2-methyl-phenyl)-6-oxido- 1H-1,6-naphthyridin-6-ium-4-one
  2-[5-chloro-6-(2-hydroxy-1,1-dimethyl-ethyl)-2- methyl-3-pyridyl]-4-oxo-1H-1,6-naphthyridine-5- carboxamide
  2-[6-(1-bicyclo[1.1.1]pentanyl)-5-chloro-2-methyl- 3-pyridyl]-4-oxo-1H-1,6-naphthyridine-5- carbonitrile
  2-(5-chloro-2-methyl-6-spiro[3.3]heptan-2-yl-3- pyridyl)-4-oxo-1H-1,6-naphthyridine-5-carbonitrile
  2-(5-chloro-2-methyl-3-pyridyl)-4-oxo-1H-1,6- naphthyridine-5-carboxamide
  2-(5-chloro-2-methyl-6-norbornan-1-yl-3-pyridyl)- 4-oxo-1H-1,6-naphthyridine-5-carbonitrile
  2-(5-chloro-2-methyl-6-norbornan-2-yl-3-pyridyl)- 4-oxo-1H-1,6-naphthyridine-5-carboxamide
  2-[5-(5-carbamoyl-4-oxo-1H-1,6-naphthyridin-2- yl)-3-chloro-6-methyl-2-pyridyl]-2-methyl- propanoic acid
  2-[4-(2-adamantyl)-5-chloro-2-methyl-phenyl]-4- oxo-1H-1,6-naphthyridine-5-carboxamide
  2-[5-chloro-4-(2-fluoro-2-methyl-propyl)-2-methyl- phenyl]-4-oxo-1H-1,6-naphthyridine-5-carboxamide
  2-[5-chloro-2-methyl-4-(3-methyl-3- bicyclo[3.1.0]hexanyl)phenyl]-4-oxo-1H-1,6- naphthyridine-5-carboxamide
  2-(5-chloro-2-methyl-4-((1R,3r,5S)-3- methylbicyclo[3.1.0]hexan-3-yl)phenyl)-4-oxo-1,4- dihydro-1,6-naphthyridine-5-carboxamide
  2-(5-chloro-2-methyl-4-((1R,3s,5S)-3- methylbicyclo[3.1.0]hexan-3-yl)phenyl)-4-oxo-1,4- dihydro-1,6-naphthyridine-5-carboxamide
  2-[4-(5-carbamoyl-4-oxo-1H-1,6-naphthyridin-2- yl)-5-methyl-2-(trifluoromethyl)phenyl]-2-methyl- propanoic acid
  2-[5-chloro-2-methyl-4-[2,2,2-trifluoro-1- (hydroxymethyl)-1-methyl-ethyl]phenyl]-4-oxo-1H- 1,6-naphthyridine-5-carboxamide
  (R)-2-(5-chloro-2-methyl-4-(1,1,1-trifluoro-3- hydroxy-2-methylpropan-2-yl)phenyl)-4-oxo-1,4- dihydro-1,6-naphthyridine-5-carboxamide
  (S)-2-(5-chloro-2-methyl-4-(1,1,1-trifluoro-3- hydroxy-2-methylpropan-2-yl)phenyl)-4-oxo-1,4- dihydro-1,6-naphthyridine-5-carboxamide
  2-[4-(5-carbamoyl-4-oxo-1H-1,6-naphthyridin-2- yl)-2-chloro-5-methyl-phenyl]-3,3,3-trifluoro-2- methyl-propanoic acid
  (S)-2-(4-(5-carbamoyl-4-oxo-1,4-dihydro-1,6- naphthyridin-2-yl)-2-chloro-5-methylphenyl)-3,3,3- trifluoro-2-methylpropanoic acid
  (R)-2-(4-(5-carbamoyl-4-oxo-1,4-dihydro-1,6- naphthyridin-2-yl)-2-chloro-5-methylphenyl)-3,3,3- trifluoro-2-methylpropanoic acid
  2-(4-tert-butyl-5-chloro-2-methyl-phenyl)-5- methoxy-1H-1,6-naphthyridin-4-one
  [2-(4-tert-butyl-5-chloro-2-methyl-phenyl)-4-oxo- 1H-1,6-naphthyridin-5-yl]urea
  2-(4-tert-butyl-5-chloro-2-methyl-phenyl)-5- hydroxy-1H-1,6-naphthyridin-4-one
  1-[2-(4-tert-butyl-5-chloro-2-methyl-phenyl)-4-oxo- 1H-1,6-naphthyridin-5-yl]-3-methyl-urea
  2-(4-tert-butyl-5-chloro-2-methyl-phenyl)-N- methyl-4-oxo-1H-1,6-naphthyridine-5-carboxamide
  2-(4-tert-butyl-5-chloro-2-methyl-phenyl)-5- (dimethylamino)-1H-1,6-naphthyridin-4-one
  2-(4-tert-butyl-5-chloro-2-methyl-phenyl)-4-oxo- 1H-1,6-naphthyridine-5-carboxamidine
  2-(4-tert-butyl-5-chloro-2-methyl-phenyl)-8-methyl- 4-oxo-1H-1,6-naphthyridine-3-carboxylic acid
  2-(4-tert-butyl-5-chloro-2-methyl-phenyl)-8- methyl-1H-1,6-naphthyridin-4-one
  2-(4-tert-butyl-5-chloro-2-methyl-phenyl)-8- methyl-4-oxo-1H-1,6-naphthyridine-5- carboxamide
  2-(4-tert-butyl-5-chloro-2-methyl-phenyl)-5-(1- methylimidazol-2-yl)-1H-1,6-naphthyridin-4-one
  2-(6-tert-butyl-5-chloro-2-methyl-3-pyridyl)-5-(1- methylimidazol-2-yl)-1H-1,6-naphthyridin-4-one
  2-(4-tert-butyl-5-chloro-2-methyl-phenyl)-5- oxazol-2-yl-1H-quinolin-4-one
  2-(4-tert-butyl-5-chloro-2-methyl-phenyl)-5-(5- chloro-1-methyl-imidazol-2-yl)-1H-1,6- naphthyridin-4-one
  2-(4-tert-butyl-5-chloro-2-methyl-phenyl)-5- (2-pyridyl)-1H-1,6-naphthyridin-4-one
  2-(4-tert-butyl-5-chloro-2-methyl-phenyl)-5-(3- methyl-2-pyridyl)-1H-1,6-naphthyridin-4-one
  2-(4-tert-butyl-5-chloro-2-methyl-phenyl)-5- pyrazin-2-yl-1H-1,6-naphthyridin-4-one
  2-(4-tert-butyl-5-chloro-2-methyl-phenyl)-5- (6-methyl-2-pyridyl)-1H-1,6-naphthyridin-4- one
  2-(4-tert-butyl-5-chloro-2-methyl-phenyl)-5-(4- methyloxazol-5-yl)-1H-1,6-naphthyridin-4-one
  2-(4-tert-butyl-5-chloro-2-methyl-phenyl)-5-(4- methyl-1H-pyrazol-3-yl)-1H-1,6-naphthyridin-4- one
  2-(4-tert-butyl-5-chloro-2-methyl-phenyl)-5-(1,5- dimethyltriazol-4-yl)-1H-1,6-naphthyridin-4-one
  2-(4-tert-butyl-5-chloro-2-methyl-phenyl)-5-(2,4- dimethyloxazol-5-yl)-1H-1,6-naphthyridin-4-one
  ethyl 5-[2-(4-tert-butyl-5-chloro-2-methyl-phenyl)- 4-oxo-1H-1,6-naphthyridin-5-yl]-4-methyl- oxazole-2-carboxylate
  2-(4-tert-butyl-5-chloro-2-methyl-phenyl)-5-(2,5- dimethylthiazol-4-yl)-1H-1,6-naphthyridin-4-one
  2-(4-tert-butyl-5-chloro-2-methyl-phenyl)-5- [1-(trifluoromethyl)pyrazol-3-yl]-1H-1,6- naphthyridin-4-one
  2-(4-tert-butyl-5-chloro-2-methyl-phenyl)-5-(3,5- dimethylpyrazol-1-yl)-1H-1,6-naphthyridin-4-one
  2-(4-tert-butyl-5-chloro-2-methyl-phenyl)-5-[5- (trifluoromethyl)pyrazol-1-yl]-1H-1,6-naphthyridin- 4-one
  2-(4-tert-butyl-5-chloro-2-methyl-phenyl)-5-(5- methylpyrazol-1-yl)-1H-1,6-naphthyridin-4-one
  2-(4-tert-butyl-5-chloro-2-methyl-phenyl)-5-(1,4- dimethylimidazol-2-yl)-1H-1,6-naphthyridin-4-one
  2-(4-tert-butyl-5-chloro-2-methyl-phenyl)-5-(4- methyl-1,2,4-triazol-3-yl)-1H-1,6-naphthyridin- 4-one
  2-(4-tert-butyl-5-chloro-2-methyl-phenyl)-5-(5- chloro-2-methyl-1,2,4-triazol-3-yl)-1H-1,6- naphthyridin-4-one
  2-(4-tert-butyl-2,5-dimethyl-phenyl)-5-(5-chloro- 2-methyl-1,2,4-triazol-3-yl)-1H-1,6-naphthyridin- 4-one
  2-(4-tert-butyl-5-chloro-2-methyl-phenyl)-5-(4,5- dimethyl-1,2,4-triazol-3-yl)-1H-1,6-naphthyridin- 4-one
  2-(6-tert-butyl-5-chloro-2-methyl-3-pyridyl)-5- (1,4-dimethylimidazol-2-yl)-1H-1,6-naphthyridin- 4-one
  2-(6-tert-butyl-5-chloro-2-methyl-3-pyridyl)-5-(2- methyl-1,2,4-triazol-3-yl)-1H-1,6-naphthyridin-4- one
  2-[5-chloro-2-methyl-4-(2,2,2-trifluoro-1,1- dimethyl-ethyl)phenyl]-5-(1,4-dimethylimidazol-2- yl)-1H-1,6-naphthyridin-4-one
  2-[5-chloro-2-methyl-4-(2,2,2-trifluoro-1,1- dimethyl-ethyl)phenyl]-5-(2,5-dimethyl-1,2,4- triazol-3-yl)-1H-1,6-naphthyridin-4-one
  2-(6-tert-butyl-5-chloro-2-methyl-3-pyridyl)-5- (2,5-dimethyl-1,2,4-triazol-3-yl)-1H-1,6- naphthyridin-4-one
  2-(4-tert-butyl-5-chloro-2-methyl-phenyl)-5-(1- methylimidazo[4,5-b]pyridin-2-yl)-1H-1,6- naphthyridin-4-one
  2-(4-tert-butyl-2,5-dimethyl-phenyl)-5-(1,5- dimethylimidazol-2-yl)-1H-1,6-naphthyridin-4- one
  2-(4-tert-butyl-5-chloro-2-methyl-phenyl)-5-(1,4- dimethylpyrazol-3-yl)-1H-1,6-naphthyridin-4-one
  2-(4-tert-butyl-5-chloro-2-methyl-phenyl)-5-(1,5- dimethylimidazol-2-yl)-1H-1,6-naphthyridin-4-one
  2-(4-tert-butyl-5-chloro-2-methyl-phenyl)-5-(2,5- dimethyl-1,2,4-triazol-3-yl)-1H-1,6-naphthyridin-4- one
  6-[2-(4-tert-butyl-5-chloro-2-methyl-phenyl)-4-oxo- 1H-1,6-naphthyridin-5-yl]pyridine-2-carboxamide
  2-(4-tert-butyl-5-chloro-2-methyl-phenyl)-5-(2- methylpyrimidin-4-yl)-1H-1,6-naphthyridin-4- one
  2-(4-tert-butyl-5-chloro-2-methyl-phenyl)-5- (2-oxo-1H-pyrimidin-4-yl)-1H-1,6- naphthyridin-4-one
  2-(4-tert-butyl-5-chloro-2-methyl-phenyl)-4-oxo- 1H-pyrido[2,3-d]pyridazine-5-carboxamide
  2-(5-tert-butyl-4-chloro-2-methyl-pyrazol-3-yl)- 4-oxo-1H-1,6-naphthyridine-5-carboxamide
  2-(5-tert-butyl-4-chloro-2-isopropyl-pyrazol-3- yl)-4-oxo-1H-1,6-naphthyridine-5-carboxamide
  2-(5-tert-butyl-2-methyl-pyrazol-3-yl)-6-fluoro-1H- quinolin-4-one
  2-(5-tert-butyl-2-methyl-pyrazol-3-yl)-4-oxo-1H- 1,6-naphthyridine-5-carboxamide
  methyl 2-(4-(tert-butyl)-2-methylphenyl)-4- oxo-1,4-dihydro-1,6-naphthyridine-3- carboxylate
  2-(4-(tert-butyl)-5-chloro-2-methylphenyl)-5-(1- methyl-1H-1,2,4-triazol-5-yl)-1,6-naphthyridin- 4(1H)-one
  2-(5-chloro-4-(3,3-difluoro-1-methylcyclopentyl)-2- methylphenyl)-4-oxo-1,4-dihydro-1,6- naphthyridine-5-carboxamide
  (S)-2-(5-chloro-4-(3,3-difluoro-1- methylcyclopentyl)-2-methylphenyl)-4-oxo-1,4- dihydro-1,6-naphthyridine-5-carboxamide
  (R)-2-(5-chloro-4-(3,3-difluoro-1- methylcyclopentyl)-2-methylphenyl)-4-oxo-1,4- dihydro-1,6-naphthyridine-5-carboxamide
  2-(4-(tert-butyl)-5-chloro-2-methylphenyl)-4- oxo-1,4-dihydro-1,6-naphthyridine-5- sulfonamide
  2-(2-methyl-4-(2-(methyl-d₃)propan-2-yl- 1,1,1,3,3,3-d₆)-5-(trifluoromethyl)phenyl)-4-oxo- 1,4-dihydro-1,6-naphthyridine-5-carboxamide
  2-(5-chloro-2-methyl-6-(2-(methyl-d₃)propan-2-yl- 1,1,1,3,3,3-d₆)pyridin-3-yl)-4-oxo-1,4-dihydro-1,6- naphthyridine-5-carboxamide
  2-(3-chloro-2-fluoro-6-methyl-4-(2-(methyl- d₃)propan-2-yl-1,1,1,3,3,3-d₆)phenyl)-4-oxo-1,4- dihydro-1,6-naphthyridine-5-carboxamide
  2-(5-chloro-2-methyl-4-(1-(methyl- d₃)cyclopropyl)phenyl)-4-oxo-1,4-dihydro-1,6- naphthyridine-5-carboxamide
  2-(5-chloro-2-methyl-4-(2-(methyl-d₃)propan-2-yl- 1,1,1,3,3,3-d₆)phenyl-3-d)-4-oxo-1,4-dihydro-1,6- naphthyridine-5-carboxamide
  2-(5-chloro-2-methyl-4-(1,1,1-trifluoro-2- methylpropan-2-yl-3-¹³C-3,3,3-d₃)phenyl-3,6-d₂)-4- oxo-1,4-dihydro-1,6-naphthyridine-5-carboxamide
  2-(5-chloro-2-methyl-4-(1,1,1-trifluoro-2- methylpropan-2-yl-3,3,3-d3)phenyl-3,6-d2)-4-oxo- 1,4-dihydro-1,6-naphthyridine-5-carboxamide
  2-(2-methyl-4-(2-(methyl-d₃)propan-2-yl- 1,1,1,3,3,3-d₆)-5-(trifluoromethyl)phenyl)-4-oxo- 1,4-dihydro-1,6-naphthyridine-5-carboxamide
  2-(5-chloro-2-methyl-6-(2-(methyl-d₃)propan-2-yl- 1,1,1,3,3,3-d₆)pyridin-3-yl)-4-oxo-1,4-dihydro-1,6- naphthyridine-5-carboxamide
  2-(3-chloro-2-fluoro-6-methyl-4-(2-(methyl- d₃)propan-2-yl-1,1,1,3,3,3-d₆)phenyl)-4-oxo-1,4- dihydro-1,6-naphthyridine-5-carboxamide
  2-(5-chloro-2-methyl-4-(1-(methyl- d₃)cyclopropyl)phenyl)-4-oxo-1,4-dihydro-1,6- naphthyridine-5-carboxamide
  2-(5-chloro-2-methyl-4-(2-(methyl-d₃)propan-2-yl- 1,1,1,3,3,3-d₆)phenyl-3-d)-4-oxo-1,4-dihydro-1,6- naphthyridine-5-carboxamide
  2-(5-chloro-2-methyl-4-(1,1,1-trifluoro-2- methylpropan-2-yl-3-¹³C-3,3,3-d₃)phenyl-3,6-d₂)-4- oxo-1,4-dihydro-1,6-naphthyridine-5-carboxamide
  2-(5-chloro-2-methyl-4-(1,1,1-trifluoro-2- methylpropan-2-yl-3,3,3-d3)phenyl-3,6-d2)-4-oxo- 1,4-dihydro-1,6-naphthyridine-5-carboxamide
  2-(5-chloro-4-(1-hydroxy-2-(methyl-d3)propan- 2-yl-1,1,3,3,3-d5)-2-methylphenyl)-4-oxo-1,4- dihydro-1,6-naphthyridine-5-carboxamide
```

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(4-tert-butyl-5-chloro-2-methyl-phenyl)-4-oxo-1H-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-[5-chloro-2-methyl-4-(2,2,2-trifluoro-1,1-dimethyl-ethyl)phenyl]-4-oxo-1H-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(6-tert-butyl-5-chloro-2-methyl-3-pyridyl)-4-oxo-1H-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-[4-tert-butyl-2-methyl-5-(trifluoromethyl)phenyl]-4-oxo-1H-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(4-tert-butyl-2-fluoro-3,6-dimethyl-phenyl)-4-oxo-1H-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(4-tert-butyl-2,5-dimethyl-phenyl)-4-oxo-1H-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-[2,5-dimethyl-4-(2,2,2-trifluoro-1,1-dimethyl-ethyl)phenyl]-4-oxo-1H-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 4-oxo-2-(1,1,7-trimethyltetralin-6-yl)-1H-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-[4-isopropyl-2-methyl-5-(trifluoromethyl)phenyl]-4-oxo-1H-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound selected from:

or a pharmaceutically acceptable salt thereof.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(4-(tert-butyl)-3-chloro-2-fluoro-6-methylphenyl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(5-chloro-4-cyclobutyl-2-methylphenyl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(5-chloro-2-methyl-4-(1-methylcyclopropyl)phenyl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(4-(bicyclo[1.1.1]pentan-1-yl)-5-chloro-2-methylphenyl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(4-(tert-butyl)-3,5-difluoro-2-methylphenyl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(5-chloro-6-(3,3-difluoro-1-methylcyclopentyl)-2-methylpyridin-3-yl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (R)-2-(5-chloro-6-(3,3-difluoro-1-methylcyclopentyl)-2-methylpyridin-3-yl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (S)-2-(5-chloro-6-(3,3-difluoro-1-methylcyclopentyl)-2-methylpyridin-3-yl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound rel-(R)-2-(5-chloro-6-(3,3-difluoro-1-methylcyclopentyl)-2-methylpyridin-3-yl)-4-oxo-1,4-dihydro-1,6-naphthyridine-5-carboxamide. In some embodiments, the invention relates to the compound rel-(R)-2-(5-chloro-6-(3,3-difluoro-1-methylcyclopentyl)-2-methylpyridin-3-yl)-4-oxo-1,4-dihydro-1,6-naphthyridine-5-carboxamide, wherein the compound has the stereochemistry corresponding to the first eluting isomer when the two diastereomers rel-(R)-4-benzyloxy-2-[5-chloro-6-(3,3-difluoro-1-methyl-cyclopentyl)-2-methyl-3-pyridyl]-1,6-naphthyridine-5-carbonitrile and rel-(S)-4-benzyloxy-2-[5-chloro-6-(3,3-difluoro-1-methyl-cyclopentyl)-2-methyl-3-pyridyl]-1,6-naphthyridine-5-carbonitrile are separated by SFC as described in Example 85. In other embodiments, the invention relates to the foregoing compound in non-salt form. Such compound is considered to be a “compound of the invention,” as that term is used herein.

In some embodiments, the invention relates to the compound rel-(S)-2-(5-chloro-6-(3,3-difluoro-1-methylcyclopentyl)-2-methylpyridin-3-yl)-4-oxo-1,4-dihydro-1,6-naphthyridine-5-carboxamide. In some embodiments, the invention relates to the compound rel-(S)-2-(5-chloro-6-(3,3-difluoro-1-methylcyclopentyl)-2-methylpyridin-3-yl)-4-oxo-1,4-dihydro-1,6-naphthyridine-5-carboxamide, wherein the compound has the stereochemistry corresponding to the second eluting isomer when the two diastereomers rel-(R)-4-benzyloxy-2-[5-chloro-6-(3,3-difluoro-1-methyl-cyclopentyl)-2-methyl-3-pyridyl]-1,6-naphthyridine-5-carbonitrile and rel-(S)-4-benzyloxy-2-[5-chloro-6-(3,3-difluoro-1-methyl-cyclopentyl)-2-methyl-3-pyridyl]-1,6-naphthyridine-5-carbonitrile are separated by SFC as described in Example 85. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(3-chloro-2-fluoro-6-methyl-4-(1,1,1-trifluoro-2-methylpropan-2-yl)phenyl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(5-chloro-2-methyl-6-(3-methylbicyclo[1.1.1]pentan-1-yl)pyridin-3-yl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(4-(tert-butyl)-5-chloro-3-fluoro-2-methylphenyl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(4-(bicyclo[3.1.0]hexan-3-yl)-5-chloro-2-methylphenyl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound 2-(5-chloro-4-(3,3-difluoro-1-methylcyclopentyl)-2-methylphenyl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (S)-2-(5-chloro-4-(3,3-difluoro-1-methylcyclopentyl)-2-methylphenyl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to a compound of formula

or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound (R)-2-(5-chloro-4-(3,3-difluoro-1-methylcyclopentyl)-2-methylphenyl)-4-oxo-1,4-dihydro-1,6-naphthyridine-5-carboxamide, or a pharmaceutically acceptable salt thereof. In other embodiments, the invention relates to the foregoing compound in non-salt form. Such compound is considered to be a “compound of the invention,” as that term is used herein.

In some embodiments, the invention relates to the compound rel-(S)-2-(5-chloro-4-(3,3-difluoro-1-methylcyclopentyl)-2-methylphenyl)-4-oxo-1,4-dihydro-1,6-naphthyridine-5-carboxamide. In some embodiments, the invention relates to the compound rel-(S)-2-(5-chloro-4-(3,3-difluoro-1-methylcyclopentyl)-2-methylphenyl)-4-oxo-1,4-dihydro-1,6-naphthyridine-5-carboxamide, wherein the compound has the stereochemistry corresponding to the first eluting isomer when the two diastereomers rel-(S)-2-[5-chloro-4-(3,3-difluoro-1-methyl-cyclopentyl)-2-methyl-phenyl]-4-oxo-1H-1,6-naphthyridine-5-carboxamide and rel-(R)-2-[5-chloro-4-(3,3-difluoro-1-methyl-cyclopentyl)-2-methyl-phenyl]-4-oxo-1H-1,6-naphthyridine-5-carboxamide are separated by SFC as described in Example 84. In other embodiments, the invention relates to the foregoing compound in non-salt form. Such compound is considered to be a “compound of the invention,” as that term is used herein.

In some embodiments, the invention relates to the compound rel-(R)-2-(5-chloro-4-(3,3-difluoro-1-methylcyclopentyl)-2-methylphenyl)-4-oxo-1,4-dihydro-1,6-naphthyridine-5-carboxamide. In some embodiments, the invention relates to the compound rel-(R)-2-(5-chloro-4-(3,3-difluoro-1-methylcyclopentyl)-2-methylphenyl)-4-oxo-1,4-dihydro-1,6-naphthyridine-5-carboxamide, wherein the compound has the stereochemistry corresponding to the second eluting isomer when the two diastereomers rel-(S)-2-[5-chloro-4-(3,3-difluoro-1-methyl-cyclopentyl)-2-methyl-phenyl]-4-oxo-1H-1,6-naphthyridine-5-carboxamide and rel-(R)-2-[5-chloro-4-(3,3-difluoro-1-methyl-cyclopentyl)-2-methyl-phenyl]-4-oxo-1H-1,6-naphthyridine-5-carboxamide are separated by SFC as described in Example 84. In other embodiments, the invention relates to the foregoing compound in non-salt form. Such compound is considered to be a “compound of the invention,” as that term is used herein.

### Salts, Compositions, Uses, Formulation, Administration and Additional Agents

### Pharmaceutically Acceptable Salts and Compositions

As discussed herein, the invention provides compounds, and pharmaceutically acceptable salts thereof, that are inhibitors of voltage-gated sodium channels, and thus the present compounds, and pharmaceutically acceptable salts thereof, are useful for the treatment of diseases, disorders, and conditions including, but not limited to chronic pain, gut pain, neuropathic pain, musculoskeletal pain, acute pain, inflammatory pain, cancer pain, idiopathic pain, postsurgical pain (e.g., bunionectomy pain, heriorrhaphy pain or abdominoplasty pain), visceral pain, multiple sclerosis, Charcot-Marie-Tooth syndrome, incontinence, pathological cough, or cardiac arrhythmia. Accordingly, in another aspect of the invention, pharmaceutical compositions are provided, wherein these compositions comprise a compound as described herein, or a pharmaceutically acceptable salt thereof, and optionally comprise a pharmaceutically acceptable carrier, adjuvant or vehicle. In certain embodiments, these compositions optionally further comprise one or more additional therapeutic agents. In some embodiments, the additional therapeutic agent is a sodium channel inhibitor.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” of a compound of this invention includes any non-toxic salt that, upon administration to a recipient, is capable of providing, either directly or indirectly, a compound of this invention or an inhibitorily active metabolite or residue thereof. The salt may be in pure form, in a mixture (e.g., solution, suspension, or colloid) with one or more other substances, or in the form of a hydrate, solvate, or co-crystal. As used herein, the term “inhibitorily active metabolite or residue thereof” means that a metabolite or residue thereof is also an inhibitor of a voltage-gated sodium channel.

Pharmaceutically acceptable salts are well known in the art. For example, S. M. Berge, et al. describe pharmaceutically acceptable salts in detail in J. Pharmaceutical Sciences, 1977, 66, 1-19, incorporated herein by reference. Pharmaceutically acceptable salts of the compound of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As described herein, the pharmaceutically acceptable compositions of the invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In another aspect, the invention features a pharmaceutical composition comprising a compound of the invention, or a pharmaceutically acceptable salt thereof, and a pharmaceutically acceptable carrier.

In another aspect, the invention features a pharmaceutical composition comprising a therapeutically effective amount of a compound, or a pharmaceutically acceptable salt thereof, and one or more pharmaceutically acceptable carriers or vehicles.

### Uses of Compounds and Pharmaceutically Acceptable Salts and Compositions

In another aspect, the invention features a method of inhibiting a voltage-gated sodium channel in a subject comprising administering to the subject a compound of the invention or a pharmaceutically acceptable salt thereof or a pharmaceutical composition thereof. In another aspect, the voltage-gated sodium channel is Na_{V}1.8.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain (e.g., bunionectomy pain, herniorrhaphy pain or abdominoplasty pain), visceral pain, multiple sclerosis, Charcot-Marie-Tooth syndrome, incontinence, pathological cough, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia comprising administering an effective amount of a compound of the invention, a pharmaceutically acceptable salt thereof or a pharmaceutical composition thereof.

In yet another aspect, the invention features a method of treating or lessening the severity in a subject of gut pain, wherein gut pain comprises inflammatory bowel disease pain, Crohn's disease pain, irritable bowel syndrome, endometriosis, polycyctic ovarian disease, salpingitis, cervicitis or interstitial cystitis pain wherein said method comprises administering an effective amount of a compound of the invention, a pharmaceutically acceptable salt thereof or a pharmaceutical composition thereof.

In yet another aspect, the invention features a method of treating or lessening the severity in a subject of neuropathic pain comprising administering an effective amount of a compound of the invention, a pharmaceutically acceptable salt thereof or a pharmaceutical composition thereof. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In yet another aspect, the invention features a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia wherein said method comprises administering an effective amount of a compound of the invention, a pharmaceutically acceptable salt thereof or a pharmaceutical composition thereof.

In yet another aspect, the invention features a method of treating or lessening the severity in a subject of musculoskeletal pain comprising administering an effective amount of a compound of the invention, a pharmaceutically acceptable salt thereof or a pharmaceutical composition thereof. In some aspects, the musculoskeletal pain comprises osteoarthritis pain.

In yet another aspect, the invention features a method of treating or lessening the severity in a subject of musculoskeletal pain, wherein musculoskeletal pain comprises osteoarthritis pain, back pain, cold pain, burn pain or dental pain wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain, ankylosing spondylitis or vulvodynia wherein said method comprises administering an effective amount of a compound of the invention, a pharmaceutically acceptable salt thereof or a pharmaceutical composition thereof.

In yet another aspect, the invention features a method of treating or lessening the severity in a subject of inflammatory pain, wherein inflammatory pain comprises rheumatoid arthritis pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises fibromyalgia pain wherein said method comprises administering an effective amount of a compound of the invention, a pharmaceutically acceptable salt thereof or a pharmaceutical composition thereof.

In yet another aspect, the invention features a method of treating or lessening the severity in a subject of idiopathic pain, wherein idiopathic pain comprises reflex sympathetic dystrophy pain, wherein said method comprises administering an effective amount of a compound of the invention, a pharmaceutically acceptable salt thereof or a pharmaceutical composition thereof.

In yet another aspect, the invention features a method of treating or lessening the severity in a subject of pathological cough wherein said method comprises administering an effective amount of a compound of the invention, a pharmaceutically acceptable salt thereof or a pharmaceutical composition thereof.

In yet another aspect, the invention features a method of treating or lessening the severity in a subject of acute pain comprising administering an effective amount of a compound of the invention, a pharmaceutically acceptable salt thereof or a pharmaceutical composition thereof. In some aspects, the acute pain comprises acute post-operative pain.

In yet another aspect, the invention features a method of treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain) comprising administering an effective amount of a compound of the invention, a pharmaceutically acceptable salt thereof or a pharmaceutical composition thereof.

In yet another aspect, the invention features a method of treating or lessening the severity in a subject of bunionectomy pain comprising administering an effective amount of a compound of the invention, a pharmaceutically acceptable salt thereof or a pharmaceutical composition thereof.

In yet another aspect, the invention features a method of treating or lessening the severity in a subject of shoulder arthroplasty pain or shoulder arthroscopy pain comprising administering an effective amount of a compound of the invention, a pharmaceutically acceptable salt thereof or a pharmaceutical composition thereof.

In yet another aspect, the invention features a method of treating or lessening the severity in a subject of herniorrhaphy pain comprising administering an effective amount of a compound of the invention, a pharmaceutically acceptable salt thereof or a pharmaceutical composition thereof.

In yet another aspect, the invention features a method of treating or lessening the severity in a subject of abdominoplasty pain comprising administering an effective amount of a compound of the invention, a pharmaceutically acceptable salt thereof or a pharmaceutical composition thereof.

In yet another aspect, the invention features a method of treating or lessening the severity in a subject of visceral pain comprising administering an effective amount of a compound of the invention, a pharmaceutically acceptable salt thereof or a pharmaceutical composition thereof. In some aspects, the visceral pain comprises visceral pain from abdominoplasty.

In yet another aspect, the invention features a method of treating or lessening the severity in a subject of a neurodegenerative disease comprising administering an effective amount of a compound of the invention, a pharmaceutically acceptable salt thereof or a pharmaceutical composition thereof. In some aspects, the neurodegenerative disease comprises multiple sclerosis. In some aspects, the neurodegenerative disease comprises Pitt Hopkins Syndrome (PTHS).

In yet another aspect, the invention features a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a method of treating or lessening the severity in a subject of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, heriorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia rheumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodiscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarthropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility, comprising administering an effective amount of a compound of the invention, a pharmaceutically acceptable salt thereof or a pharmaceutical composition thereof.

In another aspect, the invention features a method of treating or lessening the severity in a subject of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain, comprising administering an effective amount of a compound of the invention, a pharmaceutically acceptable salt thereof or a pharmaceutical composition thereof.

In another aspect, the invention features a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain, comprising administering an effective amount of a compound of the invention, a pharmaceutically acceptable salt thereof or a pharmaceutical composition thereof.

### Compounds, Pharmaceutically Acceptable Salts, and Compositions for Use

In another aspect, the invention features a compound of the invention, or a pharmaceutically acceptable salt or pharmaceutical composition thereof, for use as a medicament.

In another aspect, the invention features a compound of the invention, or a pharmaceutically acceptable salt or pharmaceutical composition thereof, for use in a method of inhibiting a voltage-gated sodium channel in a subject. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In another aspect, the invention features a compound of the invention, or a pharmaceutically acceptable salt or pharmaceutical composition thereof, for use in a method of treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In another aspect, the invention features a compound of the invention, or a pharmaceutically acceptable salt or pharmaceutical composition thereof, for use in a method of treating or lessening the severity in a subject of gut pain, wherein gut pain comprises inflammatory bowel disease pain, Crohn's disease pain, irritable bowel syndrome, endometriosis, polycyctic ovarian disease, salpingitis, cervicitis or interstitial cystitis pain.

In another aspect, the invention features a compound of the invention, or a pharmaceutically acceptable salt or pharmaceutical composition thereof, for use in a method of treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy). As used herein, the phrase “idiopathic small-fiber neuropathy” shall be understood to include any small fiber neuropathy.

In another aspect, the invention features a compound of the invention, or a pharmaceutically acceptable salt or pharmaceutical composition thereof, for use in a method of treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic cephalalgia.

In another aspect, the invention features a compound of the invention, or a pharmaceutically acceptable salt or pharmaceutical composition thereof, for use in a method of treating or lessening the severity in a subject of musculoskeletal pain. In some aspects, the musculoskeletal pain comprises osteoarthritis pain.

In another aspect, the invention features a compound of the invention, or a pharmaceutically acceptable salt or pharmaceutical composition thereof, for use in a method of treating or lessening the severity in a subject of musculoskeletal pain, wherein musculoskeletal pain comprises osteoarthritis pain, back pain, cold pain, burn pain or dental pain.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain, ankylosing spondylitis or vulvodynia.

In another aspect, the invention features a compound of the invention, or a pharmaceutically acceptable salt or pharmaceutical composition thereof, for use in a method of treating or lessening the severity in a subject of inflammatory pain, wherein inflammatory pain comprises rheumatoid arthritis pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises fibromyalgia pain.

In another aspect, the invention features a compound of the invention, or a pharmaceutically acceptable salt or pharmaceutical composition thereof, for use in a method of treating or lessening the severity in a subject of idiopathic pain, wherein idiopathic pain comprises reflex sympathetic dystrophy pain.

In another aspect, the invention features a compound of the invention, or a pharmaceutically acceptable salt or pharmaceutical composition thereof, for use in a method of treating or lessening the severity in a subject of pathological cough.

In another aspect, the invention features a compound of the invention, or a pharmaceutically acceptable salt or pharmaceutical composition thereof, for use in a method of treating or lessening the severity in a subject of acute pain. In some aspects, the acute pain comprises acute post-operative pain.

In another aspect, the invention features a compound of the invention, or a pharmaceutically acceptable salt or pharmaceutical composition thereof, for use in a method of treating or lessening the severity in a subject of postsurgical pain (e.g., joint replacement pain, soft tissue surgery pain, post-thoracotomy pain, post-mastectomy pain, hemorrhoidectomy pain, herniorrhaphy pain, bunionectomy pain or abdominoplasty pain).

In another aspect, the invention features a compound of the invention, or a pharmaceutically acceptable salt or pharmaceutical composition thereof, for use in a method of treating or lessening the severity in a subject of bunionectomy pain.

In another aspect, the invention features a compound of the invention, or a pharmaceutically acceptable salt or pharmaceutical composition thereof, for use in a method of treating or lessening the severity in a subject of shoulder arthroplasty pain or shoulder arthroscopy pain.

In another aspect, the invention features a compound of the invention, or a pharmaceutically acceptable salt or pharmaceutical composition thereof, for use in a method of treating or lessening the severity in a subject of heriorrhaphy pain.

In another aspect, the invention features a compound of the invention, or a pharmaceutically acceptable salt or pharmaceutical composition thereof, for use in a method of treating or lessening the severity in a subject of abdominoplasty pain.

In another aspect, the invention features a compound of the invention, or a pharmaceutically acceptable salt or pharmaceutical composition thereof, for use in a method of treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for use in a method of treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In another aspect, the invention features a compound of the invention, or a pharmaceutically acceptable salt or pharmaceutical composition thereof, for use in a method wherein the subject is treated with one or more additional therapeutic agents administered concurrently with, prior to, or subsequent to treatment with an effective amount of the compound, pharmaceutically acceptable salt or pharmaceutical composition. In some embodiments, the additional therapeutic agent is a sodium channel inhibitor.

In another aspect, the invention features a compound of the invention, or a pharmaceutically acceptable salt or pharmaceutical composition thereof, for use in a method of inhibiting a voltage-gated sodium channel in a biological sample comprising contacting the biological sample with an effective amount of a compound of the invention, a pharmaceutically acceptable salt thereof or a pharmaceutical composition thereof. In another aspect, the voltage-gated sodium channel is Na_{V}1.8.

In another aspect, the invention features a compound of the invention, or a pharmaceutically acceptable salt or pharmaceutical composition thereof, for use in a method of treating or lessening the severity in a subject of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia rheumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodiscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarthropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention features a compound of the invention, or a pharmaceutically acceptable salt or pharmaceutical composition thereof, for use in a method of treating or lessening the severity in a subject of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain.

In another aspect, the invention features a compound of the invention, or a pharmaceutically acceptable salt or pharmaceutical composition thereof, for use in a method of treating or lessening the severity in a subject of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Manufacture of Medicaments

In another aspect, the invention provides the use of a compound of the invention, or a pharmaceutically acceptable salt or pharmaceutical composition thereof, for the manufacture of a medicament.

In another aspect, the invention provides the use of a compound of the invention, a pharmaceutically acceptable salt thereof, or a pharmaceutical composition thereof for the manufacture of a medicament for use in inhibiting a voltage-gated sodium channel. In another aspect, the voltage-gated sodium channel is Na_{V}1.8.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain (e.g., herniorrhaphy pain, bunionectomy pain or abdominoplasty pain), visceral pain, multiple sclerosis, Charcot-Marie-Tooth syndrome, incontinence, pathological cough, or cardiac arrhythmia.

In yet another aspect, the invention provides the use of a compound of the invention, a pharmaceutically acceptable salt thereof, or a pharmaceutical composition thereof for the manufacture of a medicament for use in treating or lessening the severity in a subject of chronic pain, gut pain, neuropathic pain, musculoskeletal pain, acute pain, inflammatory pain, cancer pain, idiopathic pain, postsurgical pain, herniorrhaphy pain, bunionectomy pain, multiple sclerosis, Charcot-Marie-Tooth syndrome, incontinence, or cardiac arrhythmia.

In yet another aspect, the invention provides the use of the compound, pharmaceutically acceptable salt, or pharmaceutical composition described herein for the manufacture of a medicament for use in treating or lessening the severity in a subject of gut pain, wherein gut pain comprises inflammatory bowel disease pain, Crohn's disease pain, irritable bowel syndrome, endometriosis, polycyctic ovarian disease, salpingitis, cervicitis or interstitial cystitis pain.

In yet another aspect, the invention provides a compound of the invention, a pharmaceutically acceptable salt thereof, or a pharmaceutical composition thereof for the manufacture of a medicament for use in treating or lessening the severity in a subject of neuropathic pain. In some aspects, the neuropathic pain comprises post-herpetic neuralgia, small fiber neuropathy, diabetic neuropathy, or idiopathic small-fiber neuropathy. In some aspects, the neuropathic pain comprises diabetic neuropathy (e.g., diabetic peripheral neuropathy).

In yet another aspect, the invention provides the use of a compound of the invention, a pharmaceutically acceptable salt thereof, or a pharmaceutical composition thereof for the manufacture of a medicament for use in a treating or lessening the severity in a subject of neuropathic pain, wherein neuropathic pain comprises post-herpetic neuralgia, diabetic neuralgia, painful HIV-associated sensory neuropathy, trigeminal neuralgia, burning mouth syndrome, post-amputation pain, phantom pain, painful neuroma; traumatic neuroma; Morton's neuroma; nerve entrapment injury, spinal stenosis, carpal tunnel syndrome, radicular pain, sciatica pain; nerve avulsion injury, brachial plexus avulsion injury; complex regional pain syndrome, drug therapy induced neuralgia, cancer chemotherapy induced neuralgia, anti-retroviral therapy induced neuralgia, HIV-induced neuropathy; post spinal cord injury pain, spinal stenosis pain, small fiber neuropathy, idiopathic small-fiber neuropathy, idiopathic sensory neuropathy or trigeminal autonomic neuropathy.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain. In some aspects, the musculoskeletal pain comprises osteoarthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of musculoskeletal pain, wherein musculoskeletal pain comprises osteoarthritis pain, back pain, cold pain, burn pain or dental pain.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain, ankylosing spondylitis or vulvodynia.

In yet another aspect, the invention provides the use of a compound of the invention, a pharmaceutically acceptable salt thereof, or a pharmaceutical composition thereof for the manufacture of a medicament for use in treating or lessening the severity in a subject of inflammatory pain, wherein inflammatory pain comprises rheumatoid arthritis pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises fibromyalgia pain.

In yet another aspect, the invention provides the use of a compound of the invention, a pharmaceutically acceptable salt thereof, or a pharmaceutical composition thereof for the manufacture of a medicament for use in treating or lessening the severity in a subject of idiopathic pain, wherein idiopathic pain comprises reflex sympathetic dystrophy pain.

In yet another aspect, the invention provides the use of a compound of the invention, a pharmaceutically acceptable salt thereof, or a pharmaceutical composition thereof for the manufacture of a medicament for use in treating or lessening the severity in a subject of pathological cough.

In yet another aspect, the invention provides the use of a compound of the invention, a pharmaceutically acceptable salt thereof, or a pharmaceutical composition thereof for the manufacture of a medicament for use in treating or lessening the severity in a subject of acute pain. In some aspects, the acute pain comprises acute post-operative pain.

In yet another aspect, the invention provides the use of a compound of the invention, a pharmaceutically acceptable salt thereof, or a pharmaceutical composition thereof for the manufacture of a medicament for use in treating or lessening the severity in a subject of postsurgical pain (e.g., joint replacement pain, soft tissue surgery pain, post-thoracotomy pain, post-mastectomy pain, hemorrhoidectomy pain, heriorrhaphy pain, bunionectomy pain or abdominoplasty pain).

In yet another aspect, the invention provides the use of a compound of the invention, a pharmaceutically acceptable salt thereof, or a pharmaceutical composition thereof for the manufacture of a medicament for use in treating or lessening the severity in a subject of herniorrhaphy pain.

In yet another aspect, the invention provides the use of a compound of the invention, a pharmaceutically acceptable salt thereof, or a pharmaceutical composition thereof for the manufacture of a medicament for use in treating or lessening the severity in a subject of bunionectomy pain.

In yet another aspect, the invention provides the use of a compound of the invention, a pharmaceutically acceptable salt thereof, or a pharmaceutical composition thereof for the manufacture of a medicament for use in treating or lessening the severity in a subject of shoulder arthroplasty pain or shoulder arthroscopy pain.

In yet another aspect, the invention provides the use of a compound of the invention, a pharmaceutically acceptable salt thereof, or a pharmaceutical composition thereof for the manufacture of a medicament for use in treating or lessening the severity in a subject of abdominoplasty pain.

In yet another aspect, the invention provides the use of a compound of the invention, a pharmaceutically acceptable salt thereof, or a pharmaceutical composition thereof for the manufacture of a medicament for use in treating or lessening the severity in a subject of visceral pain. In some aspects, the visceral pain comprises visceral pain from abdominoplasty.

In another aspect, the invention features a compound of the invention, or a pharmaceutically acceptable salt or pharmaceutical composition thereof, for the manufacture of a medicament for use in treating or lessening the severity in a subject of a neurodegenerative disease. In some aspects, the neurodegenerative disease comprises multiple sclerosis. In some aspects, the neurodegenerative disease comprises Pitt Hopkins Syndrome (PTHS).

In yet another aspect, the invention provides the use of a compound of the invention, a pharmaceutically acceptable salt thereof, or a pharmaceutical composition thereof for the manufacture of a medicament for use in combination with one or more additional therapeutic agents administered concurrently with, prior to, or subsequent to treatment with the compound or pharmaceutical composition. In some embodiments, the additional therapeutic agent is a sodium channel inhibitor.

In another aspect, the invention provides the use of a compound of the invention, a pharmaceutically acceptable salt thereof, or a pharmaceutical composition thereof for the manufacture of a medicament for use in treating or lessening the severity of acute pain, sub-acute and chronic pain, nociceptive pain, neuropathic pain, inflammatory pain, nociplastic pain, arthritis, migraine, cluster headaches, tension headaches, and all other forms of headaches, trigeminal neuralgia, herpetic neuralgia, general neuralgias, epilepsy, epilepsy conditions, neurodegenerative disorders, psychiatric disorders, anxiety, depression, bipolar disorder, myotonia, arrhythmia, movement disorders, neuroendocrine disorders, ataxia, central neuropathic pain of multiple sclerosis and irritable bowel syndrome, incontinence, pathological cough, visceral pain, osteoarthritis pain, postherpetic neuralgia, diabetic neuropathy, radicular pain, sciatica, back pain, unspecific chronic back pain, head pain, neck pain, moderate pain, severe pain, intractable pain, nociceptive pain, breakthrough pain, postsurgical pain (e.g., joint replacement pain, soft tissue surgery pain, post-thoracotomy pain, post-mastectomy pain, herniorrhaphy pain, bunionectomy pain or abdominoplasty pain), cancer pain including chronic cancer pain and breakthrough cancer pain, stroke (e.g., post stroke central neuropathic pain), whiplash associated disorders, fragility fractures, spinal fractures, ankylosing spondylitis, pemphigus, Raynaud's Disease, scleroderma, systemic lupus erythematosus, Epidermolysis bullosa, gout, juvenile idiopathic arthritis, melorheostosis, polymyalgia rheumatica, pyoderma gangrenosum, chronic widespread pain, diffuse idiopathic skeletal hyperostosis, disc degeneration/herniation pain, radiculopathy, facet joint syndrome, failed back surgery syndrome, burns, carpal tunnel syndrome, Paget's disease pain, spinal canal stenosis, spondylodiscitis, transverse myelitis, Ehlers-Danlos syndrome, Fabry's disease, mastocytocytosis, neurofibromatosis, ocular neuropathic pain, sarcoidosis, spondylolysis, spondylolisthesis, chemotherapy induced oral mucositis, Charcot neuropathic osteoarthropathy, temporo-mandibular joint disorder, painful joint arthroplasties, non-cardiac chest pain, pudendal neuralgia, renal colic, biliary tract diseases, vascular leg ulcers, pain in Parkinson's disease, pain in Alzheimer's disease, cerebral ischemia, traumatic brain injury, amyotrophic lateral sclerosis, stress induced angina, exercise induced angina, palpitations, hypertension, or abnormal gastro-intestinal motility.

In another aspect, the invention provides the use of a compound of the invention, a pharmaceutically acceptable salt thereof, or a pharmaceutical composition thereof for the manufacture of a medicament for use in treating or lessening the severity of femur cancer pain; non-malignant chronic bone pain; rheumatoid arthritis; osteoarthritis; spinal stenosis; neuropathic low back pain; myofascial pain syndrome; fibromyalgia; temporomandibular joint pain; chronic visceral pain, abdominal pain; pancreatic pain; IBS pain; chronic and acute headache pain; migraine; tension headache; cluster headaches; chronic and acute neuropathic pain, post-herpetic neuralgia; diabetic neuropathy; HIV-associated neuropathy; trigeminal neuralgia; Charcot-Marie-Tooth neuropathy; hereditary sensory neuropathy; peripheral nerve injury; painful neuromas; ectopic proximal and distal discharges; radiculopathy; chemotherapy induced neuropathic pain; radiotherapy-induced neuropathic pain; persistent/chronic post-surgical pain (e.g., post amputation, post-thoracotomy, post-cardiac surgery), post-mastectomy pain; central pain; spinal cord injury pain; post-stroke pain; thalamic pain; phantom pain (e.g., following removal of lower extremity, upper extremity, breast); intractable pain; acute pain, acute post-operative pain; acute musculoskeletal pain; joint pain; mechanical low back pain; neck pain; tendonitis; injury pain; exercise pain; acute visceral pain; pyelonephritis; appendicitis; cholecystitis; intestinal obstruction; hernias; chest pain, cardiac pain; pelvic pain, renal colic pain, acute obstetric pain, labor pain; cesarean section pain; acute inflammatory pain, burn pain, trauma pain; acute intermittent pain, endometriosis; acute herpes zoster pain; sickle cell anemia; acute pancreatitis; breakthrough pain; orofacial pain; sinusitis pain; dental pain; multiple sclerosis (MS) pain; pain in depression; leprosy pain; Behcet's disease pain; adiposis dolorosa; phlebitic pain; Guillain-Barre pain; painful legs and moving toes; Haglund syndrome; erythromelalgia pain; Fabry's disease pain; bladder and urogenital disease; urinary incontinence, pathological cough; hyperactive bladder; painful bladder syndrome; interstitial cystitis (IC); prostatitis; complex regional pain syndrome (CRPS), type I, complex regional pain syndrome (CRPS) type II; widespread pain, paroxysmal extreme pain, pruritus, tinnitus, or angina-induced pain.

In another aspect, the invention provides the use of a compound of the invention, a pharmaceutically acceptable salt thereof, or a pharmaceutical composition thereof for the manufacture of a medicament for use in treating or lessening the severity of trigeminal neuralgia, migraines treated with botox, cervical radiculopathy, occipital neuralgia, axillary neuropathy, radial neuropathy, ulnar neuropathy, brachial plexopathy, thoracic radiculopathy, intercostal neuralgia, lumbosacral radiculopathy, iliolingual neuralgia, pudendal neuralgia, femoral neuropathy, meralgia paresthetica, saphenous neuropathy, sciatic neuropathy, peroneal neuropathy, tibial neuropathy, lumbosacral plexopathy, traumatic neuroma stump pain or postamputation pain.

### Administration of Compounds, Pharmaceutically Acceptable Salts, and Compositions

In certain embodiments of the invention, an “effective amount” of a compound of the invention, a pharmaceutically acceptable salt thereof, or a pharmaceutical composition thereof is that amount effective for treating or lessening the severity of one or more of the conditions recited above.

The compounds, salts, and compositions, according to the method of the invention, may be administered using any amount and any route of administration effective for treating or lessening the severity of one or more of the pain or non-pain diseases recited herein. The exact amount required will vary from subject to subject, depending on the species, age, and general condition of the subject, the severity of the condition, the particular agent, its mode of administration, and the like. The compounds, salts, and compositions of the invention are preferably formulated in dosage unit form for ease of administration and uniformity of dosage. The expression “dosage unit form” as used herein refers to a physically discrete unit of agent appropriate for the subject to be treated. It will be understood, however, that the total daily usage of the compounds, salts, and compositions of the invention will be decided by the attending physician within the scope of sound medical judgment. The specific effective dose level for any particular subject or organism will depend upon a variety of factors including the disorder being treated and the severity of the disorder; the activity of the specific compound or salt employed; the specific composition employed; the age, body weight, general health, sex and diet of the subject; the time of administration, route of administration, and rate of excretion of the specific compound or salt employed; the duration of the treatment; drugs used in combination or coincidental with the specific compound or salt employed, and like factors well known in the medical arts. The term “subject” or “patient,” as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the condition being treated. In certain embodiments, the compound, salts, and compositions of the invention may be administered orally or parenterally at dosage levels of about 0.001 mg/kg to about 1000 mg/kg, one or more times a day, effective to obtain the desired therapeutic effect.

Liquid dosage forms for oral administration include, but are not limited to, pharmaceutically acceptable emulsions, microemulsions, solutions, suspensions, syrups and elixirs. In addition to the active compound or salt, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of the compounds of the invention, it is often desirable to slow the absorption of the compounds from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compound or salt of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound or salt is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compound or salt can also be in microencapsulated form with one or more excipients as noted above. The solid dosage forms of tablets, dragees, capsules, pills, and granules can be prepared with coatings and shells such as enteric coatings, release-controlling coatings and other coatings well known in the pharmaceutical formulating art. In such solid dosage forms, the active compound or salt may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound or salt of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As described generally above, the compounds of the invention are useful as inhibitors of voltage-gated sodium channels. In one embodiment, the compounds are inhibitors of Na_{V}1.8 and thus, without wishing to be bound by any particular theory, the compounds, salts, and compositions are particularly useful for treating or lessening the severity of a disease, condition, or disorder where activation or hyperactivity of Na_{V}1.8 is implicated in the disease, condition, or disorder. When activation or hyperactivity of Na_{V}1.8 is implicated in a particular disease, condition, or disorder, the disease, condition, or disorder may also be referred to as a “Na_{V}1.8-mediated disease, condition or disorder.” Accordingly, in another aspect, the invention provides a method for treating or lessening the severity of a disease, condition, or disorder where activation or hyperactivity of Na_{V}1.8 is implicated in the disease state.

The activity of a compound utilized in this invention as an inhibitor of Na_{V}1.8 may be assayed according to methods described generally in International Publication No. WO 2014/120808 A9 and U.S. Publication No. 2014/0213616 A1, both of which are incorporated by reference in their entirety, methods described herein, and other methods known and available to one of ordinary skill in the art.

### Additional Therapeutic Agents

It will also be appreciated that the compounds, salts, and pharmaceutically acceptable compositions of the invention can be employed in combination therapies, that is, the compounds, salts, and pharmaceutically acceptable compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated.” For example, exemplary additional therapeutic agents include, but are not limited to: non-opioid analgesics (indoles such as Etodolac, Indomethacin, Sulindac, Tolmetin; naphthylalkanones such as Nabumetone; oxicams such as Piroxicam; para-aminophenol derivatives, such as Acetaminophen; propionic acids such as Fenoprofen, Flurbiprofen, Ibuprofen, Ketoprofen, Naproxen, Naproxen sodium, Oxaprozin; salicylates such as Aspirin, Choline magnesium trisalicylate, Diflunisal; fenamates such as meclofenamic acid, Mefenamic acid; and pyrazoles such as Phenylbutazone); or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infusion, neural blockade), neurosurgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Nineteenth Edition, Ed. Robert S. Porter and Justin L. Kaplan, Merck Sharp & Dohme Corp., a subsidiary of Merck & Co., Inc., 2011, and the Food and Drug Administration website, www.fda.gov, the entire contents of which are hereby incorporated by reference.

In another embodiment, additional appropriate therapeutic agents are selected from the following:
(1) an opioid analgesic, e.g. morphine, heroin, hydromorphone, oxymorphone, levorphanol, levallorphan, methadone, meperidine, fentanyl, cocaine, codeine, dihydrocodeine, oxycodone, hydrocodone, propoxyphene, nalmefene, nalorphine, naloxone, naltrexone, buprenorphine, butorphanol, nalbuphine, pentazocine, or difelikefalin;
(2) a nonsteroidal antiinflammatory drug (NSAID), e.g. aspirin, diclofenac, diflunisal, etodolac, fenbufen, fenoprofen, flufenisal, flurbiprofen, ibuprofen (including without limitation intravenous ibuprofen (e.g., Caldolor®)), indomethacin, ketoprofen, ketorolac (including without limitation ketorolac tromethamine (e.g., Toradol®)), meclofenamic acid, mefenamic acid, meloxicam, IV meloxicam (e.g., Anjeso®), nabumetone, naproxen, nimesulide, nitroflurbiprofen, olsalazine, oxaprozin, phenylbutazone, piroxicam, sulfasalazine, sulindac, tolmetin or zomepirac;
(3) a barbiturate sedative, e.g. amobarbital, aprobarbital, butabarbital, butalbital, mephobarbital, metharbital, methohexital, pentobarbital, phenobarbital, secobarbital, talbutal, thiamylal or thiopental;
(4) a benzodiazepine having a sedative action, e.g. chlordiazepoxide, clorazepate, diazepam, flurazepam, lorazepam, oxazepam, temazepam or triazolam;
(5) a histamine (H₁) antagonist having a sedative action, e.g. diphenhydramine, pyrilamine, promethazine, chlorpheniramine or chlorcyclizine;
(6) a sedative such as glutethimide, meprobamate, methaqualone or dichloralphenazone;
(7) a skeletal muscle relaxant, e.g. baclofen, carisoprodol, chlorzoxazone, cyclobenzaprine, methocarbamol or orphenadrine;
(8) an NMDA receptor antagonist, e.g. dextromethorphan ((+)-3-hydroxy-N-methylmorphinan) or its metabolite dextrorphan ((+)-3-hydroxy-N-methylmorphinan), ketamine, memantine, pyrroloquinoline quinine, cis-4-(phosphonomethyl)-2-piperidinecarboxylic acid, budipine, EN-3231 (MorphiDex®), a combination formulation of morphine and dextromethorphan), topiramate, neramexane or perzinfotel including an NR2B antagonist, e.g. ifenprodil, traxoprodil or (−)-(R)-6-{2-[4-(3-fluorophenyl)-4-hydroxy-1-piperidinyl]-1-hydroxyethyl-3,4-dihydro-2(1H)-quinolinone;
(9) an alpha-adrenergic, e.g. doxazosin, tamsulosin, clonidine, guanfacine, dexmedetomidine, modafinil, or 4-amino-6,7-dimethoxy-2-(5-methane-sulfonamido-1,2,3,4-tetrahydroisoquinolin-2-yl)-5-(2-pyridyl) quinazoline;
(10) a tricyclic antidepressant, e.g. desipramine, imipramine, amitriptyline or nortriptyline;
(11) an anticonvulsant, e.g. carbamazepine (Tegretol®), lamotrigine, topiramate, lacosamide (Vimpat®) or valproate;
(12) a tachykinin (NK) antagonist, particularly an NK-3, NK-2 or NK-1 antagonist, e.g. (alphaR,9R)-7-[3,5-bis(trifluoromethyl)benzyl]-8,9,10,11-tetrahydro-9-methyl-5-(4-methylphenyl)-7H-[1,4]diazocino[2,1-g][1,7]-naphthyridine-6-13-dione (TAK-637), 5-[[(2R,3S)-2-[(1R)-1-[3,5-bis(trifluoromethyl)phenyl]ethoxy-3-(4-fluorophenyl)-4-morpholinyl]-methyl]-1,2-dihydro-3H-1,2,4-triazol-3-one (MK-869), aprepitant, lanepitant, dapitant or 3-[[2-methoxy-5-(trifluoromethoxy)phenyl]-methylamino]-2-phenylpiperidine (2S,3S);
(13) a muscarinic antagonist, e.g oxybutynin, tolterodine, propiverine, tropsium chloride, darifenacin, solifenacin, temiverine and ipratropium;
(14) a COX-2 selective inhibitor, e.g. celecoxib, rofecoxib, parecoxib, valdecoxib, deracoxib, etoricoxib, or lumiracoxib;
(15) a coal-tar analgesic, in particular paracetamol;
(16) a neuroleptic such as droperidol, chlorpromazine, haloperidol, perphenazine, thioridazine, mesoridazine, trifluoperazine, fluphenazine, clozapine, olanzapine, risperidone, ziprasidone, quetiapine, sertindole, aripiprazole, sonepiprazole, blonanserin, iloperidone, perospirone, raclopride, zotepine, bifeprunox, asenapine, lurasidone, amisulpride, balaperidone, palindore, eplivanserin, osanetant, rimonabant, meclinertant, Miraxion® or sarizotan;
(17) a vanilloid receptor agonist (e.g. resinferatoxin or civamide) or antagonist (e.g. capsazepine, GRC-15300);
(18) a beta-adrenergic such as propranolol;
(19) a local anesthetic such as mexiletine;
(20) a corticosteroid such as dexamethasone;
(21) a 5-HT receptor agonist or antagonist, particularly a 5-HT_{1B/1D} agonist such as eletriptan, sumatriptan, naratriptan, zolmitriptan or rizatriptan;
(22) a 5-HT_{2A} receptor antagonist such as R(+)-alpha-(2,3-dimethoxy-phenyl)-1-[2-(4-fluorophenylethyl)]-4-piperidinemethanol (MDL-100907);
(23) a cholinergic (nicotinic) analgesic, such as ispronicline (TC-1734), (E)-N-methyl-4-(3-pyridinyl)-3-buten-1-amine (RJR-2403), (R)-5-(2-azetidinylmethoxy)-2-chloropyridine (ABT-594) or nicotine;
(24) Tramadol®, Tramadol ER (Ultram ER®), IV Tramadol, Tapentadol ER (Nucynta®);
(25) a PDE5 inhibitor, such as 5-[2-ethoxy-5-(4-methyl-1-piperazinyl-sulphonyl)phenyl]-1-methyl-3-n-propyl-1,6-dihydro-7H-pyrazolo[4,3-d]pyrimidin-7-one (sildenafil), (6R,12aR)-2,3,6,7,12,12a-hexahydro-2-methyl-6-(3,4-methylenedioxyphenyl)-pyrazino[2′,1′:6,1]-pyrido[3,4-b]indole-1,4-dione (IC-351 or tadalafil), 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil), 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one, 5-(5-acetyl-2-propoxy-3-pyridinyl)-3-ethyl-2-(1-isopropyl-3-azetidinyl)-2,6-dihydro-7H-pyrazolo[4,3-d]pyrimidin-7-one, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide, 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide;
(26) an alpha-2-delta ligand such as gabapentin (Neurontin®), gabapentin GR (Gralise®), gabapentin, enacarbil (Horizant®), pregabalin (Lyrica®), 3-methyl gabapentin, (1[alpha],3[alpha],5[alpha])(3-amino-methyl-bicyclo[3.2.0]hept-3-yl)-acetic acid, (3S,5R)-3-aminomethyl-5-methyl-heptanoic acid, (3S,5R)-3-amino-5-methyl-heptanoic acid, (3S,5R)-3-amino-5-methyl-octanoic acid, (2S,4S)-4-(3-chlorophenoxy)proline, (2S,4S)-4-(3-fluorobenzyl)-proline, [(1R,5R,6S)-6-(aminomethyl)bicyclo[3.2.0]hept-6-yl]acetic acid, 3-(1-aminomethyl-cyclohexylmethyl)-4H-[1,2,4]oxadiazol-5-one, C-[1-(1H-tetrazol-5-ylmethyl)-cycloheptyl]-methylamine, (3S,4S)-(1-aminomethyl-3,4-dimethyl-cyclopentyl)-acetic acid, (3S,5R)-3-aminomethyl-5-methyl-octanoic acid, (3S,5R)-3-amino-5-methyl-nonanoic acid, (3S,5R)-3-amino-5-methyl-octanoic acid, (3R,4R,5R)-3-amino-4,5-dimethyl-heptanoic acid and (3R,4R,5R)-3-amino-4,5-dimethyl-octanoic acid;
(27) a cannabinoid such as KHK-6188;
(28) metabotropic glutamate subtype 1 receptor (mGluR1) antagonist;
(29) a serotonin reuptake inhibitor such as sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine and trazodone;
(30) a noradrenaline (norepinephrine) reuptake inhibitor, such as maprotiline, lofepramine, mirtazepine, oxaprotiline, fezolamine, tomoxetine, mianserin, bupropion, bupropion metabolite hydroxybupropion, nomifensine and viloxazine (Vivalan®), especially a selective noradrenaline reuptake inhibitor such as reboxetine, in particular (S,S)-reboxetine;
(31) a dual serotonin-noradrenaline reuptake inhibitor, such as venlafaxine, venlafaxine metabolite O-desmethylvenlafaxine, clomipramine, clomipramine metabolite desmethylclomipramine, duloxetine (Cymbalta®), milnacipran and imipramine;
(32) an inducible nitric oxide synthase (iNOS) inhibitor such as S-[2-[(1-iminoethyl)amino]ethyl]-L-homocysteine, S-[2-[(1-iminoethyl)-amino]ethyl]-4,4-dioxo-L-cysteine, S-[2-[(1-iminoethyl)amino]ethyl]-2-methyl-L-cysteine, (2S,5Z)-2-amino-2-methyl-7-[(1-iminoethyl)amino]-5-heptenoic acid, 2-[[(1R,3S)-3-amino-4-hydroxy-1-(5-thiazolyl)-butyl]thio]-S-chloro-S-pyridinecarbonitrile; 2-[[(1R,3S)-3-amino-4-hydroxy-1-(5-thiazolyl)butyl]thio]-4-chlorobenzonitrile, (2S,4R)-2-amino-4-[[2-chloro-5-(trifluoromethyl)phenyl]thio]-5-thiazolebutanol, 2-[[(1R,3S)-3-amino-4-hydroxy-1-(5-thiazolyl) butyl]thio]-6-(trifluoromethyl)-3-pyridinecarbonitrile, 2-[[(1R,3S)-3-amino-4-hydroxy-1-(5-thiazolyl)butyl]thio]-5-chlorobenzonitrile, N-[4-[2-(3-chlorobenzylamino)ethyl]phenyl]thiophene-2-carboxamidine, NXN-462, or guanidinoethyldisulfide;
(33) an acetylcholinesterase inhibitor such as donepezil;
(34) a prostaglandin E2 subtype 4 (EP4) antagonist such as N-[({2-[4-(2-ethyl-4,6-dimethyl-1H-imidazo[4,5-c]pyridin-1-yl)phenyl]ethyl}amino)-carbonyl]-4-methylbenzenesulfonamide or 4-[(15)-1-({[5-chloro-2-(3-fluorophenoxy)pyridin-3-yl]carbonyl}amino)ethyl]benzoic acid;
(35) a leukotriene B4 antagonist; such as 1-(3-biphenyl-4-ylmethyl-4-hydroxy-chroman-7-yl)-cyclopentanecarboxylic acid (CP-105696), 5-[2-(2-Carboxyethyl)-3-[6-(4-methoxyphenyl)-5E-hexenyl]oxyphenoxy]-valeric acid (ONO-4057) or DPC-11870;
(36) a 5-lipoxygenase inhibitor, such as zileuton, 6-[(3-fluoro-5-[4-methoxy-3,4,5,6-tetrahydro-2H-pyran-4-yl])phenoxy-methyl]-1-methyl-2-quinolone (ZD-2138), or 2,3,5-trimethyl-6-(3-pyridylmethyl)-1,4-benzoquinone (CV-6504);
(37) a sodium channel blocker, such as lidocaine, lidocaine plus tetracaine cream (ZRS-201) or eslicarbazepine acetate;
(38) aNa_{V}1.7 blocker, such as XEN-402, XEN403, TV-45070, PF-05089771, CNV1014802, GDC-0276, RG7893 BIIB-074 (Vixotrigine), BIIB-095, ASP-1807, DSP-3905, OLP-1002, RQ-00432979, FX-301, DWP-1706, DWP-17061, IMB-110, IMB-111, IMB-112 and such as those disclosed in WO2011/140425 (US2011/306607); WO2012/106499 (US2012196869); WO2012/112743 (US2012245136); WO2012/125613 (US2012264749), WO2012/116440 (US2014187533), WO2011026240 (US2012220605), U.S. Pat. Nos. 8,883,840, 8,466,188, WO2013/109521 (US2015005304), CN111217776, WO2020/117626, WO2021/252822, WO2021/252818, WO2021/252820, WO2014/201173, WO2012/125973, WO2013/086229, WO2013/134518, WO2014/201206, or WO2016/141035 the entire contents of each application hereby incorporated by reference;
(38a) a Na_{V}1.7 blocker such as (2-benzylspiro[3,4-dihydropyrrolo[1,2-a]pyrazine-1,4′-piperidine]-1′-yl)-(4-isopropoxy-3-methyl-phenyl)methanone, 2,2,2-trifluoro-1-[1′-[3-methoxy-4-[2-(trifluoromethoxy)ethoxy]benzoyl]-2,4-dimethyl-spiro[3,4-dihydropyrrolo[1,2-a]pyrazine-1,4′-piperidine]-6-yl]ethanone, [8-fluoro-2-methyl-6-(trifluoromethyl)spiro[3,4-dihydropyrrolo[1,2-a]pyrazine-1,4′-piperidine]-1′-yl]-(4-isobutoxy-3-methoxy-phenyl)methanone, 1-(4-benzhydrylpiperazin-1-yl)-3-[2-(3,4-dimethylphenoxy)ethoxy]propan-2-ol, (4-butoxy-3-methoxy-phenyl)-[2-methyl-6-(trifluoromethyl)spiro[3,4-dihydropyrrolo[1,2-a]pyrazine-1,4′-piperidine]-1′-yl]methanone, [8-fluoro-2-methyl-6-(trifluoromethyl)spiro[3,4-dihydropyrrolo[1,2-a]pyrazine-1,4′-piperidine]-1′-yl]-(5-isopropoxy-6-methyl-2-pyridyl)methanone, (4-isopropoxy-3-methyl-phenyl)-[2-methyl-6-(1,1,2,2,2-pentafluoroethyl)spiro[3,4-dihydropyrrolo[1,2-a]pyrazine-1,4′-piperidine]-1′-yl]methanone, 5-[2-methyl-4-[2-methyl-6-(2,2,2-trifluoroacetyl)spiro[3,4-dihydropyrrolo[1,2-a]pyrazine-1,4′-piperidine]-1′-carbonyl]phenyl]pyridine-2-carbonitrile, (4-isopropoxy-3-methyl-phenyl)-[6-(trifluoromethyl)spiro[3,4-dihydro-2H-pyrrolo[1,2-a]pyrazine-1,4′-piperidine]-1′-yl]methanone, 2,2,2-trifluoro-1-[1′-[3-methoxy-4-[2-(trifluoromethoxy)ethoxy]benzoyl]-2-methyl-spiro[3,4-dihydropyrrolo[1,2-a]pyrazine-1,4′-piperidine]-6-yl]ethanone, 2,2,2-trifluoro-1-[1′-(5-isopropoxy-6-methyl-pyridine-2-carbonyl)-3,3-dimethyl-spiro[2,4-dihydropyrrolo[1,2-a]pyrazine-1,4′-piperidine]-6-yl]ethanone, 2,2,2-trifluoro-1-[1′-(5-isopentyloxypyridine-2-carbonyl)-2-methyl-spiro[3,4-dihydropyrrolo[1,2-a]pyrazine-1,4′-piperidine]-6-yl]ethanone, (4-isopropoxy-3-methoxy-phenyl)-[2-methyl-6-(trifluoromethyl)spiro[3,4-dihydropyrrolo[1,2-a]pyrazine-1,4′-piperidine]-1′-yl]methanone, 2,2,2-trifluoro-1-[1′-(5-isopentyloxypyridine-2-carbonyl)-2,4-dimethyl-spiro[3,4-dihydropyrrolo[1,2-a]pyrazine-1,4′-piperidine]-6-yl]ethanone, 1-[(3S)-2,3-dimethyl-1′-[4-(3,3,3-trifluoropropoxymethyl)benzoyl]spiro[3,4-dihydropyrrolo[1,2-a]pyrazine-1,4′-piperidine]-6-yl]-2,2,2-trifluoro-ethanone, [8-fluoro-2-methyl-6-(trifluoromethyl)spiro[3,4-dihydropyrrolo[1,2-a]pyrazine-1,4′-piperidine]-1′-yl]-[3-methoxy-4-[(1R)-1-methylpropoxy]phenyl]methanone, 2,2,2-trifluoro-1-[1′-(5-isopropoxy-6-methyl-pyridine-2-carbonyl)-2,4-dimethyl-spiro[3,4-dihydropyrrolo[1,2-a]pyrazine-1,4′-piperidine]-6-yl]ethanone, 1-[1′-[4-methoxy-3-(trifluoromethyl)benzoyl]-2-methyl-spiro[3,4-dihydropyrrolo[1,2-a]pyrazine-1,4′-piperidine]-6-yl]-2,2-dimethyl-propan-1-one, (4-isopropoxy-3-methyl-phenyl)-[2-methyl-6-(trifluoromethyl)spiro[3,4-dihydropyrrolo[1,2-a]pyrazine-1,4′-piperidine]-1′-yl]methanone, [2-methyl-6-(1-methylcyclopropanecarbonyl)spiro[3,4-dihydropyrrolo[1,2-a]pyrazine-1,4′-piperidine]-1′-yl]-[4-(3,3,3-trifluoropropoxymethyl)phenyl]methanone, 4-bromo-N-(4-bromophenyl)-3-[(1-methyl-2-oxo-4-piperidyl)sulfamoyl]benzamide or (3-chloro-4-isopropoxy-phenyl)-[2-methyl-6-(1,1,2,2,2-pentafluoroethyl)spiro[3,4-dihydropyrrolo[1,2-a]pyrazine-1,4′-piperidine]-1′-yl]methanone.
(39) a Na_{V}1.8 blocker, such as PF-04531083, PF-06372865 and such as those disclosed in WO2008/135826 (US2009048306), WO2006/011050 (US2008312235), WO2013/061205 (US2014296313), US20130303535, WO2013131018, U.S. Pat. No. 8,466,188, WO2013114250 (US2013274243), WO2014/120808 (US2014213616), WO2014/120815 (US2014228371) WO2014/120820 (US2014221435), WO2015/010065 (US20160152561), WO2015/089361 (US20150166589), WO2019/014352 (US20190016671), WO2018/213426, WO2020/146682, WO2020/146612, WO2020/014243, WO2020/014246, WO2020/092187, WO2020/092667 (US2020140411), WO2020/144375, WO2020/261114, WO2020/140959, WO2020/151728, WO2021/032074, WO2021/047622 (CN112479996), WO2021/257490, WO/2021/257420, WO2021/257418, WO2022/263498, WO2022/235558, WO2022/235859, CN112390745, CN111808019, CN112225695, CN112457294, CN112300051, CN112300069, CN112441969, and CN114591293, the entire contents of each application hereby incorporated by reference;
(39a) a Na_{V}1.8 blocker such as 4,5-dichloro-2-(4-fluoro-2-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-4-(perfluoroethyl)benzamide, 4,5-dichloro-2-(4-fluorophenoxy)-N-(2-oxo-1,2-dihydropyridin-4-yl)benzamide, 4,5-dichloro-2-(3-fluoro-4-methoxyphenoxy)-N-(2-oxo-1,2-dihydropyridin-4-yl)benzamide, 2-(4-fluoro-2-methoxyphenoxy)-N-(2-oxo-1,2-dihydropyridin-4-yl)-5-(trifluoromethyl)benzamide, N-(2-oxo-1,2-dihydropyridin-4-yl)-2-(4-(trifluoromethoxy)phenoxy)-4-(trifluoromethyl)benzamide, 2-(4-fluorophenoxy)-N-(2-oxo-1,2-dihydropyridin-4-yl)-4-(perfluoroethyl)benzamide, 5-chloro-2-(4-fluoro-2-methoxyphenoxy)-N-(2-oxo-1,2-dihydropyridin-4-yl)benzamide, N-(2-oxo-1,2-dihydropyridin-4-yl)-2-(4-(trifluoromethoxy)phenoxy)-5-(trifluoromethyl)benzamide, 2-(4-fluoro-2-methylphenoxy)-N-(2-oxo-1,2-dihydropyridin-4-yl)-5-(trifluoromethyl)benzamide, 2-(2-chloro-4-fluorophenoxy)-N-(2-oxo-1,2-dihydropyridin-4-yl)-5-(trifluoromethyl)benzamide, 5-chloro-2-(4-fluoro-2-methylphenoxy)-N-(2-oxo-1,2-dihydropyridin-4-yl)benzamide, 4-chloro-2-(4-fluoro-2-methylphenoxy)-N-(2-oxo-1,2-dihydropyridin-4-yl)benzamide, 5-chloro-2-(2-chloro-4-fluorophenoxy)-N-(2-oxo-1,2-dihydropyridin-4-yl)benzamide, 2-((5-fluoro-2-hydroxybenzyl)oxy)-N-(2-oxo-1,2-dihydropyridin-4-yl)-4-(trifluoromethyl)benzamide, N-(2-oxo-1,2-dihydropyridin-4-yl)-2-(o-tolyloxy)-5-(trifluoromethyl)benzamide, 2-(2,4-difluorophenoxy)-N-(2-oxo-1,2-dihydropyridin-4-yl)-4-(trifluoromethyl)benzamide, N-(2-oxo-1,2-dihydropyridin-4-yl)-2-(2-(trifluoromethoxy)phenoxy)-5-(trifluoromethyl)benzamide, 2-(4-fluorophenoxy)-N-(2-oxo-1,2-dihydropyridin-4-yl)-5-(trifluoromethyl)benzamide, 2-(4-fluoro-2-methyl-phenoxy)-N-(2-oxo-1H-pyridin-4-yl)-4-(trifluoromethyl)benzamide, [4-[[2-(4-fluoro-2-methyl-phenoxy)-4-(trifluoromethyl)benzoyl]amino]-2-oxo-1-pyridyl]methyl dihydrogen phosphate, 2-(4-fluoro-2-(methyl-d₃)phenoxy)-N-(2-oxo-1,2-dihydropyridin-4-yl)-4-(trifluoromethyl)benzamide, (4-(2-(4-fluoro-2-(methyl-d₃)phenoxy)-4-(trifluoromethyl)benzamido)-2-oxopyridin-1(2H)-yl)methyl dihydrogen phosphate, 3-(4-fluoro-2-methoxyphenoxy)-N-(3-(methylsulfonyl)phenyl)quinoxaline-2-carboxamide, 3-(2-chloro-4-fluorophenoxy)-N-(3-sulfamoylphenyl)quinoxaline-2-carboxamide, 3-(2-chloro-4-methoxyphenoxy)-N-(3-sulfamoylphenyl)quinoxaline-2-carboxamide, 3-(4-chloro-2-methoxyphenoxy)-N-(3-sulfamoylphenyl)quinoxaline-2-carboxamide, 4-(3-(4-(trifluoromethoxy)phenoxy)quinoxaline-2-carboxamido)picolinic acid, 2-(2,4-difluorophenoxy)-N-(3-sulfamoylphenyl)quinoline-3-carboxamide, 2-(4-fluoro-2-methoxyphenoxy)-N-(3-sulfamoylphenyl)quinoline-3-carboxamide, 3-(2,4-difluorophenoxy)-N-(3-sulfamoylphenyl)quinoxaline-2-carboxamide, N-(3-sulfamoylphenyl)-2-(4-(trifluoromethoxy)phenoxy)quinoline-3-carboxamide, N-(3-sulfamoylphenyl)-3-(4-(trifluoromethoxy)phenoxy)quinoxaline-2-carboxamide, 3-(4-chloro-2-methylphenoxy)-N-(3-sulfamoylphenyl)quinoxaline-2-carboxamide, 5-(3-(4-(trifluoromethoxy)phenoxy)quinoxaline-2-carboxamido)picolinic acid, 3-(4-fluoro-2-methoxyphenoxy)-N-(2-oxo-2,3-dihydro-1H-benzo[d]imidazol-5-yl)quinoxaline-2-carboxamide, 3-(4-fluoro-2-methoxyphenoxy)-N-(pyridin-4-yl)quinoxaline-2-carboxamide, 3-(4-fluorophenoxy)-N-(3-sulfamoylphenyl)quinoxaline-2-carboxamide, N-(3-cyanophenyl)-3-(4-fluoro-2-methoxyphenoxy)quinoxaline-2-carboxamide, N-(4-carbamoylphenyl)-3-(4-fluoro-2-methoxyphenoxy)quinoxaline-2-carboxamide, 4-(3-(4-(trifluoromethoxy)phenoxy)quinoxaline-2-carboxamido)benzoic acid, N-(4-cyanophenyl)-3-(4-fluoro-2-methoxyphenoxy)quinoxaline-2-carboxamide, 5-(4,5-dichloro-2-(4-fluoro-2-methoxyphenoxy)benzamido)picolinic acid, 5-(2-(2,4-dimethoxyphenoxy)-4,6-bis(trifluoromethyl)benzamido)picolinic acid, 4-(4,5-dichloro-2-(4-fluoro-2-methoxyphenoxy)benzamido)benzoic acid, 5-(2-(4-fluoro-2-methoxyphenoxy)-4,6-bis(trifluoromethyl)benzamido)picolinic acid, 4-(2-(4-fluoro-2-methoxyphenoxy)-4-(perfluoroethyl)benzamido)benzoic acid, 5-(2-(4-fluoro-2-methoxyphenoxy)-4-(perfluoroethyl)benzamido)picolinic acid, 4-(2-(4-fluoro-2-methylphenoxy)-4-(trifluoromethyl)benzamido)benzoic acid, 5-(4,5-dichloro-2-(4-fluoro-2-methoxyphenoxy)benzamido)picolinic acid, 4-(2-(2-chloro-4-fluorophenoxy)-4-(perfluoroethyl)benzamido)benzoic acid, 4-(2-(4-fluoro-2-methylphenoxy)-4-(perfluoroethyl)benzamido)benzoic acid, 4-(4,5-dichloro-2-(4-(trifluoromethoxy)phenoxy)benzamido)benzoic acid, 4-(4,5-dichloro-2-(4-chloro-2-methylphenoxy)benzamido)benzoic acid, 5-(4-(tert-butyl)-2-(4-fluoro-2-methoxyphenoxy)benzamido)picolinic acid, 5-(4,5-dichloro-2-(4-(trifluoromethoxy)phenoxy)benzamido)picolinic acid, 4-(4,5-dichloro-2-(4-fluoro-2-methylphenoxy)benzamido)benzoic acid, 5-(4,5-dichloro-2-(2,4-dimethoxyphenoxy)benzamido)picolinic acid, 5-(4,5-dichloro-2-(2-chloro-4-fluorophenoxy)benzamido)picolinic acid, 5-(4,5-dichloro-2-(4-fluoro-2-methylphenoxy)benzamido)picolinic acid, 4-(4,5-dichloro-2-(4-chloro-2-methoxyphenoxy)benzamido)benzoic acid, 5-(4,5-dichloro-2-(2,4-difluorophenoxy)benzamido)picolinic acid, 2-(4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-5-(trifluoromethyl)benzamide, 2-(4-fluorophenoxy)-N-(3-sulfamoylphenyl)-4-(trifluoromethyl)benzamide, 2-(2-chloro-4-fluorophenoxy)-N-(3-sulfamoylphenyl)-6-(trifluoromethyl)benzamide, 2-(2-chloro-4-fluorophenoxy)-5-(difluoromethyl)-N-(3-sulfamoylphenyl)benzamide, 2-(4-fluorophenoxy)-4-(perfluoroethyl)-N-(3-sulfamoylphenyl)benzamide, 2-(4-chloro-2-methoxyphenoxy)-4-(perfluoroethyl)-N-(3-sulfamoylphenyl)benzamide, 2-(4-fluoro-2-methoxyphenoxy)-N-(3-sulfamoylphenyl)-5-(trifluoromethyl)benzamide, 5-chloro-2-(4-fluoro-2-methylphenoxy)-N-(3-sulfamoylphenyl)benzamide, 4,5-dichloro-2-(4-fluoro-2-methoxyphenoxy)-N-(3-sulfamoylphenyl)benzamide, 2,4-dichloro-6-(4-chloro-2-methoxyphenoxy)-N-(3-sulfamoylphenyl)benzamide, 2,4-dichloro-6-(4-fluoro-2-methylphenoxy)-N-(3-sulfamoylphenyl)benzamide, 2-(4-fluoro-2-methoxyphenoxy)-N-(3-sulfamoylphenyl)-4,6-bis(trifluoromethyl)benzamide, 2-(4-fluoro-2-methylphenoxy)-N-(3-sulfamoylphenyl)-4,6-bis(trifluoromethyl)benzamide, 5-chloro-2-(2-chloro-4-fluorophenoxy)-N-(3-sulfamoylphenyl)benzamide, 2-(4-fluoro-2-methoxyphenoxy)-N-(3-sulfamoylphenyl)-4-(trifluoromethoxy)benzamide, 2-(4-fluoro-2-methoxyphenoxy)-N-(3-sulfamoylphenyl)-4-(trifluoromethyl)benzamide, 4,5-dichloro-2-(4-fluorophenoxy)-N-(3-sulfamoylphenyl)benzamide, 2-(4-fluoro-2-methoxyphenoxy)-4-(perfluoroethyl)-N-(3-sulfamoylphenyl)benzamide, 5-fluoro-2-(4-fluoro-2-methylphenoxy)-N-(3-sulfamoylphenyl)benzamide, 2-(2-chloro-4-fluorophenoxy)-4-cyano-N-(3-sulfamoylphenyl)benzamide, N-(3-sulfamoylphenyl)-2-(4-(trifluoromethoxy)phenoxy)-4-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethyl)benzamide, N-(3-carbamoyl-4-fluoro-phenyl)-2-fluoro-6-[2-methoxy-4-(trifluoromethoxy)phenoxy]-3-(trifluoromethyl)benzamide, N-(3-carbamoyl-4-fluoro-phenyl)-2-fluoro-6-[2-(trideuteriomethoxy)-4-(trifluoromethoxy)phenoxy]-3-(trifluoromethoxy)benzamide, 4-[[2-fluoro-6-[2-methoxy-4-(trifluoromethoxy)phenoxy]-3-(trifluoromethyl)benzoyl]amino]pyridine-2-carboxamide, 4-[[3-chloro-2-fluoro-6-[2-methoxy-4-(trifluoromethoxy)phenoxy]benzoyl]amino]pyridine-2-carboxamide, 4-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-3-(difluoromethyl)-2-fluoro-6-[2-methoxy-4-(trifluoromethoxy)phenoxy]benzamide, 4-[[2-fluoro-6-[2-(trideuteriomethoxy)-4-(trifluoromethoxy)phenoxy]-3-(trifluoromethoxy)benzoyl]amino]pyridine-2-carboxamide, N-(3-carbamoyl-4-fluoro-phenyl)-6-[2-chloro-4-(trifluoromethoxy)phenoxy]-2-fluoro-3-(trifluoromethyl)benzamide, N-(3-carbamoyl-4-fluoro-phenyl)-2-fluoro-6-[2-methyl-4-(trifluoromethoxy)phenoxy]-3-(trifluoromethyl)benzamide, N-(3-carbamoyl-4-fluoro-phenyl)-2,3,4-trifluoro-6-[2-methoxy-4-(trifluoromethoxy)phenoxy]benzamide, N-(2-carbamoyl-4-pyridyl)-3-fluoro-5-[2-methoxy-4-(trifluoromethoxy)phenoxy]-2-(trifluoromethyl)pyridine-4-carboxamide, 4-[[6-[2-(difluoromethoxy)-4-(trifluoromethoxy)phenoxy]-2-fluoro-3-(trifluoromethyl)benzoyl]amino]pyridine-2-carboxamide, N-(3-carbamoyl-4-fluoro-phenyl)-6-[3-chloro-4-(trifluoromethoxy)phenoxy]-2-fluoro-3-(trifluoromethyl)benzamide, N-(3-carbamoyl-4-fluoro-phenyl)-2-fluoro-6-[4-(trifluoromethoxy)phenoxy]-3-(trifluoromethyl)benzamide, N-(4-carbamoyl-3-fluoro-phenyl)-2-fluoro-6-[2-methoxy-4-(trifluoromethoxy)phenoxy]-3-(trifluoromethyl)benzamide, 4-[[2-fluoro-6-[2-(trideuteriomethoxy)-4-(trifluoromethoxy)phenoxy]-4-(trifluoromethyl)benzoyl]amino]pyridine-2-carboxamide, N-(3-carbamoyl-4-fluoro-phenyl)-2-fluoro-6-[3-fluoro-4-(trifluoromethoxy)phenoxy]-3-(trifluoromethyl)benzamide, N-(3-carbamoyl-4-fluoro-phenyl)-2-[2-methoxy-4-(trifluoromethoxy)phenoxy]-5-(1,1,2,2,2-pentafluoroethyl)benzamide, 4-[[4-(difluoromethoxy)-2-fluoro-6-[2-methoxy-4-(trifluoromethoxy)phenoxy]benzoyl]amino]pyridine-2-carboxamide, N-(3-carbamoyl-4-fluoro-phenyl)-2-fluoro-6-[2-fluoro-4-(trifluoromethoxy)phenoxy]-3-(trifluoromethyl)benzamide, 4-[[4-cyclopropyl-2-fluoro-6-[2-methoxy-4-(trifluoromethoxy)phenoxy]benzoyl]amino]pyridine-2-carboxamide, N-(3-carbamoyl-4-fluoro-phenyl)-5-fluoro-2-[2-methoxy-4-(trifluoromethoxy)phenoxy]-4-(trifluoromethyl)benzamide, 5-[[2-fluoro-6-[2-(trideuteriomethoxy)-4-(trifluoromethoxy)phenoxy]-3-(trifluoromethyl)benzoyl]amino]pyridine-2-carboxamide, N-(3-carbamoyl-4-fluoro-phenyl)-2-fluoro-6-(4-fluorophenoxy)-3-(trifluoromethyl)benzamide, or 4-[[2-fluoro-6-[3-fluoro-2-methoxy-4-(trifluoromethoxy)phenoxy]-3-(trifluoromethyl)benzoyl]amino]pyridine-2-carboxamide;
(40) a combined Na_{V}1.7 and Na_{V}1.8 blocker, such as DSP-2230, Lohocla201 or BL-1021;
(41) a 5-HT3 antagonist, such as ondansetron;
(42) a TPRV 1 receptor agonist, such as capsaicin (NeurogesX®, Qutenza®); and the pharmaceutically acceptable salts and solvates thereof,
(43) a nicotinic receptor antagonist, such as varenicline;
(44) an N-type calcium channel antagonist, such as Z-160;
(45) a nerve growth factor antagonist, such as tanezumab;
(46) an endopeptidase stimulant, such as senrebotase;
(47) an angiotensin II antagonist, such as EMA-401;
(48) acetaminophen (including without limitation intravenous acetaminophen (e.g., Ofirmev®));
(49) bupivacaine (including without limitation bupivacaine liposome injectable suspension (e.g., Exparel®) bupivacaine ER (Posimir), bupivacaine collagen (Xaracoll) and transdermal bupivacaine (Eladur®)); and
(50) bupivacaine and meloxicam combination (e.g., HTX-011).

In one embodiment, the additional appropriate therapeutic agents are selected from V-116517, Pregabalin, controlled release Pregabalin, Ezogabine (Potiga®). Ketamine/amitriptyline topical cream (Amiket®), AVP-923, Perampanel (E-2007), Ralfinamide, transdermal bupivacaine (Eladur®), CNV1014802, JNJ-10234094 (Carisbamate), BMS-954561 or ARC-4558.

In another embodiment, the additional appropriate therapeutic agents are selected from N-(6-amino-5-(2,3,5-trichlorophenyl)pyridin-2-yl)acetamide; N-(6-amino-5-(2-chloro-5-methoxyphenyl)pyridin-2-yl)-1-methyl-1H-pyrazole-5-carboxamide; or 3-((4-(4-(trifluoromethoxy)phenyl)-1H-imidazol-2-yl)methyl)oxetan-3-amine.

In another embodiment, the additional therapeutic agent is selected from a GlyT2/5HT2 inhibitor, such as Operanserin (VVZ149), a TRPV modulator such as CA008, CMX-020, NE06860, FTABS, CNTX4975, MCP101, MDR16523, or MDR652, a EGRI inhibitor such as Brivoglide (AYX1), an NGF inhibitor such as Tanezumab, Fasinumab, ASP6294, MEDI7352, a Mu opioid agonist such as Cebranopadol, NKTR181 (oxycodegol), a CB-1 agonist such as NEO1940 (AZN1940), an imidazoline 12 agonist such as CR4056 or a p75NTR-Fc modulator such as LEVI-04.

In another embodiment, the additional therapeutic agent is oliceridine or ropivacaine (TLC590).

In another embodiment, the additional therapeutic agent is a Na_{V}1.7 blocker such as ST-2427, ST-2578 and those disclosed in WO2010/129864, WO2015/157559, WO2017/059385, WO2018/183781, WO2018/183782, WO2020/072835, and/or WO2022/036297 the entire contents of each application hereby incorporated by reference.

In another embodiment, the additional therapeutic agent is ASP18071, CC-8464, ANP-230, ANP-231, NOC-100, NTX-1175, ASN008, NW3509, AM-6120, AM-8145, AM-0422, BL-017881, NTM-006, Opiranserin (Unafrar^{m}), brivoligide, SR419, NRD.E1, LX9211, LY3016859, isC-17536, NFX-88, LAT-8881, AP-235, NYX 2925, CNTX-6016, S-600918, S-637880, RQ-00434739, KLS-2031, MEDI 7352, or XT-150.

In another embodiment, the additional therapeutic agent is Olinvyk, Zynrelef, Seglentis, Neumentum, Nevakar, HTX-034, CPL-01, ACP-044, HRS-4800, Tarlige, BAY2395840, LY3526318, Eliapixant, TRV045, RTA901, NRD1355-E1, MT-8554, LY3556050, AP-325, tetrodotoxin, Otenaproxesul, CFTX-1554, Funapide, iN1011-N17, JMKX000623/ODM-111, ETX-801, OLP-1002, ANP-230/DSP-2230, iN1011-N17, DSP-3905 or ACD440.

In another embodiment, the additional therapeutic agent is a sodium channel inhibitor (also known as a sodium channel blocker), such as the Na_{V}1.7 and Na_{V}1.8 blockers identified above.

The amount of additional therapeutic agent present in the compositions of this invention may be no more than the amount that would normally be administered in a composition comprising that therapeutic agent as the only active agent. The amount of additional therapeutic agent in the presently disclosed compositions may range from about 10% to 100% of the amount normally present in a composition comprising that agent as the only therapeutically active agent.

The compounds and salts of this invention or pharmaceutically acceptable compositions thereof may also be incorporated into compositions for coating an implantable medical device, such as prostheses, artificial valves, vascular grafts, stents and catheters. Accordingly, the invention, in another aspect, includes a composition for coating an implantable device comprising a compound or salt of the invention as described generally above, and in classes and subclasses herein, and a carrier suitable for coating said implantable device. In still another aspect, the invention includes an implantable device coated with a composition comprising a compound or salt of the invention as described generally above, and in classes and subclasses herein, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

Another aspect of the invention relates to inhibiting Na_{V}1.8 activity in a biological sample or a subject, which method comprises administering to the subject, or contacting said biological sample with a compound of the invention, a pharmaceutically acceptable salt thereof, or a pharmaceutical composition thereof. The term “biological sample,” as used herein, includes, without limitation, cell cultures or extracts thereof, biopsied material obtained from a mammal or extracts thereof, and blood, saliva, urine, feces, semen, tears, or other body fluids or extracts thereof.

Inhibition of Na_{V}1.8 activity in a biological sample is useful for a variety of purposes that are known to one of skill in the art. Examples of such purposes include, but are not limited to, the study of sodium channels in biological and pathological phenomena; and the comparative evaluation of new sodium channel inhibitors.

### Synthesis of the Compounds of the Invention

The compounds of the invention can be prepared from known materials by the methods described in the Examples, other similar methods, and other methods known to one skilled in the art. As one skilled in the art would appreciate, the functional groups of the intermediate compounds in the methods described below may need to be protected by suitable protecting groups. Protecting groups may be added or removed in accordance with standard techniques, which are well-known to those skilled in the art. The use of protecting groups is described in detail in T. G. M. Wuts et al., Greene's Protective Groups in Organic Synthesis (4th ed. 2006).

### Radiolabeled Analogs of the Compounds of the Invention

In another aspect, the invention relates to radiolabeled analogs of the compounds of the invention. As used herein, the term “radiolabeled analogs of the compounds of the invention” refers to compounds that are identical to the compounds of the invention, as described herein, including all embodiments thereof, except that one or more atoms has been replaced with a radioisotope of the atom present in the compounds of the invention.

As used herein, the term “radioisotope” refers to an isotope of an element that is known to undergo spontaneous radioactive decay. Examples of radioisotopes include ³H, ¹⁴C, ³²P ³⁵S, ¹⁸F, ³⁶Cl, and the like, as well as the isotopes for which a decay mode is identified in V. S. Shirley & C. M. Lederer, Isotopes Project, Nuclear Science Division, Lawrence Berkeley Laboratory, Table of Nuclides (January 1980).

The radiolabeled analogs can be used in a number of beneficial ways, including in various types of assays, such as substrate tissue distribution assays. For example, tritium (³H)- and/or carbon-14 (¹⁴C)-labeled compounds may be useful for various types of assays, such as substrate tissue distribution assays, due to relatively simple preparation and excellent detectability.

In another aspect, the invention relates to pharmaceutically acceptable salts of the radiolabeled analogs, in accordance with any of the embodiments described herein in connection with the compounds of the invention.

In another aspect, the invention relates to pharmaceutical compositions comprising the radiolabeled analogs, or pharmaceutically acceptable salts thereof, and a pharmaceutically acceptable carrier, adjuvant or vehicle, in accordance with any of the embodiments described herein in connection with the compounds of the invention.

In another aspect, the invention relates to methods of inhibiting voltage-gated sodium channels and methods of treating or lessening the severity of various diseases and disorders, including pain, in a subject comprising administering an effective amount of the radiolabeled analogs, pharmaceutically acceptable salts thereof, and pharmaceutical compositions thereof, in accordance with any of the embodiments described herein in connection with the compounds of the invention.

In another aspect, the invention relates to radiolabeled analogs, pharmaceutically acceptable salts thereof, and pharmaceutical compositions thereof, for use, in accordance with any of the embodiments described herein in connection with the compounds of the invention.

In another aspect, the invention relates to the use of the radiolabeled analogs, or pharmaceutically acceptable salts thereof, and pharmaceutical compositions thereof, for the manufacture of medicaments, in accordance with any of the embodiments described herein in connection with the compounds of the invention.

In another aspect, the radiolabeled analogs, pharmaceutically acceptable salts thereof, and pharmaceutical compositions thereof, can be employed in combination therapies, in accordance with any of the embodiments described herein in connection with the compounds of the invention.

### EXAMPLES

General methods. ¹H NMR spectra were obtained as solutions in an appropriate deuterated solvent such as dimethyl sulfoxide-d₆ (DMSO-d6).

LCMS Methods. Compound purity, retention time, and electrospray mass spectrometry (ESI-MS) data were determined by LC/MS analysis. LC/MS determinations were carried out using one of the following chromatographic conditions:
1) Waters BEH C₈ (1.7 μm, 2.1×50 mm) 2 to 98% acetonitrile in water (10 mM ammonium formate, pH 9), 45° C., flow rate 0.6 mL/min over 5.0 min;
2) Kinetex EVO C₁₈ (2.6 μm, 2.1×50 mm) 2 to 98% acetonitrile in water (10 mM ammonium formate, pH 9), 45° C., flow rate 0.7 mL/min over 4.0 min;
3) Kinetex EVO C₁₈ (2.6 μm 2.1×50 mm) 2 to 98% acetonitrile in water (10 mM ammonium formate, pH 9), 45° C., flow rate 1.0 mL/min over 1.5 min;
4) Waters Acquity UPLC BEH C₁₈ (1.7 μm, 30×2.1 mm) 1 to 99% acetonitrile (0.035% TFA) in water (0.05% TFA), 60° C., flow rate=1.5 mL/min over 3 min;
5) Kinetex Polar C₁₈ (2.6 μm, 3.0×50 mm) 5 to 95% acetonitrile in water (0.1% formic acid), flow rate 1.2 mL/min over 6 min;
6) SunFire C₁₈ (3.5 μm, 75×4.6 mm) initial 5 to 95% acetonitrile in water (0.1% formic acid) for 1 min then linear gradient to 95% acetonitrile for 5 min. 45° C., flow rate 1.5 mL/min over 6 min;
7) XBridge C₁₈ (5 μm, 4.6×75 mm) initial gradient 5 to 95% acetonitrile (NH₄HCO₃), 6 min run with 1 min equilibration gradient 0 to 3 min at 95% acetonitrile and hold for 3 min, flow rate 1.5 mL/min;
8) Waters CSH C₁₈ (1.7 μm, 2.1×50 mm) 2 to 98% acetonitrile in water (0.1% TFA, pH 2), 45° C., flow rate 0.6 mL/min over 5.0 min;
9) Waters CSH C₁₈ (1.7 μm, 2.1×50 mm) 2 to 95% acetonitrile in water (0.1% formic acid), 40° C., flow rate 0.8 mL/min over 4.6 min;
10) Waters BEH C₁₈ (2.5 μm, 2.1×50 mm) 2 to 95% acetonitrile in water (0.1% NH₃), 40° C., flow rate 0.8 mL/min over 4.6 min;
11) Waters BEH C₁₈ (3.5 μm, 75×4.6 mm) initial gradient 5 to 95% acetonitrile in water (0.1% formic acid) then linear gradient to 95% acetonitrile for 4 min, hold for 2 min at 95% acetonitrile, 45° C., flow rate 1.5 mL/min over 6 min;
12) Waters BEH C₁₈ (2.5 μm, 2.1×50 mm) 2 to 50% acetonitrile in water (0.1% NH₃), 40° C., flow rate 0.8 mL/min over 4.6 min;
13) Waters CSH C₁₈ (1.7 μm, 2.1×50 mm) 2 to 98% acetonitrile in water (0.1% TFA), 45° C., flow rate 1.0 mL/min over 1.5 min;
14) Waters CSH C₁₈ (1.7 μm, 2.1×50 mm) 2 to 95% acetonitrile in water (0.1% formic acid), 40° C., flow rate 0.8 mL/min over 1.4 min;
15) YMC Triart C₁₈ (3 μm, 33×2.1 mm) 2 to 98% acetonitrile in water (5 mM NH₄OAc), flow rate 1.0 mL/min over 3 min;
16) Waters BEH C₁₈ (2.5 μm, 2.1×50 mm) 2 to 95% acetonitrile in water (0.1% NH₃), 40° C., flow rate 0.8 mL/min over 1.4 min;
17) Waters Acquity UPLC BEH C₁₈ (1.7 μm, 30×2.1 mm) 1 to 99% acetonitrile (0.035% TFA) in water (0.05% TFA), 60° C., flow rate=1.5 mL/min over 5 min;
18) Waters BEH C₁₈ (2.5 μm, 2.1×50 mm) 20 to 70% acetonitrile in water (0.1% NH₃), 40° C., flow rate 0.8 mL/min over 4.60 min;
19) Kinetex Polar C₁₈ (2.6 μm, 3.0×50 mm) 5 to 95% acetonitrile in water (0.1% formic acid), flow rate 1.2 mL/min over 3 min;
20) Waters Acquity UPLC BEH C₁₈ column (1.7 μm, 30×2.1 mm) 1 to 99% acetonitrile (0.035% TFA) in water (0.05% TFA), 60° C., flow rate=1.5 mL/min over 1 min;
21) YMC Triart C₁₈ (3 μm, 33×2.1 mm) 2 to 98% acetonitrile in water (0.05% formic acid), flow rate 1.0 mL/min over 3 min;
22) Waters Acquity UPLC BEH C₁₈ (1.7 μm, 30×2.1 mm) 1 to 99% acetonitrile (0.05% ammonium formate) in water (0.05% ammonium formate), 60° C., flow rate=1.5 mL/min over 5 min;
23) Waters CSH C₁₈ (1.7 μm, 2.1×50 mm) 2 to 98% acetonitrile in water (0.1% TFA), 45° C., flow rate 0.6 mL/min over 4.0 min;
24) Acquity BEH C8 (1.7 μm, 50×2.1 mm) 2 to 98% 90:10 acetonitrile:water (0.05% formic acid), flow rate 0.8 mL/min over 3 min;
25) XBridge C₁₈ (5 μm, 50×4.6 mm) 10 to 90% acetonitrile in water (10 mM NH₄OAc), flow rate 1.2 mL/min over 6 min;
26) YMC Triart C₁₈ (3 μm, 33×2.1 mm) 5 to 95% acetonitrile in water (0.05% formic acid), flow rate 1.0 mL/min over 12 min; or
27) Waters BEH C8 (1.7 μm, 2.1×50 mm) 50 to 95% acetonitrile in water (0.1% NH₃), 40° C., flow rate 0.8 mL/min over 1.4 min.

### Abbreviations

Unless otherwise noted, or where the context dictates otherwise, the following abbreviations shall be understood to have the following meanings:

| Abbreviation | Meaning |
|---|---|
| NMR | Nuclear magnetic resonance |
| ESI-MS | Electrospray mass spectrometry |
| LC/MS | Liquid chromatography-mass spectrometry |
| UPLC | Ultra performance liquid chromatography |
| HPLC/ | High performance liquid chromatography/tandem mass |
| MS/MS | spectrometry |
| IS | Internal standard |
| HPLC | High performance liquid chromatography |
| SFC | Supercritical fluid chromatography |
| ESI | Electrospray ionization |
| kg | Kilogram |
| g | Grams |
| mg | Milligrams |
| L | Liter(s) |
| mL | Milliliters |
| μL | Microliters |
| nL | Nanoliters |
| mol | Mole |
| mmol | Millimoles |
| hr, h | Hours |
| min | Minutes |
| ms | Millisecond |
| mm | Millimeters |
| μm | Micrometers |
| nm | Nanometer |
| MHz | Megahertz |
| Hz | Hertz |
| N | Normal (concentration) |
| M | Molar (concentration) |
| mM | Millimolar (concentration) |
| μM | Micromolar (concentration) |
| ppm | Parts per million |
| % w/v | Weight-volume concentration |
| % w/w | Weight-weight concentration |
| Ac₂O | Acetic anhydride |
| BnBr | Benzyl bromide |
| t-BuOH | tert-butyl alcohol |
| CDI | 1,1′-Carbonyldiimidazole |
| DAST | (Diethylamino)sulfur trifluoride |
| DCM | Dichloromethane |
| DCE | Dichloroethane |
| DIAD | Diisopropyl azodicarboxylate |
| DIBAL | Diisobutylaluminium hydride |
| DIEA, | N,N-Diisopropyl ethyl amine |
| DIPEA | |
| DMA | N,N-Dimethylacetamide |
| DMAP | Dimethylaminopyridine |
| DMF | N,N-Dimethylformamide |
| DMSO | Dimethyl sulfoxide |
| DRG | Dorsal root ganglia |
| EtOH | Ethanol |
| EtOAc | Ethyl acetate |
| HATU | 1-[Bis(dimethylamino)methylene]-1H-1,2,3- |
| | triazolo[4,5-b]pyridinium |
| | 3-oxide hexafluorophosphate |
| EDCI | 1-Ethyl-3-(3-dimethylaminopropyl)carbodiimide |
| T3P | Propylphosphonic anhydride, i.e., 2,4,6-tripropyl- |
| | 1,3,5,2,4,6-trioxatriphosphinane 2,4,6-trioxide |
| mCPBA | Meta-Chloroperoxybenzoic acid |
| MeOH | Methanol |
| MsCl | Methanesulfonyl chloride |
| MTBE | Methyl tert-butyl ether |
| NCS | N-Chlorosuccinimide |
| NIS | N-Iodosuccinimide |
| NMP | N-Methylpyrrolidone |
| PdCl₂(dtbpf) | 1,1′-Bis(di-tert-butylphosphino)ferrocene palladium |
| | dichloride |
| PTSA | Para-toluenesulfonic acid |
| STAB | Sodium triacetoxyborohydride |
| TBAF | Tetrabutylammonium fluoride |
| TBSOTf | tert-Butyldimethylsilyl trifluoromethanesulfonate |
| TCFH | Chloro-N,N,N′,N′-tetramethylformamidinium |
| | hexafluorophosphate |
| THF | Tetrahydrofuran |
| TEA | Triethylamine |
| Tf₂O | Trifluoromethanesulfonic anhydride |
| TFA | Trifluoroacetic acid |
| TMSCl | Trimethylsilyl chloride |
| TMSCN | Trimethylsilyl cyanide |
| RB | Round bottom (flask) |
| RT | Room temperature |
| ca. | Circa (approximately) |
| E-VIPR | Electrical stimulation voltage ion probe reader |
| HEK | Human embryonic kidney |
| KIR2.1 | Inward-rectifier potassium ion channel 2.1 |
| DMEM | Dulbecco's Modified Eagle's Medium |
| FBS | Fetal bovine serum |
| NEAA | Non-essential amino acids |
| HEPES | 2-[4-(2-hydroxyethyl)piperazin-1-yl]ethanesulfonic acid |
| DiSBAC₆(3) | Bis-(1,3-dihexyl-thiobarbituric acid) trimethine oxonol |
| CC2-DMPE | Chlorocoumarin-2-dimyristoyl phosphatidylethanolamine |
| VABSC-1 | Voltage Assay Background Suppression Compound |
| HS | Human serum |
| BSA | Bovine Serum Albumin |

### Example 1—Preparation of Intermediates A-1 to A-37

### Intermediate A-1A and A-1B

### 4-benzyloxy-2-chloro-1,6-naphthyridine-5-carbonitrile and (A-1A)

### 4-benzyloxy-2-chloro-6-oxido-1,6-naphthyridin-6-ium (A-1B)

### Step 1: 4-benzyloxy-2-chloro-1,6-naphthyridine

To a mixture of 2,4-dichloro-1,6-naphthyridine (1 g, 4.88 mmol) and benzyl alcohol (5.23 g, 0.5 mL, 4.83 mmol) in DMF (10 mL) and 2-MeTHF (10 mL) at 0° C. was added portion wise sodium hydride (208 mg, 5.2 mmol) (60% in mineral oil). The reaction mixture was stirred at 0° C. for 1 h, followed by gradually warming to room temperature and stirring for 2 h. The reaction mixture was poured onto a stirring mixture of 0.1 M aqueous HCl (50 mL) and 2-MeTHF (50 mL). The layers were separated, and the aqueous layer was extracted with 2-MeTHF (2×100 mL). The combined organic layer was washed with water (2×50 mL), water/brine 1/1 (50 mL) and brine (50 mL), dried over sodium sulfate, filtered and evaporated under reduced pressure. The residue was purified by silica gel column chromatography using 0 to 35% ethyl acetate in heptanes to obtain 4-benzyloxy-2-chloro-1,6-naphthyridine (528 mg, 38%) as an off-white solid. ESI-MS m/z calc. 270.06, found 271.1 (M+1)⁺. ¹H NMR (400 MHz, CDCl₃) δ (ppm) 9.58 (s, 1H), 8.78 (d, J=5.9 Hz, 1H), 7.75 (dd, J=5.9, 0.6 Hz, 1H), 7.54-7.37 (m, 5H), 6.92 (s, 1H), 5.34 (s, 2H).

### Step 2: 4-benzyloxy-2-chloro-6-oxido-1,6-naphthyridin-6-ium (Intermediate A-1B)

To a solution of 4-benzyloxy-2-chloro-1,6-naphthyridine (1.75 g, 6.46 mmol) in DCM (14 mL) at 0° C. was added m-CPBA (1.6 g, 7.14 mmol). The resulting mixture was stirred at room temperature for 18 h. 2 M aqueous solution sodium carbonate (60 mL) and water (90 mL) was added. The aqueous layer was extracted with DCM (3×100 mL). The combined organic layer was dried over anhydrous sodium sulfate, filtered and concentrated under reduced pressure to give 4-benzyloxy-2-chloro-6-oxido-1,6-naphthyridin-6-ium (Intermediate A-1B, 1.78 g, 97%) as an off-white solid. ESI-MS m/z calc. 286.05, found 287.1 (M+1)⁺. ¹H NMR (400 MHz, CDCl₃) δ (ppm) 9.02-8.94 (m, 1H), 8.30 (dd, J=7.3, 2.1 Hz, 1H), 7.79-7.72 (m, 1H), 7.44 (d, J=2.6 Hz, 5H), 6.93 (s, 1H), 5.29 (s, 2H).

### Step 3: 4-benzyloxy-2-chloro-1,6-naphthyridine-5-carbonitrile (Intermediate A-1A)

To a solution of 4-benzyloxy-2-chloro-6-oxido-1,6-naphthyridin-6-ium (1 g, 3.49 mmol) in DCM (10 mL) under an atmosphere of nitrogen was added trimethylsilylformonitrile (1.3 mL, 9.75 mmol), followed by the addition of Et₃N (1.25 mL, 8.97 mmol). The reaction mixture was stirred at room temperature for 20 h. The reaction mixture was quenched with water and the aqueous layer was extracted with DCM (3×). The combined organic layer was dried over magnesium sulfate, filtered and concentrated. Purification via silica gel column chromatography using 0 to 30% ethyl acetate in DCM gave 4-benzyloxy-2-chloro-1,6-naphthyridine-5-carbonitrile (Intermediate A-1A, 840 mg, 81%). ESI-MS m/z calc. 295.05, found 296.1 (M+1)⁺. ¹H NMR (400 MHz, DMSO-d₆) δ (ppm) 8.91 (d, J=5.7 Hz, 1H), 8.12 (d, J=5.7 Hz, 1H), 7.67-7.58 (m, 3H), 7.50-7.34 (m, 3H), 5.61 (s, 2H).

### Intermediate A-2

### 4-benzyloxy-2-chloro-quinoline

### Step 1: 4-benzyloxy-2-chloro-quinoline (Intermediate A-2)

Sodium hydride (16.2 mg, 0.67 mmol) was added portion wise to 2-chloroquinolin-4-ol (102.3 mg, 0.57 mmol) in DMF (1 mL) at 0° C. under an atmosphere of nitrogen. The reaction mixture was stirred at for 10 min at room temperature and again cooled down at 0° C. and benzyl bromide (75 μL, 0.63 mmol) was added. The reaction mixture was stirred at room temperature for 3 h and poured onto brine (20 mL) and ethyl acetate (30 mL) was added. The layers were separated, and the organic layer was dried over sodium sulfate, filtered and concentrated under reduced pressure. Purification by silica gel column chromatography using 1 to 100% ethyl acetate in hexanes gave 4-benzyloxy-2-chloro-quinoline (139 mg, 90%) as a white solid. ESI-MS m/z calc. 269.06, found 270.11 (M+1)⁺.

### Intermediate A-3

### 8-benzyloxy-6-chloro-1-oxido-1,5-naphthyridin-1-ium (A-3)

### Step 1: 4-benzyloxy-2-chloro-1,5-naphthyridine

A solution of benzyl alcohol (140 μL, 1.353 mmol) in THF (5 mL) was cooled to 0° C. Sodium hydride (65.3 mg, 1.63 mmol) was added, and the reaction mixture was stirred for 1 hour at 0° C. 2,4-dichloro-1,5-naphthyridine (250 mg, 1.25 mmol) was added as a solution in THF (5 mL). The reaction mixture was gradually warmed to room temperature and stirred for 2 hours before diluting with ethyl acetate and saturated sodium bicarbonate solution. The organic layer was separated, dried over magnesium sulfate and concentrated. Purification by silica gel column chromatography using 0 to 30% (3:1 ethyl acetate: ethanol (w/2% NH₄OH)) in heptane gave 4-benzyloxy-2-chloro-1,5-naphthyridine (244 mg, 72%). ESI-MS m/z calc. 270.06, found 271.4 (M+1)⁺.

### Step 2: 8-benzyloxy-6-chloro-1-oxido-1,5-naphthyridin-1-ium (Intermediate A-3)

A solution of 4-benzyloxy-2-chloro-1,5-naphthyridine (400 mg, 1.48 mmol) in DCM (8 mL) was cooled to 0° C. under nitrogen atmosphere and treated with solid 3-chlorobenzenecarboperoxoic acid (433 mg, 1.93 mmol). The reaction mixture was warmed to room temperature and stirred for 22 h. The reaction was quenched with an aqueous solution of saturated sodium bicarbonate and the organic layer was separated. The aqueous layer was extracted with DCM (3×) and the combined organic layer was washed with brine (2×), dried over anhydrous sodium sulfate, filtered, and evaporated under reduced pressure. The yellow solid was dissolved in DCM (8 mL) and cooled to 0° C. under nitrogen atmosphere. m-CPBA (433 mg, 1.93 mmol) was added to the reaction and the reaction gradually warmed to room temperature and stirred for 14 h. The reaction mixture was quenched with an aqueous solution of saturated sodium bicarbonate and the organic layer was separated. The aqueous layer was extracted with DCM (3×) and the combined organic layer was washed with brine (2×), dried over anhydrous sodium sulfate, filtered, and evaporated under reduced pressure to obtain 8-benzyloxy-6-chloro-1-oxido-1,5-naphthyridin-1-ium (Intermediate A-3, 370 mg, 87%). ESI-MS m/z calc. 286.05, found 287.1 (M+1)⁺.

### Intermediate A-4

### 4-benzyloxy-2-chloro-quinoline-5-carbonitrile

### Step 1: 4-chloro-1-oxido-quinolin-1-ium-5-carbonitrile

To a flask charged with 4-chloroquinoline-5-carbonitrile (840 mg, 4.45 mmol) in DCM (12 mL), m-CPBA (1.36 g, 5.52 mmol) was added, and the reaction mixture was stirred for 2 hours at ambient temperature. The reaction mixture was quenched with saturated sodium bicarbonate solution. The aqueous layer was extracted with DCM (3×), dried over magnesium sulfate, filtered and concentrated under reduced in vacuo to give 4-chloro-1-oxido-quinolin-1-ium-5-carbonitrile (910 mg, 100%). ESI-MS m/z calc. 204.01, found 205.0 (M+1)⁺.

### Step 2: 2,4-dichloroquinoline-5-carbonitrile

A vial charged with 4-chloro-1-oxido-quinolin-1-ium-5-carbonitrile (900 mg, 4.4 mmol) and POCl₃ (4 mL, 42.91 mmol) was heated at 50° C. for 4 hours. The reaction mixture was cooled to room temperature and poured on ice. The product crashed out, which was filtered and washed with water. It was dissolved in DCM and washed with saturated sodium bicarbonate solution (2×). The organic layer was dried over magnesium sulfate, filtered and concentrated. Purified via silica gel column chromatography using 0 to 20% ethyl acetate in hexanes to obtain 2,4-dichloroquinoline-5-carbonitrile (680 mg, 69%). ESI-MS m/z calc. 221.97, found 223.0 (M+1)⁺.

### Step 3: 4-benzyloxy-2-chloro-quinoline-5-carbonitrile (Intermediate A-4)

A round bottom flask equipped with a stir bar was charged with DMF (10 mL) and cooled to 0° C. Benzyl alcohol (232 μL, 2.24 mmol) was then added followed by sodium hydride (98 mg of 60% w/w, 2.45 mmol) and the reaction was warmed to room temperature and stirred for 30 minutes. The reaction mixture was then cooled to −40° C. and 2,4-dichloroquinoline-5-carbonitrile (500 mg, 2.24 mmol) was added. The reaction was gradually warmed to room temperature and stirred overnight. The reaction mixture was cooled to 0° C. and was quenched with water, extracted with ethyl acetate (3×), washed with water, brine (3×), dried with magnesium sulfate, filtered, and concentrated. The crude material was purified by silica gel column chromatography using 0 to 30% ethyl acetate in hexanes to afford 4-benzyloxy-2-chloro-quinoline-5-carbonitrile (Intermediate A-4, 410 mg, 62%) as an off-white white solid. ESI-MS m/z calc. 294.05, found 295.6 (M+1)⁺. ¹H NMR (400 MHz, DMSO-d₆) δ (ppm) 8.23-8.15 (m, 2H), 7.92 (dd, J=8.5, 7.4 Hz, 1H), 7.67-7.57 (m, 2H), 7.48 (s, 1H), 7.46-7.32 (m, 3H), 5.56 (s, 2H). The corresponding regioisomer was also isolated 2-benzyloxy-4-chloro-quinoline-5-carbonitrile. ESI-MS m/z calc. 294.056 found 295.1 (M+1)⁺. ¹H NMR (400 MHz, DMSO-d₆) δ (ppm) 8.17 (ddd, J=14.6, 8.0, 1.3 Hz, 2H), 7.90 (dd, J=8.4, 7.4 Hz, 1H), 7.58 (s, 1H), 7.55-7.50 (m, 2H), 7.44-7.32 (m, 3H), 5.53 (s, 2H).

### Intermediate A-5

### methyl 4-benzyloxy-2-chloro-quinoline-6-carboxylate

### Step 1: methyl 4-chloro-1-oxido-quinolin-1-ium-6-carboxylate

To a flask charged with methyl 4-chloroquinoline-6-carboxylate (1 g, 4.51 mmol) in DCM (10 mL), 3-chlorobenzenecarboperoxoic acid (1.4 g, 5.68 mmol) was added and stirred for 2 hours at ambient temperature. The reaction mixture was quenched with saturated sodium bicarbonate solution and filtered. The aqueous layer was extracted with DCM (3×), dried over magnesium sulfate, filtered and concentrated under reduced vacuo to give methyl 4-chloro-1-oxido-quinolin-1-ium-6-carboxylate (1.05 g, 98%). ESI-MS m/z calc. 237.02, found 238.23 (M+1)⁺.

### Step 2: methyl 2,4-dichloroquinoline-6-carboxylate

A vial charged with methyl 4-chloro-1-oxido-quinolin-1-ium-6-carboxylate (1 g, 4.21 mmol) and POCl₃ (5 mL, 53.64 mmol) was heated at 50° C. for 4 h. The reaction mixture was cooled to room temperature and poured onto ice. The desired product crashed out, which was filtered and washed with water. It was dissolved in DCM and washed with saturated sodium bicarbonate solution (2×). The organic layer was dried over magnesium sulfate, filtered and concentrated. Purified via silica gel column chromatography using 0 to 20% ethyl acetate in hexanes to obtain methyl 2,4-dichloroquinoline-6-carboxylate (401 mg, 37%). ESI-MS m/z calc. 254.98, found 256.12 (M+1)⁺. ¹H NMR (400 MHz, DMSO-d₆) δ (ppm) 8.78 (d, J=1.9 Hz, 1H), 8.37 (dd, J=8.8, 1.9 Hz, 1H), 8.16 (d, J=8.8 Hz, 1H), 8.13 (s, 1H), 3.97 (s, 3H).

### Step 3: methyl 4-benzyloxy-2-chloro-quinoline-6-carboxylate (Intermediate A-5)

A round bottom flask equipped with a stir bar was charged with DMF (8 mL) and cooled to 0° C. Benzyl alcohol (162 μL, 1.56 mmol) was then added followed by the addition of sodium hydride (67 mg of 60% w/w, 1.67 mmol) and the reaction was warmed to room temperature and stirred for 30 minutes. The reaction mixture was then cooled to −40° C. and methyl 2,4-dichloroquinoline-6-carboxylate (400 mg, 1.56 mmol) was added. The reaction was gradually warmed to room temperature and stirred overnight. The reaction mixture was cooled to 0° C. and was quenched with water, extracted with ethyl acetate (3×). The combined organic layer was washed with brine (3×), dried with magnesium sulfate, filtered, and concentrated. The crude material was purified by reverse phase chromatography using 1 to 99% ACN in water (HCl modifier) to obtain methyl 4-benzyloxy-2-chloro-quinoline-6-carboxylate (Intermediate A-5, 110 mg, 21%). ESI-MS m/z calc. 327.06, found 328.32 (M+1)⁺. ¹H NMR (400 MHz, DMSO-d₆) δ (ppm) 8.71 (d, J=2.0 Hz, 1H), 8.26 (dd, J=8.8, 2.0 Hz, 1H), 7.99 (d, J=8.8 Hz, 1H), 7.62-7.54 (m, 2H), 7.52-7.39 (m, 3H), 7.37 (s, 1H), 5.49 (s, 2H), 3.90 (s, 3H). The corresponding regioisomer was also isolated, methyl 2-benzyloxy-4-chloro-quinoline-6-carboxylate. ESI-MS m/z calc. 327.06, found 328.42 (M+1)⁺. ¹H NMR (400 MHz, DMSO-d₆) δ (ppm) 8.70 (d, J=1.9 Hz, 1H), 8.27 (dd, J=8.7, 2.0 Hz, 1H), 7.97 (d, J=8.7 Hz, 1H), 7.54 (h, J=2.0 Hz, 3H), 7.44-7.32 (m, 3H), 5.56 (s, 2H), 3.94 (s, 3H).

### Intermediate A-6

### ethyl 4-benzyloxy-2-chloro-6-fluoro-quinoline-3-carboxylate

### Step 1: ethyl 4-chloro-6-fluoro-1-oxido-quinolin-1-ium-3-carboxylate

To a flask charged with ethyl 4-chloro-6-fluoro-quinoline-3-carboxylate (2.5 g, 9.86 mmol) in DCM (40 mL), m-CPBA (3 g, 12.17 mmol) was added, and the reaction mixture was stirred for 2 hours at ambient temperature. The reaction mixture was quenched with saturated sodium bicarbonate solution and filtered. The aqueous layer was extracted with DCM (3×), dried over magnesium sulfate, filtered and concentrated under reduced pressure to give ethyl 4-chloro-6-fluoro-1-oxido-quinolin-1-ium-3-carboxylate (2.6 g, 98%). ESI-MS m/z calc. 269.02, found 270.2 (M+1)⁺.

### Step 2: ethyl 2,4-dichloro-6-fluoro-quinoline-3-carboxylate

A vial charged with ethyl 4-chloro-6-fluoro-1-oxido-quinolin-1-ium-3-carboxylate (2.6 g, 9.64 mmol) and POCl₃ (9 mL, 96.56 mmol) was heated at 50° C. for 4 h. The reaction mixture was cooled to room temperature and poured onto ice. The desired product crashed out, which was filtered and washed with water. It was dissolved in DCM and washed with saturated sodium bicarbonate solution (2×). The organic layer was dried over magnesium sulfate, filtered and concentrated. Purified via silica gel column chromatography using 0 to 10% ethyl acetate in hexanes to obtain ethyl 2,4-dichloro-6-fluoro-quinoline-3-carboxylate (2.12 g, 76%). ESI-MS m/z calc. 286.99, found 288.14 (M+1)⁺. ¹H NMR (400 MHz, DMSO-d₆) δ (ppm) 8.20 (dd, J=9.2, 5.2 Hz, 1H), 8.02 (dd, J=9.3, 2.8 Hz, 1H), 7.97 (ddd, J=9.2, 8.3, 2.8 Hz, 1H), 4.51 (q, J=7.1 Hz, 2H), 1.38 (t, J=7.1 Hz, 3H).

### Step 3: ethyl 4-benzyloxy-2-chloro-6-fluoro-quinoline-3-carboxylate (Intermediate A-6)

A round bottom flask equipped with a stir bar was charged with DMF (40 mL) and cooled to 0° C. Benzyl alcohol (755 μL, 7.29 mmol) was added followed by the addition of sodium hydride (320 mg of 60% w/w, 8 mmol). The reaction was warmed to room temperature and stirred for 30 minutes. The reaction mixture was then cooled to −40° C. and ethyl 2,4-dichloro-6-fluoro-quinoline-3-carboxylate (2.1 g, 7.29 mmol) was added. The reaction was gradually warmed to room temperature and stirred overnight. The reaction mixture was cooled to 0° C. then was quenched with water and extracted with ethyl acetate (3×), washed with brine (3×), dried over magnesium sulfate, filtered, and concentrated. The crude material was purified by silica gel column chromatography using 0 to 10% ethyl acetate in hexanes to afford ethyl 4-benzyloxy-2-chloro-6-fluoro-quinoline-3-carboxylate (Intermediate A-6, 1.69 g, 64%). ESI-MS m/z calc. 359.07, found 361.3 (M+2)⁺. ¹H NMR (400 MHz, DMSO-d₆) δ (ppm) 8.08 (dd, J=9.2, 5.2 Hz, 1H), 7.88-7.75 (m, 2H), 7.55-7.49 (m, 2H), 7.49-7.38 (m, 3H), 5.35 (s, 2H), 4.42 (q, J=7.1 Hz, 2H), 1.32 (t, J=7.1 Hz, 3H).

### Intermediate A-7

### 4-benzyloxy-2-chloro-6-fluoro-5-methoxy-quinoline

### Step 1: 8-bromo-6-fluoro-4-hydroxy-5-methoxy-1H-quinolin-2-one

A vial charged with 2-bromo-4-fluoro-5-methoxy-aniline (3 g, 13.63 mmol) and 2,2-dimethyl-1,3-dioxane-4,6-dione (2 g, 13.88 mmol) was heated (neat) at 80° C. for 16 hours to obtain 3-(2-bromo-4-fluoro-5-methoxy-anilino)-3-oxo-propanoic acid. ESI-MS m/z calc. 304.97, found 308.1 (M+3)⁺. The reaction mixture was subjected to vacuum by rotary evaporation to remove any acetone formed. Eatons Reagent (15 mL, 94.52 mmol) was added, and the mixture was stirred at 80° C. for 18 hours. The reaction mixture was poured onto ice cold water and was stirred for 5 minutes and was diluted with water and solids were filtered. The solid was dissolved with 0.5 N sodium hydroxide and washed with toluene (2×). The pH was adjusted to 3 with concentrated HCl to give solids which were filtered and washed with water to give 8-bromo-6-fluoro-4-hydroxy-5-methoxy-1H-quinolin-2-one (2 g, 51%). ESI-MS m/z calc. 286.96, found 290.1 (M+3)⁺. ¹H NMR (400 MHz, DMSO-d₆) δ (ppm) 11.27 (s, 1H), 9.65 (s, 1H), 7.99 (d, J=10.4 Hz, 1H), 5.84 (s, 1H), 3.86 (s, 3H).

### Step 2: 6-fluoro-4-hydroxy-5-methoxy-1H-quinolin-2-one

A flask charged with 8-bromo-6-fluoro-4-hydroxy-5-methoxy-1H-quinolin-2-one (750 mg, 2.6 mmol) and 10% Pd/C (250 mg, 2.35 mmol) was evacuated under vacuum and backfilled with nitrogen. To it was added ethanol (10 mL) and Et₃N (725 μL, 5.2 mmol) and the reaction mixture was evacuated under nitrogen (twice) and then hydrogen (twice). The reaction mixture was stirred under an atmosphere of hydrogen by placing a balloon for 5 hours. The reaction mixture was filtered through a plug of celite, and the solvent evaporated to obtain 6-fluoro-4-hydroxy-5-methoxy-1H-quinolin-2-one (540 mg, 99%) ESI-MS m/z calc. 209.05, found 210.1 (M+1)⁺. ¹H NMR (400 MHz, DMSO-d₆) δ (ppm) 11.26 (s, 1H), 7.45 (dd, J=10.8, 9.1 Hz, 1H), 7.02 (dd, J=9.1, 4.3 Hz, 1H), 5.73 (s, 1H), 3.85 (d, J=0.9 Hz, 3H).

### Step 3: 2,4-dichloro-6-fluoro-5-methoxy-quinoline

A flask charged with 6-fluoro-4-hydroxy-5-methoxy-1H-quinolin-2-one (540 mg, 2.58 mmol) and POCl₃ (6 mL, 64.37 mmol) was heated at 110° C. for 3 hours. The reaction mixture was cooled to room temperature and poured onto ice and stirred for 5 minutes. The product crashed out, which was filtered and the solid was dissolved in DCM. The organic layer was washed with saturated sodium bicarbonate, dried over magnesium sulfate, filtered and concentrated. Purification via silica gel column chromatography using 0 to 10% ethyl acetate in hexanes gave 2,4-dichloro-6-fluoro-5-methoxy-quinoline (370 mg, 58%). ESI-MS m/z calc. 244.98, found 246.1 (M+1)⁺. ¹H NMR (400 MHz, DMSO-d₆) δ (ppm) 7.98-7.91 (m, 1H), 7.90 (s, 1H), 7.89-7.82 (m, 1H), 3.96 (d, J=1.1 Hz, 3H).

### Step 4: 4-benzyloxy-2-chloro-6-fluoro-5-methoxy-quinoline (Intermediate A-7)

Benzyl alcohol (48 μL, 0.46 mmol) was dissolved in THF (1 mL) and cooled to 0° C. in an ice bath. Sodium hydride (22 mg of 60% w/w, 0.55 mmol) was added and the reaction was warmed to room temperature over one hour. The reaction was cooled again in an ice bath and a solution of 2,4-dichloro-6-fluoro-5-methoxy-quinoline (103 mg, 0.4186 mmol) in THF (0.6 mL) was added dropwise. The reaction was warmed to room temperature and stirred for 16 hours. The reaction mixture was quenched with water and the aqueous layer was extracted with ethyl acetate. The combined organic layer was washed with brine, dried over sodium sulfate, filtered and concentrated. The crude material was purified by silica gel column chromatography eluting with 0 to 10% ethyl acetate in hexanes to give 4-benzyloxy-2-chloro-6-fluoro-5-methoxy-quinoline (Intermediate A-7, 92 mg, 69%) as a white solid. ESI-MS m/z calc. 317.06, found 318.2 (M+1)⁺. ¹H NMR (400 MHz, DMSO-d₆) δ (ppm) 7.82-7.75 (m, 1H), 7.73-7.67 (m, 1H), 7.62-7.57 (m, 2H), 7.50-7.44 (m, 2H), 7.43-7.37 (m, 1H), 7.25 (s, 1H), 5.41 (s, 2H), 3.75 (s, 3H).

### Intermediate A-8

### 4-benzyloxy-2-chloro-7-fluoro-5-methoxy-quinoline

### Step 1: 5-fluoro-4-hydroxy-7-methoxy-1H-quinolin-2-one and 7-fluoro-4-hydroxy-5-methoxy-1H-quinolin-2-one

A vial charged with 3-fluoro-5-methoxy-aniline (4 g, 28.34 mmol) and 2,2-dimethyl-1,3-dioxane-4,6-dione (4.1 g, 28.45 mmol) was heated at 80° C. for 16 hours to obtain 3-(3-fluoro-5-methoxy-anilino)-3-oxo-propanoic acid. ESI-MS m/z calc. 227.06, found 228.16 (M+1)⁺. Eatons Reagent (20 mL, 126.0 mmol) was added, and the mixture was stirred at 70° C. for 18 hours. The reaction mixture was poured onto ice cold water and stirred for 10 minutes. It was diluted with water and solids were filtered. The solid was dissolved with 0.5 N sodium hydroxide and washed with toluene (2×). The pH was adjusted to 1 with concentrated HCl to give solids which were filtered and washed with water and dried over high vacuum to give the mixture of 5-fluoro-4-hydroxy-7-methoxy-1H-quinolin-2-one and 7-fluoro-4-hydroxy-5-methoxy-1H-quinolin-2-one (2 g, 34%). ESI-MS m/z calc. 209.05, found 210.1 (M+1)⁺. which was carried to the next step without further purification.

### Step 2: 2,4-dichloro-7-fluoro-5-methoxy-quinoline and 2,4-dichloro-5-fluoro-7-methoxy-quinoline

To a flask charged with a mixture of 5-fluoro-4-hydroxy-7-methoxy-1H-quinolin-2-one and 7-fluoro-4-hydroxy-5-methoxy-1H-quinolin-2-one (2 g, 9.56 mmol) was added POCl₃ (9 mL, 96.56 mmol) and the reaction mixture was heated at 100° C. for 2 hours. The reaction mixture was cooled to room temperature and poured onto ice. The desired product crashed out which was filtered and dissolved in DCM. The organic layer was washed with saturated sodium bicarbonate solution and the dried over magnesium sulfate, filtered and concentrated. The crude material was purified via silica gel column chromatography using 0 to 8% ethyl acetate in hexanes to obtain 2,4-dichloro-7-fluoro-5-methoxy-quinoline (490 mg, 21%). ESI-MS m/z calc. 244.98, found 246.1 (M+1)⁺. ¹H NMR (400 MHz, DMSO-d₆) δ (ppm) 7.71 (s, 1H), 7.33 (dd, J=9.6, 2.5 Hz, 1H), 7.20 (dd, J=11.4, 2.5 Hz, 1H), 3.98 (s, 3H); and 2,4-dichloro-5-fluoro-7-methoxy-quinoline (596 mg, 25%). ESI-MS m/z calc. 244.98105, found 246.1 (M+1)⁺. ¹H NMR (400 MHz, DMSO-d₆) δ (ppm) 7.74 (s, 1H), 7.34-7.27 (m, 2H), 3.95 (s, 3H). The product structures were confirmed by NOESY NMR analysis.

### Step 3: 4-benzyloxy-2-chloro-7-fluoro-5-methoxy-quinoline (Intermediate A-8)

A round bottom flask equipped with a stir bar was charged with benzyl alcohol (225 μL, 2.17 mmol), THF (5 mL) and DMF (250 μL). It was cooled to 0° C. and sodium hydride (87 mg of 60% w/w, 2.17 mmol) was added and the reaction was warmed to room temperature and stirred for 30 minutes. The reaction mixture was again cooled to 0° C. and 2,4-dichloro-7-fluoro-5-methoxy-quinoline (485 mg, 1.97 mmol) was added dropwise as solution in THF (3 mL). The reaction mixture was gradually warmed to room temperature and stirred for 16 hours. The reaction mixture was quenched with water and the aqueous layer was extracted with ethyl acetate (3×). The combined organic layer was washed with brine, dried over magnesium sulfate, filtered and concentrated. Purification via silica gel column chromatography using 0 to 10% ethyl acetate in hexanes gave 4-benzyloxy-2-chloro-7-fluoro-5-methoxy-quinoline (Intermediate A-8, 318 mg, 51%). ESI-MS m/z calc. 317.06, found 318.3 (M+1)⁺. ¹H NMR (400 MHz, DMSO-d₆) δ (ppm) 7.61-7.53 (m, 2H), 7.46 (dd, J=8.4, 6.8 Hz, 2H), 7.41-7.33 (m, 1H), 7.17 (dd, J=9.9, 2.5 Hz, 1H), 7.10 (s, 1H), 7.02 (dd, J=11.6, 2.5 Hz, 1H), 5.38 (s, 2H), 3.94 (s, 3H). ¹⁹F NMR (376 MHz, DMSO-d₆) δ (ppm) −106.77 (dd, J=11.6, 9.8 Hz). The corresponding regioisomer was also isolated, 2-benzyloxy-4-chloro-7-fluoro-5-methoxy-quinoline (75 mg, 12%). ESI-MS m/z calc. 317.06, found 318.3 (M+1)⁺. ¹H NMR (400 MHz, DMSO-d₆) δ (ppm) 7.57-7.48 (m, 2H), 7.45-7.31 (m, 3H), 7.17-7.11 (m, 2H), 6.99 (dd, J=11.4, 2.5 Hz, 1H), 5.47 (s, 2H), 3.94 (s, 3H). ¹⁹F NMR (376 MHz, DMSO-d₆) δ (ppm) −106.85 (t, J=10.6 Hz).

### Intermediate A-9

### 4-benzyloxy-2-chloro-5-fluoro-7-methoxy-quinoline

### Step 1: 4-benzyloxy-2-chloro-5-fluoro-7-methoxy-quinoline (Intermediate A-9)

A round bottom flask equipped with a stir bar was charged with benzyl alcohol (273 μL, 2.64 mmol), THF (6 mL) and DMF (300 μL) was cooled to 0° C. and sodium hydride (106 mg of 60% w/w, 2.65 mmol) was added and the reaction was warmed to room temperature and stirred for 30 minutes. The reaction was again cooled to 0° C. and 2,4-dichloro-5-fluoro-7-methoxy-quinoline (590 mg, 2.39 mmol) (Intermediate A-8, Step 2) was added dropwise as solution in THF (2.5 mL). The reaction mixture was warmed to room temperature and stirred for 48 hours. The reaction mixture was quenched with water and the aqueous layer was extracted with ethyl acetate (3×). The combined organic layer was washed with brine, dried over magnesium sulfate, filtered and concentrated. Purification via silica gel column chromatography using 0 to 10% ethyl acetate in hexanes gave 4-benzyloxy-2-chloro-5-fluoro-7-methoxy-quinoline (Intermediate A-9, 277 mg, 36%). ESI-MS m/z calc. 317.06, found 318.3 (M+1)⁺. ¹H NMR (400 MHz, DMSO-d₆) δ (ppm) 7.56-7.50 (m, 2H), 7.48-7.41 (m, 2H), 7.41-7.34 (m, 1H), 7.17 (dd, J=2.6, 0.9 Hz, 1H), 7.12 (s, 1H), 7.06 (dd, J=13.5, 2.5 Hz, 1H), 5.40 (s, 2H), 3.91 (s, 3H). The corresponding regioisomer was also isolated 2-benzyloxy-4-chloro-5-fluoro-7-methoxy-quinoline (150 mg, 20%). ESI-MS m/z calc. 317.06, found 318.3 (M+1)⁺.

### Intermediate A-10

### 4-benzyloxy-2-chloro-7-fluoro-quinoline-5-carbonitrile

### Step 1: 5-bromo-7-fluoro-quinoline-2,4-diol and 7-bromo-5-fluoro-quinoline-2,4-diol

3-bromo-5-fluoro-aniline (9.72 g, 51.15 mmol) and 2,2-dimethyl-1,3-dioxane-4,6-dione (7.39 g, 51.27 mmol) were combined and heated at 80° C. for 16 h to give 3-(3-bromo-5-fluoro-anilino)-3-oxo-propanoic acid. To this mixture was added Eaton's Reagent (50 mL, 315 mmol) and the reaction mixture was heated at 80° C. for an additional 16 h. The reaction was poured over ice and stirred for 20 min. The resulting brown solid was collected via filtration. The solids were stirred in 0.5 M NaOH (600 mL) while heating to 50° C. to allow majority of solids to dissolve. The dark brown solids were filtered and discarded. The resulting yellow solution was cooled and acidified with concentrated HCl to pH 1. The resulting fine precipitate was collected via filtration. The wet solid was suspended in acetonitrile and evaporated to dryness to give a 2:1 mixture of 5-bromo-7-fluoro-quinoline-2,4-diol and 7-bromo-5-fluoro-quinoline-2,4-diol (6.03 g, 46%) as a brown solid. ESI-MS m/z calc. 256.95, found 260.0 (M+3)⁺.

### Step 2: 5-bromo-2,4-dichloro-7-fluoro-quinoline and 7-bromo-2,4-dichloro-5-fluoro-quinoline

A mixture of 5-bromo-7-fluoro-quinoline-2,4-diol and 7-bromo-5-fluoro-quinoline-2,4-diol (3.26 g, 12.63 mmol) was dissolved in POCl₃ (20 mL, 214.6 mmol) and heated at 100° C. for 16 h. The reaction was poured over ice and stirred for 30 minutes. The resulting solid was collected via filtration and washed with water. The solid was further purified by silica gel chromatography eluting with 0-10% ethyl acetate in hexanes to give a 5:1 mixture of 5-bromo-2,4-dichloro-7-fluoro-quinoline and 7-bromo-2,4-dichloro-5-fluoro-quinoline (2.26 g, 61%). ESI-MS m/z calc. 292.88, found 294.0 (M+1)⁺. Major: ¹H NMR (400 MHz, DMSO-d₆) δ (ppm) 8.19 (dd, J=8.5, 2.7 Hz, 1H), 8.00 (s, 1H), 7.93 (dd, J=9.1, 2.8 Hz, 1H). Minor ¹H NMR (400 MHz, DMSO-d₆) δ (ppm) 8.15 (t, J=1.6 Hz, 1H), 8.02 (s, 1H), 7.90 (dd, J=1.9 Hz, 1H).

### Step 3: 2,4-dichloro-7-fluoro-quinoline-5-carbonitrile

In a 20-mL microwave vial, 5-bromo-2,4-dichloro-7-fluoro-quinoline and 7-bromo-2,4-dichloro-5-fluoro-quinoline (892.8 mg, 3.03 mmol) and CuCN (310.5 mg, 3.47 mmol) were mixed with NMP (10 mL). The resulting mixture was degassed with nitrogen gas sparging for 5 min. It was then stirred at 110° C. for 23 h. It was cooled to room temperature and quenched with water (50 mL). The aqueous layer was extracted with ethyl acetate (3×50 mL). The combined organic layer was washed with water (50 mL) and brine (50 mL), dried over sodium sulfate, filtered, and evaporated in vacuo. Purification by silica gel chromatography (220 g of silica) using a gradient eluent of 0 to 10% ethyl acetate in hexanes gave two products: 2,4-dichloro-7-fluoro-quinoline-5-carbonitrile (310 mg, 42%). ESI-MS m/z calc. 239.96, found 241.0 (M+1)⁺. ¹H NMR (400 MHz, DMSO-d₆) δ (ppm) 8.55 (dd, J=8.4, 2.7 Hz, 1H), 8.29 (dd, J=9.1, 2.7 Hz, 1H), 8.16 (s, 1H) ¹⁹F NMR (376 MHz, DMSO-d₆) δ (ppm) −106.98 (t, J(F−H)=8.9 Hz, 1F); and 2,4-dichloro-5-fluoro-quinoline-7-carbonitrile (10.3 mg, 1%). ESI-MS m/z calc. 239.96573, found 241.0 (M+1)⁺. ¹H NMR (400 MHz, DMSO-d₆) δ (ppm) 8.54 (d, J=1.4 Hz, 1H), 8.19 (s, 1H), 8.10 (dd, J=12.1, 1.5 Hz, 1H)¹⁹F NMR (376 MHz, DMSO-d₆) δ (ppm) −108.91 (d, J(F−H)=12.2 Hz, 1F),

### Step 4: 4-benzyloxy-2-chloro-7-fluoro-quinoline-5-carbonitrile (Intermediate A-10)

In a 20-mL vial, 2,4-dichloro-7-fluoro-quinoline-5-carbonitrile (309 mg, 1.28 mmol) was dissolved in DMF (6.0 mL), to which benzyl alcohol (150 μL, 1.45 mmol) was added. The resulting solution was cooled to 0° C., after which sodium hydride (60 mg of 60% w/w, 1.5 mmol) was added in one portion. The resulting mixture was stirred at 0° C. for 2 min, and then warmed to room temperature over 15 h by removing the ice-water bath. It was then quenched with water (30 mL) and extracted with 1:1 Ethyl acetate: hexanes (3×30 mL). The combined organic layer was washed with water (2×30 mL) and brine (30 mL), then dried over sodium sulfate, filtered, and evaporated in vacuo. Purification by silica gel chromatography (24 g of silica) using a gradient eluent of 0 to 30% ethyl acetate in hexanes gave an off-white solid: 4-benzyloxy-2-chloro-7-fluoro-quinoline-5-carbonitrile (Intermediate A-10, 184.8 mg, 32%). ESI-MS m/z calc. 312.05, found 313.2 (M+1)⁺. ¹H NMR (400 MHz, DMSO-d₆) δ (ppm) 8.29 (dd, J=8.4, 2.6 Hz, 1H), 8.08 (dd, J=9.4, 2.7 Hz, 1H), 7.61 (d, J=7.4 Hz, 2H), 7.53-7.30 (m, 4H), 5.56 (s, 2H) ¹⁹F NMR (376 MHz, DMSO-d₆) δ (ppm) −108.08 (t, J(F−H)=9.0 Hz, 1F).

### Intermediate A-11

### 4-benzyloxy-2-chloro-6,7-difluoro-5-methoxy-quinoline

### Step 1: 5,6-difluoro-4-hydroxy-7-methoxy-1H-quinolin-2-one and 6,7-difluoro-4-hydroxy-5-methoxy-1H-quinolin-2-one

A vial charged with 3,4-difluoro-5-methoxy-aniline (2.2 g, 13.82 mmol) and 2,2-dimethyl-1,3-dioxane-4,6-dione (2 g, 13.88 mmol) was heated at 80° C. for 16 hours to obtain 3-(3,4-difluoro-5-methoxy-anilino)-3-oxo-propanoic acid. ESI-MS m/z calc. 245.05, found 246.1 (M+1)⁺. Eatons Reagent (10 mL, 63.01 mmol) was added, and the mixture was stirred at 70° C. for 18 hours. The reaction mixture was poured onto ice cold water and stirred for 10 minutes. It was diluted with water and solids were filtered. The solid was dissolved with 0.5 N sodium hydroxide and washed with toluene (2×). The pH was adjusted to pH 1 with concentrated HCl to give solids which were filtered and washed with water. The residue was slurried in ACN and the solvent was evaporated to give 1:4 mixture of 5,6-difluoro-4-hydroxy-7-methoxy-1H-quinolin-2-one ESI-MS m/z calc. 227.0394, found 228.1 and 6,7-difluoro-4-hydroxy-5-methoxy-1H-quinolin-2-one (1.9 g, 61%). ESI-MS m/z calc. 227.04, found 228.1 (M+1)⁺.

### Step 2: 2,4-dichloro-6,7-difluoro-5-methoxy-quinoline and 2,4-dichloro-5,6-difluoro-7-methoxy-quinoline

To a flask charged with a mixture of 5,6-difluoro-4-hydroxy-7-methoxy-1H-quinolin-2-one and 6,7-difluoro-4-hydroxy-5-methoxy-1H-quinolin-2-one (1.9 g, 8.36 mmol) was added POCl₃ (10 mL, 107.3 mmol) and the reaction mixture was heated at 100° C. for 2 h. The reaction mixture was cooled to room temperature and poured onto ice. The desired product crashed out and was filtered and dissolved in DCM. The organic layer was washed with saturated sodium bicarbonate solution, dried over magnesium sulfate, filtered and concentrated. The crude material was purified via silica gel column chromatography using 0 to 8% ethyl acetate in hexanes (220 g column) to obtain 2,4-dichloro-6,7-difluoro-5-methoxy-quinoline (110 mg, 5%). ESI-MS m/z calc. 262.97, found 264.0 (M+1)⁺. ¹H NMR (400 MHz, DMSO-d₆) δ (ppm) 7.95-7.89 (m, 2H), 4.03 (d, J=1.2 Hz, 3H) and 2,4-dichloro-5,6-difluoro-7-methoxy-quinoline (1.06 g, 48%). ESI-MS m/z calc. 262.97, found 264.0 (M+1)⁺. ¹H NMR (400 MHz, DMSO-d₆) δ (ppm) 7.82 (s, 1H), 7.52 (dd, J=7.7, 2.0 Hz, 1H), 4.05 (s, 3H). ¹⁹F NMR (376 MHz, DMSO-d₆) δ (ppm) −140.48 (dd, J=17.3, 2.5 Hz), −155.14 (dd, J=17.3, 7.7 Hz). The product structures were confirmed by NOESY NMR analysis.

### Step 3: 4-benzyloxy-2-chloro-6,7-difluoro-5-methoxy-quinoline (Intermediate A-11)

To a round bottom flask equipped with a stir bar and charged with benzyl alcohol (43 μL, 0.42 mmol), THF (1 mL) and DMF (50 μL) was added sodium hydride (17 mg of 60% w/w, 0.43 mmol) at 0° C. The reaction mixture was warmed to room temperature and allowed to stir for 30 minutes. The reaction was again cooled to 0° C. and 2,4-dichloro-6,7-difluoro-5-methoxy-quinoline (100 mg, 0.38 mmol) was added dropwise as solution in THF (1 mL). The reaction mixture was gradually warmed to room temperature and stirred for 16 hours. The reaction mixture was quenched with water and the aqueous layer was extracted with ethyl acetate (3×). The combined organic layer was washed with brine, dried over magnesium sulfate, filtered and concentrated. Purification via silica gel column chromatography using 0 to 10% ethyl acetate in hexanes gave 4-benzyloxy-2-chloro-6,7-difluoro-5-methoxy-quinoline (Intermediate A-11, 44 mg, 35%). ESI-MS m/z calc. 335.05, found 336.3 (M+1)⁺. ¹H NMR (400 MHz, DMSO-d₆) δ (ppm) 7.73 (dd, J=11.3, 7.4 Hz, 1H), 7.61-7.55 (m, 2H), 7.50-7.44 (m, 2H), 7.44-7.37 (m, 1H), 7.27 (s, 1H), 5.41 (s, 2H), 3.79 (s, 3H). ¹⁹F NMR (376 MHz, DMSO-d₆) δ (ppm) −130.72 (dd, J=22.5, 11.4 Hz), −155.02 (dd, J=22.5, 7.4 Hz).

### Intermediate A-12

### 4-benzyloxy-2-chloro-5,6-difluoro-7-methoxy-quinoline

### Step 1: 4-benzyloxy-2-chloro-5,6-difluoro-7-methoxy-quinoline (Intermediate A-12)

To a round bottom flask equipped with a stir bar and charged with benzyl alcohol (432 μL, 4.17 mmol), THF (10 mL) and DMF (500 μL) was added sodium hydride (167 mg of 60% w/w, 4.17 mmol) at 0° C. The reaction mixture was warmed to room temperature and allowed to stir for 30 minutes. The reaction was again cooled to 0° C. and 2,4-dichloro-5,6-difluoro-7-methoxy-quinoline (1 g, 3.79 mmol) (Intermediate A-11, Step 2), was added dropwise as solution in THF (6 mL). The reaction mixture was warmed to room temperature and stirred for 16 hours. The reaction mixture was quenched with water and the aqueous layer was extracted with ethyl acetate (3×). The combined organic layer was washed with brine, dried over magnesium sulfate, filtered and concentrated. Purification via silica gel column chromatography using 0 to 10% ethyl acetate in hexanes gave 4-benzyloxy-2-chloro-5,6-difluoro-7-methoxy-quinoline (Intermediate A-12, 496 mg, 39%). ESI-MS m/z calc. 335.05, found 336.2 (M+1)⁺. ¹H NMR (400 MHz, DMSO-d₆) δ (ppm) 7.57-7.50 (m, 2H), 7.45 (dd, J=8.2, 6.6 Hz, 2H), 7.42-7.35 (m, 2H), 7.21 (s, 1H), 5.42 (s, 2H), 4.01 (s, 3H).

### Intermediate A-13

### 4-benzyloxy-2-chloro-5-fluoro-6-methoxy-quinoline

### Step 1: 8-bromo-5-fluoro-4-hydroxy-6-methoxy-1H-quinolin-2-one

A vial charged with 2-bromo-5-fluoro-4-methoxy-aniline (3.2 g, 14.54 mmol) and 2,2-dimethyl-1,3-dioxane-4,6-dione (2.5 g, 17.35 mmol) was heated (neat) at 80° C. for 16 hours to obtain 3-(2-bromo-5-fluoro-4-methoxy-anilino)-3-oxo-propanoic acid. ESI-MS m/z calc. 304.97, found 308.0 (M+3)⁺. The reaction mixture was subjected to vacuum by rotary evaporation to remove any acetone formed. Eatons Reagent (16 mL, 100.8 mmol) was added, and the mixture was stirred at 80° C. for 18 hours. The reaction mixture was poured onto ice cold water and stirred for 10 minutes. It was diluted with water and solids were filtered. The solid was dissolved with 0.5 N sodium hydroxide and washed with toluene (2×). The pH was adjusted to 3 with concentrated HCl to give solids which were filtered and slurried in ACN. The solvent was evaporated to obtain 8-bromo-5-fluoro-4-hydroxy-6-methoxy-1H-quinolin-2-one (1.69 g, 40%). ESI-MS m/z calc. 286.96, found 290.0 (M+3)⁺.

### Step 2: 8-bromo-2,4-dichloro-5-fluoro-6-methoxy-quinoline

A flask charged with 8-bromo-5-fluoro-4-hydroxy-6-methoxy-1H-quinolin-2-one (1.7 g, 5.92 mmol) and POCl₃ (5 mL, 53.64 mmol) was heated at 100° C. for 16 hours. The reaction mixture was cooled to room temperature and concentrated under reduced pressure. Ice chips were added to the crude. The aqueous layer was extracted with DCM. The combined organic layer was dried over sodium sulfate, filtered, concentrated and purified by silica gel column chromatography using 0 to 75% ethyl acetate in hexane to provide 8-bromo-2,4-dichloro-5-fluoro-6-methoxy-quinoline (627 mg, 32%). ESI-MS m/z calc. 322.89, found 325.90 (M+3)⁺.

### Step 3: 2,4-dichloro-5-fluoro-6-methoxy-quinoline

A flask charged with 8-bromo-2,4-dichloro-5-fluoro-6-methoxy-quinoline (672 mg, 2.07 mmol) and Pd/C (200 mg of 10% w/w, 0.19 mmol) was evacuated under vacuum and backfilled with nitrogen. To it was added ethanol (10 mL) and Et₃N (600 μL, 4.31 mmol) and the reaction mixture was evacuated under nitrogen (twice) and then Hydrogen (twice). The reaction mixture was stirred under an atmosphere of hydrogen by placing a balloon for 5 hours. The reaction mixture was filtered and concentrated. The crude material was purified by silica gel column chromatography using 1 to 30% ethyl acetate in hexane to obtain 2,4-dichloro-5-fluoro-6-methoxy-quinoline (179.5 mg, 34%). ESI-MS m/z calc. 244.98, found 246.0 (M+1)⁺. ¹H NMR (400 MHz, DMSO-d₆) δ (ppm) 7.97-7.92 (m, 1H), 7.91-7.87 (m, 1H), 7.84 (s, 1H), 4.03 (s, 3H).

### Step 4: 4-benzyloxy-2-chloro-5-fluoro-6-methoxy-quinoline (Intermediate A-13)

To a stirred solution of benzyl alcohol (80 μL, 0.77 mmol) in DMF (500 μL) at 0° C. under a nitrogen stream was added sodium hydride (46 mg, 1.15 mmol). The mixture was stirred at 0° C. for 1 h and it was then added slowly to a solution of 2,4-dichloro-5-fluoro-6-methoxy-quinoline (190 mg, 0.74 mmol) in DMF (5 mL). The resulting mixture was stirred at room temperature for 16 h and quenched with water. The mixture was filtered and purified by reverse phase preparative chromatography (C₁₈) using a gradient eluent of 30 to 70% acetonitrile in water containing 5 mM hydrochloric acid to give 4-benzyloxy-2-chloro-5-fluoro-6-methoxy-quinoline (Intermediate A-13, 70.9 mg, 29%). ESI-MS m/z calc. 317.06, found 318.1 (M+1)⁺. ¹H NMR (400 MHz, DMSO-d₆) δ (ppm) 7.78-7.70 (m, 1H), 7.69-7.63 (m, 1H), 7.47 (d, J=7.2 Hz, 2H), 7.41-7.34 (m, 2H), 7.31 (t, J=7.3 Hz, 1H), 7.12 (s, 1H), 5.36 (s, 2H), 3.89 (s, 3H).

### Intermediate A-14

### 4-benzyloxy-2-bromo-5-chloro-quinoline

### Step 1: 4,5-dichloro-1-oxido-quinolin-1-ium

To a flask charged with 4,5-dichloroquinoline (500 mg, 2.52 mmol) in DCM (5 mL), m-CPBA (750 mg, 3.04 mmol) was added and stirred for 2 hours at ambient temperature. The reaction mixture was quenched with sat sodium bicarbonate solution. The aqueous layer was extracted with DCM (3×). The combined organic layer was dried over magnesium sulfate, filtered and concentrated under reduced in vacuo to give 4,5-dichloro-1-oxido-quinolin-1-ium (530 mg, 98%). ESI-MS m/z calc. 212.97, found 214.07 (M+1)⁺.

### Step 2: 2-bromo-4,5-dichloro-quinoline

To a solution of 4,5-dichloro-1-oxido-quinolin-1-ium (260 mg, 1.21 mmol) in DCM (3.5 mL) was added phosphoryl tribromide (135 mg, 0.47 mmol) at room temperature and the reaction mixture was stirred for 16 h. The reaction mixture was poured onto ice cold water and neutralized with potassium carbonate. The aqueous layer was extracted with DCM (2×), dried over magnesium sulfate, filtered and concentrated. The crude product was purified 0 to 10% ethyl acetate in hexanes to obtain 2-bromo-4,5-dichloro-quinoline (160 mg, 48%). ESI-MS m/z calc. 274.89, found 278.0 (M+3)⁺.

### Step 3: 4-benzyloxy-2-bromo-5-chloro-quinoline (Intermediate A-14)

A round bottom flask equipped with a stir bar was charged with DMF (3 mL) and cooled to 0° C. Benzyl alcohol (60 μL, 0.58 mmol) was added followed by the addition of sodium hydride (26 mg of 60% w/w, 0.65 mmol). The reaction was warmed to room temperature and stirred for 30 minutes. The reaction was cooled to 0° C. and 2-bromo-4,5-dichloro-quinoline (160 mg, 0.57 mmol) was added. The reaction mixture was gradually warmed to room temperature and stirred overnight. The reaction mixture was quenched with water and the aqueous layer was extracted with ethyl acetate (3×). The combined organic layer was washed with brine, dried over magnesium sulfate, filtered and concentrated. Purified via silica gel column chromatography using 0 to 10% ethyl acetate in hexanes to obtain 4-benzyloxy-2-bromo-5-chloro-quinoline (Intermediate A-14, 32 mg, 16%) ESI-MS m/z calc. 346.97, found 350.1 (M+3)⁺.

### Intermediate A-15

### 4-benzyloxy-2-chloro-quinoline-7-carbonitrile

### Step 1: 2,4-dichloroquinoline-7-carbonitrile

A solution of 4-chloroquinoline-7-carbonitrile (1000 mg, 5.30 mmol) in DCM (20 mL) was treated with 3-chlorobenzenecarboperoxoic acid (1.1 g, 6.37 mmol) and stirred at room temperature overnight. The obtained mixture was quenched with saturated sodium bicarbonate solution and extracted with DCM. The organic layer was dried and evaporated in vacuo to afford 4-chloro-1-oxido-quinolin-1-ium-7-carbonitrile (1.01 g, 93%). ¹H NMR (400 MHz, CDCl₃) δ (ppm) 9.12 (d, J=1.6 Hz, 1H), 8.48 (d, J=6.6 Hz, 1H), 8.34 (d, J=8.7 Hz, 1H), 7.91 (dd, J=8.7, 1.6 Hz, 1H), 7.52 (d, J=6.6 Hz, 1H). The obtained intermediate was treated with POCl₃ (7 mL, 75.10 mmol) and heated at 50° C. for 4 h. The reaction mixture was cooled to room temperature and quenched with ice. The aqueous layer was extracted with DCM and washed with saturated sodium bicarbonate solution. The organic layer was dried over sodium sulfate, filtered and concentrated to give 2,4-dichloroquinoline-7-carbonitrile (914 mg, 77%). ESI-MS m/z calc. 221.98, found 223.0 (M+1)⁺. ¹H NMR (400 MHz, CDCl₃) δ (ppm) 8.39 (d, J=1.6 Hz, 1H), 8.33 (d, J=8.6 Hz, 1H), 7.82 (dd, J=8.7, 1.6 Hz, 1H), 7.66 (s, 1H).

### Step 2: 4-benzyloxy-2-chloro-quinoline-7-carbonitrile (Intermediate A-15)

To a suspension of sodium hydride (84 mg of 60% w/w, 2.100 mmol) in DMF (5 mL) was added a solution of 2,4-dichloroquinoline-7-carbonitrile (300 mg, 1.34 mmol) and benzyl alcohol (196 mg, 1.81 mmol) dropwise at 0° C. The reaction mixture was stirred overnight and quenched with water. The aqueous layer was extracted with ethyl acetate (3×15 mL). The organic layer was concentrated and was washed with methanol to obtain 2-benzyloxy-4-chloro-quinoline-7-carbonitrile (190 mg, 48%). ESI-MS m/z calc. 294.05, found 295.1 (M+1)⁺. ¹H NMR (400 MHz, CDCl₃) δ (ppm) 8.24-8.18 (m, 2H), 7.63 (dd, J=8.5, 1.6 Hz, 1H), 7.53-7.48 (m, 2H), 7.43-7.34 (m, 3H), 7.21 (s, 1H), 5.54 (s, 2H). The filtrate was evaporated and purified by preparative reverse phase HPLC (C₁₈) using 1 to 99% ACN in water (HCl modifier) to give 4-benzyloxy-2-chloro-quinoline-7-carbonitrile (Intermediate A-15, 34.7 mg, 9%). ESI-MS m/z calc. 294.05, found 295.2 (M+1)⁺. ¹H NMR (400 MHz, DMSO-d₆) δ (ppm) 8.32-8.27 (m, 2H), 7.66 (dd, J=8.6, 1.4 Hz, 1H), 7.52-7.40 (m, 5H), 6.94 (s, 1H), 5.31 (s, 2H).

### Intermediate A-16

### 4-benzyloxy-2-chloro-5-methoxy-1,7-naphthyridine

### Step 1: 4-hydroxy-5-methoxy-1H-1,7-naphthyridin-2-one

To ethyl 5-methoxy-4-methyl-2-oxo-1H-1,7-naphthyridine-3-carboxylate (4.44 g, 16.93 mmol) in water (66 mL) hydrochloric acid (37% in water) (802.13 g, 22 mL of 37% w/w, 8.14 mol) was added and the reaction mixture was heated to 90° C. overnight. The reaction mixture was cooled to room temperature over 30 minutes and concentrated under reduced pressure to afford 4-hydroxy-5-methoxy-1H-1,7-naphthyridin-2-one (3.25 g, 100%) as a yellow solid. ESI-MS m/z calc. 192.05, found 193.06 (M+1)⁺. ¹H-NMR (400 MHz, DMSO-d₆-D6) δ (ppm) 11.75 (s, 1H), 8.43 (s, 1H), 8.18 (s, 1H), 6.16 (s, 1H), 4.00 (s, 3H).

### Step 2: 2,4-dichloro-5-methoxy-1,7-naphthyridine

To phosphorus oxychloride (65.8 g, 40 mL, 429.14 mmol) was added 4-hydroxy-5-methoxy-1H-1,7-naphthyridin-2-one (2.75 g, 14.31 mmol). The reaction mixture was heated at 120° C. for 2 h. The reaction mixture was cooled to room temperature over 1 h and concentrated under reduced pressure. The crude was poured onto ice cooled saturated sodium carbonate solution (50 mL), extracted with DCM (2×50 mL), dried over sodium sulfate and concentrated under reduced pressure to give 2,4-dichloro-5-methoxy-1,7-naphthyridine (3.27 g, 100%) as a dark brown oil. ESI-MS m/z calc. 227.9, found 229.0 (M+1)⁺.

### Step 3: 4-benzyloxy-2-chloro-5-methoxy-1,7-naphthyridine (Intermediate A-16)

To benzyl alcohol (522.50 mg, 0.5 mL, 4.83 mmol) in DMF (20 mL) at 0° C. under argon was added sodium hydride in mineral oil (200 mg, 60% w/w, 5 mmol). The reaction mixture was stirred for 1 h and cooled to −40° C., then 2,4-dichloro-5-methoxy-1,7-naphthyridine (1 g, 4.33 mmol) was added. The reaction mixture was stirred at this temperature for 2 hours, warmed to −20° C. and kept at this temperature for 1 hour and at 0° C. for 30 minutes. The reaction mixture was re-cooled to −10° C., quenched with saturated ammonium chloride solution (10 mL) and stirred for 30 mins. The reaction mixture was partitioned between ethyl acetate (100 mL) and water (50 mL). The ethyl acetate layer was washed with water (2×50 mL) and brine (30 mL), dried over sodium sulfate and concentrated. The crude was triturated with ethyl acetate (20 mL) and the solid was collected by filtration which after drying gave 4-benzyloxy-2-chloro-5-methoxy-1,7-naphthyridine (Intermediate A-16, 650 mg, 49%). ESI-MS m/z calc. 300.06, found 301.02 (M+1)⁺. ¹H-NMR (400 MHz, CDCl₃) δ (ppm) 8.98 (br s, 1H), 8.19 (br s, 1H), 7.53 (d, J=7.3 Hz, 2H), 7.46-7.36 (m, 3H), 6.93 (s, 1H), 5.28 (s, 2H), 4.03 (s, 3H)

### Intermediate A-17

### 4-benzyloxy-2-chloro-pyrido[2,3-d]pyridazin-5-ol

### Step 1: ethyl 2-[(E)-(tert-butoxycarbonylhydrazono)methyl]-4,6-dichloro-pyridine-3-carboxylate

In a reaction vial, ethyl 4,6-dichloro-2-formyl-pyridine-3-carboxylate (2 g, 8.06 mmol) was dissolved in dioxane (30 mL). tert-butyl N-aminocarbamate (1.28 g, 9.69 mmol) was added and the reaction mixture was stirred at room temperature for 16 h. The reaction mixture was evaporated to dryness and purified by silica gel column chromatography using 0 to 50% ethyl acetate in hexanes gradient to obtain ethyl 2-[(E)-(tert-butoxycarbonylhydrazono)methyl]-4,6-dichloro-pyridine-3-carboxylate (2.9 g, 99%) as a white solid. ESI-MS m/z calc. 361.06, found 362.2 (M+1)⁺.

### Step 2: 2,4-dichloro-6H-pyrido[2,3-d]pyridazin-5-one

ethyl 2-[(E)-(tert-butoxycarbonylhydrazono)methyl]-4,6-dichloro-pyridine-3-carboxylate (765 mg, 2.11 mmol) in DCM (7 mL) was mixed with TFA (1 mL, 12.98 mmol). The reaction mixture was stirred at room temperature for 4 hours. The solvent was evaporated and the crude material was purified via silica gel column chromatography using 0 to 20% ethyl acetate in hexanes to obtain 2,4-dichloro-6H-pyrido[2,3-d]pyridazin-5-one (361 mg, 79%). ESI-MS m/z calc. 214.96, found 216.0 (M+1)⁺. ¹H NMR (400 MHz, DMSO-d₆) δ (ppm) 13.06 (s, 1H), 8.33 (s, 1H), 8.14 (s, 1H).

### Step 3: 4-benzyloxy-2-chloro-pyrido[2,3-d]pyridazin-5-ol (Intermediate A-17)

A round bottom flask equipped with a stir bar was charged with THF (1.5 mL), benzyl alcohol (178 μL, 1.72 mmol) was added followed by the addition of sodium hydride (90 mg of 60% w/w, 2.25 mmol) and the reaction mixture was stirred for 30 minutes. The reaction mixture was then cooled to −40° C. and 2,4-dichloro-6H-pyrido[2,3-d]pyridazin-5-one (370 mg, 1.71 mmol) was added as a solution in THF (500 μL) and DMF (1 mL). The reaction mixture was warmed to room temperature and stirred for 90 minutes. The reaction mixture was quenched with water and the aqueous layer was extracted with ethyl acetate (3×). The combined organic layer was washed with brine (2×), dried over magnesium sulfate, filtered and concentrated. The crude material was purified via silica gel column chromatography using 0 to 50% ethyl acetate in hexanes to obtain 4-benzyloxy-2-chloro-pyrido[2,3-d]pyridazin-5-ol (Intermediate A-17, 80 mg, 16%) ESI-MS m/z calc. 287.05, found 288.1 (M+1)⁺.

### Intermediate A-18

### 4-chloro-2-methoxy-6H-pyrido[2,3-d]pyridazin-5-one

### Step 1: 4-chloro-2-methoxy-6H-pyrido[2,3-d]pyridazin-5-one (Intermediate A-18)

To a suspension of 2,4-dichloro-6H-pyrido[2,3-d]pyridazin-5-one (600 mg, 2.77 mmol) in methanol (18 mL) was added sodium methoxide (25% w/w in methanol) (600 mg, 25% w/w, 2.77 mmol) and the mixture stirred at room temperature overnight. The solvent was evaporated under reduced pressure to give a mixture of 4-chloro-2-methoxy-6H-pyrido[2,3-d]pyridazin-5-one (Intermediate A-18, 774 mg, 46%). ¹H-NMR (400 MHz, DMSO-d6) δ 8.13 (s, 1H), 7.34 (s, 1H), 3.98 (s, 3H). ESI-MS m/z calc. 211.01, found 212.02 (M+1)⁺ and 2-chloro-4-methoxy-6H-pyrido[2,3-d]pyridazin-5-one (774 mg, 46%) ¹H-NMR (400 MHz, DMSO-d6) δ 8.15 (s, 1H), 7.41 (s, 1H), 3.98 (s, 3H). ESI-MS m/z calc. 211.01, found 212.0 (M+1)⁺.

### Intermediates A-19 to A-36

### General Procedure for Synthesis of Intermediate A-19 to A-36

Intermediates A-19 to A-36 (see Table 1) were prepared using the appropriate dichloro-pyridine and procedure analogous to that found in Intermediate A-1, Step 1. Dichloro-pyridines were obtained from commercial sources. Benzyl alcohol or 2-Methoxy benzyl alcohol can be used. DMF, THF or 2-Me THF can be used as the appropriate solvents.

TABLE 1

| | | LC/MS | |
|---|---|---|---|
| | | (m/z calc.); | |
| Intermediate | Compound Name | Found [M + H]⁺ | NMR (shifts in ppm) |
| Intermediate A-19 | | 301.07; 302.3 | ¹H NMR (400 MHz, DMSO-d₆) δ (ppm) 8.08 (dd, J = 9.2, 6.1 Hz, 1H), 7.73 (dd, J = 10.2, 2.5 Hz, 1H), 7.55 (td, J = 8.5, 2.5 Hz, 1H), 7.54-7.50 (m, 2H), 7.47-7.39 (m, 3H), 5.18 (s, 2H), 2.38 (s, 3H). ¹⁹F NMR (376 MHz, DMSO-d₆) δ (ppm) −109.14 |
| | 4-benzyloxy-2-chloro-7- | | (td, J = 9.3, 6.0 Hz, 1F). |
| | fluoro-3-methyl- | | |
| | quinoline | | |
| Intermediate A-20 | | 301.07 302.3 | ¹H NMR (400 MHz, DMSO-d₆) δ (ppm) 8.01 (dd, J = 9.1, 5.2 Hz, 1H), 7.68 (td, J = 8.7, 2.9 Hz, 1H), 7.64 (dd, J = 9.6, 2.9 Hz, 1H), 7.55-7.50 (m, 2H), 7.47-7.38 (m, 3H), 5.18 (s, 2H), 2.39 (s, 3H). ¹⁹F NMR (376 MHz, DMSO-d₆) δ (ppm) −111.96 |
| | 4-benzyloxy-2-chloro-6- | | (td, J = 8.8, 5.0 Hz, 1F). |
| | fluoro-3-methyl- | | |
| | quinoline | | |
| Intermediate A-21 | | 283.08 284.3 | ¹H NMR (400 MHz, DMSO-d₆) δ (ppm) 8.04 (dd, J = 8.4, 1.4 Hz, 1H), 7.94 (dd, J = 8.4, 1.7 Hz, 1H), 7.78 (ddd, J = 8.4, 6.9, 1.5 Hz, 1H), 7.63 (ddd, J = 8.2, 6.8, 1.2 Hz, 1H), 7.57- 7.50 (m, 2H), 7.48-7.38 (m, 3H), 5.17 (s, 2H), 2.39 (s, 3H). |
| | 4-benzyloxy-2-chloro-3- | | |
| | methyl-quinoline | | |
| Intermediate A-22 | | 283.08 284.3 | ¹H NMR (400 MHz, DMSO-d₆) δ (ppm) 7.89 (s, 1H), 7.78 (d, J = 8.5 Hz, 1H), 7.63 (dd, J = 8.6, 2.0 Hz, 1H), 7.60-7.54 (m, 2H), 7.50-7.43 (m, 2H), 7.43-7.36 (m, 1H), 7.17 (s, 1H), 5.42 (s, 2H), 2.48 (s, 3H). |
| | 4-benzyloxy-2-chloro-6- | | |
| | methyl-quinoline | | |
| Intermediate A-23 | | 313.09 314.3 | — |
| | 2-chloro-4-[(4- | | |
| | methoxyphenyl)methoxy]- | | |
| | 8-methyl-quinoline | | |
| Intermediate A-24 | | 299.07 300.3 | ¹H NMR (400 MHz, DMSO-d₆) δ (ppm) 7.81 (d, J = 9.1 Hz, 1H), 7.59- 7.54 (m, 2H), 7.49-7.42 (m, 3H), 7.42-7.36 (m, 2H), 7.17 (s, 1H), 5.46 (s, 2H), 3.88 (s, 3H). |
| | 4-benzyloxy-2-chloro-6- | | |
| | methoxy-quinoline | | |
| Intermediate A-25 | | 299.07 301.2 | ¹H NMR (400 MHz, CDCl₃) δ (ppm) 7.62-7.52 (m, 4H), 7.44 (t, J = 7.4 Hz, 2H), 7.38 (d, J = 7.5 Hz, 1H), 6.89 (dd, J = 7.7, 1.4 Hz, 1H), 6.79 (s, 1H), 5.27 (s, 2H), 1.56 (s, 3H). |
| | 4-benzyloxy-2-chloro-5- | | |
| | methoxy-quinoline | | |
| Intermediate A-26 | | 287.05 288.1 | ¹H NMR (400 MHz, CDCl₃) δ (ppm) 8.04-7.91 (m, 1H), 7.55-7.36 (m, 7H), 6.88 (s, 1H), 5.29 (s, 2H). |
| | 4-benzyloxy-2-chloro-8- | | |
| | fluoro-quinoline | | |
| Intermediate A-27 | | 287.05 288.1 | ¹H NMR (400 MHz, CDCl₃) δ (ppm) 8.20 (dd, J = 9.2, 6.0 Hz, 1H), 7.58 (dd, J = 9.9, 2.3 Hz, 1H), 7.52-7.39 (m, 5H), 7.30-7.23 (m, 1H), 6.81 (s, 1H), 5.28 (s, 2H). ¹⁹F NMR (377 MHz, CDC1₃) δ (ppm) −107.64- −108.00 (m, 1F). |
| | 4-benzyloxy-chloro-7- | | |
| | fluoro-quinoline | | |
| Intermediate A-28 | | 287.05 288.1 | ¹H NMR (400 MHz, CDCl₃) δ (ppm) 7.94 (dd, J = 9.0, 5.1 Hz, 1H), 7.78 (dd, J = 9.2, 2.8 Hz, 1H), 7.53-7.38 (m, 6H), 6.83 (s, 1H), 5.27 (s, 2H). ¹⁹F NMR (377 MHz, CDCl₃) δ (ppm) −112.82-−113.06 (m, 1F). |
| | 4-benzyloxy-2-chloro-6- | | |
| | fluoro-quinoline | | |
| Intermediate A-29 | | 317.06 316.0 (M − 1)⁻ | ¹H NMR (400 MHz, CDCl₃) δ (ppm) 7.76 (d, 1H, J = 8.4 Hz), 7.63 (td, 1H, J = 8.2, 5.4 Hz), 7.47 (dd, 2H, J = 11.5, 2.8 Hz), 7.17 (ddd, 1H, J = 11.7, 7.9, 1.0 Hz), 7.00-6.97 (m, 2H), 6.86 (s, 1H), 5.23 (s, 2H), 3.86 (s, 3H). ¹⁹F- NMR (376 MHz, CDCl₃) δ (ppm)- |
| | 2-chloro-5-fluoro-4-[(4- | | 110.1 (s, 1F). |
| | methoxyphenyl)methoxy] | | |
| | quinoline | | |
| Intermediate A-30 | | 270.06 271.0 | ¹H NMR (400 MHz, CDCl₃) δ (ppm) 8.97 (dd, J = 4.2, 1.5 Hz, 1H), 8.26 (dd, J = 8.6, 1.5 Hz, 1H), 7.66 (dd, J = 8.6, 4.2 Hz, 1H), 7.55-7.48 (m, 2H), 7.45-7.33 (m, 3H), 6.98 (s, 1H), 5.44 (s, 2H). |
| | 4-benzyloxy-2-chloro- | | |
| | 1,5-naphthyridine | | |
| Intermediate A-31 | | 270.06 271.1 | ¹H NMR (400 MHz, CDCl₃) δ (ppm) 9.06 (dd, J = 4.4, 2.0 Hz, 1H), 8.55 (dd, J = 8.3, 2.0 Hz, 1H), 7.54-7.38 (m, 6H), 6.91 (s, 1H), 5.29 (s, 2H). |
| | 4-benzyloxy-2-chloro- | | |
| | 1,8-naphthyridine | | |
| Intermediate A-32 | | 300.07 301.1 | — |
| | 4-benzyloxy-2-chloro-6- | | |
| | methoxy-1,5- | | |
| | naphthyridine | | |
| Intermediate A-33 | | 337.05 338.3 | ¹H NMR (400 MHz, DMSO-d₆) δ (ppm) 8.38 (s, 1H), 8.12-8.04 (m, 2H), 7.61-7.56 (m, 2H), 7.51-7.45 (m, 2H), 7.44-7.41 (m, 1H), 7.41 (s, 1H), 5.50 (s, 2H). |
| | 4-benzyloxy-2-chloro-6- | | |
| | (trifluoromethyl)quinoline | | |
| Intermediate A-34 | | 305.05 306.2 | ¹H NMR (400 MHz, DMSO-d₆) δ (ppm) 8.00 (dd, J = 11.0, 8.7 Hz, 1H), 7.96 (dd, J = 11.6, 7.6 Hz, 1H), 7.61- 7.55 (m, 2H), 7.49-7.43 (m, 2H), 7.43-7.37 (m, 1H), 7.30 (s, 1H), 5.46 (s, 2H). |
| | 4-benzyloxy-2-chloro- | | |
| | 6,7-difluoro-quinoline | | |
| Intermediate A-35 | | 285.06 286.05 | — |
| | 4-benzyloxy-2-chloro- | | |
| | quinolin-5-ol | | |
| Intermediate A-36 | | 342.07 343.02 | ¹H NMR (400 MHz, CDCl₃) δ (ppm) (s, 1H), 8.79 (d, J = 5.8 Hz, 1H), 7.75 (d, J = 5.8 Hz, 1H), 7.51-7.33 (m, 5H), 5.38 (s, 2H), 4.50 (q, J = 7.2 Hz, 2H), 1.44 (t, J = 7.3 Hz, 3H). |
| | ethyl 4-benzyloxy-2- | | |
| | chloro-1,6-naphthyridine- | | |
| | 3-carboxylate | | |

### Intermediate A-37

### 2-chloro-4-[(4-methoxyphenyl)methoxy]-1,6-naphthyridine-5-carbonitrile

### Step 1: 2-chloro-4-[(4-methoxyphenyl)methoxy]-1,6-naphthyridine

To a mixture at 0° C. of 2,4-dichloro-1,6-naphthyridine (3.29 g, 16.5 mmol) and (4-methoxyphenyl)methanol (2.28 g, 2.05 mL, 16.47 mmol) in DMF (33 mL) and 2-MeTHF (33 mL) was added sodium hydride (715 mg, 60% in mineral oil, 17.88 mmol) portionwise. The mixture was stirred at 0° C. for 1 h, then gradually warmed to room temperature and stirred for 19.5 h. The mixture was poured into a stirring mixture of 0.1 M aqueous HCl (100 mL) and 2-MeTHF (100 mL). The layers were separated and the aqueous layer extracted with additional 2-MeTHF (2×100 mL). The organic layers were combined and washed with water (2×50 mL), 1:1 water/brine (50 mL) and brine (50 mL). The solution was dried over sodium sulfate, filtered, and concentrated under reduced pressure. Purification by silica gel chromatography (5-100% ethyl acetate/heptanes) provided 2-chloro-4-[(4-methoxyphenyl)methoxy]-1,6-naphthyridine (1.95 g, 39%) as a pale yellow solid. ESI-MS m/z calc. 300.06, found 301.2 (M+1)⁺. ¹H NMR (400 MHz, CDCl₃) δ (ppm) 9.44 (s, 1H), 8.74 (d, J=5.9 Hz, 1H), 7.72-7.67 (m, 1H), 7.48-7.42 (m, 2H), 7.09 (s, 1H), 6.97-6.88 (m, 2H), 5.50 (s, 2H), 3.83 (s, 3H). ESI-MS m/z calc. 300.06, found 301.2 (M+1)⁺.

### Step 2: 2-chloro-4-[(4-methoxyphenyl)methoxy]-6-oxido-1,6-naphthyridin-6-ium

A solution of 2-chloro-4-[(4-methoxyphenyl)methoxy]-1,6-naphthyridine (5 g, 16.6 mmol) in DCM (100 mL) was cooled to 0° C. and treated with solid 3-chlorobenzenecarboperoxoic acid (4.7 g, 21 mmol). The reaction was warmed to room temperature and stirred for 4 h. The mixture was quenched with saturated aqueous sodium bicarbonate (100 mL) and the layers were separated. The aqueous layer was extracted with additional DCM (3×25 mL). The combined organic layers were washed with brine (20 mL×2), dried with anhydrous magnesium sulfate, filtered, and concentrated in vacuo to obtain 2-chloro-4-[(4-methoxyphenyl)methoxy]-6-oxido-1,6-naphthyridin-6-ium (5.1 g, 97%). ESI-MS m/z calc. 316.06, found 317.2 (M+1)⁺. ¹H NMR (400 MHz, DMSO-d₆) δ (ppm) 8.66 (d, J=2.1 Hz, 1H), 8.40 (dd, J=7.3, 2.1 Hz, 1H), 7.86 (d, J=7.3 Hz, 1H), 7.53-7.47 (m, 2H), 7.41 (s, 1H), 7.05-6.95 (m, 2H), 5.39 (s, 2H), 3.78 (s, 3H).

### Step 3: 2-chloro-4-[(4-methoxyphenyl)methoxy]-1,6-naphthyridine-5-carbonitrile

To a solution of 2-chloro-4-[(4-methoxyphenyl)methoxy]-6-oxido-1,6-naphthyridin-6-ium (6.1 g, 19.3 mmol) in DCM (50 mL) under an atmosphere of nitrogen was added trimethylsilyl cyanide (6.8 mL, 51 mmol), followed by the addition of TEA (8 mL, 57.4 mmol). The mixture was stirred for 20 h at room temperature. The reaction was quenched with water and the aqueous layer was extracted with DCM (3×). The combined organic layers were dried over magnesium sulfate, filtered and concentrated. Purification by silica gel chromatography (0-100% ethyl acetate/DCM) provided 2-chloro-4-[(4-methoxyphenyl)methoxy]-1,6-naphthyridine-5-carbonitrile (5.5 g, 88%). ¹H NMR (400 MHz, DMSO-d₆) δ 8.90 (d, J=5.8 Hz, 1H), 8.11 (d, J=5.7 Hz, 1H), 7.61 (s, 1H), 7.60-7.52 (m, 2H), 7.03-6.95 (m, 2H), 5.52 (s, 2H), 3.77 (s, 3H).

### Example 2—Preparation of Intermediates B-1 to B-98

### Intermediate B-1

### 4,4,5,5-tetramethyl-2-[2-methyl-4-[1-(trifluoromethyl)cyclopropyl]phenyl]-1,3,2-dioxaborolane

### Step 1: 1-bromo-2-methyl-4-[1-(trifluoromethyl)cyclopropyl]benzene

Under an inert atmosphere, acetic acid (10 mL) was placed in a 50 mL flask and 1-methyl-3-[1-(trifluoromethyl)cyclopropyl]benzene (1 g, 5 mmol) was added. Then, bromine (310 μL, 6 mmol) was added and the reaction mixture was stirred for 18 hours at 15˜20° C. The reaction mixture was added onto ice and stirred for 20 minutes. Ethyl acetate was added, and the layers were separated. The organic layer was washed with sodium bicarbonate (3×), followed by washing with water (2×). The organic layer was dried over magnesium sulfate, filtered, and concentrated. The crude material was purified via silica gel column chromatography using 0 to 5% ethyl acetate in hexanes to obtain 1-bromo-2-methyl-4-[1-(trifluoromethyl)cyclopropyl]benzene (1.12 g, 80%). ¹H NMR (400 MHz, CDCl₃) δ (ppm) 7.49 (d, J=8.2 Hz, 1H), 7.32 (d, J=2.2 Hz, 1H), 7.13 (dd, J=8.3, 2.3 Hz, 1H), 2.39 (s, 3H), 1.36-1.31 (m, 2H), 1.03-0.96 (m, 2H).

### Step 2: 4,4,5,5-tetramethyl-2-[2-methyl-4-[1-(trifluoromethyl)cyclopropyl]phenyl]-1,3,2-dioxaborolane (Intermediate B-1)

A microwave vial charged with 1-bromo-2-methyl-4-[1-(trifluoromethyl)cyclopropyl]benzene (3.1 g, 11.11 mmol), bis(pinacolato)diboron (8.7 g, 34.26 mmol), potassium acetate (2.3 g, 23.20 mmol), Pd(dppf)₂Cl₂·DCM (910 mg, 1.11 mmol) and dioxane (30 mL) was degassed under nitrogen, sealed and heated at 90° C. for 16 h. The reaction mixture was filtered through a plug of celite, the solvent was evaporated and the crude material was purified via silica gel column chromatography using 0 to 10% ethyl acetate in hexanes to obtain 4,4,5,5-tetramethyl-2-[2-methyl-4-[1-(trifluoromethyl)cyclopropyl]phenyl]-1,3,2-dioxaborolane (Intermediate B-1, 2.86 g, 79%) as a pale yellow solid. ¹H NMR (400 MHz, CDCl₃) δ (ppm) 7.74 (d, J=8.1 Hz, 1H), 7.26-7.24 (m, 2H), 2.54 (s, 3H), 1.36-1.30 (m, 14H), 1.04-0.98 (m, 2H).

### Intermediate B-2

### 2-(4-tert-butyl-5-chloro-2-methyl-phenyl)-4,4,5,5-tetramethyl-1,3,2-dioxaborolane

### Step 1: 1-(4-bromo-2-chloro-5-methyl-phenyl)ethan-1-one

To a microwave vial containing a solution of 1-bromo-5-chloro-4-iodo-2-methyl-benzene (21 g, 63.37 mmol) and tributyl(1-ethoxyvinyl)stannane (21.4 mL, 63.34 mmol) in dioxane (105 mL) was added PdCl₂(PPh₃)₂(2.25 g, 3.21 mmol). The reaction mixture was degassed with nitrogen for 30-60 seconds, sealed and heated at 100° C. for 17 hours. The reaction mixture cooled to room temperature and quenched with water. The aqueous layer was extracted with DCM (3×). The combined organic layer was dried over magnesium sulfate, filtered and concentrated to obtain 1-bromo-5-chloro-4-(1-ethoxyvinyl)-2-methyl-benzene. ESI-MS m/z calc. 273.98, found 277.07 (M+2)⁺. The intermediate was taken up in THF (100 mL) and HCl (95 mL of 1 M, 95 mmol) was added, and the reaction mixture was stirred at room temperature for 1 h. The aqueous layer was extracted with DCM (3×), dried over magnesium sulfate, filtered, and concentrated. The crude material was purified via silica gel column chromatography using 0 to 15% ethyl acetate in Hexanes to obtain 1-(4-bromo-2-chloro-5-methyl-phenyl)ethan-1-one (12.62 g, 80%) as a white solid. ESI-MS m/z calc. 245.94, found 248.95 (M+3)⁺. ¹H NMR (400 MHz, DMSO-d₆) δ (ppm) 7.83 (s, 1H), 7.74 (s, 1H), 2.58 (s, 3H), 2.38 (s, 3H).

### Step 2: 1-bromo-4-tert-butyl-5-chloro-2-methyl-benzene

To a solution of tetrachlorotitanium in toluene (50 mL of 1 M, 50 mmol) in DCM (35 mL) at −40° C. was added dimethylzinc in toluene (33 mL of 2 M, 66 mmol) slowly maintaining the temperature below −40° C. (internal temperature). The reaction mixture was stirred at −40° C. for 30 min and then a solution of 1-(4-bromo-2-chloro-5-methyl-phenyl)ethanone (6.3 g, 25.45 mmol) in DCM (10 mL) was added dropwise, maintaining the internal temperature below −40° C. The reaction mixture was gradually warmed to room temperature and stirred for 2 h. The reaction mixture was quenched by slowly pouring it into ice and saturated sodium bicarbonate solution. The aqueous phase was acidified with concentrated HCl and then extracted with DCM. The organic phases were combined, dried over magnesium sulfate, filtered, and concentrated in vacuo. The crude material was purified via silica gel column chromatography using hexanes to obtain 1-bromo-4-tert-butyl-5-chloro-2-methyl-benzene (5.8 g, 87%). ¹H NMR (400 MHz, DMSO-d₆) δ (ppm) 7.60 (s, 1H), 7.42 (s, 1H), 2.33 (s, 3H), 1.41 (s, 9H).

### Step 3: 2-(4-tert-butyl-5-chloro-2-methyl-phenyl)-4,4,5,5-tetramethyl-1,3,2-dioxaborolane (Intermediate B-2)

A solution of 1-bromo-4-tert-butyl-5-chloro-2-methyl-benzene (4.49 g, 17.2 mmol) in 1,4-dioxane (50 mL) was sparged with nitrogen for 20 min then bis(pinacolato)diboron (5.7 g, 22.5 mmol), potassium acetate (5.0 g, 51 mmol) and PdCl₂(dppf).DCM (1.4 g, 1.7 mmol) were added. The mixture was stirred for 4 h at 100° C. The mixture was cooled to room temperature and additional bis(pinacolato)diboron (1.9 g, 7.5 mmol) and PdCl₂(dppf).DCM (500 mg, 0.612 mmol) added. The mixture was sparged with nitrogen for 5 min then stirred at 105° C. for 2 h. Once cooled to room temperature, MTBE (50 mL) was added and the mixture was filtered through Celite®. The filtrate was concentrated under reduced pressure and purified by silica gel chromatography using 0 to 5% ethyl acetate in heptanes. The resulting material was triturated in methanol (15 mL) and filtered. The solid was rinsed with cold methanol (5 mL) and dried under vacuum to provide 2-(4-tert-butyl-5-chloro-2-methyl-phenyl)-4,4,5,5-tetramethyl-1,3,2-dioxaborolane (Intermediate B-2, 3.75 g, 71%) as a white solid. ¹H NMR (400 MHz, CDCl₃) δ (ppm) 7.71 (s, 1H), 7.20 (s, 1H), 2.49 (s, 3H), 1.46 (s, 9H), 1.33 (s, 12H). ESI-MS m/z calc. 308.17, found 309.2 (M+1)⁺.

### Intermediate B-3

### 2-[2,5-dimethyl-4-(2,2,2-trifluoro-1,1-dimethyl-ethyl)phenyl]-4,4,5,5-tetramethyl-1,3,2-dioxaborolane

### Step 1:1-4-bromo-2,5-dimethyl-phenyl)-2,2,2-trifluoro-ethanone

To a solution of 1,4-dibromo-2,5-dimethyl-benzene (9.5 g, 36 mmol) in tetrahydrofuran (180 mL) cooled to −78° C. under an atmosphere of nitrogen was added n-BuLi (2.5 M in hexanes) (16 mL of 2.5 M, 40 mmol) dropwise over 40 minutes. The mixture was stirred at −74° C. for 40 minutes. Ethyl 2,2,2-trifluoroacetate (5.73 g, 4.8 mL, 40.34 mmol) was added dropwise over 15 minutes and the reaction mixture was stirred for an additional 30 minutes at −74° C. The reaction mixture was carefully quenched by dropwise addition of a mixture of hydrochloric acid (8.9 mL of 37% w/v, 90 mmol) and ethanol (6 mL) precooled to −78° C. After stirring for 20 minutes, the reaction mixture was warmed to room temperature. The reaction mixture was diluted with water (100 mL). The organic layer was separated, dried over magnesium sulfate, filtered, and concentrated under reduced pressure. The crude product was purified using silica gel chromatography using 100% heptanes to afford 1-(4-bromo-2,5-dimethyl-phenyl)-2,2,2-trifluoro-ethanone (8.55 g, 79%) as a colorless oil. ¹H NMR (400 MHz, CDCl₃) δ (ppm) 7.70 (s, 1H), 7.57 (s, 1H), 2.52 (s, 3H), 2.46 (s, 3H). ¹⁹F NMR (377 MHz, CDCl₃) δ (ppm) −71.21 (s, 3F).

### Step 2: 2-(4-bromo-2,5-dimethyl-phenyl)-1,1,1-trifluoro-propan-2-ol

To a solution of 1-(4-bromo-2,5-dimethyl-phenyl)-2,2,2-trifluoro-ethanone (945 mg, 3.36 mmol) in tetrahydrofuran (15 mL) cooled to 0° C. was slowly added a solution of methylmagnesium bromide in diethyl ether (3.3 mL of 3 M, 9.9 mmol) and the mixture was stirred at 50° C. for 1 h. The mixture was cooled to 0° C. and quenched slowly with water, then with a saturated solution of ammonium chloride. The organic layer was separated, and the aqueous layer was extracted with ethyl acetate (3×25 mL). The combined organic layer was washed with brine, dried over anhydrous magnesium sulfate, filtered, concentrated in vacuo to provide the crude product. Purification by silica gel chromatography using 0 to 10% ethyl acetate in heptanes provided 2-(4-bromo-2,5-dimethyl-phenyl)-1,1,1-trifluoro-propan-2-ol (812 mg, 78%). ESI-MS m/z calc. 296, found 279.0 (M−17)⁺. ¹H NMR (400 MHz, CDCl₃) δ (ppm) 7.39 (s, 1H), 7.29 (s, 1H), 2.54 (s, 3H), 2.38 (s, 3H), 2.29 (s, 1H) 1.87-1.84 (m, 3H). ¹⁹F NMR (377 MHz, CDCl₃) δ (ppm) −80.01 (s, 3F).

### Step 3: [1-(4-bromo-2,5-dimethyl-phenyl)-2,2,2-trifluoro-1-methyl-ethyl] methanesulfonate

A solution of 2-(4-bromo-2,5-dimethyl-phenyl)-1,1,1-trifluoro-propan-2-ol (2 g, 6.32 mmol) in tetrahydrofuran (6 mL) was added dropwise at room temperature to a suspension of sodium hydride in mineral oil (770 mg, 60% w/w, 19.25 mmol) in tetrahydrofuran (12 mL). The reaction mixture was stirred at 40° C. for 90 minutes. The reaction mixture was cooled to room temperature and a solution of methanesulfonyl chloride (2.22 g, 1.5 mL, 19.38 mmol) in tetrahydrofuran (12 mL) was added dropwise. The reaction mixture was heated at 40° C. and stirred for 90 minutes. After cooling to room temperature, the reaction mixture was quenched with water (20 mL) and saturated aqueous sodium bicarbonate solution (30 mL). The aqueous layer was extracted with ethyl acetate (2×50 mL). The combined organic layer was washed with brine, dried over anhydrous magnesium sulfate, filtered, and concentrated in vacuo. The crude product was partitioned between acetonitrile (100 mL) and heptanes (100 mL) and the heptane layer was extracted with acetonitrile (50 mL). The combined acetonitrile layers were concentrated under reduced pressure to give crude [1-(4-bromo-2,5-dimethyl-phenyl)-2,2,2-trifluoro-1-methyl-ethyl] methanesulfonate (2.75 g, 96%). ESI-MS m/z calc. 373.98, found 279.2 (M−95)⁺. ¹H NMR (400 MHz, CDCl₃) δ (ppm) 7.43 (s, 1H), 7.27 (s, 1H), 3.17 (s, 3H), 2.55 (s, 3H), 2.39 (s, 3H), 2.35 (d, J=0.7 Hz, 3H). ¹⁹F NMR (377 MHz, CDCl₃) δ (ppm) −79.08 (s, 3F).

### Step 4: 1-bromo-2,5-dimethyl-4-(2,2,2-trifluoro-1,1-dimethyl-ethyl)benzene

To a solution of [1-(4-bromo-2,5-dimethyl-phenyl)-2,2,2-trifluoro-1-methyl-ethyl]methanesulfonate (2.75 g, 6.08 mmol) in DCM (40 mL) cooled at 0° C. was added dropwise a solution of trimethylaluminium (2 M in hexanes) (8.2 mL of 2 M, 16.4 mmol) over a period of 5 min. The reaction mixture was gradually warmed to room temperature and stirred for 2 h. The reaction mixture was slowly quenched with saturated sodium bicarbonate solution (60 mL) and partitioned between brine (60 mL) and DCM (60 mL). The layers were separated, and aqueous layer was extracted with DCM (2×100 mL). The combined organic layers were washed with brine (50 mL), dried over anhydrous sodium sulfate, filtered, and concentrated under reduced pressure. The crude product was purified by silica gel flash chromatography using heptanes to afford 1-bromo-2,5-dimethyl-4-(2,2,2-trifluoro-1,1-dimethyl-ethyl)benzene (1.48 g, 79%) as a yellow oil. ¹H NMR (400 MHz, CDCl₃) δ (ppm) 7.35 (s, 1H), 7.31 (s, 1H), 2.50 (s, 3H), 2.38 (s, 3H), 1.67 (s, 6H). ¹⁹F NMR (377 MHz, CDCl₃) δ (ppm) −75.18 (s, 3F).

### Step 5: 2-[2,5-dimethyl-4-(2,2,2-trifluoro-1,1-dimethyl-ethyl)phenyl]-4,4,5,5-tetramethyl-1,3,2-dioxaborolane (Intermediate B-3)

2-[2,5-dimethyl-4-(2,2,2-trifluoro-1,1-dimethyl-ethyl)phenyl]-4,4,5,5-tetramethyl-1,3,2-dioxaborolane (Intermediate B-3) was prepared from 1-bromo-2,5-dimethyl-4-(2,2,2-trifluoro-1,1-dimethyl-ethyl)benzene using procedure analogous to that found in Intermediate B-1, Step 2. ESI-MS m/z calc. 342.19, found 343.2 (M+1)⁺. ¹H NMR (400 MHz, DMSO-d₆) δ (ppm) 7.42 (s, 1H), 7.28 (s, 1H), 2.45 (s, 3H), 2.42 (s, 3H), 1.65 (s, 6H), 1.29 (s, 12H). ¹⁹F NMR (377 MHz, DMSO-d₆) δ (ppm) −73.86 (s, 3F).

### Intermediate B-4

### 2-[5-chloro-2-methyl-4-(2,2,2-trifluoro-1,1-dimethyl-ethyl)phenyl]-4,4,5,5-tetramethyl-1,3,2-dioxaborolane

### Step 1: 1-(4-bromo-2-chloro-5-methyl-phenyl)-2,2,2-trifluoro-ethanone

To a stirring solution of 1,4-dibromo-2-chloro-1-methyl-benzene (10.0 g, 35.2 mmol) in diethyl ether (200 mL) was added a solution of n-BuLi (14 mL of 2.5 M in hexanes, 35 mmol) at −78° C. The mixture was kept at this temperature for 40 min then a solution of methyl trifluoroacetate (4.5 g, 3.5 mL, 35 mmol) in diethyl ether (25 mL) was added dropwise. The mixture was stirred at this temperature for 40 min. A 3:2 mixture of concentrated hydrochloric acid and ethanol (50 mL) was added dropwise to quench the reaction at −78° C. The aqueous phase was extracted with MTBE (3×150 mL). The combined organic layer was washed with water (3×100 mL) and brine, dried over sodium sulfate, filtered and concentrated under reduced pressure. Purification by silica gel column chromatography using 0 to 10% DCM in heptanes provided 1-(4-bromo-2-chloro-5-methyl-phenyl)-2,2,2-trifluoro-ethanone (8.2 g, 73%) as a clear oil. ¹H NMR (400 MHz, CDCl₃) δ (ppm) 7.76 (s, 1H), 7.57 (s, 1H), 2.46 (s, 3H).

### Step 2: 2-(4-bromo-2-chloro-5-methyl-phenyl)-1,1,1-trifluoro-propan-2-ol

To a stirring solution of 1-(4-bromo-2-chloro-5-methyl-phenyl)-2,2,2-trifluoro-ethanone (1.52 g, 5.04 mmol) in THF (25 mL) was added methylmagnesium bromide (5.0 mL, 3 M in diethyl ether, 15 mmol) dropwise at 0° C. The mixture was heated at 50° C. for 1 h, then cooled to 0° C. and quenched dropwise with water (10 mL) followed by saturated aqueous ammonium chloride (20 mL). The layers were separated and the aqueous layer was extracted with ethyl acetate (3×30 mL). The combined organic layer was washed with brine (50 mL), dried over sodium sulfate, filtered and concentrated under reduced pressure. Purification by silica gel chromatography (40 g silica, 0-5% ethyl acetate/heptanes) provided 2-(4-bromo-2-chloro-5-methyl-phenyl)-1,1,1-trifluoro-propan-2-ol (1.27 g, 77%). ¹H NMR (400 MHz, CDCl₃) δ (ppm) 7.61 (s, 1H), 7.50 (s, 1H), 3.70 (s, 1H), 2.41 (s, 3H), 1.93 (s, 3H). ESI-MS m/z calc. 315.95, found 299.2 (M−17)⁺.

### Step 3: [1-(4-bromo-2-chloro-5-methyl-phenyl)-2,2,2-trifluoro-1-methyl-ethyl]methanesulfonate

To a solution of 2-(4-bromo-2-chloro-5-methyl-phenyl)-1,1,1-trifluoro-propan-2-ol (100 mg, 0.284 mmol) and triethylamine (51 mg, 70 μL, 0.50 mmol) in dichloromethane (2 mL) at 0° C. was added methanesulfonyl chloride (52 mg, 35 μL, 0.45 mmol). The reaction mixture was stirred at room temperature for 18 h. A second portion of methanesulfonyl chloride (52 mg, 35 μL, 0.45 mmol) and triethylamine (51 mg, 70 μL, 0.50 mmol) were added and the mixture was stirred at room temperature for 16 h. The mixture was diluted with dichloromethane (20 mL) and washed with water (15 mL), saturated aqueous sodium bicarbonate (15 mL) and brine (15 mL). The organic layer was dried over sodium sulfate, filtered and concentrated under reduced pressure to provide [1-(4-bromo-2-chloro-5-methyl-phenyl)-2,2,2-trifluoro-1-methyl-ethyl] methanesulfonate (140 mg, 100%) as a yellow oil. ¹H NMR (400 MHz, CDCl₃) δ (ppm) 7.66 (s, 1H), 7.44 (s, 1H), 3.24 (s, 3H), 2.42 (s, 6H). ¹⁹F NMR (377 MHz, CDCl₃) δ (ppm) −78.82 (s, 3F).

### Step 4: 1-bromo-5-chloro-2-methyl-4-(2,2,2-trifluoro-1,1-dimethyl-ethyl)benzene

A stirring solution of [1-(4-bromo-2-chloro-5-methyl-phenyl)-2,2,2-trifluoro-1-methyl-ethyl]methanesulfonate (1.52 g, 3.77 mmol) in dichloromethane (22 mL) at 0° C. was treated dropwise with a solution of trimethylaluminium (5.6 mL of 2 M in toluene, 11 mmol) and stirred at room temperature for 2 h. The mixture was slowly quenched with saturated aqueous sodium bicarbonate (20 mL) and partitioned between brine (20 mL) and DCM (20 mL). The layers were separated and the aqueous layer was extracted with DCM (2×30 mL). The combined organic layers were washed with brine (50 mL), dried over sodium sulfate, filtered and concentrated under reduced pressure. Purification by silica gel chromatography (40 g silica, 0-5% ethyl acetate/heptanes) provided 1-bromo-5-chloro-2-methyl-4-(2,2,2-trifluoro-1,1-dimethyl-ethyl)benzene (928 mg, 75%) as a clear oil. ¹H NMR (400 MHz, CDCl₃) δ (ppm) 7.59 (s, 1H), 7.38 (s, 1H), 2.38 (s, 3H), 1.75 (s, 6H).

### Step 5: 2-[5-chloro-2-methyl-4-(2,2,2-trifluoro-1,1-dimethyl-ethyl)phenyl]-4,4,5,5-tetramethyl-1,3,2-dioxaborolane (Intermediate B-4)

To a stirring solution of 1-bromo-5-chloro-2-methyl-4-(2,2,2-trifluoro-1,1-dimethyl-ethyl)benzene (925 mg, 2.93 mmol) in 1,4-dioxane (5.5 mL) was added bis(pinacolato)diboron (893 mg, 3.52 mmol) and potassium acetate (863 mg, 8.79 mmol). The mixture was degassed with nitrogen for 5 min then PdCl₂(dppf).DCM (240 mg, 0.294 mmol) was added. The mixture was degassed with nitrogen for 5 min then sealed and heated at 120° C. for 15 h. The mixture was cooled to room temperature, filtered through Celite, and the filter cake rinsed with ethyl acetate (20 mL). The filtrate was washed with saturated aqueous ammonium chloride (20 mL) and brine, dried over sodium sulfate, filtered and concentrated under reduced pressure. Purification by silica gel flash chromatography using heptanes provided 2-[5-chloro-2-methyl-4-(2,2,2-trifluoro-1,1-dimethyl-ethyl)phenyl]-4,4,5,5-tetramethyl-1,3,2-dioxaborolane (Intermediate B-4, 769 mg, 69%) as an off-white solid. ¹H NMR (400 MHz, CDCl₃) δ (ppm) 7.77 (s, 1H), 7.32 (s, 1H), 2.50 (s, 3H), 1.75 (s, 6H), 1.34 (s, 12H). ¹⁹F NMR (377 MHz, CDCl₃) δ (ppm) −73.72 (s, 3F). ESI-MS m/z calc. 362.14, found 363.2 (M+1)⁺.

### Intermediate B-5

### 2-[5-fluoro-2-methyl-4-(2,2,2-trifluoro-1,1-dimethyl-ethyl)phenyl]-4,4,5,5-tetramethyl-1,3,2-dioxaborolane

### Step 1: 1-bromo-5-fluoro-2-methyl-4-(2,2,2-trifluoro-1,1-dimethyl-ethyl)benzene

1-bromo-5-fluoro-2-methyl-4-(2,2,2-trifluoro-1,1-dimethyl-ethyl)benzene was prepared from 1,4-dibromo-2-fluoro-5-methyl-benzene using procedure analogous to that found in Intermediate B-4 (Step1 to Step 4), starting with 1,4-dibromo-2-fluoro-5-methyl-benzene. ¹H NMR (400 MHz, CDCl₃) δ (ppm) 7.30-7.23 (m, 2H), 2.38 (s, 3H), 1.63 (s, 6H). ¹⁹F NMR (377 MHz, CDCl₃) δ (ppm) −76.09 (d, J=13.6 Hz, 3F), −108.70-108.90 (m, 1F).

### Step 2: 2-[5-fluoro-2-methyl-4-(2,2,2-trifluoro-1,1-dimethyl-ethyl)phenyl]-4,4,5,5-tetramethyl-1,3,2-dioxaborolane (Intermediate B-5)

2-[5-fluoro-2-methyl-4-(2,2,2-trifluoro-1,1-dimethyl-ethyl)phenyl]-4,4,5,5-tetramethyl-1,3,2-dioxaborolane (Intermediate B-5) was prepared from 1-bromo-5-fluoro-2-methyl-4-(2,2,2-trifluoro-1,1-dimethyl-ethyl)benzene using procedure analogous to that found in Intermediate B-1, Step 2. ¹H NMR (400 MHz, DMSO-d₆) δ (ppm) 7.33 (d, J=7.8 Hz, 1H), 7.29 (d, J=13.4 Hz, 1H), 2.45 (s, 3H), 1.61 (s, 6H), 1.30 (s, 12H). ¹⁹F NMR (377 MHz, DMSO-d₆) δ (ppm) −74.93 (d, J=15.0 Hz, 3F), −113.06-113.23 (m, 1F). ESI-MS m/z calc. 346.17, found 347.2 (M+1)⁺.

### Intermediate B-6

### 2-[4-(3,3-difluorocyclobutyl)-2-methyl-phenyl]-4,4,5,5-tetramethyl-1,3,2-dioxaborolane

### Step 1: 1-bromo-2-methyl-4-vinyl-benzene

To a solution of methyl(triphenyl)phosphonium bromide (14.5 g, 40.59 mmol) in THF (100 mL) was added n-BuLi (16 mL of 2.5 M, 40 mmol) dropwise and stirred for 90 minutes under a stream of nitrogen at 0° C. 4-bromo-3-methyl-benzaldehyde (5 g, 25.12 mmol) in THF (10 mL) was added dropwise at −20° C. and the reaction mixture was stirred for 2 h. The reaction mixture was warmed to room temperature and quenched with saturated ammonium chloride solution. The aqueous layer was extracted with ethyl acetate (3×). The combined organic layer was washed with brine, dried over magnesium sulfate, filtered and concentrated. Purification via silica gel column chromatography using 0 to 30% ethyl acetate in hexanes gave 1-bromo-2-methyl-4-vinyl-benzene (2.1 g, 42%). ¹H NMR (400 MHz, CDCl₃) δ (ppm) 7.47 (d, J=8.2 Hz, 1H), 7.25 (d, J=2.6 Hz, 1H), 7.09 (dd, J=8.2, 2.2 Hz, 1H), 6.63 (dd, J=17.6, 10.8 Hz, 1H), 5.73 (dd, J=17.6, 0.8 Hz, 1H), 5.25 (dd, J=10.9, 0.8 Hz, 1H), 2.39 (s, 3H).

### Step 2: 3-(4-bromo-3-methyl-phenyl)cyclobutanone

To a stirred suspension of activated copper-zinc (3.5 g, 27.14 mmol) and 1-bromo-2-methyl-4-vinyl-benzene (2.1 g, 10.66 mmol) in dry ether (30 mL) was added dropwise through an addition funnel, a solution of 2,2,2-trichloroacetyl chloride (2.4 mL, 21.50 mmol) and POCl₃ (2 mL, 21.46 mmol) in ether (15 mL). The suspension was stirred overnight at reflux. The mixture was cooled to room temperature and filtered through a pad of celite. The filtrate was quenched by slowly pouring into water. The layers were separated, and the organic layer was washed with sodium bicarbonate, dried over magnesium sulfate, filtered and concentrated in vacuo to obtain 3-(4-bromo-3-methyl-phenyl)-2,2-dichloro-cyclobutanone, which was dissolved in acetic acid (7 mL). Zinc (3.15 g, 48.16 mmol) was slowly added portion-wise, and the slurry was stirred at room temperature for 30 minutes followed by heating at 115° C. for 16 hours. The reaction mixture was cooled to room temperature, diluted with ethyl acetate, filtered through celite, and concentrated. The resulting oil was purified by silica gel chromatography by eluting with 0 to 25% ethyl acetate in heptanes followed by a second purification via preparative HPLC (C₁₈) using 1 to 70% ACN in water (HCl modifier) to obtain 3-(4-bromo-3-methyl-phenyl)cyclobutanone (620 mg, 24%) ESI-MS m/z calc. 237.99, found 240.95 (M+3)⁺.

### Step 3: 1-bromo-4-(3,3-difluorocyclobutyl)-2-methyl-benzene

A solution of 3-(4-bromo-3-methyl-phenyl)cyclobutanone (620 mg, 2.59 mmol) in DCM (15 mL) was cooled to −70° C. (external temperature) and DAST (2 mL, 15.14 mmol) was added slowly. The reaction mixture was gradually warmed to room temperature and stirred for 3 days. 1 N sodium hydroxide was added (20 mL) and the reaction mixture was stirred vigorously for 30 minutes. DCM was added and the layers were separated. The organic layer was dried over magnesium sulfate, filtered, and concentrated. Purification via silica gel column chromatography using 0 to 10% ethyl acetate in hexanes afforded 1-bromo-4-(3,3-difluorocyclobutyl)-2-methyl-benzene (400 mg, 59%) ¹H-NMR (400 MHz, CDCl₃) δ (ppm) 7.48 (d, J=8.2 Hz, 1H), 7.09 (d, J=2.3 Hz, 1H), 6.92 (dd, J=8.2, 2.3 Hz, 1H), 3.38-3.24 (m, 1H), 3.05-2.92 (m, 2H), 2.71-2.55 (m, 2H), 2.39 (s, 3H).

### Step 4: 2-[4-(3,3-difluorocyclobutyl)-2-methyl-phenyl]-4,4,5,5-tetramethyl-1,3,2-dioxaborolane (Intermediate B-6)

2-[4-(3,3-difluorocyclobutyl)-2-methyl-phenyl]-4,4,5,5-tetramethyl-1,3,2-dioxaborolane (Intermediate B-6) was prepared from 1-bromo-4-(3,3-difluorocyclobutyl)-2-methyl-benzene using procedure analogous to that found in Intermediate B-1, Step 2 and using Pd(dppf)Cl₂ DCM as a catalyst. ESI-MS m/z calc. 308.18, found 309.17 (M+1)⁺. ¹H NMR (400 MHz, CDCl₃) δ (ppm) 7.75-7.68 (m, 1H), 7.06-6.99 (m, 2H), 3.41-3.27 (m, 1H), 3.05-2.91 (m, 2H), 2.78-2.59 (m, 2H), 2.53 (s, 3H), 1.33 (s, 12H).

### Intermediate B-7

### 2-tert-butyl-3,6-dimethyl-5-(4,4,5,5-tetramethyl-1,3,2-dioxaborolan-2-yl)pyridine

### Step 1: sodium 4,4-dimethyl-1-oxo-pent-2-en-3-olate

To a suspension of sodium hydride in mineral oil (8.4 g, 60% w/w, 210.02 mmol) in diethyl ether (120 mL) at 0° C. was added a solution of 3,3-dimethylbutan-2-one (20.83 g, 26 mL, 207.93 mmol) and ethyl formate (15.47 g, 16.8 mL, 208.87 mmol) in diethyl ether (30 mL) dropwise over 35 minutes. The reaction mixture was gradually warmed to room temperature and stirred overnight. It was diluted with diethyl ether (250 mL) and stirred vigorously for 30 minutes, filtered, rinsed with diethyl ether (2×500 mL) and air dried to give a 3.7:1 crude mixture of Z/E isomer of sodium 4,4-dimethyl-1-oxo-pent-2-en-3-olate (16.05 g, 49%) as an off-white solid. Major isomer: ¹H NMR (400 MHz, DMSO-d₆) δ (ppm) 9.05 (d, J=9.5 Hz, 1H), 4.77 (d, J=9.5 Hz, 1H), 0.94 (s, 9H). Minor isomer: ¹H NMR (400 MHz, DMSO-d₆) δ (ppm) 8.21 (d, J=3.9 Hz, 1H), 4.82 (d, J=3.9 Hz, 1H), 1.01 (s, 9H). ESI-MS m/z calc. 150.06, found 129.0 (M−21)⁺.

### Step 2: 6-tert-butyl-2-hydroxy-pyridine-3-carbonitrile

A solution of piperidine acetate (8.85 g, 60.95 mmol) in water (6.1 mL) was added to a solution of sodium 4,4-dimethyl-1-oxo-pent-2-en-3-olate (16.05 g, 101.55 mmol) and 2-cyanoacetamide (8.54 g, 101.57 mmol) in water (85 mL) at room temperature. The solution was stirred at reflux for 5 h. The reaction mixture was cooled to room temperature and acidified (pH 4) with glacial acetic acid. The resulting precipitate was filtered, rinsed with water (3×500 mL) and dried under high vacuum to give 6-tert-butyl-2-hydroxy-pyridine-3-carbonitrile (10.9 g, 61%) as a light-yellow solid. ESI-MS m/z calc. 176.09, found 177.2 (M+1)⁺. ¹H NMR (400 MHz, DMSO-d₆) δ (ppm) 12.18 (br s, 1H), 8.06 (d, J=7.6 Hz, 1H), 6.24 (br d, J=7.3 Hz, 1H), 1.27 (s, 9H). ESI-MS m/z calc. 176.09, found 177.2 (M+1)⁺.

### Step 3: 5-bromo-6-tert-butyl-2-hydroxy-pyridine-3-carbonitrile

A solution of 6-tert-butyl-2-hydroxy-pyridine-3-carbonitrile (430 mg, 2.44 mmol) and NBS (651 mg, 3.66 mmol) in anhydrous 1,2-dichloroethane (5 mL) was stirred at reflux for 2.5 h. After it was cooled to room temperature, water (10 mL) was added, and the aqueous layer was extracted with DCM (2×10 mL). The combined organic layer was washed with water (15 mL), dried over sodium sulfate, filtered, and concentrated under reduced pressure. The crude product was purified by silica gel flash chromatography using 0 to 10% methanol in DCM to afford 5-bromo-6-tert-butyl-2-hydroxy-pyridine-3-carbonitrile (480 mg, 77%) as a yellow oil. ESI-MS m/z calc. 254.0, found 255.0 (M+1)⁺. ¹H NMR (400 MHz, DMSO-d₆) δ (ppm) 8.37 (s, 1H), 1.45 (s, 9H).

### Step 4: 2,5-dibromo-6-tert-butyl-pyridine-3-carbonitrile

To a stirred suspension of 5-bromo-6-tert-butyl-2-hydroxy-pyridine-3-carbonitrile (16.11 g, 61.76 mmol) in toluene (200 mL) was added phosphorus oxybromide (26.6 g, 92.79 mmol). The reaction mixture was stirred at 95° C. overnight. The reaction mixture was cooled to room temperature and quenched with water (800 mL) and diluted with ethyl acetate (500 mL) and brine (200 mL). The layers were separated, and the aqueous layer was extracted with ethyl acetate (2×400 mL). The combined organic layer was washed with brine (400 mL), dried over sodium sulfate, filtered, and concentrated under reduced pressure. The crude product was purified by silica gel flash chromatography using 0 to 10% ethyl acetate in heptanes to afford 2,5-dibromo-6-tert-butyl-pyridine-3-carbonitrile (15.44 g, 79%) as an orange solid. ESI-MS m/z calc. 254.01, found 255.0 (M+1)⁺.

### Step 5: 6-tert-butyl-2,5-dimethyl-pyridine-3-carbonitrile

A suspension of 2,5-dibromo-6-tert-butyl-pyridine-3-carbonitrile (2 g, 6.28 mmol), trimethylboroxine (2.42 g, 2.7 mL, 19.31 mmol) and potassium carbonate (5.21 g, 37.7 mmol) in anhydrous 1,4-dioxane (20 mL) was purged with nitrogen for 10 min, Pd(dppf)₂Cl₂·DCM (515 mg, 0.63 mmol) was added to the reaction mixture and it was purged with nitrogen for an additional 10 minutes. The reaction mixture was heated at 100° C. and stirred overnight. It was cooled to room temperature, filtered through celite, rinsed with methanol (150 mL) and the filtrate was concentrated under reduced pressure. The crude product was purified by reversed-phase flash chromatography (C₁₈) using 0 to 100% acetonitrile in water with 0.1% formic acid as modifier to afford 6-tert-butyl-2,5-dimethyl-pyridine-3-carbonitrile (841 mg, 71%). ESI-MS m/z calc. 188.13, found 189.2 (M+1)⁺. ¹H NMR (400 MHz, DMSO-d₆) δ (ppm) 7.93 (s, 1H), 2.58 (s, 3H), 2.47 (s, 3H), 1.37 (s, 9H).

### Step 6: 6-tert-butyl-2,5-dimethyl-pyridine-3-carboxylic acid

To a solution of 6-tert-butyl-2,5-dimethyl-pyridine-3-carbonitrile (840 mg, 4.46 mmol) in ethanol (10 mL) was added an aqueous solution of NaOH (5 mL of 10 M, 50 mmol). The reaction mixture was heated at 100° C. and stirred overnight. The reaction mixture was cooled to room temperature and ethanol was removed under reduced pressure. An aqueous solution of 6 M HCl was added (pH 7). The aqueous mixture was diluted with water (150 mL) and it was extracted with 2-MeTHF (10×150 mL). The combined organic layers were dried over sodium sulfate, filtered, and concentrated under reduced pressure. The crude product was purified by reversed-phase flash chromatography (C₁₈) using 5 to 45% acetonitrile in water with 0.1% formic acid to afford 6-tert-butyl-2,5-dimethyl-pyridine-3-carboxylic acid (851 mg, 92%) as a white solid. ESI-MS m/z calc. 207.13, found 208.2 (M+1)⁺. ¹H NMR (400 MHz, DMSO-d₆) δ (ppm) 12.97 (br s, 1H), 7.87 (s, 1H), 2.64 (s, 3H), 2.47 (s, 3H), 1.37 (s, 9H).

### Step 7: 5-bromo-2-tert-butyl-3,6-dimethyl-pyridine

A suspension of 6-tert-butyl-2,5-dimethyl-pyridine-3-carboxylic acid (5.62 g, 27.09 mmol), tetrabutylammonium tribromide (39.9 g, 82.75 mmol) and potassium phosphate (5.86 g, 27.61 mmol) in acetonitrile (120 mL) was purged with nitrogen for 20 minutes. The reaction mixture was heated at 100° C. and stirred for 48 h followed by stirring for 72 h at room temperature. The reaction mixture was filtered, rinsed with acetonitrile (200 mL) and concentrated under reduced pressure. The crude product was purified by silica gel flash chromatography using 0 to 5% ethyl acetate in heptanes to afford 5-bromo-2-tert-butyl-3,6-dimethyl-pyridine (3.91 g, 59%). ESI-MS m/z calc. 241.05, found 242.1 (M+1)⁺. ¹H NMR (400 MHz, CDCl₃) δ (ppm) 7.47 (s, 1H), 2.57 (s, 3H), 2.44 (s, 3H), 1.40 (s, 9H). ESI-MS m/z calc. 241.0466, found 242.1 (M+1)⁺.

### Step 8: 2-tert-butyl-3,6-dimethyl-5-(4,4,5,5-tetramethyl-1,3,2-dioxaborolan-2-yl)pyridine (Intermediate B-7)

2-tert-butyl-3,6-dimethyl-5-(4,4,5,5-tetramethyl-1,3,2-dioxaborolan-2-yl)pyridine (Intermediate B-7) was prepared from 5-bromo-2-tert-butyl-3,6-dimethyl-pyridine using a procedure analogous to that found in Intermediate B-1, Step 2. ¹H NMR (400 MHz, CDCl₃) δ (ppm) 7.68 (s, 1H), 2.66 (s, 3H), 2.45 (s, 3H), 1.41 (s, 9H), 1.34 (s, 12H).

### Intermediate B-8

### 2-(4-tert-butyl-2-ethyl-phenyl)-4,4,5,5-tetramethyl-1,3,2-dioxaborolane

### Step 1: 1-bromo-4-tert-butyl-2-ethyl-benzene

Under an inert atmosphere, acetic acid (10 mL) was placed in a 50 mL flask and 1-tert-butyl-3-ethyl-benzene (1 g, 6.16 mmol) was added followed by the addition of bromine (385 μL, 7.47 mmol). The reaction mixture was stirred at room temperature for 18 h The reaction mixture was poured onto ice and stirred for 20 minutes. Ethyl acetate was added and the layers were separated. The organic layer was washed with sodium bicarbonate (3×), followed by washing with water (2×). The organic layer was dried over magnesium sulfate, filtered, and concentrated. The crude material was purified via silica gel flash chromatography using 0 to 5% ethyl acetate in hexanes to obtain 1-bromo-4-tert-butyl-2-ethyl-benzene (1.2 g, 81%). ¹H NMR (400 MHz, CDCl₃) δ (ppm) 7.43 (d, J=8.4 Hz, 1H), 7.24 (d, J=2.5 Hz, 1H), 7.07 (dd, J=8.4, 2.5 Hz, 1H), 2.75 (q, J=7.5 Hz, 2H), 1.30 (s, 9H), 1.23 (t, J=7.5 Hz, 3H).

### Step 2: 2-(4-tert-butyl-2-ethyl-phenyl)-4,4,5,5-tetramethyl-1,3,2-dioxaborolane (Intermediate B-8)

2-(4-tert-butyl-2-ethyl-phenyl)-4,4,5,5-tetramethyl-1,3,2-dioxaborolane (Intermediate B-8, 852 mg, 59%) was prepared from 1-bromo-4-tert-butyl-2-ethyl-benzene using procedure analogous to that found in Intermediate B-1, Step 2. ESI-MS m/z calc. 288.23, found 289.26 (M+1)⁺. ¹H NMR (400 MHz, CDCl₃) δ (ppm) 7.75-7.71 (m, 1H), 7.21 (d, J=1.6 Hz, 2H), 2.91 (q, J=7.5 Hz, 2H), 1.32 (d, J=1.0 Hz, 12H), 1.31 (s, 9H), 1.20 (t, J=7.5 Hz, 3H).

### Intermediate B-9

### 2-[2,5-dimethyl-4-[1-(trifluoromethyl)vinyl]phenyl]-4,4,5,5-tetramethyl-1,3,2-dioxaborolane

### Step 1: 1-bromo-2,5-dimethyl-4-[1-(trifluoromethyl)vinyl]benzene

Under an atmosphere of nitrogen (4-bromo-2,5-dimethyl-phenyl)boronic acid (2.2 g, 9.61 mmol), 2-bromo-3,3,3-trifluoro-prop-1-ene (3. g, 17.15 mmol), PdCl₂(PPh₃)₂(340 mg, 0.48 mmol), potassium carbonate (5.3 g, 38.35 mmol) and THF (8 mL) were successively added to a microwave vial equipped with a magnetic stir bar. The reaction mixture was heated at 60° C. and stirred for 12 hours. The reaction mixture was cooled to room temperature, quenched with saturated ammonium chloride solution, and extracted with ether (3×40 mL). The combined organic phase was dried with anhydrous sodium sulfate and concentrated in vacuo. The crude material was purified via silica gel column chromatography using hexanes to obtain 1-bromo-2,5-dimethyl-4-[1-(trifluoromethyl)vinyl]benzene (1.52 g, 57%). ¹H NMR (400 MHz, DMSO-d₆) δ (ppm) 7.57 (s, 1H), 7.16 (s, 1H), 6.26 (q, J=1.6 Hz, 1H), 5.76 (q, J=1.3 Hz, 1H), 2.32 (s, 3H), 2.19 (s, 3H). ¹⁹F NMR (376 MHz, DMSO-d₆) δ (ppm) −65.58 (d, J=68.0 Hz).

### Step 2: 2-[2,5-dimethyl-4-[1-(trifluoromethyl)vinyl]phenyl]-4,4,5,5-tetramethyl-1,3,2-dioxaborolane (Intermediate B-9)

2-[2,5-dimethyl-4-[1-(trifluoromethyl)vinyl]phenyl]-4,4,5,5-tetramethyl-1,3,2-dioxaborolane (Intermediate B-9) (750 mg, 43%) was prepared from 1-bromo-2,5-dimethyl-4-[1-(trifluoromethyl)vinyl]benzene using procedure analogous to that found in Intermediate B-1, Step 2. ¹H NMR (400 MHz, CDCl₃) δ (ppm) 7.64 (d, J=2.3 Hz, 1H), 7.00 (s, 1H), 5.45 (d, J=1.4 Hz, 1H), 5.30 (s, 1H), 2.50 (d, J=2.3 Hz, 3H), 2.25 (d, J=2.3 Hz, 3H), 1.35 (d, J=7.2 Hz, 12H). ¹⁹F NMR (376 MHz, CDCl₃) δ (ppm) −66.92, −66.93.

### Intermediate B-10

### trimethyl-[3-methyl-4-(4,4,5,5-tetramethyl-1,3,2-dioxaborolan-2-yl)phenyl]silane

### Step 1: trimethyl-[3-methyl-4-(4,4,5,5-tetramethyl-1,3,2-dioxaborolan-2-yl)phenyl]silane (Intermediate B-10)

In a reaction vial, Pd(OAc)₂ (11 mg, 0.05 mmol), SPhos (52 mg, 0.13 mmol), bis(pinacolato)diboron (1.92 g, 7.56 mmol), and potassium phosphate (1.6 g, 7.54 mmol) were dissolved in dioxane (5 mL) and flushed with nitrogen (3×). (4-chloro-3-methyl-phenyl)-trimethyl-silane (500 mg, 2.52 mmol) was added and the reaction was stirred at room temperature for 48 h. The reaction was diluted with ethyl acetate and filtered through celite. The reaction mixture was then washed with saturated ammonium chloride solution, brine, dried over sodium sulfate, filtered and evaporated to dryness. The crude material was purified by silica gel column chromatography using 100% hexanes to obtain trimethyl-[3-methyl-4-(4,4,5,5-tetramethyl-1,3,2-dioxaborolan-2-yl)phenyl]silane (Intermediate B-10, 60.5 mg, 8%) as a white solid. ¹H NMR (400 MHz, CDCl₃) δ (ppm) 7.74 (d, J=7.6 Hz, 1H), 7.34-7.30 (m, 2H), 2.54 (s, 3H), 1.33 (s, 12H), 0.25 (s, 9H).

### Intermediate B-11

### 4,4,5,5-tetramethyl-2-[2-methyl-4-(1-methylcyclopropyl)phenyl]-1,3,2-dioxaborolane

### Step 1: 1-bromo-4-isopropenyl-2-methyl-benzene

In an oven-dried flask was added methyltriphenylphosphonium bromide (10.3 g, 28.83 mmol) in THF (38 mL). The reaction mixture was cooled to 0° C. and potassium tert-butoxide (3.2 g, 28.52 mmol) was added. The reaction mixture stirred for 45 minutes at 0° C. and a solution of 1-(4-bromo-3-methyl-phenyl)ethanone (5 g, 23.47 mmol) in THF (16 mL) was added dropwise. The resulting reaction mixture was gradually warmed to room temperature and stirred for 2 h. The solvent was evaporated, and the crude product was purified by silica gel column chromatography using 0 to 10% ethyl acetate in heptanes to afford 1-bromo-4-isopropenyl-2-methyl-benzene (4.35 g, 87%). ESI-MS m/z calc. 210 found 211.2 (M+1)⁺. GCMS m/z calc. 210.00, found 209.9 (M). ¹H NMR (400 MHz, CDCl₃) δ (ppm) 7.48 (d, J=8.3 Hz, 1H), 7.35-7.31 (m, 1H), 7.15 (dd, J=8.3, 2.0 Hz, 1H), 5.36 (s, 1H), 5.12-5.07 (m, 1H), 2.42 (s, 3H), 2.13 (s, 3H).

### Step 2: 1-bromo-2-methyl-4-(1-methylcyclopropyl)benzene

To DCM (50 mL) at 0° C. was added diethylzinc solution in hexanes (50 mL of 1 M, 50 mmol) followed by a dropwise addition of trifluoroacetic acid (6.66 g, 4.5 mL, 58.41 mmol) solution in DCM (13 mL). After stirring for 15 minutes, diiodomethane (15.3 g, 4.6 mL, 57.11 mmol) as a solution in DCM (13 mL) was added and the reaction mixture was stirred for 15 minutes. 1-Bromo-4-isopropenyl-2-methyl-benzene (4.35 g, 20.61 mmol) in DCM (23 mL) was then added and the mixture was stirred for 3 hours at room temperature. The reaction mixture was quenched with a 1 M hydrochloric acid solution (50 mL) and the layers were separated. The aqueous layer was extracted with DCM (2×100 mL). The combined organic layer was washed with saturated sodium bicarbonate (100 mL), sodium sulfite (100 mL) and brine (100 mL). The organic phase was dried over anhydrous sodium sulfate, filtered and concentrated in vacuo. The crude product was purified via silica gel column chromatography using 100% heptanes to afford 1-bromo-2-methyl-4-(1-methylcyclopropyl)benzene (6.25 g, 74%) as a light yellow oil. ¹H NMR (400 MHz, CDCl₃) δ (ppm) 7.42 (d, J=8.1 Hz, 1H), 7.13 (d, J=2.0 Hz, 1H), 6.94 (dd, J=8.3, 2.0 Hz, 1H), 2.38 (s, 3H), 1.38 (s, 3H), 0.85-0.80 (m, 2H), 0.75-0.70 (m, 2H).

### Step 3: 4,4,5,5-tetramethyl-2-[2-methyl-4-(1-methylcyclopropyl)phenyl]-1,3,2-dioxaborolane (Intermediate B-11)

4,4,5,5-tetramethyl-2-[2-methyl-4-(1-methylcyclopropyl)phenyl]-1,3,2-dioxaborolane (Intermediate B-11) was prepared from 1-bromo-2-methyl-4-(1-methylcyclopropyl)benzene using procedure analogous to that found in Intermediate B-1, Step 2. ESI-MS m/z calc. 272.19, found 273.2 (M+1)⁺. ¹H NMR (400 MHz, DMSO-d₆) δ (ppm) 7.53 (d, J=7.6 Hz, 1H), 7.02-6.96 (m, 2H), 2.43 (s, 3H), 1.35 (s, 3H), 1.28 (s, 12H), 0.85-0.79 (m, 2H), 0.78-0.73 (m, 2H).

### Intermediate B-12

### 2-tert-butyl-3-chloro-6-methyl-5-(4,4,5,5-tetramethyl-1,3,2-dioxaborolan-2-yl)pyridine

### Step 1: 6-tert-butyl-5-chloro-2-hydroxy-pyridine-3-carbonitrile

A solution of 6-tert-butyl-2-hydroxy-pyridine-3-carbonitrile (5 g, 28.35 mmol) and NCS (4.7 g, 35.2 mmol) in anhydrous 1,2-dichloroethane (25 mL) was stirred at 80° C. for 4 h Once cooled to room temperature, the reaction mixture was treated with saturated aqueous sodium bicarbonate solution (100 mL) It was diluted with water (100 mL) and the aqueous layer was extracted with DCM (2×300 mL). The combined organic layer was washed with water (200 mL), brine (200 mL), dried over sodium sulfate, filtered and concentrated under reduced pressure to give 6-tert-butyl-5-chloro-2-hydroxy-pyridine-3-carbonitrile (5.99 g, 100%) as a beige solid. ESI-MS m/z calc. 210.06, found 211.2 (M+1)⁺. ¹H NMR (400 MHz, CDCl₃) δ (ppm) 9.94 (br s, 1H), 7.78 (s, 1H), 1.53 (s, 9H).

### Step 2: 2-bromo-6-tert-butyl-5-chloro-pyridine-3-carbonitrile

To a stirred suspension of 6-tert-butyl-5-chloro-2-hydroxy-pyridine-3-carbonitrile (5.9 g, 27.98 mmol) in toluene (90 mL) was added phosphorus oxybromide (11 g, 38.37 mmol). The reaction mixture was stirred at 95° C. for 16 h After the reaction mixture was cooled to room temperature, it was quenched by slow addition of saturated aqueous sodium bicarbonate solution (150 mL). The mixture was poured in a separatory funnel and diluted with water (200 mL). The aqueous layer was extracted with ethyl acetate (2×200 mL). The combined organic layer was washed with brine (100 mL), dried over sodium sulfate, filtered and concentrated under reduced pressure. The crude product was purified via silica gel column chromatography using 0 to 10% ethyl acetate in heptane to give 2-bromo-6-tert-butyl-5-chloro-pyridine-3-carbonitrile (6.29 g, 82%) as a dark orange oil. ESI-MS m/z calc. 271.97, found 273.0 (M+2)⁺. ¹H NMR (400 MHz, CDCl₃) δ (ppm) 7.81 (s, 1H), 1.48 (s, 9H).

### Step 3: 6-tert-butyl-5-chloro-2-methyl-pyridine-3-carboxylic acid

To a solution of 6-tert-butyl-5-chloro-2-methyl-pyridine-3-carbonitrile (2.09 g, 10 mmol) in ethanol (32 mL) was added aqueous NaOH (15 mL of 10 M, 150 mmol). The pale-yellow solution was stirred at 100° C. in a sealed tube for 24 h. The solvent was removed under reduced pressure and the aqueous residue was diluted with water (100 mL). An insoluble white solid precipitated formed which was removed by filtration and rinsed with water (50 mL). The filtrate was washed with MTBE (2×50 mL). The pH was adjusted to ˜4 by addition of aqueous 3M HCl (˜15 mL) and aqueous layer was extracted with DCM (3×100 mL). The combined organic layer was dried over anhydrous sodium sulfate, filtered and concentrated under reduced pressure to give crude 6-tert-butyl-5-chloro-2-methyl-pyridine-3-carboxylic acid (2.13 g, 93%) as a white solid. ESI-MS m/z calc. 227.07, found 228.2 (M+1)⁺. ¹H NMR (400 MHz, CDCl₃) δ (ppm) 8.23 (s, 1H), 2.82 (s, 3H), 1.50 (s, 9H).

### Step 4: 5-bromo-2-tert-butyl-3-chloro-6-methyl-pyridine

A flame-dried round-bottom flask was charged with 6-tert-butyl-5-chloro-2-methyl-pyridine-3-carboxylic acid (2.02 g, 8.86 mmol), potassium phosphate tribasic (3.7 g, 17.43 mmol), tetrabutylammonium tribromide (19 g, 39.41 mmol) and anhydrous acetonitrile (40 mL). The resulting reaction mixture was refluxed at 100° C. for 95 h. The reaction mixture was cooled to room temperature and was concentrated under reduced pressure. The residue was purified by silica gel column chromatography using 0 to 15% ethyl acetate in heptanes to give 5-bromo-2-tert-butyl-3-chloro-6-methyl-pyridine (1.24 g, 53%) as a colorless oil. ¹H NMR (400 MHz, CDCl₃) δ (ppm) 7.72 (s, 1H), 2.58 (s, 3H), 1.45 (s, 9H).

### Step 5: 2-tert-butyl-3-chloro-6-methyl-5-(4,4,5,5-tetramethyl-1,3,2-dioxaborolan-2-yl)pyridine (Intermediate B-12)

2-tert-butyl-3-chloro-6-methyl-5-(4,4,5,5-tetramethyl-1,3,2-dioxaborolan-2-yl)pyridine (Intermediate B-12) was prepared from 5-bromo-2-tert-butyl-3-chloro-6-methyl-pyridine using procedure analogous to that found in Intermediate B-1, Step 2 as a white solid. ESI-MS m/z calc. 309.17, found 310.1 (M+1)⁺. ¹H NMR (400 MHz, CDCl₃) δ (ppm) 7.89 (s, 1H), 2.65 (s, 3H), 1.47 (s, 9H), 1.33 (s, 12H).

### Intermediate B-13

### 2-[4-tert-butyl-2-methyl-3-(trifluoromethyl)phenyl]-4,4,5,5-tetramethyl-1,3,2-dioxaborolane (Intermediate B-13A) and 2-[4-tert-butyl-2-methyl-5-(trifluoromethyl)phenyl]-4,4,5,5-tetramethyl-1,3,2-dioxaborolane (Intermediate B-13B)

### Step 1: 1-bromo-4-tert-butyl-2-methyl-benzene

Under an inert atmosphere, acetic acid (40 mL) was placed in a 50 mL flask and 1-tert-butyl-3-methyl-benzene (3.67 g, 24.76 mmol) was added, followed by the addition of Bromine (1.55 mL, 30.09 mmol). The reaction mixture was stirred for 18 h at room temperature and poured onto ice and stirred for 20 minutes. Ethyl acetate was added, and the layers were separated. The organic layer was washed with sodium thiosulphate solution, followed by washing with saturated sodium bicarbonate solution and water (2×). The organic layer was dried over magnesium sulfate, filtered, and concentrated. The crude material was purified via silica gel column chromatography using hexanes to obtain 1-bromo-4-tert-butyl-2-methyl-benzene (4.6 g, 67%) ¹H NMR (400 MHz, CDCl₃) δ (ppm) 7.43 (d, J=8.4 Hz, 1H), 7.24-7.22 (m, 1H), 7.06 (dd, J=8.4, 2.5 Hz, 1H), 2.39 (s, 3H), 1.29 (s, 9H).

### Step 2: 1-bromo-4-tert-butyl-2-methyl-3-(trifluoromethyl)benzene;1-bromo-4-tert-butyl-2-methyl-5-(trifluoromethyl)benzene; and 2-bromo-5-tert-butyl-1-methyl-3-(trifluoromethyl)benzene

A solution of 1-bromo-4-tert-butyl-2-methyl-benzene (1,400 mg, 6.16 mmol), tetramethylguanidine-trifluoromethyl iodide adduct (2 g, 6.43 mmol), copper diacetate hydrate (2.5 g, 12.52 mmol) and potassium peroxodisulfate (6.66 g, 24.66 mmol) in acetic acid (40 mL) was stirred at 90° C. overnight. The reaction mixture was quenched with ethyl acetate, filtered, and concentrated in vacuo. The obtained residue was purified by silica gel column chromatography using hexanes to give a mixture of 1-bromo-4-tert-butyl-2-methyl-3-(trifluoromethyl)benzene, 1-bromo-4-tert-butyl-2-methyl-5-(trifluoromethyl)benzene and 2-bromo-5-tert-butyl-1-methyl-3-(trifluoromethyl)benzene (1,000 mg, 55%). The mixture was used as is in the next step.

### Step 3: 2-[4-tert-butyl-2-methyl-3-(trifluoromethyl)phenyl]-4,4,5,5-tetramethyl-1,3,2-dioxaborolane (Intermediate B-13A) and 2-[4-tert-butyl-2-methyl-5-(trifluoromethyl)phenyl]-4,4,5,5-tetramethyl-1,3,2-dioxaborolane (Intermediate B-13B)

To a microwave reaction vial containing a mixture of 1-bromo-4-tert-butyl-2-methyl-3-(trifluoromethyl)benzene, 1-bromo-4-tert-butyl-2-methyl-5-(trifluoromethyl)benzene and 2-bromo-5-tert-butyl-1-methyl-3-(trifluoromethyl)benzene (1.320 g, 4.47 mmol) was added triethylamine (1.36 g, 13.44 mmol) and Pd(dppf)₂Cl₂·DCM (73.06 mg, 0.09 mmol) in dioxane (12 mL). The reaction mixture was purged with nitrogen and pinacolborane (1.8 g, 14.07 mmol) was added. The vial was sealed, and the reaction mixture was subjected to microwave irradiation at 140° C. for 1 h. The reaction mixture was then quenched with water and extracted with ethyl acetate. The organic layer was separated, washed with brine, dried over sodium sulphate, filtered, and concentrated. The crude material was purified by preparative reverse phase HPLC (C₁₈) using 1 to 99% ACN in water (HCl modifier) to give ˜2:1 a mixture 2-[4-tert-butyl-2-methyl-3-(trifluoromethyl)phenyl]-4,4,5,5-tetramethyl-1,3,2-dioxaborolane (Intermediate B-13A) and 2-[4-tert-butyl-2-methyl-5-(trifluoromethyl)phenyl]-4,4,5,5-tetramethyl-1,3,2-dioxaborolane (Intermediate B-13B) (27.8 mg, 2%). ESI-MS m/z calc. 342.19, found 343.3 (M+1)⁺.

Alternatively, Intermediate B-13B can be prepared via the following method:

### Step 1: (5-benzyloxy-2-tert-butyl-4-methyl-phenyl)boronic acid

A tube was charged with 1-benzyloxy-5-bromo-4-tert-butyl-2-methyl-benzene (100 mg, 0.27 mmol), XPhos Pd G4 (20 mg, 0.02 mmol), XPhos (22 mg, 0.05 mmol), hypoboric acid (102 mg, 1.14 mmol), potassium acetate (115 mg, 1.17 mmol) and ethanol (2 mL). The solution was bubbled with nitrogen for 5 minutes, sealed and stirred at 85° C. for 18 h. The crude was partitioned between water (20 mL) and ethyl acetate (20 mL). The aqueous phase was extracted with ethyl acetate (2×20 mL). The combined organic layer was dried over anhydrous sodium sulfate, filtered and concentrated under reduced pressure. The crude product was purified by reversed-phase chromatography (C₁₈) using 2 to 60% acetonitrile in water with 0.1% formic acid). to afford (5-benzyloxy-2-tert-butyl-4-methyl-phenyl)boronic acid (62 mg, 74%) as a white solid. ¹H NMR (400 MHz, CD₃OD) δ (ppm) 7.51-7.43 (m, 2H), 7.42-7.35 (m, 2H), 7.34-7.27 (m, 1H), 7.21 (s, 1H), 6.84 (s, 1H), 5.09 (s, 2H), 2.23 (s, 3H), 1.39 (s, 9H).

### Step 2: 1-benzyloxy-4-tert-butyl-2-methyl-5-(trifluoromethyl)benzene

To a solution of (5-benzyloxy-2-tert-butyl-4-methyl-phenyl)boronic acid (1.58 g, 5.29 mmol), copper(I) chloride (580 mg, 5.86 mmol) and sodium trifluoromethanesulfinate (2.7 g, 17.3 mmol) in a mixture of methanol (21 mL), DCM (21 mL) and water (17 mL) at room temperature was slowly added tert-butyl hydroperoxide in water (4.3 mL of 70% w/v, 33.4 mmol) and the reaction was stirred at room temperature for 18 h. Additional tert-butyl hydroperoxide in water (2 mL of 70% w/v, 15.54 mmol), copper(I) chloride (270 mg, 2.73 mmol) and sodium trifluoromethanesulfinate (1.7 g, 10.89 mmol) were added and the reaction was stirred for an additional 18 h. The reaction mixture was diluted with water (50 mL) and extracted with DCM (2×50 mL). The combined organic layer was dried over sodium sulfate, filtered and concentrated under reduced pressure. The crude product was purified by silica gel chromatography using heptanes to afford 1-benzyloxy-4-tert-butyl-2-methyl-5-(trifluoromethyl)benzene (1.01 g, 58%) as a colorless oil. ¹H NMR (400 MHz, CDCl₃) δ (ppm) 7.51-7.31 (m, 6H), 7.23 (s, 1H), 5.09 (s, 2H), 2.30 (s, 3H), 1.45 (s, 9H). ¹⁹F NMR (377 MHz, CDCl₃) δ (ppm) −52.61 (s, 3F).

### Step 3: 4-tert-butyl-2-methyl-5-(trifluoromethyl)phenol

A solution of 1-benzyloxy-4-tert-butyl-2-methyl-5-(trifluoromethyl)benzene (210 mg, 0.65 mmol) in methanol (5 mL) was treated with Pd/C (69 mg of 10% w/w, 0.065 mmol) and sparged with hydrogen from the balloon for 1 h. The reaction mixture was filtered and concentrated to give 4-tert-butyl-2-methyl-5-(trifluoromethyl)phenol (150 mg, 99%). ¹H-NMR (400 MHz, CDCl₃) δ (ppm) 7.36 (s, 1H), 7.12 (s, 1H), 4.87 (s, 1H), 2.27 (s, 3H), 1.41 (s, 9H). ¹⁹F NMR (376 MHz, CDCl₃) δ (ppm) −52.94.

### Step 4: [4-tert-butyl-2-methyl-5-(trifluoromethyl)phenyl] trifluoromethanesulfonate

A mixture of 4-tert-butyl-2-methyl-5-(trifluoromethyl)phenol (500 mg, 2.15 mmol) and pyridine (530 μL, 6.55 mmol) in DCM (15 mL) was carefully treated with trifluoromethylsulfonyl trifluoromethanesulfonate (550 μL, 3.27 mmol) at 0° C. The mixture was stirred overnight at room temperature and quenched with water and DCM. Organic layer was separated, dried over sodium sulfate, filtered and evaporated in vacuo. The crude material was purified by silica gel chromatography using 0 to 5% ethyl acetate in hexanes to give [4-tert-butyl-2-methyl-5-(trifluoromethyl)phenyl]trifluoromethanesulfonate (732 mg, 93%). ¹H NMR (400 MHz, CDCl₃) δ (ppm) 7.57 (s, 1H), 7.55 (s, 1H), 2.42 (s, 3H), 1.45 (s, 9H). ¹⁹F NMR (376 MHz, CDCl₃) δ (ppm) −53.38, −73.62.

### Step 5: 2-[4-tert-butyl-2-methyl-5-(trifluoromethyl)phenyl]-4,4,5,5-tetramethyl-1,3,2-dioxaborolane (Intermediate B-13B)

A microwave reaction vial charged with [4-tert-butyl-2-methyl-5-(trifluoromethyl)phenyl]trifluoromethanesulfonate (730 mg, 2.004 mmol), Pd(dppf)Cl₂·DCM (82 mg, 0.1 mmol), Et₃N (850 μL, 6.1 mmol) and dioxane (10 mL) was purged with nitrogen and pinacolborane (870 μL, 6 mmol) was added under nitrogen atmosphere and the vial was sealed. The reaction mixture was subjected to microwave irradiation at 140° C. for 1 h. The mixture was quenched with water and extracted with ethyl acetate. The organic layer was washed with brine, dried over sodium sulfate, filtered and concentrated. The residue was purified by silica gel column chromatography using hexanes to give 2-[4-tert-butyl-2-methyl-5-(trifluoromethyl)phenyl]-4,4,5,5-tetramethyl-1,3,2-dioxaborolane (Intermediate B-13B, 500 mg, 69%) as a yellow oil. ESI-MS m/z calc. 342.19, found 343.3 (M+1)⁺. ¹H NMR (400 MHz, CDCl₃) δ (ppm) 8.09 (s, 1H), 7.41 (s, 1H), 2.56 (s, 3H), 1.43 (s, 9H), 1.33 (s, 12H). ¹⁹F NMR (376 MHz, CDCl₃) δ (ppm) −52.36.

### Intermediate B-14

### 2,5-dimethyl-3-(4,4,5,5-tetramethyl-1,3,2-dioxaborolan-2-yl)-6-(trifluoromethyl)pyridine

### Step 1: 4-ethoxy-1,1,1-trifluoro-3-methyl-but-3-en-2-one

(2,2,2-Trifluoroacetyl) 2,2,2-trifluoroacetate (19.04 g, 12.6 mL, 90.65 mmol) was added to a solution of 1-ethoxyprop-1-ene (7.7800 g, 10 mL, 90.37 mmol) and pyridine (7.14 g, 7.3 mL, 90.26 mmol) in DCM (160 mL) at 0° C. The reaction mixture was stirred at room temperature for 18 h. The reaction mixture was diluted with DCM (150 mL). The organic layer was washed with water (100 mL), 1 M aqueous HCl (100 mL) and brine (100 mL), dried over sodium sulfate, filtered, and concentrated under reduced pressure to give 4-ethoxy-1,1,1-trifluoro-3-methyl-but-3-en-2-one (17.3 g, 96%) as a yellow oil. ¹H NMR (CDCl₃, 300 MHz) δ (ppm) 7.55-7.51 (m, 1H), 4.21 (q, J=7.1 Hz, 2H), 1.81-1.78 (m, 3H), 1.40 (t, J=7.1 Hz, 3H). ¹⁹F NMR (CDCl₃, 282 MHz) δ (ppm) −69.25 (s, 3F).

### Step 2: methyl 2-hydroxy-5-methyl-6-(trifluoromethyl)pyridine-3-carboxylate

To a solution of 4-ethoxy-1,1,1-trifluoro-3-methyl-but-3-en-2-one (14.02 g, 76.97 mmol) and ethyl 3-amino-3-oxo-propanoate (8.2 g, 70.024 mmol) in methanol (305 mL) was added sodium methoxide (solid) (4.92 g, 91.07 mmol). The reaction mixture was heated at reflux for 3 h, cooled to room temperature and stirred overnight. The precipitate was removed by filtration and washed with methanol (400 mL) and water (400 mL). The resultant solid was partitioned between 2N hydrochloric acid (300 mL) and ethyl acetate (300 mL). The remaining solid was removed by filtration and the aqueous phase extracted further with ethyl acetate (2×250 mL). The combined organic layer was washed with water (300 mL) and brine (300 mL), dried over sodium sulfate and concentrated in vacuo to give cream solid methyl 2-hydroxy-5-methyl-6-(trifluoromethyl)pyridine-3-carboxylate (10.94 g, 65%). ESI-MS m/z calc. 235.05, found 236.08 (M+1)⁺. ¹H-NMR (400 MHz, CDCl₃) δ (ppm) 8.15 (s, 1H), 4.01 (s, 3H), 2.43 (q, J=2.0 Hz, 3H).

### Step 3: methyl 2-chloro-5-methyl-6-(trifluoromethyl)pyridine-3-carboxylate

A mixture of dichlorophosphoryloxybenzene (46.6 g, 33 mL, 220.85 mmol) and methyl 2-hydroxy-5-methyl-6-(trifluoromethyl)pyridine-3-carboxylate (8.8 g, 37.42 mmol) was heated to 160° C. for 6 h. The cooled reaction mixture was slowly poured onto a stirring mixture of ethyl acetate (200 mL) and saturated aqueous sodium bicarbonate (200 mL). Halfway through the addition, a few ice cubes were added to cool the slightly exothermic reaction. The mixture was stirred vigorously for 30 minutes. The layers were separated (pH 1-2). The organic layer was washed with brine (50 mL), dried over sodium sulfate, filtered, and concentrated under reduced pressure. The crude mixture was poured onto a stirring mixture of ethyl acetate (250 mL), water (250 mL) and sodium carbonate (24 g) and was stirred vigorously for 1 h. The layers were separated (pH 6-7). The organic layer was washed with brine (50 mL), dried over sodium sulfate, filtered and concentrated under reduced pressure to give crude methyl 2-chloro-5-methyl-6-(trifluoromethyl)pyridine-3-carboxylate (8 g, 70%) as a white solid. ¹H NMR (300 MHz, CDCl₃) δ (ppm) 8.10 (s, 1H), 3.98 (s, 3H), 2.54-2.48 (m, 3H).

### Step 4: methyl 2,5-dimethyl-6-(trifluoromethyl)pyridine-3-carboxylate

A mixture of methyl 2-chloro-5-methyl-6-(trifluoromethyl)pyridine-3-carboxylate (1 g, 3.68 mmol), trimethylaluminium in toluene (2 mL of 2 M, 4 mmol), and Pd(PPh₃)₄(213 mg, 0.18 mmol) in dioxane (8 mL) was stirred at 90° C. under argon for 90 minutes. The reaction mixture was cooled to room temperature and poured onto 1M aqueous HCl (50 mL), extracted with ethyl acetate (2×50 mL). The combined organic extracts were washed with brine (50 mL), dried over magnesium sulfate and concentrated. Purification by silica gel column chromatography using 0 to 5% ethyl acetate in heptane gave methyl 2,5-dimethyl-6-(trifluoromethyl)pyridine-3-carboxylate (547 mg, 62%) as a colorless oil. ESI-MS m/z calc. 233.06, found 233.98 (M+1)⁺. ¹H-NMR (400 MHz, CDCl₃) δ (ppm) 8.12 (s, 1H), 3.95 (s, 3H), 2.83 (s, 3H), 2.50 (d, J=1.1 Hz, 3H). ¹⁹F-NMR (376 MHz, CDCl₃) δ (ppm) −65.5 (d, J=1.6 Hz, 3F).

### Step 5: 2,5-dimethyl-6-(trifluoromethyl)pyridine-3-carboxylic acid

To a solution of methyl 2,5-dimethyl-6-(trifluoromethyl)pyridine-3-carboxylate (540 mg, 2.26 mol) in THF (5 mL), methanol (2.5 mL) and water (2.5 mL) was added lithium hydroxide monohydrate (300 mg, 7.15 mmol). The resulting mixture was stirred at room temperature for 1 h. The reaction was concentrated, and the residue was dissolved in water (10 mL). The pH was adjusted to ˜pH 3 with 2M HCl and the aqueous layer was extracted with ethyl acetate (2×20 mL). The combined organic layer was washed with brine (20 mL), dried over magnesium sulfate, filtered and concentrated to give 2,5-dimethyl-6-(trifluoromethyl)pyridine-3-carboxylic acid (367 mg, 73%) as a white solid. ESI-MS m/z calc. 219.05, found 219.98 (M+1)⁺. ¹H-NMR (400 MHz, CDCl₃) δ (ppm) 8.27 (s, 1H), 2.90 (s, 3H), 2.53 (d, J=2.3 Hz, 3H). ¹⁹F-NMR (376 MHz, CDCl₃) δ (ppm) −65.6 (d, J=1.3 Hz, 3F).

### Step 6: tert-butyl N-[2,5-dimethyl-6-(trifluoromethyl)-3-pyridyl]carbamate

To a solution of 2,5-dimethyl-6-(trifluoromethyl)pyridine-3-carboxylic acid (250 mg, 1.12 mmol) in toluene (2.5 mL) was added triethylamine (181.50 mg, 0.25 mL, 1.79 mmol) and diphenylphosphoryl azide (383.10 mg, 0.3 mL, 1.39 mmol). The mixture was stirred at room temperature for 30 minutes and tert-butanol (1 mL) was added. The reaction mixture was heated at 110° C. for 2 h, cooled to room temperature and diluted with ethyl acetate (20 mL) and water (20 mL). The organic layer was separated, washed with brine (20 mL), dried over magnesium sulfate and concentrated to give tert-butyl N-[2,5-dimethyl-6-(trifluoromethyl)-3-pyridyl]carbamate (295 mg, 67%) as an off-white solid. ESI-MS m/z calc. 290.12, found 291.03 (M+1)⁺. ¹H-NMR (400 MHz, CDCl₃) δ (ppm) 8.29 (s, 1H), 6.42 (s, 1H), 2.50 (s, 3H), 2.45 (s, 3H), 1.55 (s, 9H). ¹⁹F-NMR (376 MHz, CDCl₃) δ (ppm) −64.1 (s, 3F).

### Step 7: 2,5-dimethyl-6-(trifluoromethyl)pyridin-3-amine

A solution of tert-butyl N-[2,5-dimethyl-6-(trifluoromethyl)-3-pyridyl]carbamate (3.05 g, 9.75 mol) in HCl in dioxane (20 mL of 4 M, 80 mmol) was stirred at room temperature for 16 h. Concentrated to give 2,5-dimethyl-6-(trifluoromethyl)pyridin-3-amine (Hydrochloride Salt (2)) (2.58 g, 97%) as a white solid. ESI-MS m/z calc. 190.07, found 190.99 (M+1)⁺. ¹H-NMR (400 MHz, CD₃OD) δ (ppm) 7.42 (s, 1H), 2.57 (s, 3H), 2.47 (d, J=1.8 Hz, 3H). ¹⁹F-NMR (376 MHz, CD₃OD) δ (ppm) −63.0 (s, 3F).

### Step 8: 3-bromo-2,5-dimethyl-6-(trifluoromethyl)pyridine

To a stirred mixture of 2,5-dimethyl-6-(trifluoromethyl)pyridin-3-amine (1.49 g, 7.60 mmol) and copper (II) bromide (3.74 g, 16.74 mmol) in anhydrous acetonitrile (38 mL) at −10° C. was added tert-butyl nitrite (1.99 g, 2.3 mL, 19.34 mmol) dropwise. The mixture was warmed to room temperature over 2 h and diluted with water (40 mL). The aqueous layer was extracted with DCM (2×50 mL). The combined organic layer was washed with brine, dried over anhydrous sodium sulfate and concentrated. The crude was purified by flash silica gel column chromatography using 0 to 5% ethyl acetate in heptanes to give 3-bromo-2,5-dimethyl-6-(trifluoromethyl)pyridine (1.47 g, 75%). ¹H-NMR (400 MHz, CDCl₃) δ (ppm) 7.77 (s, 1H), 2.66 (s, 3H), 2.43 (d, J=2.3 Hz, 3H).

### Step 9: 2,5-dimethyl-3-(4,4,5,5-tetramethyl-1,3,2-dioxaborolan-2-yl)-6-(trifluoromethyl)pyridine (Intermediate B-14)

To a solution of 3-bromo-2,5-dimethyl-6-(trifluoromethyl)pyridine (1.46 g, 5.64 mmol) in toluene (24 mL) at −78° C. was added n-BuLi in hexanes (4.2 mL of 1.6 M, 6.72 mmol) dropwise and the mixture was stirred at this temperature for 20 minutes then at 0° C. for 30 minutes. The mixture was re-cooled to −78° C. and a solution of 2-isopropoxy-4,4,5,5-tetramethyl-1,3,2-dioxaborolane (1.68 g, 9.03 mmol) in toluene (24 mL) was added dropwise. After 15 minutes the solution was warmed to 0° C. and stirred at this temperature for 1 h. The reaction was quenched by the addition of saturated ammonium chloride solution (25 mL). The reaction mixture was diluted with water (25 mL) and extracted with ethyl acetate (2×50 mL). The combined organic layer was washed with water (40 mL), brine (40 mL) and dried over sodium sulfate. Purification by silica gel column chromatography using 2 to 80% ethyl acetate in heptanes followed by second purification via reverse phase chromatography (C₁₈) using 0 to 30% ACN in water containing 0.1% NH3 gave 2,5-dimethyl-3-(4,4,5,5-tetramethyl-1,3,2-dioxaborolan-2-yl)-6-(trifluoromethyl)pyridine (Intermediate B-14, 388 mg, 22%). 301.15, found 302.07 (M+1)⁺. ¹H-NMR (400 MHz, CDCl₃) δ (ppm) 7.97 (s, 1H), 2.76 (s, 3H), 2.45 (s, 3H), 1.38 (s, 12H)

### Intermediate B-15

### 1,5-dimethyl-6-(4,4,5,5-tetramethyl-1,3,2-dioxaborolan-2-yl)indole

### Step 1: 6-bromo-1,5-dimethyl-indole

Sodium hydride (500 mg of 60% w/w, 12.50 mmol) was added to a solution of 6-bromo-5-methyl-1H-indole (1.150 g, 5.47 mmol) in THF (20 mL) at 0° C. The reaction mixture was stirred at this temperature for 30 minutes. Iodomethane (1.08 g, 7.61 mmol) was added dropwise, and the reaction mixture was stirred at room temperature for 16 h. The reaction mixture was diluted with water (20 ml) and extracted with ethyl acetate (3×). The combined organic layer was washed with brine, dried over sodium sulfate and concentrated under reduced pressure. Purification by silica gel column chromatography using 0 to 10% ethyl acetate in hexane gave 6-bromo-1,5-dimethyl-indole (1.1824 g, 77%) ¹H NMR (400 MHz, DMSO-d₆) δ (ppm) 7.72 (s, 1H), 7.51 (s, 1H), 7.30 (d, J=3.0 Hz, 1H), 6.35 (d, J=3.1 Hz, 1H), 3.76 (s, 3H), 2.40 (s, 3H).

### Step 2: 1,5-dimethyl-6-(4,4,5,5-tetramethyl-1,3,2-dioxaborolan-2-yl)indole (Intermediate B-15)

1,5-dimethyl-6-(4,4,5,5-tetramethyl-1,3,2-dioxaborolan-2-yl)indole (Intermediate B-15) was prepared from 6-bromo-1,5-dimethyl-indole using procedure analogous to that found in Intermediate B-1, Step 2. ESI-MS m/z calc. 271.17, found 272.0 (M+1)⁺. ¹H NMR (400 MHz, CD₃OD) δ (ppm) 7.76 (s, 1H), 7.29 (s, 1H), 7.14 (d, J=3.1 Hz, 1H), 6.30 (d, J=3.0 Hz, 1H), 3.78 (s, 3H), 2.56 (s, 3H), 1.37 (s, 12H).

### Intermediate B-16

### 4,4,5,5-tetramethyl-2-(1,1,4,7-tetramethylindan-5-yl)-1,3,2-dioxaborolane

### Step 1: 5-bromo-4,7-dimethyl-indan-1-one

To a flask charged with aluminium chloride (900 μL, 16.47 mmol) was added degassed DCM (30 mL) under an atmosphere of nitrogen. 3-chloropropanoyl chloride (2.5 g, 19.69 mmol) was added slowly and the reaction mixture was stirred for 15 minutes to obtain a reddish-brown solution. A solution of 2-bromo-1,4-dimethyl-benzene (3 g, 16.21 mmol) in DCM (2 mL) was added slowly and the reaction mixture was stirred for 20 h at room temperature (no HCl evolution observed). The reaction mixture was poured onto ice and extracted with DCM (3×). The combined organic phase was washed with saturated sodium bicarbonate solution and dried over magnesium sulfate, filtered and concentrated to obtain 1-(4-bromo-2,5-dimethyl-phenyl)-3-chloro-propan-1-one (4.1 g, 92%) as a white solid. ¹H NMR (400 MHz, CDCl₃) δ (ppm) 7.50 (s, 1H), 7.46 (s, 1H), 3.89 (t, J=6.6 Hz, 2H), 3.35 (t, J=6.6 Hz, 2H), 2.46 (s, 3H), 2.42 (s, 3H).

To a 100 mL RBF equipped with a condenser, the above crude material and sulfuric acid (40 mL, 750.4 mmol) were added. The reaction mixture was heated at 100° C. for 4 h, under an atmosphere of nitrogen. The reaction mixture was cooled to room temperature and poured onto ice. The product crashed out, and was filtered and washed with water. The crude material was taken up in DCM, dried over magnesium sulfate, filtered and concentrated to obtain 5-bromo-4,7-dimethyl-indan-1-one (1.98 g, 51%), as a white solid. ¹H NMR (400 MHz, CDCl₃) δ (ppm) 7.34 (s, 1H), 3.00 (t, 2H), 2.68 (t, 2H), 2.57 (s, 3H), 2.36 (s, 3H).

### Step 2: 5-bromo-1,1,4,7-tetramethyl-indane

To a solution of tetrachlorotitanium in toluene (17 mL of 1 M, 17.00 mmol) and DCM (12 mL) at −40° C. was added dimethylzinc in toluene (12 mL of 2 M, 24 mmol) slowly maintaining the internal temperature below −40° C. The reaction mixture was stirred at −40° C. for 30 minutes and then a solution of 5-bromo-4,7-dimethyl-indan-1-one (1.98 g, 8.28 mmol) in DCM (3 mL) was added dropwise, maintaining the internal temperature below −40° C. The reaction mixture was gradually warmed to room temperature and stirred for 16 h. The reaction mixture was quenched by slowly pouring it into an ice/saturated sodium bicarbonate solution. The aqueous phase was acidified with concentrated HCl and extracted with DCM (3×). The organic layer was combined, dried over magnesium sulfate, filtered, and concentrated in vacuo. The crude material was purified via silica gel column chromatography using hexanes to obtain 5-bromo-1,1,4,7-tetramethyl-indane (1.77 g, 84%). ¹H NMR (400 MHz, DMSO-d₆) δ (ppm) 7.14 (s, 1H), 2.78 (t, J=7.3 Hz, 2H), 2.28 (s, 3H), 2.21 (s, 3H), 1.85 (t, J=7.3 Hz, 2H), 1.28 (s, 6H).

### Step 3: 4,4,5,5-tetramethyl-2-(1,1,4,7-tetramethylindan-5-yl)-1,3,2-dioxaborolane (Intermediate B-16)

4,4,5,5-tetramethyl-2-(1,1,4,7-tetramethylindan-5-yl)-1,3,2-dioxaborolane (Intermediate B-16) was prepared from 5-bromo-1,1,4,7-tetramethyl-indane using procedure analogous to that found in Intermediate B-1, Step 2. ¹H NMR (400 MHz, DMSO-d₆) δ (ppm) 7.21 (s, 1H), 2.71 (t, J=7.3 Hz, 2H), 2.32 (s, 3H), 2.29 (s, 3H), 1.82 (t, J=7.3 Hz, 2H), 1.28 (s, 6H), 1.17 (s, 12H).

### Intermediate B-17

### 4,4,5,5-tetramethyl-2-(1,1,4-trimethylindan-5-yl)-1,3,2-dioxaborolane

Step1: 5-bromo-1,1,4-trimethyl-indane

In a round bottom flask, tetrachlorotitanium (20 mL of 1 M, 20 mmol) was dissolved in methylene chloride (25 mL) and cooled to −45° C. To the reaction, dimethylzinc (10 mL of 2 M, 20 mmol) was added dropwise, maintaining the temperature below −45° C. (internal temperature) and the reaction was stirred at −45° C. for 10 minutes. To the reaction mixture, a solution of 5-bromo-4-methyl-indan-1-one (2 g, 8.89 mmol) in methylene chloride (20 mL) was added dropwise at −45° C. then the reaction was gradually warmed to room temperature. The reaction was stirred at room temperature for 3 h and poured onto ice cold saturated sodium bicarbonate solution and acidified with concentrated HCl. The aqueous layer was extracted with DCM (3×), dried over magnesium sulfate, filtered and concentrated. The crude material was purified via silica gel column chromatography using hexanes to obtain 5-bromo-1,1,4-trimethyl-indane (1.45 g, 55%). ¹H-NMR (400 MHz, DMSO-d₆) δ (ppm) 7.37 (s, 1H), 6.93 (d, J=8.0 Hz, 1H), 2.84 (t, J=7.2 Hz, 2H), 2.25 (s, 3H), 1.87 (td, J=7.3, 1.2 Hz, 2H), 1.19 (d, J=1.2 Hz, 6H).

### Step 2: 4,4,5,5-tetramethyl-2-(1,1,4-trimethylindan-5-yl)-1,3,2-dioxaborolane (Intermediate B-17)

4,4,5,5-tetramethyl-2-(1,1,4-trimethylindan-5-yl)-1,3,2-dioxaborolane (Intermediate B-17) as a pale-yellow solid was prepared from 5-bromo-1,1,4-trimethyl-indane using procedure analogous to that found in Intermediate B-1, Step 2. ¹H NMR (400 MHz, DMSO-d₆) δ (ppm) 7.48 (d, J=7.6 Hz, 1H), 6.97 (d, J=7.5 Hz, 1H), 2.77 (t, J=7.2 Hz, 2H), 2.37 (s, 3H), 1.85 (t, J=7.2 Hz, 2H), 1.29 (s, 12H), 1.19 (s, 6H).

### Intermediate B-18

### 2-[5-methoxy-2-methyl-4-(2,2,2-trifluoro-1,1-dimethyl-ethyl)phenyl]-4,4,5,5-tetramethyl-1,3,2-dioxaborolane

### Step 1: 1-(4-bromo-2-hydroxy-5-methyl-phenyl)-2,2,2-trifluoro-ethanone

To a stirred solution of 3-bromo-4-methyl-phenol (2 g, 10.69 mmol) in dichloroethane (40 mL) at 0° C. was added dropwise trifluoroacetic anhydride (3.32 g, 2.2 mL, 15.82 mmol) over 5 minutes. Aluminum chloride (4.3 g, 32.25 mmol) was added to the reaction mixture portion-wise over 10 minutes. The reaction mixture was gradually warmed to room temperature over 2 h followed by heating at 40° C. for 16 h. The reaction mixture was cooled to room temperature and poured over ice water. The resultant mixture was extracted with DCM (2×30 mL). The combined organic layer was washed with a saturated aqueous solution of sodium bicarbonate (50 mL), saturated aqueous solution of sodium chloride (50 mL), dried over sodium sulfate and concentrated to dryness. The crude product was purified by reversed-phase chromatography (C₁₈) using 5 to 100% methanol in water (0.1% formic acid) to provide 1-(4-bromo-2-hydroxy-5-methyl-phenyl)-2,2,2-trifluoro-ethanone (1.21 g, 40%) as a light-yellow solid. ESI-MS m/z calc. 281.95, found 280.9 (M−1)⁻.

### Step 2: 1-(4-bromo-2-methoxy-5-methyl-phenyl)-2,2,2-trifluoro-ethanone

To a stirred solution of 1-(4-bromo-2-hydroxy-5-methyl-phenyl)-2,2,2-trifluoro-ethanone (695 mg, 2.45 mmol) in THF (7 mL) was added potassium carbonate (1 g, 7.24 mmol) followed by iodomethane (1.03 g, 0.45 mL, 7.23 mmol). The reaction mixture was heated at 68° C. for 16 h. The reaction mixture was cooled to room temperature and filtered through a plug of celite and washed with ethyl acetate (20 mL). The filtrate was concentrated under reduced pressure and purified by silica gel column chromatography using 0 to 5% ethyl acetate in heptanes to afford 1-(4-bromo-2-methoxy-5-methyl-phenyl)-2,2,2-trifluoro-ethanone (564 mg, 77%) as a white solid. ESI-MS m/z calc. 295.97, found 297.2 (M+1)⁺.

### Step 3: 2-(4-bromo-2-methoxy-5-methyl-phenyl)-1,1,1-trifluoro-propan-2-ol

To a stirred solution of 1-(4-bromo-2-methoxy-5-methyl-phenyl)-2,2,2-trifluoro-ethanone (560 mg, 1.88 mmol) in THF (8 mL) was added methylmagnesium bromide (3 M in diethyl ether) (2 mL of 3 M, 6 mmol) dropwise at 0° C. The reaction mixture was heated at 50° C. for 1 hour and cooled to 0° C. The reaction mixture was dropwise quenched with water (2 mL). A saturated solution of ammonium chloride (5 mL) was added, and the layers were separated. The aqueous layer was extracted with ethyl acetate (3×10 mL). The combined organic layer was washed with brine (15 mL), dried over anhydrous sodium sulfate, filtered and concentrated under reduced pressure. The crude product was purified by silica gel column chromatography using 0 to 5% ethyl acetate in heptanes to afford 2-(4-bromo-2-methoxy-5-methyl-phenyl)-1,1,1-trifluoro-propan-2-ol (531 mg, 89%) as a white solid. ESI-MS m/z calc. 311.99, found 295.2 (M−17)⁺. ¹H NMR (400 MHz, CDCl₃) δ (ppm) 7.18-7.16 (s, 1H), 7.16-7.14 (s, 1H), 5.85 (s, 1H), 3.92 (s, 3H), 2.36 (s, 3H), 1.74 (s, 3H). ¹⁹F NMR (377 MHz, CDCl₃) δ (ppm) −81.43 (s, 3F).

### Step 4: 1-bromo-4-(1-chloro-2,2,2-trifluoro-1-methyl-ethyl)-5-methoxy-2-methyl-benzene

To a mixture of 2-(4-bromo-2-methoxy-5-methyl-phenyl)-1,1,1-trifluoro-propan-2-ol (1 g, 3.19 mmol) and thionyl chloride (3.75 g, 2.3 mL, 31.53 mmol) was added pyridine (29.34 mg, 0.03 mL, 0.37 mmol) at room temperature. The reaction mixture was heated at 40° C. for 2 h. The reaction mixture was cooled to room temperature and poured onto a mixture of ice and a saturated aqueous solution of sodium bicarbonate (50 mL). The resulting mixture was extracted with DCM (3×15 mL), and the combined organic layer was washed with a saturated aqueous solution of sodium chloride (20 mL), dried over sodium sulfate, filtered and concentrated under reduced pressure to afford crude product 1-bromo-4-(1-chloro-2,2,2-trifluoro-1-methyl-ethyl)-5-methoxy-2-methyl-benzene (955 mg, 81%) as a clear oil. ¹H NMR (400 MHz, CDCl₃) δ (ppm) 7.56 (s, 1H), 7.13 (s, 1H), 3.85 (s, 3H), 2.36 (s, 3H), 2.25 (s, 3H). ¹⁹F NMR (377 MHz, CDCl₃) δ (ppm) −75.86 (s, 3F).

### Step 5: 1-bromo-5-methoxy-2-methyl-4-(2,2,2-trifluoro-1,1-dimethyl-ethyl)benzene

To a stirred solution of 1-bromo-4-(1-chloro-2,2,2-trifluoro-1-methyl-ethyl)-5-methoxy-2-methyl-benzene (955 mg, 2.5923 mmol) in DCM (20 mL) at −78° C. was added dropwise trimethylaluminum in heptanes (4.5 mL of 2 M, 9 mmol). The reaction mixture was stirred at this temperature for 30 minutes then warmed-up to room temperature and stirred overnight. The reaction mixture was cooled down to −78° C. and quenched dropwise with a saturated aqueous solution of sodium bicarbonate (10 mL). The aqueous layer was extracted with DCM (2×15 mL) and the combined organic layer was washed with a saturated aqueous solution of sodium chloride (25 mL), dried over sodium sulfate, filtered and concentrated under reduced pressure. The crude product was purified by silica gel column chromatography using heptanes to afford 1-bromo-5-methoxy-2-methyl-4-(2,2,2-trifluoro-1,1-dimethyl-ethyl)benzene (712 mg, 88%) as a white solid. ¹H NMR (400 MHz, CDCl₃) δ (ppm) 7.22 (s, 1H), 7.09 (s, 1H), 3.81 (s, 3H), 2.34 (s, 3H), 1.62 (s, 6H). ¹⁹F NMR (377 MHz, CDCl₃) δ (ppm) −74.85 (s, 3F).

### Step 6: 2-[5-methoxy-2-methyl-4-(2,2,2-trifluoro-1,1-dimethyl-ethyl)phenyl]-4,4,5,5-tetramethyl-1,3,2-dioxaborolane (Intermediate B-18)

2-[5-methoxy-2-methyl-4-(2,2,2-trifluoro-1,1-dimethyl-ethyl)phenyl]-4,4,5,5-tetramethyl-1,3,2-dioxaborolane (Intermediate B-18) was prepared from 1-bromo-5-methoxy-2-methyl-4-(2,2,2-trifluoro-1,1-dimethyl-ethyl)benzene using procedure analogous to that found in Intermediate B-1, Step 2 as an off-white solid. ESI-MS m/z calc. 358.19, found 359.2 (M+1)⁺. ¹H NMR (400 MHz, DMSO-d₆) δ (ppm) 7.21 (s, 1H), 7.17 (s, 1H), 3.77 (s, 3H), 2.40 (s, 3H), 1.60 (s, 6H), 1.30 (s, 12H). ¹⁹F NMR (377 MHz, DMSO-d₆) δ (ppm) −73.53 (s, 3F).

### Intermediate B-19

### 2-(4-(tert-butyl)-2-chlorophenyl)-4,4,5,5-tetramethyl-1,3,2-dioxaborolane

### Step 1: 2-(4-(tert-butyl)-2-chlorophenyl)-4,4,5,5-tetramethyl-1,3,2-dioxaborolane. (Intermediate B-19)

In a microwave vial a mixture of 1-bromo-4-tert-butyl-2-chloro-benzene (200 mg, 0.81 mmol), pinacolborane (1 g, 4 mmol), sodium acetate (80 mg, 0.1 mmol), Pd₂(dba)₃ (40 mg, 0.04 mmol) and XPhos (40 mg, 0.08 mmol) was sealed evacuated/backfilled with nitrogen and stirred neat at 110° C. for 6 h. The reaction mixture was filtered and concentrated to obtain 2-(4-(tert-butyl)-2-chlorophenyl)-4,4,5,5-tetramethyl-1,3,2-dioxaborolane (Intermediate B-19).

### Intermediate B-20

### 2-[4-(1-methoxy-1-methyl-ethyl)-2-methyl-phenyl]-4,4,5,5-tetramethyl-1,3,2-dioxaborolane

### Step 1: 2-(4-bromo-3-methyl-phenyl)propan-2-ol

In a 250-mL round-bottomed flask, methyl 4-bromo-3-methyl-benzoate (2.11 g, 8.76 mmol) was dissolved in THF (50 mL), to which a solution of methylmagnesium bromide in ether (7 mL of 3.0 M, 21 mmol) was added. The resulting reaction mixture was stirred under nitrogen at room temperature for 22 h. The reaction mixture was quenched with saturated aqueous ammonium chloride (100 mL). and extracted with ethyl acetate (3×100 mL). The combined organic layer was washed with water (100 mL) and brine (100 mL), dried over sodium sulfate, filtered, and concentrated in vacuo. Purification by silica gel chromatography using a gradient eluent of 0 to 40% ethyl acetate in hexanes gave 2-(4-bromo-3-methyl-phenyl)propan-2-ol (1.7185 g, 86%). ¹H NMR (400 MHz, CDCl₃) δ (ppm) 7.48 (d, J=8.3 Hz, 1H), 7.37 (dd, J=2.5, 0.8 Hz, 1H), 7.15 (ddd, J=8.3, 2.4, 0.6 Hz, 1H), 2.41 (s, 3H), 1.68 (s, 1H), 1.56 (s, 6H).

### Step 2: 1-bromo-4-(1-methoxy-1-methyl-ethyl)-2-methyl-benzene

In a 20-mL vial, 2-(4-bromo-3-methyl-phenyl)propan-2-ol (392 mg, 1.71 mmol) and iodomethane (200 μL, 3.21 mmol) were mixed with THF (3.0 mL), to which sodium hydride (106 mg of 60% w/w, 2.65 mmol) was added. The resulting mixture was stirred at room temperature for 24 h. The reaction mixture was diluted with ethyl acetate (10 mL) and washed with water (2×5 mL) and brine (5 mL), dried over sodium sulfate, filtered, and evaporated in vacuo to give 1-bromo-4-(1-methoxy-1-methyl-ethyl)-2-methyl-benzene (330 mg, 79%). ¹H NMR (400 MHz, CDCl₃) δ (ppm) 7.48 (d, J=8.3 Hz, 1H), 7.27 (d, J=2.4 Hz, 1H), 7.08 (dd, J=8.3, 2.4 Hz, 1H), 3.07 (s, 3H), 2.41 (s, 3H), 1.50 (s, 6H).

### Step 3: 2-[4-(1-methoxy-1-methyl-ethyl)-2-methyl-phenyl]-4,4,5,5-tetramethyl-1,3,2-dioxaborolane (Intermediate B-20)

In a 20-mL microwave vial, 1-bromo-4-(1-methoxy-1-methyl-ethyl)-2-methyl-benzene (330.0 mg, 1.36 mmol) was dissolved in dioxane (8 mL), to which triethylamine (600 μL, 4.30 mmol) and pinacolborane (600 μL, 4.14 mmol) were added. The reaction mixture was flushed with nitrogen, and Pd(dppf)Cl₂ (35 mg, 0.05 mmol) was added. The reaction mixture was subjected to microwave irradiation at 140° C. for 1 h. The reaction mixture was cooled to room temperature, diluted with ethyl acetate (15 mL), and slowly poured onto water (15 mL). The dark organic layer was separated, washed with brine, dried over sodium sulfate, filtered, and evaporated in vacuo. Purification by silica gel chromatography using 0 to 30% ethyl acetate in hexanes gave 2-[4-(1-methoxy-1-methyl-ethyl)-2-methyl-phenyl]-4,4,5,5-tetramethyl-1,3,2-dioxaborolane (Intermediate B-20, 221.0 mg, 56%); ¹H NMR (400 MHz, CDCl₃) δ (ppm) 7.75 (d, J=8.3 Hz, 1H), 7.23-7.17 (m, 2H), 3.06 (s, 3H), 2.55 (s, 3H), 1.51 (s, 6H), 1.34 (s, 12H).

### Intermediate B-21

### [5-chloro-2-methoxy-4-(trifluoromethyl)phenyl]boronic acid

### Step 1: 1-chloro-5-iodo-4-methoxy-2-(trifluoromethyl)benzene

To a solution of 1-chloro-4-methoxy-2-(trifluoromethyl)benzene (5 g, 23.74 mmol) in DCM (95 mL) was added iodine (6.63 g, 1.35 mL, 26.12 mmol), followed by the addition of silver triflate (7.32 g, 28.49 mmol). The reaction mixture was stirred at room temperature under and atmosphere of nitrogen for 1.5 h. The reaction mixture was filtered through celite and washed with DCM until the eluent was no longer purple. To the purple filtrate was added to a solution of aqueous sodium thiosulfate (50 mL). The biphasic mixture was diluted with water (100 mL) and the layers were separated. The aqueous layer was extracted with DCM (2×50 mL). The combined organics were dried over sodium sulfate, filtered and concentrated to give 1-chloro-5-iodo-4-methoxy-2-(trifluoromethyl)benzene (6 g, 75%). ¹H NMR (400 MHz, DMSO-d₆) δ (ppm); 8.08 (s, 1H), 7.29 (s, 1H), 3.89 (s, 3H).

### Step 2: [5-chloro-2-methoxy-4-(trifluoromethyl)phenyl]boronic acid (Intermediate B-21)

To a solution of 1-chloro-5-iodo-4-methoxy-2-(trifluoromethyl)benzene (500 mg, 1.49 mmol) in anhydrous THF (5 mL) under an atmosphere of nitrogen at −78° C. was added isopropylmagnesium chloride (1.2 mL of 2 M, 2.38 mmol) over a period of 5 minutes. The reaction mixture was stirred for 1 h, gradually warmed to −55° C. and triisopropylborate (391.26 mg, 0.48 mL, 2.08 mmol) was added. The reaction was stirred for 1 h and quenched with saturated ammonium chloride solution (50 mL). The aqueous layer was extracted with ethyl acetate (2×50 mL). The combined organic layer was washed with brine solution (20 mL) dried over sodium sulfate filtered and concentrated to obtain [5-chloro-2-methoxy-4-(trifluoromethyl)phenyl]boronic acid (Intermediate B-21, 200 mg, 53%) as a white solid. ¹H NMR (400 MHz, DMSO-d₆) δ (ppm); 8.18 (s, 1H), 7.60 (s, 1H), 7.28 (s, 1H), 6.51 (s, 1H), 3.85 (s, 3H).

### Intermediate B-22

### 2-(4-tert-butyl-2,6-dimethyl-phenyl)-4,4,5,5-tetramethyl-1,3,2-dioxaborolane

### Step 1: 2-bromo-5-tert-butyl-1,3-dimethyl-benzene

Under an inert atmosphere, acetic acid (30 mL) was added to a 100 mL three-necked flask and 1-tert-butyl-3,5-dimethyl-benzene (3 g, 18.49 mmol) was added. Bromine (1.25 mL, 24.26 mmol) was added, and the reaction mixture was stirred at room temperature for 3 h. The obtained reaction solution was added to the water (500 ml) and the deposited precipitate was filtered and washed with water (250 ml). The residue was taken up in DCM and dried over magnesium sulfate, filtered and concentrated. The crude material was purified via silica gel column chromatography using 0 to 30% ethyl acetate in hexanes to obtain 2-bromo-5-tert-butyl-1,3-dimethyl-benzene (3.97 g, 89%). ¹H NMR (500 MHz, DMSO-d₆) δ (ppm) 7.19 (s, 2H), 2.35 (s, 6H), 1.26 (s, 9H).

### Step 2: 2-(4-tert-butyl-2,6-dimethyl-phenyl)-4,4,5,5-tetramethyl-1,3,2-dioxaborolane (Intermediate B-22)

2-(4-tert-butyl-2,6-dimethyl-phenyl)-4,4,5,5-tetramethyl-1,3,2-dioxaborolane (Intermediate B-22) was prepared from 2-bromo-5-tert-butyl-1,3-dimethyl-benzene using procedure analogous to that found in Intermediate B-1, Step 2. ¹H NMR (500 MHz, DMSO-d₆) δ (ppm) 6.96 (s, 2H), 2.31 (s, 6H), 1.32 (s, 12H), 1.24 (s, 9H).

### Intermediate B-23

### 2-tert-butyl-6-methyl-5-(4,4,5,5-tetramethyl-1,3,2-dioxaborolan-2-yl)-3-(trifluoromethyl)pyridine

### Step 1: 6-tert-butyl-2-hydroxy-5-iodo-pyridine-3-carbonitrile

To a solution of 6-tert-butyl-2-hydroxy-pyridine-3-carbonitrile (4.7 g, 26.67 mmol) in DCE (90 mL) and TFA (30 mL) was added N-Iodosuccinimide (15 g, 66.67 mmol) and the reaction mixture was stirred at 50° C. overnight. The reaction mixture was concentrated under reduced pressure and ethyl acetate (150 mL) was added. The organic layer was washed with an aqueous solution of 10% sodium thiosulfate (150 mL), water (2×150 mL) and brine (150 mL). The organic layer was dried over anhydrous sodium sulfate, filtered and evaporated under reduced pressure. The residue was purified by silica gel chromatography using 0 to 60% ethyl acetate in heptane affording 6-tert-butyl-2-hydroxy-5-iodo-pyridine-3-carbonitrile (6.7 g, 78%) as a yellow solid. ESI-MS m/z calc. 301.99, found 303.0 (M+1)⁺. ¹H NMR (400 MHz, CDCl₃) δ (ppm) 8.18 (s, 1H), 2.63 (s, 1H), 1.58 (s, 9H, overlapped with water).

### Step 2: 6-tert-butyl-2-hydroxy-5-(trifluoromethyl)pyridine-3-carbonitrile

To a solution of 6-tert-butyl-2-hydroxy-5-iodo-pyridine-3-carbonitrile (200 mg, 0.62 mmol), CuI (360 mg, 1.89 mmol) and potassium fluoride (110 mg, 1.89 mmol) in NMP (1.5 mL) was added methyl 2,2-difluoro-2-fluorosulfonyl-acetate (603.60 mg, 0.4 mL, 3.14 mmol) at room temperature under nitrogen atmosphere. The mixture was then stirred at 120° C. for 24 h. It was cooled to room temperature, diluted with water (100 mL) and ethyl acetate (100 mL). The layers were separated, and the aqueous layer was extracted with ethyl acetate (50 mL). The combined organic layers were then washed with water (50 mL), brine (70 mL), dried over anhydrous sodium sulfate, filtered and concentrated. The crude product was purified by reversed-phase column chromatography (C₁₈) using 2 to 100% acetonitrile in water with 0.1% formic acid to afford 6-tert-butyl-2-hydroxy-5-(trifluoromethyl)pyridine-3-carbonitrile (30 mg, 20%) as an orange solid. ESI-MS m/z calc. 244.08, found 245.2 (M+1)⁺. ¹H NMR (400 MHz, CDCl₃) δ (ppm) 9.41 (br s, 1H), 8.12 (s, 1H), 1.53 (s, 9H). ¹⁹F NMR (377 MHz, CDCl₃) δ (ppm) −52.63 (s, 3F).

### Step 3: 2-bromo-6-tert-butyl-5-(trifluoromethyl)pyridine-3-carbonitrile

To a stirred suspension of 6-tert-butyl-2-hydroxy-5-(trifluoromethyl)pyridine-3-carbonitrile (1.67 g, 6.83 mmol) in toluene (40 mL) was added phosphorus oxybromide (2.75 g, 9.59 mmol). The reaction mixture was stirred at 95° C. overnight. Additional phosphorus oxybromide (2 g, 6.98 mmol) was added and the reaction mixture stirred for 24 h. at 95° C. The reaction mixture was cooled to room temperature and quenched by slow addition of saturated sodium bicarbonate solution (150 mL) and water (200 mL). The aqueous layer was extracted with ethyl acetate (2×200 mL). The combined organic layers were washed with brine (100 mL), dried over sodium sulfate, filtered and concentrated under reduced pressure. The crude product was purified by silica gel flash chromatography using 0 to 10% ethyl acetate in heptanes to afford 2-bromo-6-tert-butyl-5-(trifluoromethyl)pyridine-3-carbonitrile (1.70 g, 77%) as a brown oil. ESI-MS m/z calc. 305.99, found 307.0 (M+1)⁺.

### Step 4: 6-tert-butyl-2-methyl-5-(trifluoromethyl)pyridine-3-carbonitrile

A solution of 2-bromo-6-tert-butyl-5-(trifluoromethyl)pyridine-3-carbonitrile (1.6 g, 4.98 mmol) in 1,4-dioxane (15 mL) was sparged with nitrogen for 10 min then trimethylboroxine (1.12 g, 1.25 mL, 8.94 mmol), potassium carbonate (2.06 g, 14.90 mmol) and Pd(dppf)₂Cl₂·DCM (205 mg, 0.25 mmol) were added. The resulting mixture was stirred for 20 h. at 100° C. The resulting reaction mixture was filtered over celite and rinsed with acetonitrile (100 mL). The filtrate was evaporated under reduced pressure and purified by silica gel flash chromatography using 0 to 10% ethyl acetate in heptanes to afford 6-tert-butyl-2-methyl-5-(trifluoromethyl)pyridine-3-carbonitrile (1 g, 80%) as a colorless oil. ESI-MS m/z calc. 242.10, found 243.2 (M+1)⁺. ¹H NMR (400 MHz, CDCl₃) δ (ppm) 8.16 (s, 1H), 2.79 (s, 3H), 1.46 (s, 9H). ¹⁹F NMR (377 MHz, CDCl₃) δ (ppm) −54.65 (s, 3F).

### Step 5: 6-tert-butyl-2-methyl-5-(trifluoromethyl)pyridine-3-carboxylic acid

To a solution of 6-tert-butyl-2-methyl-5-(trifluoromethyl)pyridine-3-carbonitrile (1 g, 3.99 mmol) in ethanol (30 mL) was added an aqueous solution of NaOH (5 mL of 20 M, 100 mmol). The reaction mixture was then stirred at 100° C. overnight. The reaction mixture was cooled down to room temperature and solvent was removed under reduced pressure, the residue was diluted with water (100 mL) and an aqueous solution of 6 M HCl was added (pH ˜5/6). The aqueous mixture was diluted with ethyl acetate (100 mL). The layers were separated, and the aqueous layer was extracted with ethyl acetate (2×100 mL). The combined organic layer was washed with brine (150 mL), dried over sodium sulfate, filtered and concentrated under reduced pressure. The crude product was purified by reversed-phase column chromatography (C₁₈) using 5 to 100% acetonitrile in water with 0.1% formic acid to afford 6-tert-butyl-2-methyl-5-(trifluoromethyl)pyridine-3-carboxylic acid (930 mg, 89%) as a tan solid. ESI-MS m/z calc. 261.10, found 262.2 (M+1)⁺. ¹H NMR (400 MHz, DMSO-d₆) δ (ppm) 13.59 (br. s., 1H), 8.42 (s, 1H), 2.78 (s, 3H), 1.42 (s, 9H). ¹⁹F NMR (377 MHz, DMSO-d₆) δ (ppm) −53.28 (s, 3F).

### Step 6: 5-bromo-2-tert-butyl-6-methyl-3-(trifluoromethyl)pyridine

A suspension of 6-tert-butyl-2-methyl-5-(trifluoromethyl)pyridine-3-carboxylic acid (480 mg, 1.80 mmol), anhydrous potassium phosphate tribasic (770 mg, 3.63 mmol), tetrabutylammonium tribromide (5.22 g, 10.83 mmol) and anhydrous acetonitrile (10 mL) was refluxed at 100° C. for 3 days. Another portion of tetrabutylammonium tribromide (2.61 g, 5.41 mmol) was added and the reaction mixture was stirred at 100° C. for 20 hours. Once cooled to room temperature, the reaction mixture was filtrated through celite and rinsed with ethyl acetate (100 mL). The resulting material was concentrated under reduced pressure and purified by reversed-phase flash chromatography (C₁₈) using 5 to 100% acetonitrile in water with 0.1% formic acid to afford 5-bromo-2-tert-butyl-6-methyl-3-(trifluoromethyl)pyridine (198 mg, 28%).

### Step 7: 2-tert-butyl-6-methyl-5-(4,4,5,5-tetramethyl-1,3,2-dioxaborolan-2-yl)-3-(trifluoromethyl)pyridine (Intermediate B-23)

2-tert-butyl-6-methyl-5-(4,4,5,5-tetramethyl-1,3,2-dioxaborolan-2-yl)-3-(trifluoromethyl)pyridine (Intermediate B-23) as a colorless oil was prepared from 5-bromo-2-tert-butyl-6-methyl-3-(trifluoromethyl)pyridine using procedure analogous to that found in Intermediate B-1, Step 2. ESI-MS m/z calc. 343.19, found 344.2 (M+1)⁺. ¹H NMR (400 MHz, CDCl₃) δ (ppm) 8.27 (s, 1H), 2.73 (s, 3H), 1.45-1.43 (m, 9H), 1.35 (s, 12H). ¹⁹F NMR (377 MHz, CDCl₃) δ (ppm) −53.90 (s, 3F).

### Intermediate B-24

### 2-(4-tert-butyl-2,5-dimethyl-phenyl)-4,4,5,5-tetramethyl-1,3,2-dioxaborolane

### Step 1: 2-(4-tert-butyl-2,5-dimethyl-phenyl)-4,4,5,5-tetramethyl-1,3,2-dioxaborolane (Intermediate B-24)

To a solution of 4-tert-butyl-2,5-dimethyl-phenol (2.568 g, 14.41 mmol) in DCM (29 mL) was added pyridine (2.33 mL, 28.81 mmol) and the reaction mixture was cooled to 0° C. Trifluoromethanesulfonate (2.9 mL, 17.24 mmol) was added dropwise, and the reaction was gradually warmed to room temperature. After stirring at room temperature for 1.5 h., the reaction mixture was diluted with ether and washed with 1N HCl. The organic layer was further washed with saturated sodium bicarbonate solution (3×) and brine. The organic layer was isolated, dried over magnesium sulfate, filtered, and evaporated to dryness. The crude material was purified by silica gel column chromatography using 0 to 10% ethyl acetate in hexanes to obtain triflate, (4-tert-butyl-2,5-dimethyl-phenyl)trifluoromethanesulfonate.

In a reaction vial, the intermediate from step 1, (4-tert-butyl-2,5-dimethyl-phenyl)trifluoromethanesulfonate (4.223 g), was mixed with triethylamine (5.7 mL, 40.82 mmol) and pinacolborane (5.9 mL, 40.83 mmol) in dioxane (68 mL). The reaction mixture was purged with nitrogen and PdCl₂(dppf) (300 mg, 0.41 mmol) was added. The reaction mixture was refluxed overnight. The reaction was quenched with water and extracted with ethyl acetate. The layers were separated, and the organic layer was washed with brine, dried over sodium sulfate, filtered, and concentrate. Purification by silica gel column chromatography using 5 to 20% ethyl acetate in hexanes gave 2-(4-tert-butyl-2,5-dimethyl-phenyl)-4,4,5,5-tetramethyl-1,3,2-dioxaborolane (Intermediate B-24, 2.31 g, 56%) as a white solid. ¹H-NMR (400 MHz, CDCl₃) δ (ppm) 7.52 (s, 1H), 7.17 (s, 1H), 2.51 (s, 3H), 2.50 (s, 3H), 1.39 (s, 9H), 1.32 (s, 12H).

### Intermediate B-25

### 4,4,5,5-tetramethyl-2-(1,1,6-trimethylindan-5-yl)-1,3,2-dioxaborolane

### Step 1: 1,1,6-trimethylindan-5-ol

To a solution of 6-bromo-5-methoxy-1,1-dimethyl-indane (957 mg, 3.71 mmol) in dioxane (9.6 mL) was added methylboronic acid (445 mg, 7.43 mmol) and Potassium carbonate (1.54 g, 11.14 mmol). The reaction mixture was purged with nitrogen and PdCl₂(dppf) (272 mg, 0.37 mmol) was added. The reaction was heated at 100° C. and stirred for 16 h. The reaction mixture was cooled to room temperature, diluted with ethyl acetate, and filtered through celite. The reaction mixture was then washed with water, brine, dried over sodium sulfate, filtered and concentrated. The crude material was purified by silica gel column chromatography using 0 to 5% ethyl acetate in hexanes. In a 100 mL round bottom flask, the intermediate from step 1, 5-methoxy-1,1,6-trimethyl-indane (612 mg), was dissolved in methylene chloride (41 mL) and cooled to −78° C. Boron tribromide (6.44 mL of 1 M, 6.44 mmol) was added dropwise, and the reaction mixture was gradually warmed to room temperature and stirred for 2 h. The reaction was then quenched with methanol (7 mL) followed by saturated sodium bicarbonate solution. (14 mL). The reaction mixture was stirred at room temperature for 1 h. then extracted with DCM. The combined organic layer was washed with brine, dried over magnesium sulfate, filtered, and evaporated to dryness. The crude material was purified by silica gel column chromatography using 5 to 25% ethyl acetate in hexanes gradient to obtain 1,1,6-trimethylindan-5-ol (458.3 mg, 69%). ¹H NMR (400 MHz, CDCl₃) δ (ppm) 6.87 (s, 1H), 6.62 (s, 1H), 4.48 (s, 1H), 2.80 (t, J=7.2 Hz, 2H), 2.23 (s, 3H), 1.89 (t, J=7.2 Hz, 2H), 1.22 (s, 6H).

### Step 2: 4,4,5,5-tetramethyl-2-(1,1,6-trimethylindan-5-yl)-1,3,2-dioxaborolane (Intermediate B-25)

4,4,5,5-tetramethyl-2-(1,1,6-trimethylindan-5-yl)-1,3,2-dioxaborolane (Intermediate B-25) was prepared from 1,1,6-trimethylindan-5-ol using procedure analogous to that found in Intermediate B-24, Step 1 as a white solid. ¹H NMR (400 MHz, CDCl₃) δ (ppm) 7.63 (s, 1H), 6.95 (s, 1H), 2.84 (t, J=7.1 Hz, 2H), 2.53 (s, 3H), 1.88 (t, J=7.2 Hz, 2H), 1.32 (s, 12H), 1.23 (s, 6H).

### Intermediate B-26

### 4,4,5,5-tetramethyl-2-[2-methyl-4-(2,2,2-trifluoro-1,1-dimethyl-ethyl)phenyl]-1,3,2-dioxaborolane

### Step 1: 1,1,1-trifluoro-2-(4-methoxy-3-methyl-phenyl)propan-2-ol

To a solution of 2,2,2-trifluoro-1-(4-methoxy-3-methyl-phenyl)ethanone (1.7 g, 7.79 mmol) in tetrahydrofuran (28 mL) cooled to 0° C. was slowly added methylmagnesium bromide (7.8 mL of 3 M, 23.4 mmol) as a solution in diethyl ether and the reaction mixture was stirred at 50° C. for 2 hours. The mixture was cooled to 0° C. and quenched slowly with water and a saturated solution of ammonium chloride. The layers were separated, and the aqueous layer was extracted with ethyl acetate (3×25 mL). The combined organic layer was washed with brine, dried over anhydrous magnesium sulfate, filtered, concentrated in vacuo and dried under high vacuum to provide 1,1,1-trifluoro-2-(4-methoxy-3-methyl-phenyl)propan-2-ol (1.81 g, 98%) as a light orange oil. ESI-MS m/z calc. 234.09, found 216.4 (M−18)⁺. H NMR (400 MHz, DMSO-d₆) δ (ppm) 7.37 (s, 1H), 7.35 (s, 1H), 6.92 (d, J=8.3 Hz, 1H), 6.40 (s, 1H), 3.78 (s, 3H), 2.16 (s, 3H), 1.64 (s, 3H). ¹⁹F NMR (377 MHz, DMSO-d₆) δ (ppm) −79.85 (s, 3F).

### Step 2: 1-methoxy-2-methyl-4-(2,2,2-trifluoro-1,1-dimethyl-ethyl)benzene

To a solution of 1,1,1-trifluoro-2-(4-methoxy-3-methyl-phenyl)propan-2-ol (2.7 g, 11.53 mmol) in DCM (108 mL) cooled to 0° C. was added a solution of titanium(IV) chloride (11.9 mL of 1 M, 11.9 mmol) in toluene and the reaction mixture was stirred at the same temperature for 2 h. Ice-cold water and DCM were added, and the layers were separated. The aqueous layer was extracted with DCM (2×100 mL). The combined organic layer was washed with a saturated solution of sodium bicarbonate, dried over anhydrous magnesium sulfate, filtered and concentrated in vacuo. The residue was dissolved in DCM (108 mL) and cooled to −70° C. and a solution of titanium(IV) chloride (11.9 mL of 1 M, 11.9 mmol) in toluene was added dropwise, followed by dropwise addition of a solution of dimethylzinc (9.8 mL of 2 M, 19.6 mmol) in toluene. The mixture was gradually warmed to room temperature and stirred for 68 h. Ice-cold water and DCM were added, and the reaction mixture was filtered over celite. The layers were separated, and the aqueous layer was extracted with DCM (2×50 mL). The combined organic layer was washed with a saturated solution of sodium bicarbonate, dried over anhydrous magnesium sulfate, filtered, concentrated in vacuo and dried under high vacuum to provide crude 1-methoxy-2-methyl-4-(2,2,2-trifluoro-1,1-dimethyl-ethyl)benzene (2.55 g, 44%) as an orange oil. ¹H NMR (400 MHz, CDCl₃) δ (ppm) 7.32-7.24 (m, 2H), 6.84-6.78 (d, J=8.8 Hz, 1H), 3.84 (s, 3H), 2.24 (s, 3H), 1.56 (s, 6H). ¹⁹F NMR (377 MHz, CDCl₃) δ (ppm) −76.41 (s, 3F).

### Step 3: 2-methyl-4-(2,2,2-trifluoro-1,1-dimethyl-ethyl)phenol

To a solution of 1-methoxy-2-methyl-4-(2,2,2-trifluoro-1,1-dimethyl-ethyl)benzene (2.18 g, 5.39 mmol) in DCM (34 mL) cooled to 0° C. was added a solution of boron tribromide (5.4 mL of 1 M, 5.4 mmol) in DCM dropwise. The reaction mixture was warmed to room temperature and stirred for 1 h. The solution was cooled again to 0° C. and an additional solution of boron tribromide (5.4 mL of 1 M, 5.4 mmol) in DCM was added. After stirring for 16 h at room temperature, the reaction mixture was again cooled to 0° C., and a solution of boron tribromide (2.7 mL of 1 M, 2.7 mmol) in DCM was added. The mixture was warmed to room temperature and stirred for 0.5 h. The reaction mixture was cooled down to 0° C. and quenched by slow addition of water (50 mL). The layers were separated, and the aqueous layer was extracted with DCM (3×50 mL). The combined organic layer was washed with brine, dried over anhydrous magnesium sulfate and concentrated in vacuo. Purification by silica gel chromatography using 0 to 5% ethyl acetate in heptanes followed by a second purification using reverse phase chromatography (C₁₈) using 5 to 100% methanol in water with 0.1% formic acid provided 2-methyl-4-(2,2,2-trifluoro-1,1-dimethyl-ethyl)phenol (1.21 g, 99%) as a brown oil. ESI-MS m/z calc. 218.09, found 219.1 (M+1)⁺. ¹H NMR (400 MHz, CDCl₃) δ (ppm) 7.27-7.17 (m, 2H), 6.76 (d, J=8.3 Hz, 1H), 4.84 (br. s, 1H), 2.28 (s, 3H), 1.55 (s, 6H). ¹⁹F NMR (377 MHz, CDCl₃) δ (ppm) −76.43 (s, 3F).

### Step 4: [2-methyl-4-(2,2,2-trifluoro-1,1-dimethyl-ethyl)phenyl] trifluoromethanesulfonate

To a solution of 2-methyl-4-(2,2,2-trifluoro-1,1-dimethyl-ethyl)phenol (1.21 g, 5.3564 mmol) and pyridine (850.86 mg, 0.87 mL, 10.76 mmol) in DCM (25 mL) cooled at −50° C. under an atmosphere of nitrogen was added trifluoromethanesulfonic anhydride (2 g, 1.2 mL, 7.1 mmol) dropwise. The reaction mixture was warmed to room temperature over 1 hour. The reaction mixture was washed with water (25 mL) and 1N aqueous HCl (25 mL), dried over anhydrous magnesium sulfate, filtered and concentrated in vacuo. Purification by silica gel chromatography using 0 to 20% ethyl acetate in heptanes provided [2-methyl-4-(2,2,2-trifluoro-1,1-dimethyl-ethyl)phenyl] trifluoromethanesulfonate (1.68 g, 89%) as a yellow oil. ¹H NMR (400 MHz, CDCl₃) δ (ppm) 7.44-7.36 (m, 2H), 7.23 (d, J=8.6 Hz, 1H), 2.41 (s, 3H), 1.58 (s, 6H). ¹⁹F NMR (377 MHz, CDCl₃) δ (ppm) −73.85 (s, 3F), −76.15 (s, 3F).

### Step 5: 4,4,5,5-tetramethyl-2-[2-methyl-4-(2,2,2-trifluoro-1,1-dimethyl-ethyl)phenyl]-1,3,2-dioxaborolane (Intermediate B-26)

To a solution of [2-methyl-4-(2,2,2-trifluoro-1,1-dimethyl-ethyl)phenyl]trifluoromethanesulfonate (5.94 g, 16.941 mmol) in 1,4-dioxane (35 mL) were added bis(pinacolato)diboron (5.2 g, 20.47 mmol) and potassium acetate (5 g, 50.95 mmol). The reaction mixture was degassed with nitrogen for 10 minutes and PdCl₂(dppf) DCM (1.4 g, 1.71 mmol) was added and the resulting reaction mixture was degassed under nitrogen for an additional 10 minutes. The tube was sealed and heated at 120° C. for 2 h. The mixture was cooled to room temperature, diluted with ethyl acetate (50 mL) and filtered through celite. A saturated solution of ammonium chloride (25 mL) was added, and the layers were separated. The aqueous layer was extracted with ethyl acetate (3×50 mL). The combined organic layer was washed with brine, dried over anhydrous magnesium sulfate, filtered and concentrated in vacuo. Purification by silica gel chromatography using heptanes provided 4,4,5,5-tetramethyl-2-[2-methyl-4-(2,2,2-trifluoro-1,1-dimethyl-ethyl)phenyl]-1,3,2-dioxaborolane (Intermediate B-26, 4.42 g, 79%) as a white powder. ESI-MS m/z calc. 328.18, found 329.2 (M+1)⁺. ¹H NMR (400 MHz, DMSO-d₆) δ (ppm) 7.63 (d, J=7.8 Hz, 1H), 7.35-7.29 (m, 2H), 2.48 (s, 3H), 1.53 (s, 6H), 1.29 (s, 12H). ¹⁹F NMR (377 MHz, DMSO-d₆) δ (ppm) −74.75 (s, 3F).

### Intermediate B-27

### 4,4,5,5-tetramethyl-2-(1,1,7-trimethyltetralin-6-yl)-1,3,2-dioxaborolane

### Step 1: 6-methoxy-1,1,7-trimethyl-tetralin

To a flame-dried flask was added titanium(IV) chloride in toluene (19.5 mL of 1 M, 19.5 mmol) in DCM (30 mL) and the reaction mixture was cooled to −45° C. A solution of dimethylzinc (1.2 M in toluene) (25.5 mL of 1.2 M, 30.6 mmol) was added to the reaction mixture at this temperature (−50 to −45° C.). After stirring for 15 minutes, a solution of 6-methoxy-7-methyl-tetralin-1-one (2 g, 10.51 mmol) in DCM (15 mL) was added dropwise at −45° C. The reaction mixture was gradually warmed to room temperature and stirred for 18 hours. The reaction mixture was poured onto ice water and extracted with DCM (3×20 mL). The combined organic layer was washed with a saturated aqueous solution of sodium chloride, dried over sodium sulfate, filtered and concentrated under reduced pressure. Purification by silica gel column chromatography using 0 to 25% ethyl acetate in heptanes gave 6-methoxy-1,1,7-trimethyl-tetralin (1.52 g, 71%) as a clear oil. ¹H NMR (400 MHz, CDCl₃) δ (ppm) 7.08 (s, 1H), 6.49 (s, 1H), 3.79 (s, 3H), 2.74 (t, J=6.4 Hz, 2H), 2.19 (s, 3H), 1.85-1.76 (m, 2H), 1.68-1.61 (m, 2H), 1.26 (s, 6H).

### Step 2: 1,1,7-trimethyltetralin-6-ol

To a stirred solution of 6-methoxy-1,1,7-trimethyl-tetralin (1.5 g, 7.33 mmol) in DCM (15 mL) at −78° C. was added a solution of boron tribromide (1M in DCM) (15 mL of 1 M, 15 mmol) dropwise. The reaction mixture warmed to room temperature and stirred for 2 hours. The reaction mixture was quenched with methanol (10 mL) followed by a saturated aqueous solution of sodium bicarbonate (15 mL). The aqueous layer was extracted with DCM (3×20 mL) and the combined organic layer was washed with a saturated aqueous solution of sodium chloride, dried over sodium sulfate, filtered and concentrated under reduced pressure. Purification by silica gel column chromatography using 0 to 35% ethyl acetate in heptanes gave 1,1,7-trimethyltetralin-6-ol (1.22 g, 87%) as a white solid. ESI-MS m/z calc. 190.13, found 191.2 (M+1)⁺. ¹H NMR (400 MHz, CDCl₃) δ (ppm) 7.06 (s, 1H), 6.46 (s, 1H), 4.43 (s, 1H), 2.68 (t, J=6.4 Hz, 2H), 2.22 (s, 3H), 1.82-1.74 (m, 2H), 1.66-1.60 (m, 2H), 1.26 (s, 6H).

### Step 3: 4,4,5,5-tetramethyl-2-(1,1,7-trimethyltetralin-6-yl)-1,3,2-dioxaborolane (Intermediate B-27)

4,4,5,5-tetramethyl-2-(1,1,7-trimethyltetralin-6-yl)-1,3,2-dioxaborolane (Intermediate B-27) was prepared from 1,1,7-trimethyltetralin-6-ol using procedure analogous to that found in Intermediate B-24, Step 1. ¹H NMR (400 MHz, CDCl₃) δ (ppm) 7.47 (s, 1H), 7.12 (s, 1H), 2.73 (t, J=6.3 Hz, 2H), 2.49 (s, 3H), 1.81-1.73 (m, 2H), 1.66-1.61 (m, 2H), 1.32 (s, 12H), 1.26 (s, 6H).

### Intermediate B-28

### 4,4,5,5-tetramethyl-2-[2-methyl-4-(1-methylcyclobutyl)phenyl]-1,3,2-dioxaborolane

### Step 1: (4-bromo-2-methyl-phenoxy)-tert-butyl-dimethyl-silane

To a solution of 4-bromo-2-methylphenol (1 g, 5.35 mmol) in DCM (10 mL) were added imidazole (550 mg, 8.08 mmol) and tert-butyldimethylsilyl chloride (890 mg, 5.91 mmol). The reaction mixture was stirred for 1.5 h. at room temperature and filtered. The filtrate was washed with an aqueous 0.5 N hydrochloric acid (2×30 mL), brine, dried over sodium sulfate, filtered and concentrated under reduced pressure. Purification by silica gel flash chromatography using 100% Heptanes afforded (4-bromo-2-methyl-phenoxy)-tert-butyl-dimethyl-silane (1.36 g, 84%) as a colorless oil. H NMR (400 MHz, CDCl₃) δ (ppm) 7.26 (d, J=2.0 Hz, 1H), 7.16 (dd, J=8.6, 2.1 Hz, 1H), 6.64 (d, J=8.6 Hz, 1H), 2.18 (s, 3H), 1.02 (s, 9H), 0.21 (s, 6H).

### Step 2: 1-[4-[tert-butyl(dimethyl)silyl]oxy-3-methyl-phenyl]cyclobutanol

To a solution of (4-bromo-2-methyl-phenoxy)-tert-butyl-dimethyl-silane (1.35 g, 4.48 mmol) in THF (45 mL) cooled to −78° C. was slowly added n-BuLi in hexanes (1.9 mL of 2.5 M, 4.75 mmol) under an atmosphere of nitrogen. The reaction mixture was stirred at −78° C. for 1 h and cyclobutanone (347 mg, 0.37 mL, 4.95 mmol) was added. The reaction mixture was stirred at −78° C. for 5 h and quenched by addition of a saturated aqueous ammonium chloride solution (50 mL). The aqueous layer was extracted with ethyl acetate (2×75 mL). The combined organic layer was washed with brine, dried over sodium sulfate, filtered and concentrated under reduced pressure. Purification by silica gel chromatography using 0 to 10% ethyl acetate in heptanes gave 1-[4-[tert-butyl(dimethyl)silyl]oxy-3-methyl-phenyl]cyclobutanol (1.02 g, 77%). ESI-MS m/z calc. 292.19, found 275.3 (M−17)⁺. ¹H NMR (400 MHz, DMSO-d₆) δ (ppm) 7.24 (d, J=1.7 Hz, 1H), 7.16 (dd, J=8.3, 2.0 Hz, 1H), 6.73 (d, J=8.3 Hz, 1H), 5.29 (s, 1H), 2.39-2.29 (m, 2H), 2.25-2.17 (m, 2H), 2.15 (s, 3H), 1.92-1.80 (m, 1H), 1.64-1.51 (m, 1H), 0.99 (s, 9H), 0.19 (s, 6H).

### Step 3: tert-butyl-dimethyl-[2-methyl-4-(1-methylcyclobutyl)phenoxy]silane

To a solution of 1-[4-[tert-butyl(dimethyl)silyl]oxy-3-methyl-phenyl]cyclobutanol (800 mg, 2.69 mmol) in DCM (12 mL) cooled to −78° was added titanium(IV) chloride (in DCM) (6 mL of 1 M, 6 mmol) dropwise under an atmosphere of nitrogen. The resulting mixture was stirred at −78° C. for 1 h. and dimethylzinc (in heptanes) (8 mL of 1 M, 8 mmol) was added slowly and the resulting mixture was stirred at −78° C. for 2 h. The reaction mixture was warmed to room temperature and poured onto cold water (50 mL) while stirring vigorously. The resulting suspension was diluted with DCM (50 mL), stirred for 5 minutes, and filtered through celite. The layers were separated and the organic layer was washed with brine (20 mL), dried over sodium sulfate, filtered, and concentrated under reduced pressure to afford tert-butyl-dimethyl-[2-methyl-4-(1-methylcyclobutyl)phenoxy]silane (775 mg, 87%) (88% purity). ¹H NMR (400 MHz, CDCl₃) δ (ppm) 6.93 (d, J=1.7 Hz, 1H), 6.85 (dd, J=8.3, 2.0 Hz, 1H), 6.69 (d, J=8.2 Hz, 1H), 2.41-2.30 (m, 2H), 2.20 (s, 3H), 2.13-2.05 (m, 1H), 2.05-1.97 (m, 2H), 1.87-1.76 (m, 1H), 1.43 (s, 3H), 1.01 (s, 9H), 0.22 (s, 6H).

### Step 4: 2-methyl-4-(1-methylcyclobutyl)phenol

To a solution of tert-butyl-dimethyl-[2-methyl-4-(1-methylcyclobutyl)phenoxy]silane (965 mg, 3.12 mmol) in THF (9 mL) was added tetrabutylammonium fluoride (in THF) (3.5 mL of 1 M, 3.5 mmol). The resulting mixture was stirred at room temperature for 2 h and quenched with saturated ammonium chloride solution (25 mL). Ethyl acetate (100 mL) was added, and the layers were separated. The aqueous layer was washed with water (3×75 mL), brine, dried over anhydrous sodium sulfate, filtered and concentrated under reduced pressure. Purification by silica gel chromatography using 5 to 30% ethyl acetate in heptanes gave 2-methyl-4-(1-methylcyclobutyl)phenol (428 mg, 71%). ¹H NMR (400 MHz, CDCl₃) δ (ppm) 6.92 (s, 1H), 6.91-6.87 (m, 1H), 6.72 (d, J=8.1 Hz, 1H), 4.52 (br. s, 1H), 2.41-2.30 (m, 2H), 2.26 (s, 3H), 2.14-1.98 (m, 3H), 1.87-1.77 (m, 1H), 1.43 (s, 3H).

### Step 5: 4,4,5,5-tetramethyl-2-[2-methyl-4-(1-methylcyclobutyl)phenyl]-1,3,2-dioxaborolane (Intermediate B-28)

4,4,5,5-tetramethyl-2-[2-methyl-4-(1-methylcyclobutyl)phenyl]-1,3,2-dioxaborolane (Intermediate B-28) was prepared from 2-methyl-4-(1-methylcyclobutyl)phenol using a procedure analogous to Intermediate B-26 (Step 4 and Step 5) as a white solid. ¹H NMR (400 MHz, CDCl₃) δ (ppm) 7.73 (d, J=8.3 Hz, 1H), 7.00-6.95 (m, 2H), 2.54 (s, 3H), 2.42-2.34 (m, 2H), 2.16-2.00 (m, 3H), 1.86-1.76 (m, 1H), 1.44 (s, 3H), 1.33 (s, 12H). GCMS m/z calc. 286.21, found 286.20 (M).

### Intermediate B-29

### 2-[4-(1,1-dimethylpropyl)-2-methyl-phenyl]-4,4,5,5-tetramethyl-1,3,2-dioxaborolane

### Step 1: 2-[4-(1,1-dimethylpropyl)-2-methyl-phenyl]-4,4,5,5-tetramethyl-1,3,2-dioxaborolane (Intermediate B-29)

2-[4-(1,1-dimethylpropyl)-2-methyl-phenyl]-4,4,5,5-tetramethyl-1,3,2-dioxaborolane (Intermediate B-29) was prepared from 2-methyl-4-(tert-pentyl)phenol using procedure analogous to that found in Intermediate B-26 (Step 4 and Step 5) by using microwave irradiation at 120° C. for 1 h. NMR (400 MHz, DMSO-d₆) δ (ppm) 7.64-7.45 (m, 1H), 7.11 (d, J=7.5 Hz, 2H), 2.45 (s, 3H), 1.60 (q, J=7.4 Hz, 2H), 1.28 (s, 12H), 1.21 (s, 6H), 0.60 (t, J=7.4 Hz, 3H).

### Intermediate B-30

### 2-(4-isopropyl-2,5-dimethyl-phenyl)-4,4,5,5-tetramethyl-1,3,2-dioxaborolane

### Step 1: 4-isopropyl-2,5-dimethyl-phenol

4-bromo-2,5-dimethyl-phenol (1.35 g, 6.71 mmol), 2-isopropenyl-4,4,5,5-tetramethyl-1,3,2-dioxaborolane (1.68 g, 10.02 mmol), Pd(dppf)₂Cl₂·DCM (502 mg, 0.61 mmol), and aqueous potassium carbonate (7 mL of 2 M, 14 mmol) were combined in dioxane (25 mL) and heated at 80° C. for 16 h. The reaction was filtered and partitioned between ethyl acetate and 1 M HCl. The organics were separated, washed with brine, dried over sodium sulfate and evaporated. The crude material was dissolved in methanol (20 mL) and Pd/C wet (732 mg of 5% w/w, 0.34 mmol) was added. The reaction was stirred under a balloon of hydrogen for 3 h. The reaction was filtered through celite and washed with methanol. The solvent was evaporated and purification by silica gel chromatography eluting with 0-30% ethyl acetate in hexanes gave 4-isopropyl-2,5-dimethyl-phenol (406 mg, 37%). ¹H NMR (400 MHz, CDCl₃) δ (ppm) 6.96 (s, 1H), 6.56 (s, 1H), 4.40 (s, 1H), 3.03 (hept, J=6.9 Hz, 1H), 2.25 (s, 3H), 2.21 (s, 3H), 1.19 (d, J=6.9 Hz, 6H).

### Step 2: 2-(4-isopropyl-2,5-dimethyl-phenyl)-4,4,5,5-tetramethyl-1,3,2-dioxaborolane (Intermediate B-30)

2-(4-isopropyl-2,5-dimethyl-phenyl)-4,4,5,5-tetramethyl-1,3,2-dioxaborolane (Intermediate B-30) was prepared from 4-isopropyl-2,5-dimethylphenol using procedure analogous to that found in Intermediate B-24, Step 1. ¹H NMR (400 MHz, CDCl₃) δ (ppm) 7.54 (s, 1H), 7.04 (s, 1H), 3.10 (p, J=6.8 Hz, 1H), 2.50 (s, 3H), 2.29 (s, 3H), 1.32 (s, 12H), 1.21 (d, J=6.8 Hz, 6H).

### Intermediate B-31

### 2-(5-fluoro-4-isopropyl-2-methyl-phenyl)-4,4,5,5-tetramethyl-1,3,2-dioxaborolane

### Step 1: 2-(5-fluoro-4-isopropyl-2-methyl-phenyl)-4,4,5,5-tetramethyl-1,3,2-dioxaborolane (Intermediate B-31)

2-(5-fluoro-4-isopropyl-2-methyl-phenyl)-4,4,5,5-tetramethyl-1,3,2-dioxaborolane (Intermediate B-31) was prepared from 4-bromo-5-fluoro-2-methylphenol using procedure analogous to that found in Intermediate B-30, Step1, followed by a procedure analogous to that found in Intermediate B-24, Step 1 starting with 5-fluoro-4-isopropyl-2-methyl-phenol. ¹H NMR (400 MHz, CDCl₃) δ (ppm) 7.37 (d, J=11.0 Hz, 1H), 7.01 (d, J=7.1 Hz, 1H), 3.19 (hept, J=6.9 Hz, 1H), 2.48 (s, 3H), 1.32 (s, 12H), 1.23 (d, J=6.9 Hz, 6H). ¹⁹F NMR (376 MHz, CDCl₃) δ (ppm) −126.07 (dd, J(H−F)=11.2, 7.2 Hz, 1F).

### Intermediate B-32

### 2-(4-tert-butyl-5-cyclopropyl-2-methyl-phenyl)-4,4,5,5-tetramethyl-1,3,2-dioxaborolane

### Step 1: 5-bromo-4-tert-butyl-2-methyl-phenol

To a solution of 5-bromo-2-methyl-phenol (1 g, 5.35 mmol) and 2-methylpropan-2-ol (1.6 mL, 16.73 mmol) in heptane (5 mL) cooled to 0° C. was added sulfuric acid (570 μL, 10.69 mmol) and the reaction mixture was stirred at room temperature for 16 h. The reaction mixture was quenched with water and ethyl acetate was added. The layers were separated, and the aqueous layer was extracted with ethyl acetate (2×). The combined organic layer was dried over magnesium sulfate, filtered and concentrated. Purification via silica gel column chromatography using 0 to 5% ethyl acetate in hexanes gave 5-bromo-4-tert-butyl-2-methyl-phenol (760 mg, 58%). ESI-MS m/z calc. 242.03, found 243.0 (M+1)⁺. ¹H NMR (400 MHz, DMSO-d₆) δ (ppm) 9.51 (s, 1H), 7.14 (s, 1H), 7.01 (s, 1H), 2.07 (s, 3H), 1.41 (s, 9H).

### Step 2: 4-tert-butyl-5-cyclopropyl-2-methyl-phenol

A mixture of 5-bromo-4-tert-butyl-2-methyl-phenol (300 mg, 1.234 mmol), cyclopropylboronic acid (165 mg, 1.921 mmol Pd(dppf)Cl₂ (70 mg, 0.1278 mmol) and potassium carbonate (350 mg, 2.532 mmol) in dioxane (3 mL) was degassed under nitrogen, sealed and was heated at 100° C. for 16 h. The reaction was diluted with ethyl acetate and washed with water. The organic phase was dried over sodium sulfate, filtered and evaporated to dryness. The crude compound was purified via silica gel column chromatography using 0 to 5% ethyl acetate in hexanes to obtain 4-tert-butyl-5-cyclopropyl-2-methyl-phenol (165 mg, 65%). ESI-MS m/z calc. 204.15, found 205.08 (M+1)⁺. ¹H NMR (400 MHz, CDCl₃) δ (ppm) 7.10 (s, 1H), 6.30 (s, 1H), 2.26 (td, J=9.0, 3.9 Hz, 1H), 2.20 (s, 3H), 1.47 (s, 9H), 0.97 (dt, J=8.5, 3.1 Hz, 2H), 0.77-0.69 (m, 2H).

### Step 3: 2-(4-tert-butyl-5-cyclopropyl-2-methyl-phenyl)-4,4,5,5-tetramethyl-1,3,2-dioxaborolane (Intermediate B-32)

2-(4-tert-butyl-5-cyclopropyl-2-methyl-phenyl)-4,4,5,5-tetramethyl-1,3,2-dioxaborolane (Intermediate B-32) was prepared from 4-tert-butyl-5-cyclopropyl-2-methyl-phenol using procedure analogous to that found in Intermediate B-24, Step 1. ESI-MS m/z calc. 314.2417, found 315.3 (M+1)⁺. H NMR (400 MHz, CDCl₃) δ (ppm) 7.31 (s, 1H), 7.18 (s, 1H), 2.49 (s, 3H), 2.27-2.21 (m, 1H), 1.49 (s, 9H), 1.30 (s, 12H), 1.00-0.92 (m, 2H), 0.88-0.83 (m, 2H).

### Intermediate B-33

### 2-(4-tert-butyl-5-isopropyl-2-methyl-phenyl)-4,4,5,5-tetramethyl-1,3,2-dioxaborolane

### Step 1: 1-benzyloxy-5-bromo-4-tert-butyl-2-methyl-benzene

A solution of 5-bromo-4-tert-butyl-2-methyl-phenol (Intermediate B-32, Step 1, 658 mg, 2.706 mmol), benzyl bromide (355 μL, 2.99 mmol) and potassium carbonate (450 mg, 3.26 mmol) in ACN (7 mL) was heated at reflux for 16 h. The reaction mixture was quenched with water and the aqueous layer was extracted with DCM (3×). The combined organic layer was dried over magnesium sulfate, filtered and concentrated. Purification via silica gel column chromatography using hexanes gave 1-benzyloxy-5-bromo-4-tert-butyl-2-methyl-benzene (750 mg, 83%)¹H-NMR (400 MHz, DMSO-d₆) δ (ppm) 7.48-7.37 (m, 4H), 7.36-7.30 (m, 1H), 7.25 (d, J=0.9 Hz, 1H), 7.20 (s, 1H), 5.12 (s, 2H), 2.15 (s, 3H), 1.43 (s, 9H).

### Step 2: 4-tert-butyl-5-isopropyl-2-methyl-phenol

Under an atmosphere of nitrogen, to a microwave vial charged with 1-benzyloxy-5-bromo-4-tert-butyl-2-methyl-benzene (235 mg, 0.71 mmol) in dioxane (2.5 mL) was added Pd(dppf)Cl₂ (40 mg, 0.07 mmol), 2-isopropenyl-4,4,5,5-tetramethyl-1,3,2-dioxaborolane (240 mg, 1.43 mmol) and sodium carbonate (1 mL of 2M, 2 mmol). The reaction mixture was sealed and heated at 100° C. for 3 h. The reaction mixture was filtered, concentrated and purified via silica gel column chromatography using 0 to 5% ethyl acetate in hexanes to obtain 1-benzyloxy-4-tert-butyl-5-isopropenyl-2-methyl-benzene (139 mg, 67%), which was taken up in THF (2 mL) and Pd/C (wet) (100 mg, 0.1 mmol) was added and it was degassed under vacuum and a balloon filled with hydrogen was placed on it. It was stirred for 24 h under an atmosphere of hydrogen, filtered and the solvent was evaporated to obtain 4-tert-butyl-5-isopropyl-2-methyl-phenol (80 mg, 55%). ESI-MS m/z calc. 206.17, found 206.73 (M+1)⁺. ¹H NMR (400 MHz, DMSO-d₆) δ (ppm) 8.81 (s, 1H), 6.90 (s, 1H), 6.68 (s, 1H), 3.44 (p, J=6.8 Hz, 1H), 2.04 (s, 3H), 1.31 (s, 9H), 1.15 (d, J=6.7 Hz, 6H).

### Step 3: 2-(4-tert-butyl-5-isopropyl-2-methyl-phenyl)-4,4,5,5-tetramethyl-1,3,2-dioxaborolane (Intermediate B-33)

2-(4-tert-butyl-5-isopropyl-2-methyl-phenyl)-4,4,5,5-tetramethyl-1,3,2-dioxaborolane (Intermediate B-33) was prepared from 4-tert-butyl-5-isopropyl-2-methyl-phenol using procedure analogous to that found in Intermediate B-24, Step 1. ESI-MS m/z calc. 316.26, found 317.3 (M+1)⁺.

### Intermediate B-34

### 2-(4-tert-butyl-2-fluoro-6-methyl-phenyl)-4,4,5,5-tetramethyl-1,3,2-dioxaborolane

### Step 1: 4-tert-butyl-2-fluoro-6-methyl-phenol

To a stirring suspension of aluminium chloride (32 mg, 0.24 mmol) in 2-fluoro-6-methyl-phenol (200 mg, 0.16 mL, 1.59 mmol) was added 2-chloro-2-methyl-propane (255 mg, 0.3 mL, 2.76 mmol). The reaction mixture was stirred at room temperature for 18 h. The reaction mixture was poured onto water (10 mL) and ethyl acetate (10 mL) was added. The layers were separated, and the organic layer was washed with brine (10 mL), dried over magnesium sulfate, filtered and concentrated to give 4-tert-butyl-2-fluoro-6-methyl-phenol (183 mg, 63%) as a purple oil. ¹H-NMR (400 MHz, CDCl₃) δ (ppm) 6.96-6.91 (m, 2H), 2.28 (s, 3H), 1.28 (s, 9H). ¹⁹F-NMR (376 MHz, CDCl₃) δ (ppm) −142.1 (d, J=12.3 Hz, 1F).

### Step 2: (4-tert-butyl-2-fluoro-6-methyl-phenyl) trifluoromethanesulfonate

To a solution of 4-tert-butyl-2-fluoro-6-methyl-phenol (4.8 g, 24.97 mmol) in DCM (100 mL) containing pyridine (3.91 g, 4 mL, 49.46 mmol) at 0° C. was added triflic anhydride (8.38 g, 5 mL, 29.72 mmol) dropwise. The resulting mixture was warmed to room temperature and stirred overnight. The reaction was quenched by careful addition of saturated sodium hydrogen carbonate (50 mL). The layers were separated, and the organic was washed successively with 0.5 N HCl (50 mL), saturated sodium hydrogen carbonate (50 mL), water (50 mL), brine (50 mL), dried over sodium sulfate and concentrated. The crude was purified by silica gel column chromatography using 5 to 20% ethyl acetate in heptanes to give (4-tert-butyl-2-fluoro-6-methyl-phenyl) trifluoromethanesulfonate (7.44 g, 91%) a clear oil. ESI-MS m/z calc. 314.06, found 312.86 (M−1)⁻; ¹H-NMR (400 MHz, CDCl₃) δ (ppm) 7.11-7.08 (m, 2H), 2.42 (s, 3H), 1.33 (s, 9H).

### Step 3: 2-(4-tert-butyl-2-fluoro-6-methyl-phenyl)-4,4,5,5-tetramethyl-1,3,2-dioxaborolane (Intermediate B-34)

A mixture of (4-tert-butyl-2-fluoro-6-methyl-phenyl) trifluoromethanesulfonate (500 mg, 1.51 mmol), bis(pinacolato)diboron (420 mg, 1.65 mmol), potassium acetate (370 mg, 3.77 mmol), XPhos (144 mg, 0.30 mmol), palladium acetate (34 mg, 0.15 mmol) and lithium chloride (13 mg, 0.31 mmol) in dioxane (4 mL) was stirred at 100° C. under an atmosphere of argon for 2 h. The reaction mixture was cooled to room temperature and diluted with ethyl acetate (20 mL). The organic layer was washed with water (20 mL), brine (20 mL), dried over magnesium sulfate and concentrated. Purification of the crude by reverse phase chromatography using 20 to 95% acetonitrile in water (0.1% formic acid) gave 2-(4-tert-butyl-2-fluoro-6-methyl-phenyl)-4,4,5,5-tetramethyl-1,3,2-dioxaborolane (Intermediate B-34, 68 mg, 15%). ¹H-NMR (400 MHz, CDCl₃) δ (ppm) 6.95 (d, J=0.9 Hz, 1H), 6.84 (dd, J=11.4, 0.9 Hz, 1H), 2.46 (s, 3H), 1.37 (s, 12H), 1.28 (s, 9H). 19F-NMR (376 MHz, CDCl₃) δ (ppm) −104.4 (d, J=11.3 Hz, 1F).

### Intermediate B-35

### 2-(5-isopropyl-2,4-dimethyl-phenyl)-4,4,5,5-tetramethyl-1,3,2-dioxaborolane

### Step 1: 5-isopropenyl-2,4-dimethyl-phenol

To a mixture of 5-bromo-2,4-dimethyl-phenol (200 mg, 0.83 mmol), potassium isopropenyltrifluoroborate (156 mg, 1.05 mmol) and potassium carbonate (422 mg, 3.05 mmol) in 1,4-dioxane (4 mL) and water (1 mL) was added Pd(dppf)Cl₂ (64 mg, 0.09 mmol). The reaction mixture was subjected to microwave irradiation at 120° C. for 1 hour. The reaction mixture was partitioned between ethyl acetate (30 mL) and water (10 mL). The organic phase was washed with brine (20 mL), dried over sodium sulfate and concentrated in vacuo to give 5-isopropenyl-2,4-dimethyl-phenol (214 mg, 99%). ESI-MS m/z calc. 162.10, found 161.0 (M−1)⁻. ¹H-NMR (400 MHz, CDCl₃) δ (ppm) 6.91 (s, 1H), 6.56 (s, 1H), 5.14 (t, J=1.8 Hz, 1H), 4.81 (q, J=1.1 Hz, 1H), 4.72 (s, 1H), 2.20 (s, 6H), 1.99 (s, 3H)

### Step 2: 5-isopropyl-2,4-dimethyl-phenol

A solution of 5-isopropenyl-2,4-dimethyl-phenol (1.63 g, 7.92 mmol) in methanol (20 mL) containing Pd/C (84 mg, 10% w/w, 0.08 mmol) was stirred under an atmosphere of hydrogen for 4 h. The catalyst was removed by filtration and washed with methanol (20 mL). The filtrate was concentrated in vacuo and purified by silica gel column chromatography using 2 to 5% ethyl acetate in heptane to afford 5-isopropyl-2,4-dimethyl-phenol (1.41 g, 93%) as a yellow oil. ESI-MS m/z calc. 164.12, found 163.1 (M−1)⁻. ¹H-NMR (400 MHz, CDCl₃) δ (ppm) 6.88 (s, 1H), 6.67 (s, 1H), 4.51 (br s, 1H), 3.08-2.99 (m, 1H), 2.22 (s, 3H), 2.19 (s, 3H), 1.19 (d, J=6.9 Hz, 6H).

### Step 3: 2-(5-isopropyl-2,4-dimethyl-phenyl)-4,4,5,5-tetramethyl-1,3,2-dioxaborolane (Intermediate B-35)

2-(5-isopropyl-2,4-dimethyl-phenyl)-4,4,5,5-tetramethyl-1,3,2-dioxaborolane (Intermediate B-35, 336 mg, 98%) was prepared from 5-isopropyl-2,4-dimethyl-phenol using procedure analogous to that found in Intermediate B-26 (Step 4 and Step 5). ¹H-NMR (400 MHz, CDCl₃) δ (ppm) 7.62 (s, 1H), 6.94 (s, 1H), 3.15-3.05 (m, 1H), 2.46 (s, 3H), 2.30 (s, 3H), 1.32 (s, 12H), 1.24 (d, J=6.9 Hz, 6H).

### Intermediate B-36

### 4,4,5,5-tetramethyl-2-(3,3,7-trimethyltetralin-6-yl)-1,3,2-dioxaborolane

### Step 1: 7-methoxy-2,2,6-trimethyl-tetralin-1-one

To a stirred solution of 7-methoxy-6-methyl-tetralin-1-one (200 mg, 1.05 mmol) in dry THF (5 mL) was added sodium hydride in mineral oil (210 mg, 60% w/w, 5.25 mmol). After stirring the mixture for 1 h at 0° C., iodomethane (684 mg, 0.3 mL, 4.82 mmol) in dry THF (0.5 mL) was added slowly. The mixture was warmed to room temperature and stirred overnight. The reaction mixture was quenched by slow dropwise addition of water. The aqueous layer was extracted with ethyl acetate (2×10 mL), washed with water (5 mL), brine (5 mL), dried over sodium sulfate, filtered and concentrated to give 7-methoxy-2,2,6-trimethyl-tetralin-1-one (260 mg, 100%). ESI-MS m/z calc. 218.13, found 219.0 (M+1)⁺. ¹H-NMR (400 MHz, CDCl₃) δ (ppm) 7.44 (s, 1H), 6.97 (s, 1H), 3.85 (s, 3H), 2.87 (t, J=6.2 Hz, 2H), 2.23 (s, 3H), 1.94 (t, J=6.2 Hz, 2H), 1.19 (s, 6H).

### Step 2: 7-methoxy-2,2,6-trimethyl-tetralin

To a solution of 7-methoxy-2,2,6-trimethyl-tetralin-1-one (4.15 g, 18.53 mmol) in TFA (70 mL) was added Et₃SiH (13.1 g, 18 mL, 112.7 mmol) dropwise and the resulting mixture was stirred at room temperature overnight. The reaction mixture was concentrated under reduced pressure and the residue was partitioned between water (20 mL) and ethyl acetate (100 mL). The layers were separated, and the organic layer was washed with saturated sodium bicarbonate solution (50 mL) and brine (40 mL), dried over sodium sulfate and concentrated to give 7-methoxy-2,2,6-trimethyl-tetralin (9 g, 100%). ESI-MS m/z calc. 204.15, found 205.08 (M+1)⁺. ¹H-NMR (400 MHz, CDCl₃) δ (ppm) 6.85 (s, 1H), 6.48 (s, 1H), 3.78 (s, 3H), 2.68 (t, J=6.6 Hz, 2H), 2.48 (s, 2H), 2.15 (s, 3H), 1.51 (t, 6.6 Hz, 2H), 0.97 (s, 6H).

### Step 3: 3,3,7-trimethyltetralin-6-ol

Boron tribromide in DCM (37 mL of 1 M, 37 mmol) was added dropwise to a solution 7-methoxy-2,2,6-trimethyl-tetralin (9 g, 18.5 mmol) in DCM (100 mL) at 0° C. under an atmosphere of argon. The reaction mixture was warmed to room temperature and stirred for 2 hours. The mixture was then poured portion-wise onto ice-water (200 mL) and stirred for 30 minutes. The layers were separated, and the aqueous layer was extracted with DCM (2×50 mL). The combined organic layer was washed with brine (50 mL), dried over sodium sulfate, filtered and concentrated. The crude material was purified by silica gel flash chromatography using 0 to 30% ethyl acetate in heptane to give 3,3,7-trimethyltetralin-6-ol (2.5 g, 70%). ESI-MS m/z calc. 190.13, found 189.0 (M−1)⁻. ¹H-NMR (400 MHz, CDCl₃) δ (ppm) 6.84 (s, 1H), 6.45 (s, 1H), 4.44 (br s, 1H), 2.68 (t, J=6.6 Hz, 2H), 2.43 (s, 2H), 2.19 (s, 3H), 1.51 (t, J=6.6 Hz, 2H), 0.96 (s, 6H).

### Step 2: 4,4,5,5-tetramethyl-2-(3,3,7-trimethyltetralin-6-yl)-1,3,2-dioxaborolane (Intermediate B-36)

4,4,5,5-tetramethyl-2-(3,3,7-trimethyltetralin-6-yl)-1,3,2-dioxaborolane (Intermediate B-36) was prepared from 3,3,7-trimethyltetralin-6-ol using procedure analogous to that found in Intermediate B-26 (Step 4 and Step 5) as a pale-yellow solid. ¹H-NMR (400 MHz, CDCl₃) δ (ppm) 7.45 (s, 1H), 6.90 (s, 1H), 2.75 (t, J=6.6 Hz, 2H), 2.49 (s, 2H), 2.46 (s, 3H), 1.53 (t, J=6.6 Hz, 2H), 1.32 (s, 12H), 0.95 (s, 6H).

### Intermediate B-37

### 2-[4-isopropyl-2-methyl-5-(trifluoromethyl)phenyl]-4,4,5,5-tetramethyl-1,3,2-dioxaborolane

### Step 1: 2-benzyloxy-1-methyl-4-(trifluoromethyl)benzene

A solution of 2-methyl-5-(trifluoromethyl)phenol (2 g, 11.35 mmol) in DMF (15 mL) was treated with sodium hydride (685 mg of 60% w/w, 17.13 mmol) at 0° C. Benzyl bromide (1700 μL, 14.29 mmol) was added and the reaction mixture was stirred at room temperature for 2 h. The reaction mixture was then quenched with aqueous ammonium chloride and the aqueous layer was extracted with DCM. The organic layer was washed with brine, dried over sodium sulfate, filtered and concentrated. Purification by silica gel chromatography using 0 to 10% ethyl acetate in hexanes gave 2-benzyloxy-1-methyl-4-(trifluoromethyl)benzene (2.65 g, 88%). ¹H-NMR (400 MHz, CDCl₃) δ (ppm) 7.48-7.38 (m, 4H), 7.33-7.37 (m, 1H), 7.26 (d, J=7.7 Hz, 1H), 7.15 (d, J=7.7 Hz, 1H), 7.11 (s, 1H), 5.11 (s, 2H), 2.32 (s, 3H). ¹⁹F NMR (376 MHz, CDCl₃) δ (ppm) −62.20.

### Step 2: 1-benzyloxy-4-bromo-2-methyl-5-(trifluoromethyl)benzene

A solution of 2-benzyloxy-1-methyl-4-(trifluoromethyl)benzene (2.4 g, 9.01 mmol) and sodium acetate (925 mg, 11.27 mmol) in acetic acid (2 mL) was treated with molecular bromine (1.80 g, 580 μL, 11.29 mmol) at room temperature. The reaction mixture was stirred at room temperature overnight and quenched with water and aqueous sodium thiosulfate. The aqueous layer was extracted with DCM and the organic phase was dried over sodium sulfate and concentrated. Purification by silica gel chromatography using hexanes gave 1-benzyloxy-4-bromo-2-methyl-5-(trifluoromethyl)benzene (2 g, 25%). ¹H NMR (400 MHz, CDCl₃) δ (ppm) 7.49-7.33 (m, 6H), 7.18 (s, 1H), 5.10 (s, 2H), 2.29 (s, 3H). ¹⁹F NMR (377 MHz, CDCl₃) δ (ppm) −62.21 (s, 3F).

### Step 3: 4-isopropyl-2-methyl-5-(trifluoromethyl)phenol

A 20 mL microwave vial charged with 1-benzyloxy-4-bromo-2-methyl-5-(trifluoromethyl)benzene (2 g, 3.59 mmol), CPhos (101 mg, 0.23 mmol), Pd(OAc)₂ (26 mg, 0.12 mmol) was evacuated/backfilled with nitrogen. THF (10 mL) was added followed by bromo(isopropyl)zinc (18 mL of 0.5 M, 9 mmol) at room temperature. The reaction mixture was stirred at room temperature overnight and quenched with a solution of ammonium chloride. The aqueous layer was extracted with ethyl acetate and was dried over sodium sulfate, filtered and concentrated. Purification by silica gel column chromatography using hexanes gave 1-benzyloxy-4-isopropyl-2-methyl-5-(trifluoromethyl)benzene (1.3 g, 59%). The obtained intermediate was dissolved in methanol (10 mL) and Pd/C (430 mg of 10% w/w, 0.40 mmol) was added. The mixture was sparged with hydrogen from a balloon while stirring at room temperature for 2 h. The reaction mixture was filtered and purified by silica gel chromatography using 0 to 5% ethyl acetate in hexanes to afford 4-isopropyl-2-methyl-5-(trifluoromethyl)phenol (800 mg, 43%). ¹H NMR (400 MHz, CDCl₃) δ (ppm) 7.18 (s, 1H), 6.98 (s, 1H), 4.98 (s, 1H), 3.31-3.17 (m, 1H), 2.28 (s, 3H), 1.22 (d, J=6.8 Hz, 6H).

### Step 4: 2-[4-isopropyl-2-methyl-5-(trifluoromethyl)phenyl]-4,4,5,5-tetramethyl-1,3,2-dioxaborolane (Intermediate B-37)

2-[4-isopropyl-2-methyl-5-(trifluoromethyl)phenyl]-4,4,5,5-tetramethyl-1,3,2-dioxaborolane (Intermediate B-37) was prepared from 4-isopropyl-2-methyl-5-(trifluoromethyl)phenol using procedure analogous to that found in Intermediate B-26 (Step 4 and Step 5). ESI-MS m/z calc. 328.18, found 329.3 (M+1)⁺. ¹H NMR (400 MHz, CDCl₃) δ (ppm) 7.97 (s, 1H), 7.23 (s, 1H), 3.31 (hept, J=6.7 Hz, 1H), 2.56 (s, 3H), 1.34 (s, 12H), 1.24 (d, J=6.7 Hz, 6H).

### Intermediate B-38

### 2-(4-tert-butyl-2-fluoro-phenyl)-4,4,5,5-tetramethyl-1,3,2-dioxaborolane

### Step 1: 2-(4-tert-butyl-2-fluoro-phenyl)-4,4,5,5-tetramethyl-1,3,2-dioxaborolane (Intermediate B-38)

2-(4-tert-butyl-2-fluoro-phenyl)-4,4,5,5-tetramethyl-1,3,2-dioxaborolane (Intermediate B-38, 1.46 g, 95%) was prepared from 4-tert-butyl-2-fluoro-phenol using procedure analogous to that found in Intermediate B-26 (Step 4 and Step 5). ¹H-NMR (400 MHz, CDCl₃) δ (ppm) 7.67 (t, J=7.1 Hz, 1H), 7.18-7.15 (m, 1H), 7.05 (dd, J=11.7, 1.6 Hz, 1H), 1.36 (s, 12H), 1.31 (s, 9H). ¹⁹F-NMR (376 MHz, CDCl₃) δ (ppm) −102.9 (dd, J=11.6, 6.8 Hz, 1F).

### Intermediate B-39

### 4,4,5,5-tetramethyl-2-(2,2,7-trimethyltetralin-6-yl)-1,3,2-dioxaborolane

### Step 1: 6-methoxy-2,2,7-trimethyl-tetralin-1-one

To a solution of 6-methoxy-7-methyl-tetralin-1-one (1 g, 5.26 mmol) and iodomethane (3.88 g, 1.7 mL, 27.31 mmol) in THF (14 mL) at 0° C. was added portion wise sodium hydride in mineral oil (841 mg, 60% w/w, 21.03 mmol). The reaction mixture was stirred at 0° C. for 2 h and then stirred at room temperature overnight. It was cooled to 0° C. and water (10 mL) was carefully added. The residue was diluted with a 1 M aqueous solution of HCl (60 mL). The layers were separated, and the aqueous layer was extracted with ethyl acetate (2×80 mL). The combined organic layer was combined and washed with brine (40 mL), dried over anhydrous sodium sulfate, filtered and concentrated under reduced pressure. The crude product was purified by silica gel column chromatography using 0 to 20% ethyl acetate in heptanes to afford 6-methoxy-2,2,7-trimethyl-tetralin-1-one (952 mg, 83%) as an off-white solid. ESI-MS m/z calc. 218.13, found 219.2 (M+1)⁺. ¹H NMR (400 MHz, CDCl₃) δ (ppm) 7.83 (s, 1H), 6.58 (s, 1H), 3.88 (s, 3H), 2.94 (t, J=6.4 Hz, 2H), 2.21 (s, 3H), 1.97 (t, J=6.4 Hz, 2H), 1.21 (s, 6H).

### Step 2: 6-methoxy-2,2,7-trimethyl-tetralin

A solution of 6-methoxy-2,2,7-trimethyl-tetralin-1-one (952 mg, 4.36 mmol) in methanol (7 mL) was added to a mixture of palladium (II) chloride (70 mg, 0.39 mol) and poly(methylhydrosiloxane) (2.11 g, 2.1 mL, 35.14 mmol) in methanol (14 mL). The reaction mixture was stirred at 80° C. for 2.5 h. A second portion of poly(methylhydrosiloxane) (2.11 g, 2.1 mL, 35.14 mmol) was added to the mixture at room temperature and it was stirred at 80° C. for 18 h. A third portion of poly(methylhydrosiloxane) (2.11 g, 2.1 mL, 35.14 mmol) was added to the mixture at room temperature and it was stirred at 80° C. for 4 h. It was cooled to room temperature and concentrated under reduced pressure. The residue was dissolved in anhydrous methanol (20 mL), then palladium (II) chloride (70 mg, 0.39 mmol) and a fourth portion of poly(methylhydrosiloxane) (8 g, 8 mL, 133.84 mmol) were added to the mixture at room temperature and it was stirred at 80° C. for 48 h. The solvent was evaporated under reduced pressure. The crude product was purified by silica gel flash chromatography using 0 to 5% ethyl acetate in heptanes to afford 6-methoxy-2,2,7-trimethyl-tetralin (538 mg, 60%) as a light-yellow oil. ¹H NMR (400 MHz, CDCl₃) δ (ppm) 6.80 (s, 1H), 6.56 (s, 1H), 3.80 (s, 3H), 2.77 (t, J=6.7 Hz, 2H), 2.44 (s, 2H), 2.17 (s, 3H), 1.54 (t, J=6.7 Hz, 2H, overlapped with water), 0.98 (s, 6H).

### Step 3: 2,2,7-trimethyltetralin-6-ol

To a solution of 6-methoxy-2,2,7-trimethyl-tetralin (538 mg, 2.62 mmol) in anhydrous DCM (35 mL) at −70° C. was added dropwise a solution of boron tribromide (1.3 g, 0.5 mL, 5.19 mmol) in DCM (5 mL). The mixture was then stirred at −60° C. for 2 h and then stirred at room temperature for 2.5 h. The reaction was quenched with methanol (20 mL), diluted with a saturated solution of sodium bicarbonate (40 mL) and stirred at room temperature overnight. The layers were separated, and the aqueous layer was extracted with DCM (3×100 mL). The combined organic layer was washed with brine (40 ml), dried over sodium sulfate, filtered and concentrated under reduced pressure to afford crude 2,2,7-trimethyltetralin-6-ol (500 mg, 100%). ESI-MS m/z calc. 190.14, found 191.2 (M+1)⁺. ¹H NMR (400 MHz, DMSO-d₆) δ (ppm) 8.81 (s, 1H), 6.67 (s, 1H), 6.46 (s, 1H), 2.59 (t, J=6.6 Hz, 2H), 2.32 (s, 2H), 2.02 (s, 3H), 1.44 (t, J=6.7 Hz, 2H), 0.90 (s, 6H).

### Step 4: 4,4,5,5-tetramethyl-2-(2,2,7-trimethyltetralin-6-yl)-1,3,2-dioxaborolane (Intermediate B-39)

4,4,5,5-tetramethyl-2-(2,2,7-trimethyltetralin-6-yl)-1,3,2-dioxaborolane (Intermediate B-39) was prepared from 2,2,7-trimethyltetralin-6-ol using procedure analogous to that found in Intermediate B-26 (Step 4 and Step 5). ESI-MS m/z calc. 300.22, found 301.2 (M+1)⁺. ¹H NMR (400 MHz, CDCl₃) δ (ppm) 7.52 (s, 1H), 6.85 (s, 1H), 2.77 (t, J=6.7 Hz, 2H), 2.49 (s, 2H), 2.47 (s, 3H), 1.57-1.53 (m, 2H, overlapped with water), 1.34 (s, 12H), 0.97 (s, 6H).

### Intermediate B-40

### 4,4,5,5-tetramethyl-2-(2,2,6-trimethylindan-5-yl)-1,3,2-dioxaborolane

### Step 1: 5-methoxy-2,2-dimethyl-indan-1-one

Sodium hydride (60% in mineral oil) (5 g, 125.01 mmol) was slowly added at 0° C. to a solution of 5-methoxyindan-1-one (5 g, 30.83 mmol) and iodomethane (22.8 g, 10 mL, 160.63 mmol) in tetrahydrofuran (70 mL). The reaction mixture was stirred at room temperature for 3 h. The reaction mixture was cooled to 0° C. and carefully quenched with water (10 mL). The solvent was removed under reduced pressure and residue was diluted with aqueous 1 M HCl (60 mL). The aqueous layer was extracted with ethyl acetate (2×80 mL). The combined organic layer was washed with water and brine in a 1:1 ratio (40 mL), dried over anhydrous sodium sulfate, filtered and evaporated under reduced pressure. The crude product was purified by silica gel column chromatography using 0 to 15% ethyl acetate in heptanes to give 5-methoxy-2,2-dimethyl-indan-1-one (5.45 g, 93%) as a light-yellow oil. ESI-MS m/z calc. 190.09, found 191.1 (M+1)⁺. ¹H NMR (400 MHz, CDCl₃) δ (ppm) 7.69 (d, J=8.5 Hz, 1H), 6.90 (dd, J=8.4, 2.0 Hz, 1H), 6.85 (br s, 1H), 3.87 (s, 3H), 2.94 (s, 2H), 1.22 (s, 6H).

### Step 2: 5-methoxy-2,2-dimethyl-indane

A solution of 5-methoxy-2,2-dimethyl-indan-1-one (5.45 g, 28.62 mmol) in methanol (40 mL) was added to a mixture of palladium(II) chloride (440 mg, 2.48 mol) and poly(methylhydrosiloxane) (14.08 g, 14 mL, 234.24 mmol) in methanol (80 mL). The reaction mixture was stirred at 80° C. for 5 h. The solvent was evaporated under reduced pressure. The residue was purified by silica gel chromatography with 0 to 15% ethyl acetate in heptanes to give 5-methoxy-2,2-dimethyl-indane (4.7 g, 93%) as a colorless oil. GCMS m/z calc. 176.12, found 176.10 (M), H NMR (400 MHz, CDCl₃) δ (ppm) 7.05 (d, J=8.0 Hz, 1H), 6.73 (br. s, 1H), 6.68 (dd, J=8.2, 2.3 Hz, 1H), 3.78 (s, 3H), 2.69 (s, 2H), 2.66 (s, 2H), 1.15 (s, 6H).

### Step 3: 5-bromo-6-methoxy-2,2-dimethyl-indane

To a solution of 5-methoxy-2,2-dimethyl-indane (1.26 g, 6.91 mmol) in acetonitrile (35 mL) was added NBS (1.35 g, 7.59 mmol). The reaction mixture was stirred at room temperature for 65 h. It was diluted with ethyl acetate (150 mL). The organic layer was washed with aqueous saturated sodium bicarbonate/10% aqueous Na2SO3 1/1 (80 mL), water (40 mL) and brine (40 mL), dried over anhydrous sodium sulfate, filtered and evaporated under reduced pressure. The crude product was purified by silica gel chromatography with 0 to 15% ethyl acetate in heptanes to give 5-bromo-6-methoxy-2,2-dimethyl-indane (1.17 g, 65%) as a light-yellow oil. ESI-MS m/z calc. 254.03, found 255.1 (M+1)⁺. ¹H NMR (400 MHz, CDCl₃) δ (ppm) 7.31 (s, 1H), 6.74 (s, 1H), 3.86 (s, 3H), 2.68-2.62 (m, 4H), 1.14 (s, 6H).

### Step 4: 5-methoxy-2,2,6-trimethyl-indane

To a solution of 5-bromo-6-methoxy-2,2-dimethyl-indane (1.17 g, 4.46 mmol) in dioxane (10 mL) was added methylboronic acid (536 mg, 8.95 mmol) and potassium carbonate (1.85 g, 13.39 mmol). The reaction mixture was purged with nitrogen and Pd(dppf)₂Cl₂·DCM (370 mg, 0.45 mmol) was added. The reaction mixture was stirred at 100° C. for 16 h. The reaction mixture was cooled to room temperature, diluted with ethyl acetate (25 ml), and filtered through celite. The filtrate was evaporated under pressure and was purified by silica gel column chromatography using 0 to 20% ethyl acetate in hexanes to obtain 5-methoxy-2,2,6-trimethyl-indane (0.66 g, 74%) as a yellow liquid. ¹H NMR (400 MHz, CDCl₃) δ (ppm) 6.93 (s, 1H), 6.68 (s, 1H), 3.80 (s, 3H), 2.69 (s, 2H), 2.64 (s, 2H), 2.19 (s, 3H), 1.15 (s, 6H).

### Step 5: 2,2,6-trimethylindan-5-ol

In a 100 mL round bottom flask, 5-methoxy-2,2,6-trimethyl-indane (0.66 g, 3.47 mmol), was dissolved in DCM (45 mL) and cooled to −78° C. To the reaction mixture, boron tribromide in DCM (6.5 mL of 1 M, 6.5 mmol) was added dropwise. The reaction mixture was gradually warmed to room temperature and stirred overnight. The reaction mixture was quenched with methanol (7 mL) followed by the addition of saturated sodium bicarbonate solution. (14 mL) and was stirred at room temperature for 1 h. The aqueous layer was extracted with DCM (2×25 ml). The combined organic layer was washed with brine (40 ml), dried over sodium sulfate, filtered, and evaporated to dryness. Purification by silica gel column chromatography using 5 to 25% ethyl acetate in hexanes gave 2,2,6-trimethylindan-5-ol (0.55 g, 88%) as a yellow solid. ¹H NMR (400 MHz, CDCl₃) δ (ppm) 6.93 (s, 1H), 6.63 (s, 1H), 4.46 (s, 1H), 2.66-2.59 (m, 4H), 2.23 (s, 3H), 1.15 (s, 6H).

### Step 6: 4,4,5,5-tetramethyl-2-(2,2,6-trimethylindan-5-yl)-1,3,2-dioxaborolane

4,4,5,5-tetramethyl-2-(2,2,6-trimethylindan-5-yl)-1,3,2-dioxaborolane (Intermediate B-40) was prepared from 2,2,6-trimethylindan-5-ol using procedure analogous to that found in Intermediate B-26 (Step 4 and Step 5) as an off-white solid. ESI-MS m/z calc. 286.21, found 287.2 (M+1)⁺. ¹H NMR (400 MHz, CDCl₃) δ (ppm) 7.57 (s, 1H), 6.98 (s, 1H), 2.69-2.65 (m, 4H), 2.50 (s, 3H), 1.33 (s, 12H), 1.12 (s, 6H).

### Intermediate B-41

### 2-(5-ethyl-2,4-dimethyl-phenyl)-4,4,5,5-tetramethyl-1,3,2-dioxaborolane

### Step 1: 2,4-dimethyl-5-vinyl-phenol

A mixture of 5-bromo-2,4-dimethyl-phenol (200 mg, 0.85 mmol), potassium vinyltrifluoroborate (137 mg, 1.02 mmol) and potassium carbonate (412 mg, 2.98 mmol) in 1,4-dioxane (4 mL) and water (1 mL) was degassed with argon for 10 minutes. Pd(dppf)Cl₂ (36 mg, 0.05 mmol) was added, and the reaction mixture was heated at 100° C. for 4 hours. The reaction mixture was partitioned between ethyl acetate (30 mL) and water (10 mL). The layers were separated, and the organic layer was washed with brine (20 mL), dried over sodium sulfate, filtered and concentrated. The crude material was purified silica gel by flash chromatography using 0 to 20% ethyl acetate in heptanes to give 2,4-dimethyl-5-vinyl-phenol (100 mg, 77%) as colourless oil. ESI-MS m/z calc. 148.09, found 146.97 (M−1)⁻. ¹H-NMR (400 MHz, CDCl₃) δ (ppm) 6.90 (s, 1H), 6.89 (s, 1H), 6.86-6.82 (m, 1H), 5.55 (dd, J=17.4, 1.4 Hz, 1H), 5.22 (dd, J=10.8, 1.1 Hz, 1H), 4.49 (br s, 1H), 2.25 (s, 3H), 2.21 (s, 3H).

### Step 2: 5-ethyl-2,4-dimethyl-phenol

A suspension of 2,4-dimethyl-5-vinyl-phenol (930 mg, 5.79 mmol) and palladium on carbon (200 mg, 10% w/w, 0.19 mmol) in methanol (50 mL) was stirred at room temperature under an atmosphere of hydrogen for 5 hours. The reaction mixture was filtered through a pad of celite, washed with methanol (10 mL) and concentrated. Purification by silica gel flash chromatography using 0 to 10% ethyl acetate in heptanes gave 5-ethyl-2,4-dimethyl-phenol (960 mg, 95%). ESI-MS m/z calc. 150.10, found 149.0 (M−1)⁻. ¹H-NMR (400 MHz, CDCl₃) δ (ppm) 6.87 (s, 1H), 6.59 (s, 1H), 4.47 (br s, 1H), 2.53 (q, J=7.5 Hz, 2H), 2.18 (s, 6H), 1.17 (t, 7.6 Hz, 3H)

### Step 3: 2-(5-ethyl-2,4-dimethyl-phenyl)-4,4,5,5-tetramethyl-1,3,2-dioxaborolane (Intermediate B-41)

2-(5-ethyl-2,4-dimethyl-phenyl)-4,4,5,5-tetramethyl-1,3,2-dioxaborolane (Intermediate B-41) was prepared from 5-ethyl-2,4-dimethyl-phenol using procedure analogous to that found in Intermediate B-26 (Step 4 and Step 5) as pale-yellow oil. ¹H-NMR (400 MHz, CDCl₃) δ (ppm) 7.53 (s, 1H), 6.94 (s, 1H), 2.59 (q, J=7.5 Hz, 2H), 2.46 (s, 3H), 2.27 (s, 3H), 1.32 (s, 12H), 1.17 (t, J=7.6 Hz, 3H).

### Intermediate B-42

### 2-(2-fluoro-4-isopropyl-5-methyl-phenyl)-4,4,5,5-tetramethyl-1,3,2-dioxaborolane

### Step 1: 2-fluoro-4-isopropyl-5-methyl-phenol

4-bromo-2-fluoro-5-methyl-phenol (937 mg, 4.57 mmol), 2-isopropenyl-4,4,5,5-tetramethyl-1,3,2-dioxaborolane (1.57 g, 9.33 mmol), Pd(dppf)Cl₂·DCM (353 mg, 0.43 mmol), and aqueous potassium carbonate (4.8 mL of 2 M, 9.6 mmol) were combined in dioxane (17 mL) and heated at 80° C. for 16 h. The reaction was filtered and partitioned between ethyl acetate and 1 M HCl. The organics were separated, washed with brine, dried over sodium sulfate and evaporated. The crude material was dissolved in methanol (15 mL) and Pd/C wet (500 mg of 5% w/w, 0.23 mmol) was added. The reaction was stirred under a balloon of hydrogen for 3 h. The reaction mixture was filtered through celite and washed with methanol. The solvent was evaporated and purification by silica gel chromatography eluting with 0-30% ethyl acetate in hexanes gave 2-fluoro-4-isopropyl-5-methyl-phenol (618 mg, 80%). ¹H NMR (400 MHz, CDCl₃) δ (ppm) 6.92 (d, J=12.4 Hz, 1H), 6.78-6.73 (m, 1H), 4.84 (d, J=3.8 Hz, 1H), 3.07-2.97 (m, 1H), 2.24 (s, 3H), 1.17 (d, J=6.8 Hz, 6H).

### Step 2: 2-(2-fluoro-4-isopropyl-5-methyl-phenyl)-4,4,5,5-tetramethyl-1,3,2-dioxaborolane (Intermediate B-42)

2-(2-fluoro-4-isopropyl-5-methyl-phenyl)-4,4,5,5-tetramethyl-1,3,2-dioxaborolane (Intermediate B-42) was prepared from 2-fluoro-4-isopropyl-5-methyl-phenol using procedure analogous to that found in Intermediate B-24 (Step 1). ¹H NMR (400 MHz, CDCl₃) δ (ppm) 7.48 (d, J=6.4 Hz, 1H), 6.91 (d, J=11.0 Hz, 1H), 3.15-3.03 (m, 1H), 2.28 (s, 3H), 1.35 (s, 12H), 1.20 (d, J=6.8 Hz, 6H). ¹⁹F NMR (376 MHz, CDCl₃) δ (ppm) −106.81 (dd, J=11.0, 6.5 Hz, 1F).

### Intermediate B-43

### 2-(4-tert-butyl-2-fluoro-3,6-dimethyl-phenyl)-4,4,5,5-tetramethyl-1,3,2-dioxaborolane

### Step 1: 2-bromo-4-tert-butyl-3,6-dimethyl-phenol

To a solution of 4-tert-butyl-2,5-dimethyl-phenol (10.1 g, 48.16 mmol) in acetonitrile (100 mL) was added NBS (11 g, 61.80 mmol). The reaction mixture was stirred at room temperature for 17 h. Additional NBS (8 g, 44.95 mmol) was added. The reaction mixture was stirred at rt for 6 h. The reaction mixture was poured onto a stirring mixture of saturated sodium bicarbonate (100 mL), sodium thiosulfate (100 mL) and MTBE (200 mL). The layers were separated and the organic layer was washed with brine (50 mL), dried over sodium sulfate, filtered and evaporated under reduced pressure. The residue was triturated with heptanes (30 mL). The solid was removed by filtration and washed with heptanes. The filtrate was concentrated under reduced pressure. The residue was purified by silica gel column chromatography using 0 to 10% ethyl acetate in heptanes to give 2-bromo-4-tert-butyl-3,6-dimethyl-phenol (12.3 g, 99%) as a light-yellow solid. GCMS m/z calc. 256.04, found 255.80 (M). ¹H NMR (400 MHz, CDCl₃) δ (ppm) 7.07-7.04 (m, 1H), 2.42 (s, 3H), 1.99 (d, J=1.3 Hz, 3H), 1.15 (s, 9H).

### Step 2: 3-bromo-1-tert-butyl-4-(methoxymethoxy)-2,5-dimethyl-benzene

To a solution of 2-bromo-4-tert-butyl-3,6-dimethyl-phenol (66 mg, 0.26 mmol) and DIPEA (103.88 mg, 0.14 mL, 0.80 mmol) in DCM (2 mL) was added chloromethyl methyl ether (53 mg, 0.05 mL, 0.66 mmol). The reaction mixture was stirred at rt for 21 h and diluted with DCM (80 mL). The organic layer was washed with saturated aqueous sodium bicarbonate (20 mL), dried over anhydrous sodium sulfate, filtered and evaporated under reduced pressure to give 3-bromo-1-tert-butyl-4-(methoxymethoxy)-2,5-dimethyl-benzene (70 mg, 80%) as a brown oil. ¹H NMR (400 MHz, CDCl₃) δ (ppm) 7.13 (s, 1H), 5.04 (s, 2H), 3.66 (s, 3H), 2.56 (s, 3H), 2.33 (s, 3H), 1.40 (s, 9H).

### Step 3: 1-tert-butyl-3-fluoro-4-(methoxymethoxy)-2,5-dimethyl-benzene

To a solution of 3-bromo-1-tert-butyl-4-(methoxymethoxy)-2,5-dimethyl-benzene (1.09 g, 3.61 mmol) in THF (20 mL) at −78° C. was slowly added a solution of n-BuLi in hexanes (1.7 mL of 2.5 M, 4.25 mmol) under nitrogen atmosphere. The mixture was then stirred at this temperature for 30 minutes after which, a solution of N-fluorobenzenesulfonimide (1.3 g, 4.12 mmol) in THF (20 mL) was added dropwise over 20 minutes. The resulting reaction mixture was stirred for 1 hour at −78° C. The reaction mixture was warmed to room temperature, diluted with water (20 mL) and extracted with ethyl acetate (2×40 mL). The combined organic layers were washed with water (40 mL), dried over anhydrous sodium sulfate, filtered and concentrated under reduced pressure. The crude product was purified by silica gel column chromatography using 0 to 20% ethyl acetate in heptanes to afford 1-tert-butyl-3-fluoro-4-(methoxymethoxy)-2,5-dimethyl-benzene (827 mg, 72%) as a clear oil. ¹H NMR (400 MHz, CDCl₃) δ (ppm) 6.93 (s, 1H), 5.10 (s, 2H), 3.62 (s, 3H), 2.39 (d, J=3.6 Hz, 3H), 2.29 (s, 3H), 1.40 (s, 9H). ¹⁹F NMR (376 MHz, CDCl₃) δ (ppm) −131.65 (s, 1F).

### Step 4: 4-tert-butyl-2-fluoro-3,6-dimethyl-phenol

Aqueous hydrochloric acid (3 mL of 6 M, 18 mmol) was added to a solution of 1-tert-butyl-3-fluoro-4-(methoxymethoxy)-2,5-dimethyl-benzene (827 mg, 2.62 mmol) in tetrahydrofuran (10 mL) and reaction mixture was stirred at room temperature for 18 hours. The reaction mixture was diluted with water (50 mL) and extracted using MTBE (3×50 mL). The organic layers were combined, washed with brine (50 mL), dried over sodium sulfate and concentrated under reduced pressure. The crude product was purified by silica gel chromatography using 0 to 20% ethyl acetate in heptanes to afford 4-tert-butyl-2-fluoro-3,6-dimethyl-phenol (508 mg, 99%) as a clear oil. ¹H NMR (400 MHz, CDCl₃) δ (ppm) 6.89 (s, 1H), 4.95 (d, J=5.7 Hz, 1H), 2.40 (d, J=3.4 Hz, 3H), 2.25 (s, 3H), 1.39 (s, 9H). ¹⁹F NMR (376 MHz, CDCl₃) δ (ppm) −142.65 (s, 1F).

### Step 5: 2-(4-tert-butyl-2-fluoro-3,6-dimethyl-phenyl)-4,4,5,5-tetramethyl-1,3,2-dioxaborolane (Intermediate B-43)

2-(4-tert-butyl-2-fluoro-3,6-dimethyl-phenyl)-4,4,5,5-tetramethyl-1,3,2-dioxaborolane (Intermediate B-43) was prepared from 4-tert-butyl-2-fluoro-3,6-dimethyl-phenol using procedure analogous to that found in Intermediate B-24 (Step 1). ESI-MS m/z calc. 306.22, found 307.3 (M+1)⁺. ¹H NMR (400 MHz, CDCl₃) δ (ppm) 6.96 (s, 1H), 2.42 (s, 3H), 2.36 (d, J=3.4 Hz, 3H), 1.43-1.38 (m, 21H).

### Intermediate B-44

### tert-butyl(5-tert-butyl-2-(4,4,5,5-tetramethyl-1,3,2-dioxaborolan-2-yl)phenoxy)dimethylsilane

### Step 1: 2-bromo-5-tert-butylphenol

To a stirred solution of 3-tert-butylphenol (300 g, 1997 mmol) in DCM (3 L) was added molecular bromine (325.53 g, 2037 mmol) dropwise at 0° C. under nitrogen atmosphere. The resulting mixture was stirred for 3 h at 0° C. under nitrogen atmosphere. The reaction was quenched with saturated sodium thiosulfate at 0° C. The aqueous layer was extracted with ethyl acetate. The combined organic layers were concentrated under reduced pressure. The residue was purified by silica gel column chromatography using 0 to 10% ethyl acetate in ether to afford 2-bromo-5-tert-butylphenol (295 g, 65%) LCMS: (ESI, m/z) [M−1]⁻=227.0.

### Step 2: 5-tert-butyl-2-(4,4,5,5-tetramethyl-1,3,2-dioxaborolan-2-yl)phenol

To a stirred mixture of 2-bromo-5-tert-butylphenol (295 g, 1288 mmol) and potassium acetate (189.54 g, 1931 mmol) in dioxane (2.8 L) and water (200 mL) was added potassium acetate (189.54 g, 1931 mmol), bis(pinacolato)diboron (360 g, 1416.3 mmol) and Pd(dppf)Cl₂ (47.11 g, 64.4 mmol) at room temperature under nitrogen atmosphere. The resulting mixture was stirred overnight at 90° C. under nitrogen atmosphere. The resulting mixture was concentrated under reduced pressure. The residue was purified by silica gel column chromatography using 0 to 10% ethyl acetate in ether to afford 5-tert-butyl-2-(4,4,5,5-tetramethyl-1,3,2-dioxaborolan-2-yl)phenol (135.5 g, 38%) as an off-white solid. ¹H-NMR (400 MHz, CDCl₃) δ (ppm) 7.78-7.75 (m, 1H), 7.53-7.51 (m, 1H), 6.97-6.88 (m, 1H), 1.35 (s, 12H), 1.30 (s, 9H).

### Step 3: tert-butyl(5-tert-butyl-2-(4,4,5,5-tetramethyl-1,3,2-dioxaborolan-2-yl)phenoxy)dimethylsilane

To a stirred mixture of 5-tert-butyl-2-(4,4,5,5-tetramethyl-1,3,2-dioxaborolan-2-yl)phenol (135 g, 489 mmol) and imidazole (37 g, 538 mmol) in dimethylformamide (2 L) was added TBSCl (81 g, 537 mmol) in portions at 0° C. under nitrogen atmosphere. The reaction was stirred at room temperature overnight. The reaction mixture was concentrated under reduced pressure and the residue was purified by silica gel column chromatography using 0 to 10% ethyl acetate in ether to afford tert-butyl(5-tert-butyl-2-(4,4,5,5-tetramethyl-1,3,2-dioxaborolan-2-yl)phenoxy)dimethylsilane (Intermediate B-44, 50 g, 26%) as a white solid. MS (ESI, m/z): [M+1]=391.35. ¹H NMR (400 MHz, CDCl₃) δ (ppm) 7.71-7.65 (m, 1H), 7.03-6.98 (m, 1H), 6.83-6.81 (m, 1H), 1.35 (s, 12H), 1.31 (s, 9H), 1.06 (s, 9H), 0.26 (s, 6H).

### Intermediate B-45

### 2-(4-tert-butyl-2,6-difluoro-phenyl)-4,4,5,5-tetramethyl-1,3,2-dioxaborolane

### Step 1: 2-(4-tert-butyl-2,6-difluoro-phenyl)-4,4,5,5-tetramethyl-1,3,2-dioxaborolane (Intermediate B-45)

n-BuLi in hexanes (0.3 mL of 1.6 M, 0.48 mmol) was added slowly to a stirring solution of 1-tert-butyl-3,5-difluoro-benzene (91 mg, 0.4 mmol) in THF (2 mL) at −78° C. under an atmosphere of argon. The mixture was stirred for 1 h before a solution of 2-isopropoxy-4,4,5,5-tetramethyl-1,3,2-dioxaborolane (118 mg, 0.13 mL, 0.64 mmol) in THF (1 mL) was added slowly. The reaction was warmed to room temperature over 1 h. The reaction mixture was quenched with water (10 mL) and extracted with ethyl acetate (10 mL). The organic extracts were washed with brine (10 mL), dried over magnesium sulfate and concentrated. Purification of the crude by reverse phase chromatography using 50 to 95% acetonitrile in water containing 0.1% formic acid gave 2-(4-tert-butyl-2,6-difluoro-phenyl)-4,4,5,5-tetramethyl-1,3,2-dioxaborolane (Intermediate B-45, 14 mg, 9%) as a white solid. ¹H-NMR (400 MHz, CDCl₃) δ (ppm) 6.88-6.83 (m, 2H), 1.38 (s, 12H), 1.28 (s, 9H). ¹⁹F-NMR (376 MHz, CDCl₃) δ (ppm) −101.0 (d, J=9.7 Hz, 2F).

### Intermediate B-46

### 2-[2-chloro-5-methyl-4-(4,4,5,5-tetramethyl-1,3,2-dioxaborolan-2-yl)phenyl]-2-methyl-propanoic acid

### Step 1: 2-(4-bromo-2-chloro-5-methyl-phenyl)propan-2-ol

To a solution of 1,4-dibromo-2-chloro-5-methyl-benzene (504 mg, 1.68 mmol) in diethyl ether (13 mL), cooled to −78° C. was added n-Buli (0.7 mL of 2.5 M in hexanes, 1.75 mmol). The mixture was stirred at −78° C. for 1 h. Acetone (0.4 mL, 5.5 mmol) was added dropwise and stirring continued for 90 min at −78° C. The dry-ice bath was removed and the reaction was immediately quenched by slow addition of saturated aqueous ammonium chloride (20 mL). The layers were separated and the aqueous layer was extracted with MTBE (2×15 mL). The combined organic extracts were dried over sodium sulfate, filtered and concentrated under reduced pressure. Purification by silica gel chromatography (0-10% ethyl acetate/heptanes) provided 2-(4-bromo-2-chloro-5-methyl-phenyl)propan-2-ol (367 mg, 77%). ESI-MS m/z calc. 261.98, found 245.1 (M−17)⁺.

### Step 2: 2-(4-bromo-2-chloro-5-methyl-phenyl)-2-methyl-propanenitrile

To a solution of trimethylsilylformonitrile (79 mg, 0.1 mL, 0.78 mmol) and indium bromide (15 mg, 0.04 mmol) in DCM (0.7 mL) at room temperature was added dropwise a solution of 2-(4-bromo-2-chloro-5-methyl-phenyl)propan-2-ol (100 mg, 0.36 mmol) in DCM (0.7 mL) over 5 min and the solution stirred at room temperature for 2.5 h. Additional trimethylsilylformonitrile (79.3 mg, 0.1 mL, 0.78 mmol) and indium bromide (13 mg, 0.038 mmol) were then added and the mixture was stirred at room temperature for 1 h. The mixture was concentrated under reduced pressure and then co-evaporated with DCM (2×20 mL) to provide 2-(4-bromo-2-chloro-5-methyl-phenyl)-2-methyl-propanenitrile (103 mg, 71%) as a brown oil. ESI-MS m/z calc. 270.97, found 272.0 (M+1)⁺. ¹H NMR (400 MHz, CDCl₃) δ (ppm) 7.63 (s, 1H), 7.34 (s, 1H), 2.40 (s, 3H), 1.86 (s, 6H).

### Step 3: 2-(4-bromo-2-chloro-5-methyl-phenyl)-2-methyl-propanoic acid

Potassium hydroxide (4 mL of 40% w/v, 29 mmol) was added to a solution of 2-(4-bromo-2-chloro-5-methyl-phenyl)-2-methyl-propanenitrile (450 mg, 1.49 mmol) in diethylene glycol (3 mL) and the mixture was stirred at 150° C. for 3 days in a sealed tube. The mixture was cooled to room temperature, diluted with water (30 mL) and washed with MTBE (20 mL). The aqueous layer was acidified to pH 1-2 using 3 M aqueous HCl and extracted with MTBE (3×30 mL). The combined extracts were dried over sodium sulfate, filtered and concentrated under reduced pressure. Purification by reverse phase chromatography (5-95% acetonitrile/water (0.1% formic acid) provided 2-(4-bromo-2-chloro-5-methyl-phenyl)-2-methyl-propanoic acid (140 mg, 32%) as an off-white solid. ESI-MS m/z calc. 289.97, found 291.0 (M+1)⁺. ¹H NMR (400 MHz, CDCl₃) δ (ppm) 7.58 (s, 1H), 7.29 (s, 1H, overlapping with solvent peak), 2.42 (s, 3H), 1.66 (s, 6H). ESI-MS m/z calc. 289.97, found 291.0 (M+1)⁺.

### Step 4: 2-[2-chloro-5-methyl-4-(4,4,5,5-tetramethyl-1,3,2-dioxaborolan-2-yl)phenyl]-2-methyl-propanoic acid (Intermediate B-46)

2-[2-Chloro-5-methyl-4-(4,4,5,5-tetramethyl-1,3,2-dioxaborolan-2-yl)phenyl]-2-methyl-propanoic acid (Intermediate B-46, 0.76 g, 55%) was prepared using procedure analogous to Intermediate B-1 (Step 2) as a brown solid. ESI-MS m/z calc. 338.15, found 339.2 (M+1)⁺. ¹H NMR (400 MHz, CDCl₃) δ (ppm) 7.77 (s, 1H), 7.22 (s, 1H), 2.55 (s, 3H), 1.66 (s, 6H), 1.35 (s, 12H).

### Intermediate B-47

### 2-[2-chloro-5-methyl-4-(4,4,5,5-tetramethyl-1,3,2-dioxaborolan-2-yl)phenyl]-2-methyl-propan-1-ol

### Step 1: 2-(4-bromo-2-chloro-5-methyl-phenyl)-2-methyl-propan-1-ol

To a solution of 2-(4-bromo-2-chloro-5-methyl-phenyl)-2-methyl-propanoic acid (1.49 g, 5.11 mmol) in THF (30 mL) at 0° C. was added borane-THF complex solution in THF (11 mL of 1 M, 11 mmol) and the solution was stirred at 70° C. for 1 h. Once cooled to room temperature, the reaction mixture was quenched with addition of 1N HCl aqueous solution (30 mL) and extracted with ethyl acetate (3×50 mL). The combined organic layer was washed with brine (50 mL), dried over anhydrous sodium sulfate, filtered and concentrated under reduced pressure to afford 2-(4-bromo-2-chloro-5-methyl-phenyl)-2-methyl-propan-1-ol (1.4 g, 99%) as a clear oil. ESI-MS m/z calc. 275.99, found 259.0 (M−17)⁺. H NMR (400 MHz, CDCl₃) δ (ppm) 7.55 (s, 1H), 7.31 (s, 1H), 3.99 (s, 2H), 2.39 (s, 3H), 1.60 (brs, 1H), 1.47 (s, 6H).

### Step 2: 2-[2-chloro-5-methyl-4-(4,4,5,5-tetramethyl-1,3,2-dioxaborolan-2-yl)phenyl]-2-methyl-propan-1-1 (Intermediate B-47)

2-[2-chloro-5-methyl-4-(4,4,5,5-tetramethyl-1,3,2-dioxaborolan-2-yl)phenyl]-2-methyl-propan-1-ol (1.04 g, 59%) was prepared using procedure analogous to Intermediate B-1 (Step 2) as a white solid. ESI-MS m/z calc. 324.17, found 325.2 (M+1)⁺. ¹H NMR (400 MHz, CDCl₃) δ (ppm) 7.75 (s, 1H), 7.25 (s, 1H), 4.01 (d, J=6.4 Hz, 2H), 2.52 (s, 3H), 1.49 (s, 6H), 1.35 (s, 12H).

### Intermediate B-48

### 2-[3-chloro-6-methyl-5-(4,4,5,5-tetramethyl-1,3,2-dioxaborolan-2-yl)-2-pyridyl]-2-methyl-propan-1-ol

### Step 1: 2,5-dibromo-3-chloro-6-methyl-pyridine

To a suspension of 5-bromo-3-chloro-6-methyl-pyridin-2-ol (1.49 g, 6.1 mmol) in toluene (25 mL) was added phosphorus oxybromide (2.52 g, 8.8 mmol). The reaction mixture was placed in a pre-heated oil bath set at 100° C. and stirred for 21 hours. Once cooled to room temperature, the reaction mixture was poured into a saturated aqueous sodium bicarbonate solution (200 mL) and ethyl acetate (100 mL) and stirred vigorously for 15-30 minutes. The layers were separated and the aqueous layer was extracted with ethyl acetate (2×100 mL). The combined organic layer was washed with saturated aqueous sodium bicarbonate (100 mL), brine (100 mL), dried over sodium sulfate, filtered and concentrated under reduced pressure. Purification by silica gel column chromatography using 0 to 10% EtOAc in heptane afforded 2,5-dibromo-3-chloro-6-methyl-pyridine (1.59 g, 83%) as an orange solid. ESI-MS m/z calc. 282.84, found 283.8 (M+1)⁺. H NMR (400 MHz, CDCl₃) δ 7.87 (s, 1H), 2.64 (s, 3H).

### Step 2: methyl 2-(5-bromo-3-chloro-6-methyl-2-pyridyl)-2-methyl-propanoate

To a flame-dried flask was added diisopropylamine (910 mg, 1.26 mL, 9 mmol) and THF (20 mL). The solution was cooled to −78° C. and treated with a n-BuLi solution in hexanes (3.6 mL of 2.5 M, 9.mmol). The flask was transferred to an ice/water bath and stirred for 20 minutes before being cooled again to −78° C. Methyl 2-methylpropanoate (918 mg, 1.03 mL, 9 mmol) was added and the reaction mixture was stirred at −78° C. After 1 hour, a solution of 2,5-dibromo-3-chloro-6-methyl-pyridine (939 mg, 3 mmol) in THF (4 mL) was added, the cold bath was removed and the reaction mixture was stirred at room temperature for 18 hours. The crude mixture was partitioned between saturated aqueous ammonium chloride (100 mL) and ethyl acetate (75 mL), the layers were separated and the aqueous layer was extracted with ethyl acetate (75 mL). The combined organic layer was washed with brine (75 mL), dried over sodium sulfate, filtered and concentrated under reduced pressure. Purification by silica gel column chromatography using 0 to 10% EtOAc in heptane afforded methyl 2-(5-bromo-3-chloro-6-methyl-2-pyridyl)-2-methyl-propanoate (515 mg, 53%) as a pale yellow solid. ESI-MS m/z calc. 304.98, found 306.0 (M+1)⁺. ¹H NMR (400 MHz, CDCl₃) δ 7.76 (s, 1H), 3.69 (s, 3H), 2.63 (s, 3H), 1.61 (s, 6H).

### Step 3: 2-(5-bromo-3-chloro-6-methyl-2-pyridyl)-2-methyl-propan-1-ol

To a solution of methyl 2-(5-bromo-3-chloro-6-methyl-2-pyridyl)-2-methyl-propanoate (132 mg, 0.37 mmol) in THF (4 mL) cooled to 0° C. in an ice/water bath was added dropwise diisobutylaluminum hydride solution in toluene (1.9 mL of 1 M, 1.9 mmol). The ice bath was removed and the reaction was stirred at room temperature for 5 hours. The crude reaction mixture was quenched by the addition of 1 M aqueous Rochelle salt solution (6-8 mL) and further diluted with MTBE (6 mL). The biphasic mixture was stirred vigorously at room temperature until both layers were clear. The mixture was then partitioned between 1 M aqueous Rochelle salt solution (30 mL) and MTBE (30 mL) and the layers were separated. The aqueous layer was extracted with MTBE (30 mL). The combined organic layer was washed with brine (40 mL), dried over sodium sulfate, filtered and concentrated under reduced pressure. Purification by reversed-phase chromatography (C₁₈) using 5-100% MeCN in water (0.1% formic acid) afforded 2-(5-bromo-3-chloro-6-methyl-2-pyridyl)-2-methyl-propan-1-ol (61 mg, 59%) as a white solid. ESI-MS m/z calc. 276.99, found 278.0 (M+1)⁺. ¹H NMR (400 MHz, CDCl₃) δ 7.82 (s, 1H), 4.18 (t, J=7.0 Hz, 1H), 3.77 (d, J=6.8 Hz, 2H), 2.62 (s, 3H), 1.45 (s, 6H).

### Step 4: 2-[3-chloro-6-methyl-5-(4,4,5,5-tetramethyl-1,3,2-dioxaborolan-2-yl)-2-pyridyl]-2-methyl-propan-1-ol Intermediate B-48)

2-[3-chloro-6-methyl-5-(4,4,5,5-tetramethyl-1,3,2-dioxaborolan-2-yl)-2-pyridyl]-2-methyl-propan-1-ol (Intermediate B-48, 40.7 mg, 47%) was prepared from 2-(5-bromo-3-chloro-6-methyl-2-pyridyl)-2-methyl-propan-1-ol using procedure analogous to that found in Intermediate B-1, Step 2. ESI-MS m/z calc. 325.16, found 326.2 (M+1)⁺. ¹H NMR (400 MHz, CDCl₃) δ 7.97 (s, 1H), 4.88 (br. s, 1H), 3.77 (br. s, 2H), 2.68 (s, 3H), 1.45 (s, 6H), 1.35 (s, 12H).

### Intermediate B-49

### 5-chloro-2-methyl-3-(4,4,5,5-tetramethyl-1,3,2-dioxaborolan-2-yl)-6-(2,2,2-trifluoro-1,1-dimethyl-ethyl) pyridine

### Step 1: 5-chloro-2-methyl-6-(2,2,2-trifluoro-1,1-dimethyl-ethyl)pyridine-3-carboxylic acid

5-chloro-2-methyl-6-(2,2,2-trifluoro-1,1-dimethyl-ethyl)pyridine-3-carboxylic acid was prepared from 5-chloro-2-methyl-6-(2,2,2-trifluoro-1,1-dimethyl-ethyl)pyridine-3-carbonitrile using a procedure analogous to that found in Intermediate B-7 (Step 1 and Step 2), using 4,4,4-trifluoro-3,3-dimethylbutan-2-one, followed by procedure analogous to that found in Intermediate B-12 (Step 1 to Step 3). ESI-MS m/z calc. 281.04, found 282.1 (M+1)⁺. ¹H NMR (400 MHz, DMSO-d₆) δ 13.63 (br. s, 1H), 8.19 (s, 1H), 2.69 (s, 3H), 1.74 (s, 6H). ¹⁹F NMR (377 MHz, DMSO-d₆) δ −72.42 (s, 3F).

### Step 2: 5-chloro-2-methyl-3-(4,4,5,5-tetramethyl-1,3,2-dioxaborolan-2-yl)-6-(2,2,2-trifluoro-1,1-dimethyl-ethyl)pyridine (Intermediate B-49)

To a flame dried sealed tube, was added 5-chloro-2-methyl-6-(2,2,2-trifluoro-1,1-dimethyl-ethyl)pyridine-3-carboxylic acid (3.61 g, 12.44 mmol), 4,4,5,5-tetramethyl-2-(4,4,5,5-tetramethyl-1,3,2-dioxaborolan-2-yl)-1,3,2-dioxaborolane (4.73 g, 18.63 mmol), triethylamine (2.27 g, 3.15 mL, 22.41 mmol) and trimethylacetic anhydride (3.78 g, 4.15 mL, 20.28 mmol) in 1,4-dioxane (36 mL). The tube was purged with nitrogen gas for 10 minutes and 1,4-bis(diphenylphosphino)butane (625 mg, 1.46 mmol) and palladium acetate (210 mg, 0.94 mmol) were added. The tube was sealed and heated at 160° C. for 6 h. After cooling the reaction mixture to room temperature, it was filtered over Celite®, rinsed with dichloromethane (500 mL) and concentrated under reduced pressure. The residue was purified by silica gel column chromatography using 0 to 5% EtOAc in heptanes to obtain 5-chloro-2-methyl-3-(4,4,5,5-tetramethyl-1,3,2-dioxaborolan-2-yl)-6-(2,2,2-trifluoro-1,1-dimethyl-ethyl) pyridine (Intermediate B-49, 790 mg, 17%) as yellow solid ESI-MS m/z calc. 363.14, found 364.2 (M+1)⁺. ¹H NMR (400 MHz, CDCl₃) δ 7.97 (s, 1H), 2.68 (s, 3H), 1.76 (s, 6H), 1.35 (s, 12H). ¹⁹F NMR (377 MHz, CDCl₃) δ −73.53 (s, 3F).

### Intermediate B-50

### [6-(1-bicyclo[1.1.1]pentanyl)-5-chloro-2-methyl-3-pyridyl]boronic acid

### Step 1: 2-(1-bicyclo[1.1.1]pentanyl)-5-bromo-3-chloro-6-methyl-pyridine

3-Bromo-5-chloro-2-methyl-pyridine (227 mg, 1.1 mmol), bicyclo[1.1.1]pentane-1-carboxylic acid (100 mg, 0.9 mmol), AgNO3 (231 mg, 1.34 mmol), and ammonium persulfate (410 mg, 1.8 mmol) were combined in a flask, to which was added a mixture of acetonitrile (2.5 mL) and water (2.5 mL). The reaction mixture was heated at 60° C. for 1 hour, filtered, diluted with ethyl acetate, washed with a saturated aqueous solution of ammonium chloride and brine. The organic layer was dried over anhydrous magnesium sulfate, filtered, and concentrated under reduced pressure. Purification by silica gel column chromatography using 0 to 20% of EtOAc in hexanes gave 2-(1-bicyclo[1.1.1]pentanyl)-5-bromo-3-chloro-6-methyl-pyridine (137 mg, 56%) ESI-MS m/z calc. 270.97, found 272.05 (M+1)⁺.

### Step 2: [6-(1-bicyclo[1.1.1]pentanyl)-5-chloro-2-methyl-3-pyridyl]boronic acid (Intermediate B-50)

A microwave vial was charged with 2-(1-bicyclo[1.1.1]pentanyl)-5-bromo-3-chloro-6-methyl-pyridine (137 mg, 0.50 mmol) and Et₂O (2 mL) was cooled to −78° C. After 20 minutes n-BuLi (390 μL of 2.5 M, 0.98 mmol) was added dropwise and the resulting mixture was stirred for 25 minutes at −78° C., followed by the dropwise addition of trimethyl borate (400 μL, 3.52 mmol). The resulting mixture was allowed to stir for 15 minutes at −78° C. and then it was quenched with saturated aqueous solution of ammonium chloride, diluted with ethyl acetate, and washed with brine. The organic layer was dried over magnesium sulfate, filtered and concentrated under reduced pressure to give [6-(1-bicyclo[1.1.1]pentanyl)-5-chloro-2-methyl-3-pyridyl]boronic acid (Intermediate B-50) which was used as crude for the next step. ESI-MS m/z calc. 237.07, found 238.0 (M+1)⁺.

### Intermediate B-51

### (5-chloro-2-methyl-6-spiro[3.3]heptan-2-yl-3-pyridyl)boronic acid

### Step 1: (5-chloro-2-methyl-6-spiro[3.3]heptan-2-yl-3-pyridyl)boronic acid

### Step 1: (5-chloro-2-methyl-6-spiro[3.3]heptan-2-yl-3-pyridyl)boronic acid (Intermediate B-51)

(5-chloro-2-methyl-6-spiro[3.3]heptan-2-yl-3-pyridyl)boronic acid was prepared from spiro[3.3]heptane-2-carboxylic acid (Intermediate B-51) using procedure analogous to that found in Intermediate B-50 (Step 1 and Step 2). ESI-MS m/z calc. 265.10, found 266.0 (M+1)⁺.

### Intermediate B-52

### [6-(1-bicyclo[2.1.1]hexanyl)-5-chloro-2-methyl-3-pyridyl]boronic acid

### Step 1: [6-(1-bicyclo[2.1.1]hexanyl)-5-chloro-2-methyl-3-pyridyl]boronic acid (Intermediate B-52)

[6-(1-bicyclo[2.1.1]hexanyl)-5-chloro-2-methyl-3-pyridyl]boronic acid (Intermediate B-52) was prepared from bicyclo[2.1.1]hexane-1-carboxylic acid using procedure analogous to that found in Intermediate B-50 (Step 1 and Step 2). ESI-MS m/z calc. 251.09, found 252.0 (M+1)⁺.

### Intermediate B-53

### 3-chloro-2-(3,3-difluoro-1-methyl-cyclobutyl)-6-methyl-5-(4,4,5,5-tetramethyl-1,3,2-dioxaborolan-2-yl)pyridine

### Step 1: methyl 5-chloro-6-(3,3-difluoro-1-methyl-cyclobutyl)-2-methyl-pyridine-3-carboxylate

A mixture of methyl 5-chloro-2-methyl-pyridine-3-carboxylate (754 mg, 4.06 mmol), 3,3-difluoro-1-methyl-cyclobutanecarboxylic acid (625 mg, 4.08 mmol), AgNO3 (1.4 g, 8.14 mmol) and ammonium persulfate (1.89 g, 8.12 mmol) in acetonitrile (12 mL) and water (12 mL) was heated at 60° C. for 90 min. The reaction mixture was cooled then partitioned between ethyl acetate and water. The organic layer was separated, washed with brine, dried over sodium sulfate, filtered and concentrated. Purification by silica gel chromatography using 0 to 20% ethyl acetate in hexane provided methyl 5-chloro-6-(3,3-difluoro-1-methyl-cyclobutyl)-2-methyl-pyridine-3-carboxylate (690 mg, 59%). ESI-MS m/z calc. 289.07, found 290.2 (M+1)⁺. ¹H NMR (400 MHz, CD₃OD) δ 8.21 (s, 1H), 3.91 (s, 3H), 3.28-3.14 (m, 2H), 2.75 (s, 3H), 2.77-2.68 (m, 2H), 1.59 (s, 3H).

### Step 2: 5-chloro-6-(3,3-difluoro-1-methyl-cyclobutyl)-2-methyl-pyridine-3-carboxylic acid

A solution of methyl 5-chloro-6-(3,3-difluoro-1-methyl-cyclobutyl)-2-methyl-pyridine-3-carboxylate (680 mg, 2.35 mmol) in methanol (20 mL) at 45° C. was treated dropwise with aqueous NaOH (12 mL of 1 M, 12 mmol) and stirred for 30 minutes. The mixture was partitioned between ethyl acetate and aqueous 1 M HCl (30 mL) and the layers were separated. The aqueous layer extracted with ethyl acetate (2×). The combined organic layer was washed with brine, dried over sodium sulfate, filtered and concentrated to provide 5-chloro-6-(3,3-difluoro-1-methyl-cyclobutyl)-2-methyl-pyridine-3-carboxylic acid (640 mg, 99%). ESI-MS m/z calc. 275.05, found 276.1 (M+1)⁺. ¹H NMR (400 MHz, DMSO-d₆) δ 13.48 (s, 1H), 8.16 (s, 1H), 3.29-3.13 (m, 2H), 2.84-2.72 (m, 2H), 2.70 (s, 3H), 1.55 (s, 3H).

### Step 3: 3-chloro-2-(3,3-difluoro-1-methyl-cyclobutyl)-6-methyl-5-(4,4,5,5-tetramethyl-1,3,2-dioxaborolan-2-yl)pyridine (Intermediate B-53)

To a microwave vial equipped with a stir bar was added 5-chloro-6-(3,3-difluoro-1-methyl-cyclobutyl)-2-methyl-pyridine-3-carboxylic acid (57 mg, 0.21 mmol), bis(pinacol)diboron (79 mg, 0.31 mmol), diacetoxypalladium (2.3 mg, 0.01 mmol), 4-diphenylphosphanylbutyl(diphenyl)phosphane (9 mg, 0.02 mmol), trimethylacetic anhydride (63 μL, 0.31 mmol) and TEA (43 μL, 0.31 mmol). The vial was sealed, placed under nitrogen positive pressure and subjected to three evacuation/backfill cycles under high vacuum. Nitrogen-degassed dioxane (1.2 mL) was added and the vial placed in a pre-heated heating block at 170° C. The mixture was stirred vigorously for 16 h, then cooled to room temperature and partitioned between ethyl acetate and saturated ammonium chloride. The layers were separated and the organic layer washed with water and brine, dried over sodium sulfate, filtered and concentrated to provide 3-chloro-2-(3,3-difluoro-1-methyl-cyclobutyl)-6-methyl-5-(4,4,5,5-tetramethyl-1,3,2-dioxaborolan-2-yl)pyridine (Intermediate B-53, 73 mg, 99%). ESI-MS m/z calc. 357.15, found 358.2 (M+1)⁺.

### Intermediate B-54

### 5-chloro-2-methyl-6-(1-methylcyclopentyl)-3-(4,4,5,5-tetramethyl-1,3,2-dioxaborolan-2-yl)pyridine

### Step 1: 5-chloro-2-methyl-6-(1-methylcyclopentyl)-3-(4,4,5,5-tetramethyl-1,3,2-dioxaborolan-2-yl)pyridine (Intermediate B-54)

5-chloro-2-methyl-6-(1-methylcyclopentyl)-3-(4,4,5,5-tetramethyl-1,3,2-dioxaborolan-2-yl)pyridine (Intermediate B-54) was prepared from 1-methylcyclopentanecarboxylic acid using procedure analogous to that found in Intermediate B-53 (Step 1 to Step 3). ESI-MS m/z calc. 335.18, found 336.3 (M+1)⁺.

### Intermediate B-55

### (5-chloro-2-methyl-6-norboman-1-yl-3-pyridyl)boronic acid

### Step 1: (5-chloro-2-methyl-6-norboman-1-yl-3-pyridyl)boronic acid (Intermediate B-55)

(5-chloro-2-methyl-6-norboman-1-yl-3-pyridyl)boronic acid (Intermediate B-55) was prepared from Norbornane-1-carboxylic acid using procedure analogous to that found in Intermediate B-53 (Step 1 to Step 3). ESI-MS m/z calc. 265.10, found 266.2 (M+1). (6-(bicyclo[2.2.1]heptan-2-yl)-5-chloro-2-methylpyridin-3-yl)boronic acid was also isolated.

### Intermediate B-56

### 5-chloro-2-methyl-6-phenyl-3-(4,4,5,5-tetramethyl-1,3,2-dioxaborolan-2-yl)pyridine

### Step 1: 3-chloro-6-methyl-2-phenyl-pyridine

A reaction flask was charged with phenylboronic acid (593 mg, 4.86 mmol), 2-bromo-3-chloro-6-methyl-pyridine (1 g, 4.84 mmol), PdCl₂(dtbpf) (158 mg, 0.24 mmol), and potassium phosphate (4.73 g, 22.28 mmol), dioxane (50 mL) and water (12.5 mL). The resulting reaction mixture was degassed under an atmosphere of nitrogen for 5 minutes and heated at 45° C. for 2 hours. The resulting reaction mixture was diluted with ethyl acetate, washed with a saturated aqueous solution of ammonium chloride and brine. The organic layer was dried over anhydrous magnesium sulfate, filtered, and concentrated under reduced pressure. Purification by silica gel flash chromatography using 0 to 100% EtOAc in hexanes gave 3-chloro-6-methyl-2-phenyl-pyridine (845 mg, 79%) as an orange oil. ESI-MS m/z calc. 203.05, found 204.09 (M+1)⁺. ¹H NMR (400 MHz, CDCl₃) δ 7.72-7.67 (m, 2H), 7.66 (d, J=8.2 Hz, 1H), 7.49-7.38 (m, 3H), 7.08 (d, J=8.2 Hz, 1H), 2.59 (s, 3H).

### Step 2: 3-bromo-5-chloro-2-methyl-6-phenyl-pyridine

Bromine (approximately 9.13 g, 2.94 mL, 57.11 mmol) was added to 3-chloro-6-methyl-2-phenyl-pyridine (840 mg, 3.81 mmol) dissolved in acetic acid (5 mL). The reaction mixture was stirred at 70° C. for 2 hours. It was poured onto a saturated solution of sodium thiosulfate pentahydrate (400 mL) and sodium carbonate (400 mL) then extracted with ethyl acetate (2×800 mL). The combined organic extracts were washed with brine (500 mL) and dried over magnesium sulfate, filtered and concentrated under reduced pressure. Purification by high pressure reverse phase chromatography (C₁₈) using 1 to 100% ACN in water (5 mM hydrochloric acid) gave 3-bromo-5-chloro-2-methyl-6-phenyl-pyridine (240 mg, 22%) as a yellow solid. ESI-MS m/z calc. 280.96, found 282.012 (M+1)⁺.

### Step 3: 5-chloro-2-methyl-6-phenyl-3-(4,4,5,5-tetramethyl-1,3,2-dioxaborolan-2-yl)pyridine (Intermediate B-56)

5-chloro-2-methyl-6-phenyl-3-(4,4,5,5-tetramethyl-1,3,2-dioxaborolan-2-yl)pyridine (Intermediate B-56) was prepared from 3-bromo-5-chloro-2-methyl-6-phenyl-pyridine using procedure analogous to that found in Intermediate B-1, Step 2. ESI-MS m/z calc. 329.13, found 248.11 (M−81)⁺.

### Intermediate B-57

### 3-chloro-2-(3,3-difluoro-1-methyl-cyclopentyl)-6-methyl-5-(4,4,5,5-tetramethyl-1,3,2-dioxaborolan-2-yl)pyridine

### Step 1: 3-chloro-2-(3,3-difluoro-1-methyl-cyclopentyl)-6-methyl-5-(4,4,5,5-tetramethyl-1,3,2-dioxaborolan-2-yl)pyridine (Intermediate B-57)

3-chloro-2-(3,3-difluoro-1-methyl-cyclopentyl)-6-methyl-5-(4,4,5,5-tetramethyl-1,3,2-dioxaborolan-2-yl)pyridine (Intermediate B-57) was prepared from racemic 3,3-difluoro-1-methylcyclopentanecarboxylic acid using procedure analogous to that found in Intermediate B-53 (Step 1 to Step 3). ESI-MS m/z calc. 371.16, found 372.3 (M+1)⁺.

### Intermediate B-58

### (5-chloro-6-dispiro[2.0.2⁴.1³]heptan-7-yl-2-methyl-3-pyridyl)boronic acid

### Step 1: (5-chloro-6-dispiro[2.0.2⁴.1³]heptan-7-yl-2-methyl-3-pyridyl)boronic acid (Intermediate B-58)

(5-chloro-6-dispiro[2.0.2⁴.1³]heptan-7-yl-2-methyl-3-pyridyl)boronic acid (Intermediate B-58) was prepared from dispiro[2.0.24.13]heptane-7-carboxylic acid using procedure analogous to that found in Intermediate B-53 (Step 1 to Step 3). ESI-MS m/z calc. 263.09, found 264.18 (M+1)⁺.

### Intermediate B-59

### 5-chloro-2-methyl-6-(1-methylcyclopropyl)-3-(4,4,5,5-tetramethyl-1,3,2-dioxaborolan-2-yl)pyridine

### Step 1: 5-chloro-6-isopropenyl-2-methyl-pyridin-3-amine

To 6-bromo-5-chloro-2-methyl-pyridin-3-amine (2 g, 8.85 mmol) in 1,4-dioxane (18.75 mL) and water (6.25 mL) was added 2-isopropenyl-4,4,5,5-tetramethyl-1,3,2-dioxaborolane (1.6 g, 9.52 mmol), potassium carbonate (2.5 g, 18.09 mmol). The reaction mixture was degassed under an atmosphere of nitrogen for 3 minutes and Pd(dppf)Cl₂ (340 mg, 0.46 mmol) was added. The reaction mixture was heated at 90° C. for 16 h. Additional 2-isopropenyl-4,4,5,5-tetramethyl-1,3,2-dioxaborolane (300 mg, 1.78 mmol) was added and the reaction was degassed with argon for 5 minutes before Pd(dppf)Cl₂ (100 mg, 0.14 mmol) was added. The reaction was stirred at 100° C. for 3.5 h. The reaction mixture was cooled to room temperature, diluted with EtOAc (80 mL), washed with saturated aqueous NaHCO₃ (80 mL), water (80 mL), brine (80 mL), dried over Na₂SO₄ and concentrated under reduced pressure. Purification by silica gel column chromatography using 0 to 50% EtOAc in heptane yielded 5-chloro-6-isopropenyl-2-methyl-pyridin-3-amine (1.61 g, 95%) as an off white solid. ESI-MS m/z calc. 182.06, found 183.05 (M+1)⁺. ¹H-NMR (400 MHz, CDCl₃) δ 6.99 (s, 1H), 5.39 (t, J=1.5 Hz, 1H), 5.25 (s, 1H), 3.69 (s, 2H), 2.42 (s, 3H), 2.16 (s, 3H).

### Step 2: 3-chloro-5-iodo-2-isopropenyl-6-methyl-pyridine

To a cooled solution of 5-chloro-6-isopropenyl-2-methyl-pyridin-3-amine (2.83 g, 15.12 mmol) in 2N aqueous hydrochloric acid (50 mL) was added dropwise a solution of sodium nitrite (1.54 g, 22.32 mmol) in water (13 mL) at −5° C. After stirring below 0° C. for 5 minutes, the solution was added to a mixture of sodium iodide (4.53 g, 30.22 mmol), water (50 mL) and dichloromethane (50 mL) at 0-2° C. The reaction was stirred at 0-2° C. for 10 minutes and then warmed to room temperature over 1 hour. The reaction mixture was diluted with dichloromethane (150 mL) and water (100 mL). The layers were separated and the aqueous layer was further extracted with dichloromethane (100 mL). The combined organic extracts were washed with 10% sodium thiosulfate solution (100 mL), brine (100 mL), dried using magnesium sulfate, filtered and concentrated. Purification by silica gel column chromatography using 0 to 5% ethyl acetate in heptane gave 3-chloro-5-iodo-2-isopropenyl-6-methyl-pyridine (2.86 g, 61%) as a pale-yellow oil. ESI-MS m/z calc. 292.95, found 293.93 (M+1)⁺. ¹H-NMR (400 MHz, CDCl₃) δ 8.04 (s, 1H), 5.44 (s, 1H), 5.30 (s, 1H), 2.69 (s, 3H), 2.13 (s, 3H).

### Step 3: 5-chloro-3-iodo-2-methyl-6-(1-methylcyclopropyl)pyridine

To a solution of diethyl zinc in hexane (37 mL of 1 M, 37 mmol) in dichloromethane (90 mL) at 0° C. was added diiodomethane (9.98 g, 3 mL, 37.24 mmol) dropwise. After 30 minutes a solution of 3-chloro-5-iodo-2-isopropenyl-6-methyl-pyridine (2.86 g, 9.24 mmol) in dichloromethane (18 mL) was added dropwise and the reaction mixture was stirred at room temperature for 2 hours. The mixture was quenched with saturated ammonium chloride solution (100 mL), then partitioned between dichloromethane (500 mL) and water (250 mL). The organic layer was washed with brine (200 mL), dried over magnesium sulfate, filtered and concentrated. Purification by silica gel column chromatography using 0-3% ethyl acetate in heptane gave 5-chloro-3-iodo-2-methyl-6-(1-methylcyclopropyl)pyridine (2.35 g, 79%) as an orange oil. ESI-MS m/z calc. 306.96, found 307.94 (M+1)⁺. ¹H-NMR (400 MHz, CDCl₃) δ 7.97 (s, 1H), 2.66 (s, 3H), 1.39 (s, 3H), 0.97-0.93 (s, 2H), 0.82-0.77 (m, 2H).

### Step 4: 5-chloro-2-methyl-6-(1-methylcyclopropyl)-3-(4,4,5,5-tetramethyl-1,3,2-dioxaborolan-2-yl)pyridine (Intermediate B-59)

5-chloro-2-methyl-6-(1-methylcyclopropyl)-3-(4,4,5,5-tetramethyl-1,3,2-dioxaborolan-2-yl)pyridine (Intermediate B-59) was prepared from 5-chloro-3-iodo-2-methyl-6-(1-methylcyclopropyl)pyridine using procedure analogous to that found in Intermediate B-1, Step 2. ESI-MS m/z calc. 307.15, found 308.14 (M+1)⁺. ¹H-NMR (400 MHz, CDCl₃) δ 7.93 (s, 1H), 7.25 (s, 1H), 2.68 (s, 3H), 1.42 (s, 3H), 1.32 (s, 12H), 0.97 (dd, J=6.4, 4.6 Hz, 2H), 0.80 (dd, J=6.2, 4.4 Hz, 2H).

### Intermediate B-60

### 2-(1-bicyclo[2.2.2]octanyl)-3-chloro-6-methyl-5-(4,4,5,5-tetramethyl-1,3,2-dioxaborolan-2-yl)pyridine

### Step 1: 2-(1-bicyclo[2.2.2]octanyl)-3-chloro-6-methyl-5-(4,4,5,5-tetramethyl-1,3,2-dioxaborolan-2-yl)pyridine (Intermediate B-60)

2-(1-bicyclo[2.2.2]octanyl)-3-chloro-6-meth yl-5-(4,4,5,5-tetramethyl-1,3,2-dioxaborolan-2-yl)pyridine (Intermediate B-60) was prepared from bicyclo[2.2.2]octane-1-carboxylic acid using procedure analogous to that found in Intermediate B-53 (Step 1 to Step 3). ¹H NMR (400 MHz, CDCl₃-d) δ 7.87 (s, 1H), 2.65 (s, 3H), 2.14-2.05 (m, 6H), 1.71-1.64 (m, 7H), 1.33 (s, 12H). ESI-MS m/z calc. 361.198, found 362.2 (M+1)⁺.

### Intermediate B-61

### 5-chloro-2-methyl-6-(1-methylcyclobutyl)-3-(4,4,5,5-tetramethyl-1,3,2-dioxaborolan-2-yl)pyridine

### Step 1: 5-chloro-2-methyl-6-(1-methylcyclobutyl)-3-(4,4,5,5-tetramethyl-1,3,2-dioxaborolan-2-yl)pyridine (Intermediate B-61)

5-chloro-2-methyl-6-(1-methylcyclobutyl)-3-(4,4,5,5-tetramethyl-1,3,2-dioxaborolan-2-yl)pyridine (Intermediate B-61) was prepared from 1-methylcyclobutanecarboxylic acid, using procedure analogous to that found in Intermediate B-53 (Step 1 to Step 3). ¹H-NMR (400 MHz, CDCl₃) δ 7.90 (s, 1H), 2.70 (s, 3H), 2.67-2.58 (m, 2H), 2.19-2.10 (m, 3H), 1.77-1.70 (m, 1H), 1.56 (s, 3H), 1.35 (s, 12H).

### Intermediate B-62

### 5-chloro-2-methyl-6-(3-methyl-1-bicyclo[1.1.1]pentanyl)-3-(4,4,5,5-tetramethyl-1,3,2-dioxaborolan-2-yl)pyridine

### Step 1: 5-chloro-2-methyl-6-(3-methyl-1-bicyclo[1.1.1]pentanyl)-3-(4,4,5,5-tetramethyl-1,3,2-dioxaborolan-2-yl)pyridine (Intermediate B-62)

5-chloro-2-methyl-6-(3-methyl-1-bicyclo[1.1.1]pentanyl)-3-(4,4,5,5-tetramethyl-1,3,2-dioxaborolan-2-yl)pyridine (Intermediate B-62, 518 mg, 23%) was prepared from 3-methylbicyclo[1.1.1]pentane-1-carboxylic acid, using procedure analogous to that found in Intermediate B-53 (Step 1 to Step 3). as a yellow solid. ESI-MS m/z calc. 333.17, found 252.2 (M−81)⁺ ¹H NMR (400 MHz, CDCl₃) δ 7.87 (s, 1H), 2.68 (s, 3H), 2.16 (s, 6H), 1.34 (s, 12H), 1.26-1.24 (m, 3H).

### Intermediate B-63

### 3-chloro-2-(2,2-dimethylpyrrolidin-1-yl)-6-methyl-5-(4,4,5,5-tetramethyl-1,3,2-dioxaborolan-2-yl)pyridine

### Step 1: 3-bromo-6-(2,2-dimethylpyrrolidin-1-yl)-2-methyl-pyridine

To a solution of 3-bromo-6-fluoro-2-methyl-pyridine (1 g, 5.26 mmol) and 2,2-dimethylpyrrolidine (575 mg, 5.8 mmol) in DMSO (8 mL), was added K₂CO₃ (1.5 g, 10.85 mmol) in one portion. The reaction mixture was sealed under nitrogen and heated at 165° C. for 17 hours, cooled to room temperature and partitioned between water (40 mL) and Et₂O. The layers were separated and the aqueous phase extracted with Et₂O. The combined organic layer was washed with brine, dried over magnesium sulphate, filtered and concentrated under reduced pressure. Purification by silica gel flash chromatography using 100% hexanes provided 3-bromo-6-(2,2-dimethylpyrrolidin-1-yl)-2-methyl-pyridine (1 g, 71%). ESI-MS m/z calc. 268.06, found 269.0 (M+1)⁺. ¹H NMR (400 MHz, CDCl₃) δ 7.40 (d, J=8.8 Hz, 1H), 6.05 (d, J=8.8 Hz, 1H), 3.36 (t, J=6.3 Hz, 2H), 2.47 (s, 3H), 1.94-1.84 (m, 4H), 1.51 (s, 6H).

### Step 2: 5-bromo-3-chloro-2-(2,2-dimethylpyrrolidin-1-yl)-6-methyl-pyridine

To a stirring solution of 3-bromo-6-(2,2-dimethylpyrrolidin-1-yl)-2-methyl-pyridine (1.01 g, 3.75 mmol) in anhydrous DMF (2 mL), was added N-chlorosuccinimide (550 mg, 4.12 mmol) in one portion. The reaction was stirred at 50° C. for 1 hour, cooled to room temperature, diluted with water and extracted with Et₂O. The combined organic layer was washed with brine, dried over MgSO₄, filtered and concentrated under reduced pressure. Purification by silica gel flash chromatography using 100% hexanes provided 5-bromo-3-chloro-2-(2,2-dimethylpyrrolidin-1-yl)-6-methyl-pyridine (1.07 g, 94%). ESI-MS m/z calc. 302.02, found 303.0 (M+1)⁺. ¹H NMR (400 MHz, CDCl₃) δ 7.50 (s, 1H), 3.82 (t, J=6.9 Hz, 2H), 2.44 (s, 3H), 1.90 (p, J=7.0 Hz, 2H), 1.78 (t, J=6.9 Hz, 2H), 1.52 (s, 6H).

### Step 3: 3-chloro-2-(2,2-dimethylpyrrolidin-1-yl)-6-methyl-5-(4,4,5,5-tetramethyl-1,3,2-dioxaborolan-2-yl)pyridine (Intermediate B-63)

To a solution of 5-bromo-3-chloro-2-(2,2-dimethylpyrrolidin-1-yl)-6-methyl-pyridine (250 mg, 0.82 mmol) in anhydrous THF (7.5 mL) at −78° C. was added n-BuLi in cyclohexane (0.46 mL of 2 M, 0.92 mmol). The reaction mixture was stirred for 10 minutes before the addition of 2-isopropoxy-4,4,5,5-tetramethyl-1,3,2-dioxaborolane (0.17 mL, 0.83 mmol). The reaction mixture was warmed to room temperature, stirred for one hour and concentrated under reduced pressure to provide crude 3-chloro-2-(2,2-dimethylpyrrolidin-1-yl)-6-methyl-5-(4,4,5,5-tetramethyl-1,3,2-dioxaborolan-2-yl)pyridine (Intermediate B-63). ESI-MS m/z calc. 350.19, found 351.2 (M+1)⁺.

### Intermediate B-64

### methyl 2-[3-chloro-6-methyl-5-(4,4,5,5-tetramethyl-1,3,2-dioxaborolan-2-yl)-2-pyridyl]-2-methyl-propanoate

### Step 2: methyl 2-[3-chloro-6-methyl-5-(4,4,5,5-tetramethyl-1,3,2-dioxaborolan-2-yl)-2-pyridyl]-2-methyl-propanoate (Intermediate B-64)

Methyl 2-[3-chloro-6-methyl-5-(4,4,5,5-tetramethyl-1,3,2-dioxaborolan-2-yl)-2-pyridyl]-2-methyl-propanoate (Intermediate B-64, 135 mg, 74%) was prepared from methyl 2-(5-bromo-3-chloro-6-methyl-2-pyridyl)-2-methyl-propanoate using procedure analogous to that found in Intermediate B-1, Step 2. ESI-MS m/z calc. 353.15 found 354.2 (M+1)⁺. ¹H NMR (400 MHz, CDCl₃) δ 7.93 (s, 1H), 3.67 (s, 3H), 2.70 (s, 3H), 1.62 (s, 6H), 1.35 (s, 12H).

### Intermediate B-65

### [2-chloro-5-methyl-4-(4,4,5,5-tetramethyl-1,3,2-dioxaborolan-2-yl)phenyl]-trimethyl-silane

### Step 1: (4-bromo-2-chloro-5-methyl-phenyl)-trimethyl-silane

A solution of 1,4-dibromo-2-chloro-5-methyl-benzene (5 g, 17.58 mmol) in dry THF (100 mL) under an atmosphere of argon was cooled to −78° C. nBuLi in hexanes (11 mL of 1.6 M, 17.6 mmol) was added drop wise, then the reaction was stirred for a further 1 h before the dropwise addition of TMSCl (1.95 g, 17.95 mmol) in dry THF (25 mL) and the reaction mixture was stirred for 2 h. The reaction mixture was warmed slowly to room temperature and stirred for 16 h. The reaction mixture was quenched with saturated ammonium chloride (20 mL). and was extracted with EtOAc (2×20 mL), dried over sodium sulfate and concentrated under reduced pressure. Purification by reverse phase chromatography using 50-95% MeCN in water (0.1% v/v ammonia) as modifier gave (4-bromo-2-chloro-5-methyl-phenyl)-trimethyl-silane (1.4 g, 28%) as a yellow oil, ¹H-NMR (400 MHz, CDCl₃) δ 7.54 (s, 1H), 7.29 (s, 1H), 2.39 (s, 3H), 0.38 (s, 9H)

### Step 2: [2-chloro-5-methyl-4-(4,4,5,5-tetramethyl-1,3,2-dioxaborolan-2-yl)phenyl]-trimethyl-silane (Intermediate B-65)

[2-chloro-5-methyl-4-(4,4,5,5-tetramethyl-1,3,2-dioxaborolan-2-yl)phenyl]-trimethyl-silane (Intermediate B-65) was prepared from (4-bromo-2-chloro-5-methyl-phenyl)-trimethyl-silane using procedure analogous to that found in Intermediate B-1, Step 2. ¹H-NMR (400 MHz, CDCl₃) δ 7.68 (s, 1H), 7.21 (s, 1H), 2.48 (s, 3H), 1.33 (s, 12H), 0.35 (t, J=3.4 Hz, 9H).

### Intermediate B-66

### 2-[5-chloro-2-methyl-4-(1-methylcyclopropyl)phenyl]-4,4,5,5-tetramethyl-1,3,2-dioxaborolane

### Step 1: 2-(4-bromo-2-chloro-5-methyl-phenyl)propan-2-ol

To a solution of 1,4-dibromo-2-chloro-5-methyl-benzene (504 mg, 1.68 mmol) in diethyl ether (13 mL) at −78° C. was added n-BuLi (in hexanes) (0.7 mL of 2.5 M, 1.75 mmol). The reaction mixture was stirred at −78° C. for 1 h. Acetone (316.40 mg, 0.4 mL, 5.45 mmol) was added dropwise, and it was stirred for 90 minutes at −78° C. The dry-ice bath was removed, and the reaction was immediately quenched by slow addition of saturated aqueous ammonium chloride solution (20 mL). The biphasic mixture was poured in a separatory funnel with water (10 mL). The layers were separated and the aqueous layer was extracted with MTBE (2×15 mL). The combined organic layers were dried over anhydrous sodium sulfate, filtered and concentrated under reduced pressure. The residue was purified by flash chromatography on silica gel using 0 to 10% ethyl acetate in heptane to give 2-(4-bromo-2-chloro-5-methyl-phenyl)propan-2-ol (367 mg, 77%). ESI-MS m/z calc. 261.97, found 245.1 (M−17)⁺.

### Step 2: 1-bromo-5-chloro-4-isopropenyl-2-methyl-benzene

To a solution of 2-(4-bromo-2-chloro-5-methyl-phenyl)propan-2-ol (2.4 g, 7.84 mmol) in toluene (24 mL) was added p-toluenesulfonic acid monohydrate (150 mg, 0.79 mmol) and the reaction mixture was refluxed using Dean-stark apparatus for 16 h. After cooling it to room temperature, the mixture was diluted with MTBE (90 mL) and washed with saturated aqueous solution of sodium bicarbonate (30 mL). It was then washed with brine (30 mL), dried over anhydrous sodium sulfate, filtered and concentrated under reduced pressure to obtain crude 1-bromo-5-chloro-4-isopropenyl-2-methyl-benzene (2.1 g, 97%) as a yellow solid that was directly used in the next step without further purification. ESI-MS m/z calc. 243.96, found 245.0 (M+1)⁺. ¹H NMR (400 MHz, CDCl₃) δ 7.54 (s, 1H), 7.08 (s, 1H), 5.25-5.22 (m, 1H), 4.98-4.95 (m, 1H), 2.36 (s, 3H), 2.08 (s, 3H).

### Step 3: 1-bromo-5-chloro-2-methyl-4-(1-methylcyclopropyl)benzene

1-bromo-5-chloro-2-methyl-4-(1-methylcyclopropyl)benzene was prepared from 1-bromo-5-chloro-4-isopropenyl-2-methyl-benzene using procedure analogous to that found in Intermediate B-11, Step 2. ¹H NMR (400 MHz, CDCl₃) δ 7.50 (s, 1H), 7.21 (s, 1H), 2.34 (s, 3H), 1.32 (s, 3H), 0.80-0.74 (m, 4H).

### Step 4: 2-[5-chloro-2-methyl-4-(1-methylcyclopropyl)phenyl]-4,4,5,5-tetramethyl-1,3,2-dioxaborolane (Intermediate B-66)

2-[5-chloro-2-methyl-4-(1-methylcyclopropyl)phenyl]-4,4,5,5-tetramethyl-1,3,2-dioxaborolane (Intermediate B-66) was prepared from 1-bromo-5-chloro-2-methyl-4-(1-methylcyclopropyl)benzene using procedure analogous to that found in Intermediate B-1, Step 2. ESI-MS m/z calc. 306.15, found 307.1 (M+1)⁺. ¹H NMR (400 MHz, CDCl₃) δ 7.71 (s, 1H), 7.15 (s, 1H), 2.48 (s, 3H), 1.34-1.33 (m, 15H), 0.81-0.79 (m, 2H), 0.76-0.73 (m, 2H). (s, 3H).

### Intermediate B-67

### 2-[5-chloro-2-methyl-4-[1-(trifluoromethyl)cyclopropyl]phenyl]-4,4,5,5-tetramethyl-1,3,2-dioxaborolane

### Step 1: 1-(4-bromo-2-chloro-5-methyl-phenyl)-2,2,2-trifluoro-ethanone

To a stirred solution of 1,4-dibromo-2-chloro-5-methyl-benzene (65 g, 228.57 mmol) in diethyl ether (1.3 L) was added n-BuLi solution in hexanes (92 mL of 2.5 M, 230 mmol) at −78° C. The reaction mixture was kept at this temperature for 40 minutes then a solution of methyl trifluoroacetate (29.3 g, 23 mL, 228.65 mmol) in diethyl ether (160 mL) was added dropwise. The reaction mixture was stirred at this temperature for 40 minutes. A mixture of concentrated hydrochloric acid and ethanol (300 ml, 3/2) was added dropwise to quench the reaction at −78° C. The aqueous phase was extracted with methyl tert-butyl ether (3×). The combined organic layer was washed with water (200 mL), saturated aqueous solution of sodium chloride (300 mL), dried over sodium sulfate, filtered and concentrated under reduced pressure. The crude product was purified by silica gel column chromatography using 0 to 10% ethyl acetate in heptanes to provide 1-(4-bromo-2-chloro-5-methyl-phenyl)-2,2,2-trifluoro-ethanone (60.05 g, 78%). ESI-MS m/z calc. 299.91, found 301.0 (M+1)⁺. ¹H NMR (400 MHz, CDCl₃) δ 7.76 (s, 1H), 7.57 (s, 1H), 2.46 (s, 3H). ¹⁹F NMR (377 MHz, CDCl₃) δ −72.81 (s, 3F).

### Step 2: 1-bromo-5-chloro-2-methyl-4-[1-(trifluoromethyl)vinyl]benzene

To a solution of methyltriphenylphosphonium bromide (740 mg, 2.07 mmol) in THF (6.5 mL) at 0° C. was added dropwise nBuLi (in hexanes) (0.8 mL of 2.5 M, 2 mmol). After 20 min at 0° C. the reaction mixture was cooled to −78° C. and a solution of 1-(4-bromo-2-chloro-5-methyl-phenyl)-2,2,2-trifluoro-ethanone (522 mg, 1.45 mmol) in THF (1.5 mL) was added dropwise. The resulting mixture was stirred for 1 h at −78° C. then slowly warmed to rt for 65 h. The reaction mixture was quenched with saturated aqueous ammonium chloride solution (50 mL), extracted with MTBE (150 mL), dried over anhydrous sodium sulfate, filtered and concentrated under vacuum. The residue was filtered through a pad of silica gel with heptane and concentrated in-vacuo. The obtained residue was purified by reverse phase flash chromatography (C₁₈) using 5 to 98% MeCN in water (0.1% formic acid) to give 1-bromo-5-chloro-2-methyl-4-[1-(trifluoromethyl)vinyl]benzene (228 mg, 51%) as a colorless oil. ¹H NMR (400 MHz, CDCl₃) δ 7.65 (s, 1H), 7.16 (s, 1H), 6.21-6.19 (m, 1H), 5.64-5.61 (m, 1H), 2.38 (s, 3H).

### Step 3: 1-bromo-5-chloro-2-methyl-4-[1-(trifluoromethyl)cyclopropyl]benzene

A solution of 1-bromo-5-chloro-2-methyl-4-[1-(trifluoromethyl)vinyl]benzene (14.2 g, 47.41 mmol) and methyl(diphenyl)sulfonium;tetrafluoroborate (20 g, 69.42 mmol) in THF (150 mL) was cooled to −78° C. and a solution of LHMDS in THF (140 mL of 1 M, 140 mmol) was added dropwise using an addition funnel while maintaining the internal temperature below −65° C. The reaction mixture was stirred at −70° C. under an atmosphere of nitrogen and gradually warmed to room temperature overnight. The reaction mixture was quenched with saturated. ammonium chloride solution and extracted with EtOAc. The combined organic layer was dried over magnesium sulfate, filtered and concentrated. Purified via silica gel column chromatography using hexanes to obtain 1-bromo-5-chloro-2-methyl-4-[1-(trifluoromethyl)cyclopropyl]benzene (10.1 g, 68%). ¹H NMR (400 MHz, CDCl₃) δ 7.58 (s, 1H), 7.33 (s, 1H), 2.36 (s, 3H), 1.50-1.45 (m, 2H), 1.10-1.05 (m, 2H).

### Step 4: 2-[5-chloro-2-methyl-4-[1-(trifluoromethyl)cyclopropyl]phenyl]-4,4,5,5-tetramethyl-1,3,2-dioxaborolane (Intermediate B-67)

2-[5-chloro-2-methyl-4-[1-(trifluoromethyl)cyclopropyl]phenyl]-4,4,5,5-tetramethyl-1,3,2-dioxaborolane (Intermediate B-67) was prepared from 1-bromo-5-chloro-2-methyl-4-[1-(trifluoromethyl)cyclopropyl]benzene using procedure analogous to that found in Intermediate B-1, Step 2 as a pale yellow solid. ESI-MS m/z calc. 360.12, found 361.3 (M+1)⁺. ¹H NMR (400 MHz, CDCl₃) δ 7.76 (s, 1H), 7.27 (s, 1H), 2.49 (s, 3H), 1.48-1.44 (m, 2H), 1.33 (s, 12H), 1.08 (s, 2H).

### Intermediate B-68

### 2-[5-chloro-4-(3,3-difluoro-1-methyl-cyclobutyl)-2-methyl-phenyl]-4,4,5,5-tetramethyl-1,3,2-dioxaborolane

### Step 1: 2-[5-chloro-4-(3,3-difluoro-1-methyl-cyclobutyl)-2-methyl-phenyl]-4,4,5,5-tetramethyl-1,3,2-dioxaborolane (Intermediate B-68)

2-[5-chloro-4-(3,3-difluoro-1-methyl-cyclobutyl)-2-methyl-phenyl]-4,4,5,5-tetramethyl-1,3,2-dioxaborolane (Intermediate B-68) was prepared from 1-bromo-5-chloro-4-isopropenyl-2-methyl-benzene using procedure analogous to that found in Intermediate B-6 (Step 2 to Step 4). ESI-MS m/z calc. 356.15, found 357.2 (M+1)⁺. ¹H NMR (400 MHz, CDCl₃) δ 7.71 (s, 1H), 6.90 (s, 1H), 3.03-2.90 (m, 2H), 2.85-2.75 (m, 2H), 2.49 (s, 3H), 1.33 (s, 12H), 1.54 (s, 3H).

### Intermediate B-69

### 2-[5-chloro-2-methyl-4-[2-(trifluoromethyl)oxetan-2-yl]phenyl]-4,4,5,5-tetramethyl-1,3,2-dioxaborolane

### Step 1: 2-(4-bromo-2-chloro-5-methyl-phenyl)-2-(trifluoromethyl)oxetane

To a suspension of t-BuOK (215 mg, 1.92 mmol) in DMSO (2.5 mL) was added trimethylsulfoxonium iodide (417 mg, 1.9 mmol) and the reaction mixture was stirred at room temperature for 10 minutes. A solution of 1-(4-bromo-2-chloro-5-methyl-phenyl)-2,2,2-trifluoro-ethanone (200 mg, 0.63 mmol) in DMSO (0.6 mL) was then added dropwise to the above reaction mixture and it was stirred at room temperature for 16 h. The mixture was diluted with MTBE (60 mL) and brine (30 mL) and the layers were separated. The aqueous layer was extracted with MTBE (60 mL). The combined organic layer was washed with brine (30 mL), dried over anhydrous sodium sulfate, filtered and concentrated under reduced pressure. The residue was purified by silica gel chromatography using 0 to 20% EtOAc in heptane to afford 2-(4-bromo-2-chloro-5-methyl-phenyl)-2-(trifluoromethyl)oxetane (138 mg, 62%) as yellow oil. ¹H NMR (400 MHz, CDCl₃) δ 7.58 (s, 1H), 7.44 (s, 1H), 4.86 (td, J=7.9, 5.6 Hz, 1H), 4.54 (dt, J=8.4, 6.2 Hz, 1H), 3.32-3.22 (m, 2H), 2.41 (s, 3H). ¹⁹F NMR (377 MHz, CDCl₃) δ −82.05 (s, 3F).

### Step 2: 2-[5-chloro-2-methyl-4-[2-(trifluoromethyl)oxetan-2-yl]phenyl]-4,4,5,5-tetramethyl-1,3,2-dioxaborolane (Intermediate B-69)

2-[5-chloro-2-methyl-4-[2-(trifluoromethyl)oxetan-2-yl]phenyl]-4,4,5,5-tetramethyl-1,3,2-dioxaborolane (Intermediate B-69) was prepared from 2-(4-bromo-2-chloro-5-methyl-phenyl)-2-(trifluoromethyl)oxetane using procedure analogous to that found in Intermediate B-1, Step 2 as a white solid. ESI-MS m/z calc. 376.12, found 377.2 (M+1)⁺.

### Intermediate B-70

### 2-[5-chloro-4-(2,2-difluorospiro[3.3]heptan-6-yl)-2-methyl-phenyl]-4,4,5,5-tetramethyl-1,3,2-dioxaborolane

### Step 1: 6-(4-bromo-2-chloro-5-methyl-phenyl)-2,2-difluoro-spiro[3.3]heptan-6-ol

To a solution of 1-bromo-5-chloro-4-iodo-2-methyl-benzene (1 g, 3.02 mmol) in diethyl ether (20 mL) at −78° C., was added n-BuLi (1.63 mL of 2 M, 3.26 mmol) dropwise. The reaction mixture was stirred for 5 min before the addition of 2,2-difluorospiro[3.3]heptan-6-one (500 mg, 3.25 mmol). The reaction mixture was warmed to room temperature and stirred for 2.5 h. The reaction mixture was quenched with 1M HCl and the layers were separated. The organic layer was washed with saturated aqueous sodium bicarbonate solution, followed by washing with brine, dried over magnesium sulfate, filtered and concentrated under reduced pressure. Purification by silica gel column chromatography using 0 to 60% EtOAc in hexanes provided 6-(4-bromo-2-chloro-5-methyl-phenyl)-2,2-difluoro-spiro[3.3]heptan-6-ol (842 mg, 79%). ¹H NMR (400 MHz, CDCl₃) δ 7.55 (s, 1H), 7.14 (s, 1H), 2.88-2.73 (m, 4H), 2.66-2.59 (m, 2H), 2.53-2.45 (m, 2H), 2.37 (s, 3H).

### Step 2: 6-(4-bromo-2-chloro-5-methyl-phenyl)-2,2-difluoro-spiro[3.3]heptane

To a solution of 6-(4-bromo-2-chloro-5-methyl-phenyl)-2,2-difluoro-spiro[3.3]heptan-6-ol (100 mg, 0.28 mmol) and triethylsilane (0.1 mL, 0.62 mmol) in DCM (1 mL) at −78° C. was added diethyloxonio(trifluoro)boranuide (0.1 mL, 0.81 mmol) dropwise. The reaction mixture was warmed to room temperature and stirred for 1.5 h. It was diluted with EtOAc and washed with saturated aqueous sodium bicarbonate, dried over magnesium sulfate, filtered and concentrated under reduced pressure. Purification by silica gel column chromatography using 0 to 10% EtOAc in hexanes provided 6-(4-bromo-2-chloro-5-methyl-phenyl)-2,2-difluoro-spiro[3.3]heptane (73 mg, 76%). ¹H NMR (400 MHz, CDCl₃) δ 7.49 (s, 1H), 7.06 (s, 1H), 3.61 (p, J=8.6 Hz, 1H), 2.85-2.69 (m, 2H), 2.60-2.46 (m, 4H), 2.36 (s, 3H), 2.28-2.17 (m, 2H).

### Step 3: 2-[5-chloro-4-(2,2-difluorospiro[3.3]heptan-6-yl)-2-methyl-phenyl]-4,4,5,5-tetramethyl-1,3,2-dioxaborolane (Intermediate B-70)

2-[5-chloro-4-(2,2-difluorospiro[3.3]heptan-6-yl)-2-methyl-phenyl]-4,4,5,5-tetramethyl-1,3,2-dioxaborolane (Intermediate B-70) was prepared from 6-(4-bromo-2-chloro-5-methyl-phenyl)-2,2-difluoro-spiro[3.3]heptane using procedure analogous to that found in Intermediate B-1, Step 2.1H NMR (400 MHz, CDCl₃) δ 7.68 (s, 1H), 7.00 (s, 1H), 3.68 (p, J=8.9 Hz, 1H), 2.74 (t, J=12.5 Hz, 2H), 2.59-2.45 (m, 7H), 2.25 (td, J=9.6, 2.9 Hz, 2H), 1.33 (s, 12H).

### Intermediate B-71

### 2-[4-(1-bicyclo[3.1.0]hexanyl)-5-chloro-2-methyl-phenyl]-4,4,5,5-tetramethyl-1,3,2-dioxaborolane

### Step 1: 1-(4-bromo-2-chloro-5-methyl-phenyl)cyclopentanol

1-(4-bromo-2-chloro-5-methyl-phenyl)cyclopentanol prepared using procedure analogous to Intermediated B-73, Step 1, using cyclopentanone. ¹H NMR (400 MHz, CDCl₃) δ 7.54 (s, 1H), 7.48 (s, 1H), 2.37 (s, 3H), 2.30-2.19 (m, 3H), 2.08-2.02 (m, 2H), 2.00-1.90 (m, 2H), 1.87-1.77 (m, 2H).

### Step 2: 1-bromo-5-chloro-4-(cyclopenten-1-yl)-2-methyl-benzene

To a solution of 1-(4-bromo-2-chloro-5-methyl-phenyl)cyclopentanol (465 mg, 1.6 mmol) and triethylamine (455 μL, 3.26 mmol) in dichloromethane (10 mL) at 0° C. was added methanesulfonyl chloride (250 μL, 3.23 mmol). The reaction mixture was stirred at room temperature for 22 h. The reaction mixture was diluted with dichloromethane (30 mL). The organic layer was washed with water (30 mL) and saturated aqueous sodium bicarbonate (30 mL) then dried over anhydrous sodium sulfate, filtered and concentrated under reduced pressure. Purification by silica gel chromatography using 0 to 30% EtOAc in hexanes provided 1-bromo-5-chloro-4-(cyclopenten-1-yl)-2-methyl-benzene (236 mg, 54%). ESI-MS m/z calc. 269.98, found 271.0 (M+1)⁺. ¹H NMR (400 MHz, CDCl₃) δ 7.53 (s, 1H), 7.13 (s, 1H), 6.16-6.05 (m, 1H), 2.76-2.66 (m, 2H), 2.57-2.47 (m, 2H), 2.34 (s, 3H), 1.99 (p, J=7.5 Hz, 2H).

### Step 3: 1-(4-bromo-2-chloro-5-methyl-phenyl)bicyclo[3.1.0]hexane

A solution of 1-bromo-5-chloro-4-(cyclopenten-1-yl)-2-methyl-benzene (100 mg, 0.37 mmol) in DCM (1 mL) was treated with diethylzinc (1.5 mL of 15% w/v, 1.82 mmol). After 10 min stirring at room temperature, the mixture was cooled to 0° C. (ice bath) and treated with a solution of diiodomethane (150 μL, 1.86 mmol) in DCM (300 μL) dropwise over 4 min. The reaction mixture was stirred at room temperature for 18 h then quenched with sat. ammonium chloride. The mixture was extracted with DCM, dried over sodium sulfate, filtered and concentrated. Purification by silica gel chromatography eluting with a gradient system of 0 to 20% EtOAc in hexanes, followed by a second purification using reverse phase chromatography using 1 to 99% MeCN in water (HCl modifier) gave 1-(4-bromo-2-chloro-5-methyl-phenyl)bicyclo[3.1.0]hexane (43 mg, 41%). ¹H NMR (400 MHz, CDCl₃) δ 7.49 (s, 1H), 7.18 (s, 1H), 2.33 (s, 3H), 2.11-2.02 (m, 1H), 1.98-1.88 (m, 1H), 1.89-1.77 (m, 2H), 1.76-1.65 (m, 1H), 1.52-1.45 (m, 1H), 1.37-1.21 (m, 1H), 0.82 (t, J=4.6 Hz, 1H), 0.67 (dd, J=8.4, 5.0 Hz, 1H).

### Step 4: 2-[4-(1-bicyclo[3.1.0]hexanyl)-5-chloro-2-methyl-phenyl]-4,4,5,5-tetramethyl-1,3,2-dioxaborolane (Intermediate B-71)

2-[4-(1-bicyclo[3.1.0]hexanyl)-5-chloro-2-methyl-phenyl]-4,4,5,5-tetramethyl-1,3,2-dioxaborolane (Intermediate B-71) was prepared from 1-(4-bromo-2-chloro-5-methyl-phenyl)bicyclo[3.1.0]hexane using procedure analogous to that found in Intermediate B-1, Step 2. ESI-MS m/z calc. 332.17, found 333.2 (M+1)⁺. ¹H NMR (400 MHz, CDCl₃) δ 7.70 (s, 1H), 7.13 (s, 1H), 2.46 (s, 3H), 2.14-2.01 (m, 1H), 1.99-1.86 (m, 2H), 1.86-1.76 (m, 1H), 1.76-1.66 (m, 1H), 1.53-1.45 (m, 1H), 1.32 (s, 12H), 0.93-0.82 (m, 1H), 0.81 (m, 1H), 0.74-0.63 (m, 1H).

### Intermediate B-72

### 2-(5-chloro-4-cyclobutyl-2-methyl-phenyl)-4,4,5,5-tetramethyl-1,3,2-dioxaborolane

### Step 1: 2-(5-chloro-4-cyclobutyl-2-methyl-phenyl)-4,4,5,5-tetramethyl-1,3,2-dioxaborolane (Intermediate B-72)

2-(5-chloro-4-cyclobutyl-2-methyl-phenyl)-4,4,5,5-tetramethyl-1,3,2-dioxaborolane (Intermediate B-72) was prepared using procedure analogous to Intermediate B-70 (Step 1 to Step 3) using cyclobutanone. ESI-MS m/z calc. 306.16, found 307.2 (M+1)⁺. ¹H NMR (400 MHz, CDCl₃) δ 7.69 (s, 1H), 7.10 (s, 1H), 3.77 (quin, J=8.7 Hz, 1H), 2.52 (s, 3H), 2.45-2.35 (m, 2H), 2.19-1.98 (m, 3H), 1.88-1.78 (m, 1H), 1.34 (s, 12H).

### Intermediate B-73

### 2-(5-chloro-2-methyl-4-spiro[2.3]hexan-5-yl-phenyl)-4,4,5,5-tetramethyl-1,3,2-dioxaborolane

### Step 1: 2-(5-chloro-2-methyl-4-spiro[2.3]hexan-5-yl-phenyl)-4,4,5,5-tetramethyl-1,3,2-dioxaborolane Intermediate B-73)

2-(5-chloro-2-methyl-4-spiro[2.3]hexan-5-yl-phenyl)-4,4,5,5-tetramethyl-1,3,2-dioxaborolane (Intermediate B-73) was prepared using procedure analogous to Intermediate B-70 (Step 1 to Step 3) using spiro[2.3]hexan-5-one. ESI-MS m/z calc. 332.17, found 333.27 (M+1)⁺. ¹H NMR (400 MHz, CDCl₃) δ 7.70 (s, 1H), 7.20 (s, 1H), 4.05-3.94 (m, 1H), 2.52 (s, 3H), 2.43-2.35 (m, 4H), 1.33 (s, 12H), 0.63-0.52 (m, 2H), 0.45-0.38 (m, 2H).

### Intermediate B-74

### 2-[2-chloro-5-methyl-4-(4,4,5,5-tetramethyl-1,3,2-dioxaborolan-2-yl)phenyl]adamantan-2-ol

### Step 1: 2-[2-chloro-5-methyl-4-(4,4,5,5-tetramethyl-1,3,2-dioxaborolan-2-yl)phenyl]adamantan-2-ol (Intermediate B-74)

2-[2-chloro-5-methyl-4-(4,4,5,5-tetramethyl-1,3,2-dioxaborolan-2-yl)phenyl]adamantan-2-ol (Intermediate B-74) was prepared using procedure analogous to Intermediate B-70 (Step 1 and Step 3) using adamantan-2-one. ¹H NMR (400 MHz, CDCl₃) δ 7.72 (s, 1H), 7.35 (s, 1H), 2.50 (s, 3H), 1.90-1.61 (m, 13H), 1.33 (s, 12H).

### Intermediate B-75

### 2-[5-chloro-2-methyl-4-(1-methylcyclobutyl)phenyl]-4,4,5,5-tetramethyl-1,3,2-dioxaborolane

### Step 1: 1-(4-bromo-2-chloro-5-methyl-phenyl)cyclobutanol

A flask charged with 1,4-dibromo-2-chloro-5-methyl-benzene (92 g, 323.5 mmol) in THF (900 mL) under an atmosphere of nitrogen was cooled to −78° C. n-BuLi (in hexanes) (130 mL of 2.5 M, 325 mmol) was added slowly and the reaction mixture was stirred at −78° C. for 1 h and then, cyclobutanone (23.14 g, 24.67 mL, 323.5 mmol) was added. The reaction mixture was stirred at −78° C. for 3 h and then warmed to 0° C. and quenched by the addition of a saturated aqueous ammonium chloride solution (200 mL). The aqueous layer was extracted with EtOAc (2×250 mL). The combined organic layer was washed with brine, dried over sodium sulfate, filtered and concentrated under reduced pressure. The crude product was purified via silica gel column chromatography using 0 to 20% EtOAc in hexanes to afford 1-(4-bromo-2-chloro-5-methyl-phenyl)cyclobutanol (68.16 g, 76%) ESI-MS m/z calc. 273.97, found 257.027 (M−17)⁺ as a brown oil.

### Step 2: [1-(4-bromo-2-chloro-5-methyl-phenyl)cyclobutyl] methanesulfonate

To a solution of 1-(4-bromo-2-chloro-5-methyl-phenyl)cyclobutanol (3.43 g, 11.98 mmol) and triethylamine (2.47 g, 3.4 mL, 24.39 mmol) in dichloromethane (70 mL) at 0° C. was added methanesulfonyl chloride (2.51 g, 1.7 mL, 21.96 mmol). The reaction mixture was stirred at room temperature for 21 h and diluted with dichloromethane (150 mL). The organic layer was washed with water (150 mL), saturated aqueous sodium bicarbonate (150 mL), dried over anhydrous sodium sulfate, filtered and concentrated under reduced pressure to afford crude [1-(4-bromo-2-chloro-5-methyl-phenyl)cyclobutyl] methanesulfonate (4.24 g, 100%). ESI-MS m/z calc. 351.95, found 257.0 (M−97)⁺.

### Step 3: 1-bromo-5-chloro-2-methyl-4-(1-methylcyclobutyl)benzene

To a stirred solution of [1-(4-bromo-2-chloro-5-methyl-phenyl)cyclobutyl] methanesulfonate (4.24 g, 12 mmol) in dichloromethane (45 mL) at 0° C. and under nitrogen was added trimethylaluminum (in toluene) (21 mL of 2 M, 42. mmol). The reaction mixture was stirred at room temperature for 3 days and then, poured into a stirring mixture of dichloromethane (50 mL) and 1N aqueous HCl (50 mL). The layers were separated and the aqueous layer was extracted with dichloromethane (40 mL). The combined organic layers were washed with 1N aqueous HCl (50 mL) and brine (10 mL), dried over anhydrous sodium sulfate, filtered and evaporated under reduced pressure. The crude product was purified by silica gel column chromatography using heptanes to afford 1-bromo-5-chloro-2-methyl-4-(1-methylcyclobutyl)benzene (1.16 g, 26%) as a colorless oil. ¹H NMR (400 MHz, CDCl₃) δ 7.45 (s, 1H), 6.94 (s, 1H), 2.47-2.38 (m, 2H), 2.38-2.32 (m, 3H), 2.22-2.07 (m, 3H), 1.83-1.74 (m, 1H), 1.49 (s, 3H).

### Step 4: 2-[5-chloro-2-methyl-4-(1-methylcyclobutyl)phenyl]-4,4,5,5-tetramethyl-1,3,2-dioxaborolane (Intermediate B-75)

2-[5-chloro-2-methyl-4-(1-methylcyclobutyl)phenyl]-4,4,5,5-tetramethyl-1,3,2-dioxaborolane (Intermediate B-75) was prepared from 1-bromo-5-chloro-2-methyl-4-(1-methylcyclobutyl using procedure analogous to that found in Intermediate B-1, Step 2. ESI-MS m/z calc. 320.17, found 321.1 (M+1)⁺. ¹H NMR (400 MHz, CDCl₃) δ 7.66 (s, 1H), 6.89 (s, 1H), 2.52-2.39 (m, 5H), 2.22-2.05 (m, 3H), 1.81-1.70 (m, 1H), 1.50 (s, 3H), 1.33 (s, 12H).

### Intermediate B-76

### 2-[5-chloro-4-(2,2-difluoro-1,1-dimethyl-ethyl)-2-methyl-phenyl]-4,4,5,5-tetramethyl-1,3,2-dioxaborolane

### Step 1: 2-[5-chloro-4-(2,2-difluoro-1,1-dimethyl-ethyl)-2-methyl-phenyl]-4,4,5,5-tetramethyl-1,3,2-dioxaborolane (Intermediate B-76)

2-[5-chloro-4-(2,2-difluoro-1,1-dimethyl-ethyl)-2-methyl-phenyl]-4,4,5,5-tetramethyl-1,3,2-dioxaborolane (Intermediate B-76) was prepared using procedure analogous to Intermediate B-75 (Step1 to Step 4) using 1,1-difluoropropan-2-one. ESI-MS m/z calc. 344.15, found 345.187 (M+1)⁺.

### Intermediate B-77

### 2-[4-(3-bicyclo[3.1.0]hexanyl)-5-chloro-2-methyl-phenyl]-4,4,5,5-tetramethyl-1,3,2-dioxaborolane

### Step 1: 3-(4-bromo-2-chloro-5-methyl-phenyl)bicyclo[3.1.0]hexan-3-ol

3-(4-bromo-2-chloro-5-methyl-phenyl)bicyclo[3.1.0]hexan-3-ol prepared using procedure analogous to Intermediate B-70, Step1, using bicyclo[3.1.0]hexan-3-one. ¹H NMR (400 MHz, CDCl₃) δ 7.54 (s, 1H), 7.51 (s, 1H), 2.85-2.73 (m, 2H), 2.37 (s, 3H), 1.97 (d, J=14.2 Hz, 2H), 1.84 (s, 1H), 1.54-1.47 (m, 2H), 0.88 (q, J=4.0 Hz, 1H), 0.62-0.54 (m, 1H).

### Step 2: 3-(4-bromo-2-chloro-5-methyl-phenyl)bicyclo[3.1.0]hex-2-ene

To a solution of 3-(4-bromo-2-chloro-5-methyl-phenyl)bicyclo[3.1.0]hexan-3-ol (655 mg, 2.06 mmol) and triethylamine (747.78 mg, 1.03 mL, 7.39 mmol) in dichloromethane (16 mL) cooled to 0° C. in an ice/water bath was added methanesulfonyl chloride (666 mg, 0.45 mL, 5.81 mmol). After 5 minutes, the cold bath was removed and the reaction mixture was stirred at room temperature for 17 hours. The reaction mixture was partitioned between saturated aqueous sodium bicarbonate solution (60 mL) and dichloromethane (40 mL). The layers were separated and the aqueous layer was extracted again with dichloromethane (2×25 mL). The combined organic layer was dried over sodium sulfate, filtered and concentrated under reduced pressure. Purification by silica gel column chromatography using heptane afforded 3-(4-bromo-2-chloro-5-methyl-phenyl)bicyclo[3.1.0]hex-2-ene (493 mg, 82%) as a colorless oil. ESI-MS m/z calc. 281.98, found 283.0 (M+1)⁺. ¹H NMR (400 MHz, CDCl₃) δ 7.52 (s, 1H), 7.07 (s, 1H), 6.33 (q, J=2.0 Hz, 1H), 3.05 (ddd, J=17.1, 7.3, 1.6 Hz, 1H), 2.70 (br d, J=17.1 Hz, 1H), 2.33 (s, 3H), 2.00-1.91 (m, 1H), 1.75-1.65 (m, 1H), 0.93 (td, J=7.6, 3.8 Hz, 1H), 0.12 (q, J=3.8 Hz, 1H).

### Step 3: 3-(4-bromo-2-chloro-5-methyl-phenyl)bicyclo[3.1.0]hexane

A solution of 3-(4-bromo-2-chloro-5-methyl-phenyl)bicyclo[3.1.0]hex-2-ene (43 mg, 0.14 mmol) in ethyl acetate (2.5 mL) was sparged with nitrogen gas for about 5 minutes. Rhodium on alumina (20 mg, 5% w/w, 0.01 mmol) was added and the reaction mixture was sparged with hydrogen gas for 2-3 minutes and the reaction was stirred under an atmosphere of hydrogen gas for 4 hours. The reaction mixture was sparged under nitrogen atmosphere, then filtered over a short pad of celite and washed with ethyl acetate (10 mL). The solvent was removed under reduced pressure to afford crude 3-(4-bromo-2-chloro-5-methyl-phenyl)bicyclo[3.1.0]hexane (41 mg, 61%) as a colorless oil that was used directly in the next step without further purification.

### Step 4: 2-[4-(3-bicyclo[3.1.0]hexanyl)-5-chloro-2-methyl-phenyl]-4,4,5,5-tetramethyl-1,3,2-dioxaborolane (Intermediate B-77)

2-[4-(3-bicyclo[3.1.0]hexanyl)-5-chloro-2-methyl-phenyl]-4,4,5,5-tetramethyl-1,3,2-dioxaborolane (Intermediate B-77) was prepared from 3-(4-bromo-2-chloro-5-methyl-phenyl)bicyclo[3.1.0]hexane using procedure analogous to that found in Intermediate B-1, Step 2. ESI-MS m/z calc. 332.17, found 333.2 (M+1)⁺.

### Intermediate B-78

### 2-[5-chloro-2-methyl-4-(3-methyl-3-bicyclo[3.1.0]hexanyl)phenyl]-4,4,5,5-tetramethyl-1,3,2-dioxaborolane

### Step 1: 3-(4-bromo-2-chloro-5-methyl-phenyl)-3-methyl-bicyclo[3.1.0]hexane

N-Nitroso-N-methylurea (1.14 g, 11.06 mmol) was added to a biphasic mixture of KOH (40% w/w aqueous) (3 mL) and diethyl ether (10 mL) cooled to 0° C. The reaction mixture was stirred at 0° C. for 25 minutes (turns yellow), decanted and then cooled to −78° C. using a dry ice/acetone bath. Once the aqueous layer was frozen, the ether layer was added dropwise over 2-5 minutes to a solution of 1-bromo-5-chloro-2-methyl-4-(1-methylcyclopent-3-en-1-yl)benzene (105 mg, 0.36 mmol) and palladium (II) acetate (16.5 mg, 0.07 mmol) in dichloromethane (2 mL) at 0° C. After about 1.5 hour, the reaction mixture was filtered over a short pad of celite and washed with diethyl ether (15-20 mL). The volatiles were removed under reduced pressure to afford an orange oily residue. This residue was taken up in tert-butanol (1 mL) and water (1 mL). and methanesulfonamide (9.6 mg, 0.1 mmol) was added followed by AD-mix-α (233 mg) and the mixture was stirred vigorously at room temperature for 48 hours. A second portion of methanesulfonamide (10 mg, 0.1 mmol) and AD-mix-alpha (231 mg, 0.3 mmol) was added, and the reaction mixture was further diluted with tert-butanol (1 mL) and water (1 mL) and stirring was continued for another 70 hours. Added sodium sulfite (1.07 g) and stirring was continued for another 30 minutes. The reaction mixture was partitioned between water (30 mL) and MTBE (15 mL), the layers were separated and the aqueous layer was extracted with MTBE (15 mL). The combined organic layer was washed with water (15 mL), brine (15 mL), dried over sodium sulfate, filtered and concentrated under reduced pressure. Purification by silica gel column chromatography using 100% heptane afforded a 3.4:1 mixture of diastereomers of 3-(4-bromo-2-chloro-5-methyl-phenyl)-3-methyl-bicyclo[3.1.0]hexane (72 mg, 63%) as a colorless oil. ESI-MS m/z calc. 298.01, found 299.0 (M+1)⁺. ¹H NMR (400 MHz, CDCl₃) δ 7.52 (s, 1H), 7.18 (s, 1H), 2.57 (dd, J=13.7, 4.6 Hz, 2H), 2.36 (s, 3H), 1.91 (d, J=13.7 Hz, 2H), 1.48-1.40 (m, 2H), 1.34 (s, 3H), 0.63-0.56 (m, 1H), 0.48-0.42 (m, 1H).

### Step 2: 2-[5-chloro-2-methyl-4-(3-methyl-3-bicyclo[3.1.0]hexanyl)phenyl]-4,4,5,5-tetramethyl-1,3,2-dioxaborolane (Intermediate B-78)

2-[5-chloro-2-methyl-4-(3-methyl-3-bicyclo[3.1.0]hexanyl)phenyl]-4,4,5,5-tetramethyl-1,3,2-dioxaborolane (Intermediate B-78) was prepared from 3-(4-bromo-2-chloro-5-methyl-phenyl)-3-methyl-bicyclo[3.1.0]hexane using procedure analogous to that found in Intermediate B-1, Step 2. ESI-MS m/z calc. 346.1871, found 347.2 (M+1)⁺.

### Intermediate B-79

### 2-[4-(1-bicyclo[1.1.1]pentanyl)-5-chloro-2-methyl-phenyl]-4,4,5,5-tetramethyl-1,3,2-dioxaborolane

### Step 1: 1-(4-bromo-2-chloro-5-methyl-phenyl)bicyclo[1.1.1]pentane

A microwave vial was loaded with 1-bromo-5-chloro-4-iodo-2-methyl-benzene (500 mg, 1.51 mmol), [Ir(dtbbpy)(ppy)2]PF₆ (69 mg, 0.08 mmol), acetonitrile (3 mL), septa capped, and sparged with nitrogen (bubbling via a needle) for 10 minutes. To the reaction mixture was added tricyclo[1.1.1.0\hat{ }{1,3}]pentane in diethyl ether (2.9 mL) via syringe and the reaction mixture was sparged with nitrogen (bubbling via a needle) for 1 minute. The reaction mixture was irradiated with blue light (450 nM, 30 W) for 19 hours. The solution was directly purified by silica gel column chromatography using 100% Hexanes followed by a second purification using reverse phase preparative chromatography using a C₁₈ column and a gradient eluent of 1 to 99% acetonitrile in water containing 5 mM hydrochloric acid to give 1-(4-bromo-2-chloro-5-methyl-phenyl)-3-iodo-bicyclo[1.1.1]pentane (342 mg, 57%) as a clear oil. ¹H NMR (400 MHz, CDCl₃) δ 7.47 (s, 1H), 6.89 (s, 1H), 2.72 (s, 6H), 2.33 (s, 3H).

### Step 2: 1-(4-bromo-2-chloro-5-methyl-phenyl)bicyclo[1.1.1]pentane

A solution of 1-(4-bromo-2-chloro-5-methyl-phenyl)-3-iodo-bicyclo[1.1.1]pentane (5.19 g, 13.06 mmol) in THF (50 mL) in a heat-dried 500-mL flask under nitrogen was treated dropwise with a solution of lithium borohydride (in THF) (20 mL of 2 M, 40 mmol). The mixture was then stirred at room temperature for 2 h. InCl3 (3.40 g, 15.37 mmol) was added portion wise to minimize bubbling and mixture stirred for 24 h under nitrogen. The mixture was cooled to 0° C. (ice bath) and treated DROPWisE with water (120 mL) followed by the careful addition of aqueous NaOH (40 mL of 1 M, 40 mmol) followed by MTBE (200 mL). The layers were separated and the aqueous phase extracted with MTBE (3×50 mL). The combined organic layers were washed with water and brine, dried over sodium sulfate, filtered and concentrated in vacuo to provide 1-(4-bromo-2-chloro-5-methyl-phenyl)bicyclo[1.1.1]pentane (3.50 g, 99%) as a clear, colorless oil. ¹H NMR (400 MHz, CDCl₃) δ 7.45 (s, 1H), 6.98 (s, 1H), 2.56 (s, 1H), 2.33 (s, 3H), 2.22 (s, 6H).

### Step 3: 2-[4-(1-bicyclo[1.1.1]pentanyl)-5-chloro-2-methyl-phenyl]-4,4,5,5-tetramethyl-1,3,2-dioxaborolane (Intermediate B-79)

2-[4-(1-bicyclo[1.1.1]pentanyl)-5-chloro-2-methyl-phenyl]-4,4,5,5-tetramethyl-1,3,2-dioxaborolane (Intermediate B-79) was prepared from 1-(4-bromo-2-chloro-5-methyl-phenyl)bicyclo[1.1.1]pentane using procedure analogous to that found in Intermediate B-1, Step 2. ¹H NMR (400 MHz, CDCl₃) δ 7.64 (s, 1H), 6.93 (s, 1H), 2.55 (s, 1H), 2.46 (s, 3H), 2.23 (s, 6H), 1.32 (s, 12H).

### Intermediate B-80

### 2-[5-chloro-4-(4,4-difluoro-1-methyl-cyclohexyl)-2-methyl-phenyl]-4,4,5,5-tetramethyl-1,3,2-dioxaborolane

### Step 1: 5-chloro-4-(4,4-difluoro-1-methyl-cyclohexyl)-2-methyl-phenol

To a stirring solution of 5-chloro-2-methyl-phenol (0.48 g, 3.35 mmol) and 4,4-difluoro-1-methyl-cyclohexanol (504 mg, 3.35 mmol) in heptane (4 mL) at 5° C. was added sulfuric acid (200 μL, 3.75 mmol) dropwise. The mixture was allowed to warm to room temperature and stirred vigorously for 72 h under nitrogen. The mixture was poured into ice-water and the layers separated. The aqueous layer was extracted with ethyl acetate and the combined organic extracts were washed with brine (50 mL), dried over magnesium sulfate and concentrated. Purification by silica gel chromatography using 0-5% ethyl acetate/hexane provided 5-chloro-4-(4,4-difluoro-1-methyl-cyclohexyl)-2-methyl-phenol (454 mg, 49%). ESI-MS m/z calc. 274.09, found 275.13 (M+1)⁺. ¹H NMR (400 MHz, DMSO-d₆) δ 9.62 (s, 1H), 7.16 (s, 1H), 6.81 (s, 1H), 2.41-2.32 (m, 2H), 2.10 (s, 3H), 2.09-1.92 (m, 2H), 1.88-1.73 (m, 4H), 1.34 (s, 3H).

### Step 2: 2-[5-chloro-4-(4,4-difluoro-1-methyl-cyclohexyl)-2-methyl-phenyl]-4,4,5,5-tetramethyl-1,3,2-dioxaborolane (Intermediate B-80)

2-[5-chloro-4-(4,4-difluoro-1-methyl-cyclohexyl)-2-methyl-phenyl]-4,4,5,5-tetramethyl-1,3,2-dioxaborolane (Intermediate B-80) was prepared from 5-chloro-4-(4,4-difluoro-1-methyl-cyclohexyl)-2-methyl-phenol using procedure analogous to Intermediate B-26 (Step 4 and Step 5). ESI-MS m/z calc. 384.18, found 385.3 (M+1)⁺. ¹H NMR (400 MHz, DMSO-d₆) δ 7.55 (s, 1H), 7.31 (s, 1H), 2.45 (s, 3H), 2.44-2.37 (m, 2H), 2.10-1.95 (m, 2H), 1.94-1.83 (m, 2H), 1.83-1.70 (m, 2H), 1.38 (s, 3H), 1.29 (s, 12H). ¹⁹F NMR (376 MHz, DMSO-d₆) δ −92.98-95.81 (m).

### Intermediate B-81

### 2-[5-chloro-2-methyl-4-(1-methylcyclopentyl)phenyl]-4,4,5,5-tetramethyl-1,3,2-dioxaborolane

### Step 1: 2-[5-chloro-2-methyl-4-(1-methylcyclopentyl)phenyl]-4,4,5,5-tetramethyl-1,3,2-dioxaborolane (Intermediate B-81)

2-[5-chloro-2-methyl-4-(1-methylcyclopentyl)phenyl]-4,4,5,5-tetramethyl-1,3,2-dioxaborolane (Intermediate B-81) was prepared using procedure analogous to Intermediate B-80 (Step 1 and Step 2) by using 1-methylcyclopentanol. ¹H NMR (400 MHz, DMSO-d₆) δ 7.52 (s, 1H), 7.22 (s, 1H), 2.42 (s, 3H), 2.12-1.99 (m, 2H), 1.96-1.81 (m, 2H), 1.80-1.57 (m, 4H), 1.29 (s, 12H), 1.27 (s, 3H).

### Intermediate B-82

### 2-[5-fluoro-2-methyl-4-(1-methylcyclopentyl)phenyl]-4,4,5,5-tetramethyl-1,3,2-dioxaborolane

### Step 1: 2-[5-fluoro-2-methyl-4-(1-methylcyclopentyl)phenyl]-4,4,5,5-tetramethyl-1,3,2-dioxaborolane (Intermediate B-82)

2-[5-fluoro-2-methyl-4-(1-methylcyclopentyl)phenyl]-4,4,5,5-tetramethyl-1,3,2-dioxaborolane (Intermediate B-82) was prepared using procedure analogous to Intermediate B-80 (Step 1 and Step 2) by using 5-fluoro-2-methyl-phenol and 1-methylcyclopentanol instead of 5-choro-2-methyl-phenol and 4,4-difluoro-1-methyl-cyclohexanol respectively. ¹H NMR (400 MHz, CDCl₃) δ 7.37 (d, J=12.5 Hz, 1H), 7.05 (d, J=7.6 Hz, 1H), 2.48 (s, 3H), 1.94-1.85 (m, 4H), 1.82-1.66 (m, 4H), 1.32 (s, 12H), 1.24 (s, 3H).

### Intermediate B-83

### 2-(5-chloro-4-cyclopentyl-2-methyl-phenyl)-4,4,5,5-tetramethyl-1,3,2-dioxaborolane

### Step 1: 2-(5-chloro-4-cyclopentyl-2-methyl-phenyl)-4,4,5,5-tetramethyl-1,3,2-dioxaborolane (Intermediate B-83)

2-(5-chloro-4-cyclopentyl-2-methyl-phenyl)-4,4,5,5-tetramethyl-1,3,2-dioxaborolane (Intermediate B-83) was prepared using procedure analogous to Intermediate B-80 (Step 1 and Step 2) by using cyclopentanol. H NMR (400 MHz, CDCl₃) δ 7.71 (s, 1H), 7.08 (s, 1H), 3.45-3.34 (m, 1H), 2.48 (s, 3H), 2.11-2.00 (m, 2H), 1.86-1.76 (m, 2H), 1.73-1.65 (m, 2H), 1.59-1.49 (m, 2H), 1.32 (s, 12H).

### Intermediate B-84

### 2-[4-(1-bicyclo[2.2.2]octanyl)-5-chloro-2-methyl-phenyl]-4,4,5,5-tetramethyl-1,3,2-dioxaborolane

### Step 1: 2-[4-(1-bicyclo[2.2.2]octanyl)-5-chloro-2-methyl-phenyl]-4,4,5,5-tetramethyl-1,3,2-dioxaborolane (Intermediate B-84)

2-[4-(1-bicyclo[2.2.2]octanyl)-5-chloro-2-methyl-phenyl]-4,4,5,5-tetramethyl-1,3,2-dioxaborolane (Intermediate B-84) was prepared using procedure analogous to Intermediate B-80 (Step 1 and Step 2) by using bicyclo[2.2.2]octan-1-ol. ESI-MS m/z calc. 360.20, found 361.3 (M+1)⁺. ¹H NMR (400 MHz, CDCl₃) δ 7.69 (s, 1H), 7.08 (s, 1H), 2.47 (s, 3H), 2.06-1.99 (m, 6H), 1.71-1.64 (m, 7H), 1.32 (s, 12H).

### Intermediate B-85

### 2-(5-chloro-2-methyl-4-norbornan-2-yl-phenyl)-4,4,5,5-tetramethyl-1,3,2-dioxaborolane

### Step 1: 2-(5-chloro-2-methyl-4-norbornan-2-yl-phenyl)-4,4,5,5-tetramethyl-1,3,2-dioxaborolane (Intermediate B-85)

2-(5-chloro-2-methyl-4-norbornan-2-yl-phenyl)-4,4,5,5-tetramethyl-1,3,2-dioxaborolane (Intermediate B-85) was prepared using procedure analogous to Intermediate B-80 (Step 1 and Step 2) by using norbornan-1-ol and used as crude for the subsequent step.

### Intermediate B-86

### 2-(5-chloro-2-methyl-4-phenyl-phenyl)-4,4,5,5-tetramethyl-1,3,2-dioxaborolane

### Step 1: 5-chloro-2-methyl-4-phenyl-phenol

A suspension of 4-bromo-5-chloro-2-methyl-phenol (150 mg, 0.67 mmol), phenylboronic acid (83 mg, 0.68 mmol) and sodium carbonate (215 mg, 2.03 mmol) in 1,4-dioxane (3 mL)/water (1.5 mL) in a microwave vial was degassed with nitrogen for 5 min. Pd(dppf)Cl₂·DCM (57 mg, 0.07 mmol) was added and the mixture was flushed with nitrogen and capped. The mixture was subjected to microwave irradiation at 130° C. for 30 min. The reaction mixture was diluted with EtOAc, washed with water and brine. The organic layer was dried over sodium sulfate, filtered and concentrated under reduced pressure. The crude material was purified by reverse phase chromatography (C₁₈) eluting with 10 to 99% MeCN in water (5 mM HCl) to give 5-chloro-2-methyl-4-phenyl-phenol (106 mg, 72%). ESI-MS m/z calc. 218.05, found 219.2 (M+1)⁺. ¹H NMR (400 MHz, DMSO-d₆) δ 9.88 (s, 1H), 7.48-7.25 (m, 5H), 7.11 (s, 1H), 6.92 (s, 1H), 2.13 (s, 3H).

### Step 2: 2-(5-chloro-2-methyl-4-phenyl-phenyl)-4,4,5,5-tetramethyl-1,3,2-dioxaborolane (Intermediate B-86)

2-(5-chloro-2-methyl-4-phenyl-phenyl)-4,4,5,5-tetramethyl-1,3,2-dioxaborolane (Intermediate B-86) was prepared form 5-chloro-2-methyl-4-phenyl-phenol using procedure analogous to Intermediate B-26 (Step 4 and Step 5). ESI-MS m/z calc. 328.14, found 329.3 (M+1)⁺. ¹H NMR (400 MHz, DMSO-d₆) δ 7.68 (s, 1H), 7.51-7.37 (m, 5H), 7.25 (s, 1H), 2.48 (s, 3H), 1.32 (s, 12H).

### Intermediate B-87

### 2-[3-chloro-2-fluoro-6-methyl-4-(2,2,2-trifluoro-1,1-dimethyl-ethyl)phenyl]-4,4,5,5-tetramethyl-1,3,2-dioxaborolane

### Step 1: 2-(2-chloro-3-fluoro-4-methoxy-phenyl)-1,1,1-trifluoro-propan-2-ol

To a solution at −78° C. of 1-bromo-2-chloro-3-fluoro-4-methoxy-benzene (3.15 g, 13.15 mmol) in ether (30 mL) was added a solution of n-BuLi in hexanes (5.8 mL of 2.5 M, 14.50 mmol). The reaction was stirred at −78° C. for 90 minutes, then 1,1,1-trifluoropropan-2-one (3.5 mL, 39.11 mmol) was added dropwise, and the reaction mixture was gradually warmed to room temperature and stirred overnight. The reaction mixture was quenched by slow addition of saturated aqueous ammonium chloride solution (30 mL). The layers were separated, and the aqueous layer was extracted with DCM (3×). The combined organic layers were dried over anhydrous magnesium sulfate, filtered, and concentrated under reduced pressure. Purification by silica gel column chromatography using 0 to 25% EtOAc in hexanes gave 2-(2-chloro-3-fluoro-4-methoxy-phenyl)-1,1,1-trifluoro-propan-2-ol (3 g, 84%). ESI-MS m/z calc. 272.02, found 255.1 (M−17)⁺. ¹H NMR (400 MHz, CDCl₃) δ 7.37 (dd, J=9.2, 2.2 Hz, 1H), 6.90 (t, J=8.6 Hz, 1H), 3.92 (s, 3H), 3.62 (s, 1H), 1.93 (s, 3H). ¹⁹F NMR (376 MHz, CDCl₃) δ −79.69, −131.32 (dd, J=8.2, 2.5 Hz).

### Step 2: 3-chloro-2-fluoro-1-methoxy-4-(2,2,2-trifluoro-1,1-dimethyl-ethyl)benzene

3-chloro-2-fluoro-1-methoxy-4-(2,2,2-trifluoro-1,1-dimethyl-ethyl)benzene was prepared from 2-(2-chloro-3-fluoro-4-methoxy-phenyl)-1,1,1-trifluoro-propan-2-ol using procedure analogousto that found in Intermediate B-3 (Step 3 and Step 4). ¹H NMR (400 MHz, CDCl₃) δ 7.26-7.20 (m, 1H), 6.84 (t, J=8.7 Hz, 1H), 3.90 (s, 3H), 1.75 (s, 6H). ¹⁹F NMR (376 MHz, CDCl₃) δ −74.29, −129.84 (dd, J=8.4, 2.5 Hz).

### Step 3: 3-chloro-2-fluoro-4-(2,2,2-trifluoro-1,1-dimethyl-ethyl)phenol

To a solution of 3-chloro-2-fluoro-1-methoxy-4-(2,2,2-trifluoro-1,1-dimethyl-ethyl)benzene (580 mg, 2.14 mmol) in DCM (6 mL) at −70° C. was added tribromoborane in DCM (3.5 mL of 1 M, 3.5 mmol). After 5 minutes the dry ice bath was removed and the reaction mixture was stirred at room temperature for 16 h. The reaction mixture was slowly quenched with methanol. The solvent was removed under reduced pressure and purification via silica gel column chromatography using 0 to 10% EtOAc in hexanes gave 3-chloro-2-fluoro-4-(2,2,2-trifluoro-1,1-dimethyl-ethyl)phenol (501 mg, 91%). ¹H NMR (400 MHz, CDCl₃) δ 7.22 (dq, J=9.1, 1.1 Hz, 1H), 6.90 (t, J=8.9 Hz, 1H), 5.24 (d, J=4.4 Hz, 1H), 1.74 (d, J=1.0 Hz, 6H). ¹⁹F NMR (376 MHz, CDCl₃) δ −74.41, −135.78-−135.87 (m).

### Step 4: 6-bromo-3-chloro-2-fluoro-4-(2,2,2-trifluoro-1,1-dimethyl-ethyl)phenol

To a suspension of 3-chloro-2-fluoro-4-(2,2,2-trifluoro-1,1-dimethyl-ethyl)phenol (390 mg, 1.52 mmol) in ACN (4 mL) was added 4-methylbenzenesulfonic acid monohydrate (30 mg, 0.16 mmol) and the reaction mixture was cooled to 0° C. NBS (320 mg, 1. 8 mmol) was added and the reaction mixture was gradually warmed to room temperature and stirred for 18 hours. The reaction mixture was quenched with a saturated aqueous solution of sodium bisulfite. The aqueous layer was extracted with EtOAc (2×), dried over magnesium sulfate, filtered and concentrated. Purification by silica gel column chromatography using hexanes gave 6-bromo-3-chloro-2-fluoro-4-(2,2,2-trifluoro-1,1-dimethyl-ethyl)phenol (112 mg, 22%) ¹H NMR (400 MHz, CDCl₃) δ 7.48-7.42 (m, 1H), 5.56 (d, J=2.6 Hz, 1H), 1.74 (s, 6H). ¹⁹F NMR (376 MHz, CDCl₃) δ −74.30, −127.73 (d, J=2.5 Hz).

### Step 5: 1-bromo-4-chloro-3-fluoro-2-methoxy-5-(2,2,2-trifluoro-1,1-dimethyl-ethyl)benzene

To a solution of 6-bromo-3-chloro-2-fluoro-4-(2,2,2-trifluoro-1,1-dimethyl-ethyl)phenol (138 mg, 0.41 mmol) in DMF (1.2 mL) was added potassium carbonate (115 mg, 0.83 mmol) and iodomethane (30 μL, 0.48 mmol) and the reaction mixture was stirred at room temperature for 18 hours. The reaction mixture was quenched with water and the aqueous layer was extracted with DCM (3×). The combined organic layer was dried over magnesium sulfate, filtered and concentrated. Purification via silica gel column chromatography using hexanes gave 1-bromo-4-chloro-3-fluoro-2-methoxy-5-(2,2,2-trifluoro-1,1-dimethyl-ethyl)benzene (130 mg, 90%). ¹H NMR (400 MHz, CDCl₃) δ 7.53-7.45 (m, 1H), 3.99 (d, J=1.7 Hz, 3H), 1.75 (s, 6H). ¹⁹F NMR (376 MHz, CDCl₃) δ −74.13, −121.70 (d, J=3.0 Hz).

### Step 6: 4-chloro-3-fluoro-2-methoxy-1-methyl-5-(2,2,2-trifluoro-1,1-dimethyl-ethyl)benzene

A microwave vial charged with 1-bromo-4-chloro-3-fluoro-2-methoxy-5-(2,2,2-trifluoro-1,1-dimethyl-ethyl)benzene (130 mg, 0.37 mmol), 2,4,6-trimethyl-1,3,5,2,4,6-trioxatriborinane (140 μL of 50% w/v, 0.56 mmol), Pd(dppf)Cl₂·DCM (32 mg, 0.04 mmol), tripotassium phosphate (235 mg, 1.11 mmol), water (120 μL) and dioxane (1.2 mL) was degassed under nitrogen, sealed and heated at 100° C. for 18 hours. The reaction mixture was filtered and concentrated. Purification via silica gel column chromatography using 0 to 5% EtOAc in hexanes gave 4-chloro-3-fluoro-2-methoxy-1-methyl-5-(2,2,2-trifluoro-1,1-dimethyl-ethyl)benzene (70 mg, 66%). ¹H NMR (400 MHz, CDCl₃) δ 7.09 (s, 1H), 3.92 (d, J=2.0 Hz, 3H), 2.25 (s, 3H), 1.74 (s, 6H). ¹⁹F NMR (376 MHz, CDCl₃) δ −74.09, −126.59.

### Step 7: 3-chloro-2-fluoro-6-methyl-4-(2,2,2-trifluoro-1,1-dimethyl-ethyl)phenol

To a solution of 4-chloro-3-fluoro-2-methoxy-1-methyl-5-(2,2,2-trifluoro-1,1-dimethyl-ethyl)benzene (70 mg, 0.25 mmol) in DCM (700 μL) at −70° C. was added tribromoborane in DCM (400 μL of 1 M, 0.4000 mmol). After 5 min, the reaction mixture was stirred at room temperature for 16 h, poured onto ice and extracted with DCM. The organic layer was separated, dried over magnesium sulfate, filtered and concentrated. The solvent was removed under reduced pressure. The crude material was purified via silica gel column chromatography using 0 to 10% EtOAc in hexanes to obtain 3-chloro-2-fluoro-6-methyl-4-(2,2,2-trifluoro-1,1-dimethyl-ethyl)phenol (52 mg, 78%). ¹H NMR (400 MHz, CDCl₃) δ 7.07 (s, 1H), 5.18 (d, J=5.1 Hz, 1H), 2.26 (s, 3H), 1.73 (s, 6H). ¹⁹F NMR (376 MHz, CDCl₃) δ −74.33, −136.75 (dd, J=5.2, 2.1 Hz).

### Step 8: 2-[3-chloro-2-fluoro-6-methyl-4-(2,2,2-trifluoro-1,1-dimethyl-ethyl)phenyl]-4,4,5,5-tetramethyl-1,3,2-dioxaborolane (Intermediate B-87)

2-[3-chloro-2-fluoro-6-methyl-4-(2,2,2-trifluoro-1,1-dimethyl-ethyl)phenyl]-4,4,5,5-tetramethyl-1,3,2-dioxaborolane (Intermediate B-87) was prepared from 3-chloro-2-fluoro-6-methyl-4-(2,2,2-trifluoro-1,1-dimethyl-ethyl)phenol using procedure analogous to that found in Intermediate B-26 (Step 4 and Step 5). ESI-MS m/z calc. 380.13, found 381.2 (M+1)⁺.

### Intermediate B-88

### 2-[5-chloro-2-methyl-4-(trifluoromethoxy)phenyl]-4,4,5,5-tetramethyl-1,3,2-dioxaborolane

### Step 1: 5-chloro-2-iodo-4-(trifluoromethoxy)phenol

To 3-chloro-4-(trifluoromethoxy)phenol (8.4 g, 29.64 mmol) in acetic acid (11.61 g, 11 mL, 193.43 mmol) was added N-Iodosuccinimide (6.7 g, 29.78 mmol). The mixture was stirred at room temperature for 5 minutes and then sulfuric acid (1.17 g, 0.65 mL, 11.71 mmol) was added and the reaction was stirred at room temperature for 16 h. The reaction was diluted with diethyl ether (50 mL), washed with water (2×50 mL) then 10% aqueous sodium thiosulfate solution (100 mL) and concentrated under reduced pressure. Purification by silica gel column chromatography using 0 to 20% EtOAc in heptane) yielded 5-chloro-2-iodo-4-(trifluoromethoxy)phenol (10.2 g, 88%). ESI-MS m/z calc. 337.88, found 336.86 (M−1)⁻. ¹H-NMR (400 MHz, CDCl₃) δ 7.62 (q, J=1.1 Hz, 1H), 7.13 (s, 1H), 5.38 (s, 1H).

### Step 2: 1-chloro-4-iodo-5-[(4-methoxyphenyl)methoxy]-2-(trifluoromethoxy)benzene

To a solution of 5-chloro-2-iodo-4-(trifluoromethoxy)phenol (10.2 g, 25.96 mmol) in acetone (230 mL) with potassium carbonate (5 g, 36.18 mmol), tetrabutylammonium iodide (670 mg, 1.81 mmol) and 18-crown-6 (400 mg, 1.51 mmol) was added 1-(chloromethyl)-4-methoxy-benzene (4.95 g, 31.6 mmol). The reaction mixture was refluxed for 16 h. The reaction was cooled to room temperature then concentrated under reduced pressure before being partitioned between water (100 mL) and ethyl acetate (100 mL). The aqueous layer was extracted with EtOAc (100 mL). The combined organic layer was washed with brine (100 mL), dried over sodium sulfate, filtered and concentrated under reduced pressure. Purification by silica gel column chromatography using 0 to 5% EtOAc in heptane yielded 1-chloro-4-iodo-5-[(4-methoxyphenyl)methoxy]-2-(trifluoromethoxy)benzene (9.8 g, 70%). ¹H-NMR (400 MHz, CDCl₃) δ 7.75 (d, J=1.1 Hz, 1H), 7.43-7.40 (m, 2H), 6.98-6.95 (m, 3H), 5.08 (s, 2H), 3.86 (s, 3H)

### Step 3: 1-chloro-5-[(4-methoxyphenyl)methoxy]-4-methyl-2-(trifluoromethoxy)benzene

To a solution of 1-chloro-4-iodo-5-[(4-methoxyphenyl)methoxy]-2-(trifluoromethoxy)benzene (145 mg, 0.31 mmol) in 1,4-dioxane (2.4 mL) and water (800 μL) was added methylboronic acid (23 mg, 0.38 mmol) and potassium phosphate tribasic (135 mg, 0.63 mmol). The mixture was degassed with argon for 5 minutes then Pd(dppf)Cl₂ (12 mg, 0.01 mmol) was added. The reaction mixture was subjected to microwave irradiation for 1 hour at 100° C. The reaction mixture was cooled to room temperature, diluted with EtOAc (15 mL), washed with aqueous saturated sodium bicarbonate (15 mL), water (15 mL), brine (10 mL), dried over sodium sulfate, filtered and concentrated under reduced pressure to give 1-chloro-5-[(4-methoxyphenyl)methoxy]-4-methyl-2-(trifluoromethoxy)benzene (95 mg, 79%). ¹H-NMR (400 MHz, CDCl₃) δ 7.39-7.36 (m, 1H), 7.29 (s, 1H), 7.12 (s, 1H), 6.98-6.94 (m, 3H), 5.00 (s, 2H), 3.86 (s, 3H), 2.24 (s, 3H).

### Step 4: 5-chloro-2-methyl-4-(trifluoromethoxy)phenol

To 1-chloro-5-[(4-methoxyphenyl)methoxy]-4-methyl-2-(trifluoromethoxy)benzene (50 mg, 0.13 mmol) in DCM (3 mL) was added TFA (14.8 mg, 0.01 mL, 0.13 mmol). The reaction mixture was stirred at room temperature for 40 h. The reaction mixture was concentrated under reduced pressure and azeotroped with toluene (3×10 mL) to give 5-chloro-2-methyl-4-(trifluoromethoxy)phenol (30 mg, 92%). ¹H-NMR (400 MHz, CDCl₃) δ 7.10 (s, 1H), 6.90 (s, 1H), 3.25 (br s, 1H), 2.25 (s, 3H).

### Step 5: 2-[5-chloro-2-methyl-4-(trifluoromethoxy)phenyl]-4,4,5,5-tetramethyl-1,3,2-dioxaborolane (Intermediate B-88)

2-[5-chloro-2-methyl-4-(trifluoromethoxy)phenyl]-4,4,5,5-tetramethyl-1,3,2-dioxaborolane (Intermediate B-88, 339 mg, 62%) was prepared from 5-chloro-2-methyl-4-(trifluoromethoxy)phenol using procedure analogous to Intermediate B-26 (Step 4 and Step 5). ¹H-NMR (400 MHz, CDCl₃) δ 7.84 (s, 1H), 7.11 (s, 1H), 2.53 (s, 3H), 1.35 (s, 12H).

### Intermediate B-89

### 2-[5-chloro-2-methyl-4-[1-(trifluoromethyl)cyclopentyl]phenyl]-4,4,5,5-tetramethyl-1,3,2-dioxaborolane

### Step 1: 1-(2-chloro-4-methoxy-5-methyl-phenyl)-2,2,2-trifluoro-ethanol

To a stirred mixture of 1-bromo-2-chloro-4-methoxy-5-methyl-benzene (5 g, 21.2 mmol) in diethyl ether (110 mL) at −78° C. was added n-BuLi solution (in hexanes) (9.5 mL of 2.5 M, 23.75 mmol). The reaction mixture was stirred at this temperature for 1 h then methyl trifluoroacetate (3.31 g, 2.6 mL, 25.85 mmol) was added dropwise. The reaction mixture was stirred at this temperature for 1 h warmed up to room temperature and stirred for 16 h. The reaction mixture was quenched with saturated aqueous ammonium chloride solution (90 mL) then poured in a separatory funnel with water (45 mL) and diethyl ether (45 mL). The layers were separated and the aqueous layer was extracted with diethyl ether (50 mL). The combined organic layers were dried over anhydrous sodium sulfate, filtered and concentrated under reduced pressure. The residue was purified by silica gel flash chromatography using 0 to 30% ethyl acetate in heptane to give 1-(2-chloro-4-methoxy-5-methyl-phenyl)-2,2,2-trifluoro-ethanol (2.86 g, 52%). H NMR (400 MHz, CDCl₃) δ 7.40 (s, 1H), 6.83 (s, 1H), 5.53 (dq, J=6.4, 5.1 Hz, 1H), 3.83 (s, 3H), 2.56 (d, J=4.9 Hz, 1H), 2.20 (s, 3H). 19 F NMR (377 MHz, CDCl₃) δ −77.96 (d, J=5.4 Hz, 3F).

### Step 2: 1-(2-chloro-4-methoxy-5-methyl-phenyl)-2,2,2-trifluoro-ethanone

To a solution of 1-(2-chloro-4-methoxy-5-methyl-phenyl)-2,2,2-trifluoro-ethanol (1 g, 3.82 mmol) in DCM (20 mL) was added Dess-Martin periodinane (5 g, 10.61 mmol) and sodium carbonate (1.3 g, 12.26 mmol). The solution was stirred at room temperature for 16 h. Water (50 mL) was added, the mixture was stirred for 30 min then it was then extracted with DCM (2×60 mL). The combined organic layer was dried over anhydrous sodium sulfate, filtered and concentrated under reduced pressure. The residue was purified by silica gel flash chromatography using 0 to 20% ethyl acetate in heptane to give 1-(2-chloro-4-methoxy-5-methyl-phenyl)-2,2,2-trifluoro-ethanone (710 mg, 70%). ESI-MS m/z calc. 252.01, found 253.1 (M+1)⁺. ¹H NMR (400 MHz, CDCl₃) δ 7.60 (s, 1H), 6.96 (s, 1H), 3.92 (s, 3H), 2.23 (s, 3H). ¹⁹F NMR (377 MHz, CDCl₃) δ −71.31 (s, 3F).

### Step 3: 2-(2-chloro-4-methoxy-5-methyl-phenyl)-1,1,1-trifluoro-pent-4-en-2-ol

To a solution of 1-(2-chloro-4-methoxy-5-methyl-phenyl)-2,2,2-trifluoro-ethanone (650 mg, 2.57 mmol) in diethyl ether (13 mL) at 0° C. was added allylmagnesium bromide in diethyl ether (4 mL of 1 M, 4 mmol) and the reaction was stirred at this temperature for 2 hours. The reaction was slowly quenched with saturated aqueous ammonium chloride (20 mL) and extracted with MTBE (2×50 mL). The combined organic layer was dried over anhydrous sodium sulfate, filtered and concentrated under reduced pressure to give 2-(2-chloro-4-methoxy-5-methyl-phenyl)-1,1,1-trifluoro-pent-4-en-2-ol (788 mg, 97%). ESI-MS m/z calc. 294.06, found 277.1 (M−17)⁺. ¹H NMR (400 MHz, CDCl₃) δ 7.47 (s, 1H), 6.81 (s, 1H), 5.72-5.60 (m, 1H), 5.27 (dq, J=17.1, 1.5 Hz, 1H), 5.22-5.17 (m, 1H), 3.83 (s, 3H), 3.58 (dd, J=14.8, 6.5 Hz, 1H), 3.38 (s, 1H), 2.82 (dd, J=14.8, 7.7 Hz, 1H), 2.18 (s, 3H). ¹⁹F NMR (377 MHz, CDCl₃) δ −78.87 (s, 3F).

### Step 4: 1-[1-allyl-1-(trifluoromethyl)but-3-enyl]-2-chloro-4-methoxy-5-methyl-benzene

To an unstirred solution of 2-(2-chloro-4-methoxy-5-methyl-phenyl)-1,1,1-trifluoro-pent-4-en-2-ol (540 mg, 1.72 mmol) in DCE (9 mL) at room temperature was added indium bromide (200 mg, 0.56 mmol). A solution of allyl(trimethyl)silane (1 g, 1.4 mL, 8.75 mmol) in DCE (4 mL) was added over 50 minutes using a syringe-pump. Once the addition was started, the reaction mixture was stirred. After the end of addition, the reaction mixture was stirred for an additional 1.5 h, quenched with saturated aqueous sodium bicarbonate (5 mL), water (30 mL) was added and the aqueous layer was extracted with DCM (2×50 mL). The combined organic layer was dried over anhydrous sodium sulfate, filtered and concentrated under reduced pressure. Purification by silica gel flash chromatography using 0 to 15% ethyl acetate in heptane gave 1-[1-allyl-1-(trifluoromethyl)but-3-enyl]-2-chloro-4-methoxy-5-methyl-benzene (294 mg, 53%). ESI-MS m/z calc. 318.1, found 319.1 (M+1)⁺. ¹H NMR (400 MHz, CDCl₃) δ 7.22 (s, 1H), 6.82 (s, 1H), 5.69-5.55 (m, 2H), 5.11 (dq, J=17.0, 1.7 Hz, 2H), 5.02-4.94 (m, 2H), 3.81 (s, 3H), 3.15 (dd, J=15.4, 7.1 Hz, 2H), 2.93 (dd, J=15.4, 7.3 Hz, 2H), 2.16 (s, 3H). ¹⁹F NMR (377 MHz, CDCl₃) δ −67.52 (s, 3F).

### Step 5: 1-chloro-5-methoxy-4-methyl-2-[1-(trifluoromethyl)cyclopent-3-en-1-yl]benzene

A solution of 1-[1-allyl-1-(trifluoromethyl)but-3-enyl]-2-chloro-4-methoxy-5-methyl-benzene (293 mg, 0.90 mmol) and Grubbs catalyst, 2nd generation (38 mg, 0.04 mmol) in nitrogen degassed DCE (90 mL) was stirred at 40° C. for 2.5 hours. Once cooled to room temperature, the crude reaction mixture was concentrated under reduced pressure and the residue was filtered over a pad of silica gel using DCM/heptane 9/1 mixture as an eluent. The filtrate was concentrated under reduced pressure and dried in-vacuo to give 1-chloro-5-methoxy-4-methyl-2-[1-(trifluoromethyl)cyclopent-3-en-1-yl]benzene (273 mg, 96%). ESI-MS m/z calc. 290.07, found 291.1 (M+1)⁺. ¹H NMR (400 MHz, CDCl₃) δ 7.15 (s, 1H), 6.82 (s, 1H), 5.74 (s, 2H), 3.81 (s, 3H), 3.31-3.21 (m, 2H), 3.14-3.04 (m, 2H), 2.18 (s, 3H). ¹⁹F NMR (377 MHz, CDCl₃) δ −76.55 (s, 3F). GC-MS m/z calc. 290.06, found 289.90 (M)+.

### Step 6: 1-chloro-5-methoxy-4-methyl-2-[1-(trifluoromethyl)cyclopentyl]benzene

To a solution of 1-chloro-5-methoxy-4-methyl-2-[1-(trifluoromethyl)cyclopent-3-en-1-yl]benzene (250 mg, 0.79 mmol) in ethyl acetate (8 mL) under nitrogen atmosphere was added palladium on carbon (160 mg, 0.07 mmol). The mixture was sparged with hydrogen for 5 min then it was stirred under hydrogen atmosphere for 1.75 h. The reaction mixture was filtered over a short pad of Celite and the filtrate was concentrated under reduced pressure then dried in-vacuo to give 1-chloro-5-methoxy-4-methyl-2-[1-(trifluoromethyl)cyclopentyl]benzene (241 mg, 87%) as a colorless oil that crystallized to a solid. ESI-MS m/z calc. 292.08, found 293.1 (M+1)⁺. ¹H NMR (400 MHz, CDCl₃) δ 7.18 (s, 1H), 6.81 (s, 1H), 3.81 (s, 3H), 2.76-2.65 (m, 2H), 2.32-2.19 (m, 2H), 2.17 (s, 3H), 1.92-1.81 (m, 2H), 1.81-1.69 (m, 2H). ¹⁹F NMR (377 MHz, CDCl₃) δ −72.19 (s, 3F).

### Step 7: 5-chloro-2-methyl-4-[1-(trifluoromethyl)cyclopentyl]phenol

To a solution of 1-chloro-5-methoxy-4-methyl-2-[1-(trifluoromethyl)cyclopentyl]benzene (240 mg, 0.69 mmol) in DCM (3 mL) at 0° C. was added BBr₃ (211.20 mg, 80 μL, 0.84 mmol). The resulting mixture was stirred at this temperature for 3 h and then slowly warmed up to room temperature and stirred for 16 h. The reaction was quenched by addition of MeOH (10 mL) and was concentrated under reduced pressure. The residue was taken in MTBE (50 mL) and washed with saturated aqueous sodium bicarbonate solution (20 mL) then water (20 mL). The organic layer was dried over anhydrous sodium sulfate, filtered and concentrated under reduced pressure. Purification by silica gel flash chromatography using 0 to 20% ethyl acetate in heptane followed by a second purification by reverse phase flash chromatography (C₁₈) using 2 to 80% MeCN in water (0.1% formic acid) gave 5-chloro-2-methyl-4-[1-(trifluoromethyl)cyclopentyl]phenol (173 mg, 90%) as a colorless oil that crystallized to a white solid. ESI-MS m/z calc. 278.07, found 279.1 (M+1)⁺. ¹H NMR (400 MHz, CDCl₃) δ 7.18 (s, 1H), 6.82 (s, 1H), 4.86 (s, 1H), 2.75-2.65 (m, 2H), 2.29-2.17 (m, 5H), 1.92-1.81 (m, 2H), 1.81-1.69 (m, 2H). ¹⁹F NMR (377 MHz, CDCl₃) δ −72.20 (s, 3F).

### Step 8: 2-[5-chloro-2-methyl-4-[1-(trifluoromethyl)cyclopentyl]phenyl]-4,4,5,5-tetramethyl-1,3,2-dioxaborolane (Intermediate B-89)

2-[5-chloro-2-methyl-4-[1-(trifluoromethyl)cyclopentyl]phenyl]-4,4,5,5-tetramethyl-1,3,2-dioxaborolane (Intermediate B-89) was prepared from 5-chloro-2-methyl-4-[1-(trifluoromethyl)cyclopentyl]phenol using procedure analogous to Intermediate B-26 (Step 4 and Step 5). ESI-MS m/z calc. 388.16, found 389.2 (M+1)⁺.

### Intermediate B-90

### tert-butyl-dimethyl-[2-methyl-2-[5-methyl-4-(4,4,5,5-tetramethyl-1,3,2-dioxaborolan-2-yl)-2-(trifluoromethyl)phenyl]propoxy]silane

### Step 1: 2-[4-benzyloxy-5-methyl-2-(trifluoromethyl)phenyl]propan-2-ol

To a solution of 1-benzyloxy-4-bromo-2-methyl-5-(trifluoromethyl)benzene (500 mg, 1.39 mmol) in diethyl ether (6 mL) at −78° C. was added a solution of n-BuLi in hexanes (0.6 mL of 2.5 M, 1.5 mmol). The reaction was stirred at −78° C. for 1 h, then acetone (277 mg, 350 μL, 4.77 mmol) was added dropwise, and it was stirred for 2 h at −78° C. The dry-ice bath was removed and the reaction mixture was quenched by slow addition of ammonium chloride saturated aqueous solution (30 mL). MTBE (30 mL) was added to the mixture and the layers were separated and the aqueous layer was extracted with MTBE (2×30 mL). The combined organic layers were dried over anhydrous sodium sulfate, filtered and concentrated under reduced pressure. Purification by silica gel flash chromatography using 0 to 10% EtOAc in heptanes afforded 2-[4-benzyloxy-5-methyl-2-(trifluoromethyl)phenyl]propan-2-ol (240 mg, 50%) as a tan solid. ESI-MS m/z calc. 324.13, found 307.2 (M−17)⁺. ¹H NMR (400 MHz, CDCl₃) δ 7.48-7.32 (m, 6H), 7.24 (s, 1H), 5.11 (s, 2H), 2.31 (s, 3H), 1.96 (s, 1H), 1.67 (s, 6H). ¹⁹F NMR (377 MHz, CDCl₃) δ −53.84 (s, 3F).

### Step 2: 2-[4-benzyloxy-5-methyl-2-(trifluoromethyl)phenyl]-2-methyl-propanenitrile

To a solution of trimethylsilylformonitrile (94.8 mg, 0.12 mL, 0.96 mmol) and indium bromide (15 mg, 0.04 mmol) in DCM (0.5 mL) at room temperature was added a solution of 2-[4-benzyloxy-5-methyl-2-(trifluoromethyl)phenyl]propan-2-ol (100 mg, 0.3 mmol) in DCM (1 mL) over 5 minutes and the solution was stirred at room temperature for 15 minutes. The reaction mixture was concentrated under reduced pressure and co-evaporated with DCM (2×30 mL). The crude product was purified by silica gel chromatography using 0 to 10% EtOAc in heptanes to afford 2-[4-benzyloxy-5-methyl-2-(trifluoromethyl)phenyl]-2-methyl-propanenitrile (75 mg, 73%) as a colorless oil. ESI-MS m/z calc. 333.13, found 334.2 (M+1)⁺.

### Step 3: 2-[4-benzyloxy-5-methyl-2-(trifluoromethyl)phenyl]-2-methyl-propanal

To a solution of 2-[4-benzyloxy-5-methyl-2-(trifluoromethyl)phenyl]-2-methyl-propanenitrile (2.39 g, 4.72 mmol) in THF (60 mL) cooled at 0° C. was slowly added diisobutylaluminum hydride solution in hexanes (11.3 mL of 1 M, 11.3 mmol). The reaction was slowly warmed to room temperature and stirred for 4 hours. The reaction mixture was quenched with an aqueous solution of HCl 1M (50 mL) and diluted with DCM (50 mL). The organic layer was washed with water, dried over anhydrous sodium sulfate, filtered and concentrated under reduced pressure. It was purified by silica gel flash chromatography using 100% heptanes to afford 2-[4-benzyloxy-5-methyl-2-(trifluoromethyl)phenyl]-2-methyl-propanal (1.5 g, 71%) as a colorless oil. ESI-MS m/z calc. 336.13, found 337.1 (M+1)⁺. ¹H NMR (400 MHz, DMSO-d₆) δ 9.56 (q, J=2.6 Hz, 1H), 7.56-7.32 (m, 6H), 7.29 (s, 1H), 5.22 (s, 2H), 2.30 (s, 3H), 1.41 (s, 6H). ¹⁹F NMR (377 MHz, DMSO-d₆) δ −53.29 (s, 3F).

### Step 4: 2-[4-benzyloxy-5-methyl-2-(trifluoromethyl)phenyl]-2-methyl-propan-1-ol

To a solution of 2-[4-benzyloxy-5-methyl-2-(trifluoromethyl)phenyl]-2-methyl-propanal (210 mg, 0.44 mmol) in tetrahydrofuran (5 mL) at 0° C. was added sodium borohydride (70 mg, 1.85 mmol). The mixture was stirred at room temperature for 20 h. The reaction mixture was quenched with water (50 mL) and extracted with DCM (2×30 mL). The combined organic layer was washed with brine (50 mL), dried over anhydrous sodium sulfate, filtered and concentrated under reduced pressure. Purification by silica gel flash chromatography using 0 to 10% EtOAc in heptanes afforded 2-[4-benzyloxy-5-methyl-2-(trifluoromethyl)phenyl]-2-methyl-propan-1-ol (140 mg, 92%) as a colorless oil. ESI-MS m/z calc. 338.1494, found 321.2 (M−17)⁺. ¹H NMR (400 MHz, DMSO-d₆) δ 7.50-7.30 (m, 6H), 7.25 (s, 1H), 5.18 (s, 2H), 4.75 (t, J=5.4 Hz, 1H), 3.53 (d, J=5.6 Hz, 2H), 2.25 (s, 3H), 1.30 (s, 6H). ¹⁹F NMR (377 MHz, DMSO-d₆) δ −51.17 (s, 3F).

### Step 5: 4-(2-hydroxy-1,1-dimethyl-ethyl)-2-methyl-5-(trifluoromethyl)phenol

A solution of 2-[4-benzyloxy-5-methyl-2-(trifluoromethyl)phenyl]-2-methyl-propan-1-ol (140 mg, 0.40 mmol) in methanol (5 mL) was sparged with nitrogen for 10 minutes. Then, palladium on carbon (10% w/w) (68 mg, 0.06 mmol) was added and the reaction mixture was bubbled with hydrogen for 5 minutes. The reaction was stirred at room temperature for 1 hour under an atmosphere of hydrogen. The mixture was sparged with nitrogen for 5 minutes, filtered through Celite®, washed with MeOH and the filtrate concentrated under reduced pressure to afford 4-(2-hydroxy-1,1-dimethyl-ethyl)-2-methyl-5-(trifluoromethyl)phenol (100 mg, 94%). ESI-MS m/z calc. 248.10, found 231.2 (M−17)⁺. ¹H NMR (400 MHz, DMSO-d₆) δ 9.99 (br. s., 1H), 7.34 (s, 1H), 7.13 (s, 1H), 4.70 (br. s., 1H), 3.49 (s, 2H), 2.15 (s, 3H), 1.28 (s, 6H). ¹⁹F NMR (377 MHz, DMSO-d₆) δ −51.34 (s, 3F).

### Step 6: 4-[2-[tert-butyl(dimethyl)silyl]oxy-1,1-dimethyl-ethyl]-2-methyl-5-(trifluoromethyl)phenol

To a mixture of 4-(2-hydroxy-1,1-dimethyl-ethyl)-2-methyl-5-(trifluoromethyl)phenol (60 mg, 0.24 mmol) and dichloromethane (2 mL) were added imidazole (20 mg, 0.3 mmol) and tert-butyldimethylsilyl chloride (45 mg, 0.3 mmol). The reaction mixture was stirred for 20 h at room temperature, then another portion of tert-butyldimethylsilyl chloride (12 mg, 0.08 mmol) was added and the reaction mixture was stirred at room temperature for additional 24 hours. The reaction mixture was filtered over celite and rinsed with heptanes (20 mL). The filtrate was concentrated under reduced pressure and the crude material was purified by silica gel chromatography using 0 to 20% EtOAc in heptanes to afford 4-[2-[tert-butyl(dimethyl)silyl]oxy-1,1-dimethyl-ethyl]-2-methyl-5-(trifluoromethyl)phenol (25 mg, 29%) as a white solid. ESI-MS m/z calc. 362.19, found 231.2 (M−131)⁺. H NMR (400 MHz, DMSO-d₆) δ 9.70 (s, 1H), 7.34 (s, 1H), 7.13 (s, 1H), 3.62 (s, 2H), 2.14 (s, 3H), 1.31 (s, 6H), 0.79 (s, 9H), −0.08 (s, 6H). ¹⁹F NMR (377 MHz, DMSO-d₆) δ −51.42 (s, 3F).

### Step 7: tert-butyl-dimethyl-[2-methyl-2-[5-methyl-4-(4,4,5,5-tetramethyl-1,3,2-dioxaborolan-2-yl)-2-(trifluoromethyl)phenyl]propoxy]silane (Intermediate B-90)

tert-butyl-dimethyl-[2-methyl-2-[5-methyl-4-(4,4,5,5-tetramethyl-1,3,2-dioxaborolan-2-yl)-2-(trifluoromethyl)phenyl]propoxy]silane (Intermediate B-90, 193 mg, 100%) was prepared from 4-[2-[tert-butyl(dimethyl)silyl]oxy-1,1-dimethyl-ethyl]-2-methyl-5-(trifluoromethyl)phenol using procedure analogous to that found in Intermediate B-26 (Step 4 and Step 5). ESI-MS m/z calc. 472.2792, found 341.2 (M−131)⁺. ¹H NMR (400 MHz, CDCl₃) δ 8.09 (s, 1H), 7.44 (s, 1H), 3.70-3.68 (m, 2H), 2.55 (s, 3H), 1.42-1.39 (m, 6H), 1.28 (s, 12H), 0.84 (s, 9H), −0.05 (s, 6H). ¹⁹F NMR (377 MHz, CDCl₃) δ −52.17 (s, 3F).

### Intermediate B-91

### Methyl 5-tert-butyl-4-chloro-2-(4,4,5,5-tetramethyl-1,3,2-dioxaborolan-2-yl)benzoate

### Step 1: 4-tert-butyl-3-chloro-phenol

A solution of 4-tert-butyl-3-chloro-aniline (5 g, 27.2 mmol) in sulfuric acid (40 mL of 12 M, 480 mmol), cycloheptylmethyl ether (80 mL) and water (40 mL) was stirred at 50° C. until the complete dissolution then cooled to room temperature. A solution of sodium nitrite (2.0 g, 29 mmol) in water (30 mL) was added very slowly at 10° C. and reaction mixture was stirred for 1 hour at room temperature. The reaction mixture was stirred at 90° C. for 75 minutes. Once cooled to room temperature, the reaction mixture was diluted with water (100 mL) and extracted with ethyl acetate (3×150 mL). The combined organic layer was dried over sodium sulfate, filtered and concentrated under reduced pressure. The residue was purified by silica gel column chromatography using 0 to 20% of EtOAc in heptanes to afford 4-tert-butyl-3-chloro-phenol (4.93 g, 98%). ESI-MS m/z calc. 184.06, found 183.0 (M−1)⁻. ¹H NMR (400 MHz, CDCl₃) δ 7.28 (d, J=8.6 Hz, 1H), 6.88 (d, J=2.7 Hz, 1H), 6.68 (dd, J=8.7, 2.8 Hz, 1H), 4.85 (br. s, 1H), 1.45 (s, 9H).

### Step 2: 4-tert-butyl-5-chloro-2-iodo-phenol

p-Toluenesulfonic acid (monohydrate) (5.1 g, 26.81 mmol) was added to a solution of 4-tert-butyl-3-chloro-phenol (4.93 g, 26.56 mmol) in acetonitrile (50 mL) at room temperature. After stirring 10 minutes, Nis (6 g, 26.67 mmol) was added and reaction mixture was stirred at room temperature overnight. The reaction mixture was quench with aqueous solution of sodium thiosulfate (100 mL), diluted with aqueous 1M HCl (10 mL) and extracted with ethyl acetate (3×100 mL). Organic layers were combined, washed with brine (50 mL), dried over sodium sulfate and concentrated under reduced pressure. The residue was purified by silica gel column chromatography using 0 to 10% of EtOAc in heptanes to afford 4-tert-butyl-5-chloro-2-iodo-phenol (5.34 g, 65%) as a clear oil. ESI-MS m/z calc. 309.96, found 308.9 (M−1)⁻. ¹H NMR (400 MHz, CDCl₃) δ 7.65 (s, 1H), 7.03 (s, 1H), 5.17 (s, 1H), 1.45 (s, 9H).

### Step 3: methyl 5-tert-butyl-4-chloro-2-hydroxy-benzoate

In a reactor at room temperature, a solution of 4-tert-butyl-5-chloro-2-iodo-phenol (5.34 g, 17.2 mmol), bis(triphenylphosphine)palladium(II)chloride (660 mg, 0.94 mmol), methanol (126.56 g, 160 mL, 4 mol) and triethylamine (5.44 g, 7.5 mL, 53.81 mmol) in dimethylformamide (160 mL) was placed under 30 PSI of carbon monoxide (5 g, 178.51 mmol) and heated at 60° C. overnight. Once cooled to room temperature, the reaction mixture was diluted with water (1 L) and extracted using ethyl acetate (3×300 mL). The combined organic layer was washed with water (3×150 mL) and brine (200 mL), dried over sodium sulfate and concentrated under reduced pressure. The residue was purified by silica gel chromatography using 0 to 10% of EtOAc in heptanes to afford methyl 5-tert-butyl-4-chloro-2-hydroxy-benzoate (3.71 g, 89%) as a white solid. ESI-MS m/z calc. 242.07, found 243.2 (M+1)⁺. ¹H NMR (400 MHz, CDCl₃) δ 10.57 (s, 1H), 7.89 (s, 1H), 7.05 (s, 1H), 3.98 (s, 3H), 1.48 (s, 9H).

### Step 4: methyl 5-tert-butyl-4-chloro-2-(4,4,5,5-tetramethyl-1,3,2-dioxaborolan-2-yl)benzoate (Intermediate B-91)

Methyl 5-tert-butyl-4-chloro-2-(4,4,5,5-tetramethyl-1,3,2-dioxaborolan-2-yl)benzoate (Intermediate B-91, 307 mg, 98%) was prepared from 5-tert-butyl-4-chloro-2-hydroxy-benzoate using procedure analogous to Intermediate B-26 (Step 4 and Step 5) as a yellow oil. ESI-MS m/z calc. 352.1613, found 353.2 (M+1)⁺. ¹H NMR (400 MHz, CDCl₃) δ 8.02 (s, 1H), 7.47 (s, 1H), 3.93 (s, 3H), 1.50 (s, 9H), 1.43 (s, 12H).

### Intermediate B-92

### [5-tert-butyl-4-chloro-2-(4,4,5,5-tetramethyl-1,3,2-dioxaborolan-2-yl)phenyl]methanol

### Step 1: [5-tert-butyl-4-chloro-2-(4,4,5,5-tetramethyl-1,3,2-dioxaborolan-2-yl)phenyl]methanol (Intermediate B-92)

A solution of DIBAL (in hexanes) (2.4 mL of 1 M, 2.4 mmol) was slowly added to a solution of methyl 5-tert-butyl-4-chloro-2-(4,4,5,5-tetramethyl-1,3,2-dioxaborolan-2-yl)benzoate (307 mg, 0.82 mmol) in tetrahydrofuran (5 mL) at −78° C. and reaction mixture was stirred at −78° C. for 1 hour. The reaction mixture was quenched by addition of methanol (5 mL) and concentrated under reduced pressure to afford [5-tert-butyl-4-chloro-2-(4,4,5,5-tetramethyl-1,3,2-dioxaborolan-2-yl)phenyl]methanol (Intermediate B-92).

### Intermediate B-93

### tert-butyl-[2-[2-chloro-5-methyl-4-(4,4,5,5-tetramethyl-1,3,2-dioxaborolan-2-yl)phenyl]-3,3,3-trifluoro-2-methyl-propoxy]-dimethyl-silane

### Step 1: 2-(2-chloro-4-methoxy-5-methyl-phenyl)-1,1,1-trifluoro-propan-2-ol

To a stirred mixture at −78° C. of 1-bromo-2-chloro-4-methoxy-5-methyl-benzene (10.5 g, 44.54 mmol) in diethyl ether (180 mL) was added a solution of n-BuLi in hexanes (20 mL of 2.5 M, 50 mmol) slowly over 15 minutes. The reaction mixture was stirred at this temperature for 45 minutes then a solution of 1,1,1-trifluoropropan-2-one (10 g, 8 mL, 89.4 mmol) in diethyl ether (10 mL) was added slowly over 15 minutes. The reaction mixture was stirred at −78° C. for 1 h, then the dry-ice bath was removed and it was stirred at room temperature for 3 h. The reaction was quenched by slow addition of saturated aqueous ammonium chloride solution (200 mL) and water (100 mL). The layers were separated and the organic layer was washed with brine (100 mL), dried over anhydrous sodium sulfate, filtered and concentrated under reduced pressure. The crude product was purified by silica gel flash chromatography using 0 to 25% EtOAc in heptanes to afford 2-(2-chloro-4-methoxy-5-methyl-phenyl)-1,1,1-trifluoro-propan-2-ol (10.77 g, 77%) as a yellow oil. ESI-MS m/z calc. 268.05, found 251.5 (M−17)⁺. ¹H NMR (400 MHz, CDCl₃) δ 7.32 (s, 1H), 6.82 (s, 1H), 4.05 (s, 1H), 3.84 (s, 3H), 2.20 (s, 3H), 1.89 (s, 3H). ¹⁹F NMR (377 MHz, CDCl₃) δ −79.90 (s, 3F).

### Step 2: 1-chloro-2-(1-chloro-2,2,2-trifluoro-1-methyl-ethyl)-5-methoxy-4-methyl-benzene

To a solution of 2-(2-chloro-4-methoxy-5-methyl-phenyl)-1,1,1-trifluoro-propan-2-ol (100 mg, 0.2434 mmol) and triethylamine (87 mg, 0.12 mL, 0.86 mmol) in dichloromethane (2 mL) at 0° C. was added methanesulfonyl chloride (89 mg, 60 μL, 0.78 mmol). The reaction mixture was stirred at room temperature for 2.5 h. A second portion of methanesulfonyl chloride (88 mg, 60 μL, 0.78 mmol) and triethylamine (87 mg, 0.12 mL, 0.86 mmol) were added at room temperature and the reaction was stirred at room temperature for 66 h. The reaction mixture was diluted with dichloromethane (20 mL) and the organic layer was washed with water (10 mL), saturated aqueous solution of sodium bicarbonate (10 mL) and brine (10 mL), dried over anhydrous sodium sulfate, filtered and concentrated under reduced pressure. The crude product was purified by silica gel flash chromatography using a gradient from 0 to 10% EtOAc in heptanes to afford 1-chloro-2-(1-chloro-2,2,2-trifluoro-1-methyl-ethyl)-5-methoxy-4-methyl-benzene (61 mg, 81%) as a yellow oil. ¹H NMR (400 MHz, CDCl₃) δ 7.50 (s, 1H), 6.87 (s, 1H), 3.84 (s, 3H), 2.34 (d, J=0.7 Hz, 3H), 2.20 (s, 3H). ¹⁹F NMR (377 MHz, CDCl₃) δ −74.67 (s, 3F).

### Step 3: 2-(2-chloro-4-methoxy-5-methyl-phenyl)-3,3,3-trifluoro-2-methyl-propanenitrile

To a solution of 1-chloro-2-(1-chloro-2,2,2-trifluoro-1-methyl-ethyl)-5-methoxy-4-methyl-benzene (6 g, 20.3 mmol) in 1,2-dichloroethane (120 mL) at room temperature was added trimethylsilyl cyanide (4.36 g, 5.5 mL, 44 mmol) followed by titanium tetrachloride in dichloromethane (23.5 mL of 1 M, 23 mmol) and the solution was stirred at 50° C. for 5 h. The reaction mixture was cooled to room temperature and slowly quenched with water (750 mL), diluted with DCM (1 L) and filtered through a pad of Celite. The layers were separated and the aqueous layer was extracted with DCM (350 mL). The combined organic layers were washed with brine (500 mL), dried over anhydrous sodium sulfate, filtered and concentrated under reduced pressure to give crude 2-(2-chloro-4-methoxy-5-methyl-phenyl)-3,3,3-trifluoro-2-methyl-propanenitrile (5.96 g, 90%) as a yellow oil. ESI-MS m/z calc. 277.0481, found 278.1 (M+1)⁺. ¹H NMR (400 MHz, CDCl₃) δ 7.28 (overlapped with chloroform, s, 1H), 6.91 (s, 1H), 3.86 (s, 3H), 2.21 (s, 3H), 2.14 (s, 3H). ¹⁹F NMR (377 MHz, CDCl₃) δ −71.94 (s, 3F).

### Step 4: 2-(2-chloro-4-hydroxy-5-methyl-phenyl)-3,3,3-trifluoro-2-methyl-propanenitrile

A solution of boron tribromide in DCM (29 mL of 1 M, 29 mmol) was added dropwise to a solution of 2-(2-chloro-4-methoxy-5-methyl-phenyl)-3,3,3-trifluoro-2-methyl-propanenitrile (2 g, 5.76 mmol) in DCM (50 mL) at −78° C. over 15 minutes. The reaction mixture was then gradually warmed up to room temperature and stirred for 24 h. The reaction mixture was quenched with ice water (500 mL) and diluted with DCM (500 mL). The layers were separated, the organic layer was dried over anhydrous sodium sulfate, filtered and concentrated under reduced pressure. The crude product was purified by silica gel flash chromatography using 0 to 40% EtOAc in heptanes to afford 2-(2-chloro-4-hydroxy-5-methyl-phenyl)-3,3,3-trifluoro-2-methyl-propanenitrile (1.36 g, 87%) as a tan solid. ESI-MS m/z calc. 263.03, found 264.1 (M+1)⁺. ¹H NMR (400 MHz, DMSO-d₆) δ 10.38 (s, 1H), 7.42 (s, 1H), 6.95 (s, 1H), 2.14 (s, 6H). ¹⁹F NMR (377 MHz, DMSO-d₆) δ −71.84 (s, 3F).

### Step 5: 2-(4-benzyloxy-2-chloro-5-methyl-phenyl)-3,3,3-trifluoro-2-methyl-propanenitrile

To a mixture of 2-(2-chloro-4-hydroxy-5-methyl-phenyl)-3,3,3-trifluoro-2-methyl-propanenitrile (115 mg, 0.42 mmol) and grinded potassium carbonate (118 mg, 0.86 mmol) in DMF (2 mL) at 0° C. was added bromomethylbenzene (1.29 g, 0.9 mL, 7.7 mmol). The cold bath was removed and the reaction mixture was stirred at room temperature for 2 h. The reaction mixture was diluted with water (50 mL) and poured into a separatory funnel. The mixture was extracted with EtOAc (2×50 mL). The combined organic layers were washed with water (2×100 mL), brine (100 mL), dried over anhydrous magnesium sulfate, filtered and concentrated under reduced pressure. The crude product was purified by silica gel flash chromatography using a gradient from 0 to 60% EtOAc in heptanes to afford 2-(4-benzyloxy-2-chloro-5-methyl-phenyl)-3,3,3-trifluoro-2-methyl-propanenitrile (106 mg, 67%) as an orange solid. ESI-MS m/z calc. 353.0794, found 354.1 (M+1)⁺. ¹H NMR (400 MHz, CDCl₃) δ 7.46-7.35 (m, 5H), 7.31 (s, 1H), 6.99 (s, 1H), 5.10 (s, 2H), 2.27 (s, 3H), 2.15 (s, 3H). ¹⁹F NMR (377 MHz, CDCl₃) δ −71.88 (s, 3F).

### Step 6: tert-butyl-[2-[2-chloro-5-methyl-4-(4,4,5,5-tetramethyl-1,3,2-dioxaborolan-2-yl)phenyl]-3,3,3-trifluoro-2-methyl-propoxy]-dimethyl-silane (Intermediate B-93)

tert-butyl-[2-[2-chloro-5-methyl-4-(4,4,5,5-tetramethyl-1,3,2-dioxaborolan-2-yl)phenyl]-3,3,3-trifluoro-2-methyl-propoxy]-dimethyl-silane (Intermediate B-93, 58 mg, 100%) was prepared from 2-(4-benzyloxy-2-chloro-5-methyl-phenyl)-3,3,3-trifluoro-2-methyl-propanenitrile using procedure analogous to that found in Intermediate B-90 (Step 3 to Step 7) as an orange oil. ¹H NMR (400 MHz, CDCl₃) δ 7.72 (s, 1H), 7.32 (s, 1H), 4.43 (d, J=11.2 Hz, 1H), 4.16 (d, J=10.4 Hz, 1H), 2.48 (s, 3H), 1.73 (s, 3H), 1.33 (s, 12H), 0.78 (s, 9H), 0.02 (s, 3H), 0.01 (s, 3H). ¹⁹F NMR (377 MHz, CDCl₃) δ −69.94 (s, 3F).

### Intermediate B-94

### 2-(4-tert-butyl-3,5-difluoro-2-methyl-phenyl)-4,4,5,5-tetramethyl-1,3,2-dioxaborolane

### Step 1: 4-tert-butyl-3,5-difluoro-phenol

To a 2-methoxy-2-methyl-propane (3 mL, 25.22 mmol) solution of 3,5-difluorophenol (1 g, 7.69 mmol), tetrachlorozirconium (900 mg, 3.86 mmol) was added slowly by keeping the reaction mixture at 30-40° C. After stirring at room temperature for 2 hours, additional tetrachlorozirconium (900 mg, 3.86 mmol) was slowly added and the reaction mixture was stirred for 16 hours at room temperature. The reaction mixture was poured into ice and 1N aqueous solution of sodium hydroxide (20 mL). DCM was added and the insoluble substances were separated by filtration. The filtrate was washed with brine, dried over magnesium sulfate and the solvent was evaporated under reduced pressure. The resulting residue was purified by silica gel column chromatography using 0 to 5% EtOAc in hexanes to obtain 4-tert-butyl-3,5-difluoro-phenol (250 mg, 17%). ¹H NMR (400 MHz, CDCl₃) δ 6.37-6.26 (m, 2H), 4.97 (s, 1H), 1.42 (t, J=2.3 Hz, 9H).

### Step 2: 2-bromo-4-tert-butyl-3,5-difluoro-phenol

To a suspension of 4-tert-butyl-3,5-difluoro-phenol (450 mg, 2.417 mmol) in ACN (5 mL) was added 4-methylbenzenesulfonic acid monohydrate (50 mg, 0.26 mmol) and the reaction mixture was cooled to 0° C. NBS (390 mg, 2.191 mmol) was added and the reaction mixture was stirred at room temperature for 18 hours. The reaction mixture was quenched with saturated aqueous solution of sodium bisulfite. The aqueous layer was extracted with EtOAc (2×), dried over magnesium sulfate, filtered and concentrated. Purification by silica gel column chromatography using hexanes gave 2-bromo-4-tert-butyl-3,5-difluoro-phenol (370 mg, 58%). ¹H NMR (400 MHz, CDCl₃) δ 6.56 (dd, J=13.8, 2.2 Hz, 1H), 5.55 (d, J=1.4 Hz, 1H), 1.44 (t, J=2.3 Hz, 9H). ¹⁹F NMR (376 MHz, CDCl₃) δ −98.44 (tt, J=5.3, 3.0 Hz), −105.64-105.84 (m).

### Step 3: 4-bromo-2-tert-butyl-1,3-difluoro-5-methoxy-benzene

To a solution of 2-bromo-4-tert-butyl-3,5-difluoro-phenol (365 mg, 1.38 mmol) in DMF (3 mL) was added potassium carbonate (385 mg, 2.79 mmol) and iodomethane (100 μL, 1.61 mmol) and the reaction mixture was stirred at room temperature for 18 hours. The reaction mixture was quenched with water and the aqueous layer was extracted with DCM (3×). The combined organic layer was dried over magnesium sulfate, filtered and concentrated. Purified via silica gel column chromatography using hexanes to obtain 4-bromo-2-tert-butyl-1,3-difluoro-5-methoxy-benzene (321 mg, 84%). ¹H NMR (400 MHz, CDCl₃) δ 6.42 (dd, J=14.5, 2.1 Hz, 1H), 3.86 (s, 3H), 1.44 (t, J=2.3 Hz, 9H). ¹⁹F NMR (376 MHz, CDCl₃) δ −97.28-−97.66 (m), −105.43-−105.79 (m).

### Step 4: 2-tert-butyl-1,3-difluoro-5-methoxy-4-methyl-benzene

A microwave vial charged with 4-bromo-2-tert-butyl-1,3-difluoro-5-methoxy-benzene (315 mg, 1.13 mmol), 2,4,6-trimethyl-1,3,5,2,4,6-trioxatriborinane (430 μL of 50% w/v, 1.71 mmol), Pd(dppf)Cl₂·DCM (95 mg, 0.11 mmol), tripotassium phosphate (710 mg, 3.34 mmol), water (300 μL) and dioxane (3 mL) was degassed under nitrogen, sealed and heated at 100° C. for 18 hours. The reaction mixture was filtered and concentrated. Purification via silica gel column chromatography using 0 to 5% EtOAc in hexanes gave 2-tert-butyl-1,3-difluoro-5-methoxy-4-methyl-benzene (190 mg, 79%). ¹H NMR (400 MHz, CDCl₃) δ 6.31 (dd, J=14.9, 2.0 Hz, 1H), 3.77 (s, 3H), 2.04 (dd, J=3.0, 1.1 Hz, 3H), 1.43 (t, J=2.3 Hz, 9H).

### Step 5: 4-tert-butyl-3,5-difluoro-2-methyl-phenol

To a solution of 2-tert-butyl-1,3-difluoro-5-methoxy-4-methyl-benzene (185 mg, 0.86 mmol) in DCM (2 mL) at −70° C. was added tribromoborane in DCM (1.4 mL of 1 M, 1.4 mmol). After 5 min, the dry-ice bathe was removed and the reaction mixture was stirred at room temperature for 16 h. The reaction mixture was slowly quenched with methanol. The solvent was removed under reduced pressure and purification via silica gel column chromatography using 0 to 10% EtOAc in hexanes gave 4-tert-butyl-3,5-difluoro-2-methyl-phenol (140 mg, 81%). ESI-MS m/z calc. 200.10, found 200.76 (M+1)⁺. ¹H NMR (400 MHz, CDCl₃) δ 6.29 (dd, J=13.7, 2.1 Hz, 1H), 4.78 (s, 1H), 2.07 (dd, J=2.7, 1.1 Hz, 3H), 1.42 (t, J=2.3 Hz, 9H).

### Step 6: 2-(4-tert-butyl-3,5-difluoro-2-methyl-phenyl)-4,4,5,5-tetramethyl-1,3,2-dioxaborolane (Intermediate B-94)

2-(4-tert-butyl-3,5-difluoro-2-methyl-phenyl)-4,4,5,5-tetramethyl-1,3,2-dioxaborolane (Intermediate B-94) was prepared from 4-tert-butyl-3,5-difluoro-2-methyl-phenol, using procedure analogous to that found in Intermediate B-24 (Step 1). ESI-MS m/z calc. 310.19, found 311.3 (M+1)⁺. ¹H NMR (400 MHz, CDCl₃) δ 7.17 (dd, J=13.3, 1.7 Hz, 1H), 2.37 (dd, J=3.6, 1.2 Hz, 3H), 1.45 (t, J=2.3 Hz, 9H), 1.33 (s, 12H).

### Intermediate B-95

### 2-(4-tert-butyl-5-chloro-3-fluoro-2-methyl-phenyl)-4,4,5,5-tetramethyl-1,3,2-dioxaborolane

### Step 1: 2-bromo-5-chloro-1-fluoro-3-methoxy-benzene

To a solution of 2-bromo-5-chloro-3-fluoro-phenol (5 g, 22.18 mmol) in DMF (45 mL) was added potassium carbonate (6.25 g, 45.22 mmol) and iodomethane (1.7 mL, 27.31 mmol) and the reaction mixture was stirred at room temperature for 18 hours. The reaction mixture was quenched with water and the aqueous layer was extracted with EtOAc (3×). The combined organic layers were washed with brine (2×), dried over MgSO₄, filtered and concentrated. Purified via silica gel column chromatography using 100% hexanes to obtain 2-bromo-5-chloro-1-fluoro-3-methoxy-benzene (4.90 g, 92%). ¹H NMR (400 MHz, CDCl₃) δ 6.81 (dd, J=8.0, 2.2 Hz, 1H), 6.70 (t, J=2.0 Hz, 1H), 3.91 (s, 3H).

### Step 2: 5-chloro-1-fluoro-3-methoxy-2-methyl-benzene

A microwave vial charged with 2-bromo-5-chloro-1-fluoro-3-methoxy-benzene (1.58 g, 6.6 mmol), 2,4,6-trimethyl-1,3,5,2,4,6-trioxatriborinane (2.5 mL of 50% w/v, 9.96 mmol), Pd(dppf)Cl₂ DCM (555 mg, 0.7 mmol), tripotassium phosphate (4.1 g, 19.32 mmol), water (1.5 mL), dioxane (15 mL) and water (1.5 mL) and was degassed under nitrogen, sealed and heated at 100° C. for 18 hours. The reaction mixture was filtered and concentrated. Purification via silica gel column chromatography using 0 to 5% EtOAc in hexanes gave 5-chloro-1-fluoro-3-methoxy-2-methyl-benzene (835 mg, 43%) ¹H NMR (400 MHz, CDCl₃) δ 6.70 (dd, J=8.9, 1.9 Hz, 1H), 6.62 (t, J=1.7 Hz, 1H), 3.82 (s, 3H), 2.07 (s, 3H).

### Step 3: 5-chloro-3-fluoro-2-methyl-phenol

To a solution of 5-chloro-1-fluoro-3-methoxy-2-methyl-benzene (2.16 g, 12.37 mmol) in anhydrous DCM (35 mL) at room temperature, was added tribromoborane (15 mL of 1 M, 15 mmol) dropwise. The reaction mixture was stirred for 22 h, cooled to −78° C. and quenched by careful dropwise addition of water. The resulting suspension was diluted with water, extracted with DCM, dried over sodium sulfate, filtered and concentrated under reduced pressure. Purification by flash chromatography on silica gel using 0 to 20% EtOAc in hexanes provided 5-chloro-3-fluoro-2-methyl-phenol (1.96 g, 99%). H NMR (400 MHz, CDCl₃) δ 6.68 (dd, J=9.0, 2.0 Hz, 1H), 6.61 (t, J=1.8 Hz, 1H), 4.85 (d, J=1.3 Hz, 1H), 2.12 (d, J=1.7 Hz, 3H). ¹⁹F NMR (377 MHz, CDCl₃) δ− 114.21 (s, 1F).

### Step 4: 4-bromo-5-chloro-3-fluoro-2-methyl-phenol

To a suspension of 5-chloro-3-fluoro-2-methyl-phenol (1.96 g, 12.21 mmol) and 4-methylbenzenesulfonic acid monohydrate (275 mg, 1.45 mmol) in ACN (22 mL) at 0° C., was added NBS (2.2 g, 12.36 mmol). The reaction mixture was stirred at room temperature for 18 hours, quenched with water and the aqueous layer extracted with EtOAc (2×). The combined organic layers were dried over magnesium sulfate, filtered and concentrated. Purification by silica gel column chromatography using 0 to 5% EtOAc in hexanes gave 4-bromo-5-chloro-3-fluoro-2-methyl-phenol (1.870 g, 61%). ¹H NMR (400 MHz, CDCl₃) δ 6.78 (d, J=1.9 Hz, 1H), 5.00 (d, J=1.3 Hz, 1H), 2.17 (d, J=2.1 Hz, 3H).

### Step 5: 2-bromo-1-chloro-3-fluoro-5-methoxy-4-methyl-benzene

To a solution of 4-bromo-5-chloro-3-fluoro-2-methyl-phenol (315 mg, 1.315 mmol) in DMF (3 mL) was added potassium carbonate (367 mg, 2.65 mmol) and iodomethane (100 μL, 1.60 mmol) and the reaction mixture was stirred at room temperature for 18 hours. The reaction mixture was quenched with water and the aqueous layer was extracted with EtOAc (3×). The combined organic layer was washed with brine (2×), dried over magnesium sulfate, filtered and concentrated. Purified via silica gel column chromatography using hexanes gave 2-bromo-1-chloro-3-fluoro-5-methoxy-4-methyl-benzene (295 mg, 89%). ¹H NMR (400 MHz, CDCl₃) δ 6.77 (d, J=1.8 Hz, 1H), 3.82 (s, 3H), 2.13 (d, J=2.3 Hz, 3H). ¹⁹F NMR (376 MHz, CDCl₃) δ −103.63-−103.68 (m).

### Step 6: 1-(6-chloro-2-fluoro-4-methoxy-3-methyl-phenyl)ethanone

To a vial containing a solution of 2-bromo-1-chloro-3-fluoro-5-methoxy-4-methyl-benzene (290 mg, 1.144 mmol) and tributyl(1-ethoxyvinyl)stannane (400 μL, 1.18 mmol) in dioxane (1.5 mL) was added dichloropalladium;triphenylphosphane (40 mg, 0.06 mmol). The reaction mixture was degassed with nitrogen for 30-60 seconds, sealed and was heated at 100° C. for 17 hours. The reaction mixture cooled to room temperature and quenched with water. The aqueous layer was extracted with DCM (3×). The combined organic layer was dried over magnesium sulfate, filtered and concentrated. The obtained material was taken up in THF (2 mL) and HCl in water (2 mL of 1 M, 2 mmol) was added and the reaction mixture was stirred at room temperature for 1 hour, The aqueous layer was extracted with DCM (3×), dried over MgSO₄, filtered and concentrated. The crude material was purified via silica gel column chromatography using 0 to 15% EtOAc in hexanes to obtain 1-(6-chloro-2-fluoro-4-methoxy-3-methyl-phenyl)ethanone (131 mg, 53%). ESI-MS m/z calc. 216.03, found 217.1 (M+1)⁺. ¹H NMR (400 MHz, CDCl₃) δ 6.67 (d, J=1.6 Hz, 1H), 3.85 (s, 3H), 2.60-2.53 (m, 3H), 2.09 (d, J=2.2 Hz, 3H). ¹⁹F NMR (376 MHz, CDCl₃) δ −116.18-116.26 (m).

### Step 7: 2-tert-butyl-1-chloro-3-fluoro-5-methoxy-4-methyl-benzene

To a solution of tetrachlorotitanium in toluene (1.5 mL of 1 M, 1.5 mmol) in DCM (1 mL) at −40° C. was added dimethylzinc in heptane (2 mL of 1 M, 2 mmol) slowly maintaining the temperature below −40° C. (internal temperature). The reaction mixture was stirred at −40° C. for 30 min and then a solution of 1-(6-chloro-2-fluoro-4-methoxy-3-methyl-phenyl)ethanone (130 mg, 0.6 mmol) in DCM (300 μL) was added dropwise, maintaining the internal temperature below −40° C. The reaction mixture was gradually warmed to room temperature and stirred for 16 hours. The reaction mixture was quenched by slowly pouring it into an ice/saturated sodium bicarbonate solution. The aqueous phase was acidified with conc. HCl and then extracted with DCM. The organic layer was combined, dried over magnesium sulfate, filtered, and concentrated in vacuo. The crude material was purified via silica gel column chromatography using hexanes to obtain 2-tert-butyl-1-chloro-3-fluoro-5-methoxy-4-methyl-benzene (72 mg, 52%) ¹H NMR (400 MHz, CDCl₃) δ 6.63 (d, J=1.9 Hz, 1H), 3.79 (s, 3H), 2.05 (d, J=3.1 Hz, 3H), 1.54 (d, J=3.3 Hz, 9H).

### Step 8: 2-(4-tert-butyl-5-chloro-3-fluoro-2-methyl-phenyl)-4,4,5,5-tetramethyl-1,3,2-dioxaborolane (Intermediate B-95)

2-(4-tert-butyl-5-chloro-3-fluoro-2-methyl-phenyl)-4,4,5,5-tetramethyl-1,3,2-dioxaborolane (Intermediate B-95) was prepared form 2-tert-butyl-1-chloro-3-fluoro-5-methoxy-4-methyl-benzene, using procedure analogous to that found in Intermediate B-94 (Step 5 and Step 6). ¹H NMR (400 MHz, CDCl₃) δ 7.50 (s, 1H), 2.38 (d, J=3.9 Hz, 3H), 1.56 (d, J=3.5 Hz, 9H), 1.33 (s, 12H).

### Intermediate B-96

### 2-(4-tert-butyl-3-chloro-2-fluoro-6-methyl-phenyl)-4,4,5,5-tetramethyl-1,3,2-dioxaborolane

### Step 1: 4-tert-butyl-3-chloro-2-fluoro-phenol

To a solution of 3-chloro-2-fluoro-phenol (10 g, 68.24 mmol) in tert-butanol (20 mL, 209.1 mmol) and heptane (80 mL) in a round bottom flask was added sulfuric acid (7.3 mL, 137 mmol) and the reaction mixture was stirred for 10 days. The reaction mixture was poured onto ice and diluted with water. The aqueous layer was extracted with EtOAc (3×). The combined organic layer was washed with sat. Na₂S₂O₃ solution, water, dried over magnesium sulfate, filtered and concentrated. Purified via silica gel column chromatography using 0 to 5% EtOAc in hexanes followed by purification by reverse phase column chromatography (C₁₈) using 1 to 99% ACN in water (HCl modifier) gave 4-tert-butyl-3-chloro-2-fluoro-phenol (1.2 g, 9%). ¹H NMR (400 MHz, CDCl₃) δ 7.07 (dd, J=8.9, 2.1 Hz, 1H), 6.84 (t, J=8.8 Hz, 1H), 5.10 (s, 1H), 1.45 (s, 9H).

### Step 2: 6-bromo-4-tert-butyl-3-chloro-2-fluoro-phenol

To a solution of 4-tert-butyl-3-chloro-2-fluoro-phenol (1.2 g, 5.921 mmol) in acetonitrile (12 mL) was added NBS (2.15 g, 12.08 mmol) and the reaction was stirred at room temperature for 18 hours. The reaction mixture was quenched with water and the layers were separated. The aqueous layer was extracted with DCM (2×). The combined organic layer was washed with sat. sodium sulfite solution, followed by washing with water, dried over magnesium sulfate, filtered and concentrated under reduced pressure. The crude product was purified by silica gel column chromatography using 0 to 5% ethyl acetate in hexanes to afford 6-bromo-4-tert-butyl-3-chloro-2-fluoro-phenol (1.5 g, 90%). ¹H NMR (400 MHz, CDCl₃) δ 7.30 (d, J=2.3 Hz, 1H), 5.48 (s, 1H), 1.45 (s, 9H).

### Step 3: (6-bromo-4-tert-butyl-3-chloro-2-fluoro-phenyl) methyl carbonate

6-bromo-4-tert-butyl-3-chloro-2-fluoro-phenol (1.5 g, 5.33 mmol) and DMAP (35 mg, 0.28 mmol) were dissolved in DCM (8 mL) and TEA (1.5 mL, 10.76 mmol), cooled to 0° C., then treated with methylchloroformate (620 μL, 8.02 mmol). The reaction mixture was gradually warmed to room temperature and stirred for 2 h. The reaction was quenched with water, the layers separated, and the aqueous layer was extracted via DCM. The combined organic layer was dried over anhydrous magnesium sulfate, filtered and concentrated. Purification by silica gel column chromatography using 0 to 5% EtOAc in hexane gave (6-bromo-4-tert-butyl-3-chloro-2-fluoro-phenyl) methyl carbonate (1.54 g, 85%). ESI-MS m/z calc. 337.97, found 341.1 (M+3)⁺. H NMR (400 MHz, CDCl₃) δ 7.43 (d, J=2.3 Hz, 1H), 3.98 (s, 3H), 1.48 (s, 9H).

### Step 4: 4-tert-butyl-3-chloro-2-fluoro-6-methyl-phenol

A microwave vial charged with (6-bromo-4-tert-butyl-3-chloro-2-fluoro-phenyl) methyl carbonate (700 mg, 2.06 mmol), 2,4,6-trimethyl-1,3,5,2,4,6-trioxatriborinane (770 μL of 50% w/v, 3.07 mmol), Pd(dppf)Cl₂·DCM (170 mg, 0.2 mmol), tripotassium phosphate (1.3 g, 6.12 mmol), water (700.0 μL) and dioxane (7 mL) was degassed under nitrogen, sealed and heated at 100° C. for 18 hours. The reaction mixture was filtered and concentrated. Purification via silica gel column chromatography using 0 to 5% EtOAc in hexanes gave 4-tert-butyl-3-chloro-2-fluoro-6-methyl-phenol (309 mg, 69%). ¹H NMR (400 MHz, CDCl₃) δ 6.96-6.92 (m, 1H), 5.08 (d, J=5.0 Hz, 1H), 2.24 (d, J=0.7 Hz, 3H), 1.44 (s, 9H).

### Step 5: 2-(4-tert-butyl-3-chloro-2-fluoro-6-methyl-phenyl)-4,4,5,5-tetramethyl-1,3,2-dioxaborolane (Intermediate B-96)

2-(4-tert-butyl-3-chloro-2-fluoro-6-methyl-phenyl)-4,4,5,5-tetramethyl-1,3,2-dioxaborolane (Intermediate B-96, 100 mg, 22%) was prepared from 4-tert-butyl-3-chloro-2-fluoro-6-methyl-phenol, using procedure analogous to that found in Intermediate B-24 (Step 1 and Step 2). ESI-MS m/z calc. 326.16, found 327.3 (M+1)⁺. ¹H NMR (400 MHz, CDCl₃) δ 6.98 (s, 1H), 2.40 (s, 3H), 1.45 (s, 9H), 1.37 (s, 12H).

### Intermediate B-97

### 2-(4-tert-butyl-5-fluoro-2-methyl-phenyl)-4,4,5,5-tetramethyl-1,3,2-dioxaborolane

### Step 1: 2-(4-tert-butyl-5-fluoro-2-methyl-phenyl)-4,4,5,5-tetramethyl-1,3,2-dioxaborolane (Intermediate B-97)

2-(4-tert-butyl-5-fluoro-2-methyl-phenyl)-4,4,5,5-tetramethyl-1,3,2-dioxaborolane (Intermediate B-97) was prepared from 5-fluoro-2-methyl-phenol, using procedure analogous to that found in Intermediate B-32 (Step 1 and Step 3) ¹H NMR (400 MHz, CDCl₃) δ 7.36 (d, J=13.3 Hz, 1H), 7.06 (d, J=7.9 Hz, 1H), 2.48 (s, 3H), 1.38-1.30 (m, 21H).

### Intermediate B-98

### 2-(4-tert-butyl-2,3-difluoro-6-methyl-phenyl)-4,4,5,5-tetramethyl-1,3,2-dioxaborolane

### Step 1: 2-(4-tert-butyl-2,3-difluoro-6-methyl-phenyl)-4,4,5,5-tetramethyl-1,3,2-dioxaborolane (Intermediate B-98)

2-(4-tert-butyl-2,3-difluoro-6-methyl-phenyl)-4,4,5,5-tetramethyl-1,3,2-dioxaborolane (Intermediate B-98) was prepared from 2,3-difluoro-6-methyl-phenol, using procedure analogous to that found in Intermediate B-32 (Step 1 and Step 3). ¹H NMR (400 MHz, CDCl₃) δ 6.80 (d, J=6.2 Hz, 1H), 2.39 (s, 3H), 1.37 (s, 9H), 1.26 (s, 12H).

### Intermediate B-99

### 2-[5-chloro-4-(3,3-difluoro-1-methyl-cyclopentyl)-2-methyl-phenyl]-4,4,5,5-tetramethyl-1,3,2-dioxaborolane

### Step 1: 2-(4-bromo-2-chloro-5-methyl-phenyl)pent-4-en-2-ol

To a solution of 1-(4-bromo-2-chloro-5-methyl-phenyl)ethanone (500 mg, 1.74 mmol) in an ice/water bath at about 0° C. was added allylmagnesium bromide in diethyl ether (2.6 mL of 1 M, 2.6 mmol) and the reaction was stirred for 2 hours at this temperature. The reaction mixture was slowly quenched with saturated aqueous ammonium chloride solution. The aqueous layer was extracted with diethyl ether (2×50 mL). The combined organic layer was washed with brine (50 mL), dried over sodium sulfate, filtered and concentrated under reduced pressure. Purification by silica gel chromatography using 0 to 20% EtOAc in heptane afforded 2-(4-bromo-2-chloro-5-methyl-phenyl)pent-4-en-2-ol (510 mg, 94%) as a colorless oil. ESI-MS m/z calc. 287.99, found 271.0 (M−17)⁺. ¹H NMR (400 MHz, CDCl₃) δ 7.58 (s, 1H), 7.53 (s, 1H), 5.61-5.46 (m, 1H), 5.22-5.08 (m, 2H), 3.21 (ddt, J 13.9, 6.4, 1.0 Hz, 1H), 2.59 (dd, J 14.2, 8.6 Hz, 1H), 2.43 (s, 1H), 2.38 (s, 3H), 1.67 (s, 3H).

### Step 2: 1-(1-allyl-1-methyl-but-3-enyl)-4-bromo-2-chloro-5-methyl-benzene

Indium bromide (950 mg, 2.68 mmol) and allyl(trimethyl)silane (928 mg, 1.3 mL, 8.12 mmol) were added to a solution of 2-(4-bromo-2-chloro-5-methyl-phenyl)pent-4-en-2-ol (1.65 g, 5.35 mmol) in dichloroethane (20 mL) and reaction mixture was stirred at room temperature for 1 hour. The reaction mixture was diluted with water (50 mL) and extracted using dichloromethane (3×30 mL). The combined organic layer was washed with brine (50 mL), dried over sodium sulfate, filtered and concentrated to afford 1-(1-allyl-1-methyl-but-3-enyl)-4-bromo-2-chloro-5-methyl-benzene (1.68 g, 100%), which was used as crude for the next step.

### Step 3: 1-bromo-5-chloro-2-methyl-4-(1-methylcyclopent-3-en-1-yl)benzene

A solution of 1-(1-allyl-1-methyl-but-3-enyl)-4-bromo-2-chloro-5-methyl-benzene (1.68 g, 5.35 mmol) in DCE (500 mL) was sparged with nitrogen gas for 10-15 minutes. To the solution was added Grubbs catalyst, 2nd generation (270 mg, 0.32 mmol) and the reaction mixture was stirred at 40° C. for 18 hours. The crude reaction mixture was concentrated under reduced pressure and the residue was dissolved in a mixture of heptanes and acetonitrile (100 mL) and layers were separated. Acetonitrile was back extracted with heptanes (2×25 mL). The combined organic layer was washed with acetonitrile (20 mL) and concentrated under reduced pressure to afford crude 1-bromo-5-chloro-2-methyl-4-(1-methylcyclopent-3-en-1-yl)benzene.

### Step 4: 3-(4-bromo-2-chloro-5-methyl-phenyl)-3-methyl-cyclopentanol

Boranedimethyl sulfide (208.26 mg, 0.26 mL, 2.74 mmol) was slowly added to a solution of 1-bromo-5-chloro-2-methyl-4-(1-methylcyclopent-3-en-1-yl)benzene (400 mg, 1.38 mmol) in dry tetrahydrofurane (5 mL) at 0° C. and reaction mixture was warmed to room temperature and stirred for 1 hour. The reaction mixture was again cooled to 0° C., treated with 2N aqueous sodium hydroxide (2.5 mL) and 30% hydrogen peroxide (2.5 mL), wared to room temperature and stirred for 2 hours. The reaction mixture was diluted with water (20 mL) and extracted using dichloromethane (3×20 mL). The combined organic layer was dried over sodium sulfate, filtered and concentrated under reduced pressure to afford crude 3-(4-bromo-2-chloro-5-methyl-phenyl)-3-methyl-cyclopentanol (438 mg, 94%) as a clear oil. ESI-MS m/z calc. 302.01, found 285.1 (M−17)⁺. ¹H NMR (400 MHz, CDCl₃) δ 7.57-7.50 (m, 1H), 7.24 (s, 0.5H), 7.17 (s, 0.5H), 4.57-4.48 (m, 0.5H), 4.48-4.38 (m, 0.5H), 2.56 (dd, J 13.4, 6.6 Hz, 0.5H), 2.46-2.39 (m, 0.5H), 2.37 (s, 3H), 2.35-2.27 (m, 0.5H), 2.23-1.96 (m, 3.5H), 1.84-1.68 (m, 2H), 1.53 (s, 1.5H), 1.34 (s, 1.5H).

### Step 5: 3-(4-bromo-2-chloro-5-methyl-phenyl)-3-methyl-cyclopentanone

Dess-martin periodinane (1.2 g, 2.83 mmol) was added to a solution of 3-(4-bromo-2-chloro-5-methyl-phenyl)-3-methyl-cyclopentanol (438 mg, 1.3 mmol) in dichloromethane (10 mL) at 0° C. and reaction mixture was stirred at room temperature for 2.5 hours. The reaction mixture was diluted with water (50 mL) and extracted using dichloromethane (3×50 mL). The combine organic layer was washed with water (20 mL), brine (20 mL), dried over sodium sulfate, filtered and concentrated under reduced pressure. The residue was purified by silica gel column chromatography using 0 to 5% of methanol in dichloromethane to afford 3-(4-bromo-2-chloro-5-methyl-phenyl)-3-methyl-cyclopentanone (291 mg, 74%) as a clear oil. ESI-MS m/z calc. 299.99, found 301.1 (M+1)⁺. ¹H NMR (400 MHz, CDCl₃) δ 7.58 (s, 1H), 7.18 (s, 1H), 2.89 (dd, J 18.0, 1.3 Hz, 1H), 2.62 (d, J 17.9 Hz, 1H), 2.55-2.36 (m, 7H), 1.47 (s, 3H).

### Step 6: 1-bromo-5-chloro-4-(3,3-difluoro-1-methyl-cyclopentyl)-2-methyl-benzene

DAST (615 mg, 0.5 mL, 3.81 mmol) was added to a solution of 3-(4-bromo-2-chloro-5-methyl-phenyl)-3-methyl-cyclopentanone (240 mg, 0.8 mmol) in dichloroethane (0.5 mL) and reaction mixture was stirred at 40° C. for 5 hours. Additional DAST (615 mg, 0.5 mL, 3.81 mmol) was added and reaction mixture was stirred overnight at 40° C. Additional DAST (615 mg, 0.5 mL, 3.81 mmol) was added and the reaction mixture was stirred at 40° C. for another 6 hours. Once cooled to room temperature, the reaction mixture was then slowly poured into a vigorously stirring mixture of saturated aqueous sodium bicarbonate solution (50 mL) and dichloromethane (30 mL). The layers were separated and the aqueous layer was extracted with dichloromethane (2×15 mL). The combined organic layer was dried over sodium sulfate, filtered and concentrated under reduced pressure. Purification by silica gel flash chromatography using a 0 to 10% EtOAc in heptanes gave 1-bromo-5-chloro-4-(3,3-difluoro-1-methyl-cyclopentyl)-2-methyl-benzene (158 mg, 61%) as a clear oil. ¹H NMR (400 MHz, CDCl₃) δ 7.56 (s, 1H), 7.13 (s, 1H), 2.85-2.71 (m, 1H), 2.60-2.43 (m, 1H), 2.39 (s, 3H), 2.37-2.21 (m, 4H), 1.48 (s, 3H). ¹⁹F NMR (377 MHz, CDCl₃) δ −84.57 (d, J 230.2 Hz, 1F), −85.85 (d, J 226.2 Hz, 1F).

### Step 7: 2-[5-chloro-4-(3,3-difluoro-1-methyl-cyclopentyl)-2-methyl-phenyl]-4,4,5,5-tetramethyl-1,3,2-dioxaborolane (Intermediate B-99)

2-[5-chloro-4-(3,3-difluoro-1-methyl-cyclopentyl)-2-methyl-phenyl]-4,4,5,5-tetramethyl-1,3,2-dioxaborolane (Intermediate B-99) was prepared from 1-bromo-5-chloro-4-(3,3-difluoro-1-methyl-cyclopentyl)-2-methyl-benzene using procedure analogous to that found in Intermediate B-1, Step 2. ESI-MS m/z calc. 370.17, found 371.2 (M+1)⁺. ¹H NMR (400 MHz, CDCl₃) δ 7.75 (s, 1H), 7.07 (s, 1H), 2.88-2.72 (m, 1H), 2.62-2.46 (m, 4H), 2.38-2.21 (m, 4H), 1.49 (s, 3H), 1.35 (s, 12H). ¹⁹F NMR (377 MHz, CDCl₃) δ −84.46 (d, J 227.5 Hz, 1F), −85.91 (d, J 233.0 Hz, 1F).

### Intermediate B-100

### 2-(5-chloro-2-methyl-4-(2-(methyl-d₃)propan-2-yl-1,1,1,3,3,3-d₆)phenyl-3-d)-4,4,5,5-tetramethyl-1,3,2-dioxaborolane

### Step 1: 5-chloro-2-methylphen-3,4-d₂-ol-d

A solution of 5-chloro-2-methylphenol (5.0 g, 35.1 mmol) in 1,4-dioxane (24 mL) and 97% D₂SO₄ (5.8 mL, 105.2 mmol) was stirred at 80° C. for 1.5 h. The reaction mixture was diluted with D₂O (20 mL) and extracted with dichloromethane (60 mL). The organic layer was washed with D₂O (20 mL) and the combined aqueous layers were extracted with DCM (10 mL×2). The combined organic layers were dried over Na₂SO₄, filtered, and concentrated. To increase the deuterium content further, the crude material was mixed with 1,4-dioxane (22 mL) and 97% D₂SO₄ (5.8 mL, 105.2 mmol) and stirred at 60° C. for 1.5 h. The reaction mixture was diluted in D₂O (20 mL) and extracted with dichloromethane (DCM, 60 mL). The organic layer was washed with D₂O (20 mL) and the combined aqueous layer was extracted with DCM (10 mL×2). The combined organic layers were dried overNa₂SO₄, filtered, and concentrated to obtain 5-chloro-2-methylphen-3,4-d₂-ol-d (5.6 g).

### Step 2: 5-chloro-2-methyl-4-(2-(methyl-d₃)propan-2-yl-1,1,1,3,3,3-d6)phen-3-d-ol-d

5-chloro-2-methylphen-3,4-d₂-ol-d (14.55 g, 100 mmol, 14% w/w dioxane) was dissolved in t-BuOH-d₁₀ (28.0 mL, 297 mmol) and n-heptane (146 mL) and cooled in an ice-water bath. At 7° C., 98% D₂SO₄ (11.0 mL, 198 mmol) was added resulting in a light-brown 2-layer system that was stirred at room temperature overnight. While cooling in a water bath D₂O (150 mL) was added and the mixture was diluted with EtOAc (250 mL). The phases were separated and the organic layer was washed with D₂O (90 mL×2) and 1 M DCl in D₂O (40 mL). The aqueous layer was extracted with EtOAc (50 mL), and the combined organic layers were dried over Na₂SO₄, filtered and concentrated. Purification by silica gel flash chromatography using 0 to 10% EtOAc in heptanes afforded 5-chloro-2-methyl-4-(2-(methyl-d₃)propan-2-yl-1,1,1,3,3,3-d₆)phen-3-d-ol-d (21. 8 g).

### Step 3: 2-(5-chloro-2-methyl-4-(2-(methyl-d₃)propan-2-yl-1,1,1,3,3,3-d₆)phenyl-3-d)-4,4,5,5-tetramethyl-1,3,2-dioxaborolane (Intermediate B-100)

2-(5-chloro-2-methyl-4-(2-(methyl-d₃)propan-2-yl-1,1,1,3,3,3-d₆)phenyl-3-d)-4,4,5,5-tetramethyl-1,3,2-dioxaborolane (Intermediate B-100) was prepared from 5-chloro-2-methyl-4-(2-(methyl-d₃)propan-2-yl-1,1,1,3,3,3-d₆)phen-3-d-ol-d, using procedure analogous to Intermediate B-26 (Step 4 and Step 5). ¹H NMR (300 MHz, CDCl₃) δ 7.21 (s, 1H), 2.51 (s, 3H), 1.35 (s, 12H).

### Intermediate B-101

### 2-(5-chloro-2-methyl-4-(1,1,1-trifluoro-2-methylpropan-2-yl-3-¹³C-3,3,3-d₃)phenyl-3,6-d₂)-4,4,5,5-tetramethyl-1,3,2-dioxaborolane (Intermediate B-101)

### Step 1: 1,4-dibromo-2-chloro-5-methylbenzene-3,6-d₂

A mixture of 1-chloro-4-methylbenzene-2,3,5,6-d₄ (30.0 g, 230 mmol), iron powder (1.26 g, 22.6 mmol) in chloroform (60 mL) were stirred in a 500 mL 3-neck round-bottom flask fitted with an addition funnel and condenser. The mixture was stirred and heated to 30° C. Bromine (28.2 mL, 551 mmol) was added dropwise over 1 hour. When the addition of bromine was complete, the temperature was increased to 45° C., and the reaction mixture was stirred for 2 hours. The reaction was cooled to room temperature, diluted with dichloromethane (400 mL), and washed with water (2×180 mL) and saturated NaCl (200 mL). The organic phase was dried over Na₂SO₄, filtered and concentrated. The crude material was recrystallized from EtOH (270 mL) and water (180 mL) to provide 1,4-dibromo-2-chloro-5-methylbenzene-3,6-d₂ (66.5 g). ¹H NMR (400 MHz, CDCl₃): δ 2.35 (s, 3H).

### Step 2: 1-(4-bromo-2-chloro-5-methylphenyl-3,6-d₂)-2,2,2-trifluoro-ethan-1-one

To a 1 L 3-neck round-bottom flask fitted with an addition funnel and thermometer was added a solution of 1,4-dibromo-2-chloro-5-methylbenzene-3,6-d₂ (30.0 g, 105 mmol) in diethyl ether (585 mL) and cooled to −78° C. under a N₂ atmosphere. n-BuLi (54.5 mL, 2.5 M in hexanes, 136 mmol) was added dropwise over 30 minutes, and the mixture was further stirred at −78° C. for 2 hours. A solution of N-methyl-N-methoxytrifluoroacetamide (21.4 g, 136 mmol) in diethyl ether (90 mL) was added dropwise over 30 minutes. The reaction was stirred at −78° C. for another 2 hours before quenching with aqueous 1M HCl (280 mL). The crude mixture was slowly warmed to room temperature and the aqueous phase was extracted with diethyl ether (200 mL). The combined organic layers were washed with saturated NaCl (300 mL), dried over Na₂SO₄, and concentrated under reduced pressure. The crude material was purified by flash silica gel column chromatography using 5% ethyl acetate in hexanes to afford 1-(4-bromo-2-chloro-5-methylphenyl-3,6-d₂)-2,2,2-trifluoro-ethan-1-one (24.0 g). ¹H NMR (400 MHz, CDCl₃): δ 2.45 (s, 3H). ¹⁹F NMR (376 MHz, CDCl₃): δ −72.80 (s).

### Step 3: 2-(4-bromo-2-chloro-5-methylphenyl-3,6-d₂)-1,1,1-trifluoro-propan-3-1³C-3,3,3-d₃-2-ol

A mixture of magnesium turnings (3.96 g, 163 mmol) and solid iodine (5 crystals) in diethyl ether (260 mL) were stirred in a 1-neck 500 mL round-bottom flask fitted with a condenser at room temperature. A solution of iodomethane-¹³C-d₃ (19.80 g, 135.7 mmol) in diethyl ether (40 mL) was added dropwise through the top of the condenser at a rate that maintained a steady reflux. The resulting grey reaction mixture was stirred for 2 hours at room temperature under a N₂ atmosphere. Separately, a solution of 1-(4-bromo-2-chloro-5-methylphenyl-3,6-d₂)-2,2,2-trifluoroethan-1-one (30.50 g, 100.5 mmol) in diethyl ether (300 mL) was added to a 1 L 3-neck round-bottom flask fitted with a thermometer, addition funnel, and magnetic stir bar, and cooled to −10° C. under a N₂ atmosphere. The above Grignard solution was transferred to the addition funnel via cannula, and then added dropwise over 30 minutes at −10° C. After the addition was complete, the reaction mixture was stirred for 1.5 hours while keeping the temperature between −5 to 0° C. The reaction mixture was quenched with saturated NH₄Cl (300 mL), and the phases were separated. The organic phase was washed with saturated NaCl (300 mL), dried over Na₂SO₄, and concentrated. The crude material was purified by flash silica gel column chromatography using 0 to 10% ethyl acetate in hexanes to afford 2-(4-bromo-2-chloro-5-methylphenyl-3,6-d₂)-1,1,1-trifluoro-propan-3-¹³C-3,3,3-d₃-2-ol. (31.0 g). ¹H NMR (400 MHz, CDCl₃): δ 3.73 (d, J=4.8 Hz, 1H), 2.37 (s, 3H). ¹⁹F NMR (377 MHz, CDCl₃); δ −79.63 (s).

### Step 4: 2-(5-chloro-2-methyl-4-(1,1,1-trifluoro-2-methylpropan-2-yl-3-¹³C-3,3,3-d₃)phenyl-3,6-d₂)-4,4,5,5-tetramethyl-1,3,2-dioxaborolane (Intermediate B-101)

2-(5-chloro-2-methyl-4-(1,1,1-trifluoro-2-methylpropan-2-yl-3-¹³C-3,3,3-d₃)phenyl-3,6-d₂)-4,4,5,5-tetramethyl-1,3,2-dioxaborolane (Intermediate B-101) was prepared from 2-(4-bromo-2-chloro-5-methylphenyl-3,6-d₂)-1,1,1-trifluoro-propan-3-¹³C-3,3,3-d₃-2-ol, using procedure analogous to that found in Intermediate B-3 (Step 3 to Step 5). ¹H NMR (400 MHz, CDCl₃): δ 2.49 (s, 3H), 1.74 (m, 3H), 1.33 (s, 12H). ¹⁹F NMR (376 MHz, CDCl₃): δ −73.77 (s).

### Intermediate B-102

### 2-(2-chloro-5-methyl-4-(4,4,5,5-tetramethyl-1,3,2-dioxaborolan-2-yl)phenyl)-2-(methyl-d₃)propan-1,1,3,3,3-d₅-1-ol (Intermediate B-102)

### Step 1: 2-(4-bromo-2-chloro-5-methylphenyl)propan-1,1,1,3,3,3-d₆-2-ol

To a solution of 1,4-dibromo-2-chloro-5-methylbenzene (87.0 g, 306 mmol) in Et₂O (2.0 L) at −78° C. was added a solution of n-BuLi in hexanes (2.5 M, 122 mL, 305 mmol) dropwise over 1 h. The mixture was stirred at −78° C. for 30 min. Acetone-d₆ (70 mL, 952 mmol) was added dropwise over 30 min. The reaction mixture was stirred at −78° C. for 60 min. Then saturated NH₄Cl (aq) (2.0 L) was added slowly, and the reaction mixture was allowed to warm to room temperature. The organic layer was separated, and the aqueous layer was extracted with EtOAc (1.0 L×2). The combined organic layers were dried over Na₂SO₄, filtered, concentrated, and purified by silica gel column chromatography using 6 to 20% EtOAc in hexanes to afford 2-(4-bromo-2-chloro-5-methylphenyl)propan-1,1,1,3,3,3-d₆-2-ol.

### Step 2: 2-(4-bromo-2-chloro-5-methylphenyl)-2-(methyl-d₃)propanenitrile-3,3,3-d₃

2-(4-bromo-2-chloro-5-methylphenyl)-2-(methyl-d₃)propanenitrile-3,3,3-d₃ was prepared from 2-(4-bromo-2-chloro-5-methylphenyl)propan-1,1,1,3,3,3-d₆-2-ol, using a procedure analogous to that found in Intermediate B-46 (Step 2).

### Step 3: 2-(4-bromo-2-chloro-5-methylphenyl)-2-(methyl-d₃)propanamide

To 2-(4-bromo-2-chloro-5-methylphenyl)-2-(methyl-d₃)propanenitrile-3,3,3-d₃ (36.4 g, 131 mmol) and acetic acid (150 mL) was added H₂SO₄ (150 mL). The mixture was stirred at room temperature for 2 min until all solid dissolved. Then H₂O (88 mL) was added over 1 min. The mixture was heated to 100° C. and stirred for 18 h. The reaction was cooled to rt, diluted with water (1.3 L) and extracted with EtOAc (500 mL×3). The combined extracts were washed with brine (500 mL×2), dried over Na₂SO₄, filtered and concentered in vacuo at 48° C. to ˜94 g. It was then concentrated in high vacuum for 30 min to give 2-(4-bromo-2-chloro-5-methylphenyl)-2-(methyl-d₃)propanamide-3,3,3-d₃ (36.9 g, 95%).

### Step 4: 2-(4-bromo-2-chloro-5-methylphenyl)-2-(methyl-d₃)propanoic-3,3,3-d₃ acid

To H₂SO₄ (210 mL) was added 2-(4-bromo-2-chloro-5-methylphenyl)-2-(methyl-d₃)propanamide-3,3,3-d₃ (30.0 g, 80.9 mmol). The mixture was stirred at rt for 1 h until all solid was dissolved. The resulting solution was cooled in an ice-water bath for 20 min. A solution of NaNO₂ (22.5 g, 326 mmol) in water (135 mL) was added dropwise over 20 min. The mixture was stirred at rt for 18 h. The foamy suspension was added to H₂O (1.8 L) and extracted with EtOAc (500 mL×3). The pooled extracts were washed with brine (500 mL), dried over Na₂SO₄, and concentrated. It was diluted with MTBE (600 mL) and extracted with 2N NaOH (130 mL×2). The combined extracts were washed with MTBE (500 mL). The aq. layer was adjusted to pH ˜1 with 3N HCl (˜220 mL). The mixture was extracted with MTBE (500 mL×3). The extracts were washed with brine (500 mL), dried over Na₂SO₄, and concentrated to give 2-(4-bromo-2-chloro-5-methylphenyl)-2-(methyl-d₃)propanoic-3,3,3-d₃ acid (23.8 g, 98% mmol).

### Step 5: 2-(2-chloro-5-methyl-4-(4,4,5,5-tetramethyl-1,3,2-dioxaborolan-2-yl)phenyl)-2-(methyl-d₃)propan-1,1,3,3,3-d₅-1-ol (Intermediate B-102)

2-(2-chloro-5-methyl-4-(4,4,5,5-tetramethyl-1,3,2-dioxaborolan-2-yl)phenyl)-2-(methyl-d₃)propan-1,1,3,3,3-d₅-1-ol (Intermediate B-102) was prepared from 2-(4-bromo-2-chloro-5-methylphenyl)-2-(methyl-d₃)propanoic-3,3,3-d₃ acid using procedure analogous to that found in Intermediate B-47 (Step 1 and Step 2). (BD₃.THF was used in Step 2 instead of BH₃.THF). ¹H NMR (400 MHz, CDCl₃) δ 7.72 (s, 1H), 7.21 (s, 1H), 2.49 (s, 3H), 1.33 (s, 12H).

### Example 3

### General Procedure for Suzuki Coupling of 2-chloroquinolin-4-ol with Intermediate B

Method—A

A microwave vial charged with Intermediate A (1 eq), Intermediate B (1-2 eq, custom or commercial boronic acid or boronic ester), XPhos Pd G3 (1-5 mol %), X-Phos (1-10 mol %), potassium carbonate (2-3 eq), ethanol and water was degassed under an atmosphere of nitrogen for 1-2 minutes. The vial was sealed and subjected to microwave irradiation at 100 to 120° C. for 30 minutes or heated thermally at 60 to 100° C. for 16 hours. The reaction mixture was filtered and purified via reverse phase HPLC (C₁₈) to obtain the desired products.

The following compounds (Table 2) were synthesized using the general Method A using commercially available boronic acid or boronic esters.

TABLE 2

| | | LC/MS | |
|---|---|---|---|
| | | (m/z | |
| | | calc.); | |
| Cmpd. | | Found | |
| No. | Compound Name | [M + H]⁺ | NMR (shifts in ppm) |
| 1 | 2-[3-fluoro-4- | 307.24 | |
| | (trifluoromethyl)phenyl]- | 308.1 | |
| | 1H-quinolin-4-one | | |
| 2 | 2-(4-methoxy-2,3- | 279.33 | |
| | dimethyl-phenyl)-1H- | 280.2 | |
| | quinolin-4-one | | |
| 3 | 2-indan-5-yl-1H-quinolin- | 261.32 | ¹H NMR (400 MHz, CD₃OD) δ (ppm) 8.29 |
| | 4-one | 262.14 | (dd, J = 8.2, 1.4 Hz, 1H), 7.86-7.75 (m, |
| | | | 2H), 7.68 (s, 1H), 7.58 (dd, J = 7.7, 1.8 Hz, |
| | | | 1H), 7.50 (ddd, J = 8.2, 6.7, 1.3 Hz, 1H), |
| | | | 7.43 (d, J = 7.9 Hz, 1H), 6.70 (s, 1H), 3.08- |
| | | | 2.98 (m, 4H), 2.16 (p, J = 7.5 Hz, 2H). |
| 4 | 2-(4-tert-butyl-2-methyl- | 291.39 | ¹H NMR (400 MHz, CD₃OD) δ (ppm) 8.29 |
| | phenyl)-1H-quinolin-4- | 292.3 | (dd, J = 8.3, 1.5 Hz, 1H), 7.73 (ddd, J = 8.4, |
| | one | | 6.9, 1.5 Hz, 1H), 7.61 (dt, J = 8.3, 0.9 Hz, |
| | | | 1H), 7.48-7.38 (m, 3H), 7.35 (d, J = 8.0 |
| | | | Hz, 1H), 6.29 (s, 1H), 2.35 (s, 3H), 1.36 (s, |
| | | | 9H). |
| 5 | 2-(4-tert-butylphenyl)-1H- | 277.36278 | ¹H NMR (400 MHz, CD₃OD) δ (ppm) 8.31- |
| | quinolin-4-one | .1 | 8.23 (m, 1H), 7.83-7.69 (m, 4H), 7.68- |
| | | | 7.59 (m, 2H), 7.44 (ddd, J = 8.2, 6.6, 1.5 Hz, |
| | | | 1H), 6.60 (s, 1H), 1.39 (s, 9H). |
| 6 | 2-(2,4-dimethylphenyl)- | 249.31 | |
| | 1H-quinolin-4-one | 250.1 | |
| 7 | 2-(5-tert-butyl-2-methyl- | 291.39 | ¹H NMR (400 MHz, CD₃OD) δ (ppm) 8.32 |
| | phenyl)-1H-quinolin-4- | 292.2 | (dd, J = 8.3, 1.4 Hz, 1H), 7.78 (ddd, J = 8.4, |
| | one | | 7.0, 1.5 Hz, 1H), 7.67 (dt, J = 8.3, 0.9 Hz, |
| | | | 1H), 7.55-7.47 (m, 2H), 7.44 (d, J = 2.1 |
| | | | Hz, 1H), 7.33 (d, J = 8.1 Hz, 1H), 6.37 (s, |
| | | | 1H), 2.30 (s, 3H), 1.35 (s, 9H). |
| 8 | 2-(4-cyclopropyl-2- | 275.34 | |
| | methyl-phenyl)-1H- | 276.16 | |
| | quinolin-4-one | | |
| 9 | 2-(4-cyclopropyl-2-fluoro- | 279.31 | ¹H NMR (400 MHz, CD₃OD) δ (ppm) 8.28 |
| | phenyl)-1H-quinolin-4- | 280.1 | (dd, J = 8.2, 1.4 Hz, 1H), 7.82-7.66 (m, |
| | one | | 2H), 7.55 (t, J = 7.9 Hz, 1H), 7.46 (ddd, J = |
| | | | 8.1, 6.8, 1.3 Hz, 1H), 7.11 (dd, J = 8.0, 1.7 |
| | | | Hz, 1H), 7.04 (dd, J = 12.2, 1.7 Hz, 1H), |
| | | | 6.48 (d, J = 1.1 Hz, 1H), 2.03 (tt, J = 8.6, 4.9 |
| | | | Hz, 1H), 1.30-1.01 (m, 2H), 0.81 (dt, J = |
| | | | 6.8, 4.6 Hz, 2H). |
| 10 | 2-[2-fluoro-4- | 307.24 | |
| | (trifluoromethyl)phenyl]- | 308.18 | |
| | 1H-quinolin-4-one | | |
| 11 | 2-(4-chloro-2-methoxy- | 285.72 | |
| | phenyl)-1H-quinolin-4- | 286.5 | |
| | one | | |
| 12 | 2-(4-methylindan-5-yl)- | 275.34 | ¹H NMR (500 MHz, DMSO-d₆) δ (ppm) |
| | 1H-quinolin-4-one | 276.5 | 12.11 (s, 1H), 8.15 (dd, J = 8.3, 3.6 Hz, 1H), |
| | | | 7.73-7.62 (m, 2H), 7.39 (ddt, J = 8.7, 5.6, |
| | | | 2.6 Hz, 1H), 7.22 (d, J = 2.9 Hz, 2H), 6.10 |
| | | | (d, J = 2.9 Hz, 1H), 2.95 (td, J = 7.5, 3.1 Hz, |
| | | | 2H), 2.88 (dt, J = 8.3, 5.4 Hz, 2H), 2.20 (d, |
| | | | J = 3.0 Hz, 3H), 2.19-2.01 (m, 2H). |
| 13 | 2-[2-methoxy-5- | 319.28 | |
| | (trifluoromethyl)phenyl]- | 320.23 | |
| | 1H-quinolin-4-one | | |
| 14 | 2-(4-tert-butyl-2-methoxy- | 307.39 | |
| | phenyl)-1H-quinolin-4- | 308.29 | |
| | one | | |
| 15 | 2-[2-methoxy-3- | 319.28 | |
| | (trifluoromethyl)phenyl]- | 320.29 | |
| | 1H-quinolin-4-one | | |
| 16 | 2-[3-methyl-4- | 303.28 | |
| | (trifluoromethyl)phenyl]- | 304.1 | |
| | 1H-quinolin-4-one | | |
| 17 | 2-[2-methyl-5- | 303.28 | |
| | (trifluoromethyl)phenyl]- | 304.1 | |
| | 1H-quinolin-4-one | | |
| 18 | 2-[2-hydroxy-4- | 305.25 | |
| | (trifluoromethyl)phenyl]- | 306.1 | |
| | 1H-quinolin-4-one | | |
| 19 | 2-[2-methoxy-4- | 319.28 | ¹H NMR (400 MHz, DMSO-d₆) δ (ppm) |
| | (trifluoromethyl)phenyl]- | 320.1 | 12.13 (s, 1H), 8.15 (s, 1H), 7.88-7.66 (m, |
| | 1H-quinolin-4-one | | 3H), 7.64-7.34 (m, 3H), 6.32 (s, 1H), 3.95 |
| | | | (s, 3H). |
| 20 | 2-[3-methyl-4- | 319.28 | ¹H NMR (400 MHz, DMSO-d₆) δ (ppm) |
| | (trifluoromethoxy)phenyl]- | 320.1 | 11.65 (s, 1H), 8.14-8.05 (m, 1H), 7.88 (s, |
| | 1H-quinolin-4-one | | 1H), 7.82-7.71 (m, 2H), 7.71-7.63 (m, |
| | | | 1H), 7.57-7.45 (m, 1H), 7.41-7.27 (m, |
| | | | 1H), 6.34 (s, 1H), 2.40 (s, 3H). |
| 21 | 2-[3-methyl-5- | 303.28 | ¹H NMR (400 MHz, DMSO-d₆) δ (ppm) |
| | (trifluoromethyl)phenyl]- | 304.1 | 12.54 (s, 1H), 8.29-8.14 (m, 1H), 8.04 (s, |
| | 1H-quinolin-4-one | | 2H), 7.98-7.87 (m, 1H), 7.87-7.71 (m, |
| | | | 2H), 7.61-7.40 (m, 1H), 6.70 (s, 1H), 2.53 |
| | | | (s, 3H). |
| 22 | 2-(2-methoxy-3-methyl- | 265.31 | ¹H NMR (400 MHz, DMSO-d₆) δ (ppm) |
| | phenyl)-1H-quinolin-4- | 266.2 | 11.83 (s, 1H), 8.19-8.04 (m, 1H), 7.74- |
| | one | | 7.59 (m, 2H), 7.51-7.39 (m, 1H), 7.36 (d, |
| | | | J = 7.5 Hz, 2H), 7.20 (t, J = 7.6 Hz, 1H), |
| | | | 6.26-6.09 (m, 1H), 3.53 (s, 3H), 2.34 (s, 3H). |
| 23 | 2-(4-methoxy-2,5- | 279.33280 | ¹H NMR (400 MHz, DMSO-d₆) δ (ppm) |
| | dimethyl-phenyl)-1H- | .1 | 11.87 (s, 1H), 8.21-8.02 (m, 1H), 7.78- |
| | quinolin-4-one | | 7.55 (m, 2H), 7.45-7.29 (m, 1H), 7.22 (s, |
| | | | 1H), 6.96 (s, 1H), 6.06 (s, 1H), 3.86 (s, 3H), |
| | | | 2.31 (s, 3H), 2.18 (s, 3H). |
| 24 | 2-(3-fluoro-2-methyl- | 253.27254 | ¹H NMR (400 MHz, DMSO-d₆) δ (ppm) |
| | phenyl)-1H-quinolin-4- | .2 | 11.73 (s, 1H), 8.21-8.05 (m, 1H), 7.69- |
| | one | | 7.62 (m, 1H), 7.62-7.56 (m, 1H), 7.41 (td, |
| | | | J = 7.9, 5.8 Hz, 1H), 7.38-7.33 (m, 1H), |
| | | | 7.33-7.25 (m, 1H), 6.36 (s, 1H), 5.99 (s, 1H), |
| | | | 2.21 (s, 3H). |
| 25 | 2-(3-methoxy-5-methyl- | 265.31 | |
| | phenyl)-1H-quinolin-4- | 266.2 | |
| | one | | |
| 26 | 2-(3-tert-butyl-5-methyl- | 291.39292 | ¹H NMR (400 MHz, DMSO-d₆) δ (ppm) |
| | phenyl)-1H-quinolin-4- | .2 | 11.53 (s, 1H), 8.14-8.07 (m, 1H), 7.81- |
| | one | | 7.73 (m, 1H), 7.66 (t, J = 8.0 Hz, 1H), 7.55 |
| | | | (s, 1H), 7.43 (d, J = 7.7 Hz, 2H), 7.32 (t, J = |
| | | | 7.7 Hz, 1H), 6.29 (s, 1H), 2.42 (s, 3H), 1.36 |
| | | | (d, J = 2.1 Hz, 9H). |
| 27 | 2-(3-ethoxy-2-fluoro-5- | 297.32 | |
| | methyl-phenyl)-1H- | 298.2 | |
| | quinolin-4-one | | |
| 28 | 2-[2-ethoxy-4- | 333.3 | |
| | (trifluoromethyl)phenyl]- | 334.3 | |
| | 1H-quinolin-4-one | | |
| 29 | 2-(4-chloro-3-ethyl- | 283.75 | |
| | phenyl)-1H-quinolin-4- | 284.1 | |
| | one | | |
| 30 | 2-[4-methoxy-3- | 319.28 | |
| | (trifluoromethyl)phenyl]- | 320.1 | |
| | 1H-quinolin-4-one | | |
| 31 | 2-(4-methoxy-3,5- | 279.33 | ¹H NMR (400 MHz, DMSO-d6) δ (ppm) |
| | dimethyl-phenyl)-1H- | 280.2 | 11.59 (s, 1H), 8.09 (d, J = 8.1, 1.5 Hz, 1H), |
| | quinolin-4-one | | 7.83-7.72 (m, 1H), 7.72-7.59 (m, 1H), |
| | | | 7.53 (s, 2H), 7.33 (t, J = 7.5 Hz, 1H), 6.32 |
| | | | (s, 1H), 3.73 (s, 3H), 2.33 (s, 6H). |
| 32 | 2-[3- | 305.25 | ¹H NMR (400 MHz, DMSO-d₆) δ (ppm) |
| | (trifluoromethoxy)phenyl]- | 306.1 | 11.69 (s, 1H), 8.22-8.04 (m, 1H), 7.94- |
| | 1H-quinolin-4-one | | 7.80 (m, 2H), 7.79-7.73 (m, 1H), 7.73- |
| | | | 7.63 (m, 2H), 7.63-7.51 (m, 1H), 7.35 (t, |
| | | | J = 7.5 Hz, 1H), 6.37 (s, 1H). |
| 33 | 2-(2-chloro-5-fluoro-4- | 287.72 | ¹H NMR (400 MHz, DMSO-d₆) δ (ppm) |
| | methyl-phenyl)-1H- | 288.1 | 11.83 (s, 1H), 8.12 (d, 1H), 7.72-7.64 (m, |
| | quinolin-4-one | | 1H), 7.64-7.55 (m, 2H), 7.54-7.48 (m, |
| | | | 1H), 7.35 (t, J = 7.4 Hz, 1H), 6.04 (s, 1H), |
| | | | 2.33 (s, 3H). |
| 34 | 2-12- | 305.25 | ¹H NMR (400 MHz, DMSO-d₆) δ (ppm) |
| | (trifluoromethoxy)phenyl]- | 306.1 | 12.08 (s, 1H), 8.14 (d, 1H), 7.76 (d, J = 7.9 |
| | 1H-quinolin-4-one | | Hz, 1H), 7.74-7.65 (m, 3H), 7.64-7.55 (m, |
| | | | 2H), 7.40 (t, J = 7.8 Hz, 1H), 6.20 (s, 1H). |
| 35 | 2-(2-methoxy-6-methyl- | 265.31 | ¹H NMR (400 MHz, DMSO-d₆) δ (ppm) |
| | phenyl)-1H-quinolin-4- | 266.2 | 11.77 (s, 1H), 8.12 (d, 1H), 7.67-7.60 (m, |
| | one | | 1H), 7.60-7.54 (m, 1H), 7.39 (t, J = 8.0 Hz, |
| | | | 1H), 7.36-7.29 (m, 1H), 7.02 (d, 1H), 6.97 |
| | | | (d, J = 7.6 Hz, 1H), 5.91 (s, 1H), 3.73 (s, |
| | | | 3H), 2.17 (s, 3H). |
| 36 | 2-(5-acetyl-2-methoxy- | 293.32 | ¹H NMR (400 MHz, DMSO-d₆) δ (ppm) |
| | phenyl)-1H-quinolin-4- | 294.2 | 12.04 (s, 1H), 8.15 (d, 2H), 8.08 (d, J = 2.2 |
| | one | | Hz, 1H), 7.74-7.66 (m, 2H), 7.42-7.37 (m, |
| | | | 1H), 7.35 (d, 1H), 6.28 (s, 1H), 3.93 (s, 3H), |
| | | | 2.59 (s, 3H). |
| 37 | 2-(4-tert-butoxyphenyl)- | 293.36 | |
| | 1H-quinolin-4-one | 294.2 | |
| 38 | 2-(2,4-diisopropylphenyl)- | 305.41 | |
| | 1H-quinolin-4-one | 306.3 | |
| 39 | 2-(2-tert-butylphenyl)-1H- | 277.36278 | |
| | quinolin-4-one | .2 | |
| 40 | 2-(2,3-dimethoxy-5- | 295.33 | |
| | methyl-phenyl)-1H- | 296.1 | |
| | quinolin-4-one | | |
| 41 | 2-[2-(hydroxymethyl)-5- | 319.28 | |
| | (trifluoromethyl)phenyl]- | 320.1 | |
| | 1H-quinolin-4-one | | |
| 42 | 2-(2,2-difluoro-1,3- | 301.24 | |
| | benzodioxol-4-yl)-1H- | 302.1 | |
| | quinolin-4-one | | |
| 43 | 2-indan-4-yl-1H-quinolin- | 261.32 | |
| | 4-one | 262.2 | |
| 44 | 2-(6-chloro-2-fluoro-3- | 287.72 | |
| | methyl-phenyl)-1H- | 288.2 | |
| | quinolin-4-one | | |
| 45 | 2-(1-methylindol-5-yl)- | 274.32 | |
| | 1H-quinolin-4-one | 275.2 | |
| 46 | 2-(2,3-difluorophenyl)- | 257.23258 | |
| | 1H-quinolin-4-one | .1 | |
| 47 | 2-(2,6-dimethylphenyl)- | 249.31250 | |
| | 1H-quinolin-4-one | .2 | |
| 48 | 2-(4-isopropoxy-3,5- | 307.39 | |
| | dimethyl-phenyl)-1H- | 308.2 | |
| | quinolin-4-one | | |
| 49 | 2-[4- | 305.25 | |
| | (trifluoromethoxy)phenyl]- | 306.1 | |
| | 1H-quinolin-4-one | | |
| 50 | 2-(2-methoxy-4-methyl- | 265.31 | |
| | phenyl)-1H-quinolin-4- | 266.2 | |
| | one | | |
| 51 | 2-(3,4-dimethylphenyl)- | 249.31 | |
| | 1H-quinolin-4-one | 250.2 | |
| 52 | 2-(2,5-dimethylphenyl)- | 249.31250 | |
| | 1H-quinolin-4-one | .2 | |
| 53 | 2-(4-methoxy-2-methyl- | 265.31 | |
| | phenyl)-1H-quinolin-4- | 266.2 | |
| | one | | |
| 54 | 2-[2-methyl-4- | 319.28 | |
| | (trifluoromethoxy)phenyl]- | 320.1 | |
| | 1H-quinolin-4-one | | |
| 55 | 2-(4-tert-butyl-2,6- | 305.41 | |
| | dimethyl-phenyl)-1H- | 306.59 | |
| | quinolin-4-one | | |
| 56 | 2-tetralin-6-yl-1H- | 275.34 | |
| | quinolin-4-one | 276.2 | |
| 57 | 2-[5-(trifluoromethyl)-3- | 290.24 | ¹H NMR (500 MHz, DMSO-d₆) δ (ppm) |
| | pyridyl]-1H-quinolin-4- | 291.2 | 9.44 (d, J = 2.2 Hz, 1H), 9.25 (d, J = 2.2 Hz, |
| | one | | 1H), 8.89 (d, J = 2.3 Hz, 1H), 8.38-8.30 |
| | | | (m, 2H), 8.02 (ddd, J = 8.6, 6.9, 1.5 Hz, 1H), |
| | | | 7.73 (t, J = 7.7 Hz, 1H), 7.43 (s, 1H). |
| 58 | 2-[5-methyl-6- | 304.27 | |
| | (trifluoromethyl)-3- | 305.27 | |
| | pyridyl]-1H-quinolin-4- | | |
| | one | | |
| 59 | 2-[6-methyl-5- | 304.27 | |
| | (trifluoromethyl)-3- | 305.32 | |
| | pyridyl]-1H-quinolin-4- | | |
| | one | | |
| 60 | 2-[2-methoxy-5- | 320.27321 | ¹H NMR (500 MHz, DMSO-d₆) δ (ppm) |
| | (trifluoromethyl)-3- | .2 | 8.82 (d, J = 2.5 Hz, 1H), 8.44 (d, J = 2.5 Hz, |
| | pyridyl]-1H-quinolin-4- | | 1H), 8.20 (d, J = 8.1 Hz, 1H), 7.81 (d, J = |
| | one | | 4.0 Hz, 2H), 7.55-7.45 (m, 1H), 6.64 (s, 1H), |
| | | | 4.04 (s, 3H). |
| 61 | 2-[2-ethoxy-5- | 334.29 | |
| | (trifluoromethyl)-3- | 335.4 | |
| | pyridyl]-1H-quinolin-4- | | |
| | one | | |
| 62 | 2-[2-isopropoxy-5- | 348.32 | ¹H NMR (500 MHz, DMSO-d₆) δ (ppm) |
| | (trifluoromethyl)-3- | 349.4 | 11.86 (s, 1H), 8.75 (d, J = 2.6 Hz, 1H), 8.36 |
| | pyridyl]-1H-quinolin-4- | | (d, J = 2.6 Hz, 1H), 8.13 (d, J = 8.1 Hz, 1H), |
| | one | | 7.81-7.59 (m, 2H), 7.38 (t, J = 7.5 Hz, 1H), |
| | | | 6.44-6.15 (m, 1H), 5.43 (hept, J = 6.3 Hz, |
| | | | 1H), 1.33 (d, J = 6.2 Hz, 6H). |
| 63 | 2-[4-fluoro-3- | 307.24 | |
| | (trifluoromethyl)phenyl]- | 308.1 | |
| | 1H-quinolin-4-one | | |
| 64 | 2-(2,3-dimethylphenyl)- | 249.31250 | |
| | 1H-quinolin-4-one | .2 | |
| 65 | 2-(o-tolyl)-1H-quinolin-4- | 235.28236 | |
| | one | .1 | |
| 66 | 2-(2-methoxyphenyl)-1H- | 251.28 | ¹H NMR (400 MHz, DMSO-d₆) δ (ppm) |
| | quinolin-4-one | 252.1 | 12.78 (s, 1H), 8.28-8.16 (m, 1H), 7.91- |
| | | | 7.77 (m, 2H), 7.64-7.48 (m, 3H), 7.27 (d, |
| | | | 1H), 7.16 (t, 1H), 6.59 (s, 1H), 3.86 (s, 3H). |
| 67 | 2-(5-methoxy-2-methyl- | 265.31 | |
| | phenyl)-1H-quinolin-4- | 266.2 | |
| | one | | |
| 68 | 2-(4-chloro-2-fluoro-3- | 287.72 | |
| | methyl-phenyl)-1H- | 288.1 | |
| | quinolin-4-one | | |
| 69 | 2-[2,3-difluoro-5- | 325.23326 | |
| | (trifluoromethyl)phenyl]- | .1 | |
| | 1H-quinolin-4-one | | |
| 70 | 2-(6-chloroindan-5-yl)- | 295.76 | |
| | 1H-quinolin-4-one | 296.1 | |
| 71 | 2-(2,5-dimethoxy-4- | 295.33 | |
| | methyl-phenyl)-1H- | 296.2 | |
| | quinolin-4-one | | |
| 72 | 2-(2-methoxy-4,5- | 279.33 | |
| | dimethyl-phenyl)-1H- | 280.2 | |
| | quinolin-4-one | | |
| 73 | 2-(2-fluoro-4,5-dimethyl- | 267.3 | |
| | phenyl)-1H-quinolin-4- | 268.2 | |
| | one | | |
| 74 | 2-(3-chloro-2,4-dimethyl- | 283.75 | |
| | phenyl)-1H-quinolin-4- | 284.2 | |
| | one | | |
| 75 | 2-(4-chloro-2-methyl- | 269.73 | |
| | phenyl)-1H-quinolin-4- | 270.1 | |
| | one | | |
| 76 | 2-(3-tert-butoxyphenyl)- | 293.36 | |
| | 1H-quinolin-4-one | 294.2 | |
| 77 | 2-[2-propoxy-5- | 347.33 | |
| | (trifluoromethyl)phenyl]- | 348.2 | |
| | 1H-quinolin-4-one | | |
| 78 | 2-[2-isopropoxy-5- | 347.33 | |
| | (trifluoromethyl)phenyl]- | 348.2 | |
| | 1H-quinolin-4-one | | |
| 79 | 2-(2-methoxy-4,6- | 279.33 | |
| | dimethyl-phenyl)-1H- | 280.2 | |
| | quinolin-4-one | | |
| 80 | 2-(4-tert-butyl-2-ethyl- | 305.41 | |
| | phenyl)-1H-quinolin-4- | 306 | |
| | one | | |
| 81 | 2-(4-chloro-3,5-dimethyl- | 283.75 | |
| | phenyl)-1H-quinolin-4- | 283 | |
| | one | | |
| 82 | 2-(3,5-dimethylphenyl)- | 249.31 | |
| | 1H-quinolin-4-one | 250.2 | |
| 83 | 2-(2-hydroxyphenyl)-1H- | 237.25238 | |
| | quinolin-4-one | .1 | |
| 84 | 2-[2-hydroxy-5- | 305.25 | |
| | (trifluoromethyl)phenyl]- | 306.1 | |
| | 1H-quinolin-4-one | | |
| 85 | 2-[5-chloro-2-methoxy-4- | 353.72 | ¹H NMR (400 MHz, DMSO-d₆) δ (ppm) |
| | (trifluoromethyl)phenyl]- | 354.3 | 11.83 (s, 1H), 8.11 (dd, J = 8.0, 1.5 Hz, 1H), |
| | 1H-quinolin-4-one | | 7.90 (s, 1H), 7.71-7.65 (m, 1H), 7.63-7.58 |
| | | | (m, 1H), 7.57 (s, 1H), 7.35 (t, J = 7.5 Hz, |
| | | | 1H), 6.16 (s, 1H), 3.94 (s, 3H). |
| 86 | 2-(2-isopropoxy-4,5- | 307.39 | ¹H NMR (400 MHz, DMSO-d₆) δ (ppm) |
| | dimethyl-phenyl)-1H- | 308.5 | 12.47 (s, 1H), 8.17 (dd, J = 8.0, 1.4 Hz, 1H), |
| | quinolin-4-one | | 7.84-7.73 (m, 2H), 7.47 (ddd, J = 8.1, 6.4, |
| | | | 1.6 Hz, 1H), 7.34 (s, 1H), 7.08 (s, 1H), 6.54 |
| | | | (s, 1H), 4.60 (hept, J = 6.1 Hz, 1H), 2.31 (s, |
| | | | 3H), 2.24 (s, 3H), 1.22 (d, J = 6.0 Hz, 6H). |

### Example 4

Method B: One Step General Procedure for Suzuki Coupling on Benzyl-Protected Intermediate A

A mixture of Intermediate A (1 eq), Intermediate B (1-2 eq, custom or commercial boronic acid or boronic ester), Palladium source (e.g. XPhos Pd G3, SPhos Pd G3, PdCl₂(dppf), with or without additional ligand (1-10 mol %, e.g X-Phos), base (2-3 eq, e.g. potassium carbonate or phosphate) in organic solvent (e.g. dioxane, DMSO, toluene) and water is degassed with nitrogen bubbling, sealed and heated at 60-120° C. overnight or subjected to microwave irradiation at 100-120° C. for 30-60 minutes. The reaction mixture is filtered, concentrated, and purified via silica gel column chromatography or reverse phase column chromatography to provide the desired product of formula (I), (II), or (III).

Method C: General Procedure for Suzuki Coupling of Benzyl-Protected Intermediate A with Intermediate B

Step 1: A mixture of Intermediate A (1 eq), Intermediate B (1-2 eq, custom or commercial boronic acid or boronic ester), Palladium source (1-5 mol %, e.g. PdCl₂(dppf) or PdCl₂(dtbpf), base (2-3 eq, eg. potassium phosphate) in organic solvent (e.g. dioxane, DMSO, toluene) and water is degassed with nitrogen bubbling nitrogen and stirred under nitrogen atmosphere at a temperature ranging from room temperature to 120° C. The reaction mixture is filtered and purified via silica gel column chromatography or reverse phase HPLC (C₁₈) to obtain a protected intermediate of a compound of formula (I), (II), or (III).

Step 2: A mixture of the protected intermediate and Pd/C is stirred in the appropriate solvent (e.g. methanol, ethanol, or ethyl acetate) under an atmosphere of hydrogen. The reaction mixture is filtered, concentrated, and purified via silica gel column chromatography or reverse phase column chromatography (C₁₈) to provide the desired product of formula (I), (II), or (III).

Alternatively, a solution of the protected intermediate in the appropriate solvent (DCM, dioxane or toluene) is treated with acid (e.g. HCl or TFA) and stirred at room temperature or 60-70° C. The mixture is neutralized and purified via silica gel column chromatography or reverse phase column chromatography to provide the desired product of formula (I), (II), or (III).

The following compounds (Table 3) were synthesized using the general Method B or C using custom or commercially available boronic acid or boronic esters.

TABLE 3

| | | LC/MS | |
|---|---|---|---|
| Cmpd. | | (m/z | |
| No. | Compound Name | calc.) | NMR (shifts in ppm) |
| 87 | 2-(4-tert-butyl-2-methyl- | 309.38 | ¹H NMR (400 MHz, DMSO-d₆) δ (ppm) |
| | phenyl)-6-fluoro-1H- | 310.22 | 11.90 (s, 1H), 7.75 (dd, J = 9.3, 3.0 Hz, 1H), |
| | quinolin-4-one | | 7.66 (dd, J = 9.1, 4.7 Hz, 1H), 7.58 (ddd, J = |
| | | | 9.1, 8.2, 3.0 Hz, 1H), 7.43 (d, J = 1.8 Hz, |
| | | | 1H), 7.41-7.33 (m, 2H), 5.99 (d, J = 1.7 |
| | | | Hz, 1H), 2.31 (s, 3H), 1.33 (s, 9H). |
| 88 | 2-(4-tert-butyl-2-methyl- | 309.38 | |
| | phenyl)-7-fluoro-1H- | 310.02 | |
| | quinolin-4-one | | |
| 89 | 2-(4-tert-butyl-2-methoxy- | 325.38 | ¹H NMR (400 MHz, DMSO-d₆) δ (ppm) |
| | phenyl)-6-fluoro-1H- | 326.2 | 11.93 (s, 1H), 7.72 (ddd, J = 19.6, 9.2, 3.8 |
| | quinolin-4-one | | Hz, 2H), 7.58 (ddd, J = 9.1, 8.2, 3.0 Hz, |
| | | | 1H), 7.42 (d, J = 8.0 Hz, 1H), 7.18 (d, J = |
| | | | 1.7 Hz, 1H), 7.14 (dd, J = 8.0, 1.7 Hz, 1H), |
| | | | 6.14 (s, 1H), 3.86 (s, 3H), 1.35 (s, 9H). |
| 90 | 6-fluoro-2-[2-methyl-4-[1- | 361.33 | ¹H NMR (400 MHz, DMSO-d₆) δ (ppm) |
| | (trifluoromethyl) | 362.37 | 11.96 (s, 1H), 7.76 (dd, J = 9.4, 2.9 Hz, 1H), |
| | cyclopropyl]phenyl]-1H-quinolin- | | 7.66 (dd, J = 9.1, 4.7 Hz, 1H), 7.59 (ddd, J = |
| | 4-one | | 9.1, 8.2, 2.9 Hz, 1H), 7.51 (s, 1H), 7.46 (d, J = |
| | | | 1.1 Hz, 2H), 6.00 (s, 1H), 2.31 (s, 3H), |
| | | | 1.44-1.35 (m, 2H), 1.23-1.11 (m, 2H). ¹⁹F |
| | | | NMR (376 MHz, DMSO-d₆) δ (ppm)- |
| | | | 68.16, −117.62-−117.96 (m). |
| 91 | 2-(4-ethyl-3,5-dimethyl- | 295.35 | |
| | phenyl)-6-fluoro-1H- | 296.2 | |
| | quinolin-4-one | | |
| 92 | 2-(4-tert-butyl-2-methyl- | 292.37 | |
| | phenyl)-1H-1,6- | 293.2 | |
| | naphthyridin-4-one | | |
| 93 | 2-[2-methyl-4-[1- | 344.33 | ¹H NMR (400 MHz, DMSO-d₆) δ (ppm) |
| | (trifluoromethyl) | 345.43 | 12.04 (s, 1H), 9.22 (s, 1H), 8.61 (d, J = 5.8 |
| | cyclopropyl]phenyl]-1H-1,6- | | Hz, 1H), 7.52 (s, 1H), 7.51-7.41 (m, 3H), |
| | naphthyridin-4-one | | 6.13 (s, 1H), 2.33 (s, 3H), 1.43-1.36 (m, |
| | | | 2H), 1.18 (s, 2H). |
| 94 | 2-[4-(3,3- | 343.34 | ¹H NMR (400 MHz, CDCl₃) δ (ppm) 7.92 |
| | difluorocyclobutyl)-2- | 344.25 | (dd, J = 8.9, 2.9 Hz, 1H), 7.86 (s, 1H), 7.39 |
| | methyl-phenyl]-6-fluoro- | | (ddd, J = 9.0, 7.6, 2.9 Hz, 1H), 7.20 (d, J = |
| | 1H-quinolin-4-one | | 7.9 Hz, 1H), 7.05 (s, 1H), 6.95 (s, 1H), 6.38 |
| | | | (s, 1H), 3.34-3.06 (m, 1H), 2.95 (tdd, J = |
| | | | 14.4, 8.9, 5.2 Hz, 2H), 2.55 (td, J = 23.1, |
| | | | 12.1 Hz, 2H), 2.33 (s, 3H). |
| 95 | 2-(4-tert-butyl-2-methyl- | 292.37 | |
| | phenyl)-1H-1,5- | 293.2 | |
| | naphthyridin-4-one | | |
| 96 | 2-(4-tert-butyl-2-ethyl- | 323.4 | ¹H NMR (400 MHz, DMSO-d₆) δ (ppm) |
| | phenyl)-6-fluoro-1H- | 324.5 | 11.95 (s, 1H), 7.76 (dd, J = 9.4, 3.0 Hz, 1H), |
| | quinolin-4-one | | 7.66 (dd, J = 9.1, 4.7 Hz, 1H), 7.58 (ddd, J = |
| | | | 9.2, 8.2, 3.0 Hz, 1H), 7.45 (d, J = 1.9 Hz, |
| | | | 1H), 7.39 (dd, J = 8.0, 2.0 |
| | | | Hz, 1H), 7.33 (d, J = 8.0 Hz, 1H), 5.99 (s, |
| | | | 1H), 2.64 (q, J = 7.6 Hz, 2H), 1.33 (s, 9H), |
| | | | 1.09 (t, J = 7.5 Hz, 3H). |
| 97 | 2-(4-tert-butyl-2-ethyl- | 306.4 | |
| | phenyl)-1H-1,6- | 307.3 | |
| | naphthyridin-4-one | | |
| 98 | 2-(4-tert-butyl-2-methoxy- | 308.37 | |
| | phenyl)-1H-1,6- | 309.4 | |
| | naphthyridin-4-one | | |
| 99 | 2-(4-tert-butyl-2-methyl- | 322.4 | |
| | phenyl)-6-methoxy-1H- | 323.3 | |
| | 1,5-naphthyridin-4-one | | |
| 100 | 2-[4-(1,1-dimethylpropyl)- | 306.4 | |
| | 2-methyl-phenyl]-1H-1,6- | 307.2 | |
| | naphthyridin-4-one | | |
| 101 | 2-[2-methyl-4-[1- | 344.33 | |
| | (trifluoromethyl)cyclopropyl] | 345.2 | |
| | phenyl]-1H-1,5- | | |
| | naphthyridin-4-one | | |
| 102 | 2-(6-tert-butyl-2-methyl- | 293.36 | ¹H NMR (400 MHz, DMSO-d₆) δ (ppm) |
| | 3-pyridyl)-1H-1,6- | 294.3 | 13.22 (s, 1H), 9.41 (s, 1H), 8.75 (d, J = 7.7 |
| | naphthyridin-4-one | | Hz, 1H), 8.00 (d, J = 6.7 Hz, 1H), 7.93 (d, J = |
| | | | 8.1 Hz, 1H), 7.54 (d, J = 8.1 Hz, 1H), 6.49 |
| | | | (s, 1H), 2.59 (s, 3H), 1.38 (s, 9H). |
| 103 | 2-(4-tert-butyl-2-methyl- | 308.37 | |
| | phenyl)-5-oxido-1H-1,5- | 309.2 | |
| | naphthyridin-5-ium-4-one | | |
| 104 | 2-(4-chloro-2-methoxy-6- | 299.75 | ¹H NMR (400 MHz, DMSO-d₆) δ (ppm) |
| | methyl-phenyl)-1H- | 300.3 | 11.82 (s, 1H), 8.11 (dd, J = 8.1, 1.5 Hz, 1H), |
| | quinolin-4-one | | 7.65 (ddd, J = 8.4, 6.9, 1.6 Hz, 1H), 7.54 (d, |
| | | | J = 8.2 Hz, 1H), 7.37-7.32 (m, 1H), 7.15- |
| | | | 7.08 (m, 2H), 5.91 (s, 1H), 3.76 (s, 3H), 2.16 |
| | | | (s, 3H). |
| 105 | 2-(4-tert-butyl-2-methyl- | 316.4 | ¹H NMR (400 MHz, DMSO-d₆) δ (ppm) |
| | phenyl)-4-oxo-1H- | 317.64 | 12.17 (s, 1H), 7.90 (dd, J = 8.0, 1.5 Hz, 1H), |
| | quinoline-5-carbonitrile | | 7.86-7.73 (m, 2H), 7.44 (d, J = 1.7 Hz, |
| | | | 1H), 7.39 (t, J = 1.5 Hz, 2H), 6.11 (s, 1H), |
| | | | 2.32 (s, 3H), 1.33 (s, 9H). |
| 106 | methyl 2-(4-tert-butyl-2- | 349.42 | ¹H NMR (400 MHz, CD₃OD) δ (ppm) 8.98 |
| | methyl-phenyl)-4-oxo-1H- | 350.38 | (d, J = 1.9 Hz, 1H), 8.27 (dd, J = 8.8, 2.0 |
| | quinoline-6-carboxylate | | Hz, 1H), 7.66 (d, J = 8.6 Hz, 1H), 7.47- |
| | | | 7.33 (m, 3H), 6.31 (s, 1H), 3.97 (s, 3H), 2.36 |
| | | | (s, 3H), 1.36 (s, 9H). |
| 107 | 2-(4-tert-butyl-2-methyl- | 308.37 | |
| | phenyl)-6-oxido-1H-1,6- | 309.2 | |
| | naphthyridin-6-ium-4-one | | |
| 108 | 2-(6-tert-butyl-2-methyl- | 310.37 | |
| | 3-pyridyl)-6-fluoro-1H- | 311.4 | |
| | quinolin-4-one | | |
| 109 | 2-(4-tert-butyl-2-methoxy- | 332.4 | |
| | phenyl)-4-oxo-1H- | 333.5 | |
| | quinoline-5-carbonitrile | | |
| 110 | 2-(4-tert-butyl-2-chloro- | 312.79 | |
| | phenyl)-1H-1,6- | 313.2 | |
| | naphthyridin-4-one | | |
| 111 | 2-(4-tert-butyl-2-methyl- | 292.37 | |
| | phenyl)-1H-1,8- | 293.2 | |
| | naphthyridin-4-one | | |
| 112 | ethyl 2-(4-tert-butyl-2- | 381.44 | ¹H NMR (400 MHz, CD₃OD) δ (ppm) 7.94 |
| | methyl-phenyl)-6-fluoro- | 382.5 | (dd, J = 9.1, 2.9 Hz, 1H), 7.62 (dd, J = 9.1, |
| | 4-oxo-1H-quinoline-3- | | 4.5 Hz, 1H), 7.55 (ddd, J = 9.1, 7.9, 2.9 Hz, |
| | carboxylate | | 1H), 7.41 (d, J = 2.0 Hz, 1H), 7.36 (dd, J = |
| | | | 8.0, 2.0 Hz, 1H), 7.24 (d, J = 8.0 Hz, 1H), |
| | | | 4.02-3.87 (m, 2H), 2.28 (s, 3H), 1.35 (s, |
| | | | 9H), 0.81 (t, J = 7.1 Hz, 3H). |
| 113 | 2-(4-tert-butyl-2,5- | 305.41 | ¹H NMR (400 MHz, DMSO-d₆) δ (ppm) |
| | dimethyl-phenyl)-1H- | 306.3 | 12.82 (s, 1H), 8.21 (dd, J = 7.9, 1.2 Hz, 1H), |
| | quinolin-4-one | | 7.84-7.76 (m, 2H), 7.54-7.48 (m, 1H), |
| | | | 7.36 (s, 1H), 7.26 (s, 1H), 6.45 (s, 1H), 2.54 |
| | | | (s, 3H), 2.29 (s, 3H), 1.42 (s, 9H). |
| 114 | 2-(4-tert-butyl-2,5- | 306.4 | ¹H NMR (400 MHz, CD₃OD) δ (ppm) 9.47 |
| | dimethyl-phenyl)-1H-1,6- | 307.3 | (s, 1H), 8.72 (dd, J = 6.9, 1.1 Hz, 1H), 7.97 |
| | naphthyridin-4-one | | (d, J = 6.9 Hz, 1H), 7.41 (s, 1H), 7.25 (s, |
| | | | 1H), 6.48 (s, 1H), 2.59 (s, 3H), 2.36 (s, 3H), |
| | | | 1.45 (s, 9H). |
| 115 | 2-(2-methyl-4- | 307.46 | ¹H NMR (400 MHz, DMSO-d₆) δ (ppm) |
| | trimethylsilyl-phenyl)-1H- | 308.2 | 12.79 (s, 1H), 8.24-8.16 (m, 1H), 7.83- |
| | quinolin-4-one | | 7.72 (m, 2H), 7.62-7.41 (m, 4H), 6.41 (s, |
| | | | 1H), 2.33 (s, 3H), 0.30 (s, 9H). |
| 116 | 2-(2-methyl-4- | 308.45 | |
| | trimethylsilyl-phenyl)-1H- | 309.2 | |
| | 1,6-naphthyridin-4-one | | |
| 117 | 2-(4,5-dichloro-2-methyl- | 304.17 | ¹H NMR (400 MHz, DMSO-d₆) δ (ppm) |
| | phenyl)-1H-quinolin-4- | 305.1 | 8.24 (d, J = 8.2 Hz, 1H), 7.90-7.84 (m, |
| | one | | 3H), 7.79 (s, 1H), 7.61-7.53 (m, 1H), 6.59 |
| | | | (s, 1H), 2.31 (s, 3H). |
| 118 | 2-(4,5-dichloro-2-methyl- | 305.16 | ¹H NMR (400 MHz, DMSO-d₆) δ (ppm) |
| | phenyl)-1H-1,6- | 306.07 | 13.09 (s, 1H), 9.38 (s, 1H), 8.73 (d, J = 6.6 |
| | naphthyridin-4-one | | Hz, 1H), 7.95 (d, J = 6.6 Hz, 1H), 7.80 (d, |
| | | | J = 21.4 Hz, 2H), 6.41 (s, 1H), 2.34 (s, 3H). |
| 119 | 6-fluoro-2-[2-methyl-4- | 363.35 | |
| | (2,2,2-trifluoro-1,1- | 364.2 | |
| | dimethyl-ethyl)phenyl]- | | |
| | 1H-quinolin-4-one | | |
| 120 | 7-fluoro-2-[2-methyl-4- | 363.35 | |
| | (2,2,2-trifluoro-1,1- | 364.2 | |
| | dimethyl-ethyl)phenyl]- | | |
| | 1H-quinolin-4-one | | |
| 121 | 6-fluoro-2-[2-methyl-4-(1- | 321.39 | ¹H NMR (400 MHz, DMSO-d₆) δ (ppm) |
| | methylcyclobutyl)phenyl]- | 322.4 | 11.90 (s, 1H), 7.75 (dd, J = 9.4, 3.0 Hz, 1H), |
| | 1H-quinolin-4-one | | 7.66 (dd, J = 9.1, 4.7 Hz, 1H), 7.58 (td, J = |
| | | | 8.6, 3.0 Hz, 1H), 7.37 (d, J = 7.8 Hz, 1H), |
| | | | 7.19 (d, J = 1.9 Hz, 1H), 7.17-7.11 (m, |
| | | | 1H), 5.99 (s, 1H), 2.39-2.32 (m, 2H), 2.30 |
| | | | (s, 3H), 2.16-2.01 (m, 3H), 1.85-1.73 (m, |
| | | | 1H), 1.45 (s, 3H). |
| 122 | 7-fluoro-2-[2-methyl-4-(1- | 321.39 | ¹H NMR (400 MHz, DMSO-d₆) δ (ppm) |
| | methylcyclobutyl)phenyl]- | 322.4 | 11.80 (s, 1H), 8.16 (dd, J = 8.9, 6.4 Hz, 1H), |
| | 1H-quinolin-4-one | | 7.36 (d, J = 7.9 Hz, 1H), 7.29 (dd, J = 10.1, |
| | | | 2.5 Hz, 1H), 7.23-7.10 (m, 3H), 5.98 (s, |
| | | | 1H), 2.38-2.31 (m, 2H), 2.30 (s, 3H), 2.18- |
| | | | 2.01 (m, 3H), 1.85-1.73 (m, 1H), 1.45 (s, |
| | | | 3H). |
| 123 | 4-oxo-2-(2,4,5- | 288.34 | ¹H NMR (400 MHz, DMSO-d₆) δ (ppm) |
| | trimethylphenyl)-1H- | 289.4 | 12.09 (s, 1H), 7.91 (dd, J = 8.0, 1.6 Hz, 1H), |
| | quinoline-5-carbonitrile | | 7.84-7.76 (m, 2H), 7.23 (s, 1H), 7.18 (s, |
| | | | 1H), 6.06 (s, 1H), 2.27-2.24 (m, 9H). |
| 124 | 2-(2,4,5-trimethylphenyl)- | 263.33 | |
| | 1H-quinolin-4-one | 264.5 | |
| 125 | 2-(4-tert-butyl-2-methyl- | 309.38 | |
| | phenyl)-5-fluoro-1H- | 310.3 | |
| | quinolin-4-one | | |
| 126 | 2-(4-tert-butyl-2-methyl- | 305.41 | ¹H NMR (400 MHz, DMSO-d₆) δ (ppm) |
| | phenyl)-6-methyl-1H- | 306.5 | 11.75 (s, 1H), 7.91 (s, 1H), 7.53-7.46 (m, |
| | quinolin-4-one | | 2H), 7.42 (s, 1H), 7.40-7.32 (m, 2H), 5.96 |
| | | | (s, 1H), 2.42 (s, 3H), 2.30 (s, 3H), 1.33 (s, |
| | | | 9H) |
| 127 | 2-(4-tert-butyl-2-methyl- | 305.41 | ¹H NMR (400 MHz, DMSO-d₆) δ (ppm) |
| | phenyl)-3-methyl-1H- | 306.5 | 11.58 (s, 1H), 8.12 (dd, J = 8.1, 1.6 Hz, 1H), |
| | quinolin-4-one | | 7.59 (ddd, J = 8.3, 6.7, 1.5 Hz, 1H), 7.53 |
| | | | (dd, J = 8.4, 1.3 Hz, 1H), 7.45 (d, J = 1.9 |
| | | | Hz, 1H), 7.39 (dd, J = 8.0, 2.0 Hz, 1H), 7.29 |
| | | | (ddd, J = 8.1, 6.7, 1.3 Hz, 1H), 7.27 (d, J = |
| | | | 8.0 Hz, 1H), 2.16 (s, 3H), 1.72 (s, 3H), 1.34 |
| | | | (s, 9H) |
| 128 | 2-(4-tert-butyl-2-methyl- | 323.4 | ¹H NMR (400 MHz, DMSO-d₆) δ (ppm) |
| | phenyl)-6-fluoro-3- | 324.5 | 11.75 (s, 1H), 7.76 (dd, J = 9.5, 3.0 Hz, 1H), |
| | methyl-1H-quinolin-4-one | | 7.61 (dd, J = 9.1, 4.7 Hz, 1H), 7.53 (ddd, J = |
| | | | 9.1, 8.3, 3.0 Hz, 1H), 7.45 (d, J = 2.0 Hz, |
| | | | 1H), 7.39 (dd, J = 7.9, 2.0 Hz, 1H), 7.27 (d, |
| | | | J = 8.0 Hz, 1H), 2.15 (s, 3H), 1.72 (s, 3H), |
| | | | 1.34 (s, 9H) |
| 129 | 2-(4-tert-butyl-2,5- | 330.42 | ¹H NMR (400 MHz, DMSO-d₆) δ (ppm) |
| | dimethyl-phenyl)-4-oxo- | 331.3 | 12.20 (s, 1H), 7.91 (dd, J = 8.1, 1.6 Hz, 1H), |
| | 1H-quinoline-5- | | 7.86-7.76 (m, 2H), 7.33 (s, 1H), 7.22 (s, |
| | carbonitrile | | 1H), 6.13 (s, 1H), 2.53 (s, 3H), 2.28 (s, 3H), |
| | | | 1.41 (s, 9H). |
| 130 | 2-(4-tert-butyl-2-methyl- | 359.38 | |
| | phenyl)-6- | 360.5 | |
| | (trifluoromethyl)-1H- | | |
| | quinolin-4-one | | |
| 131 | 2-[2-methyl-4-(2,2,2- | 370.37 | ¹H NMR (400 MHz, DMSO-d₆) δ (ppm) |
| | trifluoro-1, 1-dimethyl- | 371.4 | 12.18 (s, 1H), 7.91-7.77 (m, 3H), 7.61- |
| | ethyl)phenyl]-4-oxo-1H- | | 7.46 (m, 3H), 6.12 (d, J = 1.7 Hz, 1H), 2.35 |
| | quinoline-5-carbonitrile | | (s, 3H), 1.61 (s, 6H). |
| 132 | 2-(6-tert-butyl-2-methyl- | 317.38 | ¹H NMR (400 MHz, DMSO-d₆) δ (ppm) |
| | 3-pyridyl)-4-oxo-1H- | 318.4 | 12.23 (s, 1H), 7.91-7.78 (m, 4H), 7.47 (d, |
| | quinoline-5-carbonitrile | | J = 8.0 Hz, 1H), 6.20 (s, 1H), 2.51 (s, 3H), |
| | | | 1.36 (s, 9H). |
| 133 | 2-(4-tert-butyl-2-methyl- | 323.4 | ¹H NMR (400 MHz, DMSO-d₆) δ (ppm) |
| | phenyl)-7-fluoro-3- | 324.4 | 11.63 (s, 1H), 8.17 (dd, J = 9.0, 6.4 Hz, 1H), |
| | methyl-1H-quinolin-4-one | | 7.45 (d, J = 2.0 Hz, 1H), 7.39 (dd, J = 8.0, |
| | | | 2.0 Hz, 1H), 7.27 (d, J = 8.0 Hz, 1H), 7.23 |
| | | | (dd, J = 10.2, 2.5 Hz, 1H), 7.15 (td, J = 8.8, |
| | | | 2.5 Hz, 1H), 2.16 (s, 3H), 1.71 (s, 3H), 1.34 |
| | | | (s, 9H) |
| 134 | 2-(4-tert-butylphenyl)-3- | 291.39 | ¹H NMR (400 MHz, DMSO-d₆) δ (ppm) |
| | methyl-1H-quinolin-4-one | 292.5 | 11.57 (s, 1H), 8.12 (d, J = 8.1 Hz, 1H), 7.64- |
| | | | 7.57 (m, 4H), 7.52-7.46 (m, 2H), 7.29 |
| | | | (ddd, J = 8.1, 5.0, 3.1 Hz, 1H), 1.90 (s, 3H), |
| | | | 1.36 (s, 9H) |
| 135 | 2-(4-tert-butyl-2-methyl- | 321.41 | ¹H NMR (400 MHz, DMSO-d₆) δ (ppm) |
| | phenyl)-5-methoxy-1H- | 322.6 | 13.12 (s, 1H), 7.76 (td, J = 8.3, 7.7, 4.5 Hz, |
| | quinolin-4-one | | 1H), 7.47 (s, 1H), 7.43 (s, 2H), 7.38 (dd, J = |
| | | | 8.5, 4.9 Hz, 1H), 7.05 (dd, J = 8.3, 4.6 Hz, |
| | | | 1H), 6.55 (d, J = 15.0 Hz, 1H), 3.94 (d, J = |
| | | | 1.8 Hz, 3H), 2.32 (s, 3H), 1.33 (s, 9H). |
| 136 | 2-(4-tert-butyl-2,5- | 323.4 | ¹H NMR (400 MHz, DMSO-d₆) δ (ppm) |
| | dimethyl-phenyl)-5- | 324.3 | 12.06 (s, 1H), 7.68-7.59 (m, 1H), 7.44 (d, J = |
| | fluoro-1H-quinolin-4-one | | 8.4 Hz, 1H), 7.33 (s, 1H), 7.20 (s, 1H), |
| | | | 7.05 (dd, J = 11.9, 7.9 Hz, 1H), 6.04 (s, 1H), |
| | | | 2.53 (s, 3H), 2.27 (s, 3H), 1.41 (s, 9H). |
| 137 | 2-(4-tert-butyl-2-methyl- | 321.41 | ¹H NMR (400 MHz, DMSO-d₆) δ (ppm) |
| | phenyl)-6-methoxy-1H- | 322.6 | 11.75 (s, 1H), 7.55 (d, J = 9.0 Hz, 1H), 7.52 |
| | quinolin-4-one | | (d, J = 3.0 Hz, 1H), 7.42 (d, J = 1.9 Hz, 1H), |
| | | | 7.38 (dd, J = 8.1, 1.9 Hz, 1H), 7.34 (d, J = |
| | | | 8.0 Hz, 1H), 7.30 (dd, J = 9.0, 3.0 Hz, 1H), |
| | | | 5.95 (s, 1H), 3.85 (s, 3H), 2.30 (s, 3H), 1.33 |
| | | | (s, 9H) |
| 138 | 1-[3-methyl-4-(4-oxo-1H- | 300.35 | ¹H NMR (400 MHz, DMSO-d₆) δ (ppm) |
| | quinolin-2- | 301.4 | 13.62 (s, 1H), 8.30-8.22 (m, 1H), 7.97- |
| | yl)phenyl] | | 7.83 (m, 2H), 7.66-7.51 (m, 2H), 7.43- |
| | cyclopropanecarbonitrile | | 7.35 (m, 2H), 6.75 (s, 1H), 2.35 (s, 3H), 1.88- |
| | | | 1.79 (m, 2H), 1.61 (q, J = 5.0 Hz, 2H). |
| 139 | 2-(4-tert-butyl-2-methyl- | 339.4 | ¹H NMR (400 MHz, DMSO-d₆) δ (ppm) |
| | phenyl)-6-fluoro-5- | 340.6 | 11.64 (s, 1H), 7.59 (dd, J = 10.3, 9.2 Hz, |
| | methoxy-1H-quinolin-4- | | 1H), 7.43-7.31 (m, 4H), 5.86 (s, 1H), 3.85 |
| | one | | (s, 3H), 2.31 (s, 3H), 1.32 (s, 9H). |
| 140 | 2-(4-tert-butyl-2,5- | 353.43 | ¹H NMR (400 MHz, DMSO-d₆) δ (ppm) |
| | dimethyl-phenyl)-6- | 354.7 | 11.62 (s, 1H), 7.59 (dd, J = 10.2, 9.2 Hz, |
| | fluoro-5-methoxy-1H- | | 1H), 7.35 (dd, J = 9.2, 4.4 Hz, 1H), 7.31 (s, |
| | quinolin-4-one | | 1H), 7.17 (s, 1H), 5.85 (d, J = 1.6 Hz, 1H), |
| | | | 3.85 (s, 3H), 2.52 (s, 3H), 2.26 (s, 3H), 1.40 |
| | | | (s, 9H). |
| 141 | 6-fluoro-5-methoxy-2-[2- | 393.37 | ¹H NMR (400 MHz, DMSO-d₆) δ (ppm) |
| | methyl-4-(2,2,2-trifluoro- | 394.6 | 11.70 (s, 1H), 7.67-7.55 (m, 2H), 7.53 (d, J = |
| | 1,1-dimethyl- | | 8.8 Hz, 1H), 7.45 (d, J = 8.1 Hz, 1H), 7.35 |
| | ethyl)phenyl]-1H- | | (dd, J = 9.2, 4.4 Hz, 1H), 5.88 (s, 1H), 3.86 |
| | quinolin-4-one | | (s, 3H), 2.34 (s, 3H), 1.60 (s, 6H). |
| 142 | 6-fluoro-2-[2-methyl-4-(1- | 307.36 | |
| | methylcyclopropyl)phenyl | 308.2 | |
| | 1-1H-quinolin-4-one | | |
| 143 | 2-[2-methyl-4-(1- | 314.38 | ¹H NMR (400 MHz, DMSO-d₆) δ (ppm) |
| | methylcyclopropyl)phenyl]- | 315.2 | 12.00 (s, 1H), 7.88 (d, 1H), 7.82-7.73 (m, |
| | 4-oxo-1H-quinoline-5- | | 2H), 7.34 (d, 1H), 7.26 (s, 1H), 7.22 (d, 1H), |
| | carbonitrile | | 6.06 (s, 1H), 2.30 (s, 3H), 1.42 (s, 3H), 0.93- |
| | | | 0.85 (m, 2H), 0.83-0.74 (m, 2H). |
| 144 | 2-(4-tert-butyl-2,5- | 339.86 | ¹H NMR (400 MHz, CD₃OD) δ (ppm) 7.93- |
| | dimethyl-phenyl)-5- | 340.3 | 7.87 (m, 2H), 7.78 (dd, J = 5.1, 3.8 Hz, 1H), |
| | chloro-1H-quinolin-4-one | | 7.46 (s, 1H), 7.31 (s, 1H), 6.98 (s, 1H), 2.61 |
| | | | (s, 3H), 2.36 (s, 3H), 1.47 (s, 9H). |
| 145 | 2-(4-tert-butyl-2-methyl- | 339.4 | ¹H NMR (400 MHz, DMSO-d₆) δ (ppm) |
| | phenyl)-7-fluoro-5- | 340.5 | 11.46 (s, 1H), 7.45-7.28 (m, 3H), 6.81 (dd, |
| | methoxy-1H-quinolin-4- | | J = 10.0, 2.4 Hz, 1H), 6.67 (dd, J = 11.9, 2.4 |
| | one | | Hz, 1H), 5.80 (s, 1H), 3.83 (s, 3H), 2.29 (s, |
| | | | 3H), 1.32 (s, 9H). |
| 146 | 2-(4-tert-butyl-2,5- | 353.43 | ¹H NMR (400 MHz, DMSO-d₆) δ (ppm) |
| | dimethyl-phenyl)-7- | 354.5 | 12.96 (s, 1H), 7.35 (s, 1H), 7.24 (s, 1H), |
| | fluoro-5-methoxy-1H- | | 7.09 (dd, J = 9.6, 2.5 Hz, 1H), 6.96 (dd, J = |
| | quinolin-4-one | | 11.6, 2.4 Hz, 1H), 6.49 (s, 1H), 3.93 (s, 3H), |
| | | | 2.53 (s, 3H), 2.28 (s, 3H), 1.41 (s, 9H). |
| 147 | 7-fluoro-5-methoxy-2-[2- | 393.37 | ¹H NMR (400 MHz, DMSO-d₆) δ (ppm) |
| | methyl-4-(2,2,2-trifluoro- | 394.3 | 11.53 (s, 1H), 7.57 (s, 1H), 7.52 (d, J = 8.9 |
| | 1,1-dimethyl- | | Hz, 1H), 7.43 (d, J = 8.1 Hz, 1H), 6.85- |
| | ethyl)phenyl]-1H- | | 6.76 (m, 1H), 6.72-6.60 (m, 1H), 5.83 (s, |
| | quinolin-4-one | | 1H), 3.84 (s, 3H), 2.33 (s, 3H), 1.60 (s, 6H). |
| 148 | 2-(4-tert-butyl-2-methyl- | 339.4 | ¹H NMR (400 MHz, DMSO-d₆) δ (ppm) |
| | phenyl)-5-fluoro-7- | 340.4 | 11.80 (s, 1H), 7.45-7.32 (m, 3H), 6.87 (d, |
| | methoxy-1H-quinolin-4- | | J = 2.4 Hz, 1H), 6.74 (dd, J = 13.5, 2.4 Hz, |
| | one | | 1H), 5.90 (s, 1H), 3.83 (s, 3H), 2.31 (s, 3H), |
| | | | 1.32 (s, 9H). |
| 149 | 2-(4-tert-butyl-2,5- | 353.43 | ¹H NMR (400 MHz, DMSO-d₆) δ (ppm) |
| | dimethyl-phenyl)-5- | 354.4 | 12.31 (s, 1H), 7.33 (s, 1H), 7.21 (s, 1H), |
| | fluoro-7-methoxy-1H- | | 6.97 (d, J = 2.4 Hz, 1H), 6.85 (dd, J = 13.4, |
| | quinolin-4-one | | 2.3 Hz, 1H), 6.13 (s, 1H), 3.86 (s, 3H), 2.53 |
| | | | (s, 3H), 2.28 (s, 3H), 1.41 (s, 9H). |
| 150 | 2-[2,5-dimethyl-4-[1- | 361.33 | ¹H NMR (400 MHz, DMSO-d₆) δ (ppm) |
| | (trifluoromethyl)vinyl] | 362.5 | 12.04 (s, 1H), 7.77 (dd, J = 9.3, 3.0 Hz, 1H), |
| | phenyl]-6-fluoro-1H-quinolin- | | 7.69 (dd, J = 9.1, 4.8 Hz, 1H), 7.61 (td, J = |
| | 4-one | | 8.6, 3.0 Hz, 1H), 7.43 (s, 1H), 7.23 (s, 1H), |
| | | | 6.32 (d, J = 1.7 Hz, 1H), 6.05 (s, 1H), 5.81 |
| | | | (d, J = 1.3 Hz, 1H), 2.28 (s, 6H). |
| 151 | 2-[2,5-dimethyl-4-[1- | 391.36 | |
| | (trifluoromethyl)vinyl] | 392.3 | |
| | phenyl]-6-fluoro-5-methoxy- | | |
| | 1H-quinolin-4-one | | |
| 152 | 2-(4-tert-butyl-2-methyl- | 309.38 | ¹H NMR (400 MHz, DMSO-d₆) δ (ppm) |
| | phenyl)-8-fluoro-1H- | 310.5 | 11.80 (s, 1H), 7.93 (d, J = 8.1 Hz, 1H), 7.56 |
| | quinolin-4-one | | (dd, J = 10.7, 8.4 Hz, 1H), 7.39-7.31 (m, |
| | | | 4H), 5.99 (s, 1H), 2.30 (s, 3H), 1.33 (s, 9H). |
| 153 | 2-(4-tert-butyl-2,5- | 323.4 | |
| | dimethyl-phenyl)-8- | 324.3 | |
| | fluoro-1H-quinolin-4-one | | |
| 154 | 2-(4-tert-butyl-2-methyl- | 327.37 | ¹H NMR (400 MHz, DMSO-d₆) δ (ppm) |
| | phenyl)-6,7-difluoro-1H- | 328.4 | 11.96 (s, 1H), 7.97 (dd, J = 11.0, 8.8 Hz, |
| | quinolin-4-one | | 1H), 7.53 (dd, J = 11.2, 6.8 Hz, 1H), 7.43 (s, |
| | | | 1H), 7.41-7.33 (m, 2H), 6.03 (s, 1H), 2.31 |
| | | | (s, 3H), 1.32 (s, 9H) |
| 155 | 2-(4-tert-butyl-2-methyl- | 334.39 | ¹H NMR (400 MHz, DMSO-d₆) δ (ppm) |
| | phenyl)-7-fluoro-4-oxo- | 335.5 | 12.15 (s, 1H), 7.87 (dd, J = 8.5, 2.5 Hz, 1H), |
| | 1H-quinoline-5- | | 7.59 (dd, J = 9.4, 2.5 Hz, 1H), 7.44 (s, 1H), |
| | carbonitrile | | 7.42-7.35 (m, 2H), 6.10 (s, 1H), 2.32 (s, |
| | | | 3H), 1.32 (s, 9H) |
| 156 | 2-(4-tert-butyl-2-methyl- | 357.39 | ¹H NMR (400 MHz, DMSO-d₆) δ (ppm) |
| | phenyl)-6,7-difluoro-5- | 358.3 | 12.06 (s, 1H), 7.43 (d, J = 1.9 Hz, 1H), 7.41- |
| | methoxy-1H-quinolin-4- | | 7.26 (m, 3H), 6.05 (s, 1H), 3.93 (s, 3H), |
| | one | | 2.31 (s, 3H), 1.32 (s, 9H). |
| 157 | 2-(4-tert-butyl-2-methyl- | 357.39 | ¹H NMR (400 MHz, DMSO-d₆) δ (ppm) |
| | phenyl)-5,6-difluoro-7- | 358.2 | 12.24 (s, 1H), 7.43 (d, J = 1.9 Hz, 1H), 7.41- |
| | methoxy-1H-quinolin-4- | | 7.34 (m, 2H), 7.14-7.09 (m, 1H), 6.08 (s, |
| | one | | 1H), 3.94 (s, 3H), 2.32 (s, 3H), 1.33 (s, 9H). |
| 158 | 2-(4-tert-butyl-2,5- | 371.42 | ¹H NMR (400 MHz, DMSO-d₆) δ (ppm) |
| | dimethyl-phenyl)-5,6- | 372.3 | 11.74 (s, 1H), 7.31 (s, 1H), 7.17 (s, 1H), |
| | difluoro-7-methoxy-1H- | | 7.06-7.01 (m, 1H), 5.87 (s, 1H), 3.92 (s, |
| | quinolin-4-one | | 3H), 2.52 (s, 3H), 2.26 (s, 3H), 1.40 (s, 9H). |
| 159 | 2-(4-tert-butyl-2-methyl- | 316.4 | ¹H NMR (400 MHz, DMSO-d₆) δ (ppm) |
| | phenyl)-4-oxo-1H- | 317.3 | 12.13 (s, 1H), 8.25 (d, J = 8.3 Hz, 1H), 8.00 |
| | quinoline-7-carbonitrile | | (s, 1H), 7.70 (dd, J = 8.3, 1.5 Hz, 1H), 7.44 |
| | | | (s, 1H), 7.43-7.35 (m, 2H), 6.13 (s, 1H), |
| | | | 2.32 (s, 3H), 1.33 (s, 9H). |
| 160 | 6-fluoro-2-(1,1,6- | 321.39 | ¹H NMR (400 MHz, CD₃OD) δ (ppm) 7.98 |
| | trimethylindan-5-yl)-1H- | 322.3 | (d, J = 8.9 Hz, 1H), 7.81 (s, 1H), 7.68 (t, J = |
| | quinolin-4-one | | 8.7 Hz, 1H), 7.27 (s, 1H), 7.19 (s, 1H), 6.56 |
| | | | (s, 1H), 2.94 (t, J = 7.2 Hz, 2H), 2.32 (s, |
| | | | 3H), 1.99 (t, J = 7.2 Hz, 2H), 1.30 (s, 6H). |
| 161 | 2-[2,5-dimethyl-4-(2,2,2- | 384.39 | |
| | trifluoro-1,1-dimethyl- | 385.5 | |
| | ethyl)phenyl]-4-oxo-1H- | | |
| | quinoline-7-carbonitrile | | |
| 162 | 2-[2,5-dimethyl-4-(2,2,2- | 360.37 | ¹H NMR (400 MHz, DMSO-d₆) δ (ppm) |
| | trifluoro-1,1-dimethyl- | 361.3 | 12.43 (s, 1H), 9.32 (s, 1H), 8.67 (d, J = 6.3 |
| | ethyl)phenyl]-1H-1,6- | | Hz, 1H), 7.72 (d, J = 6.3 Hz, 1H), 7.55 (s, |
| | naphthyridin-4-one | | 1H), 7.33 (s, 1H), 6.27 (s, 1H), 2.56 (s, 3H), |
| | | | 2.33 (s, 3H), 1.73 (s, 6H). |
| 163 | 2-[2,5-dimethyl-4-(2,2,2- | 377.38 | |
| | trifluoro-1,1-dimethyl- | 378.3 | |
| | ethyl)phenyl]-6-fluoro- | | |
| | 1H-quinolin-4-one | | |
| 164 | 2-[2,5-dimethyl-4-(2,2,2- | 390.4 | |
| | trifluoro-1,1-dimethyl- | 391.3 | |
| | ethyl)phenyl]-6-methoxy- | | |
| | 1H-1,5-naphthyridin-4- | | |
| | one | | |
| 165 | 2-(4-tert-butyl-2-hydroxy- | 311.35 | |
| | phenyl)-6-fluoro-1H- | 312.4 | |
| | quinolin-4-one | | |
| 166 | ethyl 3-methyl-4-(4-oxo- | 307.34 | ¹H NMR (400 MHz, DMSO-d₆) δ (ppm) |
| | 1H-quinolin-2-yl)benzoate | 308.1 | 11.87 (s, 1H), 8.13 (dd, J = 8.2, 1.5 Hz, 1H), |
| | | | 7.98 (d, J = 1.7 Hz, 1H), 7.92 (dd, J = 7.9, |
| | | | 1.7 Hz, 1H), 7.68 (ddd, J = 8.4, 6.8, 1.5 Hz, |
| | | | 1H), 7.63-7.57 (m, 2H), 7.36 (ddd, J = 8.1, |
| | | | 6.8, 1.2 Hz, 1H), 6.01 (s, 1H), 4.36 (q, J = |
| | | | 7.1 Hz, 2H), 2.38 (s, 3H), 1.35 (t, J = 7.1 |
| | | | Hz, 3H) |
| 167 | 2-[2-methyl-4-(2,2,2- | 346.35 | |
| | trifluoro-1,1-dimethyl- | 347.2 | |
| | ethyl)phenyl]-1H-1,6- | | |
| | naphthyridin-4-one | | |
| 168 | 4-oxo-2-(1,1,6- | 347.41 | ¹H NMR (400 MHz, DMSO-d₆) δ (ppm) |
| | trimethylindan-5-yl)-1H- | 348.3 | 11.94 (s, 1H), 8.49 (d, J = 5.8 Hz, 1H), 7.55- |
| | 1,6-naphthyridine-5- | | 7.49 (m, 1H), 7.44 (d, J = 5.8 Hz, 1H), |
| | carboxamide | | 7.29 (s, 1H), 7.23 (d, J = 10.1 Hz, 2H), 6.06 |
| | | | (s, 1H), 2.89 (t, J = 7.2 Hz, 2H), 2.28 (s, |
| | | | 3H), 1.92 (t, J = 7.2 Hz, 2H), 1.26 (s, 6H). |
| 169 | 6-fluoro-2-(4-isopropyl- | 309.38 | ¹H NMR (400 MHz, CD₃OD) δ (ppm) 7.97 |
| | 2,5-dimethyl-phenyl)-1H- | 310.3 | (dd, J = 9.0, 2.8 Hz, 1H), 7.81 (dd, J = 9.2, |
| | quinolin-4-one | | 4.5 Hz, 1H), 7.70-7.62 (m, 1H), 7.26 (d, |
| | | | J = 21.0 Hz, 2H), 6.54 (s, 1H), 3.22 (p, J = 6.9 |
| | | | Hz, 1H), 2.38 (s, 3H), 2.32 (s, 3H), 1.27 (d, |
| | | | J = 6.9 Hz, 6H). |
| 170 | 2-(4-tert-butyl-2-methyl- | 317.38 | ¹H NMR (400 MHz, DMSO-d₆) δ (ppm) |
| | phenyl)-4-oxo-1H-1,6- | 318.1 | 12.34 (s, 1H), 8.72 (d, J = 5.8 Hz, 1H), 7.72 |
| | naphthyridine-5- | | (d, J = 5.8 Hz, 1H), 7.46-7.36 (m, 3H), |
| | carbonitrile | | 6.24 (s, 1H), 2.33 (s, 3H), 1.33 (s, 9H). |
| 171 | 6-fluoro-2-(2-fluoro-4- | 313.34 | ¹H NMR (400 MHz, DMSO-d₆) δ (ppm) |
| | isopropyl-5-methyl- | 314.4 | 11.97 (s, 1H), 7.81-7.69 (m, 2H), 7.60 (td, |
| | phenyl)-1H-quinolin-4- | | J = 8.6, 3.0 Hz, 1H), 7.49 (d, J = 7.8 Hz, |
| | one | | 1H), 7.29 (d, J = 12.2 Hz, 1H), 6.17 (s, 1H), |
| | | | 3.21-3.09 (m, 1H), 2.36 (s, 3H), 1.22 (d, |
| | | | J = 6.8 Hz, 6H) |
| 172 | 6-fluoro-2-(5-fluoro-4- | 313.34 | ¹H NMR (400 MHz, DMSO-d₆) δ (ppm) |
| | isopropyl-2-methyl- | 314.4 | 11.93 (s, 1H), 7.75 (dd, J = 9.3, 2.9 Hz, 1H), |
| | phenyl)-1H-quinolin-4- | | 7.66 (dd, J = 9.2, 4.7 Hz, 1H), 7.59 (td, J = |
| | one | | 8.7, 3.0 Hz, 1H), 7.37 (d, J = 7.6 Hz, 1H), |
| | | | 7.27 (d, J = 10.7 Hz, 1H), 6.02 (s, 1H), 3.21 |
| | | | (hept, J = 7.0 Hz, 1H), 2.27 (s, 3H), 1.26 (d, |
| | | | J = 6.9 Hz, 6H) |
| 173 | 6-methoxy-2-[2-methyl-4- | 376.37 | |
| | (2,2,2-trifluoro-1,1- | 377.3 | |
| | dimethyl-ethyl)phenyl]- | | |
| | 1H-1,5-naphthyridin-4- | | |
| | one | | |
| 174 | 2-[5-chloro-2-methyl-4- | 410.82 | |
| | (2,2,2-trifluoro-1,1- | 411.2 | |
| | dimethyl-ethyl)phenyl]-6- | | |
| | methoxy-1H-1,5- | | |
| | naphthyridin-4-one | | |
| 175 | 2-[5-chloro-2-methyl-4- | 397.79 | |
| | (2,2,2-trifluoro-1,1- | 398 | |
| | dimethyl-ethyl)phenyl]-6- | | |
| | fluoro-1H-quinolin-4-one | | |
| 176 | 2-[5-chloro-2-methyl-4- | 380.79 | ¹H NMR (400 MHz, DMSO-d₆) δ (ppm) |
| | (2,2,2-trifluoro-1,1- | 381.2 | 12.55 (s, 1H), 9.34 (s, 1H), 8.69 (d, J = 6.4 |
| | dimethyl-ethyl)phenyl]- | | Hz, 1H), 7.74 (d, J = 6.4 Hz, 1H), 7.69 (s, |
| | 1H-1,6-naphthyridin-4- | | 1H), 7.64 (s, 1H), 6.35 (s, 1H), 2.37 (s, 3H), |
| | one | | 1.82 (s, 6H). |
| 177 | 2-(4-tert-butyl-2-methyl- | 339.4 | |
| | phenyl)-5-fluoro-6- | 340.3 | |
| | methoxy-1H-quinolin-4- | | |
| | one | | |
| 178 | 2-(4-tert-butyl-2,5- | 353.43 | |
| | dimethyl-phenyl)-5- | 354.3 | |
| | fluoro-6-methoxy-1H- | | |
| | quinolin-4-one | | |
| 179 | 6-fluoro-2-(1,1,7- | 335.41 | ¹H NMR (400 MHz, CD₃OD) δ (ppm) 8.13- |
| | trimethyltetralin-6-yl)-1H- | 336.3 | 8.05 (m, 2H), 7.94-7.87 (m, 1H), 7.45 (s, |
| | quinolin-4-one | | 1H), 7.23 (s, 1H), 7.08 (s, 1H), 2.83 (t, J = |
| | | | 6.3 Hz, 2H), 2.34 (s, 3H), 1.91-1.83 (m, |
| | | | 2H), 1.77-1.71 (m, 2H), 1.34 (s, 6H). |
| 180 | 2-[4-(1-methoxy-1- | 307.39 | ¹H NMR (400 MHz, DMSO-d₆) δ (ppm) |
| | methyl-ethyl)-2-methyl- | 308.5 | 11.77 (s, 1H), 8.11 (dd, J = 8.1, 1.5 Hz, 1H), |
| | phenyl]-1H-quinolin-4- | | 7.66 (ddd, J = 8.3, 6.7, 1.5 Hz, 1H), 7.60 (d, |
| | one | | J = 8.2 Hz, 1H), 7.45-7.39 (m, 2H), 7.39- |
| | | | 7.30 (m, 2H), 5.98 (d, J = 1.5 Hz, 1H), 3.05 |
| | | | (s, 3H), 2.33 (s, 3H), 1.49 (s, 6H) |
| 181 | 2-[5-fluoro-2-methyl-4- | 364.34 | |
| | (2,2,2-trifluoro-1,1- | 365.2 | |
| | dimethyl-ethyl)phenyl]- | | |
| | 1H-1,6-naphthyridin-4- | | |
| | one | | |
| 182 | 6-fluoro-2-[5-fluoro-2- | 381.34 | |
| | methyl-4-(2,2,2-trifluoro- | 382.2 | |
| | 1,1-dimethyl- | | |
| | ethyl)phenyl]-1H- | | |
| | quinolin-4-one | | |
| 183 | 2-[5-fluoro-2-methyl-4- | 394.36 | |
| | (2,2,2-trifluoro-1,1- | 395.2 | |
| | dimethyl-ethyl)phenyl]-6- | | |
| | methoxy-1H-1,5- | | |
| | naphthyridin-4-one | | |
| 184 | 2-(4-tert-butyl-2-methyl- | 336.38 | ¹H NMR (400 MHz, DMSO-d₆) δ (ppm) |
| | phenyl)-4-oxo-1H-1,6- | 337.5 | 12.14 (s, 1H), 8.54 (d, J = 5.8 Hz, 1H), 7.50 |
| | naphthyridine-5- | | (d, J = 5.9 Hz, 1H), 7.44 (s, 1H), 7.42-7.36 |
| | carboxylic acid | | (m, 2H), 6.14 (s, 1H), 2.33 (s, 3H), 1.33 (s, |
| | | | 9H). |
| 185 | 2-[5-fluoro-2-methyl-4- | 389.35 | ¹H NMR (400 MHz, DMSO-d₆) δ (ppm) |
| | (2,2,2-trifluoro-1,1- | 390.3 | 12.42 (s, 1H), 8.73 (d, J = 5.8 Hz, 1H), 7.73 |
| | dimethyl-ethyl)phenyl]-4- | | (d, J = 5.8 Hz, 1H), 7.60 (d, J = 8.1 Hz, 1H), |
| | oxo-1H-1,6- | | 7.43 (d, J = 13.0 Hz, 1H), 6.31 (s, 1H), 2.33 |
| | naphthyridine-5- | | (s, 3H), 1.68 (d, J = 1.7 Hz, 6H). |
| | carbonitrile | | |
| 186 | 2-(6-tert-butyl-2-fluoro-3- | 314.33 | ¹H NMR (400 MHz, DMSO-d₆) δ (ppm) |
| | pyridyl)-6-fluoro-1H- | 315.4 | 7.92 (dd, J = 9.2, 4.7 Hz, 1H), 7.78 (dd, J = |
| | quinolin-4-one | | 9.3, 2.8 Hz, 2H), 7.67 (td, J = 8.7, 3.0 Hz, |
| | | | 1H), 7.51 (s, 1H), 6.71 (s, 1H), 1.37 (s, 9H). |
| 187 | 2-[2,5-dimethyl-4-(2,2,2- | 385.38 | ¹H NMR (400 MHz, DMSO-d₆) δ (ppm) |
| | trifluoro-1,1-dimethyl- | 386.2 | 12.38 (s, 1H), 8.73 (d, J = 5.8 Hz, 1H), 7.74 |
| | ethyl)phenyl]-4-oxo-1H- | | (d, J = 5.8 Hz, 1H), 7.55 (s, 1H), 7.33 (s, |
| | 1,6-naphthyridine-5- | | 1H), 6.26 (d, J = 1.5 Hz, 1H), 2.55 (s, 3H), |
| | carbonitrile | | 2.31 (s, 3H), 1.72 (s, 6H). |
| 188 | 2-[5-chloro-2-methyl-4- | 405.8 | ¹H NMR (400 MHz, DMSO-d₆) δ (ppm) |
| | (2,2,2-trifluoro-1,1- | 406.3 | 12.42 (s, 1H), 8.73 (d, J = 5.8 Hz, 1H), 7.73 |
| | dimethyl-ethyl)phenyl]-4- | | (d, J = 5.8 Hz, 1H), 7.70 (s, 1H), 7.64 (s, |
| | oxo-1H-1,6- | | 1H), 6.32 (s, 1H), 2.34 (s, 3H), 1.80 (s, 6H). |
| | naphthyridine-5- | | |
| | carbonitrile | | |
| 189 | 3-bromo-2-(4-tert-butyl-2- | 414.3 | ¹H NMR (400 MHz, DMSO-d₆) δ (ppm) |
| | methyl-phenyl)-4-oxo-1H- | 416.2 | 12.50 (s, 1H), 8.55 (d, J = 5.8 Hz, 1H), 7.57 |
| | 1,6-naphthyridine-5- | | (s, 1H), 7.48-7.39 (m, 3H), 7.36 (s, 1H), |
| | carboxamide | | 7.31 (d, J = 8.0 Hz, 1H), 2.20 (s, 3H), 1.34 |
| | | | (s, 9H). |
| 190 | 2-[5-chloro-2-methyl-4- | 355.71 | ¹H NMR (400 MHz, DMSO-d₆) δ (ppm) |
| | (trifluoromethyl)phenyl]- | 356.3 | 12.04 (s, 1H), 7.95 (s, 1H), 7.89 (s, 1H), |
| | 6-fluoro-1H-quinolin-4- | | 7.77 (dd, J = 9.3, 2.9 Hz, 1H), 7.70-7.57 |
| | one | | (m, 2H), 6.10 (s, 1H), 2.36 (s, 3H). |
| 191 | 6-fluoro-5-methoxy-2- | 351.41 | ¹H NMR (400 MHz, CD₃OD) δ (ppm) 7.68 |
| | (1,1,6-trimethylindan-5- | 352.3 | (t, J = 9.9 Hz, 1H), 7.49 (dd, J = 9.3, 4.1 Hz, |
| | yl)-1H-quinolin-4-one | | 1H), 7.24 (s, 1H), 7.18 (s, 1H), 6.47 (s, 1H), |
| | | | 4.05 (d, J = 1.4 Hz, 3H), 2.92 (t, J = 7.2 Hz, |
| | | | 2H), 2.32 (s, 3H), 2.02-1.94 (m, 2H), 1.29 |
| | | | (s, 6H). |
| 192 | 2-(6-tert-butyl-4-methyl- | 310.37 | ¹H NMR (400 MHz, CD₃OD) δ (ppm) 8.94- |
| | 3-pyridyl)-6-fluoro-1H- | 311.5 | 8.86 (m, 1H), 8.19-8.13 (m, 1H), 8.11- |
| | quinolin-4-one | | 8.03 (m, 1H), 8.03-7.91 (m, 1H), 7.87- |
| | | | 7.77 (m, 1H), 6.96-6.81 (m, 1H), 2.66 (s, |
| | | | 3H), 1.58 (s, 9H). |
| 193 | 2-(6-tert-butyl-2,5- | 354.42 | ¹H NMR (400 MHz, CD₃OD) δ (ppm) 8.35 |
| | dimethyl-3-pyridyl)-6- | 355.3 | (s, 1H), 7.96 (dd, J = 11.0, 9.4 Hz, 1H), 7.81 |
| | fluoro-5-methoxy-1H- | | (dd, J = 9.4, 4.0 Hz, 1H), 7.11 (s, 1H), 4.19 |
| | quinolin-4-one | | (d, J = 2.1 Hz, 3H), 2.79 (s, 3H), 2.77 (s, |
| | | | 3H), 1.66 (s, 9H). |
| 194 | 2-(1,6-dimethylindazol-5- | 289.33 | ¹H NMR (400 MHz, DMSO-d₆) δ (ppm) |
| | yl)-1H-quinolin-4-one | 290.2 | 13.27 (s, 1H), 8.32-8.22 (m, 1H), 8.15 (s, |
| | | | 1H), 7.96 (s, 1H), 7.94-7.90 (m, 1H), 7.90- |
| | | | 7.84 (m, 1H), 7.70 (s, 1H), 7.59 (ddd, J = |
| | | | 8.2, 6.6, 1.4 Hz, 1H), 6.68 (s, 1H), 4.10 (s, |
| | | | 3H), 2.47 (s, 3H). |
| 195 | 2-(6-tert-butyl-2,5- | 323.39 | ¹H NMR (400 MHz, CD₃OD) δ (ppm) 9.38 |
| | dimethyl-3-pyridyl)-6- | 324.4 | (br s, 1H), 8.74 (br s, 1H), 8.43 (br s, 1H), |
| | oxido-1H-1,6- | | 7.97 (br. s, 1H), 6.64 (s, 1H), 2.85 (s, 3H), |
| | naphthyridin-6-ium-4-one | | 2.78 (s, 3H), 1.67 (s, 9H). |
| 196 | 2-(6-tert-butyl-2,5- | 323.39 | ¹H NMR (400 MHz, CD₃OD) δ (ppm) 8.47 |
| | dimethyl-3-pyridyl)-1,6- | 324.5 | (s, 1H), 7.93 (d, J = 7.5 Hz, 1H), 7.37 (s, |
| | dihydro-1,6- | | 1H), 6.97 (d, J = 7.4 Hz, 1H), 2.86 (s, 3H), |
| | naphthyridine-4,5-dione | | 2.79 (s, 3H), 1.69 (s, 9H). |
| 197 | 2-(1,5-dimethylindol-6- | 288.34 | ¹H NMR (400 MHz, DMSO-d₆) δ (ppm) |
| | yl)-1H-quinolin-4-one | 289 | 12.32 (s, 1H), 8.20 (d, J = 8.1 Hz, 1H), 7.79- |
| | | | 7.71 (m, 2H), 7.61 (s, 1H), 7.56 (s, 1H), |
| | | | 7.47-7.40 (m, 2H), 6.45 (d, J = 3.1 Hz, |
| | | | 1H), 6.32 (s, 1H), 3.83 (s, 3H), 2.40 (s, 3H). |
| 198 | 4-oxo-2-[8- | 366.3 | ¹H NMR (400 MHz, DMSO-d₆) δ (ppm) |
| | (trifluoromethyl)-3- | 367.2 | 12.65 (s, 1H), 9.49 (d, J = 2.4 Hz, 1H), 9.10 |
| | quinolyl]-1H-1,6- | | (d, J = 2.4 Hz, 1H), 8.80 (d, J = 5.8 Hz, 1H), |
| | naphthyridine-5- | | 8.48 (d, J = 8.2 Hz, 1H), 8.35 (d, J = 7.3 Hz, |
| | carbonitrile | | 1H), 7.96-7.86 (m, 2H), 6.90 (s, 1H). |
| 199 | 2-[5-chloro-2-methyl-4- | 354.71 | ¹H NMR (400 MHz, CD₃OD) δ (ppm) 7.90 |
| | (trifluoromethyl)phenyl]- | 355.2 | (s, 1H), 7.88 (d, J = 7.4 Hz, 1H), 7.83 (s, |
| | 1,6-dihydro-1,6- | | 1H), 7.29 (s, 1H), 6.82 (d, J = 7.5 Hz, 1H), |
| | naphthyridine-4,5-dione | | 2.42 (s, 3H). |
| 200 | 2-[2,5-dimethyl-6- | 335.28 | ¹H NMR (400 MHz, CD₃OD) δ (ppm) 8.00 |
| | (trifluoromethyl)-3- | 336.2 | (s, 1H), 7.90 (d, J = 7.5 Hz, 1H), 7.35 (s, |
| | pyridyl]-1,6-dihydro-1,6- | | 1H), 6.83 (d, J = 7.4 Hz, 1H), 2.59 (s, 3H), |
| | naphthyridine-4,5-dione | | 2.57 (s, 3H). |
| 201 | 2-(4-tert-butyl-5-chloro-2- | 370.83 | ¹H NMR (400 MHz, DMSO-d₆) δ (ppm) |
| | methyl-phenyl)-4-oxo-1H- | 371.3 | 12.17 (s, 1H), 8.55 (d, J = 5.8 Hz, 1H), 7.50 |
| | 1,6-naphthyridine-5- | | (d, J = 11.3 Hz, 3H), 6.19 (s, 1H), 2.31 (s, |
| | carboxylic acid | | 3H), 1.49 (s, 9H). |
| 202 | 2-(4-tert-butyl-5-chloro-2- | 326.82 | ¹H NMR (400 MHz, DMSO-d₆) δ (ppm) |
| | methyl-phenyl)-1H-1,6- | 327 | 12.70 (s, 1H), 9.40 (s, 1H), 8.72 (d, J = 6.6, |
| | naphthyridin-4-one | | 1.8 Hz, 1H), 7.80 (d, J = 5.2 Hz, 1H), 7.56 |
| | | | (s, 1H), 7.51 (s, 1H), 6.41 (d, J = 1.6 Hz, |
| | | | 1H), 2.34 (s, 3H), 1.49 (s, 9H). |
| 203 | 2-(4-tert-butyl-5-chloro-2- | 356.85 | ¹H NMR (400 MHz, CDCl₃) δ (ppm) 8.89 (s, |
| | methyl-phenyl)-5- | 357.4 | 1H), 8.37 (s, 1H), 7.56 (s, 1H), 7.54 (s, 1H), |
| | methoxy-1H-1,7- | | 6.88 (s, 1H), 4.18 (s, 3H), 2.39 (s, 3H), 1.54 |
| | naphthyridin-4-one | | (s, 9H). |
| 204 | 2-(4-tert-butyl-5-chloro-2- | 383.87 | ¹H NMR (400 MHz, DMSO-d₆) δ (ppm) |
| | methyl-phenyl)-3-methyl- | 384.3 | 8.45 (d, J = 5.8 Hz, 1H), 7.52 (s, 1H), 7.48 |
| | 4-oxo-1H-1,6- | | (d, J = 6.5 Hz, 1H), 7.42 (s, 1H), 7.38 (d, J = |
| | naphthyridine-5- | | 5.9 Hz, 1H), 7.26 (s, 1H), 2.15 (s, 3H), 1.70 |
| | carboxamide | | (s, 3H), 1.50 (s, 9H). |
| 205 | 2-(4-tert-butyl-5-chloro-2- | 343.81 | ¹H NMR (400 MHz, DMSO-d₆) δ (ppm) |
| | methyl-phenyl)-1,6- | 344.3 | 13.35 (s, 1H), 8.44 (s, 1H), 7.49 (d, J = 5.0 |
| | dihydropyrido[2,3- | | Hz, 1H), 7.42 (s, 1H), 7.31 (s, 1H), 2.35 (d, |
| | d]pyridazine-4,5-dione | | J = 6.2 Hz, 3H), 1.48 (s, 9H). |
| 206 | 2-(4-tert-butyl-5-chloro-2- | 340.85 | ¹H NMR (500 MHz, DMSO-d₆) δ (ppm) |
| | methyl-phenyl)-3-methyl- | 341.3 | 9.25 (d, J = 0.8 Hz, 1H), 8.56 (d, J = 5.8 Hz, |
| | 1H-1,6-naphthyridin-4- | | 1H), 7.52 (d, J = 0.8 Hz, 1H), 7.44 (s, 1H), |
| | one | | 7.38 (dd, J = 5.8, 0.8 Hz, 1H), 2.15 (s, 3H), |
| | | | 1.74 (s, 3H), 1.50 (s, 9H). |
| 207 | 2-(4-tert-butyl-2,5- | 323.4 | ¹H NMR (400 MHz, DMSO-d₆) δ (ppm) |
| | dimethyl-phenyl)-6- | 324.3 | 12.18 (s, 1H), 7.78 (dd, J = 9.3, 3.0 Hz, 1H), |
| | fluoro-1H-quinolin-4-one | | 7.72 (dd, J = 9.1, 4.7 Hz, 1H), 7.62 (td, J = |
| | | | 8.7, 3.0 Hz, 1H), 7.33 (s, 1H), 7.21 (s, 1H), |
| | | | 6.12 (s, 1H), 2.53 (s, 3H), 2.27 (s, 3H), 1.41 |
| | | | (s, 9H). |
| 208 | 5-methoxy-2-[2-methyl-4- | 373.37 | |
| | [1- | 374.5 | |
| | (trifluoromethyl)cyclopropyl] | | |
| | phenyl]-1H-quinolin-4- | | |
| | one | | |
| 209 | 2-(4-tert-butyl-2-methyl- | 305.41 | ¹H NMR (500 MHz, DMSO-d₆) δ (ppm) |
| | phenyl)-8-methyl-1H- | 306.5 | 10.73 (s, 1H), 8.01 (dd, J = 8.1, 1.6 Hz, 1H), |
| | quinolin-4-one | | 7.50 (d, J = 7.1 Hz, 1H), 7.40 (s, 1H), 7.36 |
| | | | (s, 2H), 7.24 (t, J = 7.6 Hz, 1H), 5.96 (s, |
| | | | 1H), 2.53 (s, 3H), 2.34 (s, 3H), 1.34 (s, 9H). |

Method D:

Step 1: A mixture of Intermediate A (1 eq), Intermediate B (1-2 eq, custom or commercial boronic acid or boronic ester), Palladium source (1-5 mol %, e.g. PdCl₂(dppf) or PdCl₂(dtbpf), base (2-3 eq, eg. potassium phosphate) in organic solvent (e.g. dioxane, DMSO, toluene) and water is degassed with bubbling nitrogen and stirred under inert atmosphere at a temperature ranging from room temperature to 120° C. The reaction mixture is filtered and purified via silica gel column chromatography or reverse phase HPLC to obtain the protected intermediate.

Step 2: A solution of the protected intermediate in the appropriate solvent (e.g. toluene, dioxane) is treated with acid (e.g. TFA or HCl) and stirred at either room temperature or elevated temperature (e.g. 70° C.) to transform the nitrile functionality to a carboxamide and to remove the benzyl ether protecting group. The reaction mixture is neutralized and purified via silica gel column chromatography or reverse phase column chromatography (C₁₈) to provide the desired product I.

The following compounds (Table 4) were synthesized using the general Method D using custom or commercially available boronic acids or boronic esters.

TABLE 4

| | | LC/MS | |
|---|---|---|---|
| | | (m/z | |
| | | calc.), | |
| | | Found | |
| Cmpd. No. | Compound Name | M + 1 | NMR (shifts in ppm) |
| 210 | 2-(4-tert-butyl-2,5- | 349.43 | ¹H NMR (400 MHz, DMSO-d₆) δ (ppm) |
| | dimethyl-phenyl)-4- | 350.3 | 11.92 (s, 1H), 8.49 (d, J = 5.8 Hz, 1H), 7.51 |
| | oxo-1H-1,6- | | (s, 1H), 7.44 (d, J = 5.8 Hz, 1H), 7.34 (s, |
| | naphthyridine-5- | | 1H), 7.29 (s, 1H), 7.20 (s, 1H), 6.07 (s, 1H), |
| | carboxamide | | 2.53 (s, 3H), 2.28 (s, 3H), 1.41 (s, 9H). |
| 211 | 2-(4-tert-butyl-2- | 335.4 | ¹H NMR (400 MHz, DMSO-d₆) δ (ppm) |
| | methyl-phenyl)-4- | 336.4 | 11.94 (s, 1H), 8.49 (d, J = 5.8 Hz, 1H), 7.50 |
| | oxo-1H-1,6- | | (s, 1H), 7.45-7.42 (m, 2H), 7.41-7.34 (m, |
| | naphthyridine-5- | | 2H), 7.29 (s, 1H), 6.07 (s, 1H), 2.32 (s, 3H), |
| | carboxamide | | 1.33 (s, 9H). |
| 212 | 2-(5-tert-butyl-2- | 335.4 | |
| | methyl-phenyl)-4- | 336.6 | |
| | oxo-1H-1,6- | | |
| | naphthyridine-5- | | |
| | carboxamide | | |
| 213 | 4-oxo-2-(1,1,7- | 361.44 | ¹H NMR (400 MHz, CD₃OD) δ (ppm) 8.82 |
| | trimethyltetralin-6- | 362.5 | (d, J = 6.0 Hz, 1H), 7.92 (d, J = 6.0 Hz, 1H), |
| | yl)-1H-1,6- | | 7.42 (s, 1H), 7.20 (s, 1H), 6.87 (s, 1H), 2.81 |
| | naphthyridine-5- | | (t, J = 6.3 Hz, 2H), 2.35 (s, 3H), 1.91-1.80 |
| | carboxamide | | (m, 2H), 1.76-1.70 (m, 2H), 1.33 (s, 6H). |
| 214 | 2-[5-fluoro-2- | 407.36 | ¹H NMR (400 MHz, DMSO-d₆) δ (ppm) |
| | methyl-4-(2,2,2- | 408.2 | 12.04 (s, 1H), 8.51 (d, J = 5.8 Hz, 1H), 7.60 |
| | trifluoro-1,1- | | (d, J = 8.1 Hz, 1H), 7.54 (s, 1H), 7.45 (d, J = |
| | dimethyl- | | 5.8 Hz, 1H), 7.40 (d, J = 13.0 Hz, 1H), 7.33 |
| | ethyl)phenyl]-4-oxo- | | (s, 1H), 6.15 (s, 1H), 2.32 (s, 3H), 1.68 (d, |
| | 1H-1,6- | | J = 1.6 Hz, 6H). |
| | naphthyridine-5- | | |
| | carboxamide | | |
| 215 | 2-[2-methyl-4-(2,2,2- | 389.37 | ¹H NMR (400 MHz, DMSO-d₆) δ (ppm) |
| | trifluoro-1,1- | 390.6 | 12.01 (s, 1H), 8.50 (d, J = 5.8 Hz, 1H), 7.60 |
| | dimethyl- | | (s, 1H), 7.57-7.49 (m, 2H), 7.50-7.39 (m, |
| | ethyl)phenyl]-4-oxo- | | 2H), 7.32 (s, 1H), 6.10 (s, 1H), 2.35 (s, 3H), |
| | 1H-1,6- | | 1.60 (s, 6H). |
| | naphthyridine-5- | | |
| | carboxamide | | |
| 216 | 2-(4-tert-butyl-2- | 351.4 | ¹H NMR (400 MHz, DMSO-d₆) δ (ppm) |
| | methoxy-phenyl)-4- | 352.5 | 12.73 (s, 1H), 8.61 (d, J = 6.2 Hz, 1H), 8.10 |
| | oxo-1H-1,6- | | (s, 1H), 7.97 (s, 1H), 7.83 (d, J = 6.2 Hz, |
| | naphthyridine-5- | | 1H), 7.48 (d, J = 8.0 Hz, 1H), 7.22-7.13 |
| | carboxamide | | (m, 2H), 6.46 (s, 1H), 3.88 (s, 3H), 1.35 (s, |
| | | | 9H). |
| 217 | 2-[2,5-dimethyl-4-[1- | 387.36 | ¹H NMR (400 MHz, DMSO-d₆) δ (ppm) |
| | (trifluoromethyl)vinyl] | 388.4 | 12.23 (s, 1H), 8.54 (d, J = 5.9 Hz, 1H), 7.69 |
| | phenyl]-4-oxo-1H- | | (s, 1H), 7.55 (d, J = 6.0 Hz, 1H), 7.51 (s, |
| | 1,6-naphthyridine-5- | | 1H), 7.43 (s, 1H), 7.25 (s, 1H), 6.32 (d, J = |
| | carboxamide | | 1.7 Hz, 1H), 6.19 (s, 1H), 5.81 (s, 1H), 2.29 |
| | | | (d, J = 5.4 Hz, 6H). |
| 218 | 2-(4-tert-butyl-2- | 355.82 | |
| | chloro-phenyl)-4- | 356.3 | |
| | oxo-1H-1,6- | | |
| | naphthyridine-5- | | |
| | carboxamide | | |
| 219 | 2-(4-isopropyl-2,5- | 335.4 | ¹H NMR (400 MHz, DMSO-d₆) δ (ppm) |
| | dimethyl-phenyl)-4- | 336.2 | 11.93 (s, 1H), 8.49 (d, J = 5.8 Hz, 1H), 7.52 |
| | oxo-1H-1,6- | | (s, 1H), 7.45 (d, J = 5.8 Hz, 1H), 7.29 (s, |
| | naphthyridine-5- | | 1H), 7.27 (s, 1H), 7.20 (s, 1H), 6.06 (s, 1H), |
| | carboxamide | | 3.15 (p, J = 6.8 Hz, 1H), 2.33 (s, 3H), 2.28 |
| | | | (s, 3H), 1.22 (d, J = 6.8 Hz, 6H). |
| 220 | 2-[5-methoxy-2- | 419.4 | ¹H NMR (400 MHz, DMSO-d₆) δ (ppm) |
| | methyl-4-(2,2,2- | 420.5 | 12.01 (s, 1H), 8.50 (d, J = 5.8 Hz, 1H), 7.52 |
| | trifluoro-1,1- | | (s, 1H), 7.46 (d, J = 6.5 Hz, 1H), 7.41 (s, |
| | dimethyl- | | 1H), 7.33 (s, 1H), 7.14 (s, 1H), 6.14 (s, 1H), |
| | ethyl)phenyl]-4-oxo- | | 3.84 (s, 3H), 2.26 (s, 3H), 1.66 (s, 6H). [1] |
| | 1H-1,6- | | |
| | naphthyridine-5- | | |
| | carboxamide | | |
| 221 | 2-(6-tert-butyl-2- | 336.39 | ¹H NMR (400 MHz, DMSO-d₆) δ (ppm) |
| | methyl-3-pyridyl)-4- | 337.4 | 12.24 (s, 1H), 8.53 (d, J = 5.8 Hz, 1H), 7.81 |
| | oxo-1H-1,6- | | (d, J = 8.1 Hz, 1H), 7.62 (s, 1H), 7.53 (d, J = |
| | naphthyridine-5- | | 5.9 Hz, 1H), 7.35-7.48 (m, 2H), 6.21 (s, 1H), |
| | carboxamide | | 1.36 (s, 9H). Me signal overlapped with a |
| | | | solvent. |
| 222 | 4-oxo-2-(3,3,6- | 347.41 | ¹H NMR (400 MHz, DMSO-d₆) δ (ppm) |
| | trimethylindan-5-yl)- | 348.3 | 12.46 (s, 1H), 8.58 (d, J = 6.1 Hz, 1H), 7.89 |
| | 1H-1,6- | | (s, 1H), 7.72 (s, 1H), 7.68 (d, J = 6.1 Hz, |
| | naphthyridine-5- | | 1H), 7.23 (d, J = 2.1 Hz, 2H), 6.20 (s, 1H), |
| | carboxamide | | 2.91 (t, J = 7.2 Hz, 2H), 2.27 (s, 3H), 1.92 (t, |
| | | | J = 7.2 Hz, 2H), 1.25 (s, 6H). |
| 223 | 4-oxo-2-(1,1,4- | 347.41 | ¹H NMR (400 MHz, DMSO-d₆) δ (ppm) |
| | trimethylindan-5-yl)- | 348.2 | 11.95 (s, 1H), 8.49 (d, J = 5.7 Hz, 1H), 7.50 |
| | 1H-1,6- | | (s, 1H), 7.43 (d, J = 5.8 Hz, 1H), 7.29 (s, |
| | naphthyridine-5- | | 1H), 7.25 (d, J = 7.7 Hz, 1H), 7.19 (d, J = |
| | carboxamide | | 7.7 Hz, 1H), 6.05 (s, 1H), 2.89 (t, J = 7.2 |
| | | | Hz, 2H), 2.20 (s, 3H), 1.95 (t, J = 7.2 Hz, |
| | | | 2H), 1.27 (s, 6H). |
| 224 | 2-(5-ethyl-2,4- | 321.37 | ¹H NMR (400 MHz, DMSO-d₆-d6) δ (ppm) |
| | dimethyl-phenyl)-4- | 322.5 | 12.21 (s, 1H), 8.54 (d, J = 4.5 Hz, 1H), 7.73 |
| | oxo-1H-1,6- | | (s, 1H), 7.58 (s, 2H), 7.19 (d, J = 6.8 Hz, |
| | naphthyridine-5- | | 2H), 6.15 (s, 1H), 2.63 (q, J = 7.5 Hz, 2H), |
| | carboxamide | | 2.31 (s, 3H), 2.26 (s, 3H), 1.17 (t, J = 7.5 |
| | | | Hz, 3H). |
| 225 | 2-(6-tert-butyl-2,5- | 350.41 | ¹H NMR (400 MHz, CD₃OD) δ (ppm) 8.85 |
| | dimethyl-3-pyridyl)- | 351.5 | (d, J = 6.1 Hz, 1H), 8.50 (s, 1H), 8.15 (d, J = |
| | 4-oxo-1H-1,6- | | 6.3 Hz, 1H), 6.94 (s, 1H), 2.89 (s, 3H), 2.80 |
| | naphthyridine-5- | | (s, 3H), 1.69 (s, 9H). |
| | carboxamide | | |
| 226 | 4-oxo-2-(2,2,6- | 347.41 | ¹H NMR (400 MHz, CD₃OD) δ (ppm) 8.89 |
| | trimethylindan-5-yl)- | 348.4 | (d, J = 6.0 Hz, 1H), 8.01 (d, J = 6.0 Hz, 1H), |
| | 1H-1,6- | | 7.32 (s, 1H), 7.27 (s, 1H), 7.03 (s, 1H), 2.80 |
| | naphthyridine-5- | | (s, 2H), 2.79 (s, 2H), 2.36 (s, 3H), 1.19 (s, |
| | carboxamide | | 6H). |
| 227 | 4-oxo-2-(2,2,7- | 361.44 | ¹H NMR (400 MHz, CD₃OD) δ (ppm) 8.79 |
| | trimethyltetralin-6- | 362.4 | (d, J = 5.9 Hz, 1H), 7.88 (d, J = 5.9 Hz, 1H), |
| | yl)-1H-1,6- | | 7.25 (s, 1H), 7.12 (s, 1H), 6.81 (s, 1H), 2.87 |
| | naphthyridine-5- | | (t, J = 6.8 Hz, 2H), 2.59 (s, 2H), 2.33 (s, |
| | carboxamide | | 3H), 1.63 (t, J = 6.7 Hz, 2H), 1.02 (s, 6H). |
| 228 | 2-(4-tert-butyl-2- | 339.36 | ¹H NMR (400 MHz, CD₃OD) δ (ppm) 8.82 |
| | fluoro-phenyl)-4- | 340.4 | (d, J = 6.0 Hz, 1H), 8.00 (d, J = 6.0 Hz, 1H), |
| | oxo-1H-1,6- | | 7.74 (t, J = 8.1 Hz, 1H), 7.53 (dd, J = 8.2, |
| | naphthyridine-5- | | 1.8 Hz, 1H), 7.46 (dd, J = 13.0, 1.8 Hz, 1H), |
| | carboxamide | | 7.04 (s, 1H), 1.40 (s, 9H). |
| 229 | 2-(2-hydroxy-3- | 332.31 | ¹H NMR (400 MHz, CD₃OD) δ (ppm) 9.19 |
| | quinolyl)-4-oxo-1H- | 333.2 | (s, 1H), 8.94 (d, J = 5.9 Hz, 1H), 8.22 (d, J = |
| | 1,6-naphthyridine-5- | | 5.9 Hz, 1H), 7.97 (d, J = 7.9 Hz, 1H), 7.82 |
| | carboxamide | | (s, 1H), 7.81-7.76 (m, 1H), 7.50 (d, J = 8.3 |
| | | | Hz, 1H), 7.44 (t, J = 7.7 Hz, 1H). |
| 230 | 4-oxo-2-(1,1,4,7- | 361.44 | ¹H NMR (400 MHz, DMSO-d₆-d6) δ (ppm) |
| | tetramethylindan-5- | 362.5 | 12.29 (s, 1H), 8.56 (d, J = 6.0 Hz, 1H), 7.79 |
| | yl)-1H-1,6- | | (s, 1H), 7.63 (s, 1H), 7.58 (d, J = 6.0 Hz, |
| | naphthyridine-5- | | 1H), 7.03 (s, 1H), 6.16 (s, 1H), 2.81 (d, J = |
| | carboxamide | | 7.3 Hz, 2H), 2.38 (s, 3H), 2.15 (s, 3H), 1.92 |
| | | | (t, J = 7.3 Hz, 2H), 1.34 (s, 6H). |
| 231 | 2-(4-chloro-2,5- | 327.76 | ¹H NMR (400 MHz, CD₃OD) δ (ppm) 8.86 |
| | dimethyl-phenyl)-4- | 328.2 | (d, J = 6.0 Hz, 1H), 7.97 (d, J = 6.0 Hz, 1H), |
| | oxo-1H-1,6- | | 7.49 (s, 1H), 7.47 (s, 1H), 6.93 (s, 1H), 2.44 |
| | naphthyridine-5- | | (s, 3H), 2.36 (s, 3H). |
| | carboxamide | | |
| 232 | 2-[4-isopropyl-2- | 389.37 | ¹H NMR (400 MHz, CD₃OD) δ (ppm) 8.85 |
| | methyl-5- | 390.5 | (d, J = 6.0 Hz, 1H), 7.95 (d, J = 6.0 Hz, 1H), |
| | (trifluoromethyl) | | 7.81 (s, 1H), 7.69 (s, 1H), 6.94 (s, 1H), 3.45- |
| | phenyl]-4-oxo-1H-1,6- | | 3.35 (m, 1H), 2.47 (s, 3H), 1.34 (d, J = 6.8 |
| | naphthyridine-5- | | Hz, 6H). |
| | carboxamide | | |
| 233 | 4-oxo-2-(3,3,7- | 361.44 | ¹H NMR (400 MHz, CD₃OD) δ (ppm) 8.77 |
| | trimethyltetralin-6- | 362.5 | (d, J = 6.0 Hz, 1H), 7.86 (d, J = 6.0 Hz, 1H), |
| | yl)-1H-1,6- | | 7.18 (s, 2H), 6.77 (s, 1H), 2.88 (t, J = 6.9 |
| | naphthyridine-5- | | Hz, 2H), 2.58 (s, 2H), 2.33 (s, 3H), 1.63 (t, J = |
| | carboxamide | | 6.8 Hz, 2H), 1.01 (s, 6H). |
| 234 | 2-(5-isopropyl-2,4- | 335.4 | ¹H NMR (400 MHz, CD₃OD) δ (ppm) 8.91 |
| | dimethyl-phenyl)-4- | 336.4 | (d, J = 5.9 Hz, 1H), 8.04 (d, J = 6.0 Hz, 1H), |
| | oxo-1H-1,6- | | 7.38 (s, 1H), 7.24 (s, 1H), 7.02 (s, 1H), 3.23 |
| | naphthyridine-5- | | (sept, J = 6.9 Hz, 1H), 2.41 (s, 3H), 2.33 (s, |
| | carboxamide | | 3H), 1.27 (d, J = 6.8 Hz, 6H). |
| 235 | 4-oxo-2-[8- | 384.31 | ¹H NMR (400 MHz, DMSO-d₆) δ (ppm) |
| | (trifluoromethyl)-3- | 385.2 | 9.53 (s, 1H), 9.13 (s, 1H), 8.61 (d, J = 5.9 |
| | quinolyl]-1H-1,6- | | Hz, 1H), 8.47 (d, J = 8.2 Hz, 1H), 8.33 (d, J = |
| | naphthyridine-5- | | 7.1 Hz, 1H), 7.89 (t, J = 7.8 Hz, 1H), 7.76 |
| | carboxamide | | (s, 2H), 6.89 (s, 1H), 4.28 (s, 2H). |
| 236 | 4-oxo-2-(1,1,4,4,7- | 389.49 | ¹H NMR (400 MHz, CD₃OD) δ (ppm) 8.79 |
| | pentamethyltetralin- | 390.5 | (d, J = 5.9 Hz, 1H), 7.87 (d, J = 5.9 Hz, 1H), |
| | 6-yl)-1H-1,6- | | 7.44 (s, 1H), 7.39 (s, 1H), 6.78 (s, 1H), 2.33 |
| | naphthyridine-5- | | (s, 3H), 1.75 (s, 4H), 1.33 (s, 6H), 1.32 (s, |
| | carboxamide | | 6H). |
| 237 | 2-[4-tert-butyl-2- | 403.4 | ¹H NMR (400 MHz, CD₃OD) δ (ppm) 8.55 |
| | methyl-5- | 404.45 | (d, J = 5.9 Hz, 1H), 7.80 (s, 1H), 7.77 (s, |
| | (trifluoromethyl) | | 1H), 7.54 (d, J = 6.1 Hz, 1H), 6.32 (s, 1H), |
| | phenyl]-4-oxo-1H-1,6- | | 2.43 (s, 3H), 1.51 (s, 9H). |
| | naphthyridine-5- | | |
| | carboxamide | | |
| 238 | 2-[4-tert-butyl-2- | 403.4 | |
| | methyl-3- | 404.3 | |
| | (trifluoromethyl) | | |
| | phenyl]-4-oxo-1H-1,6- | | |
| | naphthyridine-5- | | |
| | carboxamide | | |
| 239 | 4-oxo-2-(1,1,4,4- | 375.46 | ¹H NMR (400 MHz, CDCl₃) δ (ppm) 9.01 (s, |
| | tetramethyltetralin-6- | 376.6 | 1H), 8.79 (s, 1H), 8.21 (s, 1H), 8.04 (d, J = |
| | yl)-1H-1,6- | | 8.0 Hz, 1H), 7.55 (d, J = 8.5 Hz, 1H), 7.44 |
| | naphthyridine-5- | | (s, 1H), 7.26 (s, 1H), 6.47 (s, 1H), 1.74 (s, |
| | carboxamide | | 4H), 1.43 (s, 6H), 1.33 (s, 6H). |
| 240 | 2-(4-tert-butyl-2- | 353.39 | ¹H NMR (400 MHz, DMSO-d₆) δ (ppm) |
| | fluoro-6-methyl- | 354.1 | 12.10 (br. s., 1H), 8.50 (d, J = 5.9 Hz, 1H), |
| | phenyl)-4-oxo-1H- | | 7.53 (br. s., 1H), 7.40 (d, J = 5.9 Hz, 1H), |
| | 1,6-naphthyridine-5- | | 7.30 (s, 2H), 7.25 (d, J = 11.7 Hz, 1H), 6.13 |
| | carboxamide | | (s, 1H), 2.28 (s, 3H), 1.32 (s, 9H). |
| 241 | 2-(4-tert-butyl-5- | 377.48 | ¹H NMR (400 MHz, DMSO-d₆) δ (ppm) |
| | isopropyl-2-methyl- | 378.4 | 11.94 (s, 1H), 8.48 (d, J = 5.8 Hz, 1H), 7.49 |
| | phenyl)-4-oxo-1H- | | (s, 1H), 7.43 (d, J = 5.8 Hz, 1H), 7.32 (s, |
| | 1,6-naphthyridine-5- | | 1H), 7.30-7.23 (m, 2H), 6.04 (s, 1H), 3.58 |
| | carboxamide | | (p, J = 6.7 Hz, 1H), 2.26 (s, 3H), 1.42 (s, |
| | | | 9H), 1.24 (d, J = 6.5 Hz, 6H). |
| 242 | 2-(4-tert-butyl-5- | 375.46 | ¹H NMR (400 MHz, CD₃OD) δ (ppm) 8.52 |
| | cyclopropyl-2- | 376.4 | (d, J = 5.9 Hz, 1H), 7.51 (d, J = 5.9 Hz, 1H), |
| | methyl-phenyl)-4- | | 7.37 (s, 1H), 6.90 (s, 1H), 6.25 (s, 1H), 2.44- |
| | oxo-1H-1,6- | | 2.33 (m, 1H), 2.30 (s, 3H), 1.54 (s, 9H), |
| | naphthyridine-5- | | 1.10-1.01 (m, 2H), 0.83-0.78 (m, 2H). |
| | carboxamide | | |
| 243 | 2-(4-tert-butyl-2- | 367.42 | ¹H NMR (400 MHz, CDCl₃) δ (ppm) 15.43 |
| | fluoro-3,6-dimethyl- | 368.2 | (s, 1H), 9.00 (br. s, 1H), 8.65 (d, J = 5.6 Hz, |
| | phenyl)-4-oxo-1H- | | 1H), 8.16 (d, J = 5.6 Hz, 1H), 7.16 (s, 1H), |
| | 1,6-naphthyridine-5- | | 7.11 (s, 1H), 6.16 (br. s., 1H), 2.44 (d, J = |
| | carboxamide | | 3.4 Hz, 3H), 2.22 (s, 3H), 1.46 (s, 9H). |
| 244 | 2-[5-chloro-2- | 381.05 | ¹H NMR (400 MHz, DMSO-d₆) δ (ppm) |
| | methyl-4- | 382.2 | 12.47 (s, 1H), 8.57 (d, J = 6.0 Hz, 1H), 7.96 |
| | (trifluoromethyl) | | (s, 1H), 7.89 (s, 1H), 7.78 (s, 1H), 7.65- |
| | phenyl]-4-oxo-1H-1,6- | | 7.54 (m, 2H), 6.30 (s, 1H), 2.39 (s, 3H) |
| | naphthyridine-5- | | |
| | carboxamide | | |
| 245 | 2-[6-tert-butyl-2- | 404.16 | H NMR (400 MHz, DMSO-d₆) δ (ppm) |
| | methyl-5- | 405.1 | 12.06 (br. s, 1H), 8.52 (d, J = 5.6 Hz, 1H), |
| | (trifluoromethyl)-3- | | 8.22 (s, 1H), 7.53 (br. s., 1H), 7.42 (d, J = |
| | pyridyl]-4-oxo-1H- | | 5.9 Hz, 1H), 7.32 (br. s., 1H), 6.30-6.27 |
| | 1,6-naphthyridine-5- | | (m, 1H), 2.57 (s, 3H), 1.46 (s, 9H). ¹⁹F NMR |
| | carboxamide | | (377 MHz, DMSO-d₆) δ (ppm) −52.91 (s, 3F |
| 246 | 2-(5-tert-butyl-3,6- | 351.17 | ¹H NMR (400 MHz, CDCl₃) δ (ppm) 15.40 |
| | dimethyl-pyrazin-2- | 352.2 | (br. s, 1H), 8.99 (br. s, 1H), 8.66 (d, J = 5.6 |
| | yl)-4-oxo-1H-1,6- | | Hz, 1H), 8.17 (d, J = 5.4 Hz, 1H), 7.69 (s, |
| | naphthyridine-5- | | 1H), 6.13 (br. s, 1H), 2.81 (s, 3H), 2.79 (s, |
| | carboxamide | | 3H), 1.50 (s, 9H). |
| 247 | 2-(4-(tert-butyl)-2,6- | 357.13 | ¹H NMR (400 MHz, DMSO-d₆) δ (ppm) |
| | difluorophenyl)-4- | 358.1 | 12.24 (s, 1H), 8.52 (d, J = 5.9 Hz, 1H), 7.53 |
| | hydroxy-1,6- | | (br. s., 1H), 7.44-7.35 (m, 3H), 7.32 (br. s., |
| | naphthyridine-5- | | 1H), 6.26 (s, 1H), 1.33 (s, 9H). |
| | carboxamide | | |
| 248 | 2-[2,5-dimethyl-4- | 403.15 | ¹H NMR (400 MHz, DMSO-d₆) δ (ppm) |
| | (2,2,2-trifluoro-1,1- | 404.2 | 11.97 (s, 1H), 8.50 (d, J = 5.8 Hz, 1H), 7.52 |
| | dimethyl- | | (d, J = 10.5 Hz, 2H), 7.44 (d, J = 5.8 Hz, |
| | ethyl)phenyl]-4-oxo- | | 1H), 7.29 (s, 2H), 6.09 (s, 1H), 2.55 (s, 3H), |
| | 1H-1,6- | | 2.30 (s, 3H), 1.72 (s, 6H). |
| | naphthyridine-5- | | |
| | carboxamide | | |
| 249 | 2-[4-(5-carbamoyl-4- | 339.09 | ¹H NMR (400 MHz, DMSO-d₆) δ (ppm) |
| | oxo-1H-1,6- | 400.5 | 8.56 (dd, J = 6.0, 1.9 Hz, 1H), 7.80 (s, 1H), |
| | naphthyridin-2-yl)-2- | | 7.59 (t, J = 5.7 Hz, 2H), 7.54 (d, J = 1.9 Hz, |
| | chloro-5-methyl- | | 2H), 6.26 (d, J = 3.5 Hz, 1H), 2.34 (s, 3H), |
| | phenyl]-2-methyl- | | 1.57 (s, 6H). |
| | propanoic acid | | |
| 250 | 2-(5-chloro-4- | 367.11 | ¹H NMR (400 MHz, CD₃OD) δ 8.54 (d, J = |
| | cyclobutyl-2-methyl- | 368.1 | 6.0 Hz, 1H), 7.53 (d, J = 5.9 Hz, 1H), 7.43 |
| | phenyl)-4-oxo-1H- | | (s, 1H), 7.40 (s, 1H), 6.28 (s, 1H), 3.85 |
| | 1,6-naphthyridine-5- | | (quin, J = 8.8 Hz, 1H), 2.52-2.41 (m, 2H), |
| | carboxamide | | 2.36 (s, 3H), 2.26-2.05 (m, 3H), 1.94-1.85 |
| | | | (m, 1H). |
| 251 | 2-[5-chloro-2- | 367.11 | ¹H NMR (400 MHz, DMSO-d₆) δ 11.99 (s, |
| | methyl-4-(1- | 368.3 | 1H), 8.50 (d, J = 5.8 Hz, 1H), 7.60-7.52 |
| | methylcyclopropyl) | | (m, 1H), 7.50 (s, 1H), 7.47-7.41 (m, 2H), |
| | phenyl]-4-oxo-1H-1,6- | | 7.32 (s, 1H), 6.11 (s, 1H), 2.27 (s, 3H), 1.35 |
| | naphthyridine-5- | | (s, 3H), 0.89-0.77 (m, 4H). |
| | carboxamide | | |
| 252 | 2-[5-chloro-4-(4,4- | 445.14 | ¹H NMR (400 MHz, DMSO-d₆) δ 11.98 (s, |
| | difluoro-1-methyl- | 446.3 | 1H), 8.49 (d, J = 5.8 Hz, 1H), 7.57-7.48 |
| | cyclohexyl)-2- | | (m, 3H), 7.44 (d, J = 5.8 Hz, 1H), 7.30 (s, |
| | methyl-phenyl]-4- | | 1H), 6.13 (s, 1H), 2.47-2.39 (m, 2H), 2.33 |
| | oxo-1H-1,6- | | (s, 3H), 2.17-2.04 (m, 2H), 2.04-1.94 (m, |
| | naphthyridine-5- | | 2H), 1.94-1.79 (m, 2H), 1.45 (s, 3H). |
| | carboxamide | | |
| 253 | 2-[5-chloro-2- | 395.14 | ¹H NMR (400 MHz, DMSO-d₆) δ 11.97 (s, |
| | methyl-4-(1- | 396.3 | 1H), 8.50 (d, J = 5.8 Hz, 1H), 7.60-7.37 |
| | methylcyclopentyl) | | (m, 4H), 7.30 (s, 1H), 6.12 (s, 1H), 2.30 (s, |
| | phenyl]-4-oxo-1H-1,6- | | 3H), 2.17-2.05 (m, 2H), 2.02-1.88 (m, |
| | naphthyridine-5- | | 2H), 1.84-1.63 (m, 4H), 1.33 (s, 3H). |
| | carboxamide | | |
| 254 | 2-[5-chloro-2- | 381.12 | ¹H NMR (400 MHz, CD₃OD) 8 8.54 (d, J = |
| | methyl-4-(1- | 382.3 | 5.4 Hz, 1H), 7.53 (d, J = 5.9 Hz, 1H), 7.39 |
| | methylcyclobutyl) | | (s, 1H), 7.16 (s, 1H), 6.28 (s, 1H), 2.55- |
| | phenyl]-4-oxo-1H-1,6- | | 2.44 (m, 2H), 2.33 (s, 3H), 2.29-2.13 (m, |
| | naphthyridine-5- | | 3H), 1.87-1.77 (m, 1H), 1.56 (s, 3H). |
| | carboxamide | | |
| 255 | 2-[5-chloro-2- | 421.08 | ¹H NMR (400 MHz, DMSO-d₆) δ 12.03 (br. |
| | methyl-4-[1- | 422.05 | s, 1H), 8.51 (d, J = 5.5 Hz, 1H), 7.65 (s, |
| | (trifluoromethyl) | | 1H), 7.63 (s, 1H), 7.53 (br. s, 1H), 7.43 (d, |
| | cyclopropyl]phenyl]-4- | | J = 5.7 Hz, 1H), 7.32 (br. s, 1H), 6.13 (s, 1H), |
| | oxo-1H-1,6- | | 2.31 (s, 3H), 1.57-1.51 (m, 2H), 1.27-1.21 |
| | naphthyridine-5- | | (m, 2H). |
| | carboxamide | | |
| 256 | 2-[5-fluoro-2- | 379.17 | ¹H NMR (400 MHz, CD₃OD) δ 8.82 (d, J = |
| | methyl-4-(1- | 380.1 | 6.0 Hz, 1H), 7.96-7.85 (m, 1H), 7.40 (d, |
| | methylcyclopentyl) | | J = 7.8 Hz, 1H), 7.26 (d, J = 11.7 Hz, 1H), |
| | phenyl]-4-oxo-1H-1,6- | | 6.87 (s, 1H), 2.36 (s, 3H), 2.05-1.91 (m, |
| | naphthyridine-5- | | 4H), 1.90-1.69 (m, 4H), 1.32 (s, 3H). |
| | carboxamide | | |
| 257 | 2-[5-chloro-4-(3,3- | 417.11 | ¹H NMR (400 MHz, DMSO-d₆) δ 11.99 (s, |
| | difluoro-1-methyl- | 418.3 | 1H), 8.56-8.47 (m, 1H), 7.54 (s, 1H), 7.52 |
| | cyclobutyl)-2- | | (s, 1H), 7.44 (d, J = 5.8 Hz, 1H), 7.38 (s, |
| | methyl-phenyl]-4- | | 1H), 7.31 (s, 1H), 6.12 (d, J = 1.6 Hz, 1H), |
| | oxo-1H-1,6- | | 3.19-2.99 (m, 2H), 2.96-2.81 (m, 2H),2.31 |
| | naphthyridine-5- | | (s, 3H), 1.54 (s, 3H). |
| | carboxamide | | |
| 258 | 2-(5-chloro-2- | 389.09 | ¹H NMR (400 MHz, DMSO-d₆) δ 12.11 (s, |
| | methyl-4-phenyl- | 390.2 | 1H), 8.53 (d, J = 5.8 Hz, 1H), 7.69 (s, 1H), |
| | phenyl)-4-oxo-1H- | | 7.57 (s, 1H), 7.55-7.43 (m, 7H), 7.36 (s, |
| | 1,6-naphthyridine-5- | | 1H), 6.21 (s, 1H), 2.34 (s, 3H). |
| | carboxamide | | |
| 259 | 2-(5-chloro-4- | 381.12 | ¹H NMR (400 MHz, CD₃OD) δ 8.54 (d, J = |
| | cyclopentyl-2- | 382.2 | 5.9 Hz, 1H), 7.52 (d, J = 6.0 Hz, 1H), 7.45 |
| | methyl-phenyl)-4- | | (s, 1H), 7.39 (s, 1H), 6.28 (s, 1H), 3.48 (p, J = |
| | oxo-1H-1,6- | | 8.4 Hz, 1H), 2.33 (s, 3H), 2.18-2.06 (m, |
| | naphthyridine-5- | | 2H), 1.94-1.81 (m, 2H), 1.83-1.71 (m, |
| | carboxamide | | 2H), 1.71-1.57 (m, 2H). |
| 260 | 2-[5-chloro-2- | 437.08 | ¹H NMR (400 MHz, DMSO-d₆) |
| | methyl-4-[2- | 438.1 | δ 12.04 (br. s, 1H), 8.51 (br. s, 1H), 7.66 (s, |
| | (trifluoromethyl)oxetan- | | 1H), 7.59-7.49 (m, 2H), 7.44 (d, J = 5.3 |
| | 2-yl]phenyl]-4- | | Hz, 1H), 7.32 (br. s, 1H), 6.17 (s, 1H), 4.83- |
| | oxo-1H-1,6- | | 4.76 (m, 1H), 4.61-4.53 (m, 1H), 3.30- |
| | naphthyridine-5- | | 3.26 (overlapped with water, m, 2H), 2.34 |
| | carboxamide | | (s, 3H). |
| 261 | 2-[4-(1- | 379.11 | ¹H NMR (400 MHz, DMSO-d₆) δ 11.95 (s, |
| | bicyclo[1.1.1]pentanyl)- | 380.2 | 1H), 8.50 (d, J = 5.8 Hz, 1H), 7.51 (s, 1H), |
| | 5-chloro-2- | | 7.46 (s, 1H), 7.43 (d, J = 5.8 Hz, 1H), 7.30 |
| | methyl-phenyl]-4- | | (s, 1H), 7.26 (s, 1H), 6.11 (s, 1H), 2.64- |
| | oxo-1H-1,6- | | 2.58 (m, 1H), 2.30-2.23 (m, 9H). |
| | naphthyridine-5- | | |
| | carboxamide | | |
| 262 | 2-[5-chloro-4-(2,2- | 443.12 | ¹H NMR (400 MHz, CD₃OD) δ 8.77 (d, J = |
| | difluorospiro[3.3] | 444.0 | 6.0 Hz, 1H), 7.88 (d, J = 5.6 Hz, 1H), 7.52 |
| | heptan-6-yl)-2-methyl- | | (s, 1H), 7.41 (s, 1H), 6.74 (s, 1H), 3.77 (p, |
| | phenyl]-4-oxo-1H- | | J = 8.8 Hz, 1H), 2.82-2.72 (m, 2H), 2.68- |
| | 1,6-naphthyridine-5- | | 2.48 (m, 4H), 2.38 (s, 3H), 2.37-2.30 (m, |
| | carboxamide | | 2H). |
| 263 | 2-(5-chloro-2- | 393.12 | ¹H NMR (400 MHz, DMSO-d₆) δ 11.97 (s, |
| | methyl-4- | 394.3 | 1H), 8.50 (d, J = 5.8 Hz, 1H), 7.56 (s, 1H), |
| | spiro[2.3]hexan-5-yl- | | 7.52 (d, J = 3.9 Hz, 2H), 7.45 (d, J = 5.8 Hz, |
| | phenyl)-4-oxo-1H- | | 1H), 7.30 (s, 1H), 6.12 (s, 1H), 3.96 (p, J = |
| | 1,6-naphthyridine-5- | | 8.5 Hz, 1H), 2.48-2.36 (m, 4H), 2.34 (s, |
| | carboxamide | | 3H), 0.63-0.53 (m, 2H), 0.49-0.40 (m, |
| | | | 2H). |
| 264 | 2-[4-(1- | 393.12 | ¹H NMR (400 MHz, CD₃OD) δ 8.54 (d, J = |
| | bicyclo[3.1.0]hexanyl)- | 394.21 | 5.9 Hz, 1H), 7.53 (d, J = 5.9 Hz, 1H), 7.46- |
| | 5-chloro-2-methyl- | | 7.38 (m, 2H), 6.27 (s, 1H), 2.31 (s, 3H), 2.18- |
| | phenyl]-4-oxo-1H- | | 2.07 (m, 1H), 2.05-2.01 (m, 1H), 1.95- |
| | 1,6-naphthyridine-5- | | 1.81 (m, 2H), 1.82-1.67 (m, 1H), 1.64- |
| | carboxamide | | 1.53 (m, 1H), 1.46-1.35 (m, 1H), 0.95- |
| | | | 0.87 (m, 1H), 0.78-0.70 (m, 1H). |
| 265 | 2-(5-chloro-2- | 407.14 | ¹H NMR (400 MHz, CD₃OD) δ 8.63 (d, J = |
| | methyl-4-norbornan- | 408.35 | 6.0 Hz, 1H), 7.65 (d, J = 6.0 Hz, 1H), 7.50 |
| | 2-yl-phenyl)-4-oxo- | | (s, 1H), 7.38 (s, 1H), 6.47 (s, 1H), 3.09 (dd, |
| | 1H-1,6- | | J = 9.0, 5.7 Hz, 1H), 2.44-2.38 (m, 2H), |
| | naphthyridine-5- | | 2.35 (s, 3H), 1.93 (ddd, J = 12.0, 9.1, 2.3 |
| | carboxamide | | Hz, 1H), 1.76-1.55 (m, 4H), 1.51-1.42 (m, |
| | | | 1H), 1.41-1.29 (m, 2H). |
| 266 | 2-[3-chloro-2-fluoro- | 441.09 | ¹H NMR (400 MHz, DMSO-d₆) δ 12.18 (s, |
| | 6-methyl-4-(2,2,2- | 442.4 | 1H), 8.52 (d, J = 5.8 Hz, 1H), 7.59 (s, 1H), |
| | trifluoro-1,1- | | 7.55 (s, 1H), 7.39 (d, J = 5.8 Hz, 1H), 7.33 |
| | dimethyl- | | (s, 1H), 6.23 (s, 1H), 2.31 (s, 3H), 1.82 (s, |
| | ethyl)phenyl]-4-oxo- | | 6H). |
| | 1H-1,6- | | |
| | naphthyridine-5- | | |
| | carboxamide | | |
| 267 | 2-[4-(1- | 421.16 | ¹H NMR (400 MHz, DMSO-d₆) δ 11.99 (br |
| | bicyclo[2.2.2]octanyl)- | 422.2 | s, 1H), 8.48 (d, J = 5.9 Hz, 1H), 7.56-7.48 |
| | 5-chloro-2-methyl- | | (m, 1H), 7.45 (s, 1H), 7.43 (d, J = 5.9 Hz, |
| | phenyl]-4-oxo-1H- | | 1H), 7.36 (s, 1H), 7.30 (br s, 1H), 6.12 (s, |
| | 1,6-naphthyridine-5- | | 1H), 2.29 (s, 3H), 2.08-1.97 (m, 6H), 1.72- |
| | carboxamide | | 1.64 (m, 7H). |
| 268 | 2-[5-chloro-4-(2,2- | 405.11 | ¹H NMR (400 MHz, DMSO-d₆) δ 12.16 (br |
| | difluoro-1,1- | 406.4 | s, 1H), 8.53 (d, J = 5.8 Hz, 1H), 7.64 (br s, |
| | dimethyl-ethyl)-2- | | 1H), 7.59 (s, 1H), 7.56 (s, 1H), 7.51 (d, J = |
| | methyl-phenyl]-4- | | 5.9 Hz, 1H), 7.45 (br s, 1H), 6.80 (t, J = 56.6 |
| | oxo-1H-1,6- | | Hz, 1H), 6.20 (s, 1H), 2.33 (s, 3H), 1.56 (s, |
| | naphthyridine-5- | | 6H). |
| | carboxamide | | |
| 269 | 2-[5-chloro-2- | 397.04 | ¹H-NMR (400 MHz, DMSO-d₆) δ 12.03 (s, |
| | methyl-4- | 398.09 | 1H), 8.51 (d, J = 6.1 Hz, 1H), 7.86 (s, 1H), |
| | (trifluoromethoxy) | | 7.69 (s, 1H), 7.53 (br s, 1H), 7.43 (d, J = 5.3 |
| | phenyl]-4-oxo-1H-1,6- | | Hz, 1H), 7.32 (br s, 1H), 6.17 (s, 1H), 2.34 |
| | naphthyridine-5- | | (s, 3H). |
| | carboxamide | | |
| 270 | 2-[5-chloro-2- | 449.11 | ¹H NMR (400 MHz, CDCl₃) δ 15.52 (s, 1H), |
| | methyl-4-[1- | 450.1 | 8.98 (br s, 1H), 8.64 (d, J = 5.4 Hz, 1H), |
| | (trifluoromethyl) | | 8.12 (d, J = 5.4 Hz, 1H), 7.56 (s, 1H), 7.40 |
| | cyclopentyl]phenyl]-4- | | (s, 1H), 7.18 (s, 1H), 6.15 (br s, 1H), 2.87- |
| | oxo-1H-1,6- | | 2.69 (m, 2H), 2.40 (s, 3H), 2.36-2.22 (m, |
| | naphthyridine-5- | | 2H), 1.99-1.73 (m, 4H). |
| | carboxamide | | |
| 271 | 2-[4-(3- | 393.12 | ¹H NMR (400 MHz, CD₃OD) δ 8.54 (d, J = |
| | bicyclo[3.1.0]hexanyl)- | 394.1 | 6.1 Hz, 1H), 7.52 (d, J = 5.9 Hz, 1H), 7.44 |
| | 5-chloro-2-methyl- | | (s, 1H), 7.34 (s, 1H), 6.28 (s, 1H), 3.93 (tt, J = |
| | phenyl]-4-oxo-1H- | | 9.4, 6.5 Hz, 1H), 2.55-2.41 (m, 2H), 2.32 |
| | 1,6-naphthyridine-5- | | (s, 3H), 1.76 (dd, J = 13.7, 6.6 Hz, 2H), 1.51- |
| | carboxamide | | 1.40 (m, 2H), 0.78-0.67 (m, 1H), 0.32 |
| | | | (app. q, J = 4.0 Hz, 1H). |
| 272 | 2-(5-chloro-2- | 385.1 | ¹H-NMR (400 MHz, DMSO-d₆) δ 12.00 (br |
| | methyl-4- | 386.12 | s, 1H), 8.50 (d, 1H, J = 5.7 Hz), 7.52 (br s, |
| | trimethylsilyl- | | 1H), 7.51 (s, 1H), 7.49 (s, 1H), 7.44 (d, 1H, |
| | phenyl)-4-oxo-1H- | | J = 5.4 Hz), 7.30 (s, 1H), 6.14 (s, 1H), 2.30 |
| | 1,6-naphthyridine-5- | | (s, 3H), 0.40 (s, 9H) |
| | carboxamide | | |
| 273 | 2-(5-chloro-2- | 390.09 | |
| | methyl-6-phenyl-3- | 391.5 | |
| | pyridyl)-4-oxo-1H- | | |
| | 1,6-naphthyridine-5- | | |
| | carboxamide | | |
| 274 | 2-[6-(1- | 422.15 | ¹H NMR (400 MHz, CD₃OD) δ 8.55 (d, J = |
| | bicyclo[2.2.2]octanyl)- | 423.2 | 5.9 Hz, 1H), 7.80 (s, 1H), 7.53 (br d, J = 5.9 |
| | 5-chloro-2-methyl- | | Hz, 1H), 6.35 (br s, 1H), 2.51 (s, 3H), 2.22- |
| | 3-pyridyl]-4-oxo-1H- | | 2.09 (m, 6H), 1.79-1.66 (m, 7H). |
| | 1,6-naphthyridine-5- | | |
| | carboxamide | | |
| 275 | 2-(5-chloro-6- | 406.12 | ¹H NMR (400 MHz, CD₃OD) δ 8.54 (d, J = |
| | dispiro[2.0.24.13] | 407.1 | 5.9 Hz, 1H), 7.80 (s, 1H), 7.52 (d, J = 5.9 |
| | heptan-7-yl-2-methyl-3- | | Hz, 1H), 6.35 (s, 1H), 3.10 (s, 1H), 2.50 (s, |
| | pyridyl)-4-oxo-1H- | | 3H), 1.11-1.02 (m, 2H), 1.04-0.95 (m, |
| | 1,6-naphthyridine-5- | | 2H), 0.86-0.77 (m, 4H). |
| | carboxamide | | |
| 276 | 2-[6-(1- | 380.1 | ¹H NMR (400 MHz, CD₃OD) δ 8.88 (d, J = |
| | bicyclo[1.1.1]pentanyl)- | 381.4 | 6.1 Hz, 1H), 8.02 (d, J = 6.1 Hz, 1H), 7.99 |
| | 5-chloro-2- | | (s, 1H), 7.02 (s, 1H), 2.59 (s, 1H), 2.57 (s, |
| | methyl-3-pyridyl]-4- | | 3H), 2.39 (s, 6H). |
| | oxo-1H-1,6- | | |
| | naphthyridine-5- | | |
| | carboxamide | | |
| 277 | 2-[6-(1- | 394.12 | |
| | bicyclo[2.1.1]hexanyl)- | 395.1 | |
| | 5-chloro-2-methyl- | | |
| | 3-pyridyl]-4-oxo-1H- | | |
| | 1,6-naphthyridine-5- | | |
| | carboxamide | | |
| 278 | 2-[5-chloro-6-(3,3- | 418.1 | ¹H NMR (400 MHz, DMSO-d₆) δ 12.07 (d, |
| | difluoro-1-methyl- | 419.3 | J = 1.9 Hz, 1H), 8.52 (d, J = 5.8 Hz, 1H), |
| | cyclobutyl)-2- | | 8.09 (s, 1H), 7.58-7.48 (m, 1H), 7.43 (d, J = |
| | methyl-3-pyridyl]-4- | | 5.8 Hz, 1H), 7.32 (s, 1H), 6.24 (d, J = 1.6 |
| | oxo-1H-1,6- | | Hz, 1H), 3.32 (s, 3H), 3.29-3.15 (m, 2H), |
| | naphthyridine-5- | | 2.90-2.77 (m, 2H), 1.59 (s, 3H). |
| | carboxamide | | |
| 279 | 2-[5-chloro-6-(3,3- | 432.12 | ¹H NMR (400 MHz, CD₃OD) δ 8.56 (d, J = |
| | difluoro-1-methyl- | 433.3 | 5.9 Hz, 1H), 7.92 (s, 1H), 7.53 (d, J = 5.9 |
| | cyclopentyl)-2- | | Hz, 1H), 6.37 (s, 1H), 3.10-2.94 (m, 1H), |
| | methyl-3-pyridyl]-4- | | 2.80-2.67 (m, 1H), 2.54 (s, 3H), 2.52-2.41 |
| | oxo-1H-1,6- | | (m, 1H), 2.40-2.10 (m, 3H), 1.57 (s, 3H). |
| | naphthyridine-5- | | |
| | carboxamide | | |
| 280 | 2-(5-chloro-2- | 408.14 | ¹H NMR (400 MHz, DMSO-d₆) δ 12.53 (br |
| | methyl-6- | 409.3 | s, 1H), 8.57 (d, J = 6.0 Hz, 1H), 7.99 (s, |
| | spiro[3.3]heptan-2- | | 1H), 7.82 (br s, 1H), 7.64 (d, J = 6.0 Hz, |
| | yl-3-pyridyl)-4-oxo- | | 2H), 6.33 (s, 1H), 3.82 (p, J = 8.6 Hz, 1H), |
| | 1H-1,6- | | 2.51 (s, 3H), 2.43-2.30 (m, 4H), 2.15 (app |
| | naphthyridine-5- | | t, J = 7.3 Hz, 2H), 1.96-1.88 (m, 2H), 1.88- |
| | carboxamide | | 1.76 (m, 2H). |
| 281 | 2-[5-chloro-2- | 424.09 | ¹H NMR (400 MHz, DMSO-d₆) δ 12.08 (s, |
| | methyl-6-(2,2,2- | 425.4 | 1H), 8.52 (d, J = 5.8 Hz, 1H), 8.12 (s, 1H), |
| | trifluoro-1,1- | | 7.53 (s, 1H), 7.43 (d, J = 5.8 Hz, 1H), 7.36- |
| | dimethyl-ethyl)-3- | | 7.26 (m, 1H), 6.27 (s, 1H), 2.5 (s, 3H), 1.79 |
| | pyridyl]-4-oxo-1H- | | (s, 6H). |
| | 1,6-naphthyridine-5- | | |
| | carboxamide | | |
| 282 | 2-[5-chloro-2- | 382.12 | ¹H-NMR (400 MHz, DMSO-d₆) δ 12.05 (s, |
| | methyl-6-(1- | 383.2 | 1H), 8.52 (d, J = 5.7 Hz, 1H), 7.96 (s, 1H), |
| | methylcyclobutyl)-3- | | 7.53 (s, 1H), 7.43 (d, J = 5.6 Hz, 1H), 7.32 |
| | pyridyl]-4-oxo-1H- | | (s, 1H), 6.23 (s, 1H), 2.68-2.55 (m, 2H), |
| | 1,6-naphthyridine-5- | | 2.48 (s, 3H), 2.16-2.08 (m, 3H), 1.78-1.71 |
| | carboxamide | | (m, 1H), 1.56 (s, 3H) |
| 283 | 2-[5-chloro-2- | 394.12 | ¹H NMR (400 MHz, CD₃OD) δ 8.56 (d, J = |
| | methyl-6-(3-methyl- | 395.2 | 5.9 Hz, 1H), 7.82 (s, 1H), 7.59-7.46 (m, |
| | 1- | | 1H), 6.35 (br s, 1H), 2.51 (s, 3H), 2.20 (s, |
| | bicyclo[1.1.1]pentanyl)- | | 6H), 1.27 (s, 3H). |
| | 3-pyridyl]-4-oxo- | | |
| | 1H-1,6- | | |
| | naphthyridine-5- | | |
| | carboxamide | | |
| 284 | 2-(5-chloro-2- | 408.14 | ¹H NMR (400 MHz, DMSO-d₆) δ 12.73 (br |
| | methyl-6-norbornan- | 409.3 | s, 1H), 8.60 (d, J = 6.1 Hz, 1H), 7.98 (s, |
| | 1-yl-3-pyridyl)-4- | | 1H), 7.92 (br s, 1H), 7.77 (br s, 1H), 7.72 (d, |
| | oxo-1H-1,6- | | J = 6.0 Hz, 1H), 6.40 (s, 1H), 2.49 (s, 3H), |
| | naphthyridine-5- | | 2.32-2.28 (m, 1H), 2.15-2.05 (m, 2H), |
| | carboxamide | | 1.93 (s, 2H), 1.83-1.69 (m, 4H), 1.50-1.41 |
| | | | (m, 2H). |
| 285 | 2-[5-chloro-2- | 396.14 | ¹H NMR (400 MHz, DMSO-d₆) δ 12.03 (s, |
| | methyl-6-(1- | 397.3 | 1H), 8.51 (d, J = 5.8 Hz, 1H), 7.99 (s, 1H), |
| | methylcyclopentyl)- | | 7.52 (s, 1H), 7.42 (d, J = 5.8 Hz, 1H), 7.32 |
| | 3-pyridyl]-4-oxo-1H- | | (s, 1H), 6.24 (d, J = 1.6 Hz, 1H), 2.48 (s, |
| | 1,6-naphthyridine-5- | | 3H), 2.38-2.27 (m, 2H), 1.96-1.86 (m, |
| | carboxamide | | 2H), 1.80-1.69 (m, 2H), 1.69-1.57 (m, |
| | | | 2H), 1.41 (s, 3H). |
| 286 | 2-[5-chloro-2- | 368.1 | ¹H-NMR (400 MHz, DMSO-d₆) δ 12.01 (s, |
| | methyl-6-(1- | 369.19 | 1H), 8.48 (d, J = 5.3 Hz, 1H), 7.97 (s, 1H), |
| | methylcyclopropyl)- | | 7.48 (s, 1H), 7.38 (d, J = 5.3 Hz, 1H), 7.28 |
| | 3-pyridyl]-4-oxo-1H- | | (s, 1H), 6.18 (s, 1H), 2.43 (s, 3H), 1.38 (s, |
| | 1,6-naphthyridine-5- | | 3H), 0.93-0.88 (m, 2H), 0.82-0.78 (m, 2H) |
| | carboxamide | | |
| 287 | 2-[5-chloro-6-(2,2- | 411.15 | ¹H NMR (400 MHz, CD₃OD) δ 8.52 (d, J = |
| | dimethylpyrrolidin- | 412.1 | 5.9 Hz, 1H), 7.60 (s, 1H), 7.53 (d, J = 5.9 |
| | 1-yl)-2-methyl-3- | | Hz, 1H), 6.32 (s, 1H), 3.96 (t, J = 6.8 Hz, |
| | pyridyl]-4-oxo-1H- | | 2H), 2.42 (s, 3H), 1.96 (p, J = 6.9 Hz, 2H), |
| | 1,6-naphthyridine-5- | | 1.86 (q, J = 7.2 Hz, 2H), 1.62 (s, 6H). |
| | carboxamide | | |
| 288 | 2-(4-tert-butyl-3,5- | 371.14 | ¹H NMR (400 MHz, DMSO-d₆) δ 12.01 (s, |
| | difluoro-2-methyl- | 372.5 | 1H), 8.51 (d, J = 5.7 Hz, 1H), 7.51 (s, 1H), |
| | phenyl)-4-oxo-1H- | | 7.43 (d, J = 5.8 Hz, 1H), 7.31 (s, 1H), 7.22 |
| | 1,6-naphthyridine-5- | | (d, J = 13.0 Hz, 1H), 6.14 (s, 1H), 2.15 (d, |
| | carboxamide | | J = 3.2 Hz, 3H), 1.52-1.43 (m, 9H). |
| 289 | 2-(4-tert-butyl-5- | 387.11 | ¹H NMR (400 MHz, CD₃OD) δ 8.51 (d, J = |
| | chloro-3-fluoro-2- | 388.1 | 5.8 Hz, 1H), 7.53 (d, J = 5.9 Hz, 1H), 7.33 |
| | methyl-phenyl)-4- | | (s, 1H), 6.31 (s, 1H), 2.20 (d, J = 3.7 Hz, |
| | oxo-1H-1,6- | | 3H), 1.62 (d, J = 3.4 Hz, 9H). |
| | naphthyridine-5- | | |
| | carboxamide | | |
| 290 | 2-(4-tert-butyl-3- | 387.11 | ¹H NMR (400 MHz, DMSO-d₆) δ 12.14 (s, |
| | chloro-2-fluoro-6- | 388.75 | 1H), 8.51 (d, J = 5.7 Hz, 1H), 7.55 (s, 1H), |
| | methyl-phenyl)-4- | | 7.40 (d, J = 5.8 Hz, 1H), 7.37 (d, J = 1.6 Hz, |
| | oxo-1H-1,6- | | 1H), 7.32 (s, 1H), 6.20 (s, 1H), 2.27 (s, 3H), |
| | naphthyridine-5- | | 1.50 (s, 9H). |
| | carboxamide | | |
| 291 | 2-(4-tert-butyl-5- | 353.15 | ¹H NMR (400 MHz, CD₃OD) δ 8.50 (d, J = |
| | fluoro-2-methyl- | — | 5.9 Hz, 1H), 7.53 (d, J = 5.9 Hz, 1H), 7.33 |
| | phenyl)-4-oxo-1H- | | (d, J = 8.2 Hz, 1H), 7.13 (d, J = 12.7 Hz, |
| | 1,6-naphthyridine-5- | | 1H), 6.31 (s, 1H), 2.31 (s, 3H), 1.44-1.39 |
| | carboxamide | | (m, 9H). |
| 292 | 2-(4-tert-butyl-2,3- | 371.14 | ¹H NMR (400 MHz, CD₃OD) δ 8.74 (s, 1H), |
| | difluoro-6-methyl- | 372.2 | 7.78 (s, 1H), 7.22 (d, J = 7.0 Hz, 1H), 6.72 |
| | phenyl)-4-oxo-1H- | | (s, 1H), 2.30 (s, 3H), 1.44 (s, 9H). |
| | 1,6-naphthyridine-5- | | |
| | carboxamide | | |
| 293 | methyl 5-tert-butyl- | 413.11 | ¹H NMR (400 MHz, CDCl₃) δ 15.49 (s, 1H), |
| | 2-(5-carbamoyl-4- | 414.1 | 8.98 (br. s, 1H), 8.64 (d, J = 5.6 Hz, 1H), |
| | oxo-1H-1,6- | | 8.10 (d, J = 5.4 Hz, 1H), 7.95 (s, 1H), 7.68 |
| | naphthyridin-2-yl)-4- | | (s, 1H), 7.23 (s, 1H), 6.16 (br. s, 1H), 3.68 |
| | chloro-benzoate | | (s, 3H), 1.56 (s, 9H). |
| 294 | 2-[4-tert-butyl-5- | 385.12 | ¹H NMR (400 MHz, DMSO-d₆) δ 11.97 (br. |
| | chloro-2- | 386.1 | s, 1H), 8.51 (d, J = 4.9 Hz, 1H), 7.77 (s, |
| | (hydroxymethyl) | | 1H), 7.58-7.49 (m, 2H), 7.45 (d, J = 5.6 |
| | phenyl]-4-oxo-1H-1,6- | | Hz, 1H), 7.31 (br. s, 1H), 6.19 (s, 1H), 5.41 |
| | naphthyridine-5- | | (br. s, 1H), 4.52 (s, 2H), 1.51 (s, 9H). |
| | carboxamide | | |
| 295 | 2-[4-(2-hydroxy-1,1- | 419.15 | ¹H NMR (400 MHz, DMSO-d₆) δ 12.03 (s, |
| | dimethyl-ethyl)-2- | 420.2 | 1H), 8.50 (d, J = 5.9 Hz, 1H), 7.76 (s, 1H), |
| | methyl-5- | | 7.73 (s, 1H), 7.53 (br. s, 1H), 7.43 (d, J = 5.9 |
| | (trifluoromethyl) | | Hz, 1H), 7.32 (br. s, 1H), 6.16 (s, 1H), 4.92 |
| | phenyl]-4-oxo-1H-1,6- | | (br. s, 1H), 3.65 (s, 2H), 2.38 (s, 3H), 1.38 |
| | naphthyridine-5- | | (s, 6H). |
| | carboxamide | | |
| 296 | 2-(4-tert-butyl-5- | 342.11 | |
| | chloro-2-methyl- | | |
| | phenyl)-6-oxido-1H- | | |
| | 1,6-naphthyridin-6- | | |
| | ium-4-one | | |
| 297 | 2-[5-chloro-6-(2- | 386.11 | 1H NMR (400 MHz, DMSO-d6) δ 12.06 |
| | hydroxy-1,1- | 387 | (br. s., 1H), 8.51 (d, J = 5.6 Hz, 1H), 7.95 (s, |
| | dimethyl-ethyl)-2- | | 1H), 7.52 (br. s., 1H), 7.42 (d, J = 5.4 Hz, |
| | methyl-3-pyridyl]-4- | | 1H), 7.31 (br. s., 1H), 6.22 (s, 1H), 4.61 (t, |
| | oxo-1H-1,6- | | J = 5.7 Hz, 1H), 3.84 (d, J = 5.9 Hz, 2H), 2.48 |
| | naphthyridine-5- | | (s, 3H), 1.43 (s, 6H). |
| | carboxamide | | |
| 298 | 2-[6-(1- | 362.09 | 1H NMR (400 MHz, CD3OD) δ 8.72 (d, J = |
| | bicyclo[1.1.1] | 363.3 | 5.9 Hz, 1H), 7.86 (s, 1H), 7.68 (d, J = 5.9 |
| | pentanyl)-5-chloro-2- | | Hz, 1H), 6.40 (s, 1H), 2.57 (s, 1H), 2.52 (s, |
| | methyl-3-pyridyl]-4- | | 3H), 2.37 (s, 6H). |
| | oxo-1H-1,6- | | |
| | naphthyridine-5- | | |
| | carbonitrile | | |
| 299 | 2-(5-chloro-2- | 390.12 | 1H NMR (400 MHz, CD3OD) δ 8.73 (d, J = |
| | methyl-6- | 391.3 | 5.9 Hz, 1H), 7.94 (s, 1H), 7.70 (d, J = 5.9 |
| | spiro[3.3]heptan-2- | | Hz, 1H), 6.43 (s, 1H), 3.91 (p, J = 8.7 Hz, |
| | yl-3-pyridyl)-4-oxo- | | 1H), 2.58 (s, 3H), 2.48-2.37 (m, 4H), 2.20 |
| | 1H-1,6- | | (app t, J = 7.3 Hz, 2H), 2.00-1.93 (m, 2H), |
| | naphthyridine-5- | | 1.93-1.83 (m, 2H). |
| | carbonitrile | | |
| 300 | 2-(5-chloro-2- | 314.06 | 1H NMR (400 MHz, DMSO-d6) δ 12.09 (s, |
| | methyl-3-pyridyl)-4- | 315.2 | 1H), 8.70 (d, J = 2.5 Hz, 1H), 8.52 (d, J = |
| | oxo-1H-1,6- | | 5.8 Hz, 1H), 8.10 (d, J = 2.5 Hz, 1H), 7.53 |
| | naphthyridine-5- | | (s, 1H), 7.44 (d, J = 5.8 Hz, 1H), 7.32 (s, |
| | carboxamide | | 1H), 6.23 (d, J = 1.6 Hz, 1H), 3.29 (s, 3H). |
| 301 | 2-(5-chloro-2- | 390.12 | 1H NMR (400 MHz, CD3OD) δ 8.70 (d, J = |
| | methyl-6-norbornan- | 391.3 | 5.8 Hz, 1H), 7.83 (s, 1H), 7.69 (d, J = 5.8 |
| | 1-yl-3-pyridyl)-4- | | Hz, 1H), 6.42 (s, 1H), 2.51 (s, 3H), 2.34- |
| | oxo-1H-1,6- | | 2.30 (m, 1H), 2.22-2.14 (m, 2H), 1.99 (s, |
| | naphthyridine-5- | | 2H), 1.86-1.74 (m, 4H), 1.55-1.46 (m, |
| | carbonitrile | | 2H). |
| 302 | 2-(5-chloro-2- | 408.14 | 1H NMR (400 MHz, CD3OD) δ 8.89 (d, J = |
| | methyl-6-norbornan- | 409.29 | 6.1 Hz, 1H), 8.03 (d, J = 6.0 Hz, 1H), 7.98 |
| | 2-yl-3-pyridyl)-4- | | (s, 1H), 7.05 (s, 1H), 3.30-3.27 (m, 1H), |
| | oxo-1H-1,6- | | 2.58 (s, 3H), 2.43-2.36 (m, 2H), 2.36-2.26 |
| | naphthyridine-5- | | (m, 1H), 1.74 (dt, J = 9.6, 2.0 Hz, 1H), 1.70- |
| | carboxamide | | 1.58 (m, 3H), 1.54-1.45 (m, 1H), 1.45- |
| | | | 1.32 (m, 1H), 1.19-1.12 (m, 1H). |

### Example 5

### 2-(4-tert-butyl-5-chloro-2-methyl-phenyl)-4-oxo-1H-1,6-naphthyridine-5-carboxamide (303)

A microwave reaction vial charged with 2-chloro-4-[(4-methoxyphenyl)methoxy]-1,6-naphthyridine-5-carbonitrile (100 mg, 0.31 mmol), 2-(4-tert-butyl-5-chloro-2-methyl-phenyl)-4,4,5,5-tetramethyl-1,3,2-dioxaborolane (130 mg, 0.32 mmol), SPhos Pd G3 (36 mg, 0.05 mmol), potassium phosphate (170 mg, 0.80 mmol), dioxane (1.5 mL) and water (150 μL) was flushed with nitrogen for 60 seconds, capped and heated for 1 hour at 50° C. The reaction mixture was diluted with water and the aqueous layer was extracted with DCM (3×). The combined organic layers were dried over magnesium sulfate, filtered and concentrated. The PMB-protected intermediate was taken up in toluene (2 mL) and TFA (2 mL) and stirred at 60° C. for 6 hours. The solvent was evaporated and the crude material purified by silica gel chromatography using 0-100% ethyl acetate in DCM, followed by a second purification by reverse phase chromatography using 0-40% acetonitrile in water (5 mM HCl) to obtain 2-(4-tert-butyl-5-chloro-2-methyl-phenyl)-4-oxo-1H-1,6-naphthyridine-5-carboxamide (303, 32 mg, 28%). ESI-MS m/z calc. 369.12, found 370.4 (M+1)⁺. ¹H NMR (400 MHz, DMSO-d₆) δ (ppm) 12.04 (s, 1H), 8.56 (d, J=5.8 Hz, 1H), 7.58 (s, 1H), 7.55 (s, 2H), 7.50 (d, J=5.8 Hz, 1H), 7.37 (s, 1H), 6.19 (s, 1H), 2.37 (s, 3H), 1.55 (s, 9H).

### Example 6

### 2-(4-tert-butyl-2-methyl-phenyl)-6-[2-(dimethylamino)ethoxy]-1H-1,5-naphthyridin-4-one (304)

### Step 1: 8-benzyloxy-6-(4-tert-butyl-2-methyl-phenyl)-1-oxido-1,5-naphthyridin-1-ium

A mixture of 8-benzyloxy-6-chloro-1-oxido-1,5-naphthyridin-1-ium (400 mg, 1.39 mmol) ( ), 2-(4-tert-butyl-2-methyl-phenyl)-4,4,5,5-tetramethyl-1,3,2-dioxaborolane (432 mg, 1.57 mmol), potassium carbonate (450 mg, 3.26 mmol), X-Phos (33 mg, 0.07 mmol), XPhos Pd G2 (123 mg, 0.08 mmol), dioxane (5 mL) and water (500 μL) in a microwave vial was purged with nitrogen for 30 seconds, capped and stirred at 60° C. for 16 h. The crude was diluted with water and ethyl acetate. The layers were separated, and the aqueous layer was extracted with ethyl acetate (2×). The combined organic layers were dried over sodium sulfate, filtered, concentrated, and purified by silica gel column chromatography using a gradient of ethyl acetate and hexane to obtain 8-benzyloxy-6-(4-tert-butyl-2-methyl-phenyl)-1-oxido-1,5-naphthyridin-1-ium (309 mg, 54%). ESI-MS m/z calc. 398.19, found 399.3 (M+1)⁺.

### Step 2: 4-benzyloxy-2-(4-tert-butyl-2-methyl-phenyl)-6-chloro-1,5-naphthyridine

8-benzyloxy-6-(4-tert-butyl-2-methyl-phenyl)-1-oxido-1,5-naphthyridin-1-ium (224 mg, 0.56 mmol) was taken up in POCl₃ (500 μL, 5.36 mmol) and was stirred at 50° C. for 30 min. The reaction mixture was concentrated under reduced pressure (no heat). The crude was suspended in DCM and cooled down using a dry-ice/acetone bath and quenched slowly with ice. The layers were separated, and the organic layer was dried over sodium sulfate, filtered and concentrated. Purification by reverse phase HPLC (C₁₈) using 30 to 99% acetonitrile in water (HCl modifier) provided 4-benzyloxy-2-(4-tert-butyl-2-methyl-phenyl)-6-chloro-1,5-naphthyridine (133.5 mg, 56%). ESI-MS m/z calc. 416.16, found 417.3 (M+1)⁺.

### Step 3: 2-(4-tert-butyl-2-methyl-phenyl)-6-[2-(dimethylamino)ethoxy]-1H-1,5-naphthyridin-4-one (304)

Sodium hydride (11.7 mg of 60% w/w, 0.29 mmol) was added to a solution of 2-(dimethylamino)ethanol (15 mg, 0.17 mmol) in DMF (0.5 mL) at 0° C. and stirred for 1 h. 4-benzyloxy-2-(4-tert-butyl-2-methyl-phenyl)-6-chloro-1,5-naphthyridine (13 mg, 0.03 mmol) as a solution in DMF (0.5 mL) was slowly added and the resulting reaction mixture was stirred at 100° C. for 2 h. The reaction mixture was filtered, and purified by reverse phase preparative column chromatography (C₁₈) using 30 to 99% acetonitrile in water (HCl modifier) to obtain 2-[[8-benzyloxy-6-(4-tert-butyl-2-methyl-phenyl)-1,5-naphthyridin-2-yl]oxy]-N,N-dimethyl-ethanamine (5.7 mg, 38%). ESI-MS m/z calc. 469.27, found 470.0 (M+1)⁺.

Palladium on carbon (5 mg of 10% w/w, 0.005 mmol) was added to a solution of 2-[[8-benzyloxy-6-(4-tert-butyl-2-methyl-phenyl)-1,5-naphthyridin-2-yl]oxy]-N,N-dimethyl-ethanamine (5.7 mg) in methanol (2 mL). The reaction mixture was purged with nitrogen for 30 see and the reaction mixture was stirred at room temperature under a balloon of hydrogen for 1 h. The crude was filtered, and purified by reverse phase preparative column chromatography (C₁₈) using 10 to 50% acetonitrile in water (HCl modifier) to obtain 2-(4-tert-butyl-2-methyl-phenyl)-6-[2-(dimethylamino)ethoxy]-1H-1,5-naphthyridin-4-one (304, 3.8 mg, 32%). ESI-MS m/z calc. 379.23, found 380.3 (M+1)⁺.

### Example 7

### 2-(4-tert-butyl-2-methyl-phenyl)-6-[2-methoxyethyl(methyl)amino]-1H-1,5-naphthyridin-4-one (305)

### Step 1: 2-(4-tert-butyl-2-methyl-phenyl)-6-[2-methoxyethyl(methyl)amino]-1H-1,5-naphthyridin-4-one (305)

To a mixture of 4-benzyloxy-2-(4-tert-butyl-2-methyl-phenyl)-6-chloro-1,5-naphthyridine (15 mg, 0.04 mmol) ( ) and 2-methoxy-N-methyl-ethanamine (6 mg, 0.07 mmol) in DMF (500 μL) was added cesium carbonate (35 mg, 0.11 mmol). The reaction mixture was stirred at 80° C. for 16 h. The reaction mixture was cooled to room temperature and filtered. Purification by reverse phase preparative column chromatography (C₁₈) using of 20 to 70% acetonitrile in water (HCl modifier) gave 8-benzyloxy-6-(4-tert-butyl-2-methyl-phenyl)-N-(2-methoxyethyl)-N-methyl-1,5-naphthyridin-2-amine (9 mg, 43%) ESI-MS m/z calc. 469.27, found 470.0 (M+1)⁺.

Palladium on carbon (5 mg of 10% w/w, 0.005 mmol) was added to a solution of 8-benzyloxy-6-(4-tert-butyl-2-methyl-phenyl)-N-(2-methoxyethyl)-N-methyl-1,5-naphthyridin-2-amine (4.5 mg) in methanol (2 mL). The reaction mixture was purged with nitrogen for 30 seconds and stirred at room temperature for 45 minutes under a balloon of hydrogen. The reaction mixture was filtered, concentrated and purified by reverse phase HPLC using 15 to 60% acetonitrile in water (HCl modifier) to obtain 2-(4-tert-butyl-2-methyl-phenyl)-6-[2-methoxyethyl(methyl)amino]-1H-1,5-naphthyridin-4-one (305, 4.5 mg, 33%). ESI-MS m/z calc. 379.23, found 380.3 (M+1)⁺.

### Example 8

### 2-(4-tert-butyl-2-methyl-phenyl)-5-[2-(dimethylamino)ethoxy]-1H-1,6-naphthyridin-4-one (306) and 2-(4-tert-butyl-2-methyl-phenyl)-1,6-naphthyridine-4,5-diol (307)

### Step 1: 24-benzyloxy-2-(4-tert-butyl-2-methyl-phenyl)-6-oxido-1,6-naphthyridin-6-ium

A microwave vial charged with 4-benzyloxy-2-chloro-6-oxido-1,6-naphthyridin-6-ium (750 mg, 2.62 mmol)), 2-(4-tert-butyl-2-methyl-phenyl)-4,4,5,5-tetramethyl-1,3,2-dioxaborolane (715 mg, 2.6 mmol), potassium phosphate (1.53 g, 7.2 mmol), SPhos Pd G3 (406 mg, 0.52 mmol), dioxane (10 mL) and water (1 mL) was degassed using nitrogen for 1 minute. The microwave vial was sealed and heated at 70° C. for 18 hours. The reaction mixture was diluted with ethyl acetate (5 ml) and water (5 ml). The layers were separated, and the aqueous layer was extracted with ethyl acetate (2×). The combined organic layers were dried over sodium sulfate, filtered and concentrated. Purification by silica gel column chromatography using ethyl acetate and hexane gave 4-benzyloxy-2-(4-tert-butyl-2-methyl-phenyl)-6-oxido-1,6-naphthyridin-6-ium (451 mg, 43%). ESI-MS m/z calc. 398.19, found 399.3 (M+1)⁺.

### Step 2: 4-benzyloxy-2-(4-tert-butyl-2-methyl-phenyl)-5-chloro-1,6-naphthyridine

A vial charged with 4-benzyloxy-2-(4-tert-butyl-2-methyl-phenyl)-6-oxido-1,6-naphthyridin-6-ium (90 mg, 0.22 mmol) and POCl₃ (210 μL, 2.25 mmol) was heated at 50° C. for 30 minutes. The reaction mixture was poured onto ice water and quenched slowly with saturated sodium bicarbonate solution. The aqueous layer was extracted with ethyl acetate, dried over magnesium sulfate, filtered and concentrated. The crude material was purified via silica gel column chromatography using 0 to 100% ethyl acetate in hexanes to obtain 4-benzyloxy-2-(4-tert-butyl-2-methyl-phenyl)-5-chloro-1,6-naphthyridine (45 mg, 43%). ESI-MS m/z calc. 416.16, found 417.5 (M+1)⁺. ¹H NMR (400 MHz, DMSO-d₆) δ (ppm) 8.46 (d, J=5.7 Hz, 1H), 7.80 (d, J=5.7 Hz, 1H), 7.64-7.57 (m, 2H), 7.50-7.41 (m, 4H), 7.40-7.29 (m, 3H), 5.54 (s, 2H), 2.35 (s, 3H), 1.33 (s, 9H).

### Step 3: 2-(4-tert-butyl-2-methyl-phenyl)-5-[2-(dimethylamino)ethoxy]-1H-1,6-naphthyridin-4-one (306) and 2-(4-tert-butyl-2-methyl-phenyl)-1,6-naphthyridine-4,5-diol (307)

Sodium hydride (7 mg of 60% w/w, 0.1750 mmol) was added to N,N-Dimethylaminoethanol (13.6 mg, 0.15 mmol) in DMF (500 μL) at 0° C. The reaction mixture was stirred at 0° C. for 1 h. 4-benzyloxy-2-(4-tert-butyl-2-methyl-phenyl)-5-chloro-1,6-naphthyridine (25 mg, 0.05 mmol) in DMF (500 μL) was added and the reaction mixture was stirred at 100° C. for 2 h. The reaction mixture was cooled to room temperature, filtered, and purified by reverse phase preparative chromatography (C₁₈) using 15 to 50% acetonitrile in water (HCl modifier) to obtain 2-(4-tert-butyl-2-methyl-phenyl)-5-[2-(dimethylamino)ethoxy]-1H-1,6-naphthyridin-4-one (306, approximately 9.1 mg, 38%) ESI-MS m/z calc. 379.23, found 380.0 (M+1)⁺. 2-(4-tert-butyl-2-methyl-phenyl)-1,6-naphthyridine-4,5-diol (307, 2.9 mg, 16%) was also isolated. ESI-MS m/z calc. 308.15, found 309.1 (M+1)⁺.

### Example 9

### methyl 2-(4-tert-butyl-2-methyl-phenyl)-4-oxo-1H-1,6-naphthyridine-5-carboxylate (308)

### Step 1: methyl 4-benzyloxy-2-(4-tert-butyl-2-methyl-phenyl)-1,6-naphthyridine-5-carboxylate

A methanol (2 mL) mixture of 4-benzyloxy-2-(4-tert-butyl-2-methyl-phenyl)-5-chloro-1,6-naphthyridine (30 mg, 0.06 mmol), Pd(dppf)Cl₂·DCM (12 mg, 0.01 mmol), and DIPEA (70 μL, 0.40 mmol) was directly added to a stainless-steel high-pressure reaction vessel and the system was evacuated under vacuum and pressurized with carbon monoxide gas. This process was repeated twice. The sealed vessel was heated to 90° C. at 100 PSI carbon monoxide for 4 h. The reaction was filtered over a celite bed and concentrated. The crude material was purified via silica gel column chromatography using 0 to 30% ethyl acetate in hexanes to obtain methyl 4-benzyloxy-2-(4-tert-butyl-2-methyl-phenyl)-1,6-naphthyridine-5-carboxylate (25 mg, 88%). ESI-MS m/z calc. 440.21, found 441.5 (M+1)⁺.

### Step 2: methyl 2-(4-tert-butyl-2-methyl-phenyl)-4-oxo-1H-1,6-naphthyridine-5-carboxylate (308)

To a solution of methyl 4-benzyloxy-2-(4-tert-butyl-2-methyl-phenyl)-1,6-naphthyridine-5-carboxylate (15 mg, 0.03405 mmol) in methanol (600 μL) was added Pd/C (6 mg of 10% w/w, 0.005 mmol) and the reaction mixture was evacuated under vacuum and back filled with nitrogen. The reaction mixture was again evacuated under vacuum and a balloon filled with hydrogen was placed on it. After 10 minutes, the reaction mixture was filtered through a plug of celite and washed with methanol. The solvent was evaporated, and the crude material was purified via silica gel column chromatography using 0 to 100% ethyl acetate in hexanes to obtain methyl 2-(4-tert-butyl-2-methyl-phenyl)-4-oxo-1H-1,6-naphthyridine-5-carboxylate (308, 6.7 mg, 56%). ESI-MS m/z calc. 350.16, found 351.54 (M+1)⁺. ¹H NMR (400 MHz, DMSO-d₆) δ (ppm) 12.21 (s, 1H), 8.56 (d, J=5.8 Hz, 1H), 7.54 (d, J=5.8 Hz, 1H), 7.47-7.41 (m, 1H), 7.41-7.35 (m, 2H), 6.12 (s, 1H), 3.86 (s, 3H), 2.32 (s, 3H), 1.32 (s, 9H).

### Example 10

### 2-(1,1,6-trimethylindan-5-yl)-1,6-naphthyridine-4,5-diol (309)

### Step 1: 4-benzyloxy-6-oxido-2-(1,1,6-trimethylindan-5-yl)-1,6-naphthyridin-6-ium

In a microwave reaction vial, 4-benzyloxy-2-chloro-6-oxido-1,6-naphthyridin-6-ium (150 mg, 0.52 mmol) ( ), was dissolved in DMSO (2.6 mL) along with 4,4,5,5-tetramethyl-2-(1,1,6-trimethylindan-5-yl)-1,3,2-dioxaborolane (170 mg, 0.59 mmol) and potassium carbonate (765 μL of 2 M, 1.53 mmol). The reaction was flushed with nitrogen then Pd(dppf)Cl₂ DCM (41 mg, 0.05 mmol) was added. The reaction was flushed once more with nitrogen, capped, subjected to microwave irradiation for 1 h. at 120° C. The reaction mixture was diluted with ethyl acetate and filtered. The filtrate was washed with water followed by saturated sodium chloride solution. The organic layer was separated, dried over sodium sulfate, filtered, and evaporated to dryness. The crude material was purified by silica gel column chromatography using 0 to 15% methanol in DCM gradient to obtain 4-benzyloxy-6-oxido-2-(1,1,6-trimethylindan-5-yl)-1,6-naphthyridin-6-ium (96 mg, 45%). ESI-MS m/z calc. 410.19, found 411.4 (M+1)⁺. ¹H NMR (400 MHz, CDCl₃) δ (ppm) 9.11 (d, J=2.0 Hz, 1H), 8.31 (dd, J=7.3, 2.1 Hz, 1H), 7.86 (d, J=7.3 Hz, 1H), 7.47-7.37 (m, 5H), 7.26 (s, 1H), 7.05 (d, J=13.0 Hz, 2H), 5.31 (s, 2H), 2.91 (t, J=7.2 Hz, 2H), 2.29 (s, 3H), 1.96 (t, J=7.2 Hz, 2H), 1.29 (s, 6H).

### Step 2: 2-(1,1,6-trimethylindan-5-yl)-1,6-naphthyridine-4,5-diol (309)

In a reaction vial, 4-benzyloxy-6-oxido-2-(1,1,6-trimethylindan-5-yl)-1,6-naphthyridin-6-ium (96 mg, 0.23 mmol) was mixed with acetyl acetate (936 μL, 9.92 mmol) and heated at 130° C. for 3 h. The reaction mixture was cooled to 100° C. and water was added. The reaction mixture was stirred at 100° C. for 30 min and cooled to room temperature. The aqueous layer was extracted with DCM and the organic layer was washed with saturated sodium bicarbonate/saturated sodium carbonate solutions (3×). The organic layer was, dried over magnesium sulfate, filtered, and evaporated to dryness. The crude material was then dissolved in ethanol (2 mL) and purged with nitrogen. To the reaction mixture, Pd/C (62 mg of 10% w/w, 0.058 mmol) was added and the reaction was purged with hydrogen. The reaction mixture was stirred under an atmosphere of hydrogen using a balloon for 2 h. and filtered through celite. The filtrate was evaporated to dryness and the crude material was purified by preparative HPLC (C₁₈) using 10 to 99% ACN in water (HCl modifier) to obtain 2-(1,1,6-trimethylindan-5-yl)-1,6-naphthyridine-4,5-diol (309, 23.7 mg, 32%). ESI-MS m/z calc. 320.15, found 321.3 (M+1)⁺. ¹H NMR (400 MHz, CD₃OD) δ (ppm) 7.88 (d, J=7.5 Hz, 1H), 7.33 (s, 1H), 7.25 (s, 1H), 7.20 (s, 1H), 6.83 (d, J=7.4 Hz, 1H), 2.95 (t, J=7.2 Hz, 2H), 2.37 (s, 3H), 2.00 (t, J=7.2 Hz, 2H), 1.30 (s, 6H).

### Example 11

### 2-(4-tert-butyl-2-methyl-phenyl)-5-methoxy-1H-1,6-naphthyridin-4-one (310)

### Step 1: 2-(4-tert-butyl-2-methyl-phenyl)-5-methoxy-1H-1,6-naphthyridin-4-one

To a solution of 4-benzyloxy-2-4-tert-butyl-2-methyl-phenyl-5-chloro-1,6-napthyridine (25 mg, 0.06 mmol) ( ) in methanol (5 mL) was added potassium carbonate (30 mg, 0.22 mmol) and the reaction mixture was stirred at 100° C. for 16 h. The crude was filtered, concentrated, and purified by reverse phase preparative column chromatography (C₁₈) using a gradient of 20 to 70% acetonitrile in water (HCl modifier) to obtain 4-benzyloxy-2-(4-tert-butyl-2-methyl-phenyl)-5-methoxy-1,6-naphthyridine (7.0 mg, 27%). ESI-MS m/z calc. 412.21, found 413.3 (M+1)⁺.

Pd/C (3 mg of 10% w/w, 0.003 mmol) was added to a solution of 4-benzyloxy-2-(4-tert-butyl-2-methyl-phenyl)-5-methoxy-1,6-naphthyridine (7.0 mg) in methanol (2 mL). The mixture was purged with nitrogen for 30 seconds and was stirred at room temperature under an atmosphere of hydrogen using a balloon for 90 minutes. The reaction mixture was filtered, and purified by reverse phase preparative column chromatography (C₁₈) using a gradient of 10 to 60% acetonitrile in water (HCl modifier) to give 2-(4-tert-butyl-2-methyl-phenyl)-5-methoxy-1H-1,6-naphthyridin-4-one (310, 1.6 mg, 8%). ESI-MS m/z calc. 322.17, found 323.3 (M+1)⁺.

### Example 12

### 6-(4-tert-butyl-2-methyl-phenyl)-1,5-dihydro-1,5-naphthyridine-2,8-dione (311)

### Step 1: 6-(4-tert-butyl-2-methyl-phenyl)-1,5-dihydro-1,5-naphthyridine-2,8-dione (311)

To a stirred solution of benzyl alcohol (14 mg, 0.13 mmol) in DMF (500 μL) at 0° C. under an atmosphere of nitrogen was added sodium hydride (6 mg, 0.15 mmol). The reaction mixture was stirred at 0° C. for 1 h. A solution of 4-benzyloxy-2-(4-tert-butyl-2-methyl-phenyl)-6-chloro-1,5-naphthyridine (40 mg, 0.096 mmol) in DMF (500 μL) was added slowly and the resulting mixture was stirred at 50° C. for 30 minutes. The reaction mixture was quenched with water (5 ml) and the desired product crashed out, which was filtered and purified using silica gel column chromatography using ethyl acetate in hexane to give 4,6-dibenzyloxy-2-(4-tert-butyl-2-methyl-phenyl)-1,5-naphthyridine (21.6 mg, 46%). It was taken up in methanol (5 mL) and Pd/C (6 mg of 10% w/w, 0.006) was added. The reaction mixture was purged with nitrogen for 30 seconds and was stirred at room temperature under an atmosphere of hydrogen using a balloon for 20 min. The reaction mixture was filtered, concentrated, and purified by reverse phase preparative column chromatography (C₁₈) using a gradient of 15 to 60% acetonitrile in water (HCl modifier) to obtain 6-(4-tert-butyl-2-methyl-phenyl)-1,5-dihydro-1,5-naphthyridine-2,8-dione (311, 1.5 mg, 5%). ESI-MS m/z calc. 308.15, found 309.2 (M+1)⁺.

### Example 13

### 6-(4-tert-butyl-2,5-dimethyl-phenyl)-1,5-dihydro-1,5-naphthyridine-2,8-dione (312) and 2-(4-tert-butyl-2,5-dimethyl-phenyl)-6-methoxy-1H-1,5-naphthyridin-4-one (313)

### Step 1: 2-(4-tert-butyl-2,5-dimethyl-phenyl)-6-methoxy-1H-1,5-naphthyridin-4-one (313) and 6-(4-tert-butyl-2,5-dimethyl-phenyl)-1,5-dihydro-1,5-naphthyridine-2,8-dione (312)

A mixture of 4-benzyloxy-2-chloro-6-methoxy-1,5-naphthyridine (60 mg, 0.2 mmol), 2-(4-tert-butyl-2,5-dimethyl-phenyl)-4,4,5,5-tetramethyl-1,3,2-dioxaborolane (58 mg, 0.20 mmol), potassium carbonate (107 mg, 0.77 mmol), X-Phos (6.5 mg, 0.01 mmol), XPhos Pd G2 (16 mg, 0.01 mmol), ethanol (900 μL) and water (100 μL) in a microwave vial was purged with nitrogen for 30 seconds, capped and stirred at 100° C. for 16 h. The reaction mixture was filtered and purified by reverse phase preparative chromatography (C₁₈) using 30 to 70% acetonitrile in water containing 5 mM HCl to give 2-(4-tert-butyl-2,5-dimethyl-phenyl)-6-methoxy-1H-1,5-naphthyridin-4-one (313, 46.6 mg, 70%). ESI-MS m/z calc. 336.18, found 337.3 (M+1)⁺.

2-(4-tert-butyl-2,5-dimethyl-phenyl)-6-methoxy-1H-1,5-naphthyridin-4-one (46.6 mg) in acetonitrile (1 mL) was treated with HCl (200 μL of 37% w/v, 2.03 mmol), and the reaction mixture was stirred at 100° C. for 4 h. The reaction mixture was concentrated and redissolved in DMSO, filtered and purified by reverse phase preparative chromatography (C₁₈) using 30 to 40% acetonitrile in water (HCl modifier) to obtain 6-(4-tert-butyl-2,5-dimethyl-phenyl)-1,5-dihydro-1,5-naphthyridine-2,8-dione (312, 10.5 mg, 16%). ESI-MS m/z calc. 322.17, found 323.3 (M+1)⁺. ¹H NMR (400 MHz, DMSO-d₆) δ (ppm) 7.86 (d, J=9.8 Hz, 1H), 7.32 (s, 1H), 7.18 (s, 1H), 6.84 (d, J=9.8 Hz, 1H), 6.56 (s, 1H), 2.52 (s, 3H), 2.26 (s, 3H), 1.41 (s, 9H).

### Example 14

### 2-(4-tert-butyl-2-methyl-phenyl)-6-fluoro-3-(hydroxymethyl)-1H-quinolin-4-one (314)

### Step 1: 2-(4-tert-butyl-2-methyl-phenyl)-6-fluoro-3-(hydroxymethyl)-1H-quinolin-4-one (314)

A reaction vial equipped with a stir bar containing a solution of ethyl 4-benzyloxy-2-(4-tert-butyl-2-methyl-phenyl)-6-fluoro-quinoline-3-carboxylate (67 mg, 0.14 mmol) in THF (1.5 mL) was cooled to −40° C. LAH (290 μL of 2.0 M, 0.58 mmol) was added dropwise and the reaction was gradually warmed to room temperature and stirred for 5 h. The reaction mixture was cooled to 0° C. and quenched by the addition of water (30 μL) then 4M NaOH (30 μL) and stirred at room temperature for 15 minutes. The reaction mixture was filtered over celite and washed with ethyl acetate. The filtrate was concentrated in vacuo. The crude material was purified by silica gel flash column chromatography using 0 to 40% ethyl acetate in hexanes to afford 2-(4-tert-butyl-2-methyl-phenyl)-6-fluoro-3-(hydroxymethyl)-1H-quinolin-4-one (314, 5 mg, 10%). ESI-MS m/z calc. 339.16, found 340.6 (M+1)⁺. ¹H NMR (400 MHz, DMSO-d₆) δ (ppm) 11.83 (s, 1H), 7.79 (dd, J=9.4, 3.0 Hz, 1H), 7.64 (dd, J=9.1, 4.7 Hz, 1H), 7.57 (td, J=8.6, 3.0 Hz, 1H), 7.43 (d, J=2.0 Hz, 1H), 7.38 (dd, J=8.0, 2.0 Hz, 1H), 7.32 (d, J=8.0 Hz, 1H), 4.42 (t, J=5.3 Hz, 1H), 4.18 (dd, J=11.0, 5.7 Hz, 1H), 4.00 (dd, J=11.0, 5.0 Hz, 1H), 2.18 (s, 3H), 1.34 (s, 9H).

### Example 15

### 2-(4-tert-butyl-2-methyl-phenyl)-5-isopentyloxy-1H-1,6-naphthyridin-4-one (315)

### Step 1: 2-(4-tert-butyl-2-methyl-phenyl)-5-isopentyloxy-1H-1,6-naphthyridin-4-one (315)

To a solution of 3-methylbutan-1-ol (6 μL, 0.05 mmol) in DMF (400 μL) at 0° C. was added sodium hydride (2 mg of 60% w/w, 0.05 mmol) and the reaction mixture was stirred for 30 minutes. The ice bath was removed, and it was stirred for an additional 30 minutes at room temperature. The reaction mixture was again cooled to 0° C. and 4-benzyloxy-2-(4-tert-butyl-2-methyl-phenyl)-5-chloro-1,6-naphthyridine (20 mg, 0.05 mmol) as a solution in DMF (250 μL) was added and the reaction mixture was stirred at room temperature for 3 h. The reaction mixture was poured onto ice water and the aqueous layer was extracted with ethyl acetate. The combined organic layer was washed with brine, dried over magnesium sulfate, filtered and concentrated. The crude product was purified via silica gel column chromatography using 0 to 20% ethyl acetate in hexanes to obtain 4-benzyloxy-2-(4-tert-butyl-2-methyl-phenyl)-5-isopentyloxy-1,6-naphthyridine (8 mg, 36%). ESI-MS m/z calc. 468.28, found 469.8 (M+1)⁺.

It was taken up in methanol (500 μL) and Pd/C (3 mg, 0.03 mmol) was added. The reaction mixture was evacuated under vacuum and backfilled with nitrogen. This process was repeated twice and then a balloon filled with hydrogen was placed on the flask and the reaction mixture was stirred for 30 minutes. The reaction mixture was filtered through a plug of celite and washed with methanol. The solvent was evaporated and the crude product was purified via silica gel column chromatography using 0 to 60% ethyl acetate in hexanes to obtain 2-(4-tert-butyl-2-methyl-phenyl)-5-isopentyloxy-1H-1,6-naphthyridin-4-one (315, 4 mg, 21%). ESI-MS m/z calc. 378.23, found 379.7 (M+1)⁺. ¹H NMR (400 MHz, DMSO-d₆) δ (ppm) 11.03 (d, J=6.0 Hz, 1H), 7.40 (d, J=7.8 Hz, 1H), 7.35-7.27 (m, 3H), 7.05 (s, 1H), 6.41 (dd, J=7.2, 1.3 Hz, 1H), 4.20 (t, J=6.7 Hz, 2H), 2.37 (s, 3H), 1.90 (dq, J=13.4, 6.7 Hz, 1H), 1.68 (q, J=6.8 Hz, 2H), 1.32 (s, 9H), 0.94 (d, J=6.6 Hz, 6H).

### Example 16

### 2-(4-tert-butyl-2-methyl-phenyl)-5-(2-methoxyethoxy)-1H-1,6-naphthyridin-4-one (316)

### Step 1: 2-(4-tert-butyl-2-methyl-phenyl)-5-(2-methoxyethoxy)-1H-1,6-naphthyridin-4-one (316)

2-(4-tert-butyl-2-methyl-phenyl)-5-(2-methoxyethoxy)-1H-1,6-naphthyridin-4-one was prepared using procedure analogous to that found in (316) Example 15 using 2-methoxyethanol instead. ESI-MS m/z calc. 366.19, found 367.93 (M+1)⁺.

### Example 17

### 3-chloro-2-[2-methyl-4-[1-(trifluoromethyl)cyclopropyl]phenyl]-1H-1,6-naphthyridin-4-one (317)

### Step 1: 3-chloro-2-[2-methyl-4-[1-(trifluoromethyl)cyclopropyl]phenyl]-1H-1,6-naphthyridin-4-one (317)

2-[2-methyl-4-[1-(trifluoromethyl)cyclopropyl]phenyl]-1H-1,6-naphthyridin-4-one (20 mg, 0.058 mmol) was suspended in DCM (1.5 mL) and NCS (10 mg, 0.075 mmol) was added. The reaction mixture was stirred for 3 weeks at RT. The solvent was evaporated and the residue was dissolved in DMF, filtered and purified by preparative reverse phase HPLC (C₁₈) using 1-99% ACN in water (supplemented with an HCl modifier) to obtain 3-chloro-2-[2-methyl-4-[1-(trifluoromethyl)cyclopropyl]phenyl]-1H-1,6-naphthyridin-4-one (317, 2.0 mg, 9%) ESI-MS m/z calc. 378.07, found 379.2 (M+1)⁺.

### Example 18

### 2-(4-tert-butyl-2,5-dimethyl-phenyl)-6-fluoro-5-hydroxy-1H-quinolin-4-one (318)

### Step 1: 2-(4-tert-butyl-2,5-dimethyl-phenyl)-6-fluoro-5-hydroxy-1H-quinolin-4-one (318)

To a solution of 2-(4-tert-butyl-2,5-dimethyl-phenyl)-6-fluoro-5-methoxy-1H-quinolin-4-one (25 mg, 0.07 mmol) in DCM (500 μL) cooled to −70° C. was added boron tribromide (350 μL of 1 M, 0.35 mmol) slowly under an atmosphere of nitrogen. The reaction mixture was stirred for 30 minutes at this temperature and quenched with methanol and stirred for 15 minutes at −70° C. The solvent was evaporated, and the crude material was taken up in methanol and purified via reverse phase column chromatography using 1 to 70% ACN in water (supplemented with an HCl modifier) to obtain 2-(4-tert-butyl-2,5-dimethyl-phenyl)-6-fluoro-5-hydroxy-1H-quinolin-4-one (318, 13 mg, 53%). ESI-MS m/z calc. 339.16, found 340.5 (M+1)⁺. ¹H NMR (400 MHz, DMSO-d₆) δ (ppm) 14.99 (d, J=0.9 Hz, 1H), 12.34 (s, 1H), 7.58 (dd, J=11.3, 9.1 Hz, 1H), 7.33 (s, 1H), 7.21 (s, 1H), 6.99 (dd, J=9.2, 3.6 Hz, 1H), 6.08 (d, J=1.5 Hz, 1H), 2.53 (s, 3H), 2.26 (s, 3H), 1.40 (s, 9H). ¹⁹F NMR (376 MHz, DMSO-d₆) δ (ppm) −145.95 (dd, J=11.2, 3.6 Hz).

### Example 19

### 6-fluoro-5-hydroxy-2-[2-methyl-4-(2,2,2-trifluoro-1,1-dimethyl-ethyl)phenyl]-1H-quinolin-4-one (319)

### Step 1: 6-fluoro-5-hydroxy-2-[2-methyl-4-(2,2,2-trifluoro-1,1-dimethyl-ethyl)phenyl]-1H-quinolin-4-one

6-fluoro-5-hydroxy-2-[2-methyl-4-(2,2,2-trifluoro-1,1-dimethyl-ethyl)phenyl]-1H-quinolin-4-one (319) was prepared from 6-fluoro-5-methoxy-2-[2-methyl-4-(2,2,2-trifluoro-1,1-dimethyl-ethyl)phenyl]-1H-quinolin-4-one using procedure analogous to that found in Example 18. ESI-MS m/z calc. 379.12, found 380.3 (M+1)⁺. ¹H NMR (400 MHz, DMSO-d₆) δ (ppm) 14.91 (s, 1H), 12.42 (s, 1H), 7.66-7.58 (m, 2H), 7.58-7.52 (m, 1H), 7.48 (d, J=8.1 Hz, 1H), 7.00 (dd, J=9.2, 3.6 Hz, 1H), 6.11 (d, J=1.5 Hz, 1H), 2.34 (s, 3H), 1.60 (s, 6H). ¹⁹F NMR (376 MHz, DMSO-d₆) δ (ppm) −74.65, −74.70, −145.79 (dd, J=11.2, 3.5 Hz).

### Example 20

### 2-(4-tert-butyl-2-methyl-phenyl)-7-fluoro-5-hydroxy-1H-quinolin-4-one (320)

### Step 1: 2-(4-tert-butyl-2-methyl-phenyl)-7-fluoro-5-hydroxy-1H-quinolin-4-one (320)

2-(4-tert-butyl-2-methyl-phenyl)-7-fluoro-5-hydroxy-1H-quinolin-4-one (320) was prepared from 2-(4-tert-butyl-2-methyl-phenyl)-7-fluoro-5-methoxy-1H-quinolin-4-one using procedure analogous to that found in Example 18. ESI-MS m/z calc. 325.15, found 326.66 (M+1)⁺. ¹H NMR (400 MHz, DMSO-d₆) δ (ppm) 15.12 (s, 1H), 12.31 (s, 1H), 7.44 (d, J=1.9 Hz, 1H), 7.43-7.34 (m, 2H), 6.70 (dd, J=10.5, 2.4 Hz, 1H), 6.48 (dd, J=11.1, 2.4 Hz, 1H), 6.10 (d, J=1.5 Hz, 1H), 2.31 (s, 3H), 1.32 (s, 9H).

### Example 21

### 2-(4-tert-butyl-2,5-dimethyl-phenyl)-7-fluoro-5-hydroxy-1H-quinolin-4-one (321)

### Step 1: 2-(4-tert-butyl-2,5-dimethyl-phenyl)-7-fluoro-5-hydroxy-1H-quinolin-4-one (321)

2-(4-tert-butyl-2,5-dimethyl-phenyl)-7-fluoro-5-hydroxy-1H-quinolin-4-one (321) was prepared from 2-(4-tert-butyl-2,5-dimethyl-phenyl)-7-fluoro-5-methoxy-1H-quinolin-4-one using procedure analogous to that found in Example 18. ESI-MS m/z calc. 339.16, found 340.5 (M+1)⁺. ¹H NMR (400 MHz, DMSO-d₆) δ (ppm) 15.13 (s, 1H), 12.29 (s, 1H), 7.33 (s, 1H), 7.20 (s, 1H), 6.71 (dd, J=10.4, 2.4 Hz, 1H), 6.48 (dd, J=11.1, 2.4 Hz, 1H), 6.10 (d, J=1.6 Hz, 1H), 2.53 (s, 3H), 2.26 (s, 3H), 1.40 (s, 9H).

### Example 22

### 7-fluoro-5-hydroxy-2-[2-methyl-4-(2,2,2-trifluoro-1,1-dimethyl-ethyl)phenyl]-1H-quinolin-4-one (322)

### Step 1: 7-fluoro-5-hydroxy-2-[2-methyl-4-(2,2,2-trifluoro-1,1-dimethyl-ethyl)phenyl]-1H-quinolin-4-one (322)

7-fluoro-5-hydroxy-2-[2-methyl-4-(2,2,2-trifluoro-1,1-dimethyl-ethyl)phenyl]-1H-quinolin-4-one (322) was prepared from 7-fluoro-5-methoxy-2-[2-methyl-4-(2,2,2-trifluoro-1,1-dimethyl-ethyl)phenyl]-1H-quinolin-4-one using procedure analogous to that found in Example 18. ESI-MS m/z calc. 379.12, found 380.3 (M+1)⁺. ¹H NMR (400 MHz, DMSO-d₆) δ (ppm) 15.07 (s, 1H), 12.37 (s, 1H), 7.60 (s, 1H), 7.59-7.52 (m, 1H), 7.48 (d, J=8.1 Hz, 1H), 6.71 (dd, J=10.5, 2.4 Hz, 1H), 6.49 (dd, J=11.1, 2.4 Hz, 1H), 6.13 (d, J=1.6 Hz, 1H), 2.34 (s, 3H), 1.60 (s, 6H).

### Example 23

### 2-(4-tert-butyl-2-methyl-phenyl)-6-fluoro-3-(methoxymethyl)-1H-quinolin-4-one (323)

### Step 1: ethyl 4-chloro-6-fluoro-1-oxido-quinolin-1-ium-3-carboxylate

To a flask charged with ethyl 4-chloro-6-fluoro-quinoline-3-carboxylate (2.5 g, 9.85 mmol) in DCM (40 mL), m-CPBA (3 g, 12.17 mmol) was added, and the reaction mixture was stirred for 2 hours at ambient temperature. The reaction was quenched with saturated aqueous sodium bicarbonate, filtered to remove insoluble ppt. The aqueous layer was extracted with DCM (3×), dried over magnesium sulfate, filtered and concentrated under reduced pressure to obtain ethyl 4-chloro-6-fluoro-1-oxido-quinolin-1-ium-3-carboxylate (2.6 g, 98%). ESI-MS m/z calc. 269.02, found 270.2 (M+1)⁺.

### Step 2: ethyl 2-bromo-4-chloro-6-fluoro-quinoline-3-carboxylate

To a flask charged with ethyl 4-chloro-6-fluoro-1-oxido-quinolin-1-ium-3-carboxylate (1.5 g, 5.56 mmol) in DCM (22 mL) was added phosphoryl tribromide (2.4 g, 8.37 mmol) and the reaction mixture was stirred at room temperature for 1 hour. The reaction mixture was poured over ice cold water and quenched with potassium carbonate. The aqueous layer was extracted with DCM (3×). The combined organic layer was dried over magnesium sulfate, filtered and concentrated. The crude material was purified via silica gel column chromatography using 0 to 10% ethyl acetate in hexanes to obtain ethyl 2-bromo-4-chloro-6-fluoro-quinoline-3-carboxylate (1.21 g, 65%) ESI-MS m/z calc. 330.94, found 334.1 (M+1)⁺. ¹H NMR (400 MHz, DMSO-d₆) δ (ppm) 8.22 (dd, J=9.2, 5.2 Hz, 1H), 8.01 (dd, J=9.3, 2.8 Hz, 1H), 7.99-7.91 (m, 1H), 4.50 (q, J=7.1 Hz, 2H), 1.47-1.27 (m, 3H).

### Step 3: ethyl 2-(4-tert-butyl-2-methyl-phenyl)-4-chloro-6-fluoro-quinoline-3-carboxylate

A microwave vial charged with ethyl 2-bromo-4-chloro-6-fluoro-quinoline-3-carboxylate (300 mg, 0.90 mmol), 2-(4-tert-butyl-2-methyl-phenyl)-4,4,5,5-tetramethyl-1,3,2-dioxaborolane (155 mg, 0.56 mmol), Pd(PPh₃)₄(16 mg, 0.014 mmol), Cs₂CO₃ (350 mg, 1.07 mmol), dioxane (6 mL) and water (600 μL) was degassed by bubbling nitrogen for 2-3 minutes. The vial was capped, and the reaction mixture was heated at 75° C. for 16 hours. The reaction mixture was filtered, and the solvent was evaporated. The crude material was purified via silica gel column chromatography using hexanes to obtain ethyl 2-(4-tert-butyl-2-methyl-phenyl)-4-chloro-6-fluoro-quinoline-3-carboxylate (325 mg, 90%) ESI-MS m/z calc. 399.14, found 400.56 (M+1)⁺. ¹H NMR (400 MHz, DMSO-d₆) δ (ppm) 8.21 (dd, J=9.2, 5.4 Hz, 1H), 8.04 (dd, J=9.5, 2.8 Hz, 1H), 7.96-7.87 (m, 1H), 7.36 (d, J=2.0 Hz, 1H), 7.29 (dd, J=8.0, 2.0 Hz, 1H), 7.14 (d, J=8.0 Hz, 1H), 4.08 (q, J=7.1 Hz, 2H), 2.13 (s, 3H), 1.32 (s, 9H), 0.80 (t, J=7.1 Hz, 3H).

### Step 4: [2-(4-tert-butyl-2-methyl-phenyl)-4-chloro-6-fluoro-3-quinolyl]methanol

To ethyl 2-(4-tert-butyl-2-methyl-phenyl)-4-chloro-6-fluoro-quinoline-3-carboxylate (100 mg, 0.25 mmol) in THF (2 mL) was added DIBAL (3 mL of 1 M, 3 mmol) at 0° C. The mixture was stirred at 0° C. for 10 min, and then at room temperature overnight. The reaction was worked up using Fieser work up-diluted with 2 mL ether and cooled to 0° C., added 0.04 mL water, followed by addition of 0.04 mL of 15% NaOH, 0.1 mL of water, stirred at room temperature for 15 minutes, added magnesium sulfate, stirred for 15 minutes, filtered and concentrated. Purified via silica gel column chromatography using 0 to 40% ethyl acetate in hexanes to obtain [2-(4-tert-butyl-2-methyl-phenyl)-4-chloro-6-fluoro-3-quinolyl]methanol (50 mg, 56%) ESI-MS m/z calc. 357.13, found 360.2 (M+3)⁺.

### Step 5: 2-(4-tert-butyl-2-methyl-phenyl)-4-chloro-6-fluoro-3-(methoxymethyl)quinoline

To a solution of [2-(4-tert-butyl-2-methyl-phenyl)-4-chloro-6-fluoro-3-quinolyl]methanol (50 mg, 0.14 mmol) in THF (1 mL) was added sodium hydride (12 mg of 60% w/w, 0.30 mmol) and the reaction mixture was stirred at room temperature for 1 hour. To it was added iodomethane (26 μL, 0.42 mmol) and the reaction mixture was stirred for 16 h at room temperature. The reaction mixture was quenched with water and the aqueous layer was extracted with DCM (3×). The combined organic layer was dried over magnesium sulfate, filtered and concentrated. Purification via silica gel column chromatography using 0 to 10% ethyl acetate in hexanes gave 2-(4-tert-butyl-2-methyl-phenyl)-4-chloro-6-fluoro-3-(methoxymethyl)quinoline (43 mg, 83%). ESI-MS m/z calc. 371.14, found 372.3 (M+1)⁺. ¹H NMR (400 MHz, DMSO-d₆) δ (ppm) 8.13 (dd, J=9.2, 5.4 Hz, 1H), 8.00 (dd, J=9.8, 2.8 Hz, 1H), 7.84 (td, J=8.8, 2.9 Hz, 1H), 7.37 (d, J=2.0 Hz, 1H), 7.32 (dd, J=8.0, 2.0 Hz, 1H), 7.21 (d, J=8.0 Hz, 1H), 4.42 (s, 2H), 3.13 (s, 3H), 2.04 (s, 3H), 1.35 (s, 9H).

### Step 6: 4-benzyloxy-2-(4-tert-butyl-2-methyl-phenyl)-6-fluoro-3-(methoxymethyl)quinoline

A round bottom flask equipped with a stir bar was charged with benzyl alcohol (17 μL, 0.16 mmol) and DMF (2 mL) followed by the addition of sodium hydride (8 mg, 0.33 mmol) and the reaction was stirred for 30 minutes at room temperature. 2-(4-tert-butyl-2-methyl-phenyl)-4-chloro-6-fluoro-3-(methoxymethyl)quinoline (40 mg, 0.11 mmol) was added as a solution in DMF (2 mL). The reaction was stirred for 3 h. and quenched with water and the aqueous layer was extracted with ethyl acetate (3×). The combined organic layer was washed with brine, dried over magnesium sulfate, filtered and concentrated. Purification via silica gel column chromatography using 0 to 15% ethyl acetate in hexanes gave 4-benzyloxy-2-(4-tert-butyl-2-methyl-phenyl)-6-fluoro-3-(methoxymethyl)quinoline (33 mg, 69%). ESI-MS m/z calc. 443.23, found 444.4 (M+1)⁺. ¹H NMR (400 MHz, DMSO-d₆) δ (ppm) 8.05 (dd, J=9.2, 5.3 Hz, 1H), 7.79-7.66 (m, 2H), 7.58-7.48 (m, 2H), 7.48-7.37 (m, 3H), 7.36-7.27 (m, 2H), 7.20 (d, J=8.0 Hz, 1H), 5.30 (s, 2H), 4.24 (s, 2H), 3.03 (s, 3H), 2.03 (s, 3H), 1.34 (s, 9H).

### Step 7: 2-(4-tert-butyl-2-methyl-phenyl)-6-fluoro-3-(methoxymethyl)-1H-quinolin-4-one (323) F

A vial charged with 4-benzyloxy-2-(4-tert-butyl-2-methyl-phenyl)-6-fluoro-3-(methoxymethyl)quinoline (30 mg, 0.07 mmol), Pd/C (10 mg, 0.09 mmol) and ethanol (1 mL) was degassed under vacuum and backfilled with nitrogen (twice). The reaction mixture was again degassed under vacuum and a balloon filled with hydrogen was placed on the reaction mixture and was stirred for 30 minutes. The reaction mixture was filtered through a plug of celite and washed with methanol. The solvent was evaporated and the crude material was purified via reverse phase column chromatography (C₁₈) using 1 to 99% ACN in water (HCl modifier) to obtain 2-(4-tert-butyl-2-methyl-phenyl)-6-fluoro-3-(methoxymethyl)-1H-quinolin-4-one (323, 9 mg, 37%). ESI-MS m/z calc. 353.18, found 355.2 (M+2)⁺. H NMR (400 MHz, DMSO-d₆) δ (ppm) 11.87 (s, 1H), 7.78 (dd, J=9.4, 3.0 Hz, 1H), 7.64 (dd, J=9.1, 4.8 Hz, 1H), 7.57 (td, J=8.6, 2.9 Hz, 1H), 7.43 (d, J=2.0 Hz, 1H), 7.37 (dd, J=8.1, 2.0 Hz, 1H), 7.29 (d, J=8.0 Hz, 1H), 4.14 (d, J=10.1 Hz, 1H), 3.89 (d, J=10.1 Hz, 1H), 3.06 (s, 3H), 2.17 (s, 3H), 1.34 (s, 9H).

### Example 24

### 2-(4-tert-butyl-2-methyl-phenyl)-5-(methylamino)-1,6-naphthyridin-4-ol (Hydrochloride salt) (324)

### Step 1: 2-(4-tert-butyl-2-methyl-phenyl)-5-chloro-1,6-naphthyridin-4-ol

In a 4-mL vial, 2-(4-tert-butyl-2-methyl-phenyl)-4-[(4-methoxyphenyl)methoxy]-6-oxido-1,6-naphthyridin-6-ium (104 mg, 0.19 mmol) was dissolved in POCl₃ (200 μL, 2.15 mmol) and stirred for 5 min. It was then cooled to 0° C. and quenched with methanol (500 μL). This mixture was filtered and purified by reverse-phase preparative chromatography (C₁₈) using a gradient eluent of 1 to 99% acetonitrile in water (HCl modifier) to give 2-(4-tert-butyl-2-methyl-phenyl)-5-chloro-1,6-naphthyridin-4-ol (14.5 mg, 23%). ESI-MS m/z calc. 326.12, found 327.3 (M+1)⁺. ¹H NMR (400 MHz, CDCl₃) δ (ppm) 10.47-10.17 (br s, 1H), 7.54 (s, 1H), 7.47 (d, J=8.0 Hz, 1H), 7.44-7.38 (m, 1H), 7.38-7.32 (m, 2H), 7.21-7.11 (m, 1H), 2.47 (s, 3H), 1.35 (s, 9H)

### Step 2: 2-(4-tert-butyl-2-methyl-phenyl)-5-(methylamino)-1,6-naphthyridin-4-ol (324)

In a 4-mL vial, 2-(4-tert-butyl-2-methyl-phenyl)-5-chloro-1,6-naphthyridin-4-ol (12 mg, 0.035 mmol) was dissolved in NMP (300 μL), to which a THF solution of methylamine (100 μL of 2.0 M, 0.20 mmol) was added. The resulting mixture was stirred at 100° C. for 4.5 h, after which it was cooled to room temperature, diluted with methanol (500 μL), filtered, and purified by reverse-phase preparative chromatography (C₁₈) using a gradient eluent of 1 to 70% acetonitrile in water containing 5 mM HCl solution to give 2-(4-tert-butyl-2-methyl-phenyl)-5-(methylamino)-1,6-naphthyridin-4-ol (Hydrochloride salt) (324, 9.0 mg, 71%). ESI-MS m/z calc. 321.18, found 322.4 (M+1)⁺. ¹H NMR (400 MHz, DMSO-d₆) δ (ppm) 13.54-13.28 (m, 1H), 12.39 (s, 1H), 10.67 (q, J=5.2 Hz, 1H), 7.76 (t, J=6.7 Hz, 1H), 7.48 (s, 1H), 7.45 (d, J=1.2 Hz, 2H), 6.84 (s, 1H), 6.72-6.59 (m, 1H), 3.12 (d, J=5.2 Hz, 3H), 2.35 (s, 3H), 1.33 (s, 9H).

### Example 25

### 2-[2,5-dimethyl-4-(2,2,2-trifluoro-1,1-dimethyl-ethyl)phenyl]-N-methyl-4-oxo-1H-1,6-naphthyridine-5-carboxamide (325)

### Step 1: 2-[2,5-dimethyl-4-(2,2,2-trifluoro-1,1-dimethyl-ethyl)phenyl]-4-oxo-1H-1,6-naphthyridine-5-carboxylic acid

To a suspension of 2-[2,5-dimethyl-4-(2,2,2-trifluoro-1,1-dimethyl-ethyl)phenyl]-4-oxo-1H-1,6-naphthyridine-5-carbonitrile (330 mg, 0.85 mmol) in ethanol (7 mL) was added KOH (670 μL of 10% w/v, 1.19 mmol) and water (480 μL of 30% w/v, 4.23 mmol). The reaction mixture was stirred at room temperature for 16 h. The reaction mixture was cooled to room temperature concentrated. The crude material was taken up in DCM washed with water, dried over magnesium sulfate, filtered and concentrated. The solvent was evaporated and the crude material was dissolved in DMSO/methanol and purified via reverse phase mass directed column chromatography using 1 to 70% ACN in water (HCl modifier) to obtain 2-[2,5-dimethyl-4-(2,2,2-trifluoro-1,1-dimethyl-ethyl)phenyl]-4-oxo-1H-1,6-naphthyridine-5-carboxylic acid (50 mg, 15%). ESI-MS m/z calc. 404.13 found 405.2 (M+1)⁺.

### Step 2: 2-[2,5-dimethyl-4-(2,2,2-trifluoro-1,1-dimethyl-ethyl)phenyl]-N-methyl-4-oxo-1H-1,6-naphthyridine-5-carboxamide (325)

To a solution of 2-[2,5-dimethyl-4-(2,2,2-trifluoro-1,1-dimethyl-ethyl)phenyl]-4-oxo-1H-1,6-naphthyridine-5-carboxylic acid (10 mg, 0.025 mmol) in DCM (200 μL) was added methanamine (Hydrochloride salt) (4 mg, 0.06 mmol), HATU (12 mg, 0.03 mmol) followed by the addition of DIEA (12 μL, 0.07 mmol). The reaction mixture was stirred at room temperature for 14 hours and the solvent was evaporated. The crude product was purified via silica gel column chromatography using 0 to 100% ethyl acetate in hexanes to obtain 2-[2,5-dimethyl-4-(2,2,2-trifluoro-1,1-dimethyl-ethyl)phenyl]-N-methyl-4-oxo-1H-1,6-naphthyridine-5-carboxamide (325) (6 mg, 55%). ESI-MS m/z calc. 417.16, found 418.6 (M+1)⁺. ¹H NMR (400 MHz, DMSO-d₆) δ (ppm) 12.01 (s, 1H), 8.50 (d, J=5.7 Hz, 1H), 7.92 (d, J=4.9 Hz, 1H), 7.54 (s, 1H), 7.46 (d, J=5.8 Hz, 1H), 7.30 (s, 1H), 6.07 (s, 1H), 2.76 (d, J=4.7 Hz, 3H), 2.55 (s, 3H), 2.30 (s, 3H), 1.72 (s, 6H). ¹⁹F NMR (376 MHz, DMSO-d₆) δ (ppm) −73.86.

### Example 26

### 2-[2,5-dimethyl-4-(2,2,2-trifluoro-1,1-dimethyl-ethyl)phenyl]-N,N-dimethyl-4-oxo-1H-1,6-naphthyridine-5-carboxamide (326)

### Step 1: 2-[2,5-dimethyl-4-(2,2,2-trifluoro-1,1-dimethyl-ethyl)phenyl]-N,N-dimethyl-4-oxo-1H-1,6-naphthyridine-5-carboxamide (326)

To a solution of 2-[2,5-dimethyl-4-(2,2,2-trifluoro-1,1-dimethyl-ethyl)phenyl]-4-oxo-1H-1,6-naphthyridine-5-carboxylic acid (10 mg, 0.025 mmol) in DCM (300 μL) was added N-methylmethanamine (Hydrochloride salt) (4 mg, 0.05 mmol), followed by the addition of HATU (11 mg, 0.03 mmol) and DIEA (15 μL, 0.09 mmol). The reaction mixture was stirred at room temperature for 10 minutes and the solvent was evaporated. The crude product was purified via silica gel column chromatography using 0 to 100% ethyl acetate in hexanes to obtain 2-[2,5-dimethyl-4-(2,2,2-trifluoro-1,1-dimethyl-ethyl)phenyl]-N,N-dimethyl-4-oxo-1H-1,6-naphthyridine-5-carboxamide (326, 8 mg, 75%). ESI-MS m/z calc. 431.18, found 432.5 (M+1)⁺. ¹H NMR (400 MHz, DMSO-d₆) δ (ppm) 12.19 (s, 1H), 8.56 (d, J=5.9 Hz, 1H), 7.54 (s, 1H), 7.51 (d, J=5.9 Hz, 1H), 7.32 (s, 1H), 6.12 (s, 1H), 3.00 (s, 3H), 2.64 (s, 3H), 2.54 (s, 3H), 2.31 (s, 3H), 1.72 (s, 6H).

### Example 27

### 2-(4-tert-butyl-2-methyl-phenyl)-4-oxo-1H-1,6-naphthyridine-5-carbothioamide (327)

### Step 1: 2-(4-tert-butyl-2-methyl-phenyl)-4-oxo-1H-1,6-naphthyridine-5-carbothioamide (327)

To a solution of 2-(4-tert-butyl-2-methyl-phenyl)-4-[(4-methoxyphenyl)methoxy]-1,6-naphthyridine-5-carbonitrile (16 mg, 0.036 mmol) in methanol (500 μL) was added ammonium sulfide solution in water (6 μL of 45% w/v, 0.04 mmol). The reaction mixture was subjected to microwave irradiation at 120° C. for 30 minutes. The reaction mixture was concentrated and purified via revere phase column chromatography using 1 to 60% ACN in water (supplemented with an HCl modifier) to obtain 2-(4-tert-butyl-2-methyl-phenyl)-4-oxo-1H-1,6-naphthyridine-5-carbothioamide (327, 5 mg, 37%). ESI-MS m/z calc. 351.14, found 352.59 (M+1)⁺. ¹H NMR (400 MHz, DMSO-d₆) δ (ppm) 12.29 (s, 1H), 10.21 (s, 1H), 9.76 (s, 1H), 8.49 (dd, J=6.1, 1.5 Hz, 1H), 7.54 (s, 1H), 7.45 (d, J=1.9 Hz, 1H), 7.43-7.36 (m, 2H), 6.16 (d, J=2.2 Hz, 1H), 2.34 (s, 3H), 1.33 (s, 9H).

### Example 28

### 2-(4-tert-butyl-2-methyl-phenyl)-6-methyl-1H-1,6-naphthyridin-6-ium-4-one (328)

### Step 1: 2-(4-tert-butyl-2-methyl-phenyl)-6-methyl-1H-1,6-naphthyridin-6-ium-4-one (328)

Sodium hydride (17 mg of 60% w/w, 0.42 mmol) was added to 2-(4-tert-butyl-2-methyl-phenyl)-1H-1,6-naphthyridin-4-one (46.0 mg) (92) in THF (2 mL) at 0° C. The reaction mixture was stirred at this temperature for 1 h. Iodomethane (50 μL, 0.80 mmol) was added, and the reaction mixture was stirred at room temperature for 1 h. The crude material was filtered and purified by reverse phase preparative column chromatography (C₁₈) using 10 to 60% acetonitrile in water containing 5 mM hydrochloric acid to give 2-(4-tert-butyl-2-methyl-phenyl)-6-methyl-1H-1,6-naphthyridin-6-ium-4-one (328, 15.3 mg, 18%). ESI-MS m/z calc. 307.18, found 307.3 (M+0)⁺. ¹H NMR (400 MHz, DMSO-d₆) δ (ppm) 13.29 (s, 1H), 8.72 (dd, J=7.0, 1.5 Hz, 1H), 8.36 (d, J=1.5 Hz, 1H), 7.86 (d, J=7.0 Hz, 1H), 7.45 (d, J=2.0 Hz, 1H), 7.40 (dd, J=7.9, 2.0 Hz, 1H), 7.23 (d, J=8.0 Hz, 1H), 6.66 (s, 1H), 4.21 (s, 3H), 2.18 (s, 3H), 1.37 (s, 9H).

### Example 29

### 2-(4-tert-butyl-5-chloro-2-methyl-phenyl)-5-hydroxy-1H-1,7-naphthyridin-4-one (329)

### Step 1: 2-(4-tert-butyl-5-chloro-2-methyl-phenyl)-5-hydroxy-1H-1,7-naphthyridin-4-one (329)

A solution of 2-(4-tert-butyl-5-chloro-2-methyl-phenyl)-5-methoxy-1H-1,7-naphthyridin-4-one (17 mg, 0.05 mmol) in DCM (2 mL) was treated with boron tribromide (500 μL of 0.1 M, 0.05 mmol) at −20° C. The reaction mixture was gradually warmed to room temperature and stirred for 1 h and quenched with DCM. The solvent was evaporated in vacuo, dissolved in methanol and purified by preparative reverse phase HPLC (C₁₈) using 1-99% ACN in water (supplemented with an HCl modifier) to give 2-(4-tert-butyl-5-chloro-2-methyl-phenyl)-5-hydroxy-1H-1,7-naphthyridin-4-one (329, 5.0 mg, 31%) ESI-MS m/z calc. 342.11, found 343.3 (M+1)⁺. ¹H NMR (400 MHz, CD₃OD) δ (ppm) 8.41 (s, 1H), 7.98 (s, 1H), 7.51 (s, 1H), 7.49 (s, 1H), 6.29 (s, 1H), 2.35 (s, 3H), 1.53 (s, 9H).

### Example 30

### 2-(4-tert-butyl-2-methyl-phenyl)-4-oxo-1H-quinoline-6-carbonitrile (330)

### Step 1: 2-(4-tert-butyl-2-methyl-phenyl)-4-oxo-1H-quinoline-6-carbonitrile (330)

To a solution of 4-tert-butyl-2-methyl-benzoic acid (110 mg, 0.57 mmol) in dry DCM (7 mL) and DMF (0.1 mL) was added oxalyl dichloride (110 mg, 0.87 mmol) at 0° C. The reaction mixture was stirred at room temperature for 1 h and refluxed for 5 min. The solvent was evaporated and the obtained acyl chloride was added to a solution of 1-(2-amino-5-bromo-phenyl)ethanone (101 mg, 0.47 mmol) and Et₃N (290 mg, 2.87 mmol) in DMF (3 mL). The reaction mixture was stirred at room temperature overnight, filtered and purified by preparative reverse phase HPLC (C₁₈) using 1 to 99% ACN in water (supplemented with an HCl modifier) to give N-(2-acetyl-4-bromo-phenyl)-4-tert-butyl-2-methyl-benzamide (22.8 mg, 12%). ESI-MS m/z calc. 387.08, found 390.2 (M+3)⁺. It was dissolved in dioxane (1.5 mL) and treated with NaOH (10 mg, 0.25 mmol) powder. The reaction mixture was sparged with nitrogen, sealed and stirred at 120° C. for 2 h. The reaction mixture was filtered and purified by preparative reverse phase HPLC (C₁₈) using 1 to 99% ACN in water (supplemented with an HCl modifier) to give 6-bromo-2-(4-tert-butyl-2-methyl-phenyl)-1H-quinolin-4-one (6.9 mg, 4%). ESI-MS m/z calc. 369.07, found 372.1 (M+3)⁺. It was dissolved in DMF (1 mL) and treated with dicyanozinc (8 mg, 0.07 mmol) and Pd(dppf) (5 mg, 0.006 mmol). The reaction mixture was sparged with nitrogen, sealed and stirred at 140° C. overnight. The mixture was filtered and purified by preparative reverse phase HPLC (C₁₈) using 1 to 99% ACN in water (supplemented with an HCl modifier) to give 2-(4-tert-butyl-2-methyl-phenyl)-4-oxo-1H-quinoline-6-carbonitrile (330, 5.2 mg, 3%). ESI-MS m/z calc. 316.16, found 317.3 (M+1)⁺. ¹H NMR (400 MHz, DMSO-d₆) δ (ppm) 12.12 (s, 1H), 8.46 (d, J=2.0 Hz, 1H), 8.01 (dd, J=8.6, 2.0 Hz, 1H), 7.71 (d, J=8.7 Hz, 1H), 7.44 (s, 1H), 7.39 (s, 2H), 6.11 (s, 1H), 2.32 (s, 3H), 1.33 (s, 9H).

### Example 31

### 2-(4-tert-butyl-5-chloro-2-methyl-phenyl)-6-oxido-4-oxo-1H-1,6-naphthyridin-6-ium-5-carboxamide (331)

### Step 1: 4-benzyloxy-2-chloro-6-oxido-1,6-naphthyridin-6-ium-5-carbonitrile

To a flask charged with 4-benzyloxy-2-chloro-1,6-naphthyridine-5-carbonitrile (100 mg, 0.34 mmol) in DCM (2 mL), 3-chlorobenzenecarboperoxoic acid (170 mg, 0.69 mmol) was added and stirred for 3 days at ambient temperature. The reaction mixture was quenched with saturated sodium bicarbonate solution and filtered. The aqueous layer was extracted with DCM (3×), dried over magnesium sulfate, filtered and concentrated under reduced pressure. Purification via silica gel column chromatography using 0 to 100% ethyl acetate in hexanes gave 4-benzyloxy-2-chloro-6-oxido-1,6-naphthyridin-6-ium-5-carbonitrile (30 mg, 28%) ESI-MS m/z calc. 311.05, found 312.1 (M+1)⁺. ¹H NMR (400 MHz, DMSO-d₆) δ (ppm) 8.61 (d, J=7.4 Hz, 1H), 8.09 (d, J=7.4 Hz, 1H), 7.61-7.57 (m, 3H), 7.48-7.34 (m, 3H), 5.57 (s, 2H).

### Step 2: 2-(4-tert-butyl-5-chloro-2-methyl-phenyl)-6-oxido-4-oxo-1H-1,6-naphthyridin-6-ium-5-carboxamide (331)

4-benzyloxy-2-chloro-6-oxido-1,6-naphthyridin-6-ium-5-carbonitrile (30 mg, 0.10 mmol), 2-(4-tert-butyl-5-chloro-2-methyl-phenyl)-4,4,5,5-tetramethyl-1,3,2-dioxaborolane (30 mg, 0.10 mmol), and aqueous potassium phosphate (250 μL of 1 M, 0.25 mmol) were combined in dioxane (500 μL) and purged with nitrogen for 10 minutes. PdCl₂(dtbpf) (10 mg, 0.01 mmol) was added, and the reaction was purged with nitrogen for an additional 5 minutes and stirred at room temperature for 30 minutes. The reaction was partitioned between ethyl acetate and water. The organics were separated, washed with brine, dried over magnesium sulfate and evaporated. The crude material was purified via silica gel column chromatography using 0 to 100% ethyl acetate in hexanes to give 4-benzyloxy-2-(4-tert-butyl-5-chloro-2-methyl-phenyl)-6-oxido-1,6-naphthyridin-6-ium-5-carbonitrile (15 mg, 34%) as a white solid. ESI-MS m/z calc. 457.16, found 458.5 (M+1)⁺. It was taken up in toluene (500 μL) and TFA (360 μL, 4.67 mmol). The resulting reaction mixture was heated at 60° C. for 15 h. The solvent was evaporated, and the crude material was purified via reverse phase column chromatography using 1 to 50% ACN in water (supplemented with an HCl modifier) to obtain 2-(4-tert-butyl-5-chloro-2-methyl-phenyl)-6-oxido-4-oxo-1H-1,6-naphthyridin-6-ium-5-carboxamide (331, 8 mg, 22%). ESI-MS m/z calc. 385.12, found 386.4 (M+1). ¹H NMR (400 MHz, DMSO-d₆) δ (ppm) 12.24 (s, 1H), 8.35 (d, J=7.3 Hz, 1H), 7.77 (s, 1H), 7.61 (s, 1H), 7.56 (d, J=7.3 Hz, 1H), 7.50 (d, J=6.6 Hz, 2H), 6.13 (s, 1H), 2.31 (s, 3H), 1.48 (s, 9H).

### Example 32

### rac-2-[2,5-dimethyl-4-(2,2,2-trifluoro-1-methyl-ethyl)phenyl]-6-fluoro-1H-quinolin-4-one (332)

### Step 1: 2-[2,5-dimethyl-4-(2,2,2-trifluoro-1-methyl-ethyl)phenyl]-6-fluoro-1H-quinolin-4-one (332)

A flask charged with 2-[2,5-dimethyl-4-[1-(trifluoromethyl)vinyl]phenyl]-6-fluoro-1H-quinolin-4-one (15 mg, 0.04 mmol), Pd on carbon (5 mg, 0.05 mmol) and ethanol (1 mL) was degassed under vacuum and backfilled with nitrogen. This process was repeated twice, and a balloon filled with hydrogen was placed on the reaction mixture and stirred for 16 h. The reaction mixture was filtered and washed with methanol. The solvent was evaporated, and the crude material was taken up in DMSO and purified via reverse phase column chromatography (C₁₈) using 1 to 99% ACN in water (HCl modifier) to obtain rac-2-[2,5-dimethyl-4-(2,2,2-trifluoro-1-methyl-ethyl)phenyl]-6-fluoro-1H-quinolin-4-one (332, 5 mg, 32%). ESI-MS m/z calc. 363.12463, found 364.5 (M+1). ¹H NMR (400 MHz, DMSO-d₆) δ (ppm) 12.28 (s, 1H), 7.80 (dd, J=9.3, 3.0 Hz, 1H), 7.73 (dd, J=9.1, 4.7 Hz, 1H), 7.64 (td, J=8.6, 3.0 Hz, 1H), 7.43 (s, 1H), 7.35 (s, 1H), 6.15 (s, 1H), 2.38 (s, 3H), 2.29 (s, 3H), 1.49 (d, J=7.1 Hz, 3H), (H buried under broad water peak). ¹⁹F NMR (376 MHz, DMSO-d₆) δ (ppm) −69.45 (d, J=9.5 Hz), −117.00.

### Example 33

### (R)-2-[2,5-dimethyl-4-(2,2,2-trifluoro-1-methyl-ethyl)phenyl]-6-fluoro-1H-quinolin-4-one and (S)-2-[2,5-dimethyl-4-(2,2,2-trifluoro-1-methyl-ethyl)phenyl]-6-fluoro-1H-quinolin-4-one (333 & 334)

### Step 1: (R)-2-[2,5-dimethyl-4-(2,2,2-trifluoro-1-methyl-ethyl)phenyl]-6-fluoro-1H-quinolin-4-one (333) and (S)-2-[2,5-dimethyl-4-(2,2,2-trifluoro-1-methyl-ethyl)phenyl]-6-fluoro-1H-quinolin-4-one (334)

rac-2-[2,5-dimethyl-4-(2,2,2-trifluoro-1-methyl-ethyl)phenyl]-6-fluoro-1H-quinolin-4-one (15 mg, 0.04 mmol) was separated using chiral SFC using a ChiralPak AS (10×250 mm, 5 um) column at 50° C. with a mobile phase of 12% methanol (20 mM NH₃), 88% CO₂ at 70 mL/min flow. Concentration of the sample was 15.5 mg/mL (in 91% methanol+9% DMSO), injection volume was 100 μL, outlet pressure at 170 bar, and detection wavelength 210 nm. Minute run) to give: Enantiomer 1 (Peak 1 SFC) 1: 2-[2,5-dimethyl-4-(2,2,2-trifluoro-1-methyl-ethyl)phenyl]-6-fluoro-1H-quinolin-4-one (333, 5 mg, 33%). ESI-MS m/z calc. 363.12, found 364.1 (M+1)⁺. 2-[2,5-dimethyl-4-(2,2,2-trifluoro-1-methyl-ethyl)phenyl]-6-fluoro-1H-quinolin-4-one (5 mg, 330%) ¹H NMR (400 MHz, DMSO-d₆) δ (ppm) 11.94 (s, 1H), 7.75 (dd, J=9.4, 3.0 Hz, 1H), 7.66 (dd, J=9.1, 4.7 Hz, 1H), 7.56 (td, J=8.5, 2.8 Hz, 1H), 7.40 (s, 1H), 7.32 (s, 1H), 6.00 (s, 1H), 4.03 (p, J=8.1 Hz, 1H), 2.37 (s, 3H), 2.29 (s, 3H), 1.49 (d, J=7.1 Hz, 3H). ¹⁹F NMR (376 MHz, DMSO-d₆) δ (ppm) −69.47 (d, J=9.6 Hz), −118.00; and Enantiomer 2 (Peak 2 SFC): 2-[2,5-dimethyl-4-(2,2,2-trifluoro-1-methyl-ethyl)phenyl]-6-fluoro-1H-quinolin-4-one (334, 5.3 mg, 35%) ESI-MS m/z calc. 363.12, found 364.1 (M+1)⁺. ¹H NMR (400 MHz, DMSO-d₆) δ (ppm) 11.98 (s, 1H), 7.72 (dd, J=9.6, 3.0 Hz, 1H), 7.61 (dd, J=9.0, 4.8 Hz, 1H), 7.46 (s, 1H), 7.35 (s, 1H), 7.28 (s, 1H), 5.97 (s, 1H), 4.00 (t, J=8.4 Hz, 1H), 2.36 (s, 3H), 2.28 (s, 3H), 1.48 (d, J=7.1 Hz, 3H).

### Example 34

### 2-[4-(1-hydroxy-1-methyl-ethyl)-2-methyl-phenyl]-1H-quinolin-4-one (335)

### Step 1: 4-benzyloxy-2-(4-isopropenyl-2-methyl-phenyl)quinoline and 2-[4-(4-benzyloxy-2-quinolyl)-3-methyl-phenyl]propan-2-ol

In a 4-mL vial, ethyl 4-(4-benzyloxy-2-quinolyl)-3-methyl-benzoate (22.2 mg, 0.06 mmol) was mixed with THF (500 μL), to which an ether solution of methyl magnesium bromide (80 μL of 3.0 M, 0.24 mmol) was added dropwise. The resulting mixture was stirred at room temperature for 2.5 h, after which it was quenched with ethanol (500 μL), filtered, and purified by reverse-phase preparative chromatography (C₁₈) using 1 to 70% acetonitrile in water containing 5 mM HCl solution to give a white solid that was a ˜1:1 mixture of 4-benzyloxy-2-(4-isopropenyl-2-methyl-phenyl)quinoline ESI-MS m/z calc. 383.19, found 384.5 (M+1)⁺ 2-[4-(4-benzyloxy-2-quinolyl)-3-methyl-phenyl]propan-2-ol (16.3 mg, 78%) ESI-MS m/z calc. 365.47, found 366.5 (M+1)⁺.

### Step 2: 2-[4-(1-hydroxy-1-methyl-ethyl)-2-methyl-phenyl]-1H-quinolin-4-one (335)

In a 4-mL vial, a mixture of 4-benzyloxy-2-(4-isopropenyl-2-methyl-phenyl)quinoline and 2-[4-(4-benzyloxy-2-quinolyl)-3-methyl-phenyl]propan-2-ol (16.3 mg, 0.022 mmol) was suspended in ethanol (500 μL) and THF (500 μL), and this mixture was sparged with nitrogen gas for 5 min. Pd/C (2.9 mg of 10% w/w, 0.003 mmol) was added, and the mixture was stirred at room temperature under a balloon of hydrogen for 14 h. It was then diluted with methanol (500 μL), filtered, and purified by reverse-phase preparative chromatography (C₁₈) column using 1 to 70% acetonitrile in water containing 5 mM HCl solution to give 2-[4-(1-hydroxy-1-methyl-ethyl)-2-methyl-phenyl]-1H-quinolin-4-one (335, 4.6 mg, 72%); ESI-MS m/z calc. 293.14157, found 294.4 (M+1)⁺. ¹H NMR (400 MHz, DMSO-d₆) δ (ppm) 11.75 (s, 1H), 8.11 (dd, J=8.1, 1.5 Hz, 1H), 7.65 (ddd, J=8.4, 6.8, 1.5 Hz, 1H), 7.59 (d, J=8.2 Hz, 1H), 7.49 (d, J=1.8 Hz, 1H), 7.44 (dd, J=8.0, 1.8 Hz, 1H), 7.35 (d, J=8.0 Hz, 1H), 7.33 (ddd, J=8.1, 6.8, 1.2 Hz, 1H), 5.97 (d, J=1.7 Hz, 1H), 5.11 (s, 1H), 2.31 (s, 3H), 1.46 (s, 6H); and 2-(4-isopropyl-2-methylphenyl)quinolin-4(1H)-one.

### Example 35

### 2-(5-bromo-6-tert-butyl-2-chloro-3-pyridyl)-1H-quinolin-4-one (336)

### Step 1: N-(2-acetylphenyl)-5-bromo-6-tert-butyl-2-chloro-pyridine-3-carboxamide

A solution of 5-bromo-6-tert-butyl-2-chloro-pyridine-3-carboxylic acid (107 mg, 0.37 mmol) and CDI (75 mg, 0.46 mmol) in DMF (5 mL) was stirred at 45° C. for 1 h. The reaction mixture was cooled to room temperature and added to a solution of 1-(2-aminophenyl)ethanone (62 mg, 0.46 mmol) and sodium hydride (20 mg of 60% w/w, 0.5 mmol) in DMF (5 mL) at 0° C. The reaction mixture was stirred overnight at room temperature and quenched with water. The reaction mixture was extracted with ethyl acetate, dried and purified by preparative reverse phase HPLC (C₁₈) using 30 to 99% ACN in water (supplemented with an HCl modifier) to give N-(2-acetylphenyl)-5-bromo-6-tert-butyl-2-chloro-pyridine-3-carboxamide (14.4 mg, 10%). ESI-MS m/z calc. 408.02, found 411.1 (M+1)⁺. ¹H NMR (400 MHz, DMSO-d₆) δ (ppm) 11.61 (s, 1H), 8.35 (s, 1H), 8.24 (d, J=8.2 Hz, 1H), 8.01 (d, J=9.4 Hz, 1H), 7.67 (td, J=8.4, 7.9, 1.6 Hz, 1H), 7.39-7.28 (m, 1H), 2.63 (s, 3H), 1.51 (s, 9H).

### Step 2: 2-(5-bromo-6-tert-butyl-2-chloro-3-pyridyl)-1H-quinolin-4-one (336)

In a 2 mL microwave vial a solution of N-(2-acetylphenyl)-5-bromo-6-tert-butyl-2-chloro-pyridine-3-carboxamide (14 mg, 0.03 mmol) and NaOH (5 mg, 0.12 mmol) in dioxane (1 mL) was sparged with nitrogen, sealed and heated at 120° C. for 1 h. The reaction mixture was filtered and purified by preparative reverse phase HPLC (C₁₈) using 1-99% ACN in water (supplemented with an HCl modifier) to give 2-(5-bromo-6-tert-butyl-2-chloro-3-pyridyl)-1H-quinolin-4-one (336, 5.2 mg, 37%). ESI-MS m/z calc. 390.01, found 393.1 (M+3)⁺.

### Example 36

### 2-(4-isopropyl-2-methoxy-6-methyl-phenyl)-1H-quinolin-4-one (337)

### Step 1: 2-(4-isopropyl-2-methoxy-6-methyl-phenyl)-1H-quinolin-4-one (337)

In a 1 dram vial a solution of 4-benzyloxy-2-(4-chloro-2-methoxy-6-methyl-phenyl)quinoline (95 mg, 0.24 mmol), Pd(OAc)₂ (2.2 mg, 0.01 mmol) and CPhos (8.6 mg, 0.02 mmol) in THF (2 mL) was sparged with nitrogen and sealed. THF solution of bromo(isopropyl)zinc (1000 μL of 0.5 M, 0.5 mmol) was added and the reaction mixture was stirred at room temperature for 48 h. The reaction mixture was quenched with a solution of ammonium chloride and extracted with ethyl acetate. The organic layer was dried, evaporated and purified by preparative reverse phase HPLC (C₁₈) using 1 to 99% ACN in water (supplemented with an HCl modifier) to give 4-benzyloxy-2-(4-isopropyl-2-methoxy-6-methyl-phenyl)quinoline (25 mg, 26%) ESI-MS m/z calc. 397.20, found 398.6 (M+1)⁺. It was taken up in ethanol (3 mL) was treated with Pd/C (7 mg of 10% w/w, 0.006 mmol) and sparged with hydrogen for 1 h at room temperature. The reaction mixture was filtered and purified by preparative reverse phase HPLC (C₁₈) using 1-99% ACN in water (supplemented with an HCl modifier) to give 2-(4-isopropyl-2-methoxy-6-methyl-phenyl)-1H-quinolin-4-one (337, 18 mg, 24%). ESI-MS m/z calc. 307.16, found 308.5 (M+1)⁺. ¹H NMR (400 MHz, DMSO-d₆) δ (ppm) 12.61 (s, 1H), 8.18 (d, J=8.2 Hz, 1H), 7.80-7.72 (m, 1H), 7.68 (d, J=8.3 Hz, 1H), 7.47 (t, J=7.5 Hz, 1H), 6.91 (d, J=9.9 Hz, 2H), 6.23 (s, 1H), 3.74 (s, 3H), 2.94 (p, J=6.9 Hz, 1H), 2.14 (s, 3H), 1.26 (d, J=6.9 Hz, 6H).

### Example 37

### 2-[4-(2,2-dimethylpropyl)-2-methyl-phenyl]-1H-quinolin-4-one (338)

### Step 1: 2-[4-(2,2-dimethylpropyl)-2-methyl-phenyl]-1H-quinolin-4-one (338)

In a 1 dram vial 4-benzyloxy-2-(4-chloro-2-methyl-phenyl)quinoline (47 mg, 0.13 mmol), CPhos (5 mg, 0.011 mmol), Pd(OAc)₂ (1.2 mg, 0.005 mmol) in THF (1.5 mL) was treated with bromo 2,2-dimethylpropylzinc (520 μL of 0.5 M, 0.26 mmol) at room temperature and was stirred at overnight. The reaction was quenched with a solution of ammonium chloride and extracted with ethyl acetate. The organic phase was dried and evaporated to obtain 4-benzyloxy-2-[4-(2,2-dimethylpropyl)-2-methyl-phenyl]quinoline (27.8 mg, 54%). ESI-MS m/z calc. 395.22, found 396.7 (M+1)⁺. The obtained intermediate was dissolved in methanol (3 mL) and Pd/C (14 mg of 10% w/w, 0.01316 mmol) was added. The mixture was sparged with hydrogen from a balloon and stirred at room temperature for 1 h. The mixture was filtered and purified by preparative reverse phase HPLC (C₁₈) using 1-99% ACN in water (supplemented with an HCl modifier) to give 2-[4-(2,2-dimethylpropyl)-2-methyl-phenyl]-1H-quinolin-4-one (338, 16.2 mg, 40%). ESI-MS m/z calc. 305.17798, found 306.5 (M+1)⁺. ¹H NMR (400 MHz, DMSO-d₆) δ (ppm) 11.74 (s, 1H), 8.11 (d, J=8.0 Hz, 1H), 7.68-7.60 (m, 2H), 7.34 (td, J=8.2, 7.8, 2.0 Hz, 2H), 7.17-7.10 (m, 2H), 5.95 (d, J=1.7 Hz, 1H), 2.52 (s, 2H), 2.30 (s, 3H), 0.92 (s, 9H).

### Example 38

### 2-(4-isopropyl-2-methyl-phenyl)-1H-quinolin-4-one (339)

### Step 1: 2-(4-isopropyl-2-methyl-phenyl)-1H-quinolin-4-one (339)

2-(4-isopropyl-2-methyl-phenyl)-1H-quinolin-4-one (339, 5.7 mg, 43%) was prepared using procedure analogous to that found in Example 37, using bromo(isopropyl)zinc instead. ESI-MS m/z calc. 277.15, found 278.5 (M+1)⁺. ¹H NMR (400 MHz, DMSO-d₆) δ (ppm) 11.76 (s, 1H), 8.11 (dd, J=8.0, 1.5 Hz, 1H), 7.65 (ddd, J=8.3, 6.8, 1.5 Hz, 1H), 7.59 (d, J=7.7 Hz, 1H), 7.37-7.31 (m, 2H), 7.30-7.20 (m, 2H), 5.97 (d, J=1.6 Hz, 1H), 2.94 (p, J=6.9 Hz, 1H), 2.29 (s, 3H), 1.24 (d, J=6.9 Hz, 6H) ppm.

### Example 39

### 2-(4-tert-butyl-2,5-dimethyl-phenyl)-1H-1,7-naphthyridin-4-one (340)

### Step 1: 2-(4-tert-butyl-2,5-dimethyl-phenyl)-1H-1,7-naphthyridin-4-one (340)

In a 2 mL microwave vial a solution of 2,4-dichloro-1,7-naphthyridine (30 mg, 0.15 mmol), 2-(4-tert-butyl-2,5-dimethyl-phenyl)-4,4,5,5-tetramethyl-1,3,2-dioxaborolane (44 mg, 0.15 mmol), potassium carbonate (43 mg, 0.31 mmol) and Pd(dppf) (6 mg, 0.007 mmol) in ethanol (1 mL) and water (300 μL) was sparged with nitrogen, sealed and subjected to microwave irradiation at 60° C. for 10 min. The reaction mixture was filtered and purified by preparative reverse phase HPLC (C₁₈) using 20 to 80% ACN in water (supplemented with an HCl modifier) to give 2-(4-tert-butyl-2,5-dimethyl-phenyl)-4-chloro-1,7-naphthyridine (4.9 mg, 10%) ESI-MS m/z calc. 324.14, found 325.3 (M+1)⁺. The obtained intermediate was dissolved in acetic acid (750 μL, 13.19 mmol) and water (0.75 mL) and stirred at 100° C. for 3 h. The reaction mixture was purified by preparative reverse phase HPLC (C₁₈) using 1-99% ACN in water (supplemented with an HCl modifier) to give 2-(4-tert-butyl-2,5-dimethyl-phenyl)-1H-1,7-naphthyridin-4-one (340, 3.4 mg, 7%) ESI-MS m/z calc. 306.17, found 307.5 (M+1)⁺.

### Example 40

### 2-(4-tert-butyl-2-methyl-phenyl)-1H-1,7-naphthyridin-4-one (Hydrochloride salt) (341)

### Step 1: 4-benzyloxy-2-(4-tert-butyl-2-methyl-phenyl)-1,7-naphthyridine (341)

2-(4-tert-butyl-2-methyl-phenyl)-1H-1,7-naphthyridin-4-one (Hydrochloride salt) (341) was prepared from 2,4-dichloro-1,7-naphthyridine and 4-tert-butyl-2-methyl-phenyl)boronic acid using procedure analogous to that found in Example 39. ESI-MS m/z calc. 292.15756, found 293.4 (M+1)⁺. ¹H NMR (400 MHz, DMSO-d₆) δ (ppm) 12.32 (s, 1H), 9.07 (s, 1H), 8.51 (d, J=5.4 Hz, 1H), 7.99 (d, J=5.4 Hz, 1H), 7.44 (s, 1H), 7.40 (d, J=1.2 Hz, 2H), 6.22 (s, 1H), 2.33 (s, 3H), 1.33 (s, 9H).

### Example 41

### 2-[2,5-dimethyl-4-(2,2,2-trifluoro-1,1-dimethyl-ethyl)phenyl]-1H-1,7-naphthyridin-4-one (342)

### Step 1: 2-[2,5-dimethyl-4-(2,2,2-trifluoro-1,1-dimethyl-ethyl)phenyl]-1H-1,7-naphthyridin-4-one (342)

2-[2,5-dimethyl-4-(2,2,2-trifluoro-1,1-dimethyl-ethyl)phenyl]-1H-1,7-naphthyridin-4-one (342) was prepared from 2,4-dichloro-1,7-naphthyridine and Intermediate B-3 using procedure analogous to that found in Example 39. ESI-MS m/z calc. 360.15, found 361.4 (M+1)⁺. ¹H NMR (400 MHz, DMSO-d₆) δ (ppm) 9.04 (s, 1H), 8.49 (d, J=5.3 Hz, 1H), 7.95 (d, J=5.3 Hz, 1H), 7.54 (s, 1H), 7.33 (s, 1H), 6.19 (s, 1H), 2.55 (s, 3H), 2.31 (s, 3H), 1.72 (s, 6H).

### Example 42

### 2-(4-tert-butyl-5-chloro-2-methyl-phenyl)-4-oxo-1H-quinoline-5-carboxamide (343)

### Step 1: 2-(4-tert-butyl-5-chloro-2-methyl-phenyl)-4-oxo-1H-quinoline-5-carboxamide (343)

4-benzyloxy-2-chloro-quinoline-5-carbonitrile (100 mg, 0.34 mmol), 2-(4-tert-butyl-5-chloro-2-methyl-phenyl)-4,4,5,5-tetramethyl-1,3,2-dioxaborolane (105 mg, 0.34 mmol) and aqueous potassium phosphate (850 μL of 1 M, 0.85 mmol) were combined in dioxane (2 mL) and purged with nitrogen for 10 minutes. PdCl₂(dtbpf) (22 mg, 0.03 mmol) was added and the reaction was purged with nitrogen for an additional 5 min, then sealed and stirred at room temperature for 1 hour. The reaction mixture was partitioned between ethyl acetate and water. The organics were separated, washed with brine, dried over magnesium sulfate and evaporated. The crude material was purified via silica gel column chromatography using 0 to 20% ethyl acetate in hexanes to give 4-benzyloxy-2-(4-tert-butyl-5-chloro-2-methyl-phenyl)quinoline-5-carbonitrile (60 mg, 40%) as a white solid. ESI-MS m/z calc. 440.16, found 441.5 (M+1)⁺. It was taken up in toluene (1 mL) and TFA (600 μL, 7.78 mmol) was added. The resulting reaction mixture was heated at 60° C. for 15 h. The solvent was evaporated and the crude material was purified via silica gel column chromatography using 0 to 80% DCM in ethyl acetate, to obtain 2-(4-tert-butyl-5-chloro-2-methyl-phenyl)-4-oxo-1H-quinoline-5-carboxamide (343, 31 mg, 25%) ESI-MS m/z calc. 368.13, found 369.4 (M+1)⁺. ¹H NMR (400 MHz, DMSO-d₆) δ (ppm) 11.76 (s, 1H), 7.60 (dt, J=17.7, 8.2 Hz, 2H), 7.49 (s, 1H), 7.46 (s, 1H), 7.41-7.31 (m, 1H), 7.15 (s, 1H), 7.10 (d, J=6.8 Hz, 1H), 5.99 (d, J=1.7 Hz, 1H), 2.31 (s, 3H), 1.49 (s, 9H).

### Example 43

### 2-(4-tert-butyl-2-methyl-phenyl)-4-oxo-1H-quinoline-5-carboxamide (344)

### Step 1: 2-(4-tert-butyl-2-methyl-phenyl)-4-oxo-1H-quinoline-5-carboxamide (344)

2-(4-tert-butyl-2-methyl-phenyl)-4-oxo-1H-quinoline-5-carboxamide (344) was prepared from Intermediate A-4 and (4-(tert-butyl)-2-methylphenyl)boronic acid using a procedure analogous to that found in Example 42. ESI-MS m/z calc. 334.17, found 335.6 (M+1)⁺. ¹H NMR (400 MHz, DMSO-d₆) δ (ppm) 11.70 (s, 1H), 7.63-7.53 (m, 2H), 7.44-7.30 (m, 4H), 7.17-7.04 (m, 2H), 5.93 (s, 1H), 2.32 (s, 3H), 1.33 (s, 9H).

### Example 44

### 2-(4-tert-butyl-2-methyl-phenyl)-5,7-difluoro-1H-quinolin-4-one (345)

### Step 1: 4-chloro-5,7-difluoro-1-oxido-quinolin-1-ium

To a flask charged with 4-chloro-5,7-difluoro-quinoline (2 g, 10 mmol) in DCM (20 mL), 3-chlorobenzenecarboperoxoic acid (2.97 g, 12.05 mmol) was added and stirred for 16 hours at ambient temperature. The reaction mixture was quenched with saturated sodium bicarbonate solution. The aqueous layer was extracted with DCM (3×), dried over magnesium sulfate, filtered and concentrated under reduced pressure to give 4-chloro-5,7-difluoro-1-oxido-quinolin-1-ium (2.11 g, 98%) ESI-MS m/z calc. 214.99, found 216.1 (M+1)⁺.

### Step 2: 2-bromo-4-chloro-5,7-difluoro-quinoline

To a solution of 4-chloro-5,7-difluoro-1-oxido-quinolin-1-ium (2.11 g, 9.79 mmol) in DCM (30 mL) was added phosphoryl tribromide (2.9 g, 10.12 mmol) at room temperature and stirred for 16 h. The reaction mixture was poured onto ice cold water and neutralized with potassium carbonate. The aqueous layer was extracted with DCM (2×), dried over magnesium sulfate, filtered and concentrated. The crude product was purified via silica gel column chromatography using 0 to 10% ethyl acetate in hexanes to obtain 2-bromo-4-chloro-5,7-difluoro-quinoline (1.79 g, 66%). ESI-MS m/z calc. 276.91, found 280.0 (M+3)⁺.

### Step 3: 2-(4-tert-butyl-2-methyl-phenyl)-4-chloro-5,7-difluoro-quinoline

A microwave vial charged with 2-bromo-4-chloro-5,7-difluoro-quinoline (150 mg, 0.54 mmol), 2-(4-tert-butyl-2-methyl-phenyl)-4,4,5,5-tetramethyl-1,3,2-dioxaborolane (145 mg, 0.53 mmol), Pd(PPh₃)₄(10 mg, 0.009 mmol), Cs₂CO₃ (525 mg, 1.61 mmol), dioxane (3 mL) and water (300 μL) was degassed by bubbling nitrogen for 30-60 seconds. The vial was capped and heated at 75° C. for 16 hours. The reaction mixture was filtered, and the solvent was evaporated. The crude material was purified via silica gel column chromatography using hexanes to obtain 2-(4-tert-butyl-2-methyl-phenyl)-4-chloro-5,7-difluoro-quinoline (144 mg, 77%) ESI-MS m/z calc. 345.11, found 346.5 (M+1)⁺.

### Step 4: 2-(4-tert-butyl-2-methyl-phenyl)-5,7-difluoro-1H-quinolin-4-one (345)

A solution of 2-(4-tert-butyl-2-methyl-phenyl)-4-chloro-5,7-difluoro-quinoline (40 mg, 0.12 mmol) was taken up in 0.25 mL of (4:1) mixture acetic acid (200 μL) and water (50 μL). The reaction mixture was heated at 120° C. for 90 minutes. The solvent was evaporated, and the crude material was neutralized with few drops of saturated sodium bicarbonate solution. The aqueous layer was extracted with ethyl acetate. The organic layer was dried over magnesium sulfate, filtered and concentrated. The crude material was purified via silica gel column chromatography using 0 to 50% ethyl acetate in hexanes to obtain 2-(4-tert-butyl-2-methyl-phenyl)-5,7-difluoro-1H-quinolin-4-one (345, 8 mg, 20%). ESI-MS m/z calc. 327.14, found 328.4 (M+1)⁺. ¹H NMR (400 MHz, DMSO-d₆) δ (ppm) 11.83 (s, 1H), 7.42 (d, J=1.8 Hz, 1H), 7.40-7.31 (m, 2H), 7.09 (tdd, J=12.0, 9.6, 2.6 Hz, 2H), 5.91 (s, 1H), 2.31 (s, 3H), 1.32 (s, 9H).

### Example 45

### 2-[4-tert-butyl-2-(3-hydroxypropyl)phenyl]-1H-1,6-naphthyridin-4-one (346) and 3-bromo-2-[4-tert-butyl-2-(3-hydroxypropyl)phenyl]-1H-1,6-naphthyridin-4-one (347)

### Step 1: [(E)-3-[2-(4-benzyloxy-1,6-naphthyridin-2-yl)-5-tert-butyl-phenyl]allyloxy]-tert-butyl-dimethyl-silane

A solution of 4-benzyloxy-2-(4-tert-butyl-2-chloro-phenyl)-1,6-naphthyridine (500 mg, 1.24 mmol), tert-butyl-dimethyl-[(E)-3-(4,4,5,5-tetramethyl-1,3,2-dioxaborolan-2-yl)allyloxy]silane (407 mg, 1.36 mmol), and potassium carbonate (951 mg, 6.88 mmol) in DMF (3 mL) was flushed with nitrogen for 1 minute and then Pd(dppf)₂Cl₂·DCM (51 mg, 0.06 mmol) was added. The resulting mixture was sparged with nitrogen (bubbling via syringe needle) for 10 minutes, capped and stirred at 75° C. for 3 h. The reaction mixture was cooled to room temperature and directly purified by silica gel column chromatography using a gradient eluent of 1 to 100% ethyl acetate in hexanes to give [(E)-3-[2-(4-benzyloxy-1,6-naphthyridin-2-yl)-5-tert-butyl-phenyl]allyloxy]-tert-butyl-dimethyl-silane (571 mg, 85%) as an off white solid. ESI-MS m/z calc. 538.3016, found 539.8 (M+1)⁺. ¹H NMR (400 MHz, CD₃OD) δ (ppm) 9.65 (q, J=0.9 Hz, 1H), 8.80 (dd, J=6.0, 1.5 Hz, 1H), 7.96 (dt, J=5.9, 1.1 Hz, 1H), 7.82 (d, J=1.9 Hz, 1H), 7.64 (dt, J=8.0, 1.7 Hz, 2H), 7.60-7.43 (m, 5H), 7.36 (d, J=2.5 Hz, 1H), 6.75 (dt, J=15.6, 2.1 Hz, 1H), 6.39 (dt, J=15.6, 4.2 Hz, 1H), 5.53 (d, J=3.0 Hz, 2H), 4.36 (dt, J=2.9, 1.4 Hz, 2H), 1.49 (d, J=1.0 Hz, 9H), 0.71 (s, 9H), −0.00 (s, 6H).

### Step 2: 2-[4-tert-butyl-2-(3-hydroxypropyl)phenyl]-1H-1,6-naphthyridin-4-one (346)

A reaction vial was loaded with [(E)-3-[2-(4-benzyloxy-1,6-naphthyridin-2-yl)-5-tert-butyl-phenyl]allyloxy]-tert-butyl-dimethyl-silane (1.0 g, 1.86 mmol), 10% Pd/C (200 mg, 0.19 mmol), and septa capped. The vial was purged with nitrogen and to the reaction was added ethanol (10 mL) via syringe. The reaction mixture was purged with molecular hydrogen and then stirred vigorously while under 1 atmosphere of hydrogen (balloon) for 19 hours. The reaction mixture was purged with nitrogen and filtered. To the filtrate, was added concentrated hydrochloric acid (310 μL of 12 M, 3.720 mmol) and the solution was then concentrated under reduced pressure. The residue was diluted with methanol (3 mL), filtered, and purified by reverse phase preparative chromatography (C₁₈) column using 1 to 99% acetonitrile in water containing 5 mM hydrochloric acid to give 2-[4-tert-butyl-2-(3-hydroxypropyl)phenyl]-1H-1,6-naphthyridin-4-one (346, 210 mg, 33%) as a clear oil. ESI-MS m/z calc. 336.18378, found 337.3 (M+1)⁺. ¹H NMR (400 MHz, CD₃OD) δ (ppm) 9.47 (s, 1H), 8.71 (dd, J=6.9, 1.1 Hz, 1H), 7.92 (d, J=6.9 Hz, 1H), 7.52 (d, J=2.0 Hz, 1H), 7.46 (dd, J=8.1, 1.9 Hz, 1H), 7.39 (d, J=8.1 Hz, 1H), 6.50 (s, 1H), 3.51 (t, J=6.1 Hz, 2H), 2.94-2.70 (m, 2H), 1.88-1.70 (m, 2H), 1.37 (s, 9H).

### Step 3: 3-bromo-2-[4-tert-butyl-2-(3-hydroxypropyl)phenyl]-1H-1,6-naphthyridin-4-one (347)

To a stirring solution of 2-[4-tert-butyl-2-(3-hydroxypropyl)phenyl]-1H-1,6-naphthyridin-4-one (346, 165 mg, 0.49 mmol) in DCM (1.7 mL) at ambient temperature was added N-bromosuccinimide (87 mg, 0.49 mmol) in a single portion. The reaction mixture was stirred for 15 minutes and the reaction mixture was directly purified by reverse phase preparative chromatography column (C₁₈) using 1 to 99% acetonitrile in water containing 5 mM hydrochloric acid to give 3-bromo-2-[4-tert-butyl-2-(3-hydroxypropyl)phenyl]-1H-1,6-naphthyridin-4-one (347, 5.1 mg, 2%) as a white solid. ESI-MS m/z calc. 414.09, found 417.2 (M+3)⁺. ¹H NMR (400 MHz, CD₃OD) δ (ppm) 9.56 (s, 1H), 8.85-8.61 (m, 1H), 7.86 (td, J=7.2, 2.4 Hz, 1H), 7.55 (d, J=1.9 Hz, 1H), 7.49 (dd, J=8.1, 2.0 Hz, 1H), 7.32 (d, J=8.2 Hz, 1H), 3.50 (t, J=6.2 Hz, 2H), 2.68 (ddd, J=9.1, 7.0, 2.1 Hz, 2H), 1.78 (tdq, J=13.4, 8.8, 6.8 Hz, 2H), 1.39 (s, 9H).

### Example 46

### 2-(5-tert-butyl-3,6-dimethyl-pyrazin-2-yl)-4-oxo-1H-1,6-naphthyridine-5-carboxamide (Hydrochloride salt) (246)

### Step 1: 5-tert-butyl-3,6-dichloro-pyrazine-2-carbonitrile

2,2-dimethylpropanoic acid (3.11 g, 3.5 mL, 30.46 mmol) and ammonium persulfate (6.8 g, 29.8 mmol) were added to a suspension of 3,6-dichloropyrazine-2-carbonitrile (5 g, 28.74 mmol) in water (100 mL). Silver nitrate (7.5 g, 44.15 mmol) was added and reaction mixture was stirred at 80° C. for 18 hours. The reaction mixture was cooled to room temperature and extracted using ethyl acetate (3×150 mL). The combined organic layer was washed with brine (200 mL), dried over sodium sulfate and concentrated under reduced pressure. The residue was purified by silica gel column chromatography using 0% to 10% of ethyl acetate in heptanes to afford 5-tert-butyl-3,6-dichloro-pyrazine-2-carbonitrile (492 mg, 7%) as a white solid ¹H NMR (400 MHz, CDCl₃) δ (ppm) 1.55 (s, 9H).

### Step 2: 5-tert-butyl-3,6-dimethyl-pyrazine-2-carbonitrile

A solution of 5-tert-butyl-3,6-dichloro-pyrazine-2-carbonitrile (555 mg, 2.41 mmol), methylboronic acid (730 mg, 12.19 mmol) and potassium carbonate (2 g, 14.47 mmol) in dioxane (10 mL) was bubbled with nitrogen for 5 minutes in a sealed tube. Pd(PPh₃)₄(280 mg, 0.24 mmol) was added and the reaction mixture was bubbled with nitrogen for 2 more minutes. The tube was sealed, and reaction mixture was stirred at 120° C. overnight. The reaction mixture was cooled to room temperature and diluted with ethyl acetate. The reaction mixture was concentrated under reduced pressure and purified by silica gel chromatography using 0% to 15% of ethyl acetate in heptanes to afford 5-tert-butyl-3,6-dimethyl-pyrazine-2-carbonitrile (304 mg, 67%) as a light yellowish oil. ESI-MS m/z calc. 189.13, found 190.0 (M+1)⁺. ¹H NMR (400 MHz, CDCl₃) δ (ppm) 2.75 (s, 3H), 2.71 (s, 3H), 1.45 (s, 9H).

### Step 3: 5-tert-butyl-3,6-dimethyl-pyrazine-2-carboxylic acid

To a solution of 5-tert-butyl-3,6-dimethyl-pyrazine-2-carbonitrile (486 mg, 2.46 mmol) in ethanol (5 mL) was added an aqueous solution of NaOH (2.5 mL of 20 M, 50 mmol). The mixture was stirred at 120° C. for 2 hours. The reaction mixture was cooled to room temperature and ethanol was removed under reduced pressure and an aqueous solution of 6 M HCl was added (pH ˜1-2). The aqueous mixture was diluted with water (10 mL) and extracted using ethyl acetate (3×10 mL). The combined organic layer was dried over sodium sulfate, filtered and concentrated under reduced pressure to afford crude 5-tert-butyl-3,6-dimethyl-pyrazine-2-carboxylic acid (500 mg, 97%) as a beige solid. ESI-MS m/z calc. 208.12, found 209.2 (M+1)⁺. ¹H NMR (400 MHz, CDCl₃) δ (ppm) 2.93 (s, 3H), 2.78 (s, 3H), 1.48 (s, 9H).

### Step 4: N-(3-acetyl-4-pyridyl)-5-tert-butyl-3,6-dimethyl-pyrazine-2-carboxamide

T3P (in ethyl acetate) (2.1 mL of 50% w/v, 3.3 mmol) was added to a solution of 5-tert-butyl-3,6-dimethyl-pyrazine-2-carboxylic acid (219 mg, 1.05 mmol), 1-(4-amino-3-pyridyl)ethanone (Hydrochloric Acid (1)) (290 mg, 1.68 mmol) and triethylamine (653 mg, 0.9 mL, 6.46 mmol) in ethyl acetate (3 mL) and the reaction mixture was stirred at room temperature overnight. The reaction mixture was quenched with saturated aqueous sodium bicarbonate solution (20 mL) and the aqueous layer was extracted using ethyl acetate (3×30 mL). The combined organic layer was dried over sodium sulfate and concentrated under reduced pressure. The residue was purified by silica gel chromatography using 0% to 100% of ethyl acetate in heptanes to afford N-(3-acetyl-4-pyridyl)-5-tert-butyl-3,6-dimethyl-pyrazine-2-carboxamide (149 mg, 43%) as a yellow solid. ESI-MS m/z calc. 326.17, found 327.2 (M+1)⁺. ¹H NMR (400 MHz, CDCl₃) δ (ppm) 13.70 (br. s, 1H), 9.17 (s, 1H), 8.91 (d, J=5.9 Hz, 1H), 8.68 (d, J=5.9 Hz, 1H), 2.95 (s, 3H), 2.94 (s, 3H), 2.78 (s, 3H), 1.50 (s, 9H).

### Step 5: 2-(5-tert-butyl-3,6-dimethyl-pyrazin-2-yl)-1H-1,6-naphthyridin-4-one

In a sealed tube, potassium tert-butoxide (113 mg, 1.01 mmol) was added to a solution of N-(3-acetyl-4-pyridyl)-5-tert-butyl-3,6-dimethyl-pyrazine-2-carboxamide (150 mg, 0.46 mmol) in dioxane (3 mL). The solution was bubbled with nitrogen, the tube was sealed, and the reaction mixture was heated at 50° C. for 1 hour then 3 hours at 100° C. The reaction mixture was cooled room temperature and diluted with saturated aqueous ammonium chloride (10 mL) and extracted using DCM (3×15 mL). The combined organic layer was dried over sodium sulfate and concentrated under reduced pressure to afford 2-(5-tert-butyl-3,6-dimethyl-pyrazin-2-yl)-1H-1,6-naphthyridin-4-one (131 mg, 93%) as an orange solid. ESI-MS m/z calc. 326.17, found 327.2 (M+1)⁺. ¹H NMR (400 MHz, CDCl₃) δ (ppm) 10.04 (br. s, 1H), 9.56 (s, 1H), 8.70 (d, J=5.9 Hz, 1H), 7.34 (d, J=5.6 Hz, 1H), 6.99 (d, J=1.2 Hz, 1H), 2.87 (s, 3H), 2.85 (s, 3H), 1.50 (s, 9H).

### Step 6: 2-(5-tert-butyl-3,6-dimethyl-pyrazin-2-yl)-6-oxido-1H-1,6-naphthyridin-6-ium-4-one

mCPBA (160 mg, 0.71 mmol) was added to a solution of 2-(5-tert-butyl-3,6-dimethyl-pyrazin-2-yl)-1H-1,6-naphthyridin-4-one (110 mg, 0.36 mmol) in DCM (5 mL) and reaction mixture was stirred at room temperature for 4 hours. The reaction mixture was diluted with ethyl acetate (20 mL) and extracted using saturated aqueous sodium bicarbonate solution (3×30 mL). The aqueous layer was acidified using 3N HCl until pH 3-4 and extracted using ethyl acetate (3×30 mL). The combined organic layer was dried over sodium sulfate and concentrated under reduced pressure. The residue was purified by reverse phase chromatography using 5 to 95% of acetonitrile in water (0.1% formic acid) to afford 2-(5-tert-butyl-3,6-dimethyl-pyrazin-2-yl)-6-oxido-1H-1,6-naphthyridin-6-ium-4-one (24 mg, 20%) as a yellow solid. ESI-MS m/z calc. 324.16, found 325.4 (M+1)⁺. ¹H NMR (400 MHz, CDCl₃) δ (ppm) 10.26 (br. s, 1H), 9.08 (s, 1H), 8.35 (dd, J=7.3, 2.0 Hz, 1H), 7.42 (d, J=7.1 Hz, 1H), 6.96 (s, 1H), 2.87 (s, 3H), 2.84 (s, 3H), 1.50 (s, 9H).

### Step 7: 2-(5-tert-butyl-3,6-dimethyl-pyrazin-2-yl)-4-oxo-1H-1,6-naphthyridine-5-carbonitrile

In a sealed tube, TMSCN (79.300 mg, 0.1 mL, 0.7993 mmol) and triethylamine (108.90 mg, 0.15 mL, 1.0762 mmol) were added to a solution of 2-(5-tert-butyl-3,6-dimethyl-pyrazin-2-yl)-6-oxido-1H-1,6-naphthyridin-6-ium-4-one (24 mg, 0.07 mmol) in DCM (2 mL) under nitrogen atmosphere. The tube was sealed, and reaction mixture was stirred at room temperature overnight. The reaction mixture was diluted with saturated sodium bicarbonate (20 mL) and extracted using ethyl acetate (3×15 mL). The combined organic layer was dried over sodium sulfate and concentrated under reduced pressure to afford 2-(5-tert-butyl-3,6-dimethyl-pyrazin-2-yl)-4-oxo-1H-1,6-naphthyridine-5-carbonitrile (21 mg, 85%) as a beige solid. ESI-MS m/z calc. 333.159, found 334.4 (M+1)⁺. ¹H NMR (400 MHz, CDCl₃) δ (ppm) 10.04 (br. s, 1H), 8.56 (d, J=5.9 Hz, 1H), 7.35 (d, J=5.6 Hz, 1H), 6.86 (s, 1H), 2.69 (s, 3H), 2.67 (s, 3H), 1.32 (s, 9H).

### Step 8: 2-(5-tert-butyl-3,6-dimethyl-pyrazin-2-yl)-4-oxo-1H-1,6-naphthyridine-5-carboxamide (246)

In a sealed tube, to a solution of 2-(5-tert-butyl-3,6-dimethyl-pyrazin-2-yl)-4-oxo-1H-1,6-naphthyridine-5-carbonitrile (21 mg, 0.0630 mmol) in toluene (2 mL) was added trifluoroacetic acid (740 mg, 0.5 mL, 6.49 mmol) and a drop of water. The tube was sealed, and the reaction mixture stirred at 70° C. for 24 hours, then at 75° C. for an additional 24 hours. The reaction mixture was evaporated to dryness under reduced pressure and was purified by reversed-phase flash chromatography (C₁₈) using 5 to 95% acetonitrile in water supplemented with 0.1% formic acid, followed by a second purification using SFC separation by the following conditions: a Lux Column, i-Amylose 3 (250×21.2 mm), 5 μM column at 40° C., eluent: 30% methanol, 70% CO₂, flow rate: 75 mL/min, concentration: 5 mg/mL in methanol (no modifier), injection volume: 500 μL, pressure: 100 bar, wavelength: 220 nm. to afford 2-(5-tert-butyl-3,6-dimethyl-pyrazin-2-yl)-4-oxo-1H-1,6-naphthyridine-5-carboxamide (Hydrochloride salt) (246, 8 mg, 36%) as a white solid. ESI-MS m/z calc. 351.17, found 352.2 (M+1)⁺. ¹H NMR (400 MHz, CDCl₃) δ (ppm) 15.40 (br. s, 1H), 8.99 (br. s, 1H), 8.66 (d, J=5.6 Hz, 1H), 8.17 (d, J=5.4 Hz, 1H), 7.69 (s, 1H), 6.13 (br. s, 1H), 2.81 (s, 3H), 2.79 (s, 3H), 1.50 (s, 9H).

### Example 47

### 3-bromo-2-(4-tert-butyl-2-methyl-phenyl)-4-oxo-1H-1,6-naphthyridine-5-carboxamide (189)

### Step 1: 3-bromo-2-(4-tert-butyl-2-methyl-phenyl)-4-oxo-1H-1,6-naphthyridine-5-carboxamide (189)

To 2-(4-tert-butyl-2-methyl-phenyl)-4-oxo-1H-1,6-naphthyridine-5-carboxamide (10 mg, 0.03 mmol) (211) in DCM (300 μL) and acetic acid (50 μL) at 0° C. was added NBS (6 mg, 0.03 mmol) and the resulting mixture allowed to warm to room temperature and stirred for 5 minutes. The solvent was evaporated, and the crude material was purified via silica gel column chromatography using 0 to 80% ethyl acetate in DCM to obtain 3-bromo-2-(4-tert-butyl-2-methyl-phenyl)-4-oxo-1H-1,6-naphthyridine-5-carboxamide (189, 6.5 mg, 52%). ESI-MS m/z calc. 413.07, found 416.2 (M+3)⁺. ¹H NMR (400 MHz, DMSO-d₆) δ (ppm) 12.50 (s, 1H), 8.55 (d, J=5.8 Hz, 1H), 7.57 (s, 1H), 7.48-7.39 (m, 3H), 7.36 (s, 1H), 7.31 (d, J=8.0 Hz, 1H), 2.20 (s, 3H), 1.34 (s, 9H).

### Example 48

### 2-(4-tert-butyl-2-methyl-phenyl)-3-methoxy-4-oxo-1H-1,6-naphthyridine-5-carboxamide (348)

### Step 1: 3-bromo-2-(4-tert-butyl-2-methyl-phenyl)-4-oxo-1H-1,6-naphthyridine-5-carbonitrile

To a solution of crude 2-(4-tert-butyl-2-methyl-phenyl)-4-oxo-1H-1,6-naphthyridine-5-carbonitrile (250 mg) in DCM (12 mL) and AcOH (4 mL) was added N-bromosuccinimide (270 mg, 1.5 mmol) in a single portion and the reaction mixture was stirred at room temperature for 1 h. The reaction mixture was concentrated and purified by silica gel column chromatography using 0 to 100% ethyl acetate in hexanes followed by a second purification via reverse phase preparative chromatography (C₁₈) using 1 to 99% ACN in water (supplemented with an HCl modifier) to give 3-bromo-2-(4-tert-butyl-2-methyl-phenyl)-4-oxo-1H-1,6-naphthyridine-5-carbonitrile (225 mg, 38%); ESI-MS m/z calc. 395.06, found 396.4 (M+1)⁺. ¹H NMR (400 MHz, CD₃OD) δ (ppm) 8.72 (d, J=5.9 Hz, 1H), 7.65 (d, J=5.9 Hz, 1H), 7.50-7.40 (m, 2H), 7.29 (d, J=8.0 Hz, 1H), 2.26 (s, 3H), 1.37 (s, 9H).

### Step 2: 2-(4-tert-butyl-2-methyl-phenyl)-3-methoxy-4-oxo-1H-1,6-naphthyridine-5-carboxamide (348)

3-bromo-2-(4-tert-butyl-2-methyl-phenyl)-4-oxo-1H-1,6-naphthyridine-5-carbonitrile (60 mg, 0.15 mmol) and CuI (32 mg, 0.17 mmol) were suspended in cyclopentyl methyl ether (600 μL). The solution was sparged with nitrogen for 5 minutes and sodium methoxide (25 wt % in methanol) (140 μL of 25% w/w, 0.61 mmol) was added via syringe. The reaction mixture was stirred at 70° C. for 18 h. The reaction mixture was cooled to room temperature, filtered, washed with ethyl acetate, concentrated, and resuspended in toluene (6 mL). Trifluoroacetic acid (3 mL, 38.94 mmol) was added dropwise, and the reaction mixture was stirred at 65° C. for 2 h. The reaction mixture was concentrated and purified by silica gel column chromatography using 0-15% MeOH in DCM followed by a second purification using supercritical fluid chromatography (SFC): [ChiralPak IG (21.2×250 mm, 5 um), 40° C., isocratic, 18% MeOH+20 mM NH₃, Flow: 70 mL/min, Pressure 148 bar] to afford 2-(4-tert-butyl-2-methyl-phenyl)-3-methoxy-4-oxo-1H-1,6-naphthyridine-5-carboxamide (348, 33 mg, 59%) as a light yellow solid. ESI-MS m/z calc. 365.17, found 366.1 (M+1)⁺. ¹H NMR (400 MHz, CD₃OD) δ (ppm) 8.47 (d, J=5.9 Hz, 1H), 7.51-7.39 (m, 3H), 7.34 (d, J=8.0 Hz, 1H), 3.60 (s, 3H), 2.27 (s, 3H), 1.37 (s, 9H).

### Example 49

### 2-(4-tert-butyl-2-methyl-phenyl)-3-ethoxy-4-oxo-1H-1,6-naphthyridine-5-carboxamide (349)

### Step 1: 2-(4-tert-butyl-2-methyl-phenyl)-3-ethoxy-4-oxo-1H-1,6-naphthyridine-5-carboxamide (349)

2-(4-tert-butyl-2-methyl-phenyl)-3-ethoxy-4-oxo-1H-1,6-naphthyridine-5-carboxamide (349) was prepared using a procedure analogous to that found in Example 48, using sodium ethoxide in place of sodium methoxide. ESI-MS m/z calc. 379.1896, found 380.1 (M+1)⁺. ¹H NMR (400 MHz, CD₃OD) δ (ppm) 8.46 (d, J=6.0 Hz, 1H), 7.52-7.37 (m, 3H), 7.34 (d, J=8.0 Hz, 1H), 4.05-3.68 (m, 2H), 2.28 (s, 3H), 1.37 (s, 9H), 1.00 (t, J=7.0 Hz, 3H).

### Example 50

### 2-(4-tert-butyl-2-methyl-phenyl)-5-hydroxy-1H-quinolin-4-one (350)

### Step 1: 2-(4-tert-butyl-2-methyl-phenyl)-5-methoxy-1H-quinolin-4-one (350)

A microwave vial charged with 4-benzyloxy-2-chloro-5-methoxy-quinoline (100 mg, 0.33 mmol), K₂CO₃ (95 mg, 0.69 mmol), 2-(4-tert-butyl-2-methyl-phenyl)-4,4,5,5-tetramethyl-1,3,2-dioxaborolane (137 mg, 0.5 mmol) and SPhos Pd G3 (26 mg, 0.03 mmol) in ethanol (2 mL) and water (1 mL) was sparged with nitrogen, sealed and subjected to microwave irradiation at 120° C. for 20 minutes. The mixture was filtered and purified by preparative reverse phase HPLC (C₁₈) using 20-80% ACN in water (HCl modifier) to give 2-(4-tert-butyl-2-methyl-phenyl)-5-methoxy-1H-quinolin-4-one (20.4 mg, 19%). ESI-MS m/z calc. 321.17, found 322.6 (M+1)⁺.

The obtained material was dissolved in DCM (2 mL) and treated with tribromoborane (150 μL of 1 M, 0.15 mmol) and stirred at room temperature for 3 h. The mixture was quenched with methanol, evaporated and purified by preparative reverse phase HPLC (C₁₈) using 1-99% ACN in water (HCl modifier) to obtain 2-(4-tert-butyl-2-methyl-phenyl)-5-hydroxy-1H-quinolin-4-one (350, 10.9 mg, 10%) ESI-MS m/z calc. 307.15, found 308.4 (M+1)⁺. ¹H NMR (400 MHz, CDCl₃) δ (ppm) 14.08 (s, 1H), 8.24 (s, 1H), 7.46 (t, J=8.2 Hz, 1H), 7.36-7.28 (m, 3H), 6.70 (dd, J=15.7, 7.8 Hz, 2H), 6.19 (d, J=1.7 Hz, 1H), 2.38 (s, 3H), 1.35 (s, 9H).

### Example 51

### Preparation of Compounds 351, 352, 353, 354, and 355

### Step 1: 4-benzyloxy-2-(2-fluoro-3-quinolyl)-1,6-naphthyridine-5-carbonitrile

4-benzyloxy-2-chloro-1,6-naphthyridine-5-carbonitrile (2.54 g, 8.59 mmol), (2-fluoro-3-quinolyl)boronic acid (2.59 g, 10.17 mmol), and potassium carbonate (3.51 g, 25.40 mmol) were suspended in dioxane (50 mL) and water (5 mL). The mixture was sparged with nitrogen for 10 min. Pd(dppf)₂Cl₂·DCM (784.3 mg, 0.96 mmol) was added, and the reaction mixture was sparged with nitrogen for 10 minutes at room temperature, then stirred at 60° C. for 70 min. The reaction mixture was diluted with ethyl acetate and water. The two layers were separated, and the aqueous layer was extracted with ethyl acetate (3×). Combined organic layer was washed with brime, dried over sodium sulfate, filtered and the solvent was removed under reduced pressure. Purification by silica gel chromatography using 0 to 20% ethyl acetate in hexanes provided 4-benzyloxy-2-(2-fluoro-3-quinolyl)-1,6-naphthyridine-5-carbonitrile (2.74 g, 78%). ESI-MS m/z calc. 406.12, found 407.2 (M+1)⁺.

### Step 2

General Procedure: 4-benzyloxy-2-(2-fluoro-3-quinolyl)-1,6-naphthyridine-5-carbonitrile (1 eq), Cs₂CO₃ (6 eq) and amine (3 eq) were combined in acetonitrile (20 vol eq) and stirred at 80° C. for 2.5 h. The reaction was cooled down to room temperature, filtered and the solvent was evaporated under reduced pressure. The resulting crude material was taken up in toluene (120 vol eq) and TFA (120 vol eq) and was stirred at 65° C. in a 40 mL vial (vial exposed to air) for 16 h. The solvent was evaporated under reduced pressure. The residue was dissolved in DMSO, filtered and purified by reverse phase HPLC (10-99% acetonitrile/5 mM HCl over 15 min) to yield the desired products listed in Table 5, using the appropriate commercially available amines.

TABLE 5

| | | | LC/MS | |
|---|---|---|---|---|
| | | | (m/z | |
| | | | calc.) | |
| Cmpd. | | | Found | |
| No. | Compound Name | Amine | M + 1 | NMR (shifts in ppm) |
| 351 | 2-[2-(diisobutylamino)-3- | | 443.54 | |
| | quinolyl]-4-oxo-1H-1,6- | | 444.4 | |
| | naphthyridine-5-carboxamide | | | |
| 352 | 2-[2-(dipropylamino)-3- quinolyl]-4-oxo-1H-1,6- naphthyridine-5-carboxamide | | 415.49 416.3 | |
| 353 | 2-[2-[butyl(ethyl)amino]-3- quinolyl]-4-oxo-1H-1,6- naphthyridine-5-carboxamide | | 415.49 416.3 | |
| 354 | 2-[2-[butyl(methyl)amino]-3- quinolyl]-4-oxo-1H-1,6- naphthyridine-5-carboxamide | | 401.46 402.3 | |
| 355 | 2-[2-(dimethylamino)-3- quinolyl]-4-oxo-1H-1,6- | | 359.38 360.2 | ¹H NMR (400 MHz, CD₃OD) δ (ppm) 8.78 (s, 1H), 8.75 (d, J = |
| | naphthyridine-5-carboxamide | | | 6.0 Hz, 1H), 8.09 (d, J = 8.5 Hz, |
| | | | | 1H), 8.04 (d, J = 8.0 Hz, 1H), |
| | | | | 8.00-7.92 (m, 1H), 7.87 (d, J = |
| | | | | 6.0 Hz, 1H), 7.65 (t, J = 7.6 Hz, |
| | | | | 1H), 6.94 (s, 1H), 3.27 (s, 6H). |

### Example 52

### 2-[5-chloro-4-(2-hydroxy-1,1-dimethyl-ethyl)-2-methyl-phenyl]-4-oxo-1H-1,6-naphthyridine-5-carboxamide (356)

### Step 1: 4-benzyloxy-2-[5-chloro-4-(2-hydroxy-1,1-dimethyl-ethyl)-2-methyl-phenyl]-1,6-naphthyridine-5-carbonitrile

4-Benzyloxy-2-chloro-1,6-naphthyridine-5-carbonitrile (75 mg, 0.25 mmol), 2-[2-chloro-5-methyl-4-(4,4,5,5-tetramethyl-1,3,2-dioxaborolan-2-yl)phenyl]-2-methyl-propan-1-ol (83 mg, 0.25 mmol), and aqueous potassium phosphate (640 μL of 1 M, 0.64 mmol) were combined in dioxane (1.5 mL) and purged with nitrogen for 1 min PdCl₂(dtbpf) (33 mg, 0.05 mmol) was added and the reaction was purged with nitrogen for an additional 5 min, then sealed and stirred at room temperature for 1 h. The mixture was partitioned between ethyl acetate and water. The organic layer was separated, washed with brine, dried over magnesium sulfate, filtered and concentrated. Purification by silica gel chromatography using 0 to 80% ethyl acetate in hexanes provided 4-benzyloxy-2-[5-chloro-4-(2-hydroxy-1,1-dimethyl-ethyl)-2-methyl-phenyl]-1,6-naphthyridine-5-carbonitrile (90 mg, 78%). ESI-MS m/z calc. 457.15, found 458.4 (M+1)⁺.

### Step 2: 2-[5-chloro-4-(2-hydroxy-1,1-dimethyl-ethyl)-2-methyl-phenyl]-4-oxo-1H-1,6-naphthyridine-5-carboxamide (356)

4-Benzyloxy-2-[5-chloro-4-(2-hydroxy-1,1-dimethyl-ethyl)-2-methyl-phenyl]-1,6-naphthyridine-5-carbonitrile (90 mg, 0.197 mmol), H₂O₂(145 μL of 30% w/v, 1.28 mmol) and aqueous KOH (140 μL of 40% w/v, 1 mmol) were combined in methanol (1 mL) and the reaction mixture was stirred at room temperature overnight. The reaction mixture was quenched with water and the aqueous layer was extracted with ethyl acetate (3×). The combined organic layer was dried over magnesium sulfate, filtered and concentrated. The crude material was purified via silica gel chromatography (0-80% ethyl acetate/DCM) to obtain 4-benzyloxy-2-[5-chloro-4-(2-hydroxy-1,1-dimethyl-ethyl)-2-methyl-phenyl]-1,6-naphthyridine-5-carboxamide (30 mg, 32%). ESI-MS m/z calc. 475.16, found 476.4 (M+1)⁺. The benzyl-protected intermediate was stirred with 10% Pd/C (wet, 25 mg) in ethanol (1 mL) under an atmosphere of hydrogen for 5 min. The mixture was filtered through a plug of celite and concentrated. Purification by silica gel chromatography (0-10% methanol/DCM) provided 2-[5-chloro-4-(2-hydroxy-1,1-dimethyl-ethyl)-2-methyl-phenyl]-4-oxo-1H-1,6-naphthyridine-5-carboxamide (356, 11 mg, 35%). ¹H NMR (400 MHz, DMSO-d₆) δ (ppm) 11.99 (s, 1H), 8.49 (d, J=5.8 Hz, 1H), 7.55-7.38 (m, 4H), 7.30 (s, 1H), 6.11 (s, 1H), 4.82 (d, J=5.3 Hz, 1H), 3.77 (d, J=5.0 Hz, 2H), 2.29 (s, 3H), 1.41 (s, 6H). ESI-MS m/z calc. 385.12, found 386.3 (M+1)⁺.

### Example 53

### 2-[5-chloro-2-methyl-4-(2,2,2-trifluoro-1,1-dimethyl-ethyl)phenyl]-4-oxo-1H-1,6-naphthyridine-5-carboxamide (357)

### Step 1: 2-[5-chloro-2-methyl-4-(2,2,2-trifluoro-1,1-dimethyl-ethyl)phenyl]-4-oxo-1H-1,6-naphthyridine-5-carboxamide (357)

A microwave vial charged with a mixture of 2-chloro-4-[(4-methoxyphenyl)methoxy]-1,6-naphthyridine-5-carbonitrile (385 mg, 1.18 mmol), 2-[5-chloro-2-methyl-4-(2,2,2-trifluoro-1,1-dimethyl-ethyl)phenyl]-4,4,5,5-tetramethyl-1,3,2-dioxaborolane (430 mg, 1.19 mmol), SPhos Pd G3 (110 mg, 0.141 mmol) and potassium acetate (630 mg, 2.97 mmol) in dioxane (8 mL) and water (800 μL) was flushed with nitrogen for 1 min, capped, and heated for 1 h at 50° C. The mixture was diluted with water and extracted with DCM (3×). The combined organic layers were dried over magnesium sulfate, filtered and concentrated. The crude material was dissolved in toluene (8 mL) and TFA (8 mL) and the mixture stirred at 60° C. for 6 hours. The mixture was concentrated and purified by silica gel chromatography (0-100% ethyl acetate/DCM) to provide 2-[5-chloro-2-methyl-4-(2,2,2-trifluoro-1,1-dimethyl-ethyl)phenyl]-4-oxo-1H-1,6-naphthyridine-5-carboxamide (357, 260 mg, 51%). ESI-MS m/z calc. 423.10, found 424.5 (M+1)⁺. ¹H NMR (400 MHz, DMSO-d₆) δ (ppm) 12.02 (s, 1H), 8.51 (d, J=5.8 Hz, 1H), 7.70 (s, 1H), 7.61 (s, 1H), 7.53 (s, 1H), 7.44 (d, J=5.8 Hz, 1H), 7.32 (s, 1H), 6.16 (s, 1H), 2.34 (s, 3H), 1.81 (s, 6H).

### Example 54

### 2-(6-tert-butyl-5-chloro-2-methyl-3-pyridyl)-4-oxo-1H-1,6-naphthyridine-5-carboxamide (358)

### Step 1: 2-(6-tert-butyl-5-chloro-2-methyl-3-pyridyl)-4-oxo-1H-1,6-naphthyridine-5-carboxamide (358)

A mixture of 4-benzyloxy-2-chloro-1,6-naphthyridine-5-carbonitrile (40 mg, 0.14 mmol), 2-tert-butyl-3-chloro-6-methyl-5-(4,4,5,5-tetramethyl-1,3,2-dioxaborolan-2-yl)pyridine (42 mg, 0.14 mmol) in aqueous potassium carbonate (350 μL of 1 M, 0.35 mmol) and dioxane (667 μL) was purged with nitrogen for 10 min. PdCl₂(dtbpf) (18 mg, 0.028 mmol) was added and the reaction was purged with nitrogen for an additional 5 min. The mixture was stirred at room temperature for 30 min, then partitioned between ethyl acetate and water. The organic layer was separated, washed with brine, dried over magnesium sulfate, filtered and concentrated. Purification using silica gel chromatography (0-20% ethyl acetate/hexanes) provided 4-benzyloxy-2-(6-tert-butyl-5-chloro-2-methyl-3-pyridyl)-1,6-naphthyridine-5-carbonitrile (50 mg, 83%) as a white solid. ESI-MS m/z calc. 442.16, found 443.5 (M+1)⁺. The intermediate was dissolved in toluene (400 μL) and TFA (600 μL, 7.79 mmol) heated at 60° C. for 15 h. The mixture was concentrated and purified using silica gel chromatography (0-80% ethyl acetate/DCM) to obtain 2-(6-tert-butyl-5-chloro-2-methyl-3-pyridyl)-4-oxo-1H-1,6-naphthyridine-5-carboxamide (358, 36 mg, 72%). ¹H NMR (500 MHz, DMSO-d₆) δ (ppm) 12.05 (s, 1H), 8.53 (d, J=5.8 Hz, 1H), 7.98 (s, 1H), 7.54 (s, 1H), 7.43 (d, J=5.7 Hz, 1H), 7.34 (s, 1H), 6.25 (s, 1H), 1.50 (s, 9H). ESI-MS m/z calc. 370.12, found 371.4 (M+1)⁺.

### Example 55

### 2-[5-(5-carbamoyl-4-oxo-1H-1,6-naphthyridin-2-yl)-3-chloro-6-methyl-2-pyridyl]-2-methyl-propanoic acid (359)

### Step 1: 2-[5-(5-carbamoyl-4-oxo-1H-1,6-naphthyridin-2-yl)-3-chloro-6-methyl-2-pyridyl]-2-methyl-propanoic acid (359)

To a solution of methyl 2-[5-(5-carbamoyl-4-oxo-1H-1,6-naphthyridin-2-yl)-3-chloro-6-methyl-2-pyridyl]-2-methyl-propanoate (56 mg, 0.13 mmol) in 1,4-dioxane (2.5 mL) was added potassium trimethylsilanolate (70 mg, 0.49 mmol). The vial was capped and placed in a pre-heated oil bath set at 100° C. for 7 hours, then stirred at room temperature for 16 hours. The reaction mixture was quenched with 5% aqueous citric acid (10 mL), diluted with ethyl acetate (10 mL) and stirred vigorously at room temperature for 45 minutes. The mixture was partitioned in additional 5% aqueous citric acid (15 mL) and ethyl acetate (15 mL), the layers were separated and the aqueous layer was extracted with ethyl acetate (2×25 mL), The combined organic layer was washed with water (25 mL), brine (25 mL), dried over sodium sulfate, filtered and concentrated under reduced pressure. Purification by reversed-phase chromatography (C₁₈) using 5 to 100% MeCN in water with (0.1% formic acid) followed by freeze-drying (acetonitrile/water mixture) afforded 2-[5-(5-carbamoyl-4-oxo-1H-1,6-naphthyridin-2-yl)-3-chloro-6-methyl-2-pyridyl]-2-methyl-propanoic acid (359, 31 mg, 56%) as a white solid. ESI-MS m/z calc. 400.0938, found 401.1 (M+1)⁺. ¹H NMR (400 MHz, DMSO-d₆) δ 12.62 (br. s, 1H), 12.10 (br. s, 1H), 8.51 (d, J=5.6 Hz, 1H), 8.02 (s, 1H), 7.52 (br. s, 1H), 7.42 (d, J=5.9 Hz, 1H), 7.31 (br. s, 1H), 6.26 (s, 1H), 2.51 (s, 3H), 1.57 (s, 6H).

### Example 56

### 2-[4-(2-adamantyl)-5-chloro-2-methyl-phenyl]-4-oxo-1H-1,6-naphthyridine-5-carboxamide (360)

### Step 1: 2-[4-(2-adamantyl)-5-chloro-2-methyl-phenyl]-4-benzyloxy-1,6-naphthyridine-5-carbonitrile

To a solution of 4-benzyloxy-2-[5-chloro-4-(2-hydroxy-2-adamantyl)-2-methyl-phenyl]-1,6-naphthyridine-5-carbonitrile (56 mg, 0.1 mmol) and triethylsilane (0.04 mL, 0.22 mmol) in DCM (0.6 mL) at −78° C. was added diethyloxonio(trifluoro)boranuide (0.025 mL, 0.20 mmol) dropwise. The reaction was gradually warmed to room temperature and stirred for 18 h. The reaction mixture was quenched by dropwise addition of saturated aqueous sodium bicarbonate solution, diluted with water and extracted with EtOAc. The combined organic layers were washed with brine, dried over magnesium sulfate, filtered and concentrated under reduced pressure. Purification by silica gel column chromatography using 0 to 50% EtOAc in hexanes provided 2-[4-(2-adamantyl)-5-chloro-2-methyl-phenyl]-4-benzyloxy-1,6-naphthyridine-5-carbonitrile (36 mg, 66%) ESI-MS m/z calc. 519.21, found 520.1 (M+1)⁺. ¹H NMR (400 MHz, CDCl₃) δ 8.84 (d, J=5.7 Hz, 1H), 8.11 (d, J=5.7 Hz, 1H), 7.63-7.56 (m, 2H), 7.53 (s, 1H), 7.49-7.41 (m, 3H), 7.39 (d, J=7.2 Hz, 1H), 7.10 (s, 1H), 5.52 (s, 2H), 3.33 (s, 1H), 2.35 (s, 2H), 2.28 (s, 3H), 2.03 (d, J=10.4 Hz, 5H), 1.96 (d, J=14.4 Hz, 3H), 1.81 (s, 2H), 1.70 (d, J=12.8 Hz, 2H).

### Step 2: 2-[4-(2-adamantyl)-5-chloro-2-methyl-phenyl]-4-oxo-1H-1,6-naphthyridine-5-carboxamide (360)

2-[4-(2-adamantyl)-5-chloro-2-methyl-phenyl]-4-oxo-1H-1,6-naphthyridine-5-carboxamide (360) was prepared from 2-[4-(2-adamantyl)-5-chloro-2-methyl-phenyl]-4-benzyloxy-1,6-naphthyridine-5-carbonitrile using a procedure analogous to that found in Example 4 (Method D, Step 2). ESI-MS m/z calc. 447.17, found 448.1 (M+1)⁺. ¹H NMR (400 MHz, CD₃OD) δ 8.53 (d, J=5.9 Hz, 1H), 7.66 (s, 1H), 7.52 (d, J=5.9 Hz, 1H), 7.47 (s, 1H), 6.31 (s, 1H), 3.37 (s, 1H), 2.36 (s, 4H), 2.11-1.99 (m, 7H), 1.95-1.84 (m, 4H), 1.75 (d, J=12.8 Hz, 2H).

### Example 57

### 2-[5-chloro-4-(2-fluoro-2-methyl-propyl)-2-methyl-phenyl]-4-oxo-1H-1,6-naphthyridine-5-carboxamide (361)

### Step 1: 4-benzyloxy-2-[5-chloro-4-(2-fluoro-2-methyl-propyl)-2-methyl-phenyl]-1,6-naphthyridine-5-carbonitrile

A microwave vial was charged with 4-benzyloxy-2-[5-chloro-4-(2-hydroxy-1,1-dimethyl-ethyl)-2-methyl-phenyl]-1,6-naphthyridine-5-carbonitrile (53.7 mg, 0.12 mmol) in dichloromethane (5 mL). The resulting solution was cooled to 0° C. and DAST (30 μL, 0.22 mmol) was added and the reaction mixture was stirred at 0° C. for 2 hours. The reaction mixture was quenched with a saturated solution of sodium bicarbonate, diluted with ethyl acetate. The layers were separated and the organic layer was washed with brine dried over anhydrous magnesium sulfate, filtered, and concentrated under reduced pressure. Purification by silica gel chromatography using 0 to 100% of EtOAc in hexanes gave 4-benzyloxy-2-[5-chloro-4-(2-fluoro-2-methyl-propyl)-2-methyl-phenyl]-1,6-naphthyridine-5-carbonitrile (20 mg, 32%). ESI-MS m/z calc. 459.15, found 460.0 (M+1)⁺.

### Step 2: 2-[5-chloro-4-(2-fluoro-2-methyl-propyl)-2-methyl-phenyl]-4-oxo-1H-1,6-naphthyridine-5-carboxamide (361)

2-[5-chloro-4-(2-fluoro-2-methyl-propyl)-2-methyl-phenyl]-4-oxo-1H-1,6-naphthyridine-5-carboxamide (361) was prepared from 4-benzyloxy-2-[5-chloro-4-(2-fluoro-2-methyl-propyl)-2-methyl-phenyl]-1,6-naphthyridine-5-carbonitrile using procedure analogous to that found in Example 4 (Method D, Step 2). ESI-MS m/z calc. 387.11, found 388.1 (M+1)⁺. ¹H NMR (400 MHz, CD₃OD) δ 8.54 (d, J=5.9 Hz, 1H), 7.55-7.48 (m, 2H), 7.41 (s, 1H), 6.31 (s, 1H), 3.18 (d, J=21.9 Hz, 2H), 2.33 (s, 3H), 1.38 (d, J=21.1 Hz, 6H). Decoupled: ¹⁹F NMR (376 MHz, CD₃OD) δ −138.53 (s, 1F). Coupled: ¹⁹F NMR (376 MHz, CD₃OD) δ −138.53 (hept, J=21.2 Hz, 1F).

### Example 58

### 2-(5-chloro-2-methyl-4-(-3-methylbicyclo[3.1.0]hexan-3-yl)phenyl)-4-oxo-1,4-dihydro-1,6-naphthyridine-5-carboxamide (362) & 2-(5-chloro-2-methyl-4-(-3-methylbicyclo[3.1.0]hexan-3-yl)phenyl)-4-oxo-1,4-dihydro-1,6-naphthyridine-5-carboxamide (363)

### Step 1: 4-benzyloxy-2-[5-chloro-2-methyl-4-(3-methyl-3-bicyclo[3.1.0]hexanyl)phenyl]-1,6-naphthyridin e-5-carboxamide

To a solution of 4-benzyloxy-2-[5-chloro-2-methyl-4-(3-methyl-3-bicyclo[3.1.0]hexanyl)phenyl]-1,6-naphthyridine-5-carbonitrile (26 mg, 0.05 mmol, mixture of cis/trans isomers) in DMSO (2 mL) in an oil bath set at 40° C. was added potassium carbonate (39 mg, 0.28 mmol) followed by the dropwise addition of aqueous hydrogen peroxide solution (480 mg, 0.4 mL of 35% w/w, 4.94 mmol). After 30 minutes, the reaction mixture was cooled to room temperature and partitioned between water (40 mL), brine (10 mL) and ethyl acetate (15 mL). The layers were separated and the aqueous layer was extracted again with ethyl acetate (2×15 mL). The combined organic layer was washed with water (20 mL), brine (20 mL), dried over sodium sulfate, filtered and concentrated under reduced pressure. The residue was purified by reverse-phase chromatography (C₁₈) using 5-100% MeCN in water (0.1% formic acid) to afford of 4-benzyloxy-2-[5-chloro-2-methyl-4-(3-methyl-3-bicyclo[3.1.0]hexanyl)phenyl]-1,6-naphthyridine-5-carboxamide (16 mg, 54%, mixture of cis/trans isomers) as a white solid. ESI-MS m/z calc. 497.187, found 498.2 (M+1)⁺.

### Step 2: SFC Separation: 4-benzyloxy-2-[5-chloro-2-methyl-4-(3-methyl-3-bicyclo[3.1.0]hexanyl)phenyl]-1,6-naphthyridine-5-carboxamide

4-benzyloxy-2-[5-chloro-2-methyl-4-(3-methyl-3-bicyclo[3.1.0]hexanyl)phenyl]-1,6-naphthyridine-5-carboxamide (31 mg) was subjected to SFC separation of the isomers using the following conditions: Phenomenex Lux Cellulose 5 column (250×21.2 mm), 5 μM column at 40° C., eluant: 40% MeOH, 60% CO₂, flow rate: 50 mL/min, concentration: 13.2 mg/mL in methanol (no modifier), injection volume: 200 μL, pressure: 100 bar, wavelength: 250 nm, 25 minute run time. Retention times of enantiomers were determined based on these conditions.

Peak 1 (isomer 1): 4-benzyloxy-2-[5-chloro-2-methyl-4-(3-methyl-3-bicyclo[3.1.0]hexanyl)phenyl]-1,6-naphthyridine-5-carboxamide (5 mg, 17%) as a white solid. ESI-MS m/z calc. 497.18, found 498.2 (M+1)⁺, Retention time: 16.67 minutes. ¹H NMR (400 MHz, CD₃OD) δ 8.66 (d, J=5.9 Hz, 1H), 7.90 (d, J=5.9 Hz, 1H), 7.59 (br d, J=7.3 Hz, 2H), 7.47-7.38 (m, 3H), 7.38-7.31 (m, 1H), 7.25 (d, J=7.8 Hz, 2H), 5.50 (s, 2H), 2.52 (br dd, J=14.2, 4.2 Hz, 2H), 2.20 (s, 3H), 1.79 (br d, J=13.2 Hz, 2H), 1.56-1.44 (m, 5H), 0.99-0.86 (m, 1H), 0.13 (app. q, J=3.6 Hz, 1H), two labile protons missing (CONH₂).

Peak 2 (isomer 2): 4-benzyloxy-2-[5-chloro-2-methyl-4-(3-methyl-3-bicyclo[3.1.0]hexanyl)phenyl]-1,6-naphthyridine-5-carboxamide (19 mg, 67%) as a white solid. ESI-MS m/z calc. 497.18, found 498.2 (M+1)⁺; Retention time: 18.65 minutes. ¹H NMR (400 MHz, CD₃OD) δ 8.66 (d, J=6.1 Hz, 1H), 7.91 (d, J=5.9 Hz, 1H), 7.59 (d, J=7.3 Hz, 2H), 7.47 (s, 1H), 7.45-7.39 (m, 2H), 7.39-7.31 (m, 2H), 7.28 (s, 1H), 5.51 (s, 2H), 2.73-2.60 (m, 2H), 2.22 (s, 3H), 1.95 (d, J=13.4 Hz, 2H), 1.52-1.43 (m, 2H), 1.40 (s, 3H), 0.70-0.58 (m, 1H), 0.49 (app. q, J=4.1 Hz, 1H), two labile protons missing (CONH₂).

### Step 3a: 2-(5-chloro-2-methyl-4-(3-methylbicyclo[3.1.0]hexan-3-yl)phenyl)-4-oxo-1,4-dihydro-1,6-naphthyridine-5-carboxamide (362)

A solution of 4-benzyloxy-2-[5-chloro-2-methyl-4-(3-methyl-3-bicyclo[3.1.0]hexanyl)phenyl]-1,6-naphthyridine-5-carboxamide (isomer 1, 16 mg, 0.0317 mmol) in degassed methanol (1.6 mL) was treated with palladium on carbon (6.4 mg, 5% w/w, 0.003 mmol) then placed under an atmosphere of hydrogen gas and stirred for 40 minutes. The reaction mixture was filtered and washed with methanol (about 5 mL). The solvent was removed under reduced pressure and the residue was purified by reverse-phase chromatography (C₁₈) using 5-100% MeCN in water (0.1% formic acid) to afford 2-[5-chloro-2-methyl-4-(3-methyl-3-bicyclo[3.1.0]hexanyl)phenyl]-4-oxo-1H-1,6-naphthyridine-5-carboxamide (362, 8 mg, 61%) as a white solid. ESI-MS m/z calc. 407.1401, found 408.1 (M+1)⁺. ¹H NMR (400 MHz, CD₃OD) δ 8.54 (d, J=5.9 Hz, 1H), 7.53 (br d, J=6.1 Hz, 1H), 7.47 (s, 1H), 7.43 (s, 1H), 6.29 (br s, 1H), 2.72-2.60 (m, 2H), 2.34 (s, 3H), 1.95 (d, J=13.7 Hz, 2H), 1.52-1.42 (m, 2H), 1.40 (s, 3H), 0.69-0.60 (m, 1H), 0.49 (app. q, J=3.9 Hz, 1H).

### Step 3b: 2-(5-chloro-2-methyl-4-(3-methylbicyclo[3.1.0]hexan-3-yl)phenyl)-4-oxo-1,4-dihydro-1,6-naphthyridine-5-carboxamide (363)

4-benzyloxy-2-[5-chloro-2-methyl-4-(3-methyl-3-bicyclo[3.1.0]hexanyl)phenyl]-1,6-naphthyridine-5-carboxamide (isomer 2, 5 mg, 0.009 mmol) in degassed methanol (0.5 mL) was treated with palladium on carbon (2 mg, 5% w/w, 939 nmmol) then placed under an atmosphere of hydrogen gas and stirred for 35 minutes. The reaction mixture was filtered and washed with methanol (about 2 mL). The solvent was removed under reduced pressure and the residue was purified by reverse-phase chromatography (C₁₈) using 5-100% MeCN in water (0.1% formic acid) to afford 2-[5-chloro-2-methyl-4-(3-methyl-3-bicyclo[3.1.0]hexanyl)phenyl]-4-oxo-1H-1,6-naphthyridine-5-carboxamide (363, 2.1 mg, 54%) as a white solid. ESI-MS m/z calc. 407.1401, found 408.1 (M+1)⁺. ¹H NMR (400 MHz, CD₃OD) δ 8.53 (br d, J=5.9 Hz, 1H), 7.53 (br d, J=5.9 Hz, 1H), 7.42 (s, 1H), 7.30 (s, 1H), 6.28 (s, 1H), 2.59-2.45 (m, 2H), 2.31 (s, 3H), 1.77 (br d, J=13.0 Hz, 2H), 1.57-1.41 (m, 5H), 1.00-0.85 (m, 1H), 0.17-0.05 (m, 1H).

### Example 59

### 2-[4-(5-carbamoyl-4-oxo-1H-1,6-naphthyridin-2-yl)-5-methyl-2-(trifluoromethyl)phenyl]-2-methyl-propanoic acid (364)

### Step 1: 2-[4-(5-cyano-4-oxo-1H-1,6-naphthyridin-2-yl)-5-methyl-2-(trifluoromethyl)phenyl]-2-methyl-propanoic acid

A solution of 4-benzyloxy-2-[4-(2-hydroxy-1,1-dimethyl-ethyl)-2-methyl-5-(trifluoromethyl)phenyl]-1,6-naphthyridine-5-carbonitrile (28 mg, 0.05 mmol) in acetone (2 mL) was cooled to 0° C. and Jones reagent (120 μL of 2 M, 0.24 mmol) was added dropwise. The mixture was stirred for 1 h at 0° C. and then 26 hours at room temperature. The mixture was then cooled down to 0° C. and quenched with isopropanol (5 mL). The mixture was stirred for 15 minutes at 0° C. The mixture was diluted with water (10 mL), extracted with ethyl acetate (2×10 mL). The organic phases were combined, dried over sodium sulfate, filtered, and concentrated under reduced pressure to afford 2-[4-(5-cyano-4-oxo-1H-1,6-naphthyridin-2-yl)-5-methyl-2-(trifluoromethyl)phenyl]-2-methyl-propanoic acid (28 mg, 84%). ESI-MS m/z calc. 415.11, found 416.1 (M+1)⁺. ¹H NMR (400 MHz, DMSO-d₆) δ 12.48 (br s, 1H), 8.73 (d, J=5.8 Hz, 1H), 7.79 (s, 1H), 7.73 (d, J=5.8 Hz, 1H), 7.69 (s, 1H), 6.34 (s, 1H), 2.43 (s, 3H), 1.61 (s, 6H), (one proton missing, labile proton). ¹⁹F NMR (377 MHz, DMSO-d₆) δ −74.11 (br s, 3F).

### Step 2: 2-[4-(5-carbamoyl-4-oxo-1H-1,6-naphthyridin-2-yl)-5-methyl-2-(trifluoromethyl)phenyl]-2-methyl-propanoic acid (364)

2-[4-(5-carbamoyl-4-oxo-1H-1,6-naphthyridin-2-yl)-5-methyl-2-(trifluoromethyl)phenyl]-2-methyl-propanoic acid (364, 9.4 mg, 45%) was prepared using procedure analogous to that found in Example 4, Method D (Step 2) as a tan solid. ESI-MS m/z calc. 433.12, found 434.2 (M+1)⁺. ¹H NMR (400 MHz, DMSO-d₆) δ 12.52 (s, 1H), 12.07 (s, 1H), 8.51 (d, J=5.6 Hz, 1H), 7.74 (s, 1H), 7.69 (s, 1H), 7.52 (br s, 1H), 7.43 (d, J=5.8 Hz, 1H), 7.31 (br s, 1H), 6.17 (d, J=1.5 Hz, 1H), 2.41 (s, 3H), 1.61 (s, 6H). ¹⁹F NMR (377 MHz, DMSO-d₆) δ −53.24 (s, 3F).

### Example 60

### rel-(R)-2-(5-chloro-2-methyl-4-(1,1,1-trifluoro-3-hydroxy-2-methylpropan-2-yl)phenyl)-4-oxo-1,4-dihydro-1,6-naphthyridine-5-carboxamide (365) & rel-(S)-2-(5-chloro-2-methyl-4-(1,1,1-trifluoro-3-hydroxy-2-methylpropan-2-yl)phenyl)-4-oxo-1,4-dihydro-1,6-naphthyridine-5-carboxamide (366)

### Step 1: rac-4-benzyloxy-2-[5-chloro-2-methyl-4-[2,2,2-trifluoro-1-(hydroxymethyl)-1-methyl-ethyl]phenyl]-1,6-naphthyridine-5-carbonitrile

A solution of TBAF in THF (0.35 mL of 1 M, 0.35 mmol) was added to a solution of 4-benzyloxy-2-[4-[1-[[tert-butyl(dimethyl)silyl]oxymethyl]-2,2,2-trifluoro-1-methyl-ethyl]-5-chloro-2-methyl-phenyl]-1,6-naphthyridine-5-carbonitrile (175 mg, 0.28 mmol) in THF (2.8 mL) at 0° C. The reaction was warmed to room temperature and stirred for 4 h. The reaction was diluted with EtOAc (20 mL), washed with water (20 mL), saturated aqueous ammonium chloride (10 mL), brine (10 mL), dried over anhydrous sodium sulfate, filtered and concentrated under reduced pressure to give crude rac-4-benzyloxy-2-[5-chloro-2-methyl-4-[2,2,2-trifluoro-1-(hydroxymethyl)-1-methyl-ethyl]phenyl]-1,6-naphthyridine-5-carbonitrile (151 mg). ESI-MS m/z calc. 511.12, found 512.2 (M+1)⁺.

### Step 2: SFC separation: 4-benzyloxy-2-[5-chloro-2-methyl-4-[2,2,2-trifluoro-1-(hydroxymethyl)-1-methyl-ethyl]phenyl]-1,6-naphthyridine-5-carbonitrile

rac-4-benzyloxy-2-[5-chloro-2-methyl-4-[2,2,2-trifluoro-1-(hydroxymethyl)-1-methyl-ethyl]phenyl]-1,6-naphthyridine-5-carbonitrile was subjected to SFC separation using Phenomenex Lux Column Amylose 1 (250×30 mm), 5 μM column at 40° C., eluent: 40% MeOH, 60% CO2, flow rate: 100 mL/min, concentration: 3 mg/mL in methanol (no modifier), injection volume: 2000 μL, pressure: 100 bar, wavelength: 220 nm. Retention times of enantiomers were determined by SFC using Amylose 1 column (30×250 mm, 5 um, 40° C., isocratic mobile phase of 40% MeOH, flow rate 3 mg/min, 9 min run time).

Peak 1 (enantiomer 1): rel-(R)-4-benzyloxy-2-[5-chloro-2-methyl-4-[2,2,2-trifluoro-1-(hydroxymethyl)-1-methyl-ethyl]phenyl]-1,6-naphthyridine-5-carbonitrile (59 mg, 41%) as a white solid. ESI-MS m/z calc. 511.1274, found 512.2 (M+1)⁺; Retention time: 4.43 minutes. ¹H NMR (400 MHz, CDCl₃) δ 8.87 (d, J=5.6 Hz, 1H), 8.08 (d, J=5.6 Hz, 1H), 7.59 (d, J=7.1 Hz, 2H), 7.53-7.50 (m, 2H), 7.48-7.37 (m, 3H), 7.08 (s, 1H), 5.54 (s, 2H), 4.69 (dd, J=12.2, 6.6 Hz, 1H), 4.32-4.24 (m, 1H), 2.26 (s, 3H), 1.81 (s, 3H), 1.64 (t, J=7.1 Hz, 1H). ¹⁹F NMR (377 MHz, CDCl₃) δ −70.56 (s, 3F). 99.9% ee.

Peak 2 (enantiomer 2): rel-(S)-4-benzyloxy-2-[5-chloro-2-methyl-4-[2,2,2-trifluoro-1-(hydroxymethyl)-1-methyl-ethyl]phenyl]-1,6-naphthyridine-5-carbonitrile (67 mg, 47%) as a white solid. ESI-MS m/z calc. 511.1274, found 512.2 (M+1)⁺; Retention time: 5.99 minutes. ¹H NMR (400 MHz, CDCl₃) δ 8.87 (d, J=5.6 Hz, 1H), 8.08 (d, J=5.6 Hz, 1H), 7.61-7.57 (m, 2H), 7.53-7.50 (m, 2H), 7.48-7.38 (m, 3H), 7.08 (s, 1H), 5.54 (s, 2H), 4.69 (dd, J=12.2, 6.4 Hz, 1H), 4.32-4.24 (m, 1H), 2.26 (s, 3H), 1.81 (s, 3H), 1.66-1.61 (m, 1H). ¹⁹F NMR (377 MHz, CDCl₃) δ −70.56 (s, 3F). 99.7% ee.

### Step 3a: rel-(R)-2-[5-chloro-2-methyl-4-[2,2,2-trifluoro-1-(hydroxymethyl)-1-methyl-ethyl]phenyl]-4-oxo-1H-1,6-naphthyridine-5-carboxamide (365)

rel-(R)-2-[5-chloro-2-methyl-4-[2,2,2-trifluoro-1-(hydroxymethyl)-1-methyl-ethyl]phenyl]-4-oxo-1H-1,6-naphthyridine-5-carboxamide (365) was prepared from 4-benzyloxy-2-[5-chloro-2-methyl-4-[2,2,2-trifluoro-1-(hydroxymethyl)-1-methyl-ethyl]phenyl]-1,6-naphthyridine-5-carbonitrile (Example 60, Step 2, Peak 1) using procedure analogous to that found in Example 4 (Method D, Step 2). ESI-MS m/z calc. 439.0911, found 440.1 (M+1)⁺. 99.9% ee. ¹H NMR (400 MHz, DMSO-d₆) δ 12.03 (br s, 1H), 8.50 (d, J=5.6 Hz, 1H), 7.67 (s, 1H), 7.56 (s, 1H), 7.51 (br s, 1H), 7.44 (br d, J=5.4 Hz, 1H), 7.31 (br s, 1H), 6.15 (s, 1H), 5.30 (t, J=5.4 Hz, 1H), 4.53 (dd, J=11.0, 5.6 Hz, 1H), 3.96 (dd, J=10.8, 4.9 Hz, 1H), 2.32 (s, 3H), 1.79 (s, 3H). ¹⁹F NMR (377 MHz, DMSO-d₆) δ −69.55 (s, 3F).

### Step 3b: rel-(S)-2-[5-chloro-2-methyl-4-[2,2,2-trifluoro-1-(hydroxymethyl)-1-methyl-ethyl]phenyl]-4-oxo-1H-1,6-naphthyridine-5-carboxamide (366)

rel-(S)-2-[5-chloro-2-methyl-4-[2,2,2-trifluoro-1-(hydroxymethyl)-1-methyl-ethyl]phenyl]-4-oxo-1H-1,6-naphthyridine-5-carboxamide (366) was prepared from 4-benzyloxy-2-[5-chloro-2-methyl-4-[2,2,2-trifluoro-1-(hydroxymethyl)-1-methyl-ethyl]phenyl]-1,6-naphthyridine-5-carbonitrile (Example 60, Step 2, Peak 2), Example using procedure analogous to that found in Example 4 (Method D, Step 2). ESI-MS m/z calc. 439.0911, found 440.1 (M+1)⁺. 99.9% ee. ¹H NMR (400 MHz, DMSO-d₆) δ 12.04 (br s, 1H), 8.50 (d, J=5.9 Hz, 1H), 7.67 (s, 1H), 7.56 (s, 1H), 7.52 (br s, 1H), 7.44 (br d, J=5.9 Hz, 1H), 7.31 (br s, 1H), 6.16 (s, 1H), 5.31 (t, J=5.5 Hz, 1H), 4.53 (dd, J=11.2, 5.6 Hz, 1H), 3.97 (dd, J=11.0, 5.1 Hz, 1H), 2.33 (s, 3H), 1.80 (s, 3H). ¹⁹F NMR (377 MHz, DMSO-d₆) δ −69.55 (s, 3F).

### Example 61

### rac-2-[4-(5-carbamoyl-4-oxo-1H-1,6-naphthyridin-2-yl)-2-chloro-5-methyl-phenyl]-3,3,3-trifluoro-2-methyl-propanoic acid (367)

### Step 1: rac-2-[4-(5-carbamoyl-4-oxo-1H-1,6-naphthyridin-2-yl)-2-chloro-5-methyl-phenyl]-3,3,3-trifluoro-2-methyl-propanoic acid (367)

rac-2-[4-(5-carbamoyl-4-oxo-1H-1,6-naphthyridin-2-yl)-2-chloro-5-methyl-phenyl]-3,3,3-trifluoro-2-methyl-propanoic acid (367, 11.2 mg, 19%) was prepared from 4-benzyloxy-2-[5-chloro-2-methyl-4-[2,2,2-trifluoro-1-(hydroxymethyl)-1-methyl-ethyl]phenyl]-1,6-naphthyridine-5-carbonitrile using procedure analogous to that found in Example 59 (Step 1 and Step 2) as a white solid. ESI-MS m/z calc. 453.07, found 454.1 (M+1)⁺. ¹H NMR (400 MHz, DMSO-d₆) δ 13.77 (br s, 1H), 12.05 (s, 1H), 8.50 (d, J=5.9 Hz, 1H), 7.68 (br s, 1H), 7.61 (s, 1H), 7.52 (br s, 1H), 7.43 (d, J=5.9 Hz, 1H), 7.31 (br s, 1H), 6.18 (s, 1H), 2.35 (s, 3H), 1.89 (br s, 3H). ¹⁹F NMR (377 MHz, DMSO-d₆) δ −68.89 (br s, 3F).

### Example 62

### 2-(4-tert-butyl-5-chloro-2-methyl-phenyl)-5-methoxy-1H-1,6-naphthyridin-4-one (368)

### Step 1: 2-(4-tert-butyl-5-chloro-2-methyl-phenyl)-5-methoxy-1H-1,6-naphthyridin-4-one (368)

2-(4-tert-butyl-5-chloro-2-methyl-phenyl)-5-methoxy-1H-1,6-naphthyridin-4-one (368) was prepared from 4-(benzyloxy)-2-(4-(tert-butyl)-5-chloro-2-methylphenyl)-5-chloro-1,6-naphthyridine using procedure analogous to Example 11 (Step1 and Step 2). ESI-MS m/z calc. 356.13, found 357.40 (M+1)⁺. ¹H NMR (400 MHz, CD₃OD) δ 7.91 (d, J=7.7 Hz, 1H), 7.50 (d, J=4.5 Hz, 2H), 7.07 (s, 1H), 6.78 (d, J=7.6 Hz, 1H), 3.68 (s, 3H), 2.35 (s, 3H), 1.52 (s, 9H).

### Example 63

### [2-(4-tert-butyl-5-chloro-2-methyl-phenyl)-4-oxo-1H-1,6-naphthyridin-5-yl]urea (369)

### Step 1: [4-benzyloxy-2-(4-tert-butyl-5-chloro-2-methyl-phenyl)-1,6-naphthyridin-5-yl]urea

A microwave vial was charged with urea (30 mg, 0.5 mmol) and anhydrous dioxane (4 mL) and the suspension was sparged with nitrogen-gas for 5 minutes. To this suspension was then sequentially added cesium carbonate (200 mg, 0.61 mmol), Xantphos (20 mg, 0.035 mmol), and Pd₂(dba)₃ (5 mg, 0.005 mmol). The resultant mixture was sparged again with nitrogen-gas for 5 minutes and was heated at 30° C. for 30 min (pre-complex formation). To this reaction mixture at 30° C. was added 4-benzyloxy-2-(4-tert-butyl-5-chloro-2-methyl-phenyl)-5-chloro-1,6-naphthyridine (150 mg, 0.33 mmol) and the reaction mixture was heated at 95° C. overnight. Upon completion, the reaction mixture was cooled to room temperature, filtered through a pad of Celite and the pad was thoroughly washed with ethyl acetate. The filtrate was concentrated under reduced pressure and purified via silica gel column chromatography using 0 to 70% EtOAc in hexanes to obtain [4-benzyloxy-2-(4-tert-butyl-5-chloro-2-methyl-phenyl)-1,6-naphthyridin-5-yl]urea (84 mg, 53%) ESI-MS m/z calc. 474.18, found 475.5 (M+1)⁺.

### Step 2: [2-(4-tert-butyl-5-chloro-2-methyl-phenyl)-4-oxo-1H-1,6-naphthyridin-5-yl]urea (369)

[4-benzyloxy-2-(4-tert-butyl-5-chloro-2-methyl-phenyl)-1,6-naphthyridin-5-yl]urea (80 mg, 0.17 mmol) was taken up in toluene (700 μL) and TFA (450 μL) was added (turns yellow). The resulting reaction mixture was heated at 70° C. for 1 hour. The solvent was evaporated, and the crude material was taken up in DCM (10 mL) and quenched with saturated aqueous. sodium bicarbonate solution. The layers were separated, and the aqueous layer was extracted with DCM (2×). The combined organic layer was dried over magnesium sulfate, filtered and concentrated. Purification via silica gel column chromatography using 0 to 5% MeOH in DCM gave [2-(4-tert-butyl-5-chloro-2-methyl-phenyl)-4-oxo-1H-1,6-naphthyridin-5-yl]urea (369, 25 mg, 39%). ESI-MS m/z calc. 384.13, found 385.5 (M+1)⁺. ¹H NMR (400 MHz, DMSO-d₆) δ 12.24 (s, 1H), 12.16 (s, 1H), 8.99 (s, 1H), 8.16 (d, J=6.0 Hz, 1H), 7.51 (s, 1H), 7.48 (s, 1H), 7.21 (s, 1H), 6.97 (d, J=6.0 Hz, 1H), 6.27 (s, 1H), 2.30 (s, 3H), 1.48 (s, 9H).

### Example 64

### 2-(4-tert-butyl-5-chloro-2-methyl-phenyl)-5-hydroxy-1H-1,6-naphthyridin-4-one (370)

### Step 1: 2-(4-tert-butyl-5-chloro-2-methyl-phenyl)-5-hydroxy-1H-1,6-naphthyridin-4-one (370)

2-(4-tert-butyl-5-chloro-2-methyl-phenyl)-5-hydroxy-1H-1,6-naphthyridin-4-one (370) was prepared using procedure analogous to that found in Example 10 (Step 1 and 2), from 4-benzyloxy-2-chloro-6-oxido-1,6-naphthyridin-6-ium. ESI-MS m/z calc. 342.11, found 343.32 (M+1)⁺. ¹H NMR (400 MHz, DMSO-d₆) δ 7.68 (d, J=7.4 Hz, 1H), 7.51 (s, 1H), 7.45 (s, 1H), 7.10 (s, 1H), 6.77 (d, J=7.3 Hz, 1H), 2.35 (s, 3H), 1.49 (s, 9H).

### Example 65

### 1-[2-(4-tert-butyl-5-chloro-2-methyl-phenyl)-4-oxo-1H-1,6-naphthyridin-5-yl]-3-methyl-urea (371)

### Step 1: 1-[2-(4-tert-butyl-5-chloro-2-methyl-phenyl)-4-oxo-1H-1,6-naphthyridin-5-yl]-3-methyl-urea (371)

1-[2-(4-tert-butyl-5-chloro-2-methyl-phenyl)-4-oxo-1H-1,6-naphthyridin-5-yl]-3-methyl-urea (371) can be prepared using methylurea, using a procedure analogous to that found in Example 63 (Step1 and Step 2). ESI-MS m/z calc. 398.15, found 399.4 (M+1)⁺. ¹H NMR (400 MHz, DMSO-d₆) δ 12.24 (d, J=2.6 Hz, 2H), 9.46 (q, J=4.5 Hz, 1H), 8.15 (d, J=6.0 Hz, 1H), 7.51 (s, 1H), 7.48 (s, 1H), 6.98 (d, J=6.1 Hz, 1H), 6.27 (s, 1H), 2.82 (d, J=4.6 Hz, 3H), 2.30 (s, 3H), 1.48 (s, 9H).

### Example 66

### 2-(4-tert-butyl-5-chloro-2-methyl-phenyl)-N-methyl-4-oxo-1H-1,6-naphthyridine-5-carboxamide (372)

### Step 1: 2-(4-tert-butyl-5-chloro-2-methyl-phenyl)-N-methyl-4-oxo-1H-1,6-naphthyridine-5-carboxamide (372)

2-(4-tert-butyl-5-chloro-2-methyl-phenyl)-N-methyl-4-oxo-1H-1,6-naphthyridine-5-carboxamide (372) was prepared using a procedure analogous to that found in Example 25 (Step 2), using 2-(4-tert-butyl-5-chloro-2-methyl-phenyl)-4-oxo-1H-1,6-naphthyridine-5-carboxylic acid. ESI-MS m/z calc. 383.14, found 384.42 (M+1)⁺. ¹H NMR (400 MHz, CD₃OD) δ 8.54 (d, J=5.9 Hz, 1H), 7.53 (d, J=5.9 Hz, 1H), 7.50 (s, 1H), 7.46 (s, 1H), 6.29 (s, 1H), 2.99 (s, 3H), 2.34 (s, 3H), 1.52 (s, 9H). (N−H proton exchangeable in methanol).

### Example 67

### 2-(4-tert-butyl-5-chloro-2-methyl-phenyl)-5-(dimethylamino)-1H-1,6-naphthyridin-4-one (373)

### Step 1: 2-(4-tert-butyl-5-chloro-2-methyl-phenyl)-5-(dimethylamino)-1H-1,6-naphthyridin-4-one (373)

N-methylmethanamine (hydrochloride salt) (11 μL, 0.13 mmol) was dissolved in THF (1 mL) and to it was added sodium hydride (3 mg of 60% w/w, 0.075 mmol) at 0° C. and stirred for 30 minutes. To the stirred reaction mixture was added 4-benzyloxy-2-(4-tert-butyl-5-chloro-2-methyl-phenyl)-5-chloro-1,6-naphthyridine (19.1 mg, 0.04 mmol) at 0° C. The reaction mixture was gradually warmed to room temperature over 30 minutes and was quenched with methanol. Purification via reverse phase silica gel column chromatography using 1 to 100% ACN in water containing 5 mM hydrochloric acid gave 4-benzyloxy-2-(4-tert-butyl-5-chloro-2-methyl-phenyl)-N,N-dimethyl-1,6-naphthyridin-5-amine as a clear colorless solid. ESI-MS m/z calc. 459.20, found 460.58 (M+1)⁺.

2-(4-tert-butyl-5-chloro-2-methyl-phenyl)-5-(dimethylamino)-1H-1,6-naphthyridin-4-one (374) was prepared using procedure analogous to that found in Example 4 (Method C, Step 2), from 4-benzyloxy-2-(4-tert-butyl-5-chloro-2-methyl-phenyl)-N,N-dimethyl-1,6-naphthyridin-5-amine. ESI-MS m/z calc. 369.1608, found 370.391 (M+1)⁺. ¹H NMR (400 MHz, CD₃OD) δ 8.00 (d, J=5.7 Hz, 1H), 7.47 (s, 1H), 7.42 (s, 1H), 6.69 (d, J=5.8 Hz, 1H), 6.19 (s, 1H), 3.07 (s, 6H), 2.33 (s, 3H), 1.52 (s, 9H).

### Example 68

### 2-(4-tert-butyl-5-chloro-2-methyl-phenyl)-4-oxo-1H-1,6-naphthyridine-5-carboxamidine (374)

### Step 1: 2-(4-tert-butyl-5-chloro-2-methyl-phenyl)-4-oxo-1H-1,6-naphthyridine-5-carboxamidine (374)

A microwave vial charged with 4-benzyloxy-2-(4-tert-butyl-5-chloro-2-methyl-phenyl)-1,6-naphthyridine-5-carbonitrile (50 mg, 0.11 mmol) and toluene (1.5 mL) was degassed under vacuum and backfilled with nitrogen. To it was added [amino(chloro)alumanyl]methane (1 mL of 0.67 M, 0.67 mmol) under an inert atmosphere. The reaction mixture was heated at 80° C. overnight. The reaction mixture was quenched with sat. aqueous ammonium chloride solution. The product crashed out, which was filtered, dissolved in DMSO and purified via reverse phase column chromatography using 0 to 99% ACN in water (HCl modifier) to obtain 2-(4-tert-butyl-5-chloro-2-methyl-phenyl)-4-oxo-1H-1,6-naphthyridine-5-carboxamidine (hydrochloride salt) (374, 10.5 mg, 23%). ESI-MS m/z calc. 368.14, found 370.3 (M+2)⁺. H NMR (400 MHz, DMSO-d₆) δ 12.51 (s, 1H), 9.22 (s, 2H), 9.09 (s, 2H), 8.70 (d, J=5.8 Hz, 1H), 7.75 (d, J=5.8 Hz, 1H), 7.51 (s, 2H), 6.29 (s, 1H), 2.31 (s, 3H), 1.49 (s, 9H).

### Example 69

### 2-(4-tert-butyl-5-chloro-2-methyl-phenyl)-8-methyl-4-oxo-1H-1,6-naphthyridine-3-carboxylic acid (375), 2-(4-tert-butyl-5-chloro-2-methyl-phenyl)-8-methyl-1H-1,6-naphthyridin-4-one (376), & 2-(4-tert-butyl-5-chloro-2-methyl-phenyl)-8-methyl-4-oxo-1H-1,6-naphthyridine-5-carboxamide (377)

### Step 1: methyl 4-amino-5-bromo-pyridine-3-carboxylate

A mixture of methyl 4-aminopyridine-3-carboxylate (17.52 g, 115.15 mmol) in AcOH (150 mL) and water (150 mL) was heated to 75° C. until clear. The temperature was reduced to 50° C. and bromine (62 g, 20 mL, 388 mmol) was added dropwise. The reaction was heated at 50° C. overnight. The reaction mixture was cooled to room temperature and was placed in an ice-bath. The red solid was collected by filtration, washed with water and dried to obtain methyl 4-amino-5-bromo-pyridine-3-carboxylate (Bromide Ion (1)) (35.12 g, 96%) ¹H-NMR (400 MHz, DMSO-d₆) δ 8.80 (s, 1H), 8.73 (s, 1H), 9.00-8.30 (m, 2H).

### Step 2: methyl 4-amino-5-methyl-pyridine-3-carboxylate

To a mixture of methyl 4-amino-5-bromo-pyridine-3-carboxylate (200 mg, 0.82 mmol) and 2,4,6-trimethyl-1,3,5,2,4,6-trioxatriborinane (435 mg, 485 μL, 3.5 mmol) in dioxane (2.2 mL) and water (0.22 mL) was added sodium carbonate (412 mg, 3.9 mmol). The reaction mixture was purged with nitrogen gas for 10 min and Pd(dppf)Cl₂·DCM (71 mg, 0.09 mmol) was added. The reaction mixture was stirred at 100° C. for 4 hours. It was then cooled to room temperature, filtered through Celite and washed with EtOAc (25 mL). The filtrate was concentrated under reduced pressure to obtain methyl 4-amino-5-methyl-pyridine-3-carboxylate (149 mg, 83%) as brown solid. ESI-MS m/z calc. 166.07, found 167.1 (M+1)⁺.

### Step 3: 4-amino-5-methyl-pyridine-3-carboxylic acid

To a solution of methyl 4-amino-5-methyl-pyridine-3-carboxylate (2 g, 10.40 mmol) in THF (20 mL), MeOH (10 mL) and water (10 mL) was added lithium hydroxide monohydrate (654 mg, 15.59 mmol). The resulting mixture was stirred at room temperature overnight. The volatiles were removed in vacuo and the pH of the residue was adjusted to 4-5 using 2N HCl. The resulting solid was collected by filtration, washed with water (20 mL) and dried over high vacuum to obtain 4-amino-5-methyl-pyridine-3-carboxylic acid (1.13 g, 68%) as brown solid. ¹H-NMR (400 MHz, DMSO-d₆) δ 8.48 (s, 1H), 7.94 (s, 1H), 7.53-7.45 (m, 2H), 2.04 (s, 3H). 1H not observed.

### Step 4: 2-(4-tert-butyl-5-chloro-2-methyl-phenyl)-8-methyl-pyrido[4,3-d][1,3]oxazin-4-one

A mixture of 4-amino-5-methyl-pyridine-3-carboxylic acid (1.1 g, 6.87 mmol) and pyridine (30 mL) was heated at 60° C. until homogeneous. 4-tert-butyl-5-chloro-2-methyl-benzoyl chloride (1.95 g, 7.56 mmol) was added and stirring was continued for 16 h. On cooling to room temperature, water (25 mL) was added and the mixture was cooled to 0° C. (ice-water). The resulting brown precipitate was collected by filtration, rinsed with water (5 mL) and dried to give 2-(4-tert-butyl-5-chloro-2-methyl-phenyl)-8-methyl-pyrido[4,3-d][1,3]oxazin-4-one (1.1 g, 46%). ESI-MS m/z calc. 342.1135, found 343.14 (M+1)⁺. ¹H-NMR (400 MHz, DMSO-d₆) δ 9.11 (s, 1H), 8.86 (s, 1H), 7.91 (s, 1H), 7.48 (s, 1H), 2.67 (s, 3H), 1.45 (s, 9H). 3H probably overlapped with the water peak.

### Step 5: ethyl 3-[4-[(4-tert-butyl-5-chloro-2-methyl-benzoyl)amino]-5-methyl-3-pyridyl]-3-oxo-propanoate

A mixture of THF (25 mL) and diisopropylamine (722 mg, 1 mL, 7.13 mmol) was cooled to −78° C. and n-BuLi in hexanes (2.8 mL of 2.5 M, 7 mmol) was added. After stirring at −78° C. for 20 min, ethyl acetate (631.40 mg, 0.7 mL, 7.16 mmol) was added and stirring was continued for 5 minutes. 2-(4-tert-butyl-5-chloro-2-methyl-phenyl)-8-methyl-pyrido[4,3-d][1,3]oxazin-4-one (500 mg, 1.43 mmol) was added and after 1 h, the reaction mixture was gradually warmed to room tmeperatue. 1N NaOH (10 mL) was added and the reaction mixture was stirred for 16 h. The mixture was poured into brine (10 mL) and extracted with EtOAc (2×20 mL). The combined organic layer was, dried over Na2SO4, filtered and concentrated to give ethyl 3-[4-[(4-tert-butyl-5-chloro-2-methyl-benzoyl)amino]-5-methyl-3-pyridyl]-3-oxo-propanoate (570 mg, 83%). ESI-MS m/z calc. 430.16, found 431.22 (M+1)⁺. ¹H-NMR (400 MHz, DMSO-d₆) δ 11.18 (s, 1H), 8.43 (s, 1H), 8.34 (d, J=13.7 Hz, 1H), 7.59 (s, 1H), 7.34 (s, 1H), 4.90 (s, 1H), 3.90 (q, J=7.0 Hz, 2H), 2.40 (s, 3H), 2.15 (s, 3H), 1.40 (d, J=15.1 Hz, 9H), 1.14-1.06 (m, 3H).

### Step 6: ethyl 2-(4-tert-butyl-5-chloro-2-methyl-phenyl)-8-methyl-4-oxo-1H-1,6-naphthyridine-3-carboxylate

A suspension of ethyl 3-[4-[(4-tert-butyl-5-chloro-2-methyl-benzoyl)amino]-5-methyl-3-pyridyl]-3-oxo-propanoate (530 mg, 1.1 mmol) in AcOH (20 mL) was heated at 90° C. for 4 hours. On cooling, the mixture was concentrated under vacuum to give ethyl 2-(4-tert-butyl-5-chloro-2-methyl-phenyl)-8-methyl-4-oxo-1H-1,6-naphthyridine-3-carboxylate (450 mg, 91%). ESI-MS m/z calc. 412.15, found 413.22 (M+1)⁺. ¹H-NMR (400 MHz, CDCl₃) δ 9.45 (s, 1H), 8.60 (d, J=10.1 Hz, 1H), 7.30 (s, 1H), 7.26 (s, 1H), 4.07 (q, J=7.2 Hz, 2H), 2.57-2.50 (m, 3H), 2.22 (d, J=4.1 Hz, 3H), 1.47 (s, 9H), 0.88 (t, J=7.1 Hz, 3H).

### Step 7: 2-(4-tert-butyl-5-chloro-2-methyl-phenyl)-8-methyl-4-oxo-1H-1,6-naphthyridine-3-carboxylic acid (375)

To ethyl 2-(4-tert-butyl-5-chloro-2-methyl-phenyl)-8-methyl-4-oxo-1H-1,6-naphthyridine-3-carboxylate (350 mg, 0.78 mmol) in water (9 mL) was added hydrochloric acid (37% in water) (109.38 g, 3 mL of 37% w/w, 1.11 mol) and the resultant solution was heated to 90° C. overnight. Additional hydrochloric acid (218.76 g, 6 mL of 37% w/w, 2.22 mol) was added and the mixture stirred at 105° C. for another 2 hours. On cooling, the resulting precipitate was collected by filtration, washed with water (2×5 mL) and dried to give 2-(4-tert-butyl-5-chloro-2-methyl-phenyl)-8-methyl-4-oxo-1H-1,6-naphthyridine-3-carboxylic acid (375, 247 mg, 78%). ESI-MS m/z calc. 384.1241, found 385.16 (M+1)⁺. ¹H-NMR (400 MHz, CD₃OD) δ 9.47 (s, 1H), 8.69 (s, 1H), 7.46 (s, 1H), 7.39 (s, 1H), 2.61 (s, 3H), 2.26 (s, 3H), 1.51 (s, 9H). Exchangeable H's (2H) not observed.

### Step 8: 2-(4-tert-butyl-5-chloro-2-methyl-phenyl)-8-methyl-1H-1,6-naphthyridin-4-one (376)

Dowtherm A (2 mL) was heated to 250° C. and to this was added 2-(4-tert-butyl-5-chloro-2-methyl-phenyl)-8-methyl-4-oxo-1H-1,6-naphthyridine-3-carboxylic acid (140 mg, 0.3437 mmol) portion wise (immediate gas evolution) and stirred for 10 minutes. After cooling to room temperature, the mixture was diluted with heptane (10 mL) and the resulting precipitate was collected by filtration, washed with heptane (2 mL) and dried to give 2-(4-tert-butyl-5-chloro-2-methyl-phenyl)-8-methyl-1H-1,6-naphthyridin-4-one (376, 100 mg, 82%). ESI-MS m/z calc. 340.1342, found 341.15 (M+1)⁺. ¹H-NMR (400 MHz, CD₃OD) δ 9.25 (s, 1H), 8.48 (s, 1H), 7.47 (d, J=5.5 Hz, 2H), 6.32 (s, 1H), 2.51 (s, 3H), 2.32 (s, 3H), 1.51 (s, 9H). 1H not observed.

### Step 9: 2-(4-tert-butyl-5-chloro-2-methyl-phenyl)-8-methyl-4-oxo-1H-1,6-naphthyridine-5-carboxamide (377)

A solution of 2-(4-tert-butyl-5-chloro-2-methyl-phenyl)-8-methyl-1H-1,6-naphthyridin-4-one (60 mg, 0.17 mmol) in formamide (3 mL) was heated to 80° C. Potassium persulfate (98 mg, 0.36 mmol) was added portion wise over 30 mins. The reaction mixture was then stirred for 30 mins at 80° C. On cooling, the mixture was quenched with a saturated aqueous solution of sodium hydrogencarbonate (10 mL) and the aqueous layer was extracted with EtOAc (20 mL×3). The combined organic layers were washed with brine, dried over anhydrous sodium sulfate, filtered and concentrated. The crude material was purified by reverse phase chromatography (C₁₈) using 10-70% MeCN in H₂O each containing 0.1% ammonium hydroxide to give 2-(4-tert-butyl-5-chloro-2-methyl-phenyl)-8-methyl-4-oxo-1H-1,6-naphthyridine-5-carboxamide (377, 15 mg, 23%). ESI-MS m/z calc. 383.14, found 384.19 (M+1)⁺. ¹H NMR (400 MHz, CD₃OD) δ 8.42 (s, 1H), 7.46 (s, 1H), 7.45 (s, 1H), 6.27 (br s, 1H), 2.51 (s, 3H), 2.33 (s, 3H), 1.51 (s, 9H).

### Example 70

### 2-(4-tert-butyl-5-chloro-2-methyl-phenyl)-5-(1-methylimidazol-2-yl)-1H-1,6-naphthyridin-4-one (378)

### Step 1: 2-(4-tert-butyl-5-chloro-2-methyl-phenyl)-5-(1-methylimidazol-2-yl)-1H-1,6-naphthyridin-4-one (378)

A solution of Pd(PPh₃)₄(13.1 mg, 0.01 mmol) and 4-benzyloxy-2-(4-tert-butyl-5-chloro-2-methyl-phenyl)-5-chloro-1,6-naphthyridine (50 mg, 0.11 mmol) in toluene (0.5 mL) was sparged with nitrogen for 3 minutes. A solution of tributyl-(1-methylimidazol-2-yl)stannane (49 mg, 0.13 mmol) in toluene (0.8 mL) was added to the reaction mixture and the reaction was stirred at 100° C. for 4 hours. The reaction mixture was heated at 70° C. for 2 hours, concentrated under reduced pressure, diluted with DMSO (2 mL), filtered, and purified by reverse phase preparative column chromatography (C₁₈) using 1-99% MeCN in water (HCl modifier) to give 2-(4-tert-butyl-5-chloro-2-methyl-phenyl)-5-(1-methylimidazol-2-yl)-CH-1,6-naphthyridin-4-one (Hydrochloride salt) (378, 2.5 mg, 43). ESI-MS m/z calc. 406.15, found 407.3 (M+1)⁺. ¹H-NMR (400 MHz, DMSO-d₆) δ 12.07 (s, 1H), 8.64 (d, J=5.7 Hz, 1H), 7.58 (d, J=5.7 Hz, 1H), 7.52 (s, 1H), 7.48 (s, 1H), 7.17 (s, 1H), 6.89 (s, 1H), 6.09 (s, 1H), 3.37 (s, 3H), 2.32 (s, 3H), 1.48 (s, 9H).

The following compounds were synthesized using the route shown in Example 70, using the appropriate heterocyclic stannanes. Stille coupling followed by deprotection provided the following compounds.

TABLE 6

| | | LC/MS | |
|---|---|---|---|
| Cmpd. | | (m/z calc.), | |
| No. | Compound Name | Found M + 1]⁺ | NMR (shifts in ppm) |
| 379 | 2-(6-tert-butyl-5- | 407.15 | ¹H NMR (400 MHz, CDCl₃) δ 13.61 (s, 1H), |
| | chloro-2-methyl-3- | 408 | 8.39 (d, J = 5.7 Hz, 1H), 8.34 (d, J = 6.0 Hz, |
| | pyridyl)-5-(1- | | 1H), 7.89 (s, 1H), 7.37 (d, J = 2.0 Hz, 1H), |
| | methylimidazol-2- | | 7.19 (d, J = 2.0 Hz, 1H), 6.36 (s, 1H), 3.74 |
| | y1)-1H-1,6- | | (s, 3H), 2.59 (s, 3H), 1.51 (s, 9H). |
| | naphthyridin-4-one | | |
| 380 | 2-(4-tert-buty1-5- | 392.13 | ¹H NMR (400 MHz, DMSO-d₆) δ 12.05 (s, |
| | chloro-2-methyl- | 393.3 | 1H), 8.08 (d, J = 0.9 Hz, 1H), 7.85-7.78 |
| | phenyl)-5-oxazol-2- | | (m, 1H), 7.78-7.71 (m, 1H), 7.49 (d, J = |
| | y1-1H-quinolin-4- | | 4.3 Hz, 2H), 7.38 (dd, J = 7.1, 1.2 Hz, 1H), |
| | one | | 7.27 (d, J = 0.9 Hz, 1H), 6.04 (s, 1H), 2.31 |
| | | | (s, 3H), 1.49 (s, 9H). |
| 381 | 2-(4-tert-butyl-5- | 440.12 | — |
| | chloro-2-methyl- | 441.6 | |
| | phenyl)-5-(5-chloro- | | |
| | 1-methyl-imidazol- | | |
| | 2-yl)-1H-1,6- | | |
| | naphthyridin-4-one | | |
| 382 | 2-(4-tert-butyl-5- | 403.15 | ¹H NMR (400 MHz, CD₃OD) δ 8.93-8.87 |
| | chloro-2-methyl- | 404.3 | (m, 1H), 8.84 (d, J = 5.9 Hz, 1H), 8.70- |
| | phenyl)-5-(2- | | 8.61 (m, 1H), 8.37 (d, J = 8.1 Hz, 1H), 8.16- |
| | pyridyl)-1H-1,6- | | 8.08 (m, 1H), 7.81 (d, J = 5.9 Hz, 1H), |
| | naphthyridin-4-one | | 7.53 (s, 1H), 7.51 (s, 1H), 6.42 (s, 1H), 2.37 |
| | | | (s, 3H), 1.53 (s, 9H). |
| 383 | 2-(4-tert-butyl-5- | 417.16 | ¹H NMR (400 MHz, CD₃OD) δ 8.84 (d, J = |
| | chloro-2-methyl- | 418.3 | 5.9 Hz, 1H), 8.75-8.69 (m, 1H), 8.57 (d, J = |
| | phenyl)-5-(3-methyl- | | 8.0 Hz, 1H), 8.09-8.01 (m, 1H), 7.81 (d, |
| | 2-pyridyl)-1H-1,6- | | J = 5.9 Hz, 1H), 7.51 (d, J = 11.3 Hz, 2H), |
| | naphthyridin-4-one | | 6.30 (s, 1H), 2.36 (s, 3H), 2.29 (s, 3H), 1.52 |
| | | | (s, 9H). |
| 384 | 2-(4-tert-butyl-5- | 404.14 | ¹H NMR (400 MHz, DMSO-d₆) δ 12.23 (s, |
| | chloro-2-methyl- | 405.3 | 1H), 8.72-8.61 (m, 4H), 7.63 (d, J = 5.9 |
| | phenyl)-5-pyrazin-2- | | Hz, 1H), 7.52 (s, 1H), 7.49 (s, 1H), 6.08 (s, |
| | y1-1H-1,6- | | 1H), 2.32 (s, 3H), 1.49 (s, 9H). |
| | naphthyridin-4-one | | |
| 385 | 2-(4-tert-butyl-5- | 417.16 | |
| | chloro-2-methyl- | 418.65 | |
| | phenyl)-5-(6-methyl- | | |
| | 2-pyridyl)-1H-1,6- | | |
| | naphthyridin-4-one | | |

### Example 71

### 2-(4-tert-butyl-5-chloro-2-methyl-phenyl)-5-(4-methyloxazol-5-yl)-1H-1,6-naphthyridin-4-one (386)

### Step 1: 2-(4-tert-butyl-5-chloro-2-methyl-phenyl)-5-(4-methyloxazol-5-yl)-1H-1,6-naphthyridin-4-one (386)

A microwave vial charged with 4-benzyloxy-2-(4-tert-butyl-5-chloro-2-methyl-phenyl)-5-chloro-1,6-naphthyridine (20 mg, 0.04 mmol), 4-methyl-5-(4,4,5,5-tetramethyl-1,3,2-dioxaborolan-2-yl)oxazole (12.3 mg, 0.06 mmol), Pd(PPh₃)₄(8 mg, 0.007 mmol) and sodium carbonate (18 mg, 0.17 mmol) was purged with nitrogen. 1,4-dioxane (400 μL) and water (40 μL) were added followed by sparging with nitrogen (bubbling via syringe) for 10 minutes. The vial was capped, and the reaction mixture was stirred and heated at 80° C. for 1 hour. The reaction was diluted with DMSO (500 uL), filtered, and purified by reverse phase preparative column chromatography (C₁₈) using 1 to 99% MeCN in water (HCl modifier) to give the benzyl intermediate, which was dissolved in ethanol (400 μL) and then added via a syringe to a sealed tube containing 10% palladium on carbon (9 mg, 0.008 mmol). The reaction mixture was stirred under an atmosphere of molecular hydrogen (balloon) for 30 minutes and filtered over abed of celite. The filtrate was concentrated in vacuo. Purification by reverse phase HPLC (C₁₈) using 1 to 99% MeCN in water (HCl modifier) provided 2-(4-tert-butyl-5-chloro-2-methyl-phenyl)-5-(4-methyloxazol-5-yl)-1H-1,6-naphthyridin-4-one (386, 2.2 mg, 12%). ESI-MS m/z calc. 407.14, found 408.2 (M+1)⁺. ¹H NMR (400 MHz, CD₃OD) δ 8.70 (d, J=6.6 Hz, 1H), 8.44 (s, 1H), 7.85 (d, J=6.6 Hz, 1H), 7.55-7.50 (i, 2H), 6.42 (s, 1H), 2.39 (s, 3H), 2.26 (s, 3H), 1.53 (s, 9H).

The following compounds were synthesized using the route shown in Example 71, using the appropriate heterocyclic boronates. Suzuki coupling followed by deprotection provided the following compounds.

TABLE 7

| | | LC/MS | |
|---|---|---|---|
| Cmpd. | | (m/z calc.), | |
| No. | Compound Name | Found M + 1 | NMR (shifts in ppm) |
| 387 | 2-(4-tert-butyl-5- | 406.16 | ¹H NMR (400 MHz, DMSO-d₆) δ |
| | chloro-2-methyl- | 407.2 | 11.99 (s, 1H), 8.57 (d, J = 5.7 Hz, 1H), |
| | phenyl)-5-(4-methyl- | | 7.54-7.41 (m, 3H), 7.35 (s, 1H), 6.14 |
| | 1H-pyrazol-3-yl)-1H- | | (s, 1H), 2.33 (s, 3H), 1.98 (s, 3H), 1.49 |
| | 1,6-naphthyridin-4- | | (s, 9H). |
| | one | | |
| 388 | 2-(4-tert-butyl-5- | 421.17 | |
| | chloro-2-methyl- | 422.55 | |
| | phenyl)-5-(1,5- | | |
| | dimethyltriazol-4-yl)- | | |
| | 1H-1,6-naphthyridin- | | |
| | 4-one | | |
| 389 | 2-(4-tert-butyl-5- | 421.16 | — |
| | chloro-2-methyl- | 422.33 | |
| | phenyl)-5-(2,4- | | |
| | dimethyloxazol-5-yl)- | | |
| | 1H-1,6-naphthyridin- | | |
| | 4-one | | |
| 390 | ethyl 5-[2-(4-tert- | 479.16 | — |
| | butyl-5-chloro-2- | 480.66 | |
| | methyl-phenyl)-4-oxo- | | |
| | 1H-1,6-naphthyridin- | | |
| | 5-yl]-4-methyl- | | |
| | oxazole-2-carboxylate | | |
| 391 | 2-(4-tert-butyl-5- | 437.13 | — |
| | chloro-2-methyl- | 438.29 | |
| | phenyl)-5-(2,5- | | |
| | dimethylthiazol-4-yl)- | | |
| | 1H-1,6-naphthyridin- | | |
| | 4-one | | |
| 392 | 2-(4-tert-butyl-5- | 460.13 | — |
| | chloro-2-methyl- | 461.46 | |
| | phenyl)-5-[1- | | |
| | (trifluoromethyl)pyrazol- | | |
| | 3-yl]-1H-1,6- | | |
| | naphthyridin-4-one | | |

### Example 72

### 2-(4-tert-butyl-5-chloro-2-methyl-phenyl)-5-(3,5-dimethylpyrazol-1-yl)-1H-1,6-naphthyridin-4-one (393)

### Step 1: 2-(4-tert-butyl-5-chloro-2-methyl-phenyl)-5-(3,5-dimethylpyrazol-1-yl)-1H-1,6-naphthyridin-4-one (393)

A solution of 3,5-dimethyl-1H-pyrazole (4 mg, 0.035 mmol), 4-benzyloxy-2-(4-tert-butyl-5-chloro-2-methyl-phenyl)-5-chloro-1,6-naphthyridine (10 mg, 0.02 mmol), and Cs₂CO₃ (25 mg, 0.07 mmol) in DMSO (0.5 mL) was stirred at 90° C. for 2 hours. Purification by reverse phase HPLC (C₁₈) using 1-99% MeCN in water (HCl modifier) provided the benzyl intermediate, which was taken up in toluene (0.5 mL) and treated with TFA (500 μL). The reaction mixture was stirred at 70° C. for 16 hours and concentrated in vacuo. Purification by reverse phase HPLC (C₁₈) using 1-99% MeCN in water (HCl modifier) provided 2-(4-tert-butyl-5-chloro-2-methyl-phenyl)-5-(3,5-dimethylpyrazol-1-yl)-1H-1,6-naphthyridin-4-one (393, 5.4 mg, 58%). ESI-MS m/z calc. 420.17, found 421.2 (M+1)⁺. ¹H NMR (400 MHz, CD₃OD) δ 8.72 (d, J=5.9 Hz, 1H), 7.84 (d, J=5.9 Hz, 1H), 7.52 (s, 1H), 7.50 (s, 1H), 6.60 (s, 1H), 6.38 (s, 1H), 2.51 (s, 3H), 2.37 (s, 3H), 2.31 (s, 3H), 1.52 (s, 9H).

The following compounds were synthesized using the route shown in Example 72, using the appropriate heterocycles. SNAr reaction followed by deprotection provided the following compounds.

TABLE 8

| | | LC/MS | |
|---|---|---|---|
| Cmpd. | | (m/z calc.), | |
| No. | Compound Name | Found M + 1 | NMR (shifts in ppm) |
| 394 | 2-(4-tert-butyl-5- | 460.13 | ¹H NMR (400 MHz, CD₃OD) δ 8.56 (d, |
| | chloro-2-methyl- | 461.3 | J = 5.9 Hz, 1H), 8.16-8.10 (m, 1H), |
| | phenyl)-5-[5- | | 7.67 (d, J = 5.8 Hz, 1H), 7.50 (s, 1H), |
| | (trifluoromethyl)pyrazol- | | 7.49 (s, 1H), 6.83 (d, J = 2.6 Hz, 1H), |
| | 1-yl]-1H-1,6- | | 6.28 (s, 1H), 2.36 (s, 3H), 1.52 (s, 9H). |
| | naphthyridin-4-one | | |
| 395 | 2-(4-tert-butyl-5- | 406.16 | ¹H NMR (400 MHz, CD₃OD) δ 8.58 (d, |
| | chloro-2-methyl- | 407.2 | J = 5.9 Hz, 1H), 8.39 (d, J = 2.7 Hz, |
| | phenyl)-5-(5- | | 1H), 7.64 (d, J = 5.9 Hz, 1H), 7.54 (apps, |
| | methylpyrazol-1-yl)- | | 2H), 6.67 (s, 1H), 6.50 (d, J = 2.7 Hz, |
| | 1H-1,6-naphthyridin- | | 1H), 2.45 (s, 3H), 2.38 (s, 3H), 1.53 (s, |
| | 4-one | | 9H). |

### Example 73

### 2-(4-tert-butyl-5-chloro-2-methyl-phenyl)-5-(1,4-dimethylimidazol-2-yl)-1H-1,6-naphthyridin-4-one (396)

### Step 1: 2-(4-tert-butyl-5-chloro-2-methyl-phenyl)-5-(1,4-dimethylimidazol-2-yl)-1H-1,6-naphthyridin-4-one (396)

A solution of 4-benzyloxy-2-(4-tert-butyl-5-chloro-2-methyl-phenyl)-5-chloro-1,6-naphthyridine (72 mg, 0.16 mmol), dichloropalladium triphenylphosphane (44 mg, 0.06 mmol), Cs₂CO₃ (76 mg, 0.23 mmol), CuI (1.7 mg, 0.009 mmol), and 1,4-dimethylimidazole (10 mg, 0.1 mmol) in dioxane (1.5 mL) was sparged with nitrogen for 5 minutes and then stirred at 100° C. for 16 h. Purification by reverse phase HPLC (C₁₈) using 1-99% MeCN in water (HCl modifier) provided the benzyl intermediate, which was taken up in toluene (0.5 mL) and TFA (500 μL) and heated at 70° C. for 3 h. Purification by reverse phase HPLC (C₁₈) using 1-99% MeCN in water (HCl modifier) provided 2-(4-tert-butyl-5-chloro-2-methyl-phenyl)-5-(1,4-dimethylimidazol-2-yl)-1H-1,6-naphthyridin-4-one (Hydrochloride salt) (396, 2.4 mg, 5%). ESI-MS m/z calc. 420.17, found 421.2 (M+1)⁺. ¹H NMR (400 MHz, CD₃OD) δ 8.85 (d, J=5.9 Hz, 1H), 7.82 (d, J=5.9 Hz, 1H), 7.52 (s, 1H), 7.49 (s, 1H), 7.41 (s, 1H), 6.35 (s, 1H), 3.65 (s, 3H), 2.42 (d, J=1.0 Hz, 3H), 2.36 (s, 3H), 1.52 (s, 9H).

The following compounds were synthesized using the route shown in Example 73, using the appropriate heterocycles. C—H activation followed by deprotection provided the following compounds.

TABLE 9

| | | LC/MS | |
|---|---|---|---|
| | | (m/z calc.), | |
| Table 8. Cmpd. | | Found | |
| No. | Compound Name | M + 1 | NMR (shifts in ppm) |
| 397 | 2-(4-tert-butyl-5- | 407.15 | |
| | chloro-2-methyl- | 408.64 | |
| | phenyl)-5-(4-methyl- | | |
| | 1,2,4-triazol-3-yl)-1H- | | |
| | 1,6-naphthyridin-4- | | |
| | one | | |
| 398 | 2-(4-tert-butyl-5- | 441.11 | |
| | chloro-2-methyl- | 442.37 | |
| | phenyl)-5-(5-chloro-2- | | |
| | methyl-1,2,4-triazol-3- | | |
| | yl)-1H-1,6- | | |
| | naphthyridin-4-one | | |
| 399 | 2-(4-tert-butyl-2,5- | 421.17 | |
| | dimethyl-phenyl)-5- | 422.45 | |
| | (5-chloro-2-methyl- | | |
| | 1,2,4-triazol-3-yl)-1H- | | |
| | 1,6-naphthyridin-4- | | |
| | one | | |
| 400 | 2-(4-tert-butyl-5- | 421.17 | |
| | chloro-2-methyl- | 422.64 | |
| | phenyl)-5-(4,5- | | |
| | dimethyl-1,2,4-triazol- | | |
| | 3-yl)-1H-1,6- | | |
| | naphthyridin-4-one | | |
| 401 | 2-(6-tert-butyl-5- | 421.17 | ¹H NMR (400 MHz, DMSO-d₆) δ |
| | chloro-2-methyl-3- | 422.3 | 12.73 (s, 1H), 8.83 (d, J = 5.8 Hz, 1H), |
| | pyridyl)-5-(1,4- | | 7.95 (s, 1H), 7.91 (d, J = 5.8 Hz, 1H), |
| | dimethylimidazol-2- | | 7.52 (s, 1H), 6.34 (s, 1H), 3.55 (s, 3H), |
| | yl)-1H-1,6- | | 2.51 (s, 3H), 2.35 (s, 3H), 1.50 (s, 9H). |
| | naphthyridin-4-one | | |
| 402 | 2-(6-tert-butyl-5- | 408.15 | ¹H NMR (400 MHz, DMSO-d₆) δ |
| | chloro-2-methyl-3- | 409.3 | 12.73 (s, 1H), 8.83 (d, J = 5.8 Hz, 1H), |
| | pyridyl)-5-(2-methyl- | | 7.95 (s, 1H), 7.91 (d, J = 5.8 Hz, 1H), |
| | 1,2,4-triazol-3-yl)-1H- | | 7.52 (d, J = 1.2 Hz, 1H), 6.34 (s, 1H), |
| | 1,6-naphthyridin-4- | | 3.55 (s, 3H), 2.35 (s, 3H), 1.50 (s, 9H). |
| | one | | |
| 403 | 2-[5-chloro-2-methyl- | 474.14 | ¹H NMR (400 MHz, DMSO-d₆) δ |
| | 4-(2,2,2-trifluoro-1,1- | 475.3 | 12.66 (s, 1H), 8.84 (s, 1H), 7.98-7.83 |
| | dimethyl- | | (m, 1H), 7.82-7.59 (m, 2H), 7.54 (s, |
| | ethyl)phenyl]-5-(1,4- | | 1H), 6.28 (s, 1H), 3.61-3.53 (m, 3H), |
| | dimethylimidazol-2- | | 2.72-2.55 (m, 3H), 2.43-2.32 (m, |
| | yl)-1H-1,6- | | 3H), 1.81 (s, 6H). |
| | naphthyridin-4-one | | |
| 404 | 2-[5-chloro-2-methyl- | 475.14 | ¹H NMR (400 MHz, DMSO-d₆) δ |
| | 4-(2,2,2-trifluoro-1,1- | 476.3 | 12.40 (s, 1H), 8.73 (d, J = 5.7 Hz, 1H), |
| | dimethyl- | | 7.75 (d, J = 5.7 Hz, 1H), 7.67 (s, 1H), |
| | ethyl)phenyl]-5-(2,5- | | 7.61 (s, 1H), 6.18 (s, 1H), 3.56 (s, 3H), |
| | dimethyl-1,2,4-triazol- | | 2.35 (d, J = 1.9 Hz, 6H), 1.80 (s, 6H). |
| | 3-yl)-1H-1,6- | | |
| | naphthyridin-4-one | | |
| 405 | 2-(6-tert-butyl-5- | 422.16 | ¹H NMR (400 MHz, DMSO-d₆) δ |
| | chloro-2-methyl-3- | 423.3 | 12.31 (s, 1H), 8.71 (d, J = 5.8 Hz, 1H), |
| | pyridyl)-5-(2,5- | | 7.96 (s, 1H), 7.69 (d, J = 5.8 Hz, 1H), |
| | dimethyl-1,2,4-triazol- | | 6.24 (s, 1H), 3.52 (s, 3H), 2.49 (s, 3H), |
| | 3-yl)-1H-1,6- | | 2.31 (s, 3H), 1.50 (s, 9H). |
| | naphthyridin-4-one | | |
| 406 | 2-(4-tert-butyl-5- | 457.17 | |
| | chloro-2-methyl- | 458.38 | |
| | phenyl)-5-(1- | | |
| | methylimidazo[4,5- | | |
| | b]pyridin-2-yl)-1H- | | |
| | 1,6-naphthyridin-4- | | |
| | one | | |

### Example 74

### 2-(4-tert-butyl-2,5-dimethyl-phenyl)-5-(1,5-dimethylimidazol-2-yl)-1H-1,6-naphthyridin-4-one (407)

### Step 1: 2-(4-tert-butyl-2,5-dimethyl-phenyl)-5-(1,5-dimethylimidazol-2-yl)-1H-1,6-naphthyridin-4-one (407)

A solution of 4-benzyloxy-2-(4-tert-butyl-5-chloro-2-methyl-phenyl)-5-(5-chloro-1-methyl-imidazol-2-yl)-1,6-naphthyridine (18 mg, 0.03 mmol), K₂CO₃ (15 mg, 0.11 mmol), 2,4,6-trimethyl-1,3,5,2,4,6-trioxatriborinane (30 μL, 0.21 mmol), and RuPhos Pd G3 (9 mg, 0.01 mmol) in dioxane (1 mL) was sparged with nitrogen for 5 minutes and then stirred at 90° C. for 60 minutes. Purification by reverse phase HPLC (C₁₈) using 1-99% MeCN in water (HCl modifier) provided 2-(4-tert-butyl-2,5-dimethyl-phenyl)-5-(1,5-dimethylimidazol-2-yl)-1H-1,6-naphthyridin-4-one (Hydrochloride salt) (407, 2.7 mg, 18%). ESI-MS m/z calc. 400.22, found 401.4 (M+1)⁺. ¹H NMR (400 MHz, CD₃OD) δ 8.84 (d, J=5.9 Hz, 1H), 7.82 (d, J=5.9 Hz, 1H), 7.44 (d, J=1.2 Hz, 1H), 7.40 (s, 1H), 7.21 (s, 1H), 6.33 (s, 1H), 3.58 (s, 3H), 2.58 (s, 3H), 2.47 (s, 3H), 2.34 (s, 3H), 1.45 (s, 9H).

### Example 75

### 2-(4-tert-butyl-5-chloro-2-methyl-phenyl)-5-(1,4-dimethylpyrazol-3-yl)-1H-1,6-naphthyridin-4-one (408)

### Step 1: 2-(4-tert-butyl-5-chloro-2-methyl-phenyl)-5-(1,4-dimethylpyrazol-3-yl)-1H-1,6-naphthyridin-4-one (408)

A DMF (1 mL) mixture of NaH (1.7 mg, 0.04250 mmol) was treated with 4-benzyloxy-2-(4-tert-butyl-5-chloro-2-methyl-phenyl)-5-(4-methyl-1H-pyrazol-3-yl)-1,6-naphthyridine (11 mg, 0.02 mmol) at 0° C. and the reaction was stirred for 30 minutes. Iodomethane (1.4 μL, 0.02249 mmol) was added and the reaction mixture was warmed to room temperature and stirred for 1 hour. The reaction mixture was filtered and the filtrate was purified by reverse phase HPLC (C₁₈) using 1-99% MeCN in water (HCl modifier) to give the benzyl intermediate, which was taken up in toluene (0.5 mL) and TFA (500 μL) and heated at 70° C. for 16 hours. The reaction mixture was concentrated in vacuo and purified by reverse phase HPLC (C₁₈) using 1-99% MeCN in water (HCl modifier) to obtain 2-(4-tert-butyl-5-chloro-2-methyl-phenyl)-5-(1,4-dimethylpyrazol-3-yl)-1H-1,6-naphthyridin-4-one (Hydrochloride salt) (408, 9.4 mg, 91%). ESI-MS m/z calc. 420.17, found 421.2 (M+1)⁺. 1H NMR (400 MHz, DMSO-d6) δ 12.37 (s, 1H), 8.58 (d, J=6.2 Hz, 1H), 7.68 (d, 1H), 7.57 (s, 1H), 7.54 (s, 1H), 7.50 (s, 1H), 6.21 (s, 1H), 3.87 (s, 3H), 2.34 (s, 3H), 1.86 (s, 3H), 1.49 (s, 9H).

### Example 76

### 2-(4-tert-butyl-5-chloro-2-methyl-phenyl)-5-(1,5-dimethylimidazol-2-yl)-1H-1,6-naphthyridin-4-one (409)

### Step 1: 2-(4-tert-butyl-5-chloro-2-methyl-phenyl)-5-(1,5-dimethylimidazol-2-yl)-1H-1,6-naphthyridin-4-one (409)

2-(4-tert-butyl-5-chloro-2-methyl-phenyl)-5-(1,5-dimethylimidazol-2-yl)-1H-1,6-naphthyridin-4-one (409) was prepared using a procedure analogous to that found in Example 74 (Step 1) using 4-benzyloxy-2-(4-tert-butyl-5-chloro-2-methyl-phenyl)-5-(5-chloro-1-methyl-imidazol-2-yl)-1,6-naphthyridine, followed by deprotection using Pd/C if required. ESI-MS m/z calc. 420.17, found 421.732 (M+1)⁺. ¹H NMR (400 MHz, CD₃OD) δ 8.69 (d, J=5.9 Hz, 1H), 7.67 (d, J=5.9 Hz, 1H), 7.50 (s, 1H), 7.47 (s, 1H), 6.96 (s, 1H), 6.26 (s, 1H), 3.41 (s, 3H), 2.35 (s, 6H), 1.52 (s, 9H).

### Example 77

### 2-(4-tert-butyl-5-chloro-2-methyl-phenyl)-5-(2,5-dimethyl-1,2,4-triazol-3-yl)-1H-1,6-naphthyridin-4-one (410)

### Step 1: 2-(4-tert-butyl-5-chloro-2-methyl-phenyl)-5-(2,5-dimethyl-1,2,4-triazol-3-yl)-1H-1,6-naphthyridin-4-one (410)

2-(4-tert-butyl-5-chloro-2-methyl-phenyl)-5-(2,5-dimethyl-1,2,4-triazol-3-yl)-1H-1,6-naphthyridin-4-one (410) was prepared using procedure analogous to that found in Example 74 (Step 1), using 4-benzyloxy-2-(4-tert-butyl-5-chloro-2-methyl-phenyl)-5-(5-chloro-2-methyl-1,2,4-triazol-3-yl)-1,6-naphthyridine, followed by deprotection using Pd/C if required. ESI-MS m/z calc. 421.17, found 422.612 (M+1)⁺. ¹H NMR (400 MHz, CD₃OD) δ 8.73 (d, J=6.0 Hz, 1H), 7.70 (d, J=5.9 Hz, 1H), 7.50 (s, 1H), 7.48 (s, 1H), 6.27 (s, 1H), 3.63 (s, 3H), 2.42 (s, 3H), 2.35 (s, 3H), 1.52 (s, 9H).

### Example 78

### 6-[2-(4-tert-butyl-5-chloro-2-methyl-phenyl)-4-oxo-1H-1,6-naphthyridin-5-yl]pyridine-2-carboxamide (411)

### Step 1: 6-[2-(4-tert-butyl-5-chloro-2-methyl-phenyl)-4-oxo-1H-1,6-naphthyridin-5-yl]pyridine-2-carboxamide (411)

6-[4-benzyloxy-2-(4-tert-butyl-5-chloro-2-methyl-phenyl)-1,6-naphthyridin-5-yl]pyridine-2-carbonitrile (411) was synthesized using a procedure analogous to that found in Example 71, using 6-(4,4,5,5-tetramethyl-1,3,2-dioxaborolan-2-yl)pyridine-2-carbonitrile. ESI-MS m/z calc. 518.18, found 519.622 (M+1)⁺. It was dissolved in toluene (3 mL) and TFA (2 mL) was added and was stirred at 50° C. for 16 hours. Purification via high pressure reverse phase chromatography using 1 to 100% ACN in water containing 5 mM hydrochloric gave 6-[2-(4-tert-butyl-5-chloro-2-methyl-phenyl)-4-oxo-1H-1,6-naphthyridin-5-yl]pyridine-2-carboxamide (412, 10.2 mg, 25%) as a yellow solid. ESI-MS m/z calc. 446.15, found 447.319 (M+1)⁺. ¹H NMR (400 MHz, CD₃OD) δ 8.56 (d, J=5.9 Hz, 1H), 8.16 (dd, J=7.8, 1.0 Hz, 1H), 8.02 (app t, J=7.8 Hz, 1H), 7.67 (dd, J=7.8, 1.0 Hz, 1H), 7.61 (d, J=5.9 Hz, 1H), 7.47 (s, 1H), 7.44 (s, 1H), 6.25 (s, 1H), 2.35 (s, 3H), 1.52 (s, 9H).

### Example 79

### 2-(4-tert-butyl-5-chloro-2-methyl-phenyl)-5-(2-methylpyrimidin-4-yl)-1H-1,6-naphthyridin-4-one (412) & 2-(4-tert-butyl-5-chloro-2-methyl-phenyl)-5-(2-oxo-1H-pyrimidin-4-yl)-1H-1,6-naphthyridin-4-one (413)

### Step 1: 2-(4-tert-butyl-5-chloro-2-methyl-phenyl)-5-(2-methylpyrimidin-4-yl)-1H-1,6-naphthyridin-4-one (412) & 2-(4-tert-butyl-5-chloro-2-methyl-phenyl)-5-(2-oxo-1H-pyrimidin-4-yl)-1H-1,6-naphthyridin-4-one (413)

2-(4-tert-butyl-5-chloro-2-methyl-phenyl)-5-(2-methylpyrimidin-4-yl)-1H-1,6-naphthyridin-4-one (Hydrochloride salt) (412) was prepared using procedure analogous to that found in Example 74, using methylboronic acid, followed by deprotection using TFA/Toluene if required. ESI-MS m/z calc. 418.15, found 419.58 (M+1)⁺. ¹H NMR (400 MHz, CD₃OD) δ 9.00 (d, J=5.4 Hz, 1H), 8.75 (d, J=6.2 Hz, 1H), 7.85 (d, J=5.4 Hz, 1H), 7.82 (d, J=6.2 Hz, 1H), 7.53 (s, 1H), 7.51 (s, 1H), 6.39 (s, 1H), 2.87 (s, 3H), 2.37 (s, 3H), 1.53 (s, 9H).

2-(4-tert-butyl-5-chloro-2-methyl-phenyl)-5-(2-oxo-1H-pyrimidin-4-yl)-1H-1,6-naphthyridin-4-one (413) was also isolated. ESI-MS m/z calc. 420.13, found 421.56 (M+1)⁺. ¹H NMR (400 MHz, CD₃OD) δ 8.65 (d, J=5.9 Hz, 1H), 8.19 (d, J=6.2 Hz, 1H), 7.63 (d, J=5.9 Hz, 1H), 7.50 (s, 1H), 7.48 (s, 1H), 6.66 (d, J=6.2 Hz, 1H), 6.27 (s, 1H), 2.35 (s, 3H), 1.52 (s, 9H).

### Example 80

### 2-(4-tert-butyl-5-chloro-2-methyl-phenyl)-4-oxo-1H-pyrido[2,3-d]pyridazine-5-carboxamide (414)

### Step 1: 4-(4-tert-butyl-5-chloro-2-methyl-phenyl)-2-methoxy-6H-pyrido[2,3-d]pyridazin-5-one

To a solution of 2-chloro-4-methoxy-6H-pyrido[2,3-d]pyridazin-5-one (774 mg, 1.28 mmol) and 4-chloro-2-methoxy-6H-pyrido[2,3-d]pyridazin-5-one (774 mg, 1.28 mmol) in 1,4-dioxane (20 mL) and water (5 mL) was added 2-(4-tert-butyl-5-chloro-2-methyl-phenyl)-4,4,5,5-tetramethyl-1,3,2-dioxaborolane (869 mg, 2.81 mmol) and potassium phosphate tribasic (2.17 g, 10.22 mmol). The reaction mixture was purged with argon gas for 5 minutes then PdCl₂(dtbpf) (167 mg, 0.25 mmol) was added. The reaction was heated at 85° C. overnight. The reaction was cooled to room temperature and partitioned between ethyl acetate (300 mL) and saturated aqueous sodium bicarbonate (150 mL). The organic layer was washed with brine (100 mL), dried over magnesium sulfate, filtered and concentrated. Purification by silica gel column chromatography using 0 to 100% EtOAc in heptane gave 2-(4-tert-butyl-5-chloro-2-methyl-phenyl)-4-methoxy-6H-pyrido[2,3-d]pyridazin-5-one (236 mg, 52%). ¹H-NMR (400 MHz, CDCl₃) δ 9.61 (s, 1H), 8.21 (s, 1H), 7.27 (s, 1H), 7.07 (s, 1H), 6.81 (s, 1H), 4.10 (s, 3H), 2.04 (s, 3H), 1.50 (s, 9H). ESI-MS m/z calc. 357.12, found 358.17 (M+1)⁺. and 4-(4-tert-butyl-5-chloro-2-methyl-phenyl)-2-methoxy-6H-pyrido[2,3-d]pyridazin-5-one (100 mg, 22%). ¹H-NMR (400 MHz, CDCl₃) δ 9.81 (s, 1H), 8.27 (s, 1H), 7.45 (s, 1H), 7.36 (s, 1H), 7.11 (s, 1H), 4.11 (s, 3H), 2.38 (s, 3H), 1.51 (s, 9H). ESI-MS m/z calc. 357.12, found 358.17 (M+1)⁺.

### Step 2: 2-(4-tert-butyl-5-chloro-2-methyl-phenyl)-5-chloro-4-methoxy-pyrido[2,3-d]pyridazine

A mixture of 4-4-tert-butyl-5-chloro-2-methyl-phenyl-2-met oxy-6H-pyrido[2,3-d]pyridazin-5-one (100 mg, 0.28 mmol) and phosphorous oxychloride (8.22 g, 5 mL, 53.64 mmol) was heated at 100° C. for 4 hours with a basic scrubber. The reaction was concentrated under reduced pressure and stirred with ethyl acetate (80 mL) and saturated aqueous sodium hydrogen carbonate solution (30 mL) until the effervescence stopped. The organic layer was dried over magnesium sulfate, filtered and concentrated to obtain 4-(4-tert-butyl-5-chloro-2-methyl-phenyl)-5-chloro-2-methoxy-pyrido[2,3-d]pyridazine (84 mg, 53%). ESI-MS m/z calc. 375.09, found 376.05 (M+1)⁺.

### Step 3: 2-(4-tert-butyl-5-chloro-2-methyl-phenyl)-N-[(2,4-dimethoxyphenyl)methyl]-4-methoxy-pyrido[2,3-d]pyridazine-5-carboxamide

A mixture of 2-(4-tert-butyl-5-chloro-2-methyl-phenyl)-5-chloro-4-methoxy-pyrido[2,3-d]pyridazine (911 mg, 0.88 mmol), DIPEA (371 mg, 500 μL, 2.87 mmol), Pd(dppf)Cl₂ (750 mg, 1 mmol) and (2,4-dimethoxyphenyl)methanamine (780 mg, 4.66 mmol) in 1,4-dioxane (20 mL) was heated at 80° C. under an atmosphere of carbon monoxide (1 atm) for 5 hours. The reaction mixture was cooled to room temperature then partitioned between ethyl acetate (500 mL) and water (250 mL). The organic layer was washed with brine (150 mL) and dried over magnesium sulfate, filtered and concentrated. Purification by silica gel chromatography using 0 to 2.5% MeOH in DCM, followed by reverse phase chromatography (C₁₈) using 30-100% acetonitrile in water (0.1% formic acid) gave 2-(4-tert-butyl-5-chloro-2-methyl-phenyl)-N-[(2,4-dimethoxyphenyl)methyl]-4-methoxy-pyrido[2,3-d]pyridazine-5-carboxamide (210 mg, 40%) as a brown solid. ESI-MS m/z calc. 534.20, found 535.29 (M+1)⁺. ¹H-NMR (400 MHz, CDCl₃) δ 9.68 (s, 1H), 7.45 (s, 1H), 7.40 (d, J=7.8 Hz, 1H), 7.37 (s, 1H), 7.15 (s, 1H), 6.81 (t, J=6.0 Hz, 1H), 6.51-6.48 (m, 2H), 4.69 (d, J=6.0 Hz, 2H), 3.83-3.80 (m, 9H), 2.37 (s, 3H), 1.51 (s, 9H).

### Step 4: 2-(4-tert-butyl-5-chloro-2-methyl-phenyl)-4-methoxy-pyrido[2,3-d]pyridazine-5-carboxamide

A mixture of 2-(4-tert-butyl-5-chloro-2-methyl-phenyl)-N-[(2,4-dimethoxyphenyl)methyl]-4-methoxy-pyrido[2,3-d]pyridazine-5-carboxamide (210 mg, 0.35 mmol) and TFA (14.8 g, 10 mL, 129.8 mmol) was heated at 65° C. for 1 hour. The TFA was removed under reduced pressure and the residue was partitioned between ethyl acetate (300 mL) and saturated aqueous sodium hydrogen carbonate solution (150 mL). The organic phase was washed with brine (75 mL), dried over magnesium sulfate, filtered and concentrated. Purification by reverse phase chromatography (C₁₈) using 25 to 100% acetonitrile in water (0.1% formic acid) gave 2-(4-tert-butyl-5-chloro-2-methyl-phenyl)-4-methoxy-pyrido[2,3-d]pyridazine-5-carboxamide (54 mg, 39%) as a pale cream solid. ¹H-NMR (400 MHz, DMSO-d6) δ 9.64 (s, 1H), 8.03 (s, 1H), 7.78 (s, 1H), 7.66 (s, 1H), 7.61 (s, 1H), 7.44 (s, 1H), 4.08 (s, 3H), 2.38 (s, 3H), 1.47 (s, 9H). ESI-MS m/z calc. 384.13, found 385.18 (M+1)⁺.

### Step 5: 2-(4-tert-butyl-5-chloro-2-methyl-phenyl)-4-oxo-1H-pyrido[2,3-d]pyridazine-5-carboxamide (414)

To a mixture of 2-(4-tert-butyl-5-chloro-2-methyl-phenyl)-4-methoxy-pyrido[2,3-d]pyridazine-5-carboxamide (45 mg, 0.11 mmol) in DCM (10 mL) was added boron tribromide in DCM (1.1 mL of 1 M, 1.1 mmol). The reaction mixture was stirred at room temperature for 4.5 hours and quenched by pouring onto ice. The aqueous layer was extracted with DCM (200 mL×2). The combined organic layer was dried over magnesium sulfate, filtered and concentrated. Purification by reverse phase chromatography (C₁₈) using 15 to 70% acetonitrile in water (0.1% formic acid), followed by silica gel column chromatography using 0 to 5% MeOH in DCM gave 2-(4-tert-butyl-5-chloro-2-methyl-phenyl)-4-oxo-1H-pyrido[2,3-d]pyridazine-5-carboxamide (414, 8.56 mg, 20%). ESI-MS m/z calc. 370.12, found 371.15 (M+1)⁺. ¹H NMR (400 MHz, DMSO-d6) δ 12.51 (br. s, 1H), 9.39 (br. s, 1H), 8.04-7.29 (m, 4H), 6.18 (br. s, 1H), 2.29 (s, 3H), 1.45 (s, 9H).

### Example 81

### 2-(5-tert-butyl-4-chloro-2-methyl-pyrazol-3-yl)-4-oxo-1H-1,6-naphthyridine-5-carboxamide (415)

### Step 1: (5-tert-butyl-2-methyl-pyrazol-3-yl)boronic acid and 4-benzyloxy-2-(5-tert-butyl-2-methyl-pyrazol-3-yl)-1,6-naphthyridine-5-carbonitrile

A mixture of 5-bromo-3-tert-butyl-1-methyl-pyrazole (509 mg, 2.24 mmol), bis(dipinacolato)diboron (880 mg, 3.36 mmol) and potassium acetate (732 mg, 7.38 mmol) in 1,4-dioxane (10 mL) was bubbled with nitrogen for 15 min. Pd(dppf)Cl₂ (225 mg, 0.308 mmol) was added and the mixture heated at 100° C. for 30 min. The mixture was cooled and partitioned between ethyl acetate and water. The organic layer was separated, dried over sodium sulfate, filtered and concentrated to provide (5-tert-butyl-2-methyl-pyrazol-3-yl)boronic acid which was taken directly into the next reaction. ESI-MS m/z calc. 182.12, found 183.1 (M+1)⁺. The boronic acid and 4-benzyloxy-2-chloro-1,6-naphthyridine-5-carbonitrile (440 mg, 1.49 mmol) were combined in dioxane (10 mL) and aqueous tripotassium phosphate (7 mL of 1 M, 7 mmol) and the mixture bubbled with nitrogen for 15 min. PdCl₂(dtbpf) (290 mg, 0.445 mmol) was added and the reaction stirred vigorously under nitrogen for 30 min. The mixture was partitioned between ethyl acetate and water. The organic layer was separated, dried over sodium sulfate, filtered and concentrated. Purification by silica gel chromatography (80 g silica, 0-60% ethyl acetate/hexane over 50 min) provided 4-benzyloxy-2-(5-tert-butyl-2-methyl-pyrazol-3-yl)-1,6-naphthyridine-5-carbonitrile (185 mg, 21%). ESI-MS m/z calc. 397.19, found 398.7 (M+1)⁺.

### Step 2: 4-benzyloxy-2-(5-tert-butyl-4-chloro-2-methyl-pyrazol-3-yl)-1,6-naphthyridine-5-carbonitrile and 2-(5-tert-butyl-4-chloro-2-methyl-pyrazol-3-yl)-4-oxo-1H-1,6-naphthyridine-5-carboxamide (415)

4-Benzyloxy-2-(5-tert-butyl-2-methyl-pyrazol-3-yl)-1,6-naphthyridine-5-carbonitrile (100 mg, 0.252 mmol) and 1-chloropyrrolidine-2,5-dione (67 mg, 0.50 mmol) were combined in acetonitrile (5 mL) and heated at 70° C. under nitrogen in a sealed vial for 1 h. Additional 1-chloropyrrolidine-2,5-dione (67 mg, 0.50 mmol) was added and heating under nitrogen continued for 16 h. The mixture was partitioned between ethyl acetate and water and the layers were separated. The organic layer was dried over sodium sulfate, filtered and concentrated. The residue was dissolved in TFA (2 mL) and toluene (2 mL) and the mixture heated at 75° C. for 16 h. The mixture was concentrated and purified by reverse phase HPLC (Cis, 10-99% acetonitrile/5 mM HCl) to provide 2-(5-tert-butyl-4-chloro-2-methyl-pyrazol-3-yl)-4-oxo-1H-1,6-naphthyridine-5-carboxamide hydrochloride (415, 13.7 mg, 14%). ¹H NMR (400 MHz, CD₃OD) δ 8.80 (d, J=6.3 Hz, 1H), 7.93 (d, J=6.2 Hz, 1H), 6.91 (s, 1H), 3.89 (s, 3H), 1.43 (s, 9H). ESI-MS m/z calc. 359.11, found 360.3 (M+1)⁺.

The following compounds were synthesized using the general route shown in the below general scheme:

### General Scheme

Pyrazole boronates were synthesized from the appropriately alkylated pyrazole. Suzuki coupling followed by the appropriate halogenation and/or deprotection/nitrile hydrolysis provided the following compounds.

TABLE 10

| Com- | | MW & | |
|---|---|---|---|
| pound | | Found | |
| No. | Compound Name | [M + H]⁺ | NMR (shifts in ppm) |
| 416 | 2-(5-tert-butyl-4-chloro- | 387.86; | ¹H NMR (400 MHz, DMSO- |
| | 2-isopropyl-pyrazol-3- | 388.3 | d₆) δ 12.42 (s, 1H), 8.57 (d, |
| | yl)-4-oxo-1H-1,6- | | J = 5.8 Hz, 1H), 7.93-7.23 (m, |
| | naphthyridine-5- | | 3H), 6.34 (s, 1H), 4.62-4.24 |
| | carboxamide | | (m, 1H), 1.39 (s, 9H), 1.37 (d, |
| | | | J = 6.5 Hz, 6H). |
| 417 | 2-(5-tert-butyl-2-methyl- | 299.34; | ¹H NMR (400 MHz, DMSO- |
| | pyrazol-3-yl)-6-fluoro- | 300.5 | d₆) δ 7.84 (dd, J = 9.1, 4.8 Hz, |
| | 1H-quinolin-4-one | | 1H), 7.81-7.74 (m, 1H), 7.66 |
| | | | (td, J = 8.7, 3.0 Hz, 1H), 6.64 |
| | | | (s, 1H), 6.44 (s, 1H), 3.92 (s, |
| | | | 3H), 1.30 (s, 9H). |
| 418 | 2-(5-tert-butyl-2-methyl- | 325.37; | ¹H NMR (400 MHz, CD₃OD) |
| | pyrazol-3-yl)-4-oxo-1H- | 326.5 | δ 8.85 (d, J = 6.1 Hz, 1H), |
| | 1,6-naphthyridine-5- | | 8.08 (d, J = 6.1 Hz, 1H), 7.10 |
| | carboxamide | | (s, 1H), 6.82 (s, 1H), 4.04 (s, |
| | | | 3H), 1.37 (s, 9H). |

### Example 82

### methyl 2-(4-(tert-butyl)-2-methylphenyl)-4-oxo-1,4-dihydro-1,6-naphthyridine-3-carboxylate (419)

methyl 2-(4-(tert-butyl)-2-methylphenyl)-4-oxo-1,4-dihydro-1,6-naphthyridine-3-carboxylate (419) was prepared via a process analogous to that found in Example 4 (Method C). ESI-MS m/z calc. 350.16, found 351 (M+1)⁺. ¹H NMR (400 MHz, CD₃OD) δ 9.40 (s, 1H), 8.64 (s, 1H), 7.46 (d, J=5.9 Hz, 1H), 7.42 (d, J=2.3 Hz, 1H), 7.37 (dd, J=8.0, 2.1 Hz, 1H), 7.26 (d, J=8.1 Hz, 1H), 3.52 (s, 3H), 2.29 (s, 3H), 1.35 (s, 9H).

### Example 83

### 2-(4-(tert-butyl)-5-chloro-2-methylphenyl)-5-(1-methyl-1H-1,2,4-triazol-5-yl)-1,6-naphthyridin-4(1H)-one (420)

2-(4-(tert-butyl)-5-chloro-2-methylphenyl)-5-(1-methyl-1H-1,2,4-triazol-5-yl)-1,6-naphthyridin-4(1H)-one (420) was prepared via a process analogous to that found in Example 70. ESI-MS m/z calc. 407.15, found 408.25 (M+1)⁺. ¹H NMR (400 MHz, CD₃OD) δ 9.40 (s, 1H), 8.64 (s, 1H), 7.46 (d, J=5.9 Hz, 1H), 7.42 (d, J=2.3 Hz, 1H), 7.37 (dd, J=8.0, 2.1 Hz, 1H), 7.26 (d, J=8.1 Hz, 1H), 3.52 (s, 3H), 2.29 (s, 3H), 1.35 (s, 9H).

### Example 84

### rel-(S)-2-(5-chloro-4-(3,3-difluoro-1-methylcyclopentyl)-2-methylphenyl)-4-oxo-1,4-dihydro-1,6-naphthyridine-5-carboxamide (421) & rel-(R)-2-(5-chloro-4-(3,3-difluoro-1-methylcyclopentyl)-2-methylphenyl)-4-oxo-1,4-dihydro-1,6-naphthyridine-5-carboxamide (422)

rac-2-(5-chloro-4-(3,3-difluoro-1-methylcyclopentyl)-2-methylphenyl)-4-oxo-1,4-dihydro-1,6-naphthyridine-5-carboxamide was prepared via a process analogous to that found in Example 4 (Method C). It was subjected to SFC separation using Phenomenex Lux Column Amylose 1 (250×30 mm), 5 μM column at 40° C., eluent: 20% EtOH (0.1% DEA), 80% CO2, flow rate: 100 mL/min, concentration: 12 mg/mL in methanol (no modifier), injection volume: 1500 μL, pressure: 100 bar, wavelength: 210 nm. Retention times of enantiomers were determined using this method (22 min run time).

Peak 1 (421): rel-(S)-2-[5-chloro-4-(3,3-difluoro-1-methyl-cyclopentyl)-2-methyl-phenyl]-4-oxo-1H-1,6-naphthyridine-5-carboxamide. ESI-MS m/z calc. 350.16, found 351 (M+1)⁺. Retention time: 9.883. ¹H NMR (400 MHz, CDCl₃) δ 15.51 (s, 1H), 8.98 (br s, 1H), 8.65 (d, J=5.6 Hz, 1H), 8.13 (d, J=5.6 Hz, 1H), 7.55 (s, 1H), 7.21 (s, 1H), 7.18 (s, 1H), 6.19 (br s, 1H), 2.83 (td, J=15.5, 8.6 Hz, 1H), 2.66-2.50 (m, 1H), 2.41 (s, 3H), 2.39-2.26 (m, 4H), 1.52 (s, 3H).

Peak 2 (422): rel-(R)-2-[5-chloro-4-(3,3-difluoro-1-methyl-cyclopentyl)-2-methyl-phenyl]-4-oxo-1H-1,6-naphthyridine-5-carboxamide. ESI-MS m/z calc. 350.16, found 351 (M+1)⁺. Retention time: 12.850. ¹H NMR (400 MHz, CDCl₃) δ 15.52 (br s, 1H), 8.98 (br s, 1H), 8.65 (d, J=5.4 Hz, 1H), 8.14 (br d, J=5.4 Hz, 1H), 7.55 (s, 1H), 7.21 (s, 1H), 7.18 (s, 1H), 6.22 (br s, 1H), 2.83 (td, J=15.2, 8.7 Hz, 1H), 2.66-2.50 (m, 1H), 2.41 (s, 3H), 2.39-2.26 (m, 4H), 1.52 (s, 3H).

### Example 85

### rel-(R)-2-(5-chloro-6-(3,3-difluoro-1-methylcyclopentyl)-2-methylpyridin-3-yl)-4-oxo-1,4-dihydro-1,6-naphthyridine-5-carboxamide (423) & rel-(S)-2-(5-chloro-6-(3,3-difluoro-1-methylcyclopentyl)-2-methylpyridin-3-yl)-4-oxo-1,4-dihydro-1,6-naphthyridine-5-carboxamide (424)

### Step 1: SFC Separation: rac-4-benzyloxy-2-[5-chloro-6-(3,3-difluoro-1-methyl-cyclopentyl)-2-methyl-3-pyridyl]-1,6-naphthyridine-5-carbonitrile

rac-4-benzyloxy-2-[5-chloro-6-(3,3-difluoro-1-methyl-cyclopentyl)-2-methyl-3-pyridyl]-1,6-naphthyridine-5-carbonitrile was subjected to SFC separation using Diacel ChiralCe OJ-H (250×20 mm), 5 μM column at 35° C., eluent: 12% iPOH (0.1% DEA), 88% CO2, flow rate: 60 mL/min, concentration: 8 mg/mL in ethanol (no modifier), injection volume: 1000 μL, pressure: 100 bar, wavelength: 220 nm. Retention times of enantiomers were determined by SFC using Diacel ChiralCe OJ-H (4.6×250 mm) at ambient temperature, mobile phase of 20% iPOH (0.1% DEA) with CO2, flow rate 2 mL/min, 15 min run time.

Peak 1: rel-(R)-4-benzyloxy-2-[5-chloro-6-(3,3-difluoro-1-methyl-cyclopentyl)-2-methyl-3-pyridyl]-1,6-naphthyridine-5-carbonitrile. Retention time: 8.315 minutes.

Peak 2: rel-(S)-4-benzyloxy-2-[5-chloro-6-(3,3-difluoro-1-methyl-cyclopentyl)-2-methyl-3-pyridyl]-1,6-naphthyridine-5-carbonitrile. Retention time: 9.059 minutes.

### Step 2a: rel-(R)-2-[5-chloro-6-(3,3-difluoro-1-methyl-cyclopentyl)-2-methyl-3-pyridyl]-4-oxo-1H-1,6-naphthyridine-5-carboxamide (423)

rel-(R)-2-[5-chloro-6-(3,3-difluoro-1-methyl-cyclopentyl)-2-methyl-3-pyridyl]-4-oxo-1H-1,6-naphthyridine-5-carboxamide (423) was prepared from rel-(R)-4-Benzyloxy-2-[5-chloro-6-(3,3-difluoro-1-methyl-cyclopentyl)-2-methyl-3-pyridyl]-1,6-naphthyridine-5-carbonitrile (Peak 1), using procedure analogous to that found in Example 4 (Method D, Step 2). ESI-MS m/z calc. 432.12, found 433.3 (M+1)⁺; Retention time: 1.79 minutes. ¹H NMR (400 MHz, CD₃OD) δ 8.56 (d, J=5.9 Hz, 1H), 7.92 (s, 1H), 7.53 (s, 1H), 6.37 (s, 1H), 3.02 (dt, J=18.1, 14.3 Hz, 1H), 2.73 (tt, J=11.4, 6.2 Hz, 1H), 2.54 (s, 3H), 2.52-2.40 (m, 1H), 2.22 (dtd, J=19.8, 15.6, 9.6 Hz, 3H), 1.57 (s, 3H). ¹⁹F NMR (376 MHz, CD₃OD) δ −87.86-−88.11 (m), −88.46-−88.78 (m), −89.31-−89.60 (m), −89.96-−90.18 (m).

### Step 2b: rel-(S)-2-[5-chloro-6-(3,3-difluoro-1-methyl-cyclopentyl)-2-methyl-3-pyridyl]-4-oxo-1H-1,6-naphthyridine-5-carboxamide (424)

rel-(S)-2-[5-chloro-6-(3,3-difluoro-1-methyl-cyclopentyl)-2-methyl-3-pyridyl]-4-oxo-1H-1,6-naphthyridine-5-carboxamide (424) was prepared from rel-(S)-4-Benzyloxy-2-[5-chloro-6-(3,3-difluoro-1-methyl-cyclopentyl)-2-methyl-3-pyridyl]-1,6-naphthyridine-5-carbonitrile (Peak 2), using procedure analogous to that found in Example 4 (Method D, Step 2). ESI-MS m/z calc. 432.12, found 433.4 (M+1)⁺; Retention time: 1.79 minutes. 1H NMR (400 MHz, CD₃OD) δ 8.55 (d, J=5.9 Hz, 1H), 7.92 (s, 1H), 7.53 (d, J=5.9 Hz, 1H), 6.38 (s, 1H), 3.10-2.94 (m, 1H), 2.79-2.68 (m, 1H), 2.54 (s, 3H), 2.53-2.42 (m, 1H), 2.35-2.10 (m, 3H), 1.57 (s, 3H). 19F NMR (376 MHz, CD₃OD) δ −87.80-−88.05 (m), −88.42-−88.71 (m), −89.26-−89.60 (m), −89.89-−90.19 (m).

### Example 86

### 2-(5-chloro-2-methyl-4-(2-(methyl-d₃)propan-2-yl-1,1,1,3,3,3-d₆)phenyl-3-d)-4-oxo-1,4-dihydro-1,6-naphthyridine-5-carboxamide (425)

2-(5-chloro-2-methyl-4-(2-(methyl-d3)propan-2-yl-1,1,1,3,3,3-d6)phenyl-3-d)-4-oxo-1,4-dihydro-1,6-naphthyridine-5-carboxamide (425) was prepared via a process analogous to that found in Example 4 (Method C). ¹H NMR (400 MHz, DMSO-d₆) δ 11.97 (s, 1H), 8.50 (d, J=8 Hz, 1H), 7.52 (bs, 1H), 7.44 (m, 2H), 7.30 (bs, 1H), 6.12 (s, 1H), 2.30 (s, 3H).

### Example 87

### 2-(5-chloro-2-methyl-4-(1,1,1-trifluoro-2-methylpropan-2-yl-3-¹³C-3,3,3-d₃)phenyl-3,6-d₂)-4-oxo-1,4-dihydro-1,6-naphthyridine-5-carboxamide (426)

2-(5-chloro-2-methyl-4-(1,1,1-trifluoro-2-methylpropan-2-yl-3-1³C-3,3,3-d₃)phenyl-3,6-d₂)-4-oxo-1,4-dihydro-1,6-naphthyridine-5-carboxamide (426) was prepared via a process analogous to that found in Example 4 (Method C). ¹H NMR (400 MHz, DMSO-d₆): δ 12.01 (s, 1H), 8.49 (d, J 5.6 Hz, 1H), 7.52 (br, 1H), 7.43 (d, J 5.6 Hz, 1H), 7.30 (br, 1H), 6.14 (s, 1H), 2.31 (s, 3H), 1.78 (m, 3H). ¹⁹F NMR (376 MHz, DMSO-d₆): δ −73.00 (s).

### Example 88

### 2-(5-chloro-4-(1-hydroxy-2-(methyl-d3)propan-2-yl-1,1,3,3,3-d5)-2-methylphenyl)-4-oxo-1,4-dihydro-1,6-naphthyridine-5-carboxamide (427)

2-(5-chloro-4-(1-hydroxy-2-(methyl-d3)propan-2-yl-1,1,3,3,3-d5)-2-methylphenyl)-4-oxo-1,4-dihydro-1,6-naphthyridine-5-carboxamide (427) was prepared via a process analogous to that found in Example 4 (Method C). ¹H NMR (400 MHz, D₂O) δ 8.56 (d, J=4.8 Hz, 1H), 7.63 (d, J=5.6 Hz, 1H), 7.49 (d, J=9.2 Hz, 2H), 6.44 (s, 1H), 2.30 (s, 3H).

### Example 89

### 2-(4-(tert-butyl)-5-chloro-2-methylphenyl)-4-oxo-1,4-dihydro-1,6-naphthyridine-5-sulfonamide

2-(4-(tert-butyl)-5-chloro-2-methylphenyl)-4-oxo-1,4-dihydro-1,6-naphthyridine-5-sulfonamide can be prepared according to the methods disclosed herein.

### Example 90

### E-VIPR Assay Detecting and Measuring Na_{V} Inhibition Properties

Sodium ion channels are voltage-dependent proteins that can be activated by inducing membrane voltage changes by applying electric fields. The electrical stimulation instrument and methods of use, referred to as E-VIPR, are described in International Publication No. WO 2002/008748 A3 and C.-J. Huang et al. Characterization of voltage-gated sodium channel blockers by electrical stimulation and fluorescence detection of membrane potential, 24 Nature Biotech. 439-46 (2006), both of which are incorporated by reference in their entirety. The instrument comprises a microtiter plate handler, an optical system for exciting the coumarin dye while simultaneously recording the coumarin and oxonol emissions, a waveform generator, a current- or voltage-controlled amplifier, and parallel electrode pairs that are inserted into assay plate wells. Under integrated computer control, this instrument passes user-programmed electrical stimulus protocols to cells within the wells of the microtiter plate.

16-20 hours prior to running the assay on E-VIPR, HEK cells expressing a truncated form of human Na_{V} 1.8 with full channel activity were seeded into microtiter 384-well plates, pre-coated with matrigel, at a density of 25,000 cells per well. 2.5-5% KIR2.1 BacMam virus was added to the final cell suspension before seeding into cell plates. HEK cells were grown in Dulbecco's Modified Eagle's Medium (DMEM) supplemented with 10% FBS (Fetal Bovine Serum, qualified; Sigma #F4135), 1% NEAA (Non-Essential Amino Acids, Gibco #11140), 1% HEPES (Gibco #15630), 1% Pen-Strep (Penicillin-Streptomycin; Gibco #15140) and 5 μg/ml Blasticidin (Gibco #R210-01). Cells were expanded in 5-layer CellSTACK culture chambers or cell culture flasks with vented caps, with 90-95% humidity and 5% CO₂.

Reagents and Stock Solutions:

100 mg/mL Pluronic F-127 (Sigma #P2443), in dry DMSO

Compound Plates: Corning 384-well Polypropylene Round Bottom #3656

Cell Plates: 384-well tissue culture treated plates (Greiner #781091-2B)

2.5-5% KIR 2.1 Bacmam virus (produced in-house), prepared as described in Section 3.3 of J. A. Fornwald et al., Gene Expression in Mammalian Cells Using BacMam, a Modified Baculovirus System, 1350 Methods in Molecular Biology 95-116 (2016), the entire contents of which are incorporated by reference. The concentration used can be dependent on viral titer of each batch.

5 mM DiSBAC₆(3), a voltage sensitive oxonol acceptor (CAS number 169211-44-3; 5-[3-(1,3-dihexylhexahydro-4,6-dioxo-2-thioxo-5-pyrimidinyl)-2-propen-1-ylidene]-1,3-dihexyldihydro-2-thioxo-4,6(1H,5H)-pyrimidinedione), in dry DMSO. The preparation of DiSBAC₆(3) is analogous to that of DiSBAC₄(3) as described in Voltage Sensing by Fluorescence Resonance Energy Transfer in Single Cells, Gonzalez, J. E. and Tsien, R. Y. (1995) Biophys. J. 69, 1272-1280.

5 mM CC2-DMPE, a commercially available membrane-bound coumarin phospholipid FRET donor (ThermoFisher Scientific catalog number K1017, CAS number 393782-57-5; tetradecanoic acid, 1,1′-[(1R)-1-[8-(6-chloro-7-hydroxy-2-oxo-2H-1-benzopyran-3-yl)-3-hydroxy-3-oxido-8-oxo-2,4-dioxa-7-aza-3-phosphaoct-1-yl]-1,2-ethanediyl] ester) was prepared in dry DMSO. See also, Improved indicators of cell membrane potential that use fluorescence resonance energy transfer, Gonzalez, J. E. and Tsien, R. Y. (1997) Chem. Biol. 4, 269-277.

Voltage Assay Background Suppression Compound (VABSC-1) is prepared in H₂O (89-363 mM, range used to maintain solubility)

Human Serum (HS, Millipore #S1P1-01KL, or Sigma SLBR5469V and SLBR5470V as a 50%/50% mixture, for 25% assay final concentration)

Bath 1 Buffer:
Sodium Chloride 160 mM (9.35 g/L), Potassium Chloride, 4.5 mM (0.335 g/L), Glucose 10 mM (1.8 g/L), Magnesium Chloride (Anhydrous) 1 mM (0.095 g/L), Calcium Chloride 2 mM (0.222 g/L), HEPES 10 mM (2.38 g/L) in water.

Na/TMA Cl Bath 1 Buffer:
Sodium Chloride 96 mM (5.61 g/L), Potassium Chloride 4.5 mM (0.335 g/L), Tetramethylammonium (TMA)-Cl 64 mM (7.01 g/L), Glucose 10 mM (1.8 g/L), Magnesium Chloride (Anhydrous) 1 mM (0.095 g/L), Calcium Chloride 2 mM (0.222 g/L) HEPES 10 mM (2.38 g/L) in water.

Hexyl Dye Solution (2× concentration):
Bath 1 Buffer containing 0.5% β-cyclodextrin (made fresh prior to each use, Sigma #C4767), 8 μM CC2-DMPE and 2 μM DiSBAC₆(3). The solution was made by adding 10% Pluronic F127 stock equal to combined volumes of CC2-DMPE and DiSBAC₆(3). The order of preparation was first mix Pluronic and CC2-DMPE, then add DiSBAC₆(3), then while vortexing add Bath 1/β-Cyclodextrin.

Compound Loading Buffer (2× concentration): Na/TMA Cl Bath1 Buffer containing HS (omitted in experiments run in the absence of human serum (HS)) 50%, VABSC-1 1 mM, BSA 0.2 mg/ml (in Bath-1), KCl 9 mM, DMSO 0.625%.

Assay Protocol (7 Key Steps):

1) To reach the final concentration in each well, 375 nL of each compound was pre-spotted (in neat DMSO) into polypropylene compound plates at 240× desired final concentration from an intermediate stock concentration of 0.075 mM, in an 11-point dose response, 3-fold dilution, resulting in a top dose of 300 nM final concentration in the cell plate. Vehicle control (neat DMSO), and positive control (an established Na_{V}1.8 inhibitor, 25 μM final in assay in DMSO) were added manually to the outermost columns of each plate respectively. The compound plate was backfilled with 45 μL per well of Compound Loading Buffer resulting in a 240-fold dilution of compound following a 1:1 transfer of compound into the cell plate (see Step 6). Final DMSO concentration for all wells in the assay was 0.625% (0.75% DMSO was supplemented to the Compound Loading Buffer for a final DMSO concentration of 0.625%). This assay dilution protocol was adjusted to enable a higher dose range to be tested in the presence of HS or if the final assay volume was altered.

2) Hexyl Dye Solution was prepared.

3) Cell plates were prepared. On the day of the assay, the media was aspirated, and the cells were washed three times with 80 μL of Bath-1 buffer, maintaining 25 μL residual volume in each well.

4) 25 μL per well of Hexyl Dye Solution was dispensed into the cell plates. The cells were incubated for 20 minutes at room temperature or ambient conditions in darkness.

5) 45 μL per well of Compound Loading Buffer was dispensed into compound plates.

6) The cell plates were washed three times with 80 μL per well of Bath-1 Buffer, leaving 25 L of residual volume. Then 25 μL per well from compound plate was transferred to each cell plate. The mixture was incubated for 30 minutes at room temperature/ambient conditions.

7) The cell plate containing compound was read on E-VIPR using the current-controlled amplifier to deliver stimulation wave pulses using a symmetrical biphasic waveform. The user-programmed electrical stimulus protocols were 1.25-4 Amps and 4 millisecond pulse width (dependent on electrode composition) were delivered at 10 Hz for 10 seconds. A pre-stimulus recording was performed for each well for 0.5 seconds to obtain the un-stimulated intensities baseline. The stimulatory waveform was followed by 0.5 seconds of post-stimulation recording to examine the relaxation to the resting state. All E-VIPR responses were measured at 200 Hz acquisition rate.

Data Analysis:

Data were analyzed and reported as normalized ratios of emission intensities measured in the 460 nm and 580 nm channels. The response as a function of time was reported as the ratios obtained using the following formula:

$$R ( t ) = \frac{( {intensity}_{460 n m} )}{( {intensity}_{580 n m} )}$$

The data were normalized by calculating the initial (Rᵢ) and final (R_{f}) ratios. These were the average ratio values during part or all of the pre-stimulation period and during sample points during the stimulation period. The fluorescence ratio (R_{f}/Rᵢ) was then calculated and reported as a function of time.

Control responses were obtained by performing assays in the presence of the positive control, and in the absence of pharmacological agents (DMSO vehicle negative control). Responses to the negative (N) and positive (P) controls were calculated as above. The compound antagonist % activity A was then defined as:

$$A = \frac{X - N}{P - N} \times 100$$

where X is the maximum amplitude of the ratio response or number of action potential peaks, at the beginning of the pulse train in the presence of test compound. Using this analysis protocol, dose response curves were plotted and IC₅₀ values were generated for various compounds of the present invention.

Compounds having a measured IC₅₀ value less than 0.5 μM in the E-VIPR Assay described above include: 4, 14, 38, 55, 80, 85, 87-90, 92-103, 105, 108-110, 112, 114-116, 119-122, 125, 127-129, 131-137, 139-160, 162-164, 167-176, 178, 179, 181-185, 187-193, 196, 199, 201-207, 210, 211, 213-220, 222, 223, 225-227, 230, 232, 233, 236-238, 240-245, 248, 250-272, 274-285, 287-294, 298, 299, 301-303, 306-314, 316-327, 329-334, 336, 338-345, 348-350, 354, 357, 358, 360-363, 367-374, 376-389, 391-416, and 419-425.

Compounds having a measured IC₅₀ value less than 2 μM and greater than or equal to 0.5 μM in the E-VIPR Assay described above include: 5, 7, 8, 12, 54, 74, 107, 117, 118, 123, 124, 126, 180, 195, 200, 208, 209, 212, 221, 224, 228, 231, 234, 239, 246, 247, 273, 286, 304, 337, 353, 365, 366, 390, and 417.

Compounds having a measured IC₅₀ value less than 5 μM and greater than or equal to 2 μM in the E-VIPR Assay described above include: 6, 17, 19, 23, 28, 52, 70, 72, 75, 111, 138, 166, 297, 346, 347, 351, 352, 356, 359, and 427.

Compounds having a measured IC₅₀ value greater than or equal to 5 μM in the E-VIPR Assay described above include: 1-3, 9-11, 13, 15, 16, 18, 20-22, 24-27, 29-37, 39-51, 53, 57-69, 71, 73, 76-79, 81-84, 86, 91, 104, 106, 130, 186, 194, 197, 198, 229, 235, 249, 295, 300, 305, 315, 328, 335, 355, 364, 375, and 418.

An IC₅₀ value was not determined in the E-VIPR Assay described for Compounds 56, 113, 161, 165, 177, 296, and 426.

Many modifications and variations of the embodiments described herein may be made without departing from the scope, as is apparent to those skilled in the art. The specific embodiments described herein are offered by way of example only.

1-122. (canceled)

123. A compound of formula (I-E):
or a pharmaceutically acceptable salt thereof, wherein:
X₅ₐ is N or N⁺—O⁻;
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —NHC(O)NH₂, —NHC(O)NH(C₁-C₆ alkyl), —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′};
R^{9a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
each R^{a′} is independently halo, —OH, C₁-C₆ alkyl, C₁-C₆ haloalkyl, —C(O)O(C₁-C₆ alkyl), or —C(O)NH₂;
X_{3b} is N or CR^{3b};
X_{6b} is N or CR^{6b};
R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{3b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ haloalkylene)-OH, —(C₁-C₆ haloalkylene)-C(O)OH, —C(O)(C₁-C₆ alkyl), —C(O)(C₁-C₆ haloalkyl), —C(O)(O)(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′};
R^{5b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{6b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
each R^{b′} is independently C₁-C₆ alkyl or C₁-C₆ haloalkyl,
provided that if X_{3b} is CR^{3b} and X_{6b} is CR^{6b}, then no more than three of R^{2b}, R^{3b}, R^{4b}, R^{5b}, and R^{6b} are H.

124. The compound of claim 123, or a pharmaceutically acceptable salt thereof, wherein:
R^{6a} is H, halo, —CN, C₁-C₆ alkyl, C₁-C₆ alkenyl, C₁-C₆ haloalkyl, C₁-C₆ haloalkenyl, C₁-C₄ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
R^{4b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo.

125. The compound of claim 123, wherein the compound is of formula (I-F):
or a pharmaceutically acceptable salt thereof, wherein:
X_{3b} is N or CH;
R^{2b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{4b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ haloalkylene)-OH, —(C₁-C₆ haloalkylene)-C(O)OH, —C(O)(C₁-C₆ alkyl), —C(O)(C₁-C₆ haloalkyl), —C(O)(O)(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′};
R^{5b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
each R^{b′} is independently C₁-C₆ alkyl or C₁-C₆ haloalkyl.

126. The compound of claim 123, wherein the compound is of formula (I-G):
or a pharmaceutically acceptable salt thereof, wherein:
X_{3b} is N or CH;
R^{2b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{4b} is halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ haloalkylene)-OH, —(C₁-C₆ haloalkylene)-C(O)OH, —C(O)(C₁-C₆ alkyl), —C(O)(C₁-C₆ haloalkyl), —C(O)(O)(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and
each R^{b′} is independently C₁-C₆ alkyl or C₁-C₆ haloalkyl.

127. The compound of claim 123, or a pharmaceutically acceptable salt thereof, wherein X₅ₐ is N.

128. The compound of claim 123, or a pharmaceutically acceptable salt thereof, wherein X₅ₐ is N⁺—O⁻.

129. The compound of claim 123, or a pharmaceutically acceptable salt thereof, wherein R^{9a} is H, halo, C₁-C₆ alkyl, C₁-C₆ alkoxy, —C(O)OH, or —C(O)O(C₁-C₆ alkyl);
optionally wherein R^{9a} is H, Cl, Br, —CH₃, —OCH₃, —OCH₂CH₃, —C(O)OH, or —C(O)OCH₃.

130. The compound of claim 123, or a pharmaceutically acceptable salt thereof, wherein R^{6a} is H, —CN, C₁-C₆ alkoxy, —NH(C₁-C₆ alkyl), —N(C₁-C₆ alkyl)₂, —NHC(O)NH₂, —NHC(O)NH(C₁-C₆ alkyl), —OH, —O(C₁-C₆ alkylene)-(C₁-C₆ alkoxy), —O(C₁-C₆ alkylene)-N(C₁-C₆ alkyl)₂, —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′}; and
each R^{a′} is independently Cl, —OH, —CH₃, —CH₂CH₃, —CF₃, —C(O)OCH₂CH₃, or —C(O)NH₂;
optionally wherein:
R^{6a} is H, —CN, —OCH₃, —OCH₂CH₂CH(CH₃)₂, —NH(CH₃), —N(CH₃)₂, —NHC(O)NH₂, —NHC(O)NHCH₃, —OH, —OCH₂CH₂OCH₃, —OCH₂CH₂N(CH₃)₂, —C(O)NH₂, —C(O)NH(CH₃), —C(O)N(CH₃)₂, —C(O)OH, —C(O)OCH₃, —C(NH)NH₂, —C(S)NH₂, —SO₂NH₂,

131. The compound of claim 123, or a pharmaceutically acceptable salt thereof, wherein X_{3b} is N.

132. The compound of claim 123, or a pharmaceutically acceptable salt thereof, wherein X_{3b} is CR^{3b} and R^{3b} is H, halo, C₁-C₆ alkyl, or C₁-C₆ haloalkyl;
optionally wherein R^{3b} is H, F, —CH₃ or —CF₃.

133. The compound of claim 123, or a pharmaceutically acceptable salt thereof, wherein X_{6b} is N.

134. The compound of claim 123, or a pharmaceutically acceptable salt thereof, wherein X_{6b} is CR^{6b} and R^{6b} is H, halo, or C₁-C₆ alkyl;
optionally wherein R^{6b} is H, F, or —CH₃.

135. The compound of claim 123, or a pharmaceutically acceptable salt thereof, wherein R^{2b} is H, halo, C₁-C₆ alkyl, C₁-C₆ alkoxy, —(C₁-C₆ alkylene)-OH, or —C(O)O(C₁-C₆ alkyl);
optionally wherein R^{2b} is H, F, Cl, —CH₃, —CH₂CH₃, —OCH₃, —CH₂OH, —CH₂CH₂CH₂OH, or —C(O)OCH₃.

136. The compound of claim 123, or a pharmaceutically acceptable salt thereof, wherein R^{4b} is H, halo, C₁-C₆ alkyl, C₁-C₆ alkenyl, C₁-C₆ haloalkyl, C₁-C₆ haloalkenyl, C₁-C₆ haloalkoxy, —(C₁-C₆ alkylene)-OH, —(C₁-C₆ haloalkylene)-OH, —(C₁-C₆ haloalkylene)-C(O)OH, —C(O)O(C₁-C₆ alkyl), —Si(C₁-C₆ alkyl)₃, C₆-C₁₀ aryl, C₃-C₁₀ cycloalkyl, (C₁-C₆ alkyl)-(C₃-C₆ cycloalkyl)-, (C₁-C₆ haloalkyl)-(C₃-C₆ cycloalkyl)-, or 4-10 membered heterocyclyl comprising 1-3 heteroatoms selected from nitrogen and oxygen, wherein cycloalkyl in said C₃-C₁₀ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo and said heterocyclyl is optionally substituted with 1-2 R^{b′}; and
each R^{b′} is independently —CH₃ or —CF₃;
optionally wherein:
R^{4b} is H, Cl, —CH₃, —CH(CH₃)₂, —C(CH₃)₃, —C(CH₃)₂(CH₂CH₃), —CF₃, —C(CH₃)₂(CHF₂), —C(CH₃)₂(CF₃), —CH₂C(CH₃)₂F, —C(═CH₂)(CF₃), —CH═C(CH₃)₂, —OCF₃, —C(CH₃)₂(CH₂OH), —C(CH₃)(CF₃)(CH₂OH), —C(CH₃)(CF₃)(C(O)OH), —C(O)OCH₃, —Si(CH₃)₃, phenyl, 1-methylcyclopropyl, 1-trifluoromethylcyclopropyl, cyclobutyl, 1-methylcyclobutyl, 3,3-difluoro-1-methylcyclobutyl, cyclopentyl, 1-methylcyclopentyl, 1-trifluoromethylcyclopentyl, 3,3-difluoro-1-methylcyclopentyl, 4,4-difluoro-1-methylcyclohexyl,

137. The compound of claim 123, or a pharmaceutically acceptable salt thereof, wherein R^{5b} is H, halo, C₁-C₆ alkyl, C₁-C₆ haloalkyl, C₁-C₆ alkoxy, or C₃-C₆ cycloalkyl;
optionally wherein R^{5b} is H, F, Cl, —CH₃, —CH₂CH₃, —CH(CH₃)₂, —C(CH₃)₃, —CF₃, —OCH₃, or cyclopropyl.

138. The compound of claim 123, wherein the compound is selected from:
or a pharmaceutically acceptable salt thereof.

139. The compound of claim 123, wherein the compound is selected from:
or a pharmaceutically acceptable salt thereof.

140. A pharmaceutical composition comprising the compound of claim 123, or a pharmaceutically acceptable salt thereof, and one or more pharmaceutically acceptable carriers or vehicles.

141. A method of inhibiting a voltage-gated sodium channel in a subject comprising administering to the subject the compound of claim 123, or a pharmaceutically acceptable salt thereof.

142. A method of treating or lessening the severity in a subject of pain comprising administering to the subject an effective amount of the compound of claim 123, or a pharmaceutically acceptable salt thereof.

143. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of claim 123, or a pharmaceutically acceptable salt thereof.

144. The compound of claim 123, wherein the compound is selected from:
or a pharmaceutically acceptable salt thereof.

145. A pharmaceutical composition comprising the compound of claim 144, or a pharmaceutically acceptable salt thereof, and one or more pharmaceutically acceptable carriers or vehicles.

146. A method of inhibiting a voltage-gated sodium channel in a subject comprising administering to the subject the compound of claim 144, or a pharmaceutically acceptable salt thereof.

147. A method of treating or lessening the severity in a subject of pain comprising administering to the subject an effective amount of the compound of claim 144, or a pharmaceutically acceptable salt thereof.

148. A method of treating or lessening the severity in a subject of chronic pain, gut pain, neuropathic pain, musculoskeletal pain, acute pain, inflammatory pain, cancer pain, idiopathic pain, postsurgical pain, visceral pain, multiple sclerosis, Charcot-Marie-Tooth syndrome, incontinence, pathological cough, or cardiac arrhythmia comprising administering to the subject an effective amount of the compound of claim 144, or a pharmaceutically acceptable salt thereof.

149. The compound of claim 123, wherein the compound is:
or a pharmaceutically acceptable salt thereof.

150. The compound of claim 123, wherein the compound is:
or a pharmaceutically acceptable salt thereof.

151. The compound of claim 123, wherein the compound is:
or a pharmaceutically acceptable salt thereof.

152. The compound of claim 123, wherein the compound is:
or a pharmaceutically acceptable salt thereof.

153. The compound of claim 123, wherein the compound is:
or a pharmaceutically acceptable salt thereof.

154. A compound of formula (I-D):
or a pharmaceutically acceptable salt thereof, wherein:
R^{6a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —NHC(O)NH₂, —NHC(O)NH(C₁-C₆ alkyl), —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), —C(NH)NH₂, —C(S)NH₂, —SO₂NH₂, or 5-10 membered heteroaryl comprising 1-3 heteroatoms selected from nitrogen, oxygen, and sulfur, wherein said heteroaryl is optionally substituted with 1-2 R^{a′};
R^{9a} is H, halo, —CN, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)₂, —OH, —(C₁-C₆ alkylene)-OH, —(C₁-C₆ alkylene)-(C₁-C₆ alkoxy), —(C₁-C₆ alkylene)-NH₂, —(C₁-C₆ alkylene)-NH(C₁-C₆ alkyl), —(C₁-C₆ alkylene)-N(C₁-C₆ alkyl)₂, —O(C₁-C₆ alkylene)-(C₁-C₆ alkoxy), —O(C₁-C₆ alkylene)-NH₂, —O(C₁-C₆ alkylene)-NH(C₁-C₆ alkyl), —O(C₁-C₆ alkylene)-N(C₁-C₆ alkyl)₂, —C(O)(C₁-C₆ alkyl), —C(O)NH₂, —C(O)NH(C₁-C₆ alkyl), —C(O)N(C₁-C₆ alkyl)₂, —C(O)OH, —C(O)O(C₁-C₆ alkyl), or —C(S)NH₂;
each R^{a′} is independently halo, —OH, C₁-C₆ alkyl, C₁-C₆ haloalkyl, —C(O)O(C₁-C₆ alkyl), or —C(O)NH₂;
X_{2b} is N or CR^{2b};
X_{5b} is N or CR^{5b};
X_{6b} is N or CR^{6b};
Z is a 5-7 membered aromatic or nonaromatic ring optionally containing 1-3 heteroatoms selected from nitrogen and oxygen and is optionally substituted with one or more R^{z};
R^{2b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —C(O)O(C₁-C₆ alkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{5b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —OH, —NH₂, —NH(C₁-C₆ alkyl), —N(C₁-C₆ alkyl)₂, —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo;
R^{6b} is H, halo, C₁-C₆ alkyl, C₁-C₆ alkenyl, C₁-C₆ haloalkyl, C₁-C₆ haloalkenyl, C₁-C₆ alkoxy, C₁-C₆ haloalkoxy, —NH₂, —NH(C₁-C₆ alkyl), —N(C₁-C₆ alkyl)(C₂-C₆ alkyl), —(C₁-C₆ alkylene)-OH, —(C₁-C₆ alkylene)-C(O)OH, —C(O)(C₁-C₆ alkyl), —C(O)(C₁-C₆ haloalkyl), —Si(C₁-C₆ alkyl)₃, C₃-C₆ cycloalkyl, (C₁-C₆ alkyl)-(C₃-C₆ cycloalkyl)-, or (C₁-C₆ haloalkyl)-(C₃-C₆ cycloalkyl)-, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halo; and
R^{z} is halo, C₁-C₆ alkyl, C₁-C₆ alkenyl, C₁-C₆ alkoxy, C₁-C₆ haloalkenyl, —CN, —C(O)NH₂, —C(O)NH(C₁-C₆ alkyl), —C(O)N(C₁-C₆ alkyl)₂, or —C(O)OH.

155. A compound of formula (II):
or a pharmaceutically acceptable salt thereof, wherein:
R^{3c} is H, halo, or C₁-C₆ alkyl;
R^{4c} is H, halo, —CN, or C₁-C₆ alkoxy;
R^{5c} is H, halo, —CN, C₁-C₆ alkyl, C₁-C₆ haloalkyl, C₁-C₆ alkoxy, or —C(O)O(C₁-C₆ alkyl);
R^{6c} is H, halo, —OH, —CN, C₁-C₆ alkoxy, C(O)NH₂, or 5-10 membered heteroaryl comprising 1-3 heteroatoms selected from nitrogen and oxygen;
R^{9c} is H, C₁-C₆ alkyl, —(C₁-C₆ alkylene)-OH, —(C₁-C₆ alkylene)-O(C₁-C₆ alkyl), or —C(O)O(C₁-C₆ alkyl);
Y is
X_{3d} is N or CR^{3d};
R^{2d}, R^{3d}, and R^{4d} are defined as follows:
(i) R^{2d} is H, halo, —OH, C₁-C₆ alkyl, C₁-C₆ alkoxy, C₁-C₆ haloalkoxy, or —(C₁-C₆ alkylene)-OH;
R^{3d} is H, halo, C₁-C₆ alkyl, C₁-C₆ haloalkyl, C₁-C₆ alkoxy, or C₁-C₆ haloalkoxy; and
R^{4d} is H, halo, C₁-C₆ alkyl, C₁-C₆ haloalkyl, C₁-C₆ haloalkenyl, C₁-C₆ alkoxy, C₁-C₆ haloalkoxy, —(C₁-C₆ alkylene)-OH, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN; or
(ii) R^{2d} and R^{3d}, together with the carbon atoms to which they are attached, form a ring of formula:
and
R^{4d} is H, halo, C₁-C₆ alkyl, C₁-C₆ haloalkyl, C₁-C₆ haloalkenyl, C₁-C₆ alkoxy, C₁-C₆ haloalkoxy, —(C₁-C₆ alkylene)-OH, —(C₁-C₆ alkylene)-(C₁-C₆ alkoxy), —Si(C₁-C₆ alkyl)₃, —C(O)O(C₁-C₆ alkyl), (C₁-C₆ alkyl)-(C₃-C₆ cycloalkyl)-, (C₁-C₆ haloalkyl)-(C₃-C₆ cycloalkyl)-, or C₃-C₆ cycloalkyl, wherein cycloalkyl in said C₃-C₆ cycloalkyl, (C₁-C₆ alkyl)-(C₃-C₆ cycloalkyl)-, or (C₁-C₆ haloalkyl)-(C₃-C₆ cycloalkyl)- is optionally substituted with one or more halogen or —CN; or
(iii) R^{2d} is H, halo, —OH, C₁-C₆ alkyl, C₁-C₆ alkoxy, C₁-C₆ haloalkoxy, or —(C₁-C₆ alkylene)-OH; and
R^{3d} and R^{4d}, together with the carbon atoms to which they are attached, form a ring of formula:
R^{5d} is H, halo, C₁-C₆ alkyl, C₁-C₆ haloalkyl, C₁-C₆ alkoxy, or —C(O)(C₁-C₆ alkyl);
R^{6d} is H, halo, or C₁-C₆ alkyl;
R^{7d} is C₁-C₆ alkyl; and
R^{9d} is C₁-C₆ alkyl;
provided that:
(i) if Y′ is
and X_{3d} is CR^{3d}, then no more than four of R^{2d}, R^{3d}, R^{4d}, R^{5d}, and R^{6d} are H; and
(ii) if Y′ is
and X_{3d} is N, then no more than three of R^{2d}, R^{4d}, R^{5d}, and R^{6d} are H.

156. A compound of formula (III):
or a pharmaceutically acceptable salt thereof, wherein:
X₃ₖ is N or CH;
X₄ₖ is N or CH;
X₅ₖ is N or CR^{5k};
X₆ₖ is N, N⁺—O⁻, or CR^{6k};
R^{5k} is H, C₁-C₆ alkoxy, —OH, —OCH₂CH₂N(CH₃)₂, or —N(CH₃)(CH₂CH₂OCH₃);
R^{6k} is H, —OH, C₁-C₆ alkoxy, or —C(O)NH₂;
R^{2L} is C₁-C₆ alkyl;
R^{4L} is C₁-C₆ alkyl, C₁-C₆ haloalkyl, or (C₁-C₆ haloalkyl)-(C₃-C₆ cycloalkyl)-; and
R^{5L} is H, halo, or C₁-C₆ alkyl,
provided that:
(i) at least one of X₃ₖ, X₄ₖ, and X₅ₖ is N, or X₆ₖ is N or N⁺—O⁻; and
(ii) no more than two of X₃ₖ, X₄ₖ, X₅ₖ, and X₆ₖ are N; and
(iii) if X₆ₖ is N⁺—O⁻, then X₃ₖ and X₄ₖ are CH, and X₅ₖ is CR^{5k}; and
(iv) if X₅ₖ is N, then X₄ₖ is N.
